(19) United States
(12) Patent Application Publication
(10) Pub. No.: US 2026/0176576 A1
(43) Pub. Date: Jun. 25, 2026

(54) AGONIST EXTRACELLULAR VESICLES

(71) Applicant: Diadem Biotherapeutics, Inc., Torrance, CA (US)

(72) Inventors: Mickey PENTECOST, West Hollywood, CA (US); Wojciech BARTKOWSKI, La Crescenta, CA (US); Rakesh VERMA, Boston, MA (US); Dulce ALVARADO, Lynwood, CA (US)

(21) Appl. No.: 19/259,672

(22) Filed: Jul. 3, 2025

Related U.S. Application Data
(63) Continuation of application No. PCT/US24/10433, filed on Jan. 5, 2024.
(60) Provisional application No. 63/437,461, filed on Jan. 6, 2023.
(60) Provisional application No. 63/546,967, filed on Nov. 2, 2023.

(51) Int. Cl. C12N 5/00; C12N 15/85

(52) U.S. Cl. CPC C12N 5/0006; C12N 15/85; C12N 2510/02; C12N 2800/107

(57) ABSTRACT

Described herein are compositions and techniques related to generation and therapeutic application of engineered extracellular vesicles. Engineered extracellular vesicles are vesicles (i.e., exosomes) comprising at least one engineered fusion polypeptide further comprising a vesicle targeting domain, scaffold linker domain, and signaling domain (i.e., a multi-effector signaling domain) wherein the signaling domain targets and interacts with a biological target, such as cell surface receptors. Said engineered fusion proteins can be organized in genetic vector constructs, expressed in mammalian cells, wherein the vesicle targeting domains anchor to extracellular vesicles such as exosomes, thereby presenting their joined multi-effector signaling domain which can relay a signal to target cells. Engineered extracellular vesicles adopt the hallmark biophysical and biochemical features of extracellular vesicles, allowing for rapid deployment and scale-up. Importantly, this strategy can allow for kinetically favorable signal generation and signal propagation to a target cell.

### CROSS-REFERENCE TO RELATED APPLICATIONS

This application is a continuation of International Application No. PCT/US2024/010433, filed Jan. 5, 2024, which claims the benefit of U.S. Provisional Patent Application No. 63/437,461 filed Jan. 6, 2023, and U.S. Provisional Patent Application No. 63/546,967, filed Nov. 2, 2023, each of which is hereby incorporated by reference in its entirety.

### REFERENCE TO SEQUENCE LISTING

This application contains a Sequence Listing submitted electronically in XML format and is hereby incorporated by reference in its entirety. Said XML copy, created on Jul. 3, 2025, is named “69749-702-301_SL_ST26.xml” and is 481,168 bytes in size.

### FIELD OF THE INVENTION

This invention relates to the generation of extracellular vesicles, including features of extracellular vesicles engineered to deliver signaling, for therapeutic use, including treatment of immune diseases and cancer.

### BACKGROUND

Extracellular vesicles (EVs) are membrane bound lipid particles that are released from various cell types that function to transfer “cargo” such as nucleic acids and proteins to other cells. EVs are vesicles that play a critical role in intercellular communication by transferring various microRNAs, growth factors, cytokines, lipids, and proteins to cells impacting cellular processes in the recipient or target cell. EVs are not able to replicate but serve as cell messengers. EV-mediated signals can be transmitted by all the different biomolecule categories—protein, lipids, nucleic acids, and sugars—and the unique package of this information provides both protection and the option of simultaneous delivery of multiple different messengers even to sites remote to the vesicular origin. See, e.g., Yáñez-Mó M, Siljander P R, Andreu Z, et al. Biological properties of extracellular vesicles and their physiological functions. J Extracell Vesicles. 2015; 4:27066. Published 2015 May 14. Doi: 10.3402/jev.v4.27066, which is incorporated herein by reference in its entirety.

EV surface displayed proteins (e.g., integrins and tetraspanins) and cell surface displayed proteins (e.g., target receptors) also facilitate interaction with target cells and determine binding specificity and mediate intercellular communication. Such communication between cells and EVs lead to various physiological and pathological process including but not limited to tissue repair, tumor progression, and immune response modulation. There is an increasing amount of evidence that shows that EVs can modulate a milieu of cellular signaling processes. See, e.g., Yadid et al. Science Translation Medicine (2020); Cerqueira de Abreu et al. Nature Reviews Cardiology (2020); Zhang W. et al. Protein J. (2019); Zha Q B et al. Tumor Biology. February 2017; Tan et al. (2016) Recent advances of exosomes in immune modulation and autoimmune diseases, Autoimmunity, 49:6, 357-365; Kalluri R, LeBleu V S. et al. The biology, function, and biomedical applications of exosomes. Science. 2020 Feb. 7; 367 (6478). which are incorporated herein by reference in their entirety.

Because of their ability of targeted and efficient delivery of intercellular communication EVs have gained significant attention as a potential therapeutic modality. For example, the delivery of fusion polypeptides displayed on an EV surface is a highly promising strategy as a therapeutic platform in many contexts, exploiting the unique biophysical and biochemical characteristics of EVs. However, there remains a great need in the art for a flexible and dynamic platform of engineered EV fusion polypeptide display, where specific biological signals can be reliably targeted to a target cell without off-target effects and that provide a robust cellular response to achieve a therapeutic effect, such as modulating the immune system.

### SUMMARY OF THE INVENTION

In various embodiments, the present invention provides an engineered extracellular vesicle comprising at least one fusion polypeptide. The fusion polypeptide comprises: an agonistic multi-effector domain in an exterior position relative to a phospholipid bilayer of the extracellular vesicle; a polypeptide linker; and at least one vesicle targeting domain spanning at least partly through the phospholipid bilayer of the extracellular vesicle. The polypeptide linker is positioned between the multi-effector domain and the at least one vesicle targeting domain. The agonistic multi-effector domain comprises at least one fragment from one or more protein of interest.

In some embodiments, the at least one vesicle targeting domain is a Type II transmembrane protein or fragment thereof, or a multi-pass transmembrane protein or fragment thereof.

In some embodiments, the Type II transmembrane protein or fragment thereof comprises 4F2 (CD98 heavy chain) or CD298.

In some embodiments, the multi-pass transmembrane protein is a tetraspanin or fragment thereof.

In some embodiments, the at least one vesicle targeting domain further comprises at least one lipid anchoring domain.

In some embodiments, the at least one lipid anchoring domain further comprises a myristoylation and/or palmitoylation sequence.

In some embodiments, the polypeptide linker is positioned C-terminus relative to the at least one vesicle targeting domain.

In some embodiments, the polypeptide linker is positioned N-terminus relative to the agonistic multi-effector domain.

In some embodiments, the at least one vesicle targeting domain is a Type I transmembrane protein or fragment thereof, or a multi-pass transmembrane protein or fragment thereof.

In some embodiments, the multi-pass transmembrane protein is a tetraspanin or fragment thereof.

In some embodiments, the tetraspanin is CD9 or fragment thereof.

In some embodiments, the fragment of CD9 is CD9tm2.

In some embodiments, the polypeptide linker is positioned N-terminus relative to the at least one vesicle targeting polypeptide.

In some embodiments, the polypeptide linker is positioned C-terminus relative to the agonistic multi-effector domain.

In some embodiments, the at least one vesicle targeting domain comprises a secretion signal recognition sequence.

In some embodiments, the secretion signal recognition sequence is positioned N-terminus relative to the agonistic multi-effector domain.

In some embodiments, the secretion signal recognition sequence is proteolytically cleaved from the agonistic multi-effector domain.

In some embodiments, a CVIM motif from KRAS-13 (CAAX box) is at the C-terminus of the fusion polypeptide.

In some embodiments, the CVIM motif is farnesylated by a post translational modification, an isoprenyl group is added to the cysteine residue, and the VIM is cleaved via proteolysis.

In some embodiments, the engineered extracellular vesicle further comprises a linker between the agonistic multi-effector domain and the polypeptide linker.

In some embodiments, the engineered extracellular vesicle further comprises a linker between the polypeptide linker and the at least one vesicle targeting domain.

In some embodiments, the engineered extracellular vesicle further comprises a monomer linker between each monomer of the agonistic multi-effector domain.

In some embodiments, the agonistic multi-effector domain comprises at least three fragments from one or more protein of interest.

In some embodiments, each fragment of the agonistic multi-effector domain is a tumor necrosis factor (TNF) homology domain (THDs) or a fragment thereof.

In some embodiments, the THD is derived from a TNF superfamily member (TNFSF) selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, TWEAK or a fragment thereof.

In some embodiments, the linker between the agonistic multi-effector domain and the polypeptide linker, the linker between the polypeptide linker and the at least one vesicle targeting domain, or the linker between each fragment is each independently selected from the group consisting of ID, GSSG (SEQ ID NO: 154), G, GS, GGS, GGGS (SEQ ID NO: 218), GGGGS (SEQ ID NO:156), (GGGGS) n wherein n is an integer between 1 and 10, and combinations thereof.

In some embodiments, the polypeptide linker comprises Fc or Fc mutein.

In some embodiments, the extracellular vesicle is an exosome.

In various embodiments, the present invention provides a composition comprising a plurality of the engineered extracellular vesicles described above. In some embodiments, the composition further comprises a pharmaceutically acceptable carrier.

In various embodiments, the present invention provides an engineered extracellular vesicle comprising at least one fusion polypeptide. The fusion polypeptide comprises a signaling domain in an exterior position relative to a phospholipid bilayer of the extracellular vesicle; a polypeptide linker; and at least one vesicle targeting domain spanning at least partly through the phospholipid bilayer of the extracellular vesicle. The polypeptide linker is positioned between the multi-effector domain and the at least one vesicle targeting domain. The signaling domain comprises a fragment from a protein of interest.

### BRIEF DESCRIPTION OF THE DRAWINGS

FIG. 1 is a schematic diagram of a type II membrane protein fusion polypeptide comprising a vesicle targeting domain, a multi-effector domain, and a plurality of linkers. The N-Terminus being on the interior of the extracellular vesicle while the C-Terminus being on the exterior.

FIG. 2 is a schematic diagram of an engineered extracellular vesicle displaying one or more type II membrane protein fusion polypeptides according to various embodiments of the present invention.

FIG. 3A is a schematic diagram of a type II membrane protein fusion polypeptide comprising a 4F2 vesicle targeting domain, a multi-effector domain, and a plurality of linkers. The N-Terminus being on the interior of the extracellular vesicle while the C-Terminus being on the exterior.

FIG. 3B shows a Type II membrane protein fusion polypeptide displayed on the surface of an engineered extracellular vesicle comprising a multi-effector domain, a 4F2 vesicle targeting domain, and a plurality of linkers, the C-Terminus being on the exterior of the extracellular vesicle while the N-Terminus being on the interior.

FIG. 4A is a schematic diagram of a type II membrane protein fusion polypeptide comprising a multi-effector domain, a CD298 vesicle targeting domain, and a plurality of linkers. The N-Terminus being on the interior of the extracellular vesicle while the C-Terminus being on the exterior of the extracellular vesicle.

FIG. 4B shows a Type II membrane protein fusion polypeptide displayed on the surface of an engineered extracellular vesicle comprising a CD298 vesicle targeting domain, a multi-effector domain, and a plurality of linkers, the C-Terminus being on the exterior of the extracellular vesicle while the N-Terminus being on the interior of the extracellular vesicle.

FIG. 5A is a schematic diagram of a type II membrane protein fusion polypeptide comprising a multi-effector domain, a 4F2 vesicle targeting domain, an Fc linker, and a plurality of linkers. The N-Terminus being on the interior of the extracellular vesicle while the C-Terminus being on the exterior of the extracellular vesicle.

FIG. 5B shows a Type II membrane protein fusion polypeptide displayed on the surface of an engineered extracellular vesicle comprising a multi-effector domain, a 4F2 vesicle targeting domain, an Fc linker, and a plurality of linkers, the C-Terminus being on the exterior of the of the extracellular vesicle while the N-Terminus being on the interior of the extracellular vesicle.

FIG. 6A is a schematic diagram of a type II membrane protein fusion polypeptide comprising a multi-effector domain, a CD298 vesicle targeting domain, an Fc linker, and a plurality of linkers. The N-Terminus being on the interior of the extracellular vesicle while the C-Terminus being on the exterior of the extracellular vesicle.

FIG. 6B shows a Type II membrane protein fusion polypeptide displayed on the surface of an engineered extracellular vesicle comprising a multi-effector domain, a CD298 vesicle targeting domain, an Fc linker, and a plurality of linkers, the C-Terminus being on the exterior of the extracellular vesicle while the N-Terminus being on the interior of the extracellular vesicle.

FIG. 7 shows a diagram of domain organization of fusion polypeptide embodiments.

FIG. 8A shows Dot Blot analysis of size exclusion chromatography (SEC) fractions after purification of extracellular vesicles engineered to display various fusion polypeptide embodiments.

FIG. 8B shows the background subtracted integrated intensity (I.I.) of SEC fraction 8 spots.

FIG. 9A shows Dot Blot analysis of size exclusion chromatography (SEC) fractions after purification of extracellular vesicles engineered to display various fusion polypeptide embodiments.

FIG. 9B A cartoon representation of the fusion polypeptide M/P-CD298-sc4-1BBL.

FIG. 10 shows size versus concentration histograms of purified extracellular vesicles from unmodified cells or purified extracellular vesicles from cells engineered to display engineered fusion polypeptides.

FIG. 11 shows analysis of extracellular vesicle epitopes on unmodified extracellular vesicles and extracellular vesicles displaying fusion polypeptide embodiments using antibody-conjugated capture beads and flow cytometry.

FIG. 12 shows quantification of unmodified extracellular vesicles and extracellular vesicles engineered to display a preferred fusion polypeptide embodiment bound to tetraspanin antibody-conjugated interferometric reflectance imaging sensors and counterstained for combined immunofluorescence imaging and single particle interferometry.

FIG. 13 shows images of unmodified extracellular vesicles and extracellular vesicles engineered to display a preferred fusion polypeptide embodiment bound to CD81 antibody-conjugated interferometric reflectance imaging sensors and counterstained for 4-1BBL.

FIG. 14A shows Dot Blot analysis of size exclusion chromatography (SEC) fractions after purification of unmodified extracellular vesicles and extracellular vesicles engineered to display a preferred fusion polypeptide embodiment.

FIG. 14B shows total protein plots of SEC fractions from purification of unmodified extracellular vesicles.

FIG. 14C shows total protein plots of SEC fractions from purification of extracellular vesicles engineered to display a preferred fusion polypeptide embodiment.

FIG. 15 shows analysis of extracellular vesicle epitopes on unmodified extracellular vesicles and extracellular vesicles engineered to display fusion polypeptide embodiments using antibody-conjugated capture beads and flow cytometry.

FIG. 16 shows size versus concentration histograms of extracellular vesicles purified from unmodified cells and engineered extracellular vesicles purified from cells engineered to display a preferred embodiment of a fusion polypeptide.

FIG. 17 shows quantification of unmodified extracellular vesicles and extracellular vesicles engineered to display a preferred fusion polypeptide embodiment bound to tetraspanin antibody-conjugated interferometric reflectance imaging sensors and counterstained for combined immunofluorescence imaging and single particle interferometry.

FIG. 18 shows images of unmodified extracellular vesicles and extracellular vesicles engineered to display a preferred fusion polypeptide embodiment bound to CD81 antibody-conjugated interferometric reflectance imaging sensors and counterstained for 4-1BBL.

FIG. 19A shows Dot Blot analysis of size exclusion chromatography (SEC) fractions after purification of extracellular vesicles engineered to display a preferred fusion polypeptide embodiment.

FIG. 19B shows analysis of extracellular vesicle epitopes on unmodified extracellular vesicles and extracellular vesicles displaying fusion polypeptide embodiments using antibody-conjugated capture beads and flow cytometry.

FIG. 20 shows an assay system comprising of a genetically engineered Jurkat T cell line that expresses human TNF receptor superfamily member (TNFRSF) and a luciferase reporter driven by a response element that can respond to ligand or agonist stimulation of the TNFRSF receptor.

FIG. 21A shows relative 4-1BB signaling (i.e., agonism) as indicated in relative light units (RLU) of effector cells induced by cells displaying various fusion polypeptide embodiments and by cells engineered to display native human 4-1BBL and by unmodified cells (i.e., HEK293 cells).

FIG. 21B shows relative 4-1BB signaling (i.e., agonism) as indicated in relative light units (RLU) of effector cells induced by filtered media collected after the growth of cells displaying various embodiments of fusion polypeptides and by filtered media collected after the growth of cells engineered to display native human 4-1BBL and by filtered media collected after the growth of unmodified cells (i.e., HEK293 cells).

FIG. 21C shows relative 4-1BB signaling (i.e., agonism) as indicated in relative light units (RLU) of effector cells induced by SEC purified engineered extracellular vesicles displaying various embodiments of fusion polypeptide and by SEC purified engineered extracellular vesicles displaying native 4-1BBL and by SEC purified unmodified extracellular vesicles.

FIG. 22A shows the concentration of 4-1BBL per μg of purified extracellular vesicle from HEK293 cells engineered to display various fusion polypeptide embodiments, extracellular vesicle from HEK293 cells engineered to display native 4-1BBL, and extracellular vesicle from unmodified HEK293 cells.

FIG. 22B shows schematic diagrams of the fusion polypeptide embodiments presented in FIG. 22A.

FIG. 23 shows relative 4-1BB signaling (i.e., agonism) as indicated in relative light units (RLU) of effector cells induced by SEC purified extracellular vesicles displaying various embodiments of fusion polypeptide and control unmodified extracellular vesicles.

FIG. 24A shows 4-1BB signaling (i.e., agonism) induced by the extracellular vesicles displaying the fusion polypeptide embodiment or unmodified extracellular vesicles presented herein at the indicated concentrations in a 4-1BB signaling bioassay system.

FIG. 24B shows schematic diagrams of the fusion polypeptide embodiments presented in FIG. 24A.

FIG. 25 shows a comparison of 4-1BB signaling (i.e., agonism) induced by extracellular vesicles expressing the fusion polypeptide embodiment M/P-4F2-Fc-sc4-1BBL, a recombinant Fc tagged ligand trimer, and Urelumab antibody biosimilar.

FIG. 26 shows GITR signaling (i.e., agonism) induced by extracellular vesicles displaying the fusion polypeptide embodiment M/P-4F2-Fc-scGITR compared to GITR signaling (i.e., agonism) induced by a Ragifilimab antibody biosimilar.

FIG. 27 shows OX40 signaling (i.e., agonism) induced by extracellular vesicles displaying the fusion polypeptide embodiment M/P-4F2-Fc-scOX40L, compared to OX40 signaling (i.e., agonism) induced by a Ivuxolimab antibody biosimilar.

FIG. 28 shows the relative 4-1BB signaling (i.e., agonism) induced by vehicle (DPBS) and engineered extracellular vesicles displaying the fusion polypeptide embodiment presented herein.

FIG. 29 shows the relative GITR signaling (i.e., agonism) induced by vehicle (DPBS) and engineered extracellular vesicles displaying the fusion polypeptide embodiment presented herein or native unmodified extracellular vesicles.

FIG. 30 shows the relative OX40 signaling (i.e., agonism) induced by vehicle (DPBS) and engineered extracellular vesicles displaying the fusion polypeptide embodiment presented herein.

FIG. 31 shows modulation of antigen-specific memory T cell expansion and function.

FIG. 32 shows flow cytometry plots used to assess modulation of antigen-specific memory T cell expansion and function.

FIG. 33 shows real time cell analysis (RTCA) using impedance biosensors in specialized microplate wells (E-Plates, Agilent) to monitor the status of adherent cancer target cells (A, B, C, D) in the presence of non-adherent T cells.

FIG. 34 shows individual and mean tumor volumes for intratumoral treatment of subcutaneous MC38 Colon Carcinoma Model in B-h4-1BB mice.

FIG. 35 shows Kaplan-Meier survival curves for intratumoral treatment of subcutaneous MC38 Colon Carcinoma Model in B-h4-1BB mice.

FIG. 36 shows measurement of liver enzymes for intratumoral treatment of subcutaneous MC38 Colon Carcinoma Model in B-h4-1BB mice.

FIG. 37 shows hematoxylin and eosin staining of tumor margin sections of subcutaneous MC38 Colon Carcinoma Model in B-h4-1BB mice.

FIG. 38 shows a general method of purifying extracellular vesicles.

FIG. 39 illustrates TNF superfamily ligands (TNFSF), TNF receptor superfamily (TNFRSF), and their primary cellular targets.

### DETAILED DESCRIPTION

The compositions and methods provided herein are based, in part, on the discovery that engineered extracellular vesicles (e.g., exosomes) displaying an engineered fusion protein (e.g., M/P-4F2-Fc-4-1BBL) reduces tumor volume growth rate and increases duration of survival of mice in an MC38 colon carcinoma model showing that engineered fusion polypeptides presented herein are an effective therapeutic for the treatment of cancer. The compositions and methods provided herein are further based, in part, on the discovery that engineered fusion polypeptides displayed on engineered extracellular vesicles produce enhanced signaling compared to an equal quantity of recombinant ligand or agonist antibody targeting the same receptor. Since some cellular receptors, (e.g., 4-1BB) require higher order clustering or super-clustering to promote a signaling response, it stands to reason that extracellular vesicles engineered to display a plurality of ligands on their surface wherein the ligands may engage target receptors on target cells and promote clustering of said target receptors thereby promoting a signal response on said target cell.

In various aspects provided herein are engineered extracellular vesicles comprising at least one fusion polypeptide, the at least one fusion polypeptide comprising an agonistic multi-effector domain in an exterior position relative to a phospholipid bilayer of the extracellular vesicle; a polypeptide linker; and at least one vesicle targeting domain spanning at least partly through the phospholipid bilayer of the extracellular vesicle. The polypeptide linker is positioned between the agonistic multi-effector domain and the at least one vesicle targeting polypeptide. In some embodiments of any of the aspects, the polypeptide linker comprises Fc or Fc mutein. In some embodiments of any of the aspects, the at least one vesicle targeting domain comprises a Type II transmembrane protein or fragment thereof, or a multi-pass transmembrane protein or fragment thereof. In some embodiments of any of the aspects, the at least one vesicle targeting domain is the membrane anchoring domain of a Type II transmembrane protein or fragment thereof, or the membrane anchoring domain of a multi-pass transmembrane protein or fragment thereof. In various embodiments, a fragment of a

membrane anchoring domain or a fragment of a multi-pass transmembrane domain can be at least 75%, 80%, 85%, 90%, or 95% of a membrane anchoring domain or at least 75%, 80%, 85%, 90%, or 95% of a multi-pass transmembrane domain. In some embodiments of any of the aspects, the multi-pass transmembrane protein is a tetraspanin or fragment thereof. In some embodiments of any of the aspects, the vesicle targeting domain further comprises a myristoylation and/or palmitoylation motif or moiety. In some embodiments of any of the aspects, the polypeptide linker is positioned C-terminus relative to the at least one vesicle targeting domain. In some embodiments of any of the aspects, the polypeptide linker is positioned N-terminus relative to the agonistic multi-effector domain. In some embodiments of any of the aspects, the fusion polypeptide comprises an agonistic multi-effector domain, wherein the agonistic multi-effector domain further comprises a single chain (sc) polypeptide of tumor necrosis factor (TNF) superfamily (TNFSF) trimers, and the fusion polypeptide is displayed on the engineered extracellular vesicle. For example, the TNFSF members include GITRL, 4-1BBL, and OX40L.

TNFRSF agonist antibodies that bind to Fcγ receptors are more likely to cause toxicities than agonists that lack Fcγ receptor binding. Agonist antibodies have a bell-shaped dose response curve, which limits maximal receptor activation and complicates optimal dose range-finding. Agents containing pre-formed TNF ligand trimers demonstrate more potent agonist activity than antibodies. Engineered extracellular vesicles displaying fusion polypeptides demonstrate more effective dose-dependent pharmacodynamics per active molecule by providing higher-order TNFRSF clustering. Extracellular vesicles displaying fusion polypeptides can be engineered for binding or targeting to specific cell types depending on the fusion polypeptide protein of interest. Engineered extracellular vesicles displaying fusion polypeptides can facilitate higher-order receptor clustering, expanding the therapeutic window for agonist immunotherapy when compared to antibodies.

In some embodiments, the at least one vesicle targeting domain of the fusion polypeptide is a membrane anchoring domain from a Type I transmembrane protein or fragment thereof, or a multi-pass transmembrane protein or fragment thereof. In some embodiments of any of the aspects, the multi-pass transmembrane protein is a tetraspanin or fragment thereof. In various embodiments, a fragment of a membrane anchoring domain or a fragment of a multi-pass transmembrane domain can be at least 75%, 80%, 85%, 90%, or 95% of a membrane anchoring domain or at least 75%, 80%, 85%, 90%, or 95% of a multi-pass transmembrane domain. In some embodiments of any of the aspects, the polypeptide linker is positioned N-terminus relative to the at least one vesicle targeting domain. In some embodiments of any of the aspects, the polypeptide linker is positioned C-terminus relative to the agonistic multi-effector domain. In some embodiments of any of the aspects, the vesicle targeting polypeptide comprises a secretion signal recognition sequence. In some embodiments of any of the aspects, the secretion signal recognition sequence is positioned N-terminus relative to the agonistic multi-effector domain. In some embodiments of any of the aspects, the secretion signal recognition sequence is proteolytically cleaved from the engineered fusion polypeptide. The secretion signal recognition sequence is cleaved off by cellular proteases after translation, and thus, the final fusion polypeptide on the surface of the extracellular vesicle does not have the signal recognition sequence because the secretion signal recognition sequence is there only for membrane localization during the translation.

In some embodiments of any of the aspects, the engineered extracellular vesicle further comprises a linker between the agonistic multi-effector domain and the polypeptide linker. In some embodiments of any of the aspects, the engineered extracellular vesicle further comprises a linker between the polypeptide linker and the at least one vesicle targeting domain. In some embodiments of any of the aspects, the engineered extracellular vesicle further comprises a monomer linker between each monomer of the agonistic multi-effector domain. In some embodiments of any of the aspects, the multi-effector domain comprises three agonist signaling domains. In some embodiments of any of the aspects, each agonist signaling domain of the multi-effector domain is a tumor necrosis factor (TNF) homology domain (THDs) or a fragment thereof. In some embodiments of any of the aspects, the THD is from a TNFSF member selected from the group consisting of TNFα, TNFβ, TNFγ, ED1-A1, EDI-A2, GITRL, 4-1BBL, OX40L, GITRL, LIGHT, CD27L, CD30L, CD40L, TRAIL, FASL, BAFF, APRIL, RANKL, TL1A, and TWEAK. In some embodiments of any of the aspects, the linker between the agonistic multi-effector domain and the polypeptide linker, the linker between the polypeptide linker and the at least one targeting polypeptide, or the monomer linker is each independently selected from the group consisting of ID, GSSG (SEQ ID NO: 154), G, GS, GGS, GGGS (SEQ ID NO: 218), GGGGS (SEQ ID NO:156), (GGGGS)n, wherein n is an integer between 1 and 10, and combinations thereof. In some embodiments of any of the aspects, the extracellular vesicle is an exosome.

In some embodiments of any of the aspects, a composition comprises a plurality of the engineered extracellular vesicles. In some embodiments of any of the aspects, the composition further comprises a pharmaceutically acceptable carrier. In some embodiments of any of the aspects, target receptor signaling is induced through binding and clustering. In some embodiments of any of the aspects, binding of the multi-effector domain of the fusion polypeptide to a target receptor (e.g., target protein) clusters the target receptor and induces signaling of the clustered target receptor. In some embodiments of any of the aspects, the target receptor is agonized through binding and clustering by the fusion polypeptide. In some embodiments of any of the aspects, binding of multi-effector domains of multiple fusion polypeptides displayed on an extracellular vesicle induces agonist signaling of the clustered target receptors. In some embodiments of any of the aspects, binding of multi-effector domains of multiple fusion polypeptides displayed on an extracellular vesicle to multiple target receptors induces clustering of said multiple target receptors and induces agonist signaling of the clustered target receptors.

Generally, the extracellular vesicles (e.g., exosomes) provided herein are produced by contacting a population of cells with a nucleic acid construct encoding the fusion polypeptides provided herein and isolating a plurality of extracellular vesicles. The extracellular vesicles can then be purified by methods provided herein and are formulated for therapeutic use, including but not limited to, for the treatment of autoimmune diseases, cancer, or modulating inflammation in a subject.

The compositions and methods provided herein are specifically designed to exploit the membrane trafficking mechanisms of extracellular vesicles and rely on the hallmark biophysical and biochemical properties of extracellular vesicles, such as exosomes. The extracellular vesicles provided herein are specifically engineered to induce/agonize and propagate biological signaling via a target protein (e.g., by activating a receptor or enzyme or agonizing said receptor or enzyme). Alternatively, the engineered extracellular vesicles provided herein can act as cellular decoys or to reduce or antagonize biological signaling, e.g., by blocking an endogenous ligand from binding to a target receptor on a cell and preventing activation of the receptor or blocking an endogenous receptor from binding to a target ligand on a cell and preventing activation of the receptor.

Engineering of the extracellular vesicles provided herein extends these capabilities significantly by incorporating vesicle targeting domains attaching to extracellular vesicles such as exosomes, further coupled with signaling effector domains of choice. For example, attachment of vesicle targeting domains to exosomes, along with their linked signaling effector domains, allows for receptor clustering for biological signal induction/agonism and propagation not otherwise possible. In this aspect, the aforementioned design achieves the aim of an engineered extracellular vesicle by inducing the desired biological signaling in a target recipient cell.

Various aspects and embodiments of the compositions and methods are provided herein in detail below.

The compositions provided herein comprises at least one extracellular vesicle (EV), wherein the extracellular vesicle comprises at least one fusion polypeptide or a plurality of fusion polypeptides.

Various embodiments of the present invention provide for an engineered extracellular vesicle comprising at least one fusion polypeptide, the fusion polypeptide comprising: an agonistic effector domain (e.g., multi-effector domain) in an exterior position relative to a phospholipid bilayer of the extracellular vesicle; a polypeptide linker; and at least one vesicle targeting polypeptide spanning at least partly through the phospholipid bilayer of the extracellular vesicle, wherein the polypeptide linker is positioned between the agonistic effector domain (e.g., multi-effector domain) and the at least one vesicle targeting domain.

### Vesicle Targeting Polypeptide

In various embodiments, the at least one vesicle targeting domain of the fusion polypeptide is a membrane anchoring domain from a Type I membrane protein or a fragment thereof. In some embodiments, the at least one vesicle targeting domain is a membrane anchoring domain from a Type II membrane protein or fragment thereof. In various embodiments, the at least one vesicle targeting domain is a membrane anchoring domain from a Type III membrane protein or a fragment thereof. In various embodiments, the at least one vesicle targeting domain is a membrane domain from a multi-pass transmembrane protein or fragment thereof. In various embodiments, the at least one vesicle targeting domain is a membrane anchoring domain from a multi-pass transmembrane protein or fragment thereof. In various embodiments, a fragment of a membrane anchoring domain or a fragment of a multi-pass transmembrane domain can be at least 75%, 80%, 85%, 90%, or 95% of a membrane anchoring domain or at least 75%, 80%, 85%, 90%, or 95% of a multi-pass transmembrane domain.

### Type I Membrane Protein Vesicle Targeting Domains

In some embodiments of any of the aspects, the vesicle targeting domain comprises amino acid sequences from a type I membrane protein. Exemplary type I membrane proteins include but are not limited to CD1a, CD1b, CD1c, CD1d, CD1e, LEU1 (CD5), CD6, CD7, CD10, ITGB2 (CD18), CD19, CR2 (CD21), CD27, CD28, CD34, integrin alpha-IIb (ITA2B, CD41), platelet glycoprotein IX (CD42a), platelet glycoprotein Ib alpha chain (CD42b), platelet glycoprotein Ib beta chain (CD42c), platelet glycoprotein V (CD42d), B7-1 (CD80), B7-2 (CD86), OX40 (CD134), glucocorticoid-induced TNFR-related protein (GITR, CD357), inducible T-cell costimulatory (ICOS, CD278), ICOS ligand (ICOSL, CD275), Herpes virus entry mediator A (HVEM, CD270), B7-H3 (CD276), B and T lymphocyte attenuator (BTLA, D272), CTLA-4 (CD152), killer cell immunoglobulin-like receptor family (KIR family, CD158 family: CD158a-k; KIR2DL1, KIR2DL2, KIR2DL3, KIR3DP1, KIR2DL4, KIR3DL1, KIRDs1, KIR2DL5A, KIR2D15B, KIR2DS5, KIR2DS1, KIR2DS4, KIR2DS2, KIR3DL2), PD-1 (CD279), PD-L1 (CD274), PD-L2 (CD273), T-cell immunoglobulin mucin receptor 1 (TIM-1, CD365), T-cell immunoglobulin mucin receptor 3 (TIM-3, CD366), T-cell immunoglobulin and mucin domain-containing protein 4 (TIM-4), VISTA, sialic acid-binding Ig-like lectin (SIGLEC) 1 (SIGLEC1, CD169), SIGLEC2 (CD22), SIGLEC3 (CD33), SIGLEC5 (CD170), SIGLEC6 (CD328), SIGLEC7 (CD328), SIGLEC8, SIGLEC9 (CD329), SIGLEC10, TIGIT, PVR (CD155), lysosome associated membrane glycoprotein 1 (LAMP1, CD107a), lysosome associated membrane glycoprotein 2 (LAMP2, CD107b), lysosome associated membrane glycoprotein 3 (LAMP3, CD208), PECAM-1 (CD31), STAB-1, NRP2, CEACAM-1 (CD66a), TCR, VTCN1, NCR3LG1, B7-H7 (CD28H), IFNγ receptor 1, IFNγ receptor 2, CD2, CD4, lymphocyte function-associated antigen 3 (LFA-3, CD58), CD8, CD44, CEACAM3 (CD66d), CD96, IGSF2 (CD101), NECTIN1 (HVEC, CD111), NECTIN2 (CD112), NECTIN3 (CD113), DNAX accessory molecule 1 (DNAM-1, CD226), IL2RB (CD122), tyrosine-protein phosphatase no-receptor type substate 1 (SIRPa, CD172a), signal-regulatory protein beta-1 (SIRPB1, CD172b), signal-regulatory protein gamma (SIRPG, CD172g), OX-2 (CD200), OX-2R (CD200R), LAG3 (CD223), LAIR-1 (CD305), NKp30 (CD337), TWEAKR (CD266), CD3d, CD3e, CD3g, ITGAL (CD11a), ITGAM (CD11b), ITGAX (CD11c), ITGAD (CD11d), FCGR3A (CD16a), IL-4 receptor subunit alpha (IL4RA, CD124), IL-2 receptor subunit alpha (IL2RA, CD25), ITGB1 (CD29), CD30, low affinity immunoglobulin gamma Fc region receptor II-a (CD32a), low affinity immunoglobulin gamma Fc region receptor II-b (CD32b), complement receptor type I (CD35), leukosialin (CD43), CD44, receptor-type tyrosine-protein phosphatase C (CD45), membrane cofactor protein (CD46), integrin alpha-1 (CD49a), integrin alpha-2 (CD49b), integrin alpha-3 (CD49c), integrin alpha-4 (CD49d), integrin alpha-5 (CD49e), integrin alpha-6 (CD49f), intercellular adhesion molecule 3 (ICAM-3, CD50), intercellular adhesion molecule 1 (ICAM-1, CD54), ICAM-4 (CD242), integrin alpha V (ITGAV, CD51), integrin beta 3 (ITGB3, CD61), complement decay accelerating factor (CD55), neural adhesion molecule 1 (NCAM-1, CD56), CD62E, CD62L, CD62P, High affinity immunoglobulin gamma Fc receptor I (CD64), macrosialin (CD68), B-cell antigen receptor complex-associated protein alpha chain (CD79a), B-cell antigen receptor complex-associated protein beta chain (CD79b), CD83, leukocyte immunoglobulin-like receptor subfamily A members (CD85G, CD85H, CD85I), leukocyte immunoglobulin-like receptor subfamily B members (CD85A, CD85B, CD85C, CD85D, CD85F, CD85J, CD85K), Immunoglobulin alpha Fc receptor (CD89), CD91, CD93, FAS (CD95), T-cell surface protein tactile (CD96), CD99, semaphoring-D (CD100), immunoglobulin superfamily member 2 (CD101), intercellular adhesion molecule 2 (ICAM-2, CD102), integrin alpha-E (CD103), integrin beta-4 (ITGB4, CD104), endoglin (CD105), vascular cell adhesion protein 1 (VCAM1, CD106), thrombopoietin receptor (CD110), CD114, macrophage colony-stimulating factor 1 receptor (CSF1R, CD115), Granulocyte-macrophage colony-stimulating factor receptor subunit alpha (CSF2RA, CD116), mast/stem cell growth factor receptor Kit (CD117), leukemia inhibitory factor receptor (LIFR, CD118), interferon gamma receptor 1 (CD119), Tumor necrosis factor receptor superfamily member 1A (TNF-R1, CD120a), Tumor necrosis factor receptor superfamily member 1B (TNF-R2, CD120b), Interleukin-1 receptor type 1 (CD121a), Interleukin-1 receptor type 2 (CD121b), Interleukin-2 receptor subunit beta (CD122), Interleukin-3 receptor subunit alpha (IL3RA, CD123), Interleukin-4 receptor subunit alpha (IL4RA, CD124), Interleukin-5 receptor subunit alpha (IL5RA, CD125), Interleukin-6 receptor subunit alpha (IL6RA, CD126), Interleukin-6 receptor subunit beta (IL6ST, CD130), Interleukin-7 receptor subunit alpha (IL7RA, CD127), Interleukin-9 receptor (CD129), Cytokine receptor common subunit beta (CD131), Cytokine receptor common subunit gamma (CD132), CD135, macrophage stimulating protein receptor (CD136), syndecan-1 (CD138), Platelet-derived growth factor receptor alpha (PDGFRA, CD140a), Platelet-derived growth factor receptor beta (PDGFRB, CD140b), thrombomodulin (CD141), CD142, angiotensin converting enzyme (ACE, CD143), cadherin-5 (CD144), melanoma and adhesion molecule (MCAM, CD146), basigin (BSG, CD147), CD148, Signaling lymphocytic activation molecule (SLAM, CD150), SLAM family member 4 (SLAMF4, CD244), signaling lymphocytic activation molecule (SLAM) family member 5 (SLAM5, CD84), SLAM family member 6 (SLAMF6, CD352), SLAM family member 7 (SLAMF7, CD319), SLAM family member 8 (SLAMF8, CD353), SLAM family member 9 (SLAM9), Disintegrin and metalloproteinase domain-containing protein 8 (ADAM8, CD156a), Disintegrin and metalloproteinase domain-containing protein 17 (ADAM17, CD156b), Disintegrin and metalloproteinase domain-containing protein 10 (ADAM10, CD156c), P-selectin glycoprotein 1 (SELPLG, CD162), CD163, CD164, activated leukocyte cell adhesion molecule (ALCAM, CD166), epithelial discoidin domain containing receptor 1 (CD167a), discoidin domain containing receptor 2 (CD167b), neural cell adhesion molecule L1 (L1CAM, CD 171), CD180, endothelial protein C receptor (EPCR, CD201), angiopoietin-1 receptor (CD202b), lymphocyte antigen 75 (CD205), macrophage mannose receptor 1 (CD206), IL-10 receptor subunit alpha (IL10RA, CD210), IL-10 receptor subunit beta (IL10RB, CDw210b), IL-12 receptor subunit beta-1 (IL12RB1, CD212), IL-13 receptor subunit alpha-1 (CD213a1), IL-13 receptor subunit alpha-2 (CD213a2), IL-15 receptor subunit alpha (CD215), IL-17 receptor A (CD217), IL-18 receptor 1 (CD218a), IL-18 receptor accessory protein (CD218b), insulin receptor (CD220), insulin-like growth factor 1 receptor (CD221), cation-independent mannos-6phosphate receptor (CD222), mucin-1 (CD227), T-lymphocyte surface antigen Ly-9 (CD229), plexin-C1 (VESPR, CD232), glycophorin-A (CD235a), glycophorin-B (CD235b), basal cell adhesion molecule (CD239), CD246, T-cell surface glycoprotein CD3 zeta chain (CD247), endosialin (CD248), death receptor 3 (DR3, TNFRS25), death receptor 4 (DR4, CD261), death receptor 5 (DR5, CD262), decoy receptor 2 (DcR2, CD264), receptor activator of nuclear factor kappa-B (RANK, CD265), CD271, C-type mannose receptor 2 (CD280), Toll like receptor 1 (CD281), Toll like receptor 2 (CD282), Toll like receptor 3 (CD283), Toll like receptor 4 (CD284), Toll like receptor 6 (CD286), Toll like receptor 8 (CD288), Toll like receptor 9 (CD289), Toll like receptor 10 (CD290), bone morphogenic protein receptor type 1A (CD292), bone morphogenic protein receptor type ID (CwD293), leptin receptor (CD295), CD300a, CD300c, CD302, Neuropilin-1 (CD304), leukocyte-associated immunoglobulin-like receptor 1 (LIAR1, CD305), Fc receptor-like protein 1 (FcRL1, CD307a), Fc receptor-like protein 2 (FcRL2, CD307b), Fc receptor-like protein 3 (FcRL3, CD307c), Fc receptor-like protein 4 (FcRL4, CD307d), Fc receptor-like protein 5 (FcRL5, CD307e), vascular endothelial growth factor receptor 2 (VEGFR2, CD309), prostaglandin F2 receptor negative regulator (PTGFRN, CD315), immunoglobulin superfamily member 8 (IGSF8, CD316), CD320, platelet F11 receptor (F11R, CD321), junctional adhesion molecule B (JAM-B, CD322), cadherin-1 (CD324), cadherin-2 (CD325), epithelial cell adhesion molecule (CD326), fibroblast growth factor 1 (FGFR1, CD331), fibroblast growth factor 2 (FGFR2, CD332), fibroblast growth factor 3 (FGFR3, CD333), fibroblast growth factor 4 (FGFR4, CD334), natural cytotoxicity triggering receptor 1 (NCR1, CD335), natural cytotoxicity triggering receptor 2 (NCR2, CD336), natural cytotoxicity triggering receptor 3 (NCR3, CD337), triggering receptor expressing on myeloid cells 1 (TREM1, CD354), cytotoxic and regulatory T-cell molecule (CRTAM, CD355), tumor necrosis factor receptor superfamily member 21 (CD358), interleukin-21 receptor (IL21R, CD360), protein EVI2B (CD361), syndecan-2 (CD362), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), butyrophilin subfamily 3 member A1 (BTN3A1, CD277), butyrophilin subfamily 3 member A2 (BTN3A2), butyrophilin subfamily 2 member A1 (BTN2A1), butyrophilin like protein 8 (BTNL8), butyrophilin subfamily 1 member A1 (BTN1A1), lymphotoxin beta receptor (LTBR), DNAX-activation protein 10 (DAP10), TYRO protein tyrosine kinase-binding protein (DAP12), high affinity immunoglobulin epsilon receptor subunit gamma (FcεRIγ). Isoforms or fragments thereof, or the like known by one of ordinary skill in the art are also encompassed by the present invention.

### Type II Membrane Protein Vesicle Targeting Domain

In some embodiments of any of the aspects, the vesicle targeting domain comprises amino acid sequences from a type II membrane protein. Exemplary type II membrane proteins includes but are not limited to CD27L (CD70), CD40, CD40L (CD154), 4-1BB (CD137), 4-1BBL (CD137L), OX40L (CD252), glucocorticoid-induced TNF-related ligand (GITRL), LIGHT (CD258), TNF-related apoptosis inducing factor (TRAIL, CD253), CLEC7A (CD369), CD30L (CD153), TL1 (TNFSF15), FasL (CD178), NKG2 family ligands (NKG2A, B, C, D, E, F and H), B cell activating factor (BAFF, CD257), TNF-related weak inducer of apoptosis (TWEAK), RBAT (SLC3A1), ATPIB2, CD94, neprilysin (CD10), CD13, BLAST-2 (CD23), Dipeptidyl peptidase 4 (DPP4, ADCP2, CD26), CD38, CLEC2C (CD69), Transferrin receptor protein 1 (CD71), B-cell differentiation antigen Lyb-2 (CD72), HLA class II histocompatibility antigen gamma chain (CD74), CD75, CD77, natural killer cell antigen KLRD1 (CD94), NKG2-A/B-activating NK receptor (CD159a), NKG2-C (CD159c), killer cell lectin-like receptor subfamily B member 1 (CD161), galactoside alpha-(1,2)-fucosyltransferase 1 (FUT1, CD174), 3-galactosyl-N-acetylglucosamide 4-alpha-L-fucosyltransferase (FUT3), ectonucleotide pyrophosphatase/phosphodiesterase (ENPP) family member 1 (ENPP1, CD203a), ENPP family member 3 (ENPP3, CD203c), macrophage scavenger receptor types I and II (CD204), C-type lectin domain family 4 member K (langerin, CD207), dendritic cell-specific ICAM-3-grabbing non-integrin 1 (DC-SIGN, CD209), CD224, CD238, glutamyl aminopeptidase (CD249), receptor activator of nuclear factor kappa-B ligand (RANKL, CD254), CD298, DC-SIGN related protein (DC-SIGNR, CD299), C-type lectin domain family 10 member A (CLEC10A, CD301), C-type lectin domain family 4 member C (CLEC4C, CD303), NKG2-D type II integral membrane protein (KLRK1, CD314), bone marrow stromal antigen 2 (BST2, CD317), transmembrane and associated with src kinases (TRASK, CD318), protein jagged-1 (CD339), human epidermal growth factor 2 (HER2, CD340), C-type lectin domain family 4 member A (CLEC4A, CD367), C-type lectin domain family 4 member D (CLEC4D, CD368), C-type lectin domain family 7 member A (CLEC7A, CD369), C-type lectin domain family 9 member A (CLEC9A, CD370), C-type lectin domain family 12 member A (CLEC12A, CD371), SLC3A2 (CD98 heavy chain), tumor necrosis factor (TNF, TNF-alpha, TNFα), lymphotoxin-alpha (LTA, LT-α) also known as tumor necrosis factor ligand superfamily member 1 (TNF-beta, TNF-β), tumor necrosis family ligand superfamily member 3 also known as lymphotoxin beta (LTB, TNF-C, TNFγ), tumor necrosis factor ligand superfamily 15 (TL1A), and A proliferation-inducing ligand (APRIL, CD256). Isoforms or fragments thereof, or the like known by one of ordinary skill in the art are also included in the present invention.

In various embodiments, the Type II membrane protein vesicle targeting polypeptide or fragment thereof comprises the 4F2 heavy chain of human CD98 (4F2, h4F2 hc) encoded by the SLC3A2 gene. In various embodiments, the Type II transmembrane protein or fragment thereof comprises CD298 (ATB1B3). In various embodiments, the Type II transmembrane protein or fragment thereof comprises sequences belonging to the NKG2 family of C-type lectin-like receptors, for example NKG2D (encoded by the KLRK1 gene), or any isoform thereof, fragment thereof, or the like known by one of ordinary skill in the art. In some embodiments, the Type II membrane protein vesicle targeting domain may have biochemical affinity to EV resident proteins, e.g., CD81, CD63, CD9, ALIX, TSG101, CD98, CD298, MARCKS, PTGFRN, Lactadherin (MFGe8), ITGB1, EpCAM, MCAM, CD44, NCAM, ICAM. Forces that anchor EV targeting domains to the EV phospholipid bilayer may include, but are not limited to, electrostatic forces, affinity for EVs through protein-protein interactions with natively resident proteins, association or affinity for negatively or positively curved phospholipids, association or affinity for negatively or positively charged domains of resident membrane associated proteins, etc., or the like.

### Type III Membrane Protein Vesicle Targeting Domain

In some embodiments of any of the aspects, the vesicle targeting domain comprises amino acid sequences from a type III membrane protein. Exemplary type III membrane proteins include but are not limited to B cell activating factor (BAFFR, CD268), glycophorin-C(CD236), transmembrane activator and CAML interactor (TACI, CD267), B-cell maturation protein (BCM, CD269) Isoforms or fragments thereof, or the like known by one of ordinary skill in the art are also included in the invention.

### Multi-Pass Membrane Protein Vesicle Targeting Domain.

In some embodiments of any of the aspects, the vesicle targeting domain comprises amino acid sequences from a multi-pass membrane protein. In various embodiments, the multi-pass membrane protein vesicle targeting polypeptide or fragment thereof comprises LATI (CD98 light subunit encoded by the SLC7A5 gene). Exemplary multi-pass membrane proteins include but are not limited to Alpha-2A adrenergic receptor (A2AR, ADRA2A), adenosine receptor A2b (A2BR, ADORA2B), NOX2, LATI (SLC7A5 (CD98 light chain) and SLC7A2 (CD98 heavy chain)), CD39, CD47, PVRIG (CD112R), CD9, CD20, CD36, CD37, CD47, CD53, CD63, CD81, CD82, C5a receptor (CD88), CD92, CD97, prominin-1 (CD133), CD151, high affinity interleukin-8 receptor A (IL8RA, CXCR1, CD181), high affinity interleukin-8 receptor B (IL8RB, CXCR2, CD182), C—X—C chemokine receptor (CXCR) type 3 (CXCR3, CD183), CXCR4 (CD184), CXCR5 (CD185), CXCR6 (CD186), C—C chemokine (CCR) type 1 (CCR1, CD191), CCR2 (CD192), CCR3 (CD193), CCR4 (CD194), CCR5 (CD195), CCR6 (CD196), CCR7 (CD197), CCR8 (CDw189), CCR9 (CDw199), CD231, solute carrier family 4 member 1 (SLC4A1, CD233), Duffy antigen/chemokine receptor (DARC, CD234), blood group Rh (CE) polypeptide (CD240CE), blood group Rh (D) polypeptide (CD240D), ammonium transporter Rh type A (CD241), CD243, calcium signal-modulating cyclophilin ligand (CAMLG), prostaglandin D2 receptor 2 (PTGDR2, CD294), EGF-like module receptor 2 (CD312), CD338, frizzled-4 (CD344), frizzled-9 (CD349), frizzled-10 (CD350), sphingosine 1-phosphate receptor 1 (CD363), BAT1 encoded by the SLC7A9 gene, linker for activation of T-cell family member 2 (LAT2), or a fragment thereof.

In certain embodiments, the at least one multi-pass membrane protein is a tetraspanin selected from the group consisting of TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), TSPAN33, a fragment thereof, and combinations thereof.

### Lipid Anchored Membrane Protein Vesicle Targeting Domain

In various embodiments, the at least one vesicle targeting domain comprises amino acid sequences from a lipid anchored membrane protein or fragment thereof. In various embodiments, the lipid anchored membrane protein comprises a myristoylation or palmitoylation sequence, for example a myristoylation or palmitoylation tag from MARCKS (e.g. SEQ ID NO: 16). In various embodiments, the lipid anchored membrane sequence comprises a modified myristoylation or palmitoylation sequence, for example a modified myristoylation or palmitoylation tag from MARCKS.

In another embodiment of any of the aspects, the vesicle targeting domain is selected from those listed in Table 1A and 1B. In another embodiment of any of the aspects, the vesicle targeting domain is selected from the group consisting of: a Glycosylphosphatidylinositol (GPI) anchor, a fatty acetylation site, and a prenylation site. In another embodiment of any of the aspects, the vesicle targeting domain is a GPI anchor.

The extracellular vesicles provided herein further comprise at least one fusion polypeptide comprising a vesicle targeting domain. In various embodiments, the vesicle targeting domain provided herein binds to or anchors the fusion polypeptide provided to an extracellular vesicle, e.g., via targeting of the phospholipid bilayer membrane.

The extracellular vesicles provided herein further comprise at least one fusion polypeptide comprising a vesicle targeting domain. In various embodiments, the vesicle targeting domain provided herein is capable of binding or anchoring the fusion polypeptide provided herein to an extracellular vesicle, e.g., via targeting to the phospholipid bilayer membrane.

In various embodiments, the vesicle targeting domain is a GPI domain (i.e., GPI, GPI anchor), fatty acetylation site, or prenylation moiety. In some embodiments of any of the aspects, the vesicle targeting domain is a membrane anchoring domain from a GPI anchored membrane protein or fragment thereof. In various embodiments, a fragment of a GPI anchored membrane protein can be at least 75%, 80%, 85%, 90%, or 95% of a GPI anchored membrane protein. In certain embodiments, the GPI anchored membrane protein is selected from the group consisting of CD160, RGMB, CEACAM8 (CD66b, CD67), CEACAM6 (CD66c), CEACAM5 (CD66e), CD73, CD14, FCGR3B (CD16b), CD24, BLAST-1 (CD48), CAMPATH-1 (CD52), CD59, CD87, CD90, semaphorin-7A (CD108), CD109, bone marrow stromal cell antigen 1 (BST1, CD157), CD177, melanotransferrin (CD228), CD230, decoy receptor 1 (DcR1, CD263), CD296, CD297 isoforms thereof, fragments thereof, and combinations thereof. One of skill in the art can appreciate that the aforementioned refer to peptide or protein sites, wherein covalent lipid attachment supports embedding of the lipid in a cell membrane (i.e., phospholipid bilayer). Biochemical forces that anchor EV targeting domains to the EV phospholipid bilayer may include, but are not limited to, electrostatic forces, affinity for EVs through protein-protein interactions with natively resident proteins (e.g., CD81, CD63, CD9, ALIX, TSG101. CD98, CD298, MARCKS, PTGFRN, Lactadherin (MFGe8), ITGB1, EpCAM, MCAM, CD44, NCAM, ICAM), association or affinity for negatively or positively curved phospholipids, association or affinity for negatively or positively charged domains of resident membrane associated proteins, etc., or the like.

In various embodiments, the vesicle targeting domain is a membrane anchoring domain from a type I membrane protein or fragment thereof. In various embodiments, the vesicle targeting domain is a membrane anchoring domain from a type II membrane protein or fragment thereof. In various embodiments, the vesicle targeting domain is a membrane anchoring domain from a type III membrane protein or fragment thereof. In various embodiments, the vesicle targeting domain is a membrane anchoring domains from a multi-pass membrane protein or fragment thereof.

In various embodiments, the vesicle targeting domain comprises a membrane anchoring domain from a type I membrane protein or fragment thereof. In various embodiments, the vesicle targeting domain comprises a membrane anchoring domain from a type II membrane protein or fragment thereof. In various embodiments, the vesicle targeting domain comprises a membrane anchoring domain from a type III membrane protein or fragment thereof. In various embodiments, the vesicle targeting domain comprises one or more membrane anchoring domains from a multi-pass membrane protein or fragment thereof.

In various embodiments, a fragment of a membrane anchoring domain or a fragment of a multi-pass transmembrane domain can be at least 75%, 80%, 85%, 90%, or 95% of a membrane anchoring domain or at least 75%, 80%, 85%, 90%, or 95% of a multi-pass transmembrane domain.

Additional non-limiting examples of vesicle targeting domains (also referred to membrane targeting domain or membrane anchoring domain) that can be used and their properties are further described in detail, e.g., Alberts B, Johnson A, Lewis J, et al., Molecular Biology of the Cell, 4th edition, New York: Garland Science, 2002. Membrane Proteins, ncbi.nlm.nih.gov/books/NBK26878/; Marilyn D. Resh, Fatty acylation of proteins: new insights into membrane targeting of myristoylated and palmitoylated proteins. Biochimica et Biophysica Acta (BBA)-Molecular Cell Research. Volume 1451, Issue 1, 12 Aug. 1999, Pages 1-16, doi.org/10.1016/S0167-4889 (99) 00075-0; Ann Apolloni, et. al., H-ras but Not K-ras Traffics to the Plasma Membrane through the Exocytic Pathway, Molecular and Cellular Biology April 2000, 20 (7) 2475-2487, DOI: 10.1128/MCB.20.7.2475-2487.2000; Rosie Dawaliby et. al., Phosphatidylethanolamine Is a Key Regulator of Membrane Fluidity in Eukaryotic Cells, Membrane Biology, VOLUME 291, ISSUE 7, doi.org/10.1074/jbc.M115.706523; R. J. Deschenes, Protein Palmitoylation, Encyclopedia of Biological Chemistry (Second Edition), Academic Press, 2013, Pages 645-647, ISBN 9780123786319, doi.org/10.1016/B978-0-12-378630-2.00022-0.; Charuta C. Palsuledesai and Mark D. Distefano, Protein Prenylation: Enzymes, Therapeutics, and Biotechnology Applications, ACS Chemical Biology 2015 10 (1), 51-62, DOI: 10.1021/cb500791f; Hung M E, Leonard J N. Stabilization of exosome-targeting peptides via engineered glycosylation, J Biol Chem, 2015 Mar. 27; 290 (13): 8166-72, doi: 10.1074/jbc.M114.621383; Udenwobele Daniel Ikenna, et. al., Myristoylation: An Important Protein Modification in the Immune Response, Frontiers in Immunology, Vol: 8, 2017, DOI=10.3389/fimmu.2017.00751; Kinoshita Taroh 2020Biosynthesis and biology of mammalian GPI-anchored proteins Open Biol. 10190290, doi.org/10.1098/rsob.190290, Martin D D, Beauchamp E, Berthiaume L G. Post-translational myristoylation: Fat matters in cellular life and death. Biochimie. 2011 January; 93 (1): 18-31. doi: 10.1016/j.biochi.2010.10.018. Epub 2010 Nov. 5. PMID: 21056615, the contents of which are incorporated herein by reference in their entireties.

In some embodiments, the fusion polypeptide comprises one, two, three, four, five, six, seven, eight, nine, ten, or more membrane anchoring domains from one or more membrane proteins. For example, the fusion polypeptides provided herein can comprise a 4-1BBL multi-effector signaling domain, a myristoylation and palmitoylation (Myr/Palm) lipid anchoring motif (e.g. SEQ ID NO: 18) and 4F2 (CD98 heavy chain) or fragments thereof (e.g. SEQ ID NO: 12). Another example, the fusion polypeptide provided herein can comprise or consists of a 4-1BBL signaling multi-effector domain, a 4F2 (CD98 heavy chain) membrane anchoring domain, and a lipid anchoring palmitoylation and myristoylation (Myr/Palm) motif. Another example, the fusion polypeptides provided herein can comprise Myr/Palm membrane anchoring motif and a CD298 membrane protein sequence or fragments thereof (e.g. SEQ ID NO: 14). Another example, the fusion polypeptides provided herein can comprise Myr/Palm membrane anchoring motif and a membrane anchoring domain from CD298 membrane protein or fragments thereof. In various embodiments, the vesicle targeting domains may be separated by one or more linkers.

In some embodiments, the vesicle targeting domain is a prenylated protein or fragment thereof. Prenylated proteins are proteins that have at least one prenylation site. Prenylation occurs when a 15-carbon or 20-carbon, farnesyl or geranylgeranyl isoprenoid, respectively, is covalently bound via a thioether bond to a cysteine at or near the carboxy terminus of a protein. In general, a prenylation site comprises an amino acid sequence CAAX, wherein C represents cysteine, A represents an aliphatic amino acid (glycine, alanine, valine, leucine, or isoleucine), and X represents alanine, methionine, serine, leucine, or glutamine.

In some embodiments, the vesicle targeting domain is a fatty acylated protein or fragment thereof. Fatty acylated proteins are proteins that have been modified post-translationally by covalent attachment of one or more fatty acids, generally with a saturated fatty acid that comprises 14-carbon (e.g., myristic acid) via myristoylation (Myr) or 16-carbons (e.g. palmitic acid) via palmitoylation (Palm). For example, proteins destined to become myristoylated begin with the amino acids Met-Gly-X-X-X) followed by a serine or threonine at position 6 and lysine or arginine at position 7 and/or 8 wherein X can be any amino acid. The methionine is removed and a myristate is linked to the glycine via an amide bond. Palmitoylation herein means a posttranslational covalent attachment of fatty acids (e.g., palmitic acid) to cysteine (S-palmitoylation), serine and/or threonine (O-palmitoylation), and to the amino group of lysine (N-palmitoylation) of proteins.

Palmitoylated proteins may be acylated by attachment of a thioester linkage to a sulfhydryl group of cysteine, or via a palmitate linked to the amino group of an N-terminal cysteine. Palmitoylation sites may be present near the N- or C-terminus of a protein.

In some embodiments, the vesicle targeting domain is a glycosylphosphatidylinositol (GPI) anchor. A glycosylphosphatidylinositol (GPI) anchor (“GPI anchor”) or “GPI vesicle targeting domain” are used interchangeably and refer to a method of stably anchoring a protein to an outer leaflet (e.g., exterior layer of a phospholipid bilayer) of a cell membrane. A GPI anchor comprises a glycan, a phosphoethanolamine linker, a phospholipid tail, and may be modified by various glycan sidechains. The glycan core comprises phosphoinositol, glucosamine, and mannose residues wherein said mannose residues may be modified for example with phosphoethanolamine or carbohydrates. The phosphoethanolamine is amide-bonded to the carboxyl terminus of a protein during the process of GPI attachment. In some embodiments, the vesicle targeting domain may have affinity to EV resident proteins, e.g., CD81, CD63, CD9, ALIX, TSG101, CD98, CD298, MARCKS, PTGFRN, Lactadherin (MFGe8), ITGB1, EpCAM, MCAM, CD44, NCAM, ICAM.

In some embodiments, the vesicle targeting domain may have affinity to extracellular vesicle resident proteins, e.g., tetraspanins; TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), TSPAN33, or the like known by one of ordinary skill in the art.

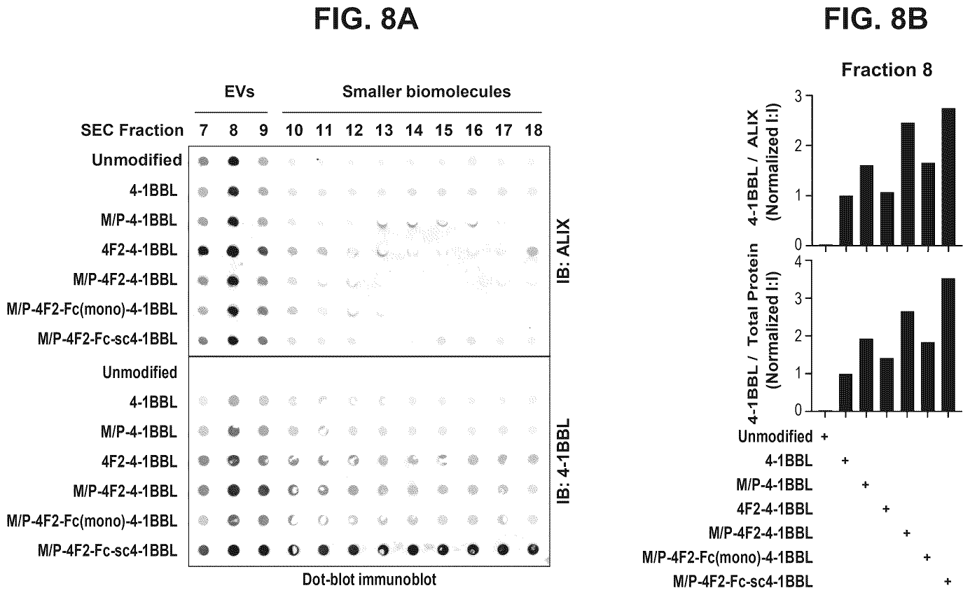

Vesicle targeting domains can include a sequence for one or more myristoylation and/or palmitoylation (Myr/Palm) sites linked by one or more linkers to a transmembrane domain from 4F2 (CD98). For example, the myristoylation sequence from the MARCKS protein may be modified to encode for one or more myristoylation and palmitoylation sites, wherein the modified MARCKS protein sequence is linked by one or more linkers to a transmembrane domain from 4F2 via a covalent peptide bond. A Myr/Palm followed by the 4F2 transmembrane domain can improve loading of the fusion proteins provided herein when compared with 4F2 transmembrane domain alone or Myr/Palm alone (FIG. 8).

Non-limiting examples of vesicle targeting domains that enhance fusion polypeptide structure and function on the extracellular vesicles are provided in Table 1A and 1B (below). Fusion polypeptides presented herein may comprise a vesicle targeting domain wherein the vesicle targeting domain is at least one membrane anchoring domain from at least one membrane protein. Table 1A lists non-limiting examples of Type I membrane proteins and proteins comprising a GPI anchor domain, wherein bolded sequences are exemplary Type I membrane anchoring domains or GPI membrane anchor domains, respectively. Type I membrane compatible vesicle targeting domains are membrane anchoring domains that anchor proteins to a membrane (e.g., an EV membrane) in an orientation that of a Type I membrane protein or a protein with a GPI membrane anchor. Table 1B lists non-limiting examples of Type II membrane proteins, wherein bolded sequences are exemplary type II membrane anchoring domains compatible with type II membrane protein fusion polypeptides. Type II membrane compatible vesicle targeting domains are membrane anchoring domains that anchor proteins to a membrane (e.g., an EV membrane) in an orientation that of a Type II membrane protein.

TABLE 1A
Type I Membrane Compatible Vesicle Targeting Domain

| Exosome | |
|---|---|
| Targeting | Nucleic Acid Sequence (SEQ ID NO:) |
| Domain | Amino Acid Sequence (SEQ ID NO:) |
| Human CD55 | >NM_000574.5 Homo sapiens CD55 molecule (Cromer blood group) |
| (DAF) | (CD55), transcript variant 1, mRNA |
| Glycosylphos | CTGCTTACTGCAACTCGCTCCGGCCGCTGGGCGTAGCTGCGACTCGGCGGAGTC |
| phatidylinositol | CCGGCGGCGCGTCCTTGTTCTAACCCGGCGCGCCATGACCGTCGCGCGGCCGAG |
| (GPI) | CGTGCCCGCGGCGCTGCCCCTCCTCGGGGAGCTGCCCCGGCTGCTGCTGCTGGT |
| | GCTGTTGTGCCTGCCGGCCGTGTGGGGTGACTGTGGCCTTCCCCCAGATGTACC |
| | TAATGCCCAGCCAGCTTTGGAAGGCCGTACAAGTTTTCCCGAGGATACTGTAAT |
| | AACGTACAAATGTGAAGAAAGCTTTGTGAAAATTCCTGGCGAGAAGGACTCAG |
| | TGATCTGCCTTAAGGGCAGTCAATGGTCAGATATTGAAGAGTTCTGCAATCGTA |
| | GCTGCGAGGTGCCAACAAGGCTAAATTCTGCATCCCTCAAACAGCCTTATATCA |
| | CTCAGAATTATTTTCCAGTCGGTACTGTTGTGGAATATGAGTGCCGTCCAGGTT |
| | ACAGAAGAGAACCTTCTCTATCACCAAAACTAACTTGCCTTCAGAATTTAAAAT |
| | GGTCCACAGCAGTCGAATTTTGTAAAAAGAAATCATGCCCTAATCCGGGAGAA |
| | ATACGAAATGGTCAGATTGATGTACCAGGTGGCATATTATTTGGTGCAACCATC |
| | TCCTTCTCATGTAACACAGGGTACAAATTATTTGGCTCGACTTCTAGTTTTTGTC |
| | TTATTTCAGGCAGCTCTGTCCAGTGGAGTGACCCGTTGCCAGAGTGCAGAGAAA |
| | TTTATTGTCCAGCACCACCACAAATTGACAATGGAATAATTCAAGGGGAACGTG |
| | ACCATTATGGATATAGACAGTCTGTAACGTATGCATGTAATAAAGGATTCACCA |
| | TGATTGGAGAGCACTCTATTTATTGTACTGTGAATAATGATGAAGGAGAGTGGA |
| | GTGGCCCACCACCTGAATGCAGAGGAAAATCTCTAACTTCCAAGGTCCCACCA |
| | ACAGTTCAGAAACCTACCACAGTAAATGTTCCAACTACAGAAGTCTCACCAACT |
| | TCTCAGAAAACCACCACAAAAACCACCACACCAAATGCTCAAGCAACACGGAG |
| | TACACCTGTTTCCAGGACAACCAAGCATTTTCATGAAACAACCCCAAATAAAG |
| | GAAGTGGAACCACTTCAGGTACTACCCGTCTTCTATCTGGGCACACGTGTT |
| | TCACGTTGACAGGTTTGCTTGGGACGCTAGTAACCATGGGCTTGCTGACTT |
| | AGCCAAAGAAGAGTTAAGAAGAAAATACACACAAGTATACAGACTGTTCCTAG |
| | TTTCTTAGACTTATCTGCATATTGGATAAAATAAATGCAATTGTGCTCTTCATTT |
| | AGGATGCTTTCATTGTCTTTAAGATGTGTTAGGAATGTCAACAGAGCAAGGAGA |
| | AAAAAGGCAGTCCTGGAATCACATTCTTAGCACACCTACACCTCTTGAAAATAG |
| | AACAACTTGCAGAATTGAGAGTGATTCCTTTCCTAAAAGTGTAAGAAAGCATA |
| | GAGATTTGTTCGTATTTAGAATGGGATCACGAGGAAAAGAGAAGGAAAGTGAT |
| | TTTTTTCCACAAGATCTGTAATGTTATTTCCACTTATAAAGGAAATAAAAAATG |
| | AAAAACATTATTTGGATATCAAAAGCAAATAAAAACCCAATTCAGTCTCTTCTA |
| | AGCAAAATTGCTAAAGAGAGATGAACCACATTATAAAGTAATCTTTGGCTGTA |
| | AGGCATTTTCATCTTTCCTTCGGGTTGGCAAAATATTTTAAAGGTAAAACATGC |
| | TGGTGAACCAGGGGTGTTGATGGTGATAAGGGAGGAATATAGAATGAAAGACT |
| | GAATCTTCCTTTGTTGCACAAATAGAGTTTGGAAAAAGCCTGTGAAAGGTGTCT |
| | TCTTTGACTTAATGTCTTTAAAAGTATCCAGAGATACTACAATATTAACATAAG |
| | AAAAGATTATATATTATTTCTGAATCGAGATGTCCATAGTCAAATTTGTAAATC |
| | TTATTCTTTTGTAATATTTATTTATATTTATTTATGACAGTGAACATTCTGATTTT |
| | ACATGTAAAACAAGAAAAGTTGAAGAAGATATGTGAAGAAAAATGTATTTTTC |
| | CTAAATAGAAATAAATGATCCCATTTTTTGGTATCATGTAGTATGTGAAATTTA |
| | TTCTTAAACGTGACTACTTTATTTCTAAATAAGAAATTCCCTACCTGCTTCCTAC |
| | AAGCAGTTCAGAATGCCATGCCTTGGTTGTCCTAGTGTGAATAATTTTCAGCTA |
| | CTTTAAAATTATATTGTACTTTCTCAAGCATGTCATATCCTTTCCTATTAGAGTA |
| | TCTATATTACTTGTTACTGATTTACCTGAAGGCAATCTGATTAATTTCTAGGTTT |
| | TTACCATATTCTTGTCATCTTGCCAATTACATTTTAAGTGTTAGACTAGACTAAG |
| | ATGTACTAGTTGTATAGAATATAACTAGATTTATTATGGCAATGTTTATTTTGTC |
| | ATTTTGCTTCATCTGTTTTGTTGTTGAAGTACTTTAAATTTCATACGTTCATGGC |
| | ATTTCACTGTAAAGACTTTAATGTGTATTTCTTAAAATAAAACTTTTTTTCCTCC |
| | TTAA (SEQ ID NO: 1) |
| | >NP_000565.1 complement decay-accelerating factor isoform 1 |
| | preproprotein [Homo sapiens] |
| | MTVARPSVPAALPLLGELPRLLLLVLLCLPAVWGDCGLPPDVPNAQPALEGRTSFP |
| | EDTVITYKCEESFVKIPGEKDSVICLKGSQWSDIEEFCNRSCEVPTRLNSASLKQPYI |
| | TQNYFPVGTVVEYECRPGYRREPSLSPKLTCLQNLKWSTAVEFCKKKSCPNPGEIR |
| | NGQIDVPGGILFGATISFSCNTGYKLFGSTSSFCLISGSSVQWSDPLPECREIYCPAPP |
| | QIDNGIIQGERDHYGYRQSVTYACNKGFTMIGEHSIYCTVNNDEGEWSGPPPECRG |
| | KSLTSKVPPTVQKPTTVNVPTTEVSPTSQKTTTKTTTPNAQATRSTPVSRTTKHFHE |
| | TTPNKGSGTTSGTTRLLSGHTCFTLTGLLGTLVTMGLLT (SEQ ID NO: 2) |
| Human CD59 | >NM_203330.2 Homo sapiens CD59 molecule (CD59 blood group) |
| Glycosylphos | (CD59), transcript variant 1, mRNA |
| phatidylinositol | GGGGCCGGGGGGCGGAGCCTTGCGGGCTGGAGCGAAAGAATGCGGGGGCTGA |
| (GPI) | GCGCAGAAGCGGCTCGAGGCTGGAAGAGGATCTTGGGCGCCGCCAGTCTCTCT |
| | CTGTTGCCCAAGCTGGAGTGCAGTGGCACAGTCTTGGCTCACTGCAACCTCCAC |
| | CTCCTGGGTGCAAGCGATTCTCGTGTCTCAGCCTCTCAAGTAGCTGGGATTACA |
| | GTCTTTAGCACCAGTTGGTGTAGGAGTTGAGACCTACTTCACAGTAGTTCTGTG |
| | GACAATCACAATGGGAATCCAAGGAGGGTCTGTCCTGTTCGGGCTGCTGCTCGT |
| | CCTGGCTGTCTTCTGCCATTCAGGTCATAGCCTGCAGTGCTACAACTGTCCTAA |
| | CCCAACTGCTGACTGCAAAACAGCCGTCAATTGTTCATCTGATTTTGATGCGTG |
| | TCTCATTACCAAAGCTGGGTTACAAGTGTATAACAAGTGTTGGAAGTTTGAGCA |
| | TTGCAATTTCAACGACGTCACAACCCGCTTGAGGGAAAATGAGCTAACGTACTA |
| | CTGCTGCAAGAAGGACCTGTGTAACTTTAACGAACAGCTTGAAAATGGTGG |
| | GACATCCTTATCAGAGAAAACAGTTCTTCTGCTGGTGACTCCATTTCTGGC |
| | AGCAGCCTGGAGCCTTCATCCCTAAGTCAACACCAGGAGAGCTTCTCCCAAA |
| | CTCCCCGTTCCTGCGTAGTCCGCTTTCTCTTGCTGCCACATTCTAAAGGCTTGAT |
| | ATTTTCCAAATGGATCCTGTTGGGAAAGAATAAAATTAGCTTGAGCAACCTGGC |
| | TAAGATAGAGGGGCTCTGGGAGACTTTGAAGACCAGTCCTGTTTGCAGGGAAG |
| | CCCCACTTGAAGGAAGAAGTCTAAGAGTGAAGTAGGTGTGACTTGAACTAGAT |
| | TGCATGCTTCCTCCTTTGCTCTTGGGAAGACCAGCTTTGCAGTGACAGCTTGAG |
| | TGGGTTCTCTGCAGCCCTCAGATTATTTTTCCTCTGGCTCCTTGGATGTAGTCAG |
| | TTAGCATCATTAGTACATCTTTGGAGGGTGGGGCAGGAGTATATGAGCATCCTC |
| | TCTCACATGGAACGCTTTCATAAACTTCAGGGATCCCGTGTTGCCATGGAGGCA |
| | TGCCAAATGTTCCATATGTGGGTGTCAGTCAGGGACAACAAGATCCTTAATGCA |
| | GAGCTAGAGGACTTCTGGCAGGGAAGTGGGGAAGTGTTCCAGATAGCAGGGCA |
| | TGAAAACTTAGAGAGGTACAAGTGGCTGAAAATCGAGTTTTTCCTCTGTCTTTA |
| | AATTTTATATGGGCTTTGTTATCTTCCACTGGAAAAGTGTAATAGCATACATCA |
| | ATGGTGTGTTAAAGCTATTTCCTTGCCTTTTTTTTATTGGAATGGTAGGATATCT |
| | TGGCTTTGCCACACACAGTTACAGAGTGAACACTCTACTACATGTGACTGGCAG |
| | TATTAAGTGTGCTTATTTTAAATGTTACTGGTAGAAAGGCAGTTCAGGTATGTG |
| | TGTATATAGTATGAATGCAGTGGGGACACCCTTTGTGGTTACAGTTTGAGACTT |
| | CCAAAGGTCATCCTTAATAACAACAGATCTGCAGGGGTATGTTTTACCATCTGC |
| | ATCCAGCCTCCTGCTAACTCCTAGCTGACTCAGCATAGATTGTATAAAATACCT |
| | TTGTAACGGCTCTTAGCACACTCACAGATGTTTGAGGCTTTCAGAAGCTCTTCT |
| | AAAAAATGATACACACCTTTCACAAGGGCAAACTTTTTCCTTTTCCCTGTGTATT |
| | CTAGTGAATGAATCTCAAGATTCAGTAGACCTAATGACATTTGTATTTTATGAT |
| | CTTGGCTGTATTTAATGGCATAGGCTGACTTTTGCAGATGGAGGAATTTCTTGA |
| | TTAATGTTGAAAAAAAACCCTTGATTATACTCTGTTGGACAAACCGAGTGCAAT |
| | GAATGATGCTTTTCTGAAAATGAAATATAACAAGTGGGTGAATGTGGTTATGGC |
| | CGAAAAGGATATGCAGTATGCTTAATGGTAGCAACTGAAAGAAGACATCCTGA |
| | GCAGTGCCAGCTTTCTTCTGTTGATGCCGTTCCCTGAACATAGGAAAATAGAAA |
| | CTTGCTTATCAAAACTTAGCATTACCTTGGTGCTCTGTGTTCTCTGTTAGCTCAG |
| | TGTCTTTCCTTACATCAATAGGTTTTTTTTTTTTTTTTTGGCCTGAGGAAGTACTG |
| | ACCATGCCCACAGCCACCGGCTGAGCAAAGAAGCTCATTTCATGTGAGTTCTAA |
| | GGAATGAGAAACAATTTTGATGAATTTAAGCAGAAAATGAATTTCTGGGAACT |
| | TTTTTGGGGGGGGGGGGGTGGGGAATTCAGCCACACTCCAGAAAGCCAGGAGT |
| | CGACAGTTTTGGAAGCCTCTCTCAGGATTGAGATTCTAGGATGAGATTGGCTTA |
| | CTGCTATCTTGTGTCATGTACCCACTTTTTGGCCAGACTACACTGGGAAGAAGG |
| | TAGTCCTCTAAAGCAAAATCTGAGTGCCACTAAATGGGGAGATGGGGCTGTTA |
| | AGCTGTCCAAATCAACAAGGGTCATATAAATGGCCTTAAACTTTGGGGTTGCTT |
| | TCTGCAAAAAGTTGCTGTGACTCATGCCATAGACAAGGTTGAGTGCCTGGACCC |
| | AAAGGCAATACTGTAATGTAAAGACATTTATAGTACTAGGCAAACAGCACCCC |
| | AGGTACTCCAGGCCCTCCTGGCTGGAGAGGGCTGTGGCAATAGAAAATTAGTG |
| | CCAACTGCAGTGAGTCAGCCTAGGTTAAATAGAGAGTGTAAGAGTGCTGGACA |
| | GGAACCTCCACCCTCATGTCACATTTCTTCAATGTGACCCTTCTGGCCCCTCTCC |
| | TCCTGACAGCGGAACAATGACTGCCCCGATAGGTGAGGCTGGAGGAAGAATCA |
| | GTCCTGTCCTTGGCAAGCTCTTCACTATGACAGTAAAGGCTCTCTGCCTGCTGC |
| | CAAGGCCTGTGACTTTCTAACCTGGCCTCACGCTGGGTAAGCTTAAGGTAGAGG |
| | TGCAGGATTAGCAAGCCCACCTGGCTACCAGGCCGACAGCTACATCCTCCAACT |
| | GACCCTGATCAACGAAGAGGGATTCATGTGTCTGTCTCAGTTGGTTCCAAATGA |
| | AACCAGGGAGCAGGGGAGTTAGGAATCGAACACCAGTCATGCCTACTGGCTCT |
| | CTGCTCGAGAGCCAATACCCTGTGCCCTCCACTCATCTGGATTTACAGGAACTG |
| | TCATAGTGTTCAGTATTGGGTGGTGATAAGCCCATTGGATTGTCCCCTTGGGGG |
| | GATGAGCTAGGGGTGCAAGGAACACCTGATGAGTAGATAAGTGGAGCTCATGG |
| | TATTTCCTGAAAGATGCTAATCTATTTGCCAAACTTGGTCTTGAATGTACTGGG |
| | GGCTTCAAGGTATGGGTATATTTTTCTTGTGTCCTTGCAGTTAGCCCCCATGTCT |
| | TATGTGTGTCCTGAAAAAATAAGAGCCTGCCCAAGACTTTGGGCCTCTTGACAG |
| | AATTAACCACTTTTATACATCTGAGTTCTCTTGGTAAGTTCTTTAGCAGTGTTCA |
| | AAGTCTACTAGCTCGCATTAGTTTCTGTTGCTGCCAACAGATCTGAACTAATGC |
| | TAACAGATCCCCCTGAGGGATTCTTGATGGGCTGAGCAGCTGGCTGGAGCTAGT |
| | ACTGACTGACATTCATTGTGATGAGGGCAGCTTTCTGGTACAGGATTCTAAGCT |
| | CTATGTTTTATATACATTTTCATCTGTACTTGCACCTCACTTTACACAAGAGGAA |
| | ACTATGCAAAGTTAGCTGGATCGCTCAAGGTCACTTAGGTAAGTTGGCAAGTCC |
| | ATGCTTCCCACTCAGCTCCTCAGGTCAGCAAGTCTACTTCTCTGCCTATTTTGTA |
| | TACTCTCTTTAATATGTGCCTAGCTTTGGAAAGTCTAGAATGGGTCCCTGGTGC |
| | CTTTTTACTTTGAAGAAATCAGTTTCTGCCTCTTTTTGGAAAAGAAAACAAAGT |
| | GCAATTGTTTTTTACTGGAAAGTTACCCAATAGCATGAGGTGAACAGGACGTAG |
| | TTAGGCCTTCCTGTAAACAGAAAATCATATCAAAACACTATCTTCCCATCTGTT |
| | TCTCAATGCCTGCTACTTCTTGTAGATATTTCATTTCAGGAGAGCAGCAGTTAA |
| | ACCCGTGGATTTTGTAGTTAGGAACCTGGGTTCAAACCCTCTTCCACTAATTGG |
| | CTATGTCTCTGGACAAGTTTTTTTTTTTTTTTTTTTTTAAACCCTTTCTGAACTTT |
| | CACTTTCTATGTCTACCTCAAAGAATTGTTGTGAGGCTTGAGATAATGCATTTGT |
| | AAAGGGTCTGCCAGATAGGAAGATGCTAGTTATGGATTTACAAGGTTGTTAAG |
| | GCTGTAAGAGTCTAAAACCTACAGTGAATCACAATGCATTTACCCCCACTGACT |
| | TGGACATAAGTGAAAACTAGCCAGAAGTCTCTTTTTCAAATTACTTACAGGTTA |
| | TTCAATATAAAATTTTTGTAATGGATAATCTTATTTATCTAAACTAAAGCTTCCT |
| | GTTTATACACACTCCTGTTATTCTGGGATAAGATAAATGACCACAGTACCTTAA |
| | TTTCTAGGTGGGTGCCTGTGATGGTTCATTGTAGGTAAGGACATTTTCTCTTTTT |
| | CAGCAGCTGTGTAGGTCCAGAGCCTCTGGGAGAGGAGGGGGGTAGCATGCACC |
| | CAGCAGGGGACTGAACTGGGAAACTCAAGGTTCTTTTTACTGTGGGGTAGTGA |
| | GCTGCCTTTCTGTGATCGGTTTCCCTAGGGATGTTGCTGTTCCCCTCCTTGCTAT |
| | TCGCAGCTACATACAACGTGGCCAACCCCAGTAGGCTGATCCTATATATGATCA |
| | GTGCTGGTGCTGACTCTCAATAGCCCCACCCAAGCTGGCTATAGGTTTACAGAT |
| | ACATTAATTAGGCAACCTAAAATATTGATGCTGGTGTTGGTGTGACATAATGCT |
| | ATGGCCAGAACTGAAACTTAGAGTTATAATTCATGTATTAGGGTTCTCCAGAGG |
| | GACAGAATTAGTAGGATATATGTATATATGAAAGGGAGGTTATTAGGGAGAAC |
| | TGGCTCCCACAGTTAGAAGGCGAAGTCGCACAATAGGCCGTCTGCAAGCTGGG |
| | TTAGAGAGAAGCCAGTAGTGGCTCAGCCTGAGTTCAAAAACCTCAAAACTGGG |
| | GAAGCTGACAGTGCAGCCAGCCTTCAGTCTGTGGCCAAAGGCCCAAGAGCCCC |
| | TGGCAACCAACCCACTGGTGCAAGTCCTAGATTCCAAAGGCTGAAGAACCTGG |
| | AGTCTGATGTCCAAGAGCAGGAAGAGTGGAAGAAAGCCAGAAGACTCAGCAA |
| | ACAAGGTAGACAGTGTCTACCACCATAGTGGCCATACCAAAGAGGCTACCGAT |
| | TCCTTCCTGCTACCTGGATCCCTGAAGTTGCCCTGGTCTCTGCACCTTCTAAACC |
| | TAGTTCTTAAGAGCTTTCCATTACATGAGCTGTCTCAAAGCCCTCCAATAAATT |
| | CTCAGTGTAAGCTTCTGTTGCTTGTGGACAGAAAATTCTGACAGACCTACCCTA |
| | TAAGTGTTACTGTCAGGATAACATGAGAACGCACAACAGTAAGTGGTCACTAA |
| | GTGTTAGCTACGGTTATTTTGCCCAAGGTAGCATGGCTAGTTGATGCCGGTTGA |
| | TGGGGCTTAAACCCAGCTCCCTCATCTTCCAGGCCTCTGTACTCCCTATTCCACT |
| | AAACTACCTCTCAGGTTTATTTTTTTAAATTCTTACTCTGCAAGTACATAGGACC |
| | ACATTTACCTGGGAAAACAAGAATAAAGGCTGCTCTGCATTTTTTAGAAACTTT |
| | TTTGAAAGGGAGATGGGAATGCCTGCACCCCCAAGTCCAGACCAACACAATGG |
| | TTAATTGAGATGAATAATAAAGGAAAGACTGTTCTGGGCTTCCCAGAATAGCTT |
| | GGTCCTTAAATTGTGGCACAAACAACCTCCTGTCAGAGCCAGCCTCCTGCCAGG |
| | AAGAGGGGTAGGAGACTAGAGGCCGTGTGTGCAGCCTTGCCCTGAAGGCTAGG |
| | GTGACAATTTGGAGGCTGTCCAAACACCCTGGCCTCTAGAGCTGGCCTGTCTAT |
| | TTGAAATGCCGGCTCTGATGCTAATCGGCGACCCTCAGGCAAGTTACTTAACCT |
| | TACATGCCTCAGTTTTCTCATCTGGAAAATGAGAACCCTAGGTTTAGGGTTGTT |
| | AGAAAAGTTAAATGAGTTAAGACAAGTGCCTGGGACACAGTAGCCTCTTGTGT |
| | GTGTTTATCATTATGTCCTCAGCAGGTCGTAGAAGCAGCTTCTCAGGTGTGAGG |
| | CTGGCGCGATTATCTGGAGTGGGTTGGGTTTTCTAGGATGGACCCCCTGCTGCA |
| | TTTTCCTCATTCATCCACCAGGGCTTAATGGGGAATCAAGGAATCCATGTGTAA |
| | CTGTATAATAACTGTAGCCACACTCCAATGACCACCTACTAGTTGTCCCTGGCA |
| | CTGCTTATACATATGTCCATCAAATCAATCCTATGAAGTAGATACTGTCTTCATT |
| | TTATAGATCAGAGACAATTGGGGTTCAGAGAGCTGATGTGATTTTCCCAGGGTC |
| | ACAGAGAGTCCCAGATTCAGGCACAACTCTTGTATTCCAAGACACAACCACTA |
| | CATGTCCAAAGGCTGCCCAGAGCCACCGGGCACGGCAAATTGTGACATATCCC |
| | TAAAGAGGCTGAGCACCTGGTCAGGATCTGATGGCTGACAGTGTGTCCAGATG |
| | CAGAGCTGGAGTGGGGGAGGGGAAGGGGGGCTCCTTGGGACAGAGAAGGCTT |
| | TCTGTGCTTTCTCTGAAGGGAGCAGTCTGAGGACCAAGGGAACCCGGCAAACA |
| | GCACCTCAGGTACTCCAGGCCCTCCTGGCTGGAGAGGGCTGTGGCAATGGAAA |
| | ATTAGTGCCAACTGCAATGAGTCAGCCTCGGTTAAATAGAGAGTGAAGAATGC |
| | TGGACAGGAACCTCCACCCTCATGTCACATTTCTTCAGTGTGACCCTTCTGGCC |
| | CCTCTCCTCCTGACAGCGGAACAATGACTGCCCCGATAGGTGAGGCTGGAGGA |
| | AGAATCAGTCCTGTCCTTGGCAAGCTCTTCACTATGACAGTAAAGGCTCTCTGC |
| | CTGCTGCCAAGGCCTGTGACTTTCTAACCTGGCCTCACGCTGGGTAAGCTTAAG |
| | GTAGAGGTGCAGGATTAGCAAGCCCACCTGGCTACCAGGCCGACAGCTACATC |
| | TTTCAACTGACCCTGATCAACGAAGAGGGACTTGTGTCTCTCAGTTGGTTCCAA |
| | ATGAAACCAGGGAGCAGGGGCGTTAGGAAGCTCCAACAGGATGGTACTTAATG |
| | GGGCATTTGAGTGGAGAGGTAGGTGACATAGTGCTTTGGAGCCCAGGGAGGGA |
| | AAGGTTCTGCTGAAGTTGAATTCAAGACTGTTCTTTCATCACAAACTTGAGTTT |
| | CCTGGACATTTGTTTGCAGAAACAACCGTAGGGTTTTGCCTTAACCTCGTGGGT |
| | TTATTATTACCTCATAGGGACTTTGCCTCCTGACAGCAGTTTATGGGTGTTCATT |
| | GTGGCACTTGAGTTTTCTTGCATACTTGTTAGAGAAACCAAGTTTGTCATCAAC |
| | TTCTTATTTAACCCCCTGGCTATAACTTCATGGATTATGTTATAATTAAGCCATC |
| | CAGAGTAAAATCTGTTTAGATTATCTTGGAGTAAGGGGGAAAAAATCTGTAATT |
| | TTTTCTCCTCAACTAGATATATACATAAAAAATGATTGTATTGCTTCATTTAAAA |
| | AATATAACGCAAAATCTCTTTTCCTTCTAAAAAAAAAAAAAAAAAA (SEQ ID |
| | NO: 3) |
| | >NP_976075.1 CD59 glycoprotein preproprotein [Homo sapiens] |
| | MGIQGGSVLFGLLLVLAVFCHSGHSLQCYNCPNPTADCKTAVNCSSDFDACLITKA |
| | GLQVYNKCWKFEHCNFNDVTTRLRENELTYYCCKKDLCNFNEQLENGGTSLSE |
| | KTVLLLVTPFLAAAWSLHP (SEQ ID NO: 4) |
| Human C1C2 | NM_005928.4 Homo sapiens milk fat globule-EGF factor 8 |
| from MFGE8 | protein (MFGE8), transcript variant 1, mRNA |
| | AGAACCCCGCGGGGTCTGAGCAGCCCAGCGTGCCCATTCCAGCGCCCGCGTCC |
| | CCGCAGCATGCCGCGCCCCCGCCTGCTGGCCGCGCTGTGCGGCGCGCTGCTCTG |
| | CGCCCCCAGCCTCCTCGTCGCCCTGGATATCTGTTCCAAAAACCCCTGCCACAA |
| | CGGTGGTTTATGCGAGGAGATTTCCCAAGAAGTGCGAGGAGATGTCTTCCCCTC |
| | GTACACCTGCACGTGCCTTAAGGGCTACGCGGGCAACCACTGTGAGACGAAAT |
| | GTGTCGAGCCACTGGGCCTGGAGAATGGGAACATTGCCAACTCACAGATC |
| | GCCGCCTCGTCTGTGCGTGTGACCTTCTTGGGTTTGCAGCATTGGGTCCCG |
| | GAGCTGGCCCGCCTGAACCGCGCAGGCATGGTCAATGCCTGGACACCCAG |
| | CAGCAATGACGATAACCCCTGGATCCAGGTGAACCTGCTGCGGAGGATGT |
| | GGGTAACAGGTGTGGTGACGCAGGGTGCCAGCCGCTTGGCCAGTCATGAG |
| | TACCTGAAGGCCTTCAAGGTGGCCTACAGCCTTAATGGACACGAATTCGAT |
| | TTCATCCATGATGTTAATAAAAAACACAAGGAGTTTGTGGGTAACTGGAAC |
| | AAAAACGCGGTGCATGTCAACCTGTTTGAGACCCCTGTGGAGGCTCAGTA |
| | CGTGAGATTGTACCCCACGAGCTGCCACACGGCCTGCACTCTGCGCTTTGA |
| | GCTACTGGGCTGTGAGCTGAACGGATGCGCCAATCCCCTGGGCCTGAAGA |
| | ATAACAGCATCCCTGACAAGCAGATCACGGCCTCCAGCAGCTACAAGACCT |
| | GGGGCTTGCATCTCTTCAGCTGGAACCCCTCCTATGCACGGCTGGACAAG |
| | CAGGGCAACTTCAACGCCTGGGTTGCGGGGAGCTACGGTAACGATCAGTG |
| | GCTGCAGGTGGACCTGGGCTCCTCGAAGGAGGTGACAGGCATCATCACCC |
| | AGGGGGCCCGTAACTTTGGCTCTGTCCAGTTTGTGGCATCCTACAAGGTTG |
| | CCTACAGTAATGACAGTGCGAACTGGACTGAGTACCAGGACCCCAGGACT |
| | GGCAGCAGTAAGATCTTCCCTGGCAACTGGGACAACCACTCCCACAAGAA |
| | GAACTTGTTTGAGACGCCCATCCTGGCTCGCTATGTGCGCATCCTGCCTGT |
| | AGCCTGGCACAACCGCATCGCCCTGCGCCTGGAGCTGCTGGGCTGTTAGT |
| | GGCCACCTGCCACCCCCAGGTCTTCCTGCTTTCCATGGGCCCGCTGCCTCTTGGC |
| | TTCTCAGCCCCTTTAAATCACCATAGGGCTGGGGACTGGGGAAGGGGAGGGTG |
| | TTCAGAGGCAGCACCACCACACAGTCACCCCTCCCTCCCTCTTTCCCACCCTCC |
| | ACCTCTCACGGGCCCTGCCCCAGCCCCTAAGCCCCGTCCCCTAACCCCCAGTCC |
| | TCACTGTCCTGTTTTCTTAGGCACTGAGGGATCTGAGTAGGTCTGGGATGGACA |
| | GGAAAGGGCAAAGTAGGGCGTGTGGTTTCCCTGCCCCTGTCCGGACCGCCGAT |
| | CCCAGGTGCGTGTGTCTCTGTCTCTCCTAGCCCCTCTCTCACACATCACATTCCC |
| | ATGGTGGCCTCAAGAAAGGCCCGGAAGCGCCAGGCTGGAGATAACAGCCTCTT |
| | GCCCGTCGGCCCTGCGTCGGCCCTGGGGTACCATGTGGCCACAACTGCTGTGGC |
| | CCCCTGTCCCCAAGACACTTCCCCTTGTCTCCCTGGTTGCCTCTCTTGCCCCTTG |
| | TCCTGAAGCCCAGCGACACAGAAGGGGGTGGGGGGGGTCTATGGGGAGAAAG |
| | GGAGCGAGGTCAGAGGAGGGCATGGGTTGGCAGGGTGGGCGTTTGGGGCCCTC |
| | TATGCTGGCTTTTCACCCCAGAGGACACAGGCAGCTTCCAAAATATATTTATCT |
| | TCTTCACGGGAA (SEQ ID NO: 5) |
| | >NP_005919.2 lactadherin isoform a preproprotein [Homo sapiens] |
| | MPRPRLLAALCGALLCAPSLLVALDICSKNPCHNGGLCEEISQEVRGDVFPSYTCTC |
| | LKGYAGNHCETKCVEPLGLENGNIANSQIAASSVRVTFLGLQHWVPELARLNR |
| | AGMVNAWTPSSNDDNPWIQVNLLRRMWVTGVVTQGASRLASHEYLKAFKVA |
| | YSLNGHEFDFIHDVNKKHKEFVGNWNKNAVHVNLFETPVEAQYVRLYPTSCH |
| | TACTLRFELLGCELNGCANPLGLKNNSIPDKQITASSSYKTWGLHLFSWNPSY |
| | ARLDKQGNFNAWVAGSYGNDQWLQVDLGSSKEVTGIITQGARNFGSVQFVAS |
| | YKVAYSNDSANWTEYQDPRTGSSKIFPGNWDNHSHKKNLFETPILARYVRILP |
| | VAWHNRIALRLELLGC (SEQ ID NO: 6) |
| Transmembrane | >NM_001769.4 Homo sapiens CD9 molecule (CD9), |
| domain 2 or | transcript variant 1, mRNA |
| transmembrane | AGCCGCCTGCATCTGTATCCAGCGCCAGGTCCCGCCAGTCCCAGCTGCGCGCGC |
| domain 4 | CCCCCAGTCCCGCACCCGTTCGGCCCAGGCTAAGTTAGCCCTCACCATGCCGGT |
| from Human CD9 | CAAAGGAGGCACCAAGTGCATCAAATACCTGCTGTTCGGATTTAACTTCATCTT |
| | CTGGCTTGCCGGGATTGCTGTCCTTGCCATTGGACTATGGCTCCGATTCGACTCT |
| | CAGACCAAGAGCATCTTCGAGCAAGAAACTAATAATAATAATTCCAGCTTCTA |
| | CACAGGAGTCTATATTCTGATCGGAGCCGGCGCCCTCATGATGCTGGTGG |
| | GCTTCCTGGGCTGCTGCGGGGCTGTGCAGGAGTCCCAGTGCATGCTGGGAC |
| | TGTTCTTCGGCTTCCTCTTGGTGATATTCGCCATTGAAATAGCTGCGGCCATCTG |
| | GGGATATTCCCACAAGGATGAGGTGATTAAGGAAGTCCAGGAGTTTTACAAGG |
| | ACACCTACAACAAGCTGAAAACCAAGGATGAGCCCCAGCGGGAAACGCTGAA |
| | AGCCATCCACTATGCGTTGAACTGCTGTGGTTTGGCTGGGGGCGTGGAACAGTT |
| | TATCTCAGACATCTGCCCCAAGAAGGACGTACTCGAAACCTTCACCGTGAAGTC |
| | CTGTCCTGATGCCATCAAAGAGGTCTTCGACAATAAATTCCACATCATCGGCGC |
| | AGTGGGCATCGGCATTGCCGTGGTCATGATATTTGGCATGATCTTCAGTATGAT |
| | CTTGTGCTGTGCTATCCGCAGGAACCGCGAGATGGTCTAGAGTCAGCTTACATC |
| | CCTGAGCAGGAAAGTTTACCCATGAAGATTGGTGGGATTTTTTGTTTGTTTGTTT |
| | TGTTTTGTTTGTTGTTTGTTGTTTGTTTTTTTGCCACTAATTTTAGTATTCATTCTG |
| | CATTGCTAGATAAAAGCTGAAGTTACTTTATGTTTGTCTTTTAATGCTTCATTCA |
| | ATATTGACATTTGTAGTTGAGCGGGGGGTTTGGTTTGCTTTGGTTTATATTTTTT |
| | CAGTTGTTTGTTTTTGCTTGTTATATTAAGCAGAAATCCTGCAATGAAAGGTACT |
| | ATATTTGCTAGACTCTAGACAAGATATTGTACATAAAAGAATTTTTTTGTCTTTA |
| | AATAGATACAAATGTCTATCAACTTTAATCAAGTTGTAACTTATATTGAAGACA |
| | ATTTGATACATAATAAAAAATTATGACAATGTCCTGGA (SEQ ID NO: 7) |
| | >NP_001760.1 CD9 antigen isoform 1 [Homo sapiens] |
| | MPVKGGTKCIKYLLFGFNFIFWLAGIAVLAIGLWLRFDSQTKSIFEQETNNNNSSFY |
| | TGVYILIGAGALMMLVGFLGCCGAVQESQCMLGLFFGFLLVIFAIEIAAAIWGYS |
| | HKDEVIKEVQEFYKDTYNKLKTKDEPQRETLKAIHYALNCCGLAGGVEQFISDICP |
| | KKDVLETFTVKSCPDAIKEVFDNKFHIIGAVGIGIAVVMIFGMIFSMILCCAIRRNR |
| | EMV (SEQ ID NO: 8) |
| CD9tm2 | >CD9tm2, nucleic acid sequence |
| | TTCTACACAGGAGTCTATATTCTGATCGGAGCCGGCGCCCTCATGATGCTGGTG |
| | GGCTTCCTGGGCTGCTGCGGGGCTGTGCAGGAGTCCCAGTGC |
| | (SEQ ID NO: 9) |
| | >CD9tm2, amino acid sequence |
| | FYTGVYILIGAGALMMLVGFLGCCGAVQESQC (SEQ ID NO: 10) |

TABLE 1B
Type II Membrane Compatible Vesicle Targeting Domain

| Exosome | |
|---|---|
| Targeting | Nucleic Acid Sequence (SEQ ID NO:) |
| Domain | Amino Acid Sequence (SEQ ID NO:) |
| Human 4F2 | >NM_002394.6 Homo sapiens solute carrier family 3 |
| (CD98 Heavy | member 2 (SLC3A2), transcript variant 3, mRNA |
| Chain) | GCATTGCGGCTTGGTTTTCTCACCCAGTGCATGTGGCAGGAGCGGTGAGATCAC |
| Bold: amino | TGCCTCACGGCGATCCTGGACTGACGGTCACGACTGCCTACCCTCTAACCCTGT |
| acids 2-105 | TCTGAGCTGCCCCTTGCCCACACACCCCAAACCTGTGTGCAGGATCCGCCTCCA |
| | TGGAGCTACAGCCTCCTGAAGCCTCGATCGCCGTCGTGTCGATTCCGCGCCAGT |
| | TGCCTGGCTCACATTCGGAGGCTGGTGTCCAGGGTCTCAGCGCGGGGGACGACT |
| | CAGAGTTGGGGTCTCACTGTGTTGCCCAGACTGGTCTCGAACTCTTGGCCTCAG |
| | GTGATCCTCTTCCCTCAGCTTCCCAGAATGCCGAGATGATAGAGACGGGGTCTG |
| | ACTGTGTTACCCAGGCTGGTCTTCAACTCTTGGCCTCAAGTGATCCTCCTGCCTT |
| | AGCTTCCAAGAATGCTGAGGTTACAGGCACCATGAGCCAGGACACCGAGGTG |
| | GATATGAAGGAGGTGGAGCTGAATGAGTTAGAGCCCGAGAAGCAGCCGAT |
| | GAACGCGGCGTCTGGGGCGGCCATGTCCCTGGCGGGAGCCGAGAAGAAT |
| | GGTCTGGTGAAGATCAAGGTGGCGGAAGACGAGGCGGAGGCGGCAGCCG |
| | CGGCTAAGTTCACGGGCCTGTCCAAGGAGGAGCTGCTGAAGGTGGCAGGC |
| | AGCCCCGGCTGGGTACGCACCCGCTGGGCACTGCTGCTGCTCTTCTGGCTC |
| | GGCTGGCTCGGCATGCTTGCTGGTGCCGTGGTCATAATCGTGCGAGCGCCGCGT |
| | TGTCGCGAGCTACCGGCGCAGAAGTGGTGGCACACGGGCGCCCTCTACCGCAT |
| | CGGCGACCTTCAGGCCTTCCAGGGCCACGGCGCGGGCAACCTGGCGGGTCTGA |
| | AGGGGCGTCTCGATTACCTGAGCTCTCTGAAGGTGAAGGGCCTTGTGCTGGGTC |
| | CAATTCACAAGAACCAGAAGGATGATGTCGCTCAGACTGACTTGCTGCAGATC |
| | GACCCCAATTTTGGCTCCAAGGAAGATTTTGACAGTCTCTTGCAATCGGCTAAA |
| | AAAAAGAGCATCCGTGTCATTCTGGACCTTACTCCCAACTACCGGGGTGAGAAC |
| | TCGTGGTTCTCCACTCAGGTTGACACTGTGGCCACCAAGGTGAAGGATGCTCTG |
| | GAGTTTTGGCTGCAAGCTGGCGTGGATGGGTTCCAGGTTCGGGACATAGAGAA |
| | TCTGAAGGATGCATCCTCATTCTTGGCTGAGTGGCAAAATATCACCAAGGGCTT |
| | CAGTGAAGACAGGCTCTTGATTGCGGGGACTAACTCCTCCGACCTTCAGCAGAT |
| | CCTGAGCCTACTCGAATCCAACAAAGACTTGCTGTTGACTAGCTCATACCTGTC |
| | TGATTCTGGTTCTACTGGGGAGCATACAAAATCCCTAGTCACACAGTATTTGAA |
| | TGCCACTGGCAATCGCTGGTGCAGCTGGAGTTTGTCTCAGGCAAGGCTCCTGAC |
| | TTCCTTCTTGCCGGCTCAACTTCTCCGACTCTACCAGCTGATGCTCTTCACCCTG |
| | CCAGGGACCCCTGTTTTCAGCTACGGGGATGAGATTGGCCTGGATGCAGCTGCC |
| | CTTCCTGGACAGCCTATGGAGGCTCCAGTCATGCTGTGGGATGAGTCCAGCTTC |
| | CCTGACATCCCAGGGGCTGTAAGTGCCAACATGACTGTGAAGGGCCAGAGTGA |
| | AGACCCTGGCTCCCTCCTTTCCTTGTTCCGGCGGCTGAGTGACCAGCGGAGTAA |
| | GGAGCGCTCCCTACTGCATGGGGACTTCCACGCGTTCTCCGCTGGGCCTGGACT |
| | CTTCTCCTATATCCGCCACTGGGACCAGAATGAGCGTTTTCTGGTAGTGCTTAA |
| | CTTTGGGGATGTGGGCCTCTCGGCTGGACTGCAGGCCTCCGACCTGCCTGCCAG |
| | CGCCAGCCTGCCAGCCAAGGCTGACCTCCTGCTCAGCACCCAGCCAGGCCGTG |
| | AGGAGGGCTCCCCTCTTGAGCTGGAACGCCTGAAACTGGAGCCTCACGAAGGG |
| | CTGCTGCTCCGCTTCCCCTACGCGGCCTGACTTCAGCCTGACATGGACCCACTA |
| | CCCTTCTCCTTTCCTTCCCAGGCCCTTTGGCTTCTGATTTTTCTCTTTTTTAAAAA |
| | CAAACAAACAAACTGTTGCAGATTATGAGTGAACCCCCAAATAGGGTGTTTTCT |
| | GCCTTCAAATAAAAGTCACCCCTGCATGGTGAA (SEQ ID NO: 11) |
| | >NP_002385.3 4F2 cell-surface antigen heavy chain isoform |
| | c [Homo sapiens] |
| | MELQPPEASIAVVSIPRQLPGSHSEAGVQGLSAGDDSELGSHCVAQTGLELLAS |
| | GDPLPSASQNAEMIETGSDCVTQAGLQLLASSDPPALASKNAEVTGTMSQDTE |
| | VDMKEVELNELEPEKQPMNAASGAAMSLAGAEKNGLVKIKVAEDEAEAAAA |
| | AKFTGLSKEELLKVAGSPGWVRTRWALLLLFWLGWLGMLAGAVVIIVRAPR |
| | CRELPAQKWWHTGALYRIGDLQAFQGHGAGNLAGLKGRLDYLSSLKVKGLVLGP |
| | IHKNQKDDVAQTDLLQIDPNFGSKEDFDSLLQSAKKKSIRVILDLTPNYRGENSWFS |
| | TQVDTVATKVKDALEFWLQAGVDGFQVRDIENLKDASSFLAEWQNITKGFSEDRL |
| | LIAGTNSSDLQQILSLLESNKDLLLTSSYLSDSGSTGEHTKSLVTQYLNATGNRWCS |
| | WSLSQARLLTSFLPAQLLRLYQLMLFTLPGTPVFSYGDEIGLDAAALPGQPMEAPV |
| | MLWDESSFPDIPGAVSANMTVKGQSEDPGSLLSLFRRLSDQRSKERSLLHGDFHAF |
| | SAGPGLFSYIRHWDQNERFLVVLNFGDVGLSAGLQASDLPASASLPAKADLLLSTQ |
| | PGREEGSPLELERLKLEPHEGLLLRFPYAA |
| | (SEQ ID NO: 12) |
| Human | >NM_001679.4 Homo sapiens ATPase Na+/K+ transporting |
| CD298 | subunit beta 3 (ATP1B3), mRNA |
| (ATB1B3) | AGTCGGCTCGAGTACTCCCCGTAACGAGGAGGTGTTCTCGGCCGTCCCACCCTT |
| Domain | CACTGCCGTCTCCGGGCTGCGCCGCCGGAGCCGGGACGCGCCTCCGCAGCCCTC |
| Bold: amino | GCCGCCTCCATCCCCGCGGCCGCAGCTCCTCTCGCCGTCCGCGCGCACACCATG |
| acids 2-57 | ACGAAGAACGAGAAGAAGTCCCTCAACCAGAGCCTGGCCGAGTGGAAGCT |
| | CTTCATCTACAACCCGACCACCGGAGAATTCCTGGGGCGCACCGCCAAGA |
| | GCTGGGGTTTGATCTTGCTCTTCTACCTAGTTTTTTATGGGTTCCTGGCTG |
| | CACTCTTCTCATTCACGATGTGGGTTATGCTTCAGACTCTCAACGATGAGGTT |
| | CCAAAATACCGTGACCAGATTCCTAGCCCAGGACTCATGGTTTTTCCAAAACCA |
| | GTGACCGCATTGGAATATACATTCAGTAGGTCTGATCCAACTTCGTATGCAGGG |
| | TACATTGAAGACCTTAAGAAGTTTCTAAAACCATATACTTTAGAAGAACAGAA |
| | GAACCTCACAGTCTGTCCTGATGGAGCACTTTTTGAACAGAAGGGTCCAGTTTA |
| | TGTTGCATGTCAGTTTCCTATTTCATTACTTCAAGCATGCAGTGGTATGAATGAT |
| | CCTGATTTTGGCTATTCTCAAGGAAACCCTTGTATTCTTGTGAAAATGAACAGA |
| | ATAATTGGATTAAAGCCTGAAGGAGTGCCAAGGATAGATTGTGTTTCAAAGAA |
| | TGAAGATATACCAAATGTAGCAGTTTATCCTCATAATGGAATGATAGACTTAAA |
| | ATATTTCCCATATTATGGGAAAAAACTGCATGTTGGGTATCTACAGCCATTGGT |
| | TGCTGTTCAGGTCAGCTTTGCTCCTAACAACACTGGGAAAGAAGTAACAGTTGA |
| | GTGCAAGATTGATGGATCAGCCAACCTAAAAAGTCAGGATGATCGTGACAAGT |
| | TTTTGGGACGAGTTATGTTCAAAATCACAGCACGTGCATAGTATGAGTAGGATA |
| | TCTCCACAGAGTAAATGTTGTGTTGTCTGTCTTCATTTTGTAACAGCTGGACCTT |
| | CCATTCTAGAATTATGAGACCACCTTGGAGAAAGGTGTGTGGTACATGACATTG |
| | GGTTACATCATAACGTGCTTCCAGATCATAGTGTTCAGTGTCCTCTGAAGTAAC |
| | TGCCTGTTGCCTCTGCTGCCCTTTGAACCAGTGTACAGTCGCCAGATAGGGACC |
| | GGTGAACACCTGATTCCAAACATGTAGGATGGGGGTCTTGTCCTCTTTTTATGT |
| | GGTTTAATTGCCAAGTGTCTAAAGCTTAATATGCCGTGCTATGTAAATATTTTAT |
| | GGATATAACAACTGTCATATTTTGATGTCAACAGAGTTTTAGGGATAAAATGGT |
| | ACCCGGCCAACATCAAGTGACTTTATAGCTGCAAGAAATGTGGTATGTGGAGA |
| | AGTTCTGTATGTGAGGAAGGAAAAAAAGAAAATAAAAGTGTGTTTGAAAAATA |
| | TTATCTTGGGTTCTTTGTAAAATTTATTTTTTACATGCTGAATTAGCCTCGATCTT |
| | TTTGATTAAGAGCACAAACTTTTTTTTGTAAAACATGTAAAAAAAAAAACTGGG |
| | ATTAATTTTTAGTGTTGGAACTGCCTCTTATTTTAGGCTGTAGATAAAATAGCAT |
| | TTTTAGGTTAGCCAGTGTGACTATGCACCTAATTTTTTATGAGATTAAATTCATA |
| | AGACTTAATTTGTACAATAGTTTGTGAAATATCTTGTTACTGCTTTTATTTAGCA |
| | GACTGTGGACTGTAATAAAGTATATAAATTGTGAAATATAAAAACTTGGAACTT |
| | ATTCAAAGCTTCAAAGCAAA (SEQ ID NO: 13) |
| | >NP_001670.1 sodium/potassium-transporting ATPase subunit |
| | beta-3 [Homo sapiens] |
| | MTKNEKKSLNQSLAEWKLFIYNPTTGEFLGRTAKSWGLILLFYLVFYGFLAAL |
| | FSFTMWVMLQTLNDEVPKYRDQIPSPGLMVFPKPVTALEYTFSRSDPTSYAGYIED |
| | LKKFLKPYTLEEQKNLTVCPDGALFEQKGPVYVACQFPISLLQACSGMNDPDFGYS |
| | QGNPCILVKMNRIIGLKPEGVPRIDCVSKNEDIPNVAVYPHNGMIDLKYFPYYGKK |
| | LHVGYLQPLVAVQVSFAPNNTGKEVTVECKIDGSANLKSQDDRDKFLGRVMFKIT |
| | ARA (SEQ ID NO: 14) |
| Myr tag in | >NM_002356.7 Homo sapiens myristoylated alanine rich protein |
| Human | kinase C substrate (MARCKS), mRNA |
| MARCKS | GCACTTGGGCGTTGGACCCCGCATCTTATTAGCAACCAGGGAGATTTCTCCATT |
| Bold: amino | TTCCTCTTGTCTACAGTGCGGCTACAAATCTGGGATTTTTTTATTACTTCTTTTTT |
| acids 1-8 | TTTCGAACTACACTTGGGCTCCTTTTTTTGTGCTCGACTTTTCCACCCTTTTTCCC |
| (M/P tag) | TCCCTCCTGTGCTGCTGCTTTTTGATCTCTTCGACTAAAATTTTTTTATCCGGAGT |
| | GTATTTAATCGGTTCTGTTCTGTCCTCTCCACCACCCCCACCCCCCTCCCTCCGG |
| | TGTGTGTGCCGCTGCCGCTGTTGCCGCCGCCGCTGCTGCTGCTGCTCGCCCCGTC |
| | GTTACACCAACCCGAGGCTCTTTGTTTCCCCTCTTGGATCTGTTGAGTTTCTTTG |
| | TTGAAGAAGCCAGCATGGGTGCCCAGTTCTCCAAGACCGCAGCGAAGGGAG |
| | AAGCCGCCGCGGAGAGGCCTGGGGAGGCGGCTGTGGCCTCGTCGCCTTCCAAA |
| | GCGAACGGACAGGAGAATGGCCACGTGAAGGTAAACGGCGACGCTTCGCCCGC |
| | GGCCGCCGAGTCGGGCGCCAAGGAGGAGCTGCAGGCCAACGGCAGCGCCCCG |
| | GCCGCCGACAAGGAGGAGCCCGCGGCCGCCGGGAGCGGGGCGGCGTCGCCCTC |
| | CGCGGCCGAGAAAGGTGAGCCGGCCGCCGCCGCTGCCCCCGAGGCCGGGGCCA |
| | GCCCGGTAGAGAAGGAGGCCCCCGCGGAAGGCGAGGCTGCCGAGCCCGGCTCG |
| | CCCACGGCCGCGGAGGGAGAGGCCGCGTCGGCCGCCTCCTCGACTTCTTCGCCC |
| | AAGGCCGAGGACGGGGCCACGCCCTCGCCCAGCAACGAGACCCCGAAAAAAA |
| | AAAAGAAGCGCTTTTCCTTCAAGAAGTCTTTCAAGCTGAGCGGCTTCTCCTTCA |
| | AGAAGAACAAGAAGGAGGCTGGAGAAGGCGGTGAGGCTGAGGCGCCCGCTGC |
| | CGAAGGCGGCAAGGACGAGGCCGCCGGGGGCGCAGCTGCGGCCGCCGCCGAG |
| | GCGGGCGCGGCCTCCGGGGAGCAGGCAGCGGCGCCGGGCGAGGAGGCGGCAG |
| | CGGGCGAGGAGGGGGCGGCGGGTGGCGACCCGCAGGAGGCCAAGCCCCAGGA |
| | GGCCGCTGTCGCGCCAGAGAAGCCGCCCGCCAGCGACGAGACCAAGGCCGCCG |
| | AGGAGCCCAGCAAGGTGGAGGAGAAAAAGGCCGAGGAGGCCGGGGCCAGCGC |
| | CGCCGCCTGCGAGGCCCCCTCCGCCGCCGGGCCCGGCGCGCCCCCGGAGCAGG |
| | AGGCAGCCCCCGCGGAGGAGCCCGCGGCCGCCGCAGCCTCGTCAGCCTGCGCA |
| | GCCCCCTCACAGGAGGCCCAGCCCGAGTGCAGTCCAGAAGCCCCCCCAGCGGA |
| | GGCGGCAGAGTAAAAGAGCAAGCTTTTGTGAGATAATCGAAGAACTTTTCTCC |
| | CCCGTTTGTTTGTTGGAGTGGTGCCAGGTACTGGTTTTGGAGAACTTGTCTACA |
| | ACCAGGGATTGATTTTAAAGATGTCTTTTTTTATTTTACTTTTTTTTAAGCACCA |
| | AATTTTGTTGTTTTTTTTTTTTCTCCCCTCCCCACAGATCCCATCTCAAATCATTC |
| | TGTTAACCACCATTCCAACAGGTCGAGGAGAGCTTAAACACCTTCTTCCTCTGC |
| | CTTGTTTCTCTTTTATTTTTTATTTTTTCGCATCAGTATTAATGTTTTTGCATACTT |
| | TGCATCTTTATTCAAAAGTGTAAACTTTCTTTGTCAATCTATGGACATGCCCATA |
| | TATGAAGGAGATGGGTGGGTCAAAAAGGGATATCAAATGAAGTGATGGGGTCA |
| | CAATGGGGAAATTGAAGTGGTGCATAACATTGCCAAAATAGTGTGCCACTAGA |
| | AATGGTGTAAAGGCTGTCTTTTTTTTTTTTTTAAAAGAAAAGTTATTACCATGTA |
| | TTTTGTGAGGCAGGTTTACAACACTACAAGTCTTGAGTTAAGAAGGAAAGAGG |
| | AAAAAAGAAAAAACACCAATACCCAGATTTAAAAAAAAAAAAACGATCATAG |
| | TCTTAGGAGTTCATTTAAACCATAGGAACTTTTCACTTATCTCATGTTAGCTGTA |
| | CCAGTCAGTGATTAAGTAGAACTACAAGTTGTATAGGCTTTATTGTTTATTGCT |
| | GGTTTATGACCTTAATAAAGTGTAATTATGTATTACCAGCAGGGTGTTTTTAAC |
| | TGTGACTATTGTATAAAAACAAATCTTGATATCCAGAAGCACATGAAGTTTGCA |
| | ACTTTCCACCCTGCCCATTTTTGTAAAACTGCAGTCATCTTGGACCTTTTAAAAC |
| | ACAAATTTTAAACTCAACCAAGCTGTGATAAGTGGAATGGTTACTGTTTATACT |
| | GTGGTATGTTTTTGATTACAGCAGATAATGCTTTCTTTTCCAGTCGTCTTTGAGA |
| | ATAAAGGAAAAAAAATCTTCAGATGCAATGGTTTTGTGTAGCATCTTGTCTATC |
| | ATGTTTTGTAAATACTGGAGAAGCTTTGACCAATTTGACTTAGAGATGGAATGT |
| | AACTTTGCTTACAAAAATTGCTATTAAACTCCTGCTTAAGGTGTTCTAATTTTCT |
| | GTGAGCACACTAAAAGCGAAAAATAAATGTGAATAAAATGTACAAATTTGTTG |
| | TGTTTTTTTATGTTCTAATAATACTGAGACTTCTAGGTCTTAGGTTAATTTTTAG |
| | GAAGATCTTGCATGCCATCAGGAGTAAATTTTATTGTGGTTCTTAATCTGAAGT |
| | TTTCAAGCTCTGAAATTCATAATCCGCAGTGTCAGATTACGTAGAGGAAGATCT |
| | TACAACATTTCCATGTCAAATCTGTTACCATTTATTGGCATTTAGTTTTCATTTA |
| | AGAATTGAACATAATTATTTTTATTGTAGCTATATAGCATGTCAGATTAAATCA |
| | TTTACAACAAAAGGGGTGTGAACCTAAGACTATTTAAATGTCTTATGAGAAAAT |
| | TTCATAAAGCCATTCTCTTGTCATTCAGGTCCAGAAACAAATTTTAAACTGAGT |
| | GAGAGTCTATAGAATCCATACTGCAGATGGGTCATGAAATGTGACCAAATGTG |
| | TTTCAAAAATTGATGGTGTATTACCTGCTATTGTAATTGCTTAGTGCTTGGCTAA |
| | TTTCCAAATTATTGCATAATATGTTCTACCTTAAGAAAACAGGTTTATGTAACA |
| | AAGTAATGGTGTTGAATGGATGATGTCAGTTCATGGGCCTTTAGCATAGTTTTA |
| | AGCATCCTTTTTTTTTTTTTTTTTTGAAAGTGTGTTAGCATCTTGTTACTCAAAGG |
| | ATAAGACAGACAATAATACTTCACTGAATCTTAATAATCTTTACTAGTTTACCT |
| | CCTCTGCTCTTTGCCACCCGATAACTGGATATCTTTTCCTTCAAAGGACCCTAAA |
| | CTGATTGAAATTTAAGATATGTATCAAAAACATTATTTCATTTAATGCACATCT |
| | GTTTTGCTGTTTTTGAGCAGTGTGCAGTTTAGGGTTCATGATAAATCATTGAACC |
| | ACATGTGTAACAACTGAATGCCAAATCTTAAACTCATTAGAAAAATAACAAATT |
| | AGGTTTTGACACGCATTCTTAATTGGAATAATGGATCAAAAATAGTGGTTCATG |
| | ACCTTACCAAACACCCTTGCTACTAATAAAATCAAATAACACTTAGAAGGGTAT |
| | GTATTTTTAGTTAGGGTTTCTTGATCTTGGAGGATGTTTGAAAGTTAAAAATTG |
| | AATTTGGTAACCAAAGGACTGATTTATGGGTCTTTCCTATCTTAACCAACGTTTT |
| | CTTAGTTACCTAGATGGCCAAGTACAGTGCCTGGTATGTAGTAAGACTCAGTAA |
| | AAAAGTGGATTTTTAAAAATAACTCCCAAAGTGAATAGTCAAAAATCCTGTTAG |
| | CAAACTGTTATATATTGCTAAGTTTGTTCTTTTAACAGCTGGAATTTATTAAGAT |
| | GCATTATTTTGATTTTATTCACTGCCTAAAACACTTTGGGTGGTATTGATGGAGT |
| | TGGTGGATTTTCCTCCAAGTGATTAAATGAAATTTGACGTATCTTTTCATCCAAA |
| | GTTTTGTACATCATGTTTTCTAACGGAAAAAAATGTTAATATGGCTTTTTTGTAT |
| | TACTAAAAATAGCTTTGAGATTAAGGAAAAATAAATAACTCTTGTACAGTTCAG |
| | TATTGTCTATTAAATCTGTATTGGCAGTATGTATAATGGCATTTGCTGTGGTTAC |
| | AAAATACTTCCTCTGGGTTATAATAATCATTTGATCCAATTCCTATTGCTTGTAA |
| | AATAAAGTTTTACCAGTTGATATAATCAA (SEQ ID NO: 15) |
| | >NP_002347.5 myristoylated alanine-rich C-kinase |
| | substrate [Homo sapiens] |
| | MGAQFSKTAAKGEAAAERPGEAAVASSPSKANGQENGHVKVNGDASPAAAESG |
| | AKEELQANGSAPAADKEEPAAAGSGAASPSAAEKGEPAAAAAPEAGASPVEKEAP |
| | AEGEAAEPGSPTAAEGEAASAASSTSSPKAEDGATPSPSNETPKKKKKRFSFKKSFK |
| | LSGFSFKKNKKEAGEGGEAEAPAAEGGKDEAAGGAAAAAAEAGAASGEQAAAPG |
| | EEAAAGEEGAAGGDPQEAKPQEAAVAPEKPPASDETKAAEEPSKVEEKKAEEAGA |
| | SAAACEAPSAAGPGAPPEQEAAPAEEPAAAAASSACAAPSQEAQPECSPEAPPAEA |
| | AE (SEQ ID NO: 16) |
| Artificial | >Myr/Palm tag modified from Human MARCKS, nucleotide sequence |
| Sequence, | ATGGGTTGCTGTTTCTCCAAGACC (SEQ ID NO: 17) |
| modified | >Myr/Palm tag modified from Human MARCKS, peptide sequence |
| Myr/Palm tag | MGCCFSKT(SEQ ID NO: 18) |
| from Human | |
| MARCKS | |
| (underlined | |
| sequence | |
| indicates site | |
| of | |
| modification) | |
| Transmembr | >NM_001769.4 Homo sapiens CD9 molecule (CD9), transcript |
| ane domain 1 | variant 1, mRNA |
| or | AGCCGCCTGCATCTGTATCCAGCGCCAGGTCCCGCCAGTCCCAGCTGCGCGCGC |
| transmembrane | CCCCCAGTCCCGCACCCGTTCGGCCCAGGCTAAGTTAGCCCTCACCATGCCGGT |
| domain 3 | CAAAGGAGGCACCAAGTGCATCAAATACCTGCTGTTCGGATTTAACTTCATCTT |
| from Human | CTGGCTTGCCGGGATTGCTGTCCTTGCCATTGGACTATGGCTCCGATTCGACTCT |
| CD9 | CAGACCAAGAGCATCTTCGAGCAAGAAACTAATAATAATAATTCCAGCTTCTAC |
| | ACAGGAGTCTATATTCTGATCGGAGCCGGCGCCCTCATGATGCTGGTGGGCTTC |
| | CTGGGCTGCTGCGGGGCTGTGCAGGAGTCCCAGTGCATGCTGGGACTGTTCTTC |
| | GGCTTCCTCTTGGTGATATTCGCCATTGAAATAGCTGCGGCCATCTGGGGATAT |
| | TCCCACAAGGATGAGGTGATTAAGGAAGTCCAGGAGTTTTACAAGGACACCTA |
| | CAACAAGCTGAAAACCAAGGATGAGCCCCAGCGGGAAACGCTGAAAGCCATCC |
| | ACTATGCGTTGAACTGCTGTGGTTTGGCTGGGGGCGTGGAACAGTTTATCTCAG |
| | ACATCTGCCCCAAGAAGGACGTACTCGAAACCTTCACCGTGAAGTCCTGTCCTG |
| | ATGCCATCAAAGAGGTCTTCGACAATAAATTCCACATCATCGGCGCAGTGGGC |
| | ATCGGCATTGCCGTGGTCATGATATTTGGCATGATCTTCAGTATGATCTTGTGCT |
| | GTGCTATCCGCAGGAACCGCGAGATGGTCTAGAGTCAGCTTACATCCCTGAGCA |
| | GGAAAGTTTACCCATGAAGATTGGTGGGATTTTTTGTTTGTTTGTTTTGTTTTGT |
| | TTGTTGTTTGTTGTTTGTTTTTTTGCCACTAATTTTAGTATTCATTCTGCATTGCT |
| | AGATAAAAGCTGAAGTTACTTTATGTTTGTCTTTTAATGCTTCATTCAATATTGA |
| | CATTTGTAGTTGAGCGGGGGGTTTGGTTTGCTTTGGTTTATATTTTTTCAGTTGT |
| | TTGTTTTTGCTTGTTATATTAAGCAGAAATCCTGCAATGAAAGGTACTATATTTG |
| | CTAGACTCTAGACAAGATATTGTACATAAAAGAATTTTTTTGTCTTTAAATAGA |
| | TACAAATGTCTATCAACTTTAATCAAGTTGTAACTTATATTGAAGACAATTTGA |
| | TACATAATAAAAAATTATGACAATGTCCTGGA (SEQ ID NO: 19) |
| | >NP_001760.1 CD9 antigen isoform 1 [Homo sapiens] |
| | MPVKGGTKCIKYLLFGFNFIFWLAGIAVLAIGLWLRFDSQTKSIFEQETNNNNSSF |
| | YTGVYILIGAGALMMLVGFLGCCGAVQESQCMLGLFFGFLLVIFAIEIAAAIWGY |
| | SHKDEVIKEVQEFYKDTYNKLKTKDEPQRETLKAIHYALNCCGLAGGVEQFISDIC |
| | PKKDVLETFTVKSCPDAIKEVFDNKFHIIGAVGIGIA VVMIFGMIFSMILCCAIRRNR |
| | EMV (SEQ ID NO: 20) |

TABLE 2A
Abundant exosome proteins

| | | | Unique | | | | |
|---|---|---|---|---|---|---|---|
| UniProt | UniProt | Spectral | Peptide | Abundance | | | Sequence |
| Name | Accession | Count | Count | (NSAFe5) | EMPAI | Length | Coverage |
| LG3BP_HUMAN | Q08380 | 842 | 40 | 3209.7463 | 2.5399735 | 585 | 54.90% |
| MFGM_HUMAN | Q08431 | 434 | 33 | 2500.8824 | 3.7533522 | 387 | 67.70% |
| RS27A_HUMAN | P62979 | 62 | 10 | 886.3017 | 2.775722 | 156 | 57.70% |
| EDIL3_HUMAN | O43854 | 169 | 35 | 785.1632 | 2.427678 | 480 | 53.50% |
| ACTG_HUMAN | P63261 | 116 | 20 | 689.8286 | 1.6730065 | 375 | 42.70% |
| ACTB_HUMAN | P60709 | 116 | 0 | 689.8286 | 1.6730065 | 375 | 42.70% |
| ACTS_HUMAN | P68133 | 100 | 13 | 591.52506 | 0.90985334 | 377 | 28.10% |
| ACTC_HUMAN | P68032 | 100 | 0 | 591.52506 | 0.90985334 | 377 | 28.10% |
| 1433E_HUMAN | P62258 | 63 | 27 | 550.9534 | 4.1286135 | 255 | 71.00% |
| CD81_HUMAN | P60033 | 49 | 8 | 463.0187 | 1.0370419 | 236 | 30.90% |
| K2C1_HUMAN | P04264 | 128 | 50 | 443.23965 | 2.9994476 | 644 | 60.20% |
| CALM1_HUMAN | P0DP23 | 28 | 12 | 419.0697 | 2.0902956 | 149 | 49.00% |
| HS90A_HUMAN | P07900 | 135 | 48 | 411.2796 | 1.9853826 | 732 | 47.50% |
| FPRP_HUMAN | Q9P2B2 | 162 | 53 | 410.9989 | 1.9376497 | 879 | 46.80% |
| ARF3_HUMAN | P61204 | 33 | 0 | 406.5836 | 1.6607251 | 181 | 42.50% |
| ARFI_HUMAN | P84077 | 33 | 6 | 406.5836 | 1.6607251 | 181 | 42.50% |
| RAPIB_HUMAN | P61224 | 32 | 13 | 387.83469 | 1.7542286 | 184 | 44.00% |
| SDCB1_HUMAN | O00560 | 50 | 14 | 374.16937 | 2.0902956 | 298 | 49.00% |
| HS71A_HUMAN | P0DMV8 | 107 | 0 | 372.25476 | 1.9991624 | 641 | 47.70% |
| HS71B_HUMAN | P0DMV9 | 107 | 37 | 372.25476 | 1.9991624 | 641 | 47.70% |
| ATIA1_HUMAN | P05023 | 166 | 60 | 361.8653 | 1.9308932 | 1023 | 46.70% |
| ARF5_HUMAN | P84085 | 29 | 6 | 359.28574 | 1.6424088 | 180 | 42.20% |
| PDC6I_HUMAN | Q8WUM4 | 138 | 59 | 354.54703 | 2.5727282 | 868 | 55.30% |
| HS90B_HUMAN | P08238 | 114 | 42 | 351.14037 | 1.8119009 | 724 | 44.90% |
| KCRB_HUMAN | P12277 | 56 | 23 | 327.77628 | 2.7325017 | 381 | 57.20% |
| H4_HUMAN | P62805 | 15 | 11 | 324.7645 | 2.3419504 | 103 | 52.40% |
| RAPIA_HUMAN | P62834 | 26 | 13 | 315.11567 | 1.7542286 | 184 | 44.00% |
| CPNE8_HUMAN | Q86YQ8 | 78 | 38 | 308.41108 | 2.4994519 | 564 | 54.40% |
| BASP1_HUMAN | P80723 | 31 | 21 | 304.5442 | 6.4473195 | 227 | 87.20% |
| AT1B3_HUMAN | P54709 | 38 | 19 | 303.7343 | 2.0199516 | 279 | 48.00% |
| PRDX1_HUMAN | Q06830 | 27 | 15 | 302.56952 | 3.1020408 | 199 | 61.30% |
| RHOA_HUMAN | P61586 | 26 | 14 | 300.42115 | 2.2960973 | 193 | 51.80% |
| 1433Z_HUMAN | P63104 | 33 | 16 | 300.374 | 2.1768744 | 245 | 50.20% |
| PPIA_HUMAN | P62937 | 22 | 10 | 297.33994 | 2.8725765 | 165 | 58.80% |
| 1433T_HUMAN | P27348 | 31 | 15 | 282.16953 | 2.0619633 | 245 | 48.60% |
| MARCS_HUMAN | P29966 | 42 | 18 | 282.1147 | 2.6307805 | 332 | 56.00% |
| 1433F_HUMAN | Q04917 | 31 | 16 | 281.0225 | 2.810658 | 246 | 58.10% |
| GBG12_HUMAN | Q9UBI6 | 9 | 6 | 278.75616 | 4.8076444 | 72 | 76.40% |
| IF5A1_HUMAN | P63241 | 19 | 10 | 275.13597 | 2.3573759 | 154 | 52.60% |
| HSP7C_HUMAN | P11142 | 79 | 37 | 272.71502 | 1.8444612 | 646 | 45.40% |
| GNAS2_HUMAN | P63092 | 48 | 0 | 271.68114 | 1.4154608 | 394 | 38.30% |
| ARF4_HUMAN | P18085 | 21 | 4 | 260.17244 | 0.8492687 | 180 | 26.70% |
| G3P_HUMAN | P04406 | 39 | 15 | 259.6177 | 1.4099054 | 335 | 38.20% |
| K1C9_HUMAN | P35527 | 72 | 29 | 257.7264 | 3.4874535 | 623 | 65.20% |
| RHOC_HUMAN | P08134 | 22 | 10 | 254.20252 | 1.7542286 | 193 | 44.00% |
| 1433G_HUMAN | P61981 | 28 | 11 | 252.7991 | 1.42661 | 247 | 38.50% |
| ANXA2_HUMAN | P07355 | 37 | 20 | 243.39773 | 2.2809527 | 339 | 51.60% |
| CN37_HUMAN | P09543 | 45 | 27 | 238.36633 | 1.6915348 | 421 | 43.00% |
| NDKA_HUMAN | P15531 | 16 | 10 | 234.74204 | 2.2136607 | 152 | 50.70% |
| GBG10_HUMAN | P50151 | 7 | 4 | 229.56391 | 2.3806486 | 68 | 52.90% |
| AAAT_HUMAN | Q15758 | 55 | 13 | 226.71483 | 0.7458222 | 54 | 24.20% |
| 1433B_HUMAN | P31946 | 25 | 11 | 226.63104 | 1.5527012 | 246 | 40.70% |
| RAB7A_HUMAN | P51149 | 21 | 15 | 226.23688 | 4.929253 | 207 | 77.30% |
| PROFI_HUMAN | P07737 | 14 | 9 | 223.00493 | 4.0118723 | 140 | 70.00% |
| RACI_HUMAN | P63000 | 19 | 13 | 220.68198 | 2.4355795 | 192 | 53.60% |
| GNAI2_HUMAN | P04899 | 35 | 19 | 219.86403 | 1.7861211 | 355 | 44.50% |
| ATIA3_HUMAN | P13637 | 99 | 42 | 217.94166 | 1.3768401 | 1013 | 37.60% |
| COF1_HUMAN | P23528 | 16 | 11 | 214.9445 | 2.250873 | 166 | 51.20% |
| SORCN_HUMAN | P30626 | 19 | 12 | 213.99464 | 2.5563133 | 198 | 55.10% |
| ANXA6_HUMAN | P08133 | 64 | 39 | 212.07007 | 2.2359364 | 673 | 51.00% |
| PDCD6_HUMAN | O75340 | 18 | 11 | 210.16172 | 1.3227367 | 191 | 36.60% |
| KPYM_HUMAN | P14618 | 50 | 26 | 209.98581 | 2.0902956 | 531 | 49.00% |
| STMN1_HUMAN | P16949 | 14 | 6 | 209.53484 | 1.19786 | 149 | 34.20% |
| PGK1_HUMAN | P00558 | 39 | 24 | 208.56576 | 3.130475 | 417 | 61.60% |
| K1C14_HUMAN | P02533 | 44 | 32 | 207.88596 | 2.698282 | 472 | 56.80% |
| CH60_HUMAN | P10809 | 53 | 31 | 206.26984 | 1.6242187 | 573 | 41.90% |
| CLH1_HUMAN | Q00610 | 153 | 82 | 203.70004 | 1.4660394 | 1675 | 39.20% |
| EF1A1_HUMAN | P68104 | 42 | 0 | 202.73176 | 1.19786 | 462 | 34.20% |
| PEBP1_HUMAN | P30086 | 17 | 11 | 202.73176 | 2.6897762 | 187 | 56.70% |
| LSR_HUMAN | Q86X29 | 59 | 27 | 202.73176 | 1.1086283 | 649 | 32.40% |
| TBA1B_HUMAN | P68363 | 41 | 20 | 202.73176 | 2.0902956 | 451 | 49.00% |
| EF1A3_HUMAN | Q5VTE0 | 42 | 17 | 202.73176 | 1.19786 | 462 | 34.20% |
| GDIB_HUMAN | P50395 | 40 | 26 | 200.4539 | 2.2734067 | 445 | 51.50% |
| TBAIC_HUMAN | Q9BQE3 | 40 | 18 | 198.6681 | 2.1045597 | 449 | 49.20% |
| GBG5_HUMAN | P63218 | 6 | 4 | 196.76907 | 3.2854853 | 68 | 63.20% |
| GNAI3_HUMAN | P08754 | 31 | 15 | 195.28681 | 1.128139 | 354 | 32.80% |
| ANXA5_HUMAN | P08758 | 28 | 17 | 195.12932 | 1.5822601 | 320 | 41.20% |
| TPIS_HUMAN | P60174 | 25 | 15 | 194.93438 | 2.5075188 | 286 | 54.50% |
| 4F2_HUMAN | P08195 | 55 | 27 | 194.68686 | 1.5468302 | 630 | 40.60% |
| ANX11_HUMAN | P50995 | 44 | 24 | 194.30134 | 1.3878112 | 505 | 37.80% |
| K2C6B_HUMAN | P04259 | 49 | 32 | 193.74542 | 1.8119009 | 564 | 44.90% |
| CHMP5_HUMAN | Q9NZZ3 | 19 | 4 | 193.47459 | 0.91866875 | 219 | 28.30% |
| TBAIA_HUMAN | Q71U36 | 39 | 17 | 192.84241 | 1.8773985 | 451 | 45.90% |
| CD59_HUMAN | P13987 | 11 | 6 | 191.64487 | 0.7782794 | 128 | 25.00% |
| TCPQ_HUMAN | P50990 | 47 | 35 | 191.26335 | 2.9994476 | 548 | 60.20% |
| NDKB_HUMAN | P22392 | 13 | 9 | 190.72791 | 2.1117163 | 152 | 49.30% |
| SNP23_HUMAN | O00161 | 18 | 11 | 190.24119 | 3.2657952 | 211 | 63.00% |
| GBB2_HUMAN | P62879 | 29 | 12 | 190.2101 | 0.9815271 | 340 | 29.70% |
| K1C16_HUMAN | P08779 | 40 | 32 | 188.58768 | 3.1879354 | 473 | 62.20% |
| TBB5_HUMAN | P07437 | 37 | 13 | 185.83746 | 1.0230191 | 444 | 30.60% |
| RAB5C_HUMAN | P51148 | 18 | 10 | 185.83746 | 2.4833732 | 216 | 54.20% |
| ENOA_HUMAN | P06733 | 36 | 20 | 184.98106 | 2.198895 | 434 | 50.50% |
| EPCAM_HUMAN | P16422 | 26 | 11 | 184.65377 | 1.0370419 | 314 | 30.90% |
| ITB1_HUMAN | P05556 | 66 | 28 | 184.44017 | 0.8578044 | 798 | 26.90% |
| RALA_HUMAN | P11233 | 17 | 11 | 184.03319 | 1.1134889 | 206 | 32.50% |
| KIC10_HUMAN | P13645 | 48 | 27 | 183.29173 | 1.5585859 | 584 | 40.80% |
| RAP2B_HUMAN | P61225 | 15 | 9 | 182.79093 | 1.5351286 | 183 | 40.40% |
| RS8_HUMAN | P62241 | 17 | 13 | 182.26365 | 2.8194427 | 208 | 58.20% |
| IGSF8_HUMAN | Q969P0 | 50 | 27 | 181.89636 | 1.4888573 | 613 | 39.60% |
| EZRI_HUMAN | P15311 | 47 | 35 | 178.86062 | 1.5176768 | 586 | 40.10% |

TABLE 2B
Abundant exosome membrane proteins filtered by cell surface expression.

| | UniProt | UniProt | | | SURFY | Abundance |
|---|---|---|---|---|---|---|
| UniProt Name | Accession | gene | Ensembl gene | CD | Score | (NSAFe5) |
| FPRP_HUMAN | Q9P2B2 | PTGFRN | ENSG00000134247 | CD315 | 0.7864 | 410.9989 |
| ATIA1_HUMAN | P05023 | ATP1A1 | ENSG00000163399 | | 0.5464 | 361.8653 |
| AT1B3_HUMAN | P54709 | ATP1B3 | ENSG00000069849 | CD298 | 0.6797 | 303.7343 |
| AAAT_HUMAN | Q15758 | SLC1A5 | ENSG00000105281 | | 0.8104 | 226.71483 |
| AT1A3_HUMAN | P13637 | ATP1A3 | ENSG00000105409 | | 0.6786 | 217.94166 |
| 4F2_HUMAN | P08195 | SLC3A2 | ENSG00000168003 | CD98 | 0.694 | 194.68686 |
| CD59_HUMAN | P13987 | CD59 | ENSG00000085063 | CD59 | 0.7505 | 191.64487 |
| EPCAM_HUMAN | P16422 | EPCAM | ENSG00000119888 | CD326 | 0.9 | 184.65377 |
| ITB1_HUMAN | P05556 | ITGB1 | ENSG00000150093 | CD29 | 0.9588 | 184.44017 |
| IGSF8_HUMAN | Q969P0 | IGSF8 | ENSG00000162729 | CD316 | 0.486 | 181.89636 |
| ADA10_HUMAN | O14672 | ADAM10 | ENSG00000137845 | CD156c | 0.791 | 166.95557 |
| CD9_HUMAN | P21926 | CD9 | ENSG00000010278 | CD9 | 0.7385 | 166.27561 |
| AT1B1_HUMAN | P05026 | ATP1B1 | ENSG00000143153 | | 0.4759 | 161.91779 |
| CXAR_HUMAN | P78310 | CXADR | ENSG00000154639 | | 0.9535 | 152.74311 |
| ATIA2_HUMAN | P50993 | ATP1A2 | ENSG00000018625 | | 0.6507 | 148.66997 |
| BASI_HUMAN | P35613 | BSG | ENSG00000172270 | CD147 | 0.9242 | 144.8084 |
| TSN6_HUMAN | O43657 | TSPAN6 | ENSG00000000003 | | 0.7565 | 127.43139 |
| JAMI_HUMAN | Q9Y624 | F11R | ENSG00000158769 | CD321 | 0.8462 | 126.79211 |
| MOTI_HUMAN | P53985 | SLC16A1 | ENSG00000155380 | | 0.6447 | 124.88277 |
| CXA1_HUMAN | P17302 | GJA1 | ENSG00000152661 | | 0.5908 | 116.75651 |
| MPZL1_HUMAN | O95297 | MPZL1 | ENSG00000197965 | | 0.9721 | 116.06205 |
| ITA4_HUMAN | P13612 | ITGA4 | ENSG00000115232 | CD49d | 0.9162 | 112.36683 |
| EFNB2_HUMAN | P52799 | EFNB2 | ENSG00000125266 | | 0.984 | 107.14952 |
| AT2B4_HUMAN | P23634 | ATP2B4 | ENSG00000058668 | | 0.6048 | 106.02168 |
| CD151_HUMAN | P48509 | CD151 | ENSG00000177697 | CD151 | 0.7465 | 96.95867 |
| S12A2_HUMAN | P55011 | SLC12A2 | ENSG00000064651 | | 0.8343 | 95.67869 |
| CBPD_HUMAN | O75976 | CPD | ENSG00000108582 | | 0.8723 | 93.726715 |
| TSN9_HUMAN | O75954 | TSPAN9 | ENSG00000011105 | | 0.8323 | 93.307503 |
| EFNB1_HUMAN | P98172 | EFNB1 | ENSG00000090776 | | 0.9601 | 90.23321 |
| DAF_HUMAN | P08174 | CD55 | ENSG00000196352 | CD55 | 0.5988 | 87.797217 |
| ATIA4_HUMAN | Q13733 | ATP1A4 | ENSG00000132681 | | 0.4255 | 84.520824 |
| EPHA2_HUMAN | P29317 | EPHA2 | ENSG00000142627 | | 0.904 | 79.97103 |
| SATT_HUMAN | P43007 | SLC1A4 | ENSG00000115902 | | 0.8942 | 79.64462 |
| SPIT2_HUMAN | O43291 | SPINT2 | ENSG00000167642 | | 0.9521 | 79.64462 |
| EMB_HUMAN | Q6PCB8 | EMB | ENSG00000170571 | | 0.7059 | 75.01695 |
| ITA6_HUMAN | P23229 | ITGA6 | ENSG00000091409 | CD49f | 0.9405 | 74.99281 |
| S29A1_HUMAN | Q99808 | SLC29A1 | ENSG00000112759 | | 0.5476 | 73.35689 |
| CNNM3_HUMAN | Q8NE01 | CNNM3 | ENSG00000168763 | | 0.4194 | 72.54757 |
| CD276_HUMAN | Q5ZPR3 | CD276 | ENSG00000103855 | CD276 | 0.9521 | 70.99408 |
| TSN33_HUMAN | Q86UF1 | TSPAN33 | ENSG00000158457 | | 0.7345 | 70.9203 |
| MUC18_HUMAN | P43121 | MCAM | ENSG00000076706 | CD146 | 0.9613 | 69.04178 |
| PTK7_HUMAN | Q13308 | PTK7 | ENSG00000112655 | | 0.984 | 68.777223 |
| TSN14_HUMAN | Q8NG11 | TSPAN14 | ENSG00000108219 | | 0.6168 | 66.07554 |
| T106B_HUMAN | Q9NUM4 | TMEM106B | ENSG00000106460 | | 0.5868 | 65.11093 |
| JAM3_HUMAN | Q9BX67 | JAM3 | ENSG00000166086 | | 0.9579 | 64.74337 |
| CTL1_HUMAN | Q8WWI5 | SLC44A1 | ENSG00000070214 | CD92 | 0.7104 | 64.491533 |
| ITA2_HUMAN | P17301 | ITGA2 | ENSG00000164171 | CD49b | 0.9281 | 64.20126 |
| GPC4_HUMAN | O75487 | GPC4 | ENSG00000076716 | | 0.475 | 64.174086 |
| ITAV_HUMAN | P06756 | ITGAV | ENSG00000138448 | CD51 | 0.9062 | 63.83729 |
| BT2A1_HUMAN | Q7KYR7 | BTN2A1 | ENSG00000112763 | | 0.9202 | 63.473894 |

Bausch-Fluck, Damaris et al. “The in silico human surfaceome.” Proceedings of the National Academy of Sciences of the United States of America vol. 115,46 (2018): E10988-E10997.
doi: 10.1073/pnas.1808790115

In various embodiments, the multi-pass transmembrane protein is a tetraspanin or fragment thereof. In various embodiments, the at least one vesicle targeting domain further comprises a myristoylation and/or palmitoylation motif. In various embodiments, the polypeptide linker is positioned C-terminus relative to the at least one vesicle targeting domain. In various embodiments, the polypeptide linker is positioned N-terminus relative to the agonistic multi-effector domain. In various embodiments, the at least one vesicle targeting domain is a Type I transmembrane protein or fragment thereof, or a multi-pass transmembrane protein or fragment thereof. In various embodiments, the at least one vesicle targeting domain is a membrane anchoring domain from a Type I transmembrane protein or fragment thereof, or a multi-pass transmembrane protein or fragment thereof. In various embodiments, the multi-pass transmembrane protein is a tetraspanin or fragment thereof. In various embodiments, the vesicle targeting domain of the fusion polypeptide is at least one membrane anchoring domain from a multi-pass transmembrane protein wherein the multi-pass transmembrane protein is a tetraspanin or fragment thereof. In various embodiments, a fragment of a membrane anchoring domain, a fragment of a multi-pass transmembrane domain, or a fragment of a tetraspanin can be at least 75%, 80%, 85%, 90%, or 95% of a membrane anchoring domain, at least 75%, 80%, 85%, 90%, or 95% of a multi-pass transmembrane domain, or at least 75%, 80%, 85%, 90%, or 95% of a tetraspanin.

In various embodiments, the tetraspanin is CD9 (nucleic acid SEQ ID NO: 7 or amino acid SEQ ID NO: 8) or fragment thereof. In various embodiments, the fragment of CD9 is CD9tm2 (nucleic acid SEQ ID NO: 9 or amino acid SEQ ID NO: 10). In various embodiments, the membrane anchoring domain fragment of CD9 is CD9tm2. In various embodiments, the vesicle targeting domain is CD9tm2 from CD9. In various embodiments, the membrane anchoring domain has at least 85% sequence identity to CD9 SEQ ID NO: 7. In various embodiments, the CD9tm2 nucleic acid sequence has at least 85% sequence identity to SEQ ID NO: 7 or fragments thereof. In various embodiments, the CD9tm2 nucleic acid sequence has at least 85% sequence identity to SEQ ID NO: 9. In various embodiments, the fragment of CD9 is CD9tm2. In various embodiments, the CD9tm2 nucleic acid sequence has at least 90, 95, 96, 97, 98, or 99% sequence identity to SEQ ID NO: 9.

In various embodiments, the polypeptide linker is positioned N-terminus relative to the at least one vesicle targeting domain.

In various embodiments, the polypeptide linker is positioned C-terminus relative to the agonistic multi-effector domain.

In various embodiments, the at least one vesicle targeting polypeptide comprises a secretion signal recognition sequence.

In various embodiments, the secretion signal recognition sequence is positioned N-terminus relative to the agonistic multi-effector domain.

In various embodiments, the secretion signal recognition sequence is proteolytically cleaved from the agonistic multi-effector domain.

In various embodiments, a CVIM motif from KRAS-13 (CAAX box) is at the C-terminus of the fusion polypeptide.

In various embodiments, the CVIM motif is farnesylated by a post translational modification, an isoprenyl group is added to the cysteine residue, and the VIM is cleaved via proteolysis.

In various embodiments, the fusion polypeptide further comprises a linker between the agonistic multi-effector domain and the polypeptide linker.

In various embodiments, the fusion polypeptide further comprises a linker between the polypeptide linker and the at least one vesicle targeting domain.

In various embodiments, the fusion polypeptide comprises a linker between each single effector domain of the agonistic multi-effector domain.

In various embodiments, the agonistic multi-effector domain is a continuous polypeptide (i.e. single chain, sc) of three agonistic effector domains.

In various embodiments, the agonistic multi-effector domain is a continuous polypeptide (i.e. single chain, sc) of three of the same active fragments from at least one proteins of interest.

In various embodiments, each single effector domain of the agonistic multi-effector domain is a tumor necrosis factor (TNF) homology domain (THDs) or a fragment thereof.

In various embodiments, the THD is derived from a TNF superfamily member (TNFSF) selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, or TWEAK or a fragment thereof.

In various embodiments, the linker between the agonistic multi-effector domain and the polypeptide linker, the linker between the polypeptide linker and the at least one vesicle targeting domain, or the linker between single effector domains is each independently selected from the group consisting of ID, GSSG (SEQ ID NO: 154), G, GS, GGS, GGGS (SEQ ID NO: 218), GGGGS (SEQ ID NO: 156), (GGGS)n, (GGGGS) n wherein n is an integer between 1 and 10, and combinations thereof.

In various embodiments, the polypeptide linker comprises Fc or Fc mutein. In one embodiment, the Fc mutein is hIgG1 (nucleic acid sequence SEQ ID NO: 143, and amino acid sequence SEQ ID NO: 144) or a fragment thereof. In various embodiments, the Fc mutein has reduced or abolished Fc-mediated effector functions, for example, reduced or abolished Fc-Fc Receptor (FcR) mediated effector functions. In various embodiments, the FcR mutein has reduced or abolished FcγRI binding. In another embodiment, the hIgG1 is modified one or more of the following mutations L234A, L235A, and P329G (Fc-LALAPG) (nucleic acid sequence SEQ ID NO: 145, and amino acid sequence SEQ ID NO: 146; of which the L234A, L235A, P329G substitutions are indicated in bold). In various embodiments, the Fc-LALAPG mutein has reduced or abolished Fc-mediated effector functions, for example, reduced or abolished FcR or FcγRI mediated effector functions. In various embodiments, the Fc-LALAPG mutein has reduced or abolished FcγRI binding and Fc dependent cellular phagocytosis. The structural features that provide reduced or abolished FcγRI binding and Fc dependent functional effects are described in Tilman Schlothauer, Sylvia Herter, Claudia Ferrara Koller, Sandra Grau-Richards, Virginie Steinhart, Christian Spick, Manfred Kubbies, Christian Klein, Pablo Umaña, Ekkehard Mössner, Novel human IgG1 and IgG4 Fc-engineered antibodies with completely abolished immune effector functions, Protein Engineering, Design and Selection, Volume 29, Issue 10, October 2016, Pages 457-466, the contents of which is incorporated herein by reference in its entirety.

In various embodiments, the extracellular vesicle is an exosome.

Various embodiments provide for a composition comprising a plurality of the engineered extracellular vesicles of the present invention as described herein.

In various embodiments, the composition further comprising a pharmaceutically acceptable carrier.

There are various types of extracellular vesicles that are named for their site of origin in a cell, size, and structural and/or functional properties. In some embodiments of any of the aspects provided herein, the extracellular vesicle is an exosome, ectosome, macrovesicle, microparticle, apoptotic body, vesicular organelle, oncosome, exosphere, exomeres, or cell derived nanovesicle (CDN) ((e.g., by genesis via grating or shearing cells), liposomes or the like known by one of ordinary skill in the art. In various embodiments, the extracellular vesicle comprises a phospholipid bilayer with an exterior phospholipid layer and an interior phospholipid layer, wherein the exterior phospholipid layer has an external surface and an internal surface, wherein the interior phospholipid layer has an internal surface and an external surface, and the internal surface of the exterior phospholipid layer faces the internal surface of the interior phospholipid layer, and the phospholipid bilayer encloses an internal space, wherein the external surface of the interior phospholipid layer faces the internal space and wherein the external surface of the exterior phospholipid layer faces an extracellular environment, and the external surface of the inner phospholipid layer is the internal surface of the extracellular vesicle.

In various embodiments, the extracellular vesicles (EVs) range in size from 20 nanometers (nm) to 500 nm. In various embodiments, the EVs range in size from 30 nm to 300 nm. In various embodiments, the plurality of EVs range in size from about 30 nm to about 150 nm. In various embodiments, the plurality of EVs includes one or more engineered EVs that are about 10 nm to about 250 nm in diameter, including those about 10 nm to about 15 nm, about 15 nm to about 20 nm, about 20 nm to about 25 nm, about 25 nm to about 30 nm, about 30 nm to about 35 nm, about 35 nm to about 40 nm, about 40 nm to about 50 nm, about 50 nm to about 60 nm3 about 60 nm to about 70 nm, about 70 nm to about 80 nm, about 80 nm to about 90 nm, about 90 nm to about 95 nm, about 95 nm to about 100 nm, about 100 nm to about 105 nm, about 105 nm to about 110 nm, about 110 nm to about 115 nm, about 115 nm to about 120 nm, about 120 nm to about 125 nm, about 125 nm to about 130 nm, about 130 nm to about 135 nm, about 135 nm to about 140 nm, about 140 nm to about 145 nm, about 145 nm to about 150 nm, about 150 to about 200 nm, about 200 nm to about 250 nm, about 251 nm to about 300 nm, about 301 nm to about 350 nm, about 351 nm to about 400 nm, about 401 nm to about 450 nm, about 451 nm to about 500 nm, about 500 nm or more.

In some embodiments of any of the aspects provided herein, the EV is an exosome. Exosomes are plasma membrane derived vesicles that are produced in the endosomal compartment of most eukaryotic cells. As used herein, the term “exosome” refers to a species of extracellular vesicle between about 20 nm to about 500 μm in diameter, e.g., about 30 nm-200 nm in diameter. Exosomes may from by inward invagination of a portion of a membrane of an endosome (for example an early or late endosome), wherein the endosome is within a cell comprising a plasma membrane, and the exosome is released from the cell upon fusion of another portion of the endosome membrane with the plasma membrane. An exosome may refer to a species of extracellular vesicle between 20 nm-500 μm in diameter, more preferably 30 nm-200 nm in diameter, that originates by budding of a portion of a plasma membrane from a cell wherein the budded portion of the plasma membrane is released to the extracellular environment.

The EVs (e.g., exosomes or cell derived vesicles) provided herein may comprise cargo, for example, peptides, proteins, nucleic acids, lipids, metabolites, carbohydrates, biomolecules, small molecules, large molecules, vesicles, organelles, or fragments thereof. Exosome cargo may be located within the internal space of the exosome. EV cargo may be membrane bound spanning one or both layers of the exosome phospholipid bilayer (for example a transmembrane protein). EV cargo may be in contact with the exterior or interior surface of the exosome, for example through a covalent bond or a non-covalent bond. The phospholipid bilayer of the EV or exosome provided herein may comprise one or more transmembrane proteins, wherein a portion of the one or more transmembrane membrane proteins is located within the internal space of the exosome. The phospholipid bilayer of the EV or exosome provided herein may comprise one or more transmembrane proteins, wherein a portion of the one or more transmembrane membrane proteins traverses the EV phospholipid bilayer. The phospholipid bilayer of the EV may comprise one or more transmembrane proteins, wherein the one or more transmembrane membrane proteins comprises a domain on the exterior of the exosome.

In some embodiments of any of the aspects, the extracellular vesicles provided herein may display one or more endogenous biomarkers, for example CD9, CD81, CD82, CD37, CD63, CD9, CD151, CD105, ALIX, ITGB1, EpCAM, MCAM, CD133/1, CD44, NCAM, TSG101 or any combination thereof. In various embodiments, the plurality of extracellular vesicles includes one or more extracellular vesicle displaying a biomarker. In certain embodiments, the biomarkers are tetraspanins. In other embodiments, the tetraspanins are one or more selected from the group consisting of TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), TSPAN33, isoforms thereof, fragments thereof, and combinations thereof. In other embodiments, extracellular vesicles provided herein comprise one or more lipid raft associated proteins (e.g., glycosylphosphatidylinositol-anchored proteins and flotillin), cholesterol, sphingolipids such as sphingomyelin, and/or hexosylceramides. In some embodiments of any of the aspects, the extracellular vesicles provided herein comprise DAP10, DAP12, or FcεRIγ or a fragment thereof. In certain embodiments, the cytoplasmic domain of DAP10, DAP12, or FcεRIγ is deleted or does not transduce downstream cytoplasmic signaling.

In other embodiments, the biological protein is related to exosome formation and packaging of cytosolic proteins, e.g., Hsp70, Hsp90, 14-3-3 epsilon, PKM2, GW182 and AGO2. In certain embodiments, the extracellular vesicle comprises CD63, HSP70, CD105 or combinations thereof. In other embodiments, the extracellular vesicles do not display CD9 or CD81, or display neither. For example, plurality of extracellular vesicles can include one or more extracellular vesicle that are CD63+, HSP+, CD105+, CD9−, and CD81−.

The EVs provided herein are specifically engineered to display fusion polypeptides that elicit biological signaling on a target cell. In some embodiments, the fusion polypeptide is displayed on an EV to elicit a biological response on a target cell or target protein. The engineered EV comprises at least one fusion polypeptide and can comprise a plurality of the same or different fusion polypeptides provided herein. The fusion polypeptides provided herein comprise a protein of interest domain, also termed the signaling domain.

The fusion polypeptides provided herein can comprise one or more of a protein of interest or a protein of interest domain, such that expression of said fusion polypeptide is permitted and that the number of POI domains does not impede protein expression or folding. Furthermore, the EVs provided herein can display more than one fusion protein (e.g., encoded by multiple different nucleic acid constructs in a producer cell). One of skill in the art can appreciate that an engineered EV can include one or more combinations of different signaling domains and/or vesicle targeting domains, or that one can use a plurality of engineered EVs, each including one or more vesicle targeting domains and one or more signaling domains.

In some embodiments, the EVs provided herein comprise one or more, two or more, three or more, four or more, five or more, six or more, seven or more, eight or more, nine or more, or ten or more fusion polypeptides. The fusion polypeptides can be encoded by the same vector or separate vectors. In some embodiments of any of the aspects, the engineered extracellular vesicle comprises at least two POI domains and/or at least two vesicle targeting domains.

In some embodiments, the fusion polypeptide comprises one or more, two or more, three or more, four or more, five or more, or six or more POI domains on the same polypeptide or nucleic acid construct encoding said polypeptide. For example, the extracellular vesicles provided herein can comprise a fusion polypeptide encoding one or more, two or more, three or more, four or more, five or more, or six or more signaling domains. In another example, the extracellular vesicles provided herein can comprise a fusion polypeptide encoding an immune checkpoint protein or a protein involved in immune or cell synapse or any combination or fragment thereof.

In some embodiments, the EV comprises one or more, two or more, three or more, four or more, five or more, or six or more fusion polypeptides on the same EV. For example, EVs comprising one or more, two or more, three or more, four or more, five or more, or six or more fusion polypeptides wherein the fusion polypeptides encode a signaling domain. In another example, EVs comprising one or more, two or more, three or more, four or more, five or more, or six or more fusion polypeptides wherein the fusion polypeptides encode for one or more immune checkpoint proteins or proteins involved in immune or cell synapse, or any combination or fragment thereof.

### Protein of Interest

In various embodiments, the signaling domain is a protein or peptide of interest, or a fragment thereof. In various embodiments, the protein of interest (signaling domain) is an immune checkpoint protein.

The terms “signaling protein” or “signaling domain” or “protein of interest can be used interchangeably. “Protein of interest” can include a protein involved in “immune/immunological synapse” or “cell synapse” or “immune checkpoint proteins.” The immunological synapse (IS) is a stable cell-cell junction between an immune cell (e.g., a thymus-derived lymphocyte (T cell)) and at least one other cell (e.g., antigen-presenting cell (APC)). The IS refers to the organization of membrane proteins that occurs at the interface between the immune cell and at least one other cell (e.g., between the T cell and the APC) during the duration of cell-cell contact and also during the effector phase. Synapses are specialized adhesive contacts characteristic of many types of cell-cell interactions involving neurons, immune cells, epithelial cells, and even pathogens and host cells. Cell synapses play a role in cell-cell communication and coordination and is the interface where information is transmitted between adjacent cells through the exchange of signals, molecules, or physical contact between neighboring cells. Exchange of signals, molecules, or physical contact between neighboring cells may triggers a cascade of intracellular events, leading to the transmission of signals across the synapse between cells. Correct interactions between cells that form synapses require cell-cell recognition by proteins displayed on each respective cell involved in said cell-cell interactions.

In immune signaling and response, immune cell synapses involve the precise alignment of ligands and receptors (e.g., immune checkpoint receptors), leading to the activation of signaling pathways that orchestrate immune responses. Immune checkpoints receptors engage when they bind to a partner protein (e.g., ligand). Immune checkpoint protein may refer to immune checkpoint ligand or immune checkpoint receptor. In some immune checkpoint interactions, the immune checkpoint receptor and ligand partner proteins bind together, the immune checkpoint receptor may send an “off” signal to an immune cell (e.g., a T cell). In other immune checkpoint interactions, the immune checkpoint receptor and ligand partner proteins bind together, the immune checkpoint rector may send an “on” signal to an immune cell (e.g., a T cell).

The structure-function relationship of immune checkpoint receptors and ligands that provide immune checkpoint signaling are described in Rita C. Acurcio, Anna Scomparin, João Conniot, Jorge A. R. Salvador, Ronit Satchi-Fainaro, Helena F. Florindo, and Rita C. Guedes. Structure-Function Analysis of Immune Checkpoint Receptors to Guide Emerging Anticancer Immunotherapy. Journal of Medicinal Chemistry 2018 61 (24), 10957-10975. DOI: 10.1021/acs.jmedchem.8b00541 and Chin S M, Kimberlin C R, Roe-Zurz Z, Zhang P, Xu A, Liao-Chan S, Sen D, Nager A R, Oakdale N S, Brown C, Wang F, Yang Y, Lindquist K, Yeung Y A, Salek-Ardakani S, Chaparro-Riggers J. Structure of the 4-1BB/4-1BBL complex and distinct binding and functional properties of utomilumab and urelumab. Nat Commun. 2018 Nov. 8; 9 (1): 4679. doi: 10.1038/s41467-018-07136-7. PMID: 30410017; PMCID: PMC6224509, the contents of which are incorporated herein by reference in their entirety.

Signaling mechanisms and molecular interactions of immune checkpoint receptors and ligands that provide immune checkpoint signaling are described in Gaikwad S, Agrawal M Y, Kaushik I, Ramachandran S, Srivastava S K. Immune checkpoint proteins: Signaling mechanisms and molecular interactions in cancer immunotherapy. Semin Cancer Biol. 2022 November; 86 (Pt 3): 137-150. doi: 10.1016/j.semcancer.2022.03.014. Epub 2022 Mar. 24. PMID: 35341913.; and Zhang Y, Zheng J. Functions of Immune Checkpoint Molecules Beyond Immune Evasion. Adv Exp Med Biol. 2020; 1248:201-226. doi: 10.1007/978-981-15-3266-5_9. PMID: 32185712; and He, X., Xu, C. Immune checkpoint signaling and cancer immunotherapy. Cell Res 30, 660-669 (2020); and Baldanzi G. Immune Checkpoint Receptors Signaling in T Cells. Int J Mol Sci. 2022 Mar. 24; 23 (7): 3529. doi: 10.3390/ijms23073529. PMID: 35408889; PMCID: PMC8999077; and Qin S, Xu L, Yi M, Yu S, Wu K, Luo S. Novel immune checkpoint targets: moving beyond PD-1 and CTLA-4. Mol Cancer. 2019 Nov. 6; 18 (1): 155. doi: 10.1186/s12943-019-1091-2. PMID: 31690319; PMCID: PMC6833286; and Paluch C, Santos A M, Anzilotti C, Cornall R J, Davis S J. Immune Checkpoints as Therapeutic Targets in Autoimmunity. Front Immunol. 2018 Oct. 8; 9:2306. doi: 10.3389/fimmu.2018.02306. PMID: 30349540; PMCID: PMC6186808, and Mohammadi P, Hesari M, Chalabi M, Salari F, Khademi F. An overview of immune checkpoint therapy in autoimmune diseases. Int Immunopharmacol. 2022 June; 107:108647. doi: 10.1016/j.intimp.2022.108647. Epub 2022 Feb. 25. PMID: 35228033; and Zhai Y, Moosavi R, Chen M. Immune Checkpoints, a Novel Class of Therapeutic Targets for Autoimmune Diseases. Front Immunol. 2021 Apr. 21; 12:645699. doi: 10.3389/fimmu.2021.645699. PMID: 33968036; PMCID: PMC8097144, and Kucka K, Wajant H. Receptor Oligomerization and Its Relevance for Signaling by Receptors of the Tumor Necrosis Factor Receptor Superfamily. Front Cell Dev Biol. 2021 Feb. 11; 8:615141. doi: 10.3389/fcell.2020.615141. PMID: 33644033; PMCID: PMC7905041, and Croft M, Siegel R M. Beyond TNF: TNF superfamily cytokines as targets for the treatment of rheumatic diseases. Nat Rev Rheumatol. 2017 April; 13 (4): 217-233. doi: 10.1038/nrrheum.2017.22. Epub 2017 Mar. 9. PMID: 28275260; PMCID: PMC5486401, and Mayes P A, Hance K W, Hoos A. The promise and challenges of immune agonist antibody development in cancer. Nat Rev Drug Discov. 2018 July; 17 (7): 509-527. doi: 10.1038/nrd.2018.75. Epub 2018 Jun. 15. PMID: 29904196, and van der Vlist M, Kuball J, Radstake T R, Meyaard L. Immune checkpoints and rheumatic diseases: what can cancer immunotherapy teach us? Nat Rev Rheumatol. 2016 October; 12 (10): 593-604. doi: 10.1038/nrrheum.2016.131. Epub 2016 Aug. 19. PMID: 27539666, and Vanamee E S, Faustman D L. The benefits of clustering in TNF receptor superfamily signaling. Front Immunol. 2023 Aug. 17; 14:1225704. doi: 10.3389/fimmu.2023.1225704. PMID: 37662920; PMCID: PMC10469783, and Dadas O, Ertay A, Cragg M S. Delivering co-stimulatory tumor necrosis factor receptor agonism for cancer immunotherapy: past, current and future perspectives. Front Immunol. 2023 Apr. 25; 14:1147467. doi: 10.3389/fimmu.2023.1147467. PMID: 37180119; PMCID: PMC10167284, and Leonard W J, Lin J X. Strategies to therapeutically modulate cytokine action. Nat Rev Drug Discov. 2023 October; 22 (10): 827-854. doi: 10.1038/s41573-023-00746-x. Epub 2023 Aug. 4. PMID: 37542128, and Fromm G, de Silva S, Schreiber T H. Reconciling intrinsic properties of activating TNF receptors by native ligands versus synthetic agonists. Front Immunol. 2023 Sep. 19; 14:1236332. doi: 10.3389/fimmu.2023.1236332. PMID: 37795079; PMCID: PMC10546206, and Müller D. Targeting Co-Stimulatory Receptors of the TNF Superfamily for Cancer Immunotherapy. BioDrugs. 2023 January; 37 (1): 21-33. doi: 10.1007/s40259-022-00573-3. Epub 2022 Dec. 26. PMID: 36571696; PMCID: PMC9836981, and Zapata J M, Perez-Chacon G, Carr-Baena P, Martinez-Forero I, Azpilikueta A, Otano I, Melero I. CD137 (4-1BB) Signalosome: Complexity Is a Matter of TRAFs. Front Immunol. 2018 Nov. 15; 9:2618. doi: 10.3389/fimmu.2018.02618. PMID: 30524423; PMCID: PMC6262405, the contents of which are incorporated herein by reference in their entirety.

As used herein, the term “immune cell” refers to neutrophil, eosinophil, basophil, mast cell, macrophage, histocyte, Kupffer cell, alveolar macrophage, dendritic cell, B cell, plasma B cell, memory B cell, T cell, memory T cell, T helper cell, natural killer T cell, innate lymphoid cell, natural killer cell, granulocyte, monocyte, or lymphocyte, or the like as known by one of ordinary skill in the art.

Examples of “signaling protein” or “signaling domain” or “protein of interest” can include but are not limited to adenosine A2A receptor (A2AR), Galectin 9, fibrinogen-like protein 1 (FGL-1), platelet endothelial adhesion factor-1 (PECAM-1), tumor necrosis factor gene 6 protein (TSG-6), Stabilin-1 (STAB-1) also known as Clever-1, Neuropilin 1 (NRP1), Neuropilin 2 (NRP2), semaphorin-3A (SEMA3A), semaphorin-3F (SEMA3F), repulsive guidance molecule B (RGMB) also known as DRG11, T-cell immunoglobulin and mucin domain 3 (TIM-3), T cell immunoreceptor with Ig and ITIM domains (TIGIT), human leukocyte antigen (HLA) class I, HLA class II, high mobility group protein B1 (HMGB1), phosphatidylserine, carcinoembryonic antigen-related cell adhesion molecule 1 (CEACAM-1), T-cell receptor (TCR), Src homology 2 domain-containing protein tyrosine phosphatase 1 (SHP-1), SHP-2, F-Box protein 38 (FBXO38), signaling lymphocytic activation molecule (SLAM)-associated protein (SAP) also known as SH2DIA, B7RP1, indoleamine 2,3-dioxygenase (IDO), NADH oxidase 2 (NOX2), tumor necrosis factor receptor (TNFR) superfamily member 18 (TNFRSF18) (also known as activation inducible TNFR family receptor (AITR), glucocorticoid-induced TNFR related (GITR) protein, and CD357), B7-H4 also known as V-set domain containing T-cell activator inhibitor (VTCN1), B7-H5 (also known as V-domain Ig suppressor of T-cell activation (VISTA), platelet receptor Gi24, and stress induced secreted protein 1 (SISP1)), B7-H6 (also known as NCR3LG1), B7-H7 (also known as human endogenous retrovirus-H (HERV-H) long terminal repeat-associating protein 2 (HHLA2)), apelin receptor (APLNR), interferon gamma (IFN γ) receptor, programmed cell death-1 (PD-1), Protein Wnt-5a (WNT5A), serine/threonine-protein kinase PAK4, interleukin 6 (IL-6), interleukin-10 (IL-10), NKG2 family of C-type lectin receptors (for example NKG2A, B, C, D, E, F and H), ligands of NKG2 family, killer cell immunoglobulin-like receptors, CD-2, cluster of differentiation 4 (CD4), CD8, CD27, CD27 ligand (CD27L, also known as CD70), CD28, CD28H (also known as transmembrane and immunoglobulin domain containing 2 (TMIGD2) and Ig containing and proline-rich receptor-1 (IGPR1)), CD39, CD40, CD44, integrin associated protein (CD47), carcinoembryonic antigen related cell adhesion molecule 1 (CEACAM1 also known as CD66a), CD73, B7-1 (also known as CD80), B7-2 (also known as CD86), CD94, CD96, immunoglobulin superfamily member 2 (IGSF2) also known as CD101, nectin cell adhesion molecule 2 (NECTIN2) (also known as herpesvirus entry mediator B (HVEB), poliovirus receptor related 2 (PRR2, PVRL2 and PVRR2) and CD112), poliovirus receptor related immunoglobulin domain containing protein (PVIRG) also known as CD112R, CD122 (also known as IL5RB and P70-75), OX40 (also known as tumor necrosis factor receptor superfamily member 4 (TNFRSF4) and CD134), OX40 ligand (OX40L), 4-1BB (also known as CD137), CD134 (also known as 4-1BB ligand (4-1BBL) and as tumor necrosis factor ligand superfamily member 9 (TNFSF9) and CD137L), cytotoxic T-lymphocyte-associated protein 4 (CTLA-4) also known as CD152, CD154 (also known as CD40L), poliovirus receptor (PVR) also known as CD155, killer-cell immunoglobulin-like receptors (KIRs) (for example but not limited to CD158 family, CD158a, CD158g, CD158h, KIR2DL1, KIR2DS1, KIRDS3, and KIR2DS5), CD160, signal-regulatory protein alpha (SIRPa) also known as CD172a, OX-2 also known as CD200, CD200R, lymphocyte-activation gene 3 (LAG-3) also known as CD223, CD226, OX40L also known as CD252, herpes virus entry mediator (HVEM) also known as tumor necrosis factor receptor superfamily member 14 (TNFRSF14) and CD270, B- and T-lymphocyte attenuator (BTLA) also known as CD272, programmed cell death ligand-2 (PD-L2) (also known as B7-DC, PDCD1LG2, and CD273), programmed cell death-ligand 1 (PD-L1) (also known as B7-H1 and CD274), B7-H2 (also known as inducible T-cell co-stimulator ligand (ICOSLG), B7RP1, and CD275), B7-H3 also known as CD276, inducible T-cell co-stimulator (ICOS) also known as CD278, programed cell death protein 1 (PD-1) also known as CD279, leukocyte-associated Ig-like receptor-1 (LAIR-1) also known as CD305, collagen family of proteins (for example but not limited to collagen I, collagen II, collagen III alpha 1, collagen IV, collagen XXIII alpha 1, collagen XXV alpha 1), sialic acid-binding immunoglobulin-type lectin 7 (SIGLEC7) also known as CD328, sialic acid-binding immunoglobulin-type lectin 7 (SIGLEC9) also known as CD329, and natural cytotoxicity triggering receptor 3 (NKp30) also known as CD337. Isoforms or fragments thereof, or a ligand to the aforementioned proteins thereof are also included in the invention.

In some embodiments of any of the aspects, the protein of interest is a stimulatory checkpoint molecule. In certain embodiments, the stimulatory checkpoint molecule is CD27, CD28, CD40, CD122, CD137 (4-1BB), CD134 (OX40), (CD357) GITR, or CD278 (ICOS), or CD258 (LIGHT). In some embodiments of any of the aspects, the protein of interest is a ligand to a stimulatory checkpoint molecule. In certain embodiments, the ligand to the stimulatory checkpoint molecule is CD27 ligand (CD27L, also known as CD70 and TNSF7), CD80 (B7-1), CD86 (B7-2), CD40 ligand (CD40L, also known as CD154), Galectin-9, OX40 ligand (OX40L, also known as CD252), GITR ligand (GITRL), ICOS ligand (ICOSL), or HVEM.

In some embodiments of any of the aspects, the protein of interest is an inhibitory checkpoint molecule. In certain embodiments, the inhibitory checkpoint molecule is A2AR, ASBR, CD276 (B7-H3), CD272 (BTLA), CD160, CD152 (CTLA-4), IDO, TDO, CD158 family (KIR), CD223 (LAG-3), NOX2, CD279 (PD-1), TIM-3, VISTA, CD328 (SIGLEC7), or TIGIT. In certain embodiments, the ligand to the inhibitory checkpoint molecule is selected from the group consisting of netrin-1 (NET-1), HVEM, CD80 (B7-1), CD86 (B7-2), MHC-I, MHC-II, CD274 (PD-L1), CD273 (PD-L2), Galectin-9, or CD155.

In some embodiments of any of the aspects, the protein of interest is compatible with a type I membrane anchoring domain. In some embodiments of any of the aspects, the protein of interest is compatible with a type I transmembrane domain. In some embodiments of any of the aspects, the protein of interest is compatible with a vesicle targeting domain wherein the vesicle targeting domain is a type I transmembrane domain. In certain embodiments, the protein of interest that is compatible with a type I membrane anchoring domain or a type I transmembrane domain is a type I membrane protein selected from the group consisting of CD1a, CD1b, CD1c, CD1d, CD1e, LEU1 (CD5), CD6, CD7, CD10, ITGB2 (CD18), CD19, CR2 (CD21), CD27, CD28, CD34, integrin alpha-IIb (ITA2B, CD41), platelet glycoprotein IX (CD42a), platelet glycoprotein Ib alpha chain (CD42b), platelet glycoprotein Ib beta chain (CD42c), platelet glycoprotein V (CD42d), B7-1 (CD80), B7-2 (CD86), OX40 (CD134), glucocorticoid-induced TNFR-related protein (GITR, CD357), inducible T-cell costimulatory (ICOS, CD278), ICOS ligand (ICOSL, CD275), Herpes virus entry mediator A (HVEM, CD270), B7-H3 (CD276), B and T lymphocyte attenuator (BTLA, D272), CTLA-4 (CD152), killer cell immunoglobulin-like receptor family (KIR family, CD158 family: CD158a-k; KIR2DL1, KIR2DL2, KIR2DL3, KIR3DP1, KIR2DL4, KIR3DL1, KIRDs1, KIR2DL5A, KIR2D15B, KIR2DS5, KIR2DS1, KIR2DS4, KIR2DS2, KIR3DL2), PD-1 (CD279), PD-L1 (CD274), PD-L2 (CD273), T-cell immunoglobulin mucin receptor 1 (TIM-1, CD365), T-cell immunoglobulin mucin receptor 3 (TIM-3, CD366), T-cell immunoglobulin and mucin domain-containing protein 4 (TIM-4), VISTA, sialic acid-binding Ig-like lectin (SIGLEC) 1 (SIGLEC1, CD169), SIGLEC2 (CD22), SIGLEC3 (CD33), SIGLEC5 (CD170), SIGLEC6 (CD328), SIGLEC7 (CD328), SIGLEC8, SIGLEC9 (CD329), SIGLEC10, TIGIT, PVR (CD155), lysosome associated membrane glycoprotein 1 (LAMP1, CD107a), lysosome associated membrane glycoprotein 2 (LAMP2, CD107b), lysosome associated membrane glycoprotein 3 (LAMP3, CD208), PECAM-1 (CD31), STAB-1, NRP2, CEACAM-1 (CD66a), TCR, VTCN1, NCR3LG1, B7-H7 (CD28H), IFNγ receptor 1, IFNγ receptor 2, CD2, CD4, lymphocyte function-associated antigen 3 (LFA-3, CD58), CD8, CD44, CEACAM3 (CD66d), CD96, IGSF2 (CD101), NECTIN1 (HVEC, CD111), NECTIN2 (CD112), NECTIN3 (CD113), DNAX accessory molecule 1 (DNAM-1, CD226), IL2RB (CD122), tyrosine-protein phosphatase no-receptor type substate 1 (SIRPa, CD172a), signal-regulatory protein beta-1 (SIRPB1, CD172b), signal-regulatory protein gamma (SIRPG, CD172g), OX-2 (CD200), OX-2R (CD200R), LAG3 (CD223), LAIR-1 (CD305), NKp30 (CD337), TWEAKR (CD266), CD3d, CD3e, CD3g, ITGAL (CD11a), ITGAM (CD11b), ITGAX (CD11c), ITGAD (CD11d), FCGR3A (CD16a), IL-4 receptor subunit alpha (IL4RA, CD124), IL-2 receptor subunit alpha (IL2RA, CD25), ITGB1 (CD29), CD30, low affinity immunoglobulin gamma Fc region receptor II-a (CD32a), low affinity immunoglobulin gamma Fc region receptor II-b (CD32b), complement receptor type I (CD35), leukosialin (CD43), CD44, receptor-type tyrosine-protein phosphatase C (CD45), membrane cofactor protein (CD46), integrin alpha-1 (CD49a), integrin alpha-2 (CD49b), integrin alpha-3 (CD49c), integrin alpha-4 (CD49d), integrin alpha-5 (CD49e), integrin alpha-6 (CD49f), intercellular adhesion molecule 3 (ICAM-3, CD50), intercellular adhesion molecule 1 (ICAM-1, CD54), ICAM-4 (CD242), integrin alpha V (ITGAV, CD51), integrin beta 3 (ITGB3, CD61), complement decay accelerating factor (CD55), neural adhesion molecule 1 (NCAM-1, CD56), CD62E, CD62L, CD62P, High affinity immunoglobulin gamma Fc receptor I (CD64), macrosialin (CD68), B-cell antigen receptor complex-associated protein alpha chain (CD79a), B-cell antigen receptor complex-associated protein beta chain (CD79b), CD83, leukocyte immunoglobulin-like receptor subfamily A members (CD85G, CD85H, CD85I), leukocyte immunoglobulin-like receptor subfamily B members (CD85A, CD85B, CD85C, CD85D, CD85F, CD85J, CD85K), Immunoglobulin alpha Fc receptor (CD89), CD91, CD93, FAS (CD95), T-cell surface protein tactile (CD96), CD99, semaphoring-D (CD100), immunoglobulin superfamily member 2 (CD101), intercellular adhesion molecule 2 (ICAM-2, CD102), integrin alpha-E (CD103), integrin beta-4 (ITGB4, CD104), endoglin (CD105), vascular cell adhesion protein 1 (VCAM1, CD106), thrombopoietin receptor (CD110), CD114, macrophage colony-stimulating factor 1 receptor (CSF1R, CD115), Granulocyte-macrophage colony-stimulating factor receptor subunit alpha (CSF2RA, CD116), mast/stem cell growth factor receptor Kit (CD117), leukemia inhibitory factor receptor (LIFR, CD118), interferon gamma receptor 1 (CD119), Tumor necrosis factor receptor superfamily member 1A (TNF-R1, CD120a), Tumor necrosis factor receptor superfamily member 1B (TNF-R2, CD120b), Interleukin-1 receptor type 1 (CD121a), Interleukin-1 receptor type 2 (CD121b), Interleukin-2 receptor subunit beta (CD122), Interleukin-3 receptor subunit alpha (IL3RA, CD123), Interleukin-4 receptor subunit alpha (IL4RA, CD124), Interleukin-5 receptor subunit alpha (IL5RA, CD125), Interleukin-6 receptor subunit alpha (IL6RA, CD126), Interleukin-6 receptor subunit beta (IL6ST, CD130), Interleukin-7 receptor subunit alpha (IL7RA, CD127), Interleukin-9 receptor (CD129), Cytokine receptor common subunit beta (CD131), Cytokine receptor common subunit gamma (CD132), CD135, macrophage stimulating protein receptor (CD136), syndecan-1 (CD138), Platelet-derived growth factor receptor alpha (PDGFRA, CD140a), Platelet-derived growth factor receptor beta (PDGFRB, CD140b), thrombomodulin (CD141), CD142, angiotensin converting enzyme (ACE, CD143), cadherin-5 (CD144), melanoma and adhesion molecule (MCAM, CD146), basigin (BSG, CD147), CD148, Signaling lymphocytic activation molecule (SLAM, CD150), SLAM family member 4 (SLAMF4, CD244), signaling lymphocytic activation molecule (SLAM) family member 5 (SLAM5, CD84), SLAM family member 6 (SLAMF6, CD352), SLAM family member 7 (SLAMF7, CD319), SLAM family member 8 (SLAMF8, CD353), SLAM family member 9 (SLAM9), Disintegrin and metalloproteinase domain-containing protein 8 (ADAM8, CD156a), Disintegrin and metalloproteinase domain-containing protein 17 (ADAM17, CD156b), Disintegrin and metalloproteinase domain-containing protein 10 (ADAM10, CD156c), P-selectin glycoprotein 1 (SELPLG, CD162), CD163, CD164, activated leukocyte cell adhesion molecule (ALCAM, CD166), epithelial discoidin domain containing receptor 1 (CD167a), discoidin domain containing receptor 2 (CD167b), neural cell adhesion molecule L1 (L1CAM, CD 171), CD180, endothelial protein C receptor (EPCR, CD201), angiopoietin-1 receptor (CD202b), lymphocyte antigen 75 (CD205), macrophage mannose receptor 1 (CD206), IL-10 receptor subunit alpha (IL10RA, CD210), IL-10 receptor subunit beta (IL10RB, CDw210b), IL-12 receptor subunit beta-1 (IL12RB1, CD212), IL-13 receptor subunit alpha-1 (CD213a1), IL-13 receptor subunit alpha-2 (CD213a2), IL-15 receptor subunit alpha (CD215), IL-17 receptor A (CD217), IL-18 receptor 1 (CD218a), IL-18 receptor accessory protein (CD218b), insulin receptor (CD220), insulin-like growth factor 1 receptor (CD221), cation-independent mannos-6phosphate receptor (CD222), mucin-1 (CD227), T-lymphocyte surface antigen Ly-9 (CD229), plexin-C1 (VESPR, CD232), glycophorin-A (CD235a), glycophorin-B (CD235b), basal cell adhesion molecule (CD239), CD246, T-cell surface glycoprotein CD3 zeta chain (CD247), endosialin (CD248), death receptor 3 (DR3, TNFRS25), death receptor 4 (DR4, CD261), death receptor 5 (DR5, CD262), decoy receptor 2 (DcR2, CD264), receptor activator of nuclear factor kappa-B (RANK, CD265), CD271, C-type mannose receptor 2 (CD280), Toll like receptor 1 (CD281), Toll like receptor 2 (CD282), Toll like receptor 3 (CD283), Toll like receptor 4 (CD284), Toll like receptor 6 (CD286), Toll like receptor 8 (CD288), Toll like receptor 9 (CD289), Toll like receptor 10 (CD290), bone morphogenic protein receptor type 1A (CD292), bone morphogenic protein receptor type ID (CwD293), leptin receptor (CD295), CD300a, CD300c, CD302, Neuropilin-1 (CD304), leukocyte-associated immunoglobulin-like receptor 1 (LIAR1, CD305), Fc receptor-like protein 1 (FcRL1, CD307a), Fc receptor-like protein 2 (FcRL2, CD307b), Fc receptor-like protein 3 (FcRL3, CD307c), Fc receptor-like protein 4 (FcRL4, CD307d), Fc receptor-like protein 5 (FcRL5, CD307e), vascular endothelial growth factor receptor 2 (VEGFR2, CD309), prostaglandin F2 receptor negative regulator (PTGFRN, CD315), immunoglobulin superfamily member 8 (IGSF8, CD316), CD320, platelet F11 receptor (F11R, CD321), junctional adhesion molecule B (JAM-B, CD322), cadherin-1 (CD324), cadherin-2 (CD325), epithelial cell adhesion molecule (CD326), fibroblast growth factor 1 (FGFR1, CD331), fibroblast growth factor 2 (FGFR2, CD332), fibroblast growth factor 3 (FGFR3, CD333), fibroblast growth factor 4 (FGFR4, CD334), natural cytotoxicity triggering receptor 1 (NCR1, CD335), natural cytotoxicity triggering receptor 2 (NCR2, CD336), natural cytotoxicity triggering receptor 3 (NCR3, CD337), triggering receptor expressing on myeloid cells 1 (TREM1, CD354), cytotoxic and regulatory T-cell molecule (CRTAM, CD355), tumor necrosis factor receptor superfamily member 21 (CD358), interleukin-21 receptor (IL21R, CD360), protein EVI2B (CD361), syndecan-2 (CD362), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), butyrophilin subfamily 3 member A1 (BTN3A1, CD277), butyrophilin subfamily 3 member A2 (BTN3A2), butyrophilin subfamily 2 member A1 (BTN2A1), butyrophilin like protein 8 (BTNL8), butyrophilin subfamily 1 member A1 (BTN1A1), lymphotoxin beta receptor (LTBR), isoforms thereof fragments thereof, a ligand to the aforementioned proteins thereof, a receptor to the aforementioned proteins thereof, and combinations thereof.

In some embodiments of any of the aspects, the protein of interest that is compatible with a type I membrane anchoring domain is a GPI anchored protein. In certain embodiments, the GPI anchored protein is selected from the group consisting of CD160, RGMB, CEACAM8 (CD66b, CD67), CEACAM6 (CD66c), CEACAM5 (CD66e), CD73, CD14, FCGR3B (CD16b), CD24, BLAST-1 (CD48), CAMPATH-1 (CD52), CD59, CD87, CD90, semaphorin-7A (CD108), CD109, bone marrow stromal cell antigen 1 (BST1, CD157), CD177, melanotransferrin (CD228), CD230, decoy receptor 1 (DcR1, CD263), CD296, CD297 isoforms thereof, fragments thereof, a ligand to the aforementioned proteins thereof, a receptor to the aforementioned proteins thereof, and combinations thereof.

In some embodiments of any of the aspects, the protein of interest is compatible with a type I membrane anchoring domain. In some embodiments of any of the aspects, the protein of interest that is compatible with a type I membrane anchoring domain is a type III membrane protein. In certain embodiments, the type III membrane protein is selected from the group consisting of B cell activating factor (BAFFR, CD268), glycophorin-C(CD236), transmembrane activator and CAML interactor (TACI, CD267), B-cell maturation protein (BCM, CD269), isoforms thereof, fragments thereof, a ligand to the aforementioned proteins thereof, a receptor to the aforementioned proteins thereof, and combinations thereof.

In some embodiments of any of the aspects, the protein of interest that is compatible with a type I membrane anchoring domain is a secreted protein. In certain embodiments, the type I membrane anchoring domain compatible secreted protein is selected from the group consisting of Interleukin (IL) 1 (IL1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-34, IL-35, IL-36, IL-37, IL-38, IL-39, IL-40, fibrinogen-like protein 1 (FGL-1), TSG-6, NRP1, SEMA3A, SEMA3F, IFNγ, WNT5A, PSG1 (CD66f), collagen family of proteins (for example but not limited to collagen I, collagen II, collagen III alpha 1, collagen IV, collagen XXIII alpha 1, collagen XXV alpha 1), A proliferation-inducing ligand (APRIL, CD256), pregnancy-specific beta-1-glycoprotein 1 (PSG1, CD66f), leukocyte-associated immunoglobulin-like receptor 2 (LIAR2, CD306), peptidase inhibitor 16 (CD364), netrin-1 (NET-1), colony-stimulating factor (CSF), decoy receptor 3 (DcR3, TNFRSF6B), isoforms thereof, fragments thereof, a ligand to the aforementioned proteins thereof, a receptor to the aforementioned proteins thereof, and combinations thereof.

In some embodiments of any of the aspects, the protein of interest is compatible with a type II membrane anchoring domain. In some embodiments of any of the aspects, the protein of interest is compatible with a type II transmembrane domain. In some embodiments of any of the aspects, the protein of interest is compatible with a vesicle targeting domain wherein the vesicle targeting domain is a type II transmembrane domain. In certain embodiments, the protein of interest that is compatible with a type II membrane anchoring domain or a type II transmembrane domain is a type II membrane protein selected from the group consisting of CD27L (CD70), CD40, CD40L (CD154), 4-1BB (CD137), 4-1BBL (CD137L), OX40L (CD252), glucocorticoid-induced TNF-related ligand (GITRL), LIGHT (CD258), TNF-related apoptosis inducing factor (TRAIL, CD253), CLEC7A (CD369), CD30L (CD153), TL1 (TNFSF15), FasL (CD178), NKG2 family ligands (NKG2A, B, C, D, E, F and H), B cell activating factor (BAFF, CD257), TNF-related weak inducer of apoptosis (TWEAK), RBAT (SLC3A1), ATP1B2, CD94, neprilysin (CD10), CD13, BLAST-2 (CD23), Dipeptidyl peptidase 4 (DPP4, ADCP2, CD26), CD38, CLEC2C (CD69), Transferrin receptor protein 1 (CD71), B-cell differentiation antigen Lyb-2 (CD72), HLA class II histocompatibility antigen gamma chain (CD74), CD75, CD77, natural killer cell antigen KLRD1 (CD94), NKG2-A/B-activating NK receptor (CD159a), NKG2-C(CD159c), killer cell lectin-like receptor subfamily B member 1 (CD161), galactoside alpha-(1,2)-fucosyltransferase 1 (FUT1, CD174), 3-galactosyl-N-acetylglucosamide 4-alpha-L-fucosyltransferase (FUT3), ectonucleotide pyrophosphatase/phosphodiesterase (ENPP) family member 1 (ENPP1, CD203a), ENPP family member 3 (ENPP3, CD203c), macrophage scavenger receptor types I and II (CD204), C-type lectin domain family 4 member K (langerin, CD207), dendritic cell-specific ICAM-3-grabbing non-integrin 1 (DC-SIGN, CD209), CD224, CD238, glutamyl aminopeptidase (CD249), receptor activator of nuclear factor kappa-B ligand (RANKL, CD254), CD298, DC-SIGN related protein (DC-SIGNR, CD299), C-type lectin domain family 10 member A (CLEC10A, CD301), C-type lectin domain family 4 member C (CLEC4C, CD303), NKG2-D type II integral membrane protein (KLRK1, CD314), bone marrow stromal antigen 2 (BST2, CD317), transmembrane and associated with src kinases (TRASK, CD318), protein jagged-1 (CD339), human epidermal growth factor 2 (HER2, CD340), C-type lectin domain family 4 member A (CLEC4A, CD367), C-type lectin domain family 4 member D (CLEC4D, CD368), C-type lectin domain family 7 member A (CLEC7A, CD369), C-type lectin domain family 9 member A (CLEC9A, CD370), C-type lectin domain family 12 member A (CLEC12A, CD371), SLC3A2 (CD98 heavy chain), tumor necrosis factor (TNF, TNF-alpha, TNFα), lymphotoxin-alpha (LTA, LT-α) also known as tumor necrosis factor ligand superfamily member 1 (TNF-beta, TNF-β), tumor necrosis family ligand superfamily member 3 also known as lymphotoxin beta (LTB, TNF-C, TNFγ), tumor necrosis factor ligand superfamily 15 (TL1A), A proliferation-inducing ligand (APRIL, CD256), isoforms thereof, fragments thereof, a ligand to the aforementioned proteins thereof, a receptor to the aforementioned proteins thereof, and combinations thereof.

In some embodiments of any of the aspects, the protein of interest that is compatible with a type II membrane anchoring domain is a secreted protein. In certain embodiments, the secreted protein is selected from the group consisting of Interleukin (IL) 1 (IL1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-34, IL-35, IL-36, IL-37, IL-38, IL-39, IL-40, fibrinogen-like protein 1 (FGL-1), TSG-6, NRP1, SEMA3A, SEMA3F, IFNγ, WNT5A, PSG1 (CD66f), collagen family of proteins (for example but not limited to collagen I, collagen II, collagen III alpha 1, collagen IV, collagen XXIII alpha 1, collagen XXV alpha 1), A proliferation-inducing ligand (APRIL, CD256), pregnancy-specific beta-1-glycoprotein 1 (PSG1, CD66f), leukocyte-associated immunoglobulin-like receptor 2 (LIAR2, CD306), peptidase inhibitor 16 (CD364), netrin-1 (NET-1), colony-stimulating factor (CSF), decoy receptor 3 (DcR3, TNFRSF6B), isoforms thereof, fragments thereof, a ligand to the aforementioned proteins thereof, a receptor to the aforementioned proteins thereof, and combinations thereof.

In some embodiments of any of the aspects provided herein, the protein of interest domain (POI domain) comprises a polypeptide or a fragment thereof or a nucleic acid encoding said polypeptide or fragment thereof from Table 3A and 3B (below). Non-limiting examples of nucleic acid sequences that encode the POI domains provided herein are also provided in Table 3A and 3B.

TABLE 3A
Proteins of Interest compatible with a Type I membrane
anchoring domain (i.e. a type I vesicle targeting domain)

| Protein of | Transcript Sequence (SEQ ID NO:) |
|---|---|
| Interest | Amino Acid Sequence (SEQ ID NO:) |
| Human | >NM_014143.4 Homo sapiens CD274 molecule (CD274), |
| Programmed | transcript variant 1, mRNA, nucleic acid sequence |
| death-ligand | AGTTCTGCGCAGCTTCCCGAGGCTCCGCACCAGCCGCGCTTCTGTCCGCCTGCA |
| 1 (PD-L1) | GGGCATTCCAGAAAGATGAGGATATTTGCTGTCTTTATATTCATGACCTACTGG |
| | CATTTGCTGAACGCATTTACTGTCACGGTTCCCAAGGACCTATATGTGGTAGAG |
| | TATGGTAGCAATATGACAATTGAATGCAAATTCCCAGTAGAAAAACAATTAGA |
| | CCTGGCTGCACTAATTGTCTATTGGGAAATGGAGGATAAGAACATTATTCAATT |
| | TGTGCATGGAGAGGAAGACCTGAAGGTTCAGCATAGTAGCTACAGACAGAGGG |
| | CCCGGCTGTTGAAGGACCAGCTCTCCCTGGGAAATGCTGCACTTCAGATCACAG |
| | ATGTGAAATTGCAGGATGCAGGGGTGTACCGCTGCATGATCAGCTATGGTGGTG |
| | CCGACTACAAGCGAATTACTGTGAAAGTCAATGCCCCATACAACAAAATCAAC |
| | CAAAGAATTTTGGTTGTGGATCCAGTCACCTCTGAACATGAACTGACATGTCAG |
| | GCTGAGGGCTACCCCAAGGCCGAAGTCATCTGGACAAGCAGTGACCATCAAGT |
| | CCTGAGTGGTAAGACCACCACCACCAATTCCAAGAGAGAGGAGAAGCTTTTCA |
| | ATGTGACCAGCACACTGAGAATCAACACAACAACTAATGAGATTTTCTACTGCA |
| | CTTTTAGGAGATTAGATCCTGAGGAAAACCATACAGCTGAATTGGTCATCCCAG |
| | AACTACCTCTGGCACATCCTCCAAATGAAAGGACTCACTTGGTAATTCTGGGAG |
| | CCATCTTATTATGCCTTGGTGTAGCACTGACATTCATCTTCCGTTTAAGAAAAGG |
| | GAGAATGATGGATGTGAAAAAATGTGGCATCCAAGATACAAACTCAAAGAAGC |
| | AAAGTGATACACATTTGGAGGAGACGTAATCCAGCATTGGAACTTCTGATCTTC |
| | AAGCAGGGATTCTCAACCTGTGGTTTAGGGGTTCATCGGGGCTGAGCGTGACAA |
| | GAGGAAGGAATGGGCCCGTGGGATGCAGGCAATGTGGGACTTAAAAGGCCCAA |
| | GCACTGAAAATGGAACCTGGCGAAAGCAGAGGAGGAGAATGAAGAAAGATGG |
| | AGTCAAACAGGGAGCCTGGAGGGAGACCTTGATACTTTCAAATGCCTGAGGGG |
| | CTCATCGACGCCTGTGACAGGGAGAAAGGATACTTCTGAACAAGGAGCCTCCA |
| | AGCAAATCATCCATTGCTCATCCTAGGAAGACGGGTTGAGAATCCCTAATTTGA |
| | GGGTCAGTTCCTGCAGAAGTGCCCTTTGCCTCCACTCAATGCCTCAATTTGTTTT |
| | CTGCATGACTGAGAGTCTCAGTGTTGGAACGGGACAGTATTTATGTATGAGTTT |
| | TTCCTATTTATTTTGAGTCTGTGAGGTCTTCTTGTCATGTGAGTGTGGTTGTGAA |
| | TGATTTCTTTTGAAGATATATTGTAGTAGATGTTACAATTTTGTCGCCAAACTAA |
| | ACTTGCTGCTTAATGATTTGCTCACATCTAGTAAAACATGGAGTATTTGTAAGG |
| | TGCTTGGTCTCCTCTATAACTACAAGTATACATTGGAAGCATAAAGATCAAACC |
| | GTTGGTTGCATAGGATGTCACCTTTATTTAACCCATTAATACTCTGGTTGACCTA |
| | ATCTTATTCTCAGACCTCAAGTGTCTGTGCAGTATCTGTTCCATTTAAATATCAG |
| | CTTTACAATTATGTGGTAGCCTACACACATAATCTCATTTCATCGCTGTAACCAC |
| | CCTGTTGTGATAACCACTATTATTTTACCCATCGTACAGCTGAGGAAGCAAACA |
| | GATTAAGTAACTTGCCCAAACCAGTAAATAGCAGACCTCAGACTGCCACCCACT |
| | GTCCTTTTATAATACAATTTACAGCTATATTTTACTTTAAGCAATTCTTTTATTCA |
| | AAAACCATTTATTAAGTGCCCTTGCAATATCAATCGCTGTGCCAGGCATTGAAT |
| | CTACAGATGTGAGCAAGACAAAGTACCTGTCCTCAAGGAGCTCATAGTATAAT |
| | GAGGAGATTAACAAGAAAATGTATTATTACAATTTAGTCCAGTGTCATAGCATA |
| | AGGATGATGCGAGGGGAAAACCCGAGCAGTGTTGCCAAGAGGAGGAAATAGG |
| | CCAATGTGGTCTGGGACGGTTGGATATACTTAAACATCTTAATAATCAGAGTAA |
| | TTTTCATTTACAAAGAGAGGTCGGTACTTAAAATAACCCTGAAAAATAACACTG |
| | GAATTCCTTTTCTAGCATTATATTTATTCCTGATTTGCCTTTGCCATATAATCTAA |
| | TGCTTGTTTATATAGTGTCTGGTATTGTTTAACAGTTCTGTCTTTTCTATTTAAAT |
| | GCCACTAAATTTTAAATTCATACCTTTCCATGATTCAAAATTCAAAAGATCCCAT |
| | GGGAGATGGTTGGAAAATCTCCACTTCATCCTCCAAGCCATTCAAGTTTCCTTTC |
| | CAGAAGCAACTGCTACTGCCTTTCATTCATATGTTCTTCTAAAGATAGTCTACAT |
| | TTGGAAATGTATGTTAAAAGCACGTATTTTTAAAATTTTTTTCCTAAATAGTAAC |
| | ACATTGTATGTCTGCTGTGTACTTTGCTATTTTTATTTATTTTAGTGTTTCTTATA |
| | TAGCAGATGGAATGAATTTGAAGTTCCCAGGGCTGAGGATCCATGCCTTCTTTG |
| | TTTCTAAGTTATCTTTCCCATAGCTTTTCATTATCTTTCATATGATCCAGTATATG |
| | TTAAATATGTCCTACATATACATTTAGACAACCACCATTTGTTAAGTATTTGCTC |
| | TAGGACAGAGTTTGGATTTGTTTATGTTTGCTCAAAAGGAGACCCATGGGCTCT |
| | CCAGGGTGCACTGAGTCAATCTAGTCCTAAAAAGCAATCTTATTATTAACTCTG |
| | TATGACAGAATCATGTCTGGAACTTTTGTTTTCTGCTTTCTGTCAAGTATAAACT |
| | TCACTTTGATGCTGTACTTGCAAAATCACATTTTCTTTCTGGAAATTCCGGCAGT |
| | GTACCTTGACTGCTAGCTACCCTGTGCCAGAAAAGCCTCATTCGTTGTGCTTGA |
| | ACCCTTGAATGCCACCAGCTGTCATCACTACACAGCCCTCCTAAGAGGCTTCCT |
| | GGAGGTTTCGAGATTCAGATGCCCTGGGAGATCCCAGAGTTTCCTTTCCCTCTT |
| | GGCCATATTCTGGTGTCAATGACAAGGAGTACCTTGGCTTTGCCACATGTCAAG |
| | GCTGAAGAAACAGTGTCTCCAACAGAGCTCCTTGTGTTATCTGTTTGTACATGT |
| | GCATTTGTACAGTAATTGGTGTGACAGTGTTCTTTGTGTGAATTACAGGCAAGA |
| | ATTGTGGCTGAGCAAGGCACATAGTCTACTCAGTCTATTCCTAAGTCCTAACTC |
| | CTCCTTGTGGTGTTGGATTTGTAAGGCACTTTATCCCTTTTGTCTCATGTTTCATC |
| | GTAAATGGCATAGGCAGAGATGATACCTAATTCTGCATTTGATTGTCACTTTTT |
| | GTACCTGCATTAATTTAATAAAATATTCTTATTTATTTTGTTACTTGGTACACCA |
| | GCATGTCCATTTTCTTGTTTATTTTGTGTTTAATAAAATGTTCAGTTTAACATCCC |
| | A (SEQ ID NO: 21) |
| | >NP_054862.1 programmed cell death 1 ligand 1 isoform a |
| | precursor [Homo sapiens], amino acid sequence |
| | MRIFAVFIFMTYWHLLNAFTVTVPKDLYVVEYGSNMTIECKFPVEKQLDLAALIVY |
| | WEMEDKNIIQFVHGEEDLKVQHSSYRQRARLLKDQLSLGNAALQITDVKLQDAGV |
| | YRCMISYGGADYKRITVKVNAPYNKINQRILVVDPVTSEHELTCQAEGYPKAEVIW |
| | TSSDHQVLSGKTTTTNSKREEKLFNVTSTLRINTTTNEIFYCTFRRLDPEENHTAELV |
| | IPELPLAHPPNERTHLVILGAILLCLGVALTFIFRLRKGRMMDVKKCGIQDTNSKKQ |
| | SDTHLEET (SEQ ID NO: 22) |
| Human | >NM_025239.4 Homo sapiens programmed cell death 1 ligand |
| PD-L2 | 2 (PDCD1LG2), mRNA, nucleic acid sequence |
| | ACTCTCATGTTACGGCAAACCTTAAGCTGAATGAACAACTTTTCTTCTCTTGAAT |
| | ATATCTTAACGCCAAATTTTGAGTGCTTTTTTGTTACCCATCCTCATATGTCCCA |
| | GCTAGAAAGAATCCTGGGTTGGAGCTACTGCATGTTGATTGTTTTGTTTTTCCTT |
| | TTGGCTGTTCATTTTGGTGGCTACTATAAGGAAATCTAACACAAACAGCAACTG |
| | TTTTTTGTTGTTTACTTTTGCATCTTTACTTGTGGAGCTGTGGCAAGTCCTCATAT |
| | CAAATACAGAACATGATCTTCCTCCTGCTAATGTTGAGCCTGGAATTGCAGCTT |
| | CACCAGATAGCAGCTTTATTCACAGTGACAGTCCCTAAGGAACTGTACATAATA |
| | GAGCATGGCAGCAATGTGACCCTGGAATGCAACTTTGACACTGGAAGTCATGT |
| | GAACCTTGGAGCAATAACAGCCAGTTTGCAAAAGGTGGAAAATGATACATCCC |
| | CACACCGTGAAAGAGCCACTTTGCTGGAGGAGCAGCTGCCCCTAGGGAAGGCC |
| | TCGTTCCACATACCTCAAGTCCAAGTGAGGGACGAAGGACAGTACCAATGCAT |
| | AATCATCTATGGGGTCGCCTGGGACTACAAGTACCTGACTCTGAAAGTCAAAGC |
| | TTCCTACAGGAAAATAAACACTCACATCCTAAAGGTTCCAGAAACAGATGAGG |
| | TAGAGCTCACCTGCCAGGCTACAGGTTATCCTCTGGCAGAAGTATCCTGGCCAA |
| | ACGTCAGCGTTCCTGCCAACACCAGCCACTCCAGGACCCCTGAAGGCCTCTACC |
| | AGGTCACCAGTGTTCTGCGCCTAAAGCCACCCCCTGGCAGAAACTTCAGCTGTG |
| | TGTTCTGGAATACTCACGTGAGGGAACTTACTTTGGCCAGCATTGACCTTCAAA |
| | GTCAGATGGAACCCAGGACCCATCCAACTTGGCTGCTTCACATTTTCATCCCCT |
| | TCTGCATCATTGCTTTCATTTTCATAGCCACAGTGATAGCCCTAAGAAAACAAC |
| | TCTGTCAAAAGCTGTATTCTTCAAAAGACACAACAAAAAGACCTGTCACCACAA |
| | CAAAGAGGGAAGTGAACAGTGCTATCTGAACCTGTGGTCTTGGGAGCCAGGGT |
| | GACCTGATATGACATCTAAAGAAGCTTCTGGACTCTGAACAAGAATTCGGTGGC |
| | CTGCAGAGCTTGCCATTTGCACTTTTCAAATGCCTTTGGATGACCCAGCACTTTA |
| | ATCTGAAACCTGCAACAAGACTAGCCAACACCTGGCCATGAAACTTGCCCCTTC |
| | ACTGATCTGGACTCACCTCTGGAGCCTATGGCTTTAAGCAAGCACTACTGCACT |
| | TTACAGAATTACCCCACTGGATCCTGGACCCACAGAATTCCTTCAGGATCCTTC |
| | TTGCTGCCAGACTGAAAGCAAAAGGAATTATTTCCCCTCAAGTTTTCTAAGTGA |
| | TTTCCAAAAGCAGAGGTGTGTGGAAATTTCCAGTAACAGAAACAGATGGGTTG |
| | CCAATAGAGTTATTTTTTATCTATAGCTTCCTCTGGGTACTAGAAGAGGCTATTG |
| | AGACTATGAGCTCACAGACAGGGCTTCGCACAAACTCAAATCATAATTGACAT |
| | GTTTTATGGATTACTGGAATCTTGATAGCATAATGAAGTTGTTCTAATTAACAG |
| | AGAGCATTTAAATATACACTAAGTGCACAAATTGTGGAGTAAAGTCATCAAGCT |
| | CTGTTTTTGAGGTCTAAGTCACAAAGCATTTGTTTTAACCTGTAATGGCACCATG |
| | TTTAATGGTGGTTTTTTTTTTGAACTACATCTTTCCTTTAAAAATTATTGGTTTCT |
| | TTTTATTTGTTTTTACCTTAGAAATCAATTATATACAGTCAAAAATATTTGATAT |
| | GCTCATACGTTGTATCTGCAGCAATTTCAGATAAGTAGCTAAAATGGCCAAAGC |
| | CCCAAACTAAGCCTCCTTTTCTGGCCCTCAATATGACTTTAAATTTGACTTTTCA |
| | GTGCCTCAGTTTGCACATCTGTAATACAGCAATGCTAAGTAGTCAAGGCCTTTG |
| | ATAATTGGCACTATGGAAATCCTGCAAGATCCCACTACATATGTGTGGAGCAGA |
| | AGGGTAACTCGGCTACAGTAACAGCTTAATTTTGTTAAATTTGTTCTTTATACTG |
| | GAGCCATGAAGCTCAGAGCATTAGCTGACCCTTGAACTATTCAAATGGGCACAT |
| | TAGCTAGTATAACAGACTTACATAGGTGGGCCTAAAGCAAGCTCCTTAACTGAG |
| | CAAAATTTGGGGCTTATGAGAATGAAAGGGTGTGAAATTGACTAACAGACAAA |
| | TCATACATCTCAGTTTCTCAATTCTCATGTAAATCAGAGAATGCCTTTAAAGAAT |
| | AAAACTCAATTGTTATTCTTCAACGTTCTTTATATATTCTACTTTTGGGTA |
| | (SEQ ID NO: 23) |
| | >NP_079515.2 programmed cell death 1 ligand 2 precursor |
| | [Homo sapiens], amino acid sequence |
| | MIFLLLMLSLELQLHQIAALFTVTVPKELYIIEHGSNVTLECNFDTGSHVNLGAITAS |
| | LQKVENDTSPHRERATLLEEQLPLGKASFHIPQVQVRDEGQYQCIIIYGVAWDYKY |
| | LTLKVKASYRKINTHILKVPETDEVELTCQATGYPLAEVSWPNVSVPANTSHSRTPE |
| | GLYQVTSVLRLKPPPGRNFSCVFWNTHVRELTLASIDLQSQMEPRTHPTWLLHIFIP |
| | FCIIAFIFIATVIALRKQLCQKLYSSKDTTKRPVTTTKREVNSAI (SEQ ID NO: 24) |
| Human | >NM_005214.5 Homo sapiens cytotoxic T-lymphocyte |
| CTLA-4 | associated protein 4 (CTLA4), transcript variant 1, |
| (CD152) | mRNA, nucleic acid sequence |
| | GCTTTCTATTCAAGTGCCTTCTGTGTGTGCACATGTGTAATACATATCTGGGATC |
| | AAAGCTATCTATATAAAGTCCTTGATTCTGTGTGGGTTCAAACACATTTCAAAG |
| | CTTCAGGATCCTGAAAGGTTTTGCTCTACTTCCTGAAGACCTGAACACCGCTCC |
| | CATAAAGCCATGGCTTGCCTTGGATTTCAGCGGCACAAGGCTCAGCTGAACCTG |
| | GCTACCAGGACCTGGCCCTGCACTCTCCTGTTTTTTCTTCTCTTCATCCCTGTCTT |
| | CTGCAAAGCAATGCACGTGGCCCAGCCTGCTGTGGTACTGGCCAGCAGCCGAG |
| | GCATCGCCAGCTTTGTGTGTGAGTATGCATCTCCAGGCAAAGCCACTGAGGTCC |
| | GGGTGACAGTGCTTCGGCAGGCTGACAGCCAGGTGACTGAAGTCTGTGCGGCA |
| | ACCTACATGATGGGGAATGAGTTGACCTTCCTAGATGATTCCATCTGCACGGGC |
| | ACCTCCAGTGGAAATCAAGTGAACCTCACTATCCAAGGACTGAGGGCCATGGA |
| | CACGGGACTCTACATCTGCAAGGTGGAGCTCATGTACCCACCGCCATACTACCT |
| | GGGCATAGGCAACGGAACCCAGATTTATGTAATTGATCCAGAACCGTGCCCAG |
| | ATTCTGACTTCCTCCTCTGGATCCTTGCAGCAGTTAGTTCGGGGTTGTTTTTTTA |
| | TAGCTTTCTCCTCACAGCTGTTTCTTTGAGCAAAATGCTAAAGAAAAGAAGCCC |
| | TCTTACAACAGGGGTCTATGTGAAAATGCCCCCAACAGAGCCAGAATGTGAAA |
| | AGCAATTTCAGCCTTATTTTATTCCCATCAATTGAGAAACCATTATGAAGAAGA |
| | GAGTCCATATTTCAATTTCCAAGAGCTGAGGCAATTCTAACTTTTTTGCTATCCA |
| | GCTATTTTTATTTGTTTGTGCATTTGGGGGGAATTCATCTCTCTTTAATATAAAG |
| | TTGGATGCGGAACCCAAATTACGTGTACTACAATTTAAAGCAAAGGAGTAGAA |
| | AGACAGAGCTGGGATGTTTCTGTCACATCAGCTCCACTTTCAGTGAAAGCATCA |
| | CTTGGGATTAATATGGGGATGCAGCATTATGATGTGGGTCAAGGAATTAAGTTA |
| | GGGAATGGCACAGCCCAAAGAAGGAAAAGGCAGGGAGCGAGGGAGAAGACTA |
| | TATTGTACACACCTTATATTTACGTATGAGACGTTTATAGCCGAAATGATCTTTT |
| | CAAGTTAAATTTTATGCCTTTTATTTCTTAAACAAATGTATGATTACATCAAGGC |
| | TTCAAAAATACTCACATGGCTATGTTTTAGCCAGTGATGCTAAAGGTTGTATTG |
| | CATATATACATATATATATATATATATATATATATATATATATATATATATATAT |
| | ATATATATATTTTAATTTGATAGTATTGTGCATAGAGCCACGTATGTTTTTGTGT |
| | ATTTGTTAATGGTTTGAATATAAACACTATATGGCAGTGTCTTTCCACCTTGGGT |
| | CCCAGGGAAGTTTTGTGGAGGAGCTCAGGACACTAATACACCAGGTAGAACAC |
| | AAGGTCATTTGCTAACTAGCTTGGAAACTGGATGAGGTCATAGCAGTGCTTGAT |
| | TGCGTGGAATTGTGCTGAGTTGGTGTTGACATGTGCTTTGGGGCTTTTACACCA |
| | GTTCCTTTCAATGGTTTGCAAGGAAGCCACAGCTGGTGGTATCTGAGTTGACTT |
| | GACAGAACACTGTCTTGAAGACAATGGCTTACTCCAGGAGACCCACAGGTATG |
| | ACCTTCTAGGAAGCTCCAGTTCGATGGGCCCAATTCTTACAAACATGTGGTTAA |
| | TGCCATGGACAGAAGAAGGCAGCAGGTGGCAGAATGGGGTGCATGAAGGTTTC |
| | TGAAAATTAACACTGCTTGTGTTTTTAACTCAATATTTTCCATGAAAATGCAACA |
| | ACATGTATAATATTTTTAATTAAATAAAAATCTGTGGTGGTCGTTTTCCGGA |
| | (SEQ ID NO: 25) |
| | >NP_005205.2 cytotoxic T-lymphocyte protein 4 isoform |
| | CTLA4-TM precursor [Homo sapiens], amino acid sequence |
| | MACLGFQRHKAQLNLATRTWPCTLLFFLLFIPVFCKAMHVAQPAVVLASSRGIASF |
| | VCEYASPGKATEVRVTVLRQADSQVTEVCAATYMMGNELTFLDDSICTGTSSGNQ |
| | VNLTIQGLRAMDTGLYICKVELMYPPPYYLGIGNGTQIYVIDPEPCPDSDFLLWILA |
| | AVSSGLFFYSFLLTAVSLSKMLKKRSPLTTGVYVKMPPTEPECEKQFQPYFIPIN |
| | (SEQ ID NO: 26) |
| Human | >NM_003820.4 Homo sapiens TNF receptor superfamily |
| HVEM | member 14 (TNFRSF14), transcript variant 1, mRNA, |
| (CD270) | nucleic acid sequence |
| | ATACCGGCCCTTCCCCTCGGCTTTGCCTGGACAGCTCCTGCCTCCCGCAGGGCC |
| | CACCTGTGTCCCCCAGCGCCGCTCCACCCAGCAGGCCTGAGCCCCTCTCTGCTG |
| | CCAGACACCCCCTGCTGCCCACTCTCCTGCTGCTCGGGTTCTGAGGCACAGCTT |
| | GTCACACCGAGGCGGATTCTCTTTCTCTTTCTCTTTCTCTTCTGGCCCACAGCCG |
| | CAGCAATGGCGCTGAGTTCCTCTGCTGGAGTTCATCCTGCTAGCTGGGTTCCCG |
| | AGCTGCCGGTCTGAGCCTGAGGCATGGAGCCTCCTGGAGACTGGGGGCCTCCTC |
| | CCTGGAGATCCACCCCCAAAACCGACGTCTTGAGGCTGGTGCTGTATCTCACCT |
| | TCCTGGGAGCCCCCTGCTACGCCCCAGCTCTGCCGTCCTGCAAGGAGGACGAGT |
| | ACCCAGTGGGCTCCGAGTGCTGCCCCAAGTGCAGTCCAGGTTATCGTGTGAAGG |
| | AGGCCTGCGGGGAGCTGACGGGCACAGTGTGTGAACCCTGCCCTCCAGGCACC |
| | TACATTGCCCACCTCAATGGCCTAAGCAAGTGTCTGCAGTGCCAAATGTGTGAC |
| | CCAGCCATGGGCCTGCGCGCGAGCCGGAACTGCTCCAGGACAGAGAACGCCGT |
| | GTGTGGCTGCAGCCCAGGCCACTTCTGCATCGTCCAGGACGGGGACCACTGCGC |
| | CGCGTGCCGCGCTTACGCCACCTCCAGCCCGGGCCAGAGGGTGCAGAAGGGAG |
| | GCACCGAGAGTCAGGACACCCTGTGTCAGAACTGCCCCCCGGGGACCTTCTCTC |
| | CCAATGGGACCCTGGAGGAATGTCAGCACCAGACCAAGTGCAGCTGGCTGGTG |
| | ACGAAGGCCGGAGCTGGGACCAGCAGCTCCCACTGGGTATGGTGGTTTCTCTCA |
| | GGGAGCCTCGTCATCGTCATTGTTTGCTCCACAGTTGGCCTAATCATATGTGTGA |
| | AAAGAAGAAAGCCAAGGGGTGATGTAGTCAAGGTGATCGTCTCCGTCCAGCGG |
| | AAAAGACAGGAGGCAGAAGGTGAGGCCACAGTCATTGAGGCCCTGCAGGCCCC |
| | TCCGGACGTCACCACGGTGGCCGTGGAGGAGACAATACCCTCATTCACGGGGA |
| | GGAGCCCAAACCACTGACCCACAGACTCTGCACCCCGACGCCAGAGATACCTG |
| | GAGCGACGGCTGCTGAAAGAGGCTGTCCACCTGGCGGAACCACCGGAGCCCGG |
| | AGGCTTGGGGGCTCCGCCCTGGGCTGGCTTCCGTCTCCTCCAGTGGAGGGAGAG |
| | GTGGGGCCCCTGCTGGGGTAGAGCTGGGGACGCCACGTGCCATTCCCATGGGC |
| | CAGTGAGGGCCTGGGGCCTCTGTTCTGCTGTGGCCTGAGCTCCCCAGAGTCCTG |
| | AGGAGGAGCGCCAGTTGCCCCTCGCTCACAGACCACACACCCAGCCCTCCTGG |
| | GCCAGCCCAGAGGGCCCTTCAGACCCCAGCTGTCTGCGCGTCTGACTCTTGTGG |
| | CCTCAGCAGGACAGGCCCCGGGCACTGCCTCACAGCCAAGGCTGGACTGGGTT |
| | GGCTGCAGTGTGGTGTTTAGTGGATACCACATCGGAAGTGATTTTCTAAATTGG |
| | ATTTGAATTCGGCTCCTGTTTTCTATTTGTCATGAAACAGTGTATTTGGGGAGAT |
| | GCTGTGGGAGGATGTAAATATCTTGTTTCTCCTCAAA (SEQ ID NO: 27) |
| | >NP_003811.2 tumor necrosis factor receptor superfamily |
| | member 14 isoform 1 precursor [Homo sapiens], amino acid |
| | sequence |
| | MEPPGDWGPPPWRSTPKTDVLRLVLYLTFLGAPCYAPALPSCKEDEYPVGSECCPK |
| | CSPGYRVKEACGELTGTVCEPCPPGTYIAHLNGLSKCLQCQMCDPAMGLRASRNC |
| | SRTENAVCGCSPGHFCIVQDGDHCAACRAYATSSPGQRVQKGGTESQDTLCQNCP |
| | PGTFSPNGTLEECQHQTKCSWLVTKAGAGTSSSHWVWWFLSGSLVIVIVCSTVGLII |
| | CVKRRKPRGDVVKVIVSVQRKRQEAEGEATVIEALQAPPDVTTVAVEETIPSFTGRS |
| | PNH (SEQ ID NO: 28) |
| Human | >NM_004467.4 Homo sapiens fibrinogen like 1 (FGL1), |
| FGL1 | transcript variant 1, mRNA, nucleic acid sequence |
| | AATGCAGTTACAGGATCCTGGGAAGCAGAGTGTCTGGATGGAACCTGAGCTGG |
| | GTCTCTGACTCACTTCTGACTTTAGTTTTTTCAAGGGGGAACATGGCAAAGGTG |
| | TTCAGTTTCATCCTTGTTACCACCGCTCTGACAATGGGCAGGGAAATTTCGGCG |
| | CTCGAGGACTGTGCCCAGGAGCAGATGCGGCTCAGAGCCCAGGTGCGCCTGCT |
| | TGAGACCCGGGTCAAACAGCAACAGGTCAAGATCAAGCAGCTTTTGCAGGAGA |
| | ATGAAGTCCAGTTCCTTGATAAAGGAGATGAGAATACTGTCATTGATCTTGGAA |
| | GCAAGAGGCAGTATGCAGATTGTTCAGAGATTTTCAATGATGGGTATAAGCTCA |
| | GTGGATTTTACAAAATCAAACCTCTCCAGAGCCCAGCAGAATTTTCTGTTTATT |
| | GTGACATGTCCGATGGAGGAGGATGGACTGTAATTCAGAGACGATCTGATGGC |
| | AGTGAAAACTTTAACAGAGGATGGAAAGACTATGAAAATGGCTTTGGAAATTT |
| | TGTCCAAAAACATGGTGAATATTGGCTGGGCAATAAAAATCTTCACTTCTTGAC |
| | CACTCAAGAAGACTACACTTTAAAAATCGACCTTGCAGATTTTGAAAAAAATAG |
| | CCGTTATGCACAATATAAGAATTTCAAAGTTGGAGATGAAAAGAATTTCTACGA |
| | GTTGAATATTGGGGAATATTCTGGAACAGCTGGAGATTCCCTTGCGGGGAATTT |
| | TCATCCTGAGGTGCAGTGGTGGGCTAGTCACCAAAGAATGAAATTCAGCACGT |
| | GGGACAGAGATCATGACAACTATGAAGGGAACTGCGCAGAAGAAGATCAGTCT |
| | GGCTGGTGGTTTAACAGGTGTCACTCTGCAAACCTGAATGGTGTATACTACAGC |
| | GGCCCCTACACGGCTAAAACAGACAATGGGATTGTCTGGTACACCTGGCATGG |
| | GTGGTGGTATTCTCTGAAATCTGTGGTTATGAAAATTAGGCCAAATGATTTTATT |
| | CCAAATGTAATTTAATTGCTGCTGTTGGGCTTTCGTTTCTGCAATTCAGCTTTGT |
| | TTAAAGTGATTTGAAAAATACTCATTCTGAACATATCCATGCGCAATCATGATA |
| | ACTGTTGTGAGTAGTGCTTTTCATTCTTCTCACTTGCCTTTGTTACTTAATGTGCT |
| | TTCAGTACAGCAGATATGCAATATTCACCAAATAAATGTAGACTGTGTTAATA |
| | (SEQ ID NO: 29) |
| | >NP_004458.3 fibrinogen-like protein 1 precursor |
| | [Homo sapiens], amino acid sequence |
| | MAKVFSFILVTTALTMGREISALEDCAQEQMRLRAQVRLLETRVKQQQVKIKQLL |
| | QENEVQFLDKGDENTVIDLGSKRQYADCSEIFNDGYKLSGFYKIKPLQSPAEFSVYC |
| | DMSDGGGWTVIQRRSDGSENFNRGWKDYENGFGNFVQKHGEYWLGNKNLHFLT |
| | TQEDYTLKIDLADFEKNSRYAQYKNFKVGDEKNFYELNIGEYSGTAGDSLAGNFHP |
| | EVQWWASHQRMKFSTWDRDHDNYEGNCAEEDQSGWWFNRCHSANLNGVYYSG |
| | PYTAKTDNGIVWYTWHGWWYSLKSVVMKIRPNDFIPNVI (SEQ ID NO: 30) |
| Human OX-2 | >NM_005944.7 Homo sapiens CD200 molecule (CD200), |
| (CD200) | transcript variant 1, mRNA, nucleic acid sequence |
| | AGAGCTCCAGGCGCACATCCGCAGTCAGCCACCTCGCGCGCGCCTCCAGGAGC |
| | AAGGATGGAGAGGCTGGTGATCAGGATGCCCTTCTCTCATCTGTCTACCTACAG |
| | CCTGGTTTGGGTCATGGCAGCAGTGGTGCTGTGCACAGCACAAGTGCAAGTGGT |
| | GACCCAGGATGAAAGAGAGCAGCTGTACACACCTGCTTCCTTAAAATGCTCTCT |
| | GCAAAATGCCCAGGAAGCCCTCATTGTGACATGGCAGAAAAAGAAAGCTGTAA |
| | GCCCAGAAAACATGGTCACCTTCAGCGAGAACCATGGGGTGGTGATCCAGCCT |
| | GCCTATAAGGACAAGATAAACATTACCCAGCTGGGACTCCAAAACTCAACCAT |
| | CACCTTCTGGAATATCACCCTGGAGGATGAAGGGTGTTACATGTGTCTCTTCAA |
| | TACCTTTGGTTTTGGGAAGATCTCAGGAACGGCCTGCCTCACCGTCTATGTACA |
| | GCCCATAGTATCCCTTCACTACAAATTCTCTGAAGACCACCTAAATATCACTTG |
| | CTCTGCCACTGCCCGCCCAGCCCCCATGGTCTTCTGGAAGGTCCCTCGGTCAGG |
| | GATTGAAAATAGTACAGTGACTCTGTCTCACCCAAATGGGACCACGTCTGTTAC |
| | CAGCATCCTCCATATCAAAGACCCTAAGAATCAGGTGGGGAAGGAGGTGATCT |
| | GCCAGGTGCTGCACCTGGGGACTGTGACCGACTTTAAGCAAACCGTCAACAAA |
| | GGCTATTGGTTTTCAGTTCCGCTATTGCTAAGCATTGTTTCCCTGGTAATTCTTC |
| | TCGTCCTAATCTCAATCTTACTGTACTGGAAACGTCACCGGAATCAGGACCGAG |
| | AGCCCTAAATAAGTCACACAGCACCCTGAAAGTGATTCCCTGGTCTACTTGAAT |
| | TTGACACAAGAGAAAAGCAGGAGGAAAAGGGGCCATTCTCCAAAGGACCTGA |
| | AAGAGCAAAAGAGGTGGGAGCGAAAGCCTTAAGGATCCCACGACTTTTTACTG |
| | CCATCTGAGCTACTCAGTGTTTGAATCCCAAGAGGAAGTCAGTTTACCTCTCAG |
| | GTCTGTTGTAGGACTTGATTTTGTAAAGCAATGCCATGTTATGTGGTTGAAAGG |
| | GCACTGGACTTAGTTAGTATCAGGAGCACTGAGCTCACAGACTGACTTGGGCTC |
| | CTACTGGTGGGGACCTCTGTTAGTCACTTTACCTCATCCAAAGTATAAAGGAAT |
| | TGGACCAAATAATTTACCACATAGCTCTAAAACTTAATTTAAAATGTAATTCCA |
| | GAAAAAAAAAGGGAATAAGCAAAGGGGGAAGAATTGAAAGAGAGAGAGAAG |
| | AAAGAATACAGAGAGCTTACCTTTTGCCTTTCTGTTGATGTTACATCTCTTCTTC |
| | CTATGTTCTTAGGTCTATGAGTCTGTTTCCCCATCATTTGGTATCTAGTCCAGTT |
| | CCTGCTTACTGCTTTGCTAATAGCTGGCCTTGCTAGAATCCTTGGTTTCACTGCT |
| | GTTCTTCATGTGCTTCTATGAGATTTACTCCAACACAAATAGGACTGAATTTATT |
| | GTGAAGTAACATTGGCAATCTTAACTTATTCATTTAACTTATTTTTATAGCTAGA |
| | TAAATATTGTTAGTCTTAGACAATAGCTCACATTTTTTGAGAAGCATGCCCTCCC |
| | TGTCCATTTGTCTTATAACATGACCCAGCCCTATTTTACGTCATTCTAAATTCAG |
| | CCTCATATAATGAAAATACATTATGAAAACAGATGTTTAGGAGATTTCCTGTAT |
| | AGCAGTCAGCCAATTCATATGCTTTGTCTCTGCTGGCTTCTTTTTCCATGCGTTA |
| | ACTTTTCCCAATAGCAGAGGAGGCAAATATGAGCATACAATCCCTTTGTTCTAA |
| | AGATATTGTTCCAGCTAGTGGAATGATGTTGAATCTTTAATAACCATAATTAGT |
| | TGCTTTTTCAGTATCTTCTGCTTTGTCTGTGTCTATCCAGTGGCCTAGGAATTAA |
| | AGTGTAAGTTGTTTTCGCTGTTAAATTGGATATTTATATATATATATAGCAAGAT |
| | TTTCATGTGTTATTTAATTCTGTATTGTTTCTTATATTTGTAGTAAAATATTGAAC |
| | AATTAAAAGTGTTGACTCCAAA (SEQ ID NO: 31) |
| | >NP_005935.4 OX-2 membrane glycoprotein isoform a |
| | precursor [Homo sapiens], amino acid sequence |
| | MERLVIRMPFSHLSTYSLVWVMAAVVLCTAQVQVVTQDEREQLYTPASLKCSLQN |
| | AQEALIVTWQKKKAVSPENMVTFSENHGVVIQPAYKDKINITQLGLQNSTITFWNIT |
| | LEDEGCYMCLFNTFGFGKISGTACLTVYVQPIVSLHYKFSEDHLNITCSATARPAPM |
| | VFWKVPRSGIENSTVTLSHPNGTTSVTSILHIKDPKNQVGKEVICQVLHLGTVTDFK |
| | QTVNKGYWFSVPLLLSIVSLVILLVLISILLYWKRHRNQDREP (SEQ ID NO: 32) |
| Human | >NM_009587.3 Homo sapiens galectin 9 (LGALS9), |
| Galectin-9 | transcript variant 1, mRNA, nucleic acid sequence |
| | CTTTGTTAAGTCGTTCCCTCTACAAAGGACTTCCTAGTGGGTGTGAAAGGCAGC |
| | GGTGGCCACAGAGGCGGCGGAGAGATGGCCTTCAGCGGTTCCCAGGCTCCCTA |
| | CCTGAGTCCAGCTGTCCCCTTTTCTGGGACTATTCAAGGAGGTCTCCAGGACGG |
| | ACTTCAGATCACTGTCAATGGGACCGTTCTCAGCTCCAGTGGAACCAGGTTTGC |
| | TGTGAACTTTCAGACTGGCTTCAGTGGAAATGACATTGCCTTCCACTTCAACCCT |
| | CGGTTTGAAGATGGAGGGTACGTGGTGTGCAACACGAGGCAGAACGGAAGCTG |
| | GGGGCCCGAGGAGAGGAAGACACACATGCCTTTCCAGAAGGGGATGCCCTTTG |
| | ACCTCTGCTTCCTGGTGCAGAGCTCAGATTTCAAGGTGATGGTGAACGGGATCC |
| | TCTTCGTGCAGTACTTCCACCGCGTGCCCTTCCACCGTGTGGACACCATCTCCGT |
| | CAATGGCTCTGTGCAGCTGTCCTACATCAGCTTCCAGAACCCCCGCACAGTCCC |
| | TGTTCAGCCTGCCTTCTCCACGGTGCCGTTCTCCCAGCCTGTCTGTTTCCCACCC |
| | AGGCCCAGGGGGCGCAGACAAAAACCTCCCGGCGTGTGGCCTGCCAACCCGGC |
| | TCCCATTACCCAGACAGTCATCCACACAGTGCAGAGCGCCCCTGGACAGATGTT |
| | CTCTACTCCCGCCATCCCACCTATGATGTACCCCCACCCCGCCTATCCGATGCCT |
| | TTCATCACCACCATTCTGGGAGGGCTGTACCCATCCAAGTCCATCCTCCTGTCA |
| | GGCACTGTCCTGCCCAGTGCTCAGAGGTTCCACATCAACCTGTGCTCTGGGAAC |
| | CACATCGCCTTCCACCTGAACCCCCGTTTTGATGAGAATGCTGTGGTCCGCAAC |
| | ACCCAGATCGACAACTCCTGGGGGTCTGAGGAGCGAAGTCTGCCCCGAAAAAT |
| | GCCCTTCGTCCGTGGCCAGAGCTTCTCAGTGTGGATCTTGTGTGAAGCTCACTG |
| | CCTCAAGGTGGCCGTGGATGGTCAGCACCTGTTTGAATACTACCATCGCCTGAG |
| | GAACCTGCCCACCATCAACAGACTGGAAGTGGGGGGCGACATCCAGCTGACCC |
| | ATGTGCAGACATAGGCGGCTTCCTGGCCCTGGGGCCGGGGGCTGGGGTGTGGG |
| | GCAGTCTGGGTCCTCTCATCATCCCCACTTCCCAGGCCCAGCCTTTCCAACCCTG |
| | CCTGGGATCTGGGCTTTAATGCAGAGGCCATGTCCTTGTCTGGTCCTGCTTCTGG |
| | CTACAGCCACCCTGGAACGGAGAAGGCAGCTGACGGGGATTGCCTTCCTCAGC |
| | CGCAGCAGCACCTGGGGCTCCAGCTGCTGGAATCCTACCATCCCAGGAGGCAG |
| | GCACAGCCAGGGAGAGGGGAGGAGTGGGCAGTGAAGATGAAGCCCCATGCTC |
| | AGTCCCCTCCCATCCCCCACGCAGCTCCACCCCAGTCCCAAGCCACCAGCTGTC |
| | TGCTCCTGGTGGGAGGTGGCCTCCTCAGCCCCTCCTCTCTGACCTTTAACCTCAC |
| | TCTCACCTTGCACCGTGCACCAACCCTTCACCCCTCCTGGAAAGCAGGCCTGAT |
| | GGCTTCCCACTGGCCTCCACCACCTGACCAGAGTGTTCTCTTCAGAGGACTGGC |
| | TCCTTTCCCAGTGTCCTTAAAATAAAGAAATGAAAATGCTTGTTGGCACATTCA |
| | (SEQ ID NO: 33) |
| | >NP_033665.1 galectin-9 isoform long [Homo sapiens], |
| | amino acid sequence |
| | MAFSGSQAPYLSPAVPFSGTIQGGLQDGLQITVNGTVLSSSGTRFAVNFQTGFSGND |
| | IAFHFNPRFEDGGYVVCNTRQNGSWGPEERKTHMPFQKGMPFDLCFLVQSSDFKV |
| | MVNGILFVQYFHRVPFHRVDTISVNGSVQLSYISFQNPRTVPVQPAFSTVPFSQPVCF |
| | PPRPRGRRQKPPGVWPANPAPITQTVIHTVQSAPGQMFSTPAIPPMMYPHPAYPMPF |
| | ITTILGGLYPSKSILLSGTVLPSAQRFHINLCSGNHIAFHLNPRFDENAVVRNTQIDNS |
| | WGSEERSLPRKMPFVRGQSFSVWILCEAHCLKVAVDGQHLFEYYHRLRNLPTINRL |
| | EVGGDIQLTHVQT (SEQ ID NO: 34) |
| Human PVR | >NM_006505.5 Homo sapiens PVR cell adhesion molecule (PVR), |
| (CD155) | transcript variant 1, mRNA, nucleic acid sequence |
| | AGTCACTTGTCTGGAGCTTGAAGAAGTGGGTATTCCCCTTCCCACCCCAGGCAC |
| | TGGAGGAGCGGCCCCCCGGGGATTCCAGGACCTGAGCTCCGGGAGCTGGACTC |
| | GCAGCGACCGCGGCAGAGCGAGCGGGCGCCGGGAAGCGAGGAGACGCCCGCG |
| | GGAGGCCCAGCTGCTCGGAGCAACTGGCATGGCCCGAGCCATGGCCGCCGCGT |
| | GGCCGCTGCTGCTGGTGGCGCTACTGGTGCTGTCCTGGCCACCCCCAGGAACCG |
| | GGGACGTCGTCGTGCAGGCGCCCACCCAGGTGCCCGGCTTCTTGGGCGACTCCG |
| | TGACGCTGCCCTGCTACCTACAGGTGCCCAACATGGAGGTGACGCATGTGTCAC |
| | AGCTGACTTGGGCGCGGCATGGTGAATCTGGCAGCATGGCCGTCTTCCACCAAA |
| | CGCAGGGCCCCAGCTATTCGGAGTCCAAACGGCTGGAATTCGTGGCAGCCAGA |
| | CTGGGCGCGGAGCTGCGGAATGCCTCGCTGAGGATGTTCGGGTTGCGCGTAGA |
| | GGATGAAGGCAACTACACCTGCCTGTTCGTCACGTTCCCGCAGGGCAGCAGGA |
| | GCGTGGATATCTGGCTCCGAGTGCTTGCCAAGCCCCAGAACACAGCTGAGGTTC |
| | AGAAGGTCCAGCTCACTGGAGAGCCAGTGCCCATGGCCCGCTGCGTCTCCACA |
| | GGGGGTCGCCCGCCAGCCCAAATCACCTGGCACTCAGACCTGGGCGGGATGCC |
| | CAATACGAGCCAGGTGCCAGGGTTCCTGTCTGGCACAGTCACTGTCACCAGCCT |
| | CTGGATATTGGTGCCCTCAAGCCAGGTGGACGGCAAGAATGTGACCTGCAAGG |
| | TGGAGCACGAGAGCTTTGAGAAGCCTCAGCTGCTGACTGTGAACCTCACCGTGT |
| | ACTACCCCCCAGAGGTATCCATCTCTGGCTATGATAACAACTGGTACCTTGGCC |
| | AGAATGAGGCCACCCTGACCTGCGATGCTCGCAGCAACCCAGAGCCCACAGGC |
| | TATAATTGGAGCACGACCATGGGTCCCCTGCCACCCTTTGCTGTGGCCCAGGGC |
| | GCCCAGCTCCTGATCCGTCCTGTGGACAAACCAATCAACACAACTTTAATCTGC |
| | AACGTCACCAATGCCCTAGGAGCTCGCCAGGCAGAACTGACCGTCCAGGTCAA |
| | AGAGGGACCTCCCAGTGAGCACTCAGGCATGTCCCGTAACGCCATCATCTTCCT |
| | GGTTCTGGGAATCCTGGTTTTTCTGATCCTGCTGGGGATCGGGATTTATTTCTAT |
| | TGGTCCAAATGTTCCCGTGAGGTCCTTTGGCACTGTCATCTGTGTCCCTCGAGTA |
| | CAGAGCATGCCAGCGCCTCAGCTAATGGGCATGTCTCCTATTCAGCTGTGAGCA |
| | GAGAGAACAGCTCTTCCCAGGATCCACAGACAGAGGGCACAAGGTGACAGCGT |
| | CGGGACTGAGAGGGGAGAGAGACTGGAGCTGGCAAGGACGTGGGCCTCCAGA |
| | GTTGGACCCGACCCCAATGGATGAAGACCCCCTCCAAAGAGACCAGCCTCCCTC |
| | CCTGTGCCAGACCTCAAAACGACGGGGGCAGGTGCAAGTTCATAGGTCTCCAA |
| | GACCACCCTCCTTTCATTTGCTAGAAGGACTCACTAGACTCAGGAAAGCTGTTA |
| | GGCTCACAGTTACAGTTTATTACAGTAAAAGGACAGAGATTAAGATCAGCAAA |
| | GGGAGGAGGTGCACAGCACACGTTCCACGACAGATGAGGCGACGGCTTCCATC |
| | TGCCCTCTCCCAGTGGAGCCATATAGGCAGCACCTGATTCTCACAGCAACATGT |
| | GACAACATGCAAGAAGTACTGCCAATACTGCCAACCAGAGCAGCTCACTCGAG |
| | ATCTTTGTGTCCAGAGTTTTTTGTTTGTCTTGAGACAGGGTCTGGCTCTGTTGGC |
| | AGACTAGAGTACAGTGGTGAGATCACAGTTCATTGCAGCCTTGACTTCTCAACG |
| | CCAAGTCATCCTCCCACCTCAGCCTCCTGAGTAGCTATGACTACAGGTATGTGC |
| | CACCACGTCTGGCTAATCTTTTTATTATTTGTAAAGTCGAGGTTTCCCTGTGTTG |
| | CCCAGGCTGGTCTTGAACTCTTGGCTCCAAGTGATACTTCTGCCTTGGCCTCCCA |
| | AAGTGCTGAATTAAGCAGCTCACCATCCACACGGCTGACCTCATACATCAAGCC |
| | AATACCGTGTGGCCCAAGACCCCCACCATAAATCACATCATTAGCATGAACCAC |
| | CCAGAGTGGCCCAAGACTCCAAGATCAGCTACCAGGCAGGATATTCCAAGGGC |
| | TTAGAGATGAATGCCCAGGAGCTGAGGATAAAGGGCCCGATCTTTCTTTGGGCA |
| | AGGTTAAGCCTTTACTGCATAGCAGACCACACAGAAGGGTGTGGGCCACCAGA |
| | GAATTTTGGTAAAAATTTGGCCTCTGGCCTTGAGCTTCTAAATCTCTGTATCCGT |
| | CAGATCTCTGTGGTTACAAGAAACAGCCACTGACCCTGGTCACCAGAGGCTGCA |
| | ATTCAGGCCGCAAGCAGCTGCCTGGGGGGTGTCCAAGGAGCAGAGAAAACTAC |
| | TAGATGTGAACTTGAAGAAGGTTGTCAGCTGCAGCCACTTTCTGCCAGCATCTG |
| | CAGCCACTTTCTGCCAGCATCTGCAGCCAGCAAGCTGGGACTGGCAGGAAATA |
| | ACCCACAAAAGAAGCAAATGCAATTTCCAACACAAGGGGGAAGGGATGCAGG |
| | GGGAGGCAGCGCTGCAGTTGCTCAGGACACGCTCCTATAGGACCAAGATGGAT |
| | GCGACCCAAGACCCAGGAGGCCCAGCTGCTCAGTGCAACTGACAAGTTAAAAA |
| | GGTCTATGATCTTGAGGGCAGACAGCAGAATTCCTCTTATAAAGAAAACTGTTT |
| | GGGAAAATACGTTGAGGGAGAGAAGACCTTGGGCCAAGATGCTAAATGGGAAT |
| | GCAAAGCTTGAGCTGCTCTGCAAGAGAAAATAAGCAGGACAGAGGATTTGCTC |
| | TGGACAGAGATGGAAGAGCCGGGAACAGAGAAGTGTGGGGAAGAGATAGGAA |
| | CCAGCAGGATGGCAGGGGCAAAGGGCTCAAGGGTGAGGAGGCCAGTGGGACC |
| | CCACAGAGTTGGGGAGATAAAGGAACATTGGTTGCTTTGGTGGCACGTAAGCT |
| | CCTTGTCTGTCTCCAGCACCCAGAATCTCATTAAAGCTTATTTATTGTACCTCCA |
| | GCGGCTGTGTGCAATGGGGTCTTTTGTGGAAATCAAGGAGCAGACAGGTTTCAT |
| | GTGTACTGTCACCACGTGGGATGGAACCAGAGGCATGGAAGCAAGACGCTAAA |
| | TGAAGAGGGCCATAAGGGCTGGGATTCCCAGGCACCTTAGGAACAGCTTGTCTT |
| | TTTTTTTTTCCTCTCCAAAAAAAATGTTTAAGGGACGGTGTCTCCTGTCACCCAG |
| | GCTGGAGTGCAATGGCACGATCATAGCTCATTGCAGCCTCTAACTCCGGGGCTC |
| | AAGCAATCCTCCCACCTCAGCCTACCAAGTAGCTGTGACCACAGCTGCCCCTCA |
| | CCATGCTAAGCTAATTTTTTTAATTAGATAGTACATAAACGTCCCAAAATTAGA |
| | AGATAAAAAGACATGAGGGATCCATTCTAATTTGTGTTTGGAGTGTAATGGTCC |
| | AGCTCCATTCTTCTGCACATGGATATCCAGTTTTACACAACACTGTGAATGTAAT |
| | GAATGCCACTGAATCATACACTCAAAAATAGCTAAAATGGCAAATTGTCTGTTA |
| | TCTCTTTTTAACCACCATTTTTGAAAATTAATTATACCAAAAAACCATTGAATAG |
| | TGCACTTTATTTATTTATTTATTTGTTTATTTATTTATTTATTTTAGAAATAAGAG |
| | TCTCACTTTGTTGCCCAGGCTGGAGTGCAGTGGCGTGATCATGGCTCATTGCAG |
| | CCTCGACCTGCTGGGCTCGGGCTATCCTTCCATCTCAGCCTCCCGAGTAGCTGG |
| | GACTATAGGTGGGCGCCACCCCACCTGGCTAAATCTCTTTTTAACTTTTGTAGA |
| | GATAGGCATCTCGCTATGTTGCCTAGGCTGGGCTGGAACTCCTGGGCTCAAGTG |
| | CTCCTCCTGCCTTGGCCTCCCAAAGCGCTAGGATTACAGATGTGAGCCACCGCG |
| | CCCACCCTGAACCTTACTTTTTTTGCTCAGTTTCTGGTAATTCAGAGAATGCCTC |
| | CTGAGTTGTTCTACACCCACCTCATATTCCATGGGAGGGCTGTACAGGGCTTTTT |
| | TAACGAGGCCTCTAAGGACAGGCATTTGTATCCTTTCCAGCCTTTCACTATTACA |
| | ATGTTGTAGTGAATAACTTTACACACTGTCATTTATTTTACTTTTTTTTTTTTTTA |
| | TTTTAGAGAAAGGAATCTTGCCATCTTGCCCAGGCTGGTCTCAAATTCCTGGGC |
| | CCAAACAATCCTCCCGCCTTGGCCTCCTAAAGTACTGGGATTTATAGGCATAAG |
| | CCACCGTGCCTGGCCAATGCACACTGTCATTTAGCTCATGTTAACACCTGAGTG |
| | TAGGACACACTCCTGGAGGTGGAATTGCTGGGCCAAAGAGTATGTTTCTTGTCA |
| | TTGTGATAGATATTGACAAATGAACCCTCACAGAAGTTGTGCTGAGTTCTGTTC |
| | CCACCAGCGACGTAGGCGATGACCTTTTTCTGGAGGGAGGGGGCATCCTTGGA |
| | GTCCACAGAGCCAGGAATGGAGAGTGGGCCCAGAATTTTGGTATAGGTGTTGT |
| | ATAAACTTATAGTAAGGTTAAGAAAACCGCAACTATCCTTATCAGAGACTTGGC |
| | GGGGGGCAGGGTATGATGGAGATCATAAGGAGGCTAAAACACTCCACACCCTC |
| | CCTCTGCATTGCTCCTGCACGGGAGTCGGGAATCTTTTCAGGTTGATACGATCTC |
| | ACCTTGAGGAGCTGTGAGGTCCCAGAAGCCTCTGGGTTGCAGATTGCTTGGGGT |
| | GAAAATGTCTGTGCTACTGAAATCTAACTTTTTACAAAAAATTACGGGCTGGGC |
| | GCAGTGGCTCACGCCTGTAATCCCAGCACTTTGGGAGGCTGCAGCGGGTGGATC |
| | ACTTGAGGTAAGGAGTTCAAGACCAGACCATAGTGAAACCGTGTCTCTACAAA |
| | AAAAATTAGCCAGGTGTGGTGGTGCATGCTTGTAATCCCAGCTACTCAGAAGGC |
| | TGAGGTGGGAGAATCCCTTGAACCCGGGAAGTGGAGGCTGGAGTAAACCATGA |
| | TCGAGTTACTGCACTCCAGCCTGGGTGACAAGAGTGAGACTCTGTCTCCAAAAA |
| | AAAAAAAAAAAAAAAAAAAACTGGATTGCCTGGCTCTACTCCGGGCACAGCAT |
| | GCAGGCCCAGTTCTGCTGCTCTGCTGTTTGTTCTGCTTTCCTCCACATATTGGCA |
| | TCACCCTCTGGTGCCAAGATGGCTGCTGCATTCCAGGCATCACATCCAGACTCA |
| | GACCCAGAGAAGCTGCCCATCCCTACCTGGGTGAGCCTTTGTAGGAACGAGAA |
| | ACCGCATCCAGCAGCAGAAACCTCACCCAGCAGCGTCTTTTCCGGTCTCATTCA |
| | CCAGCGCCGCCCACCGCTCAACCAATCCCTGGCCAAAAGAATGGGACCGCCTG |
| | GAAGGCTGGACCAAACAGGACCTGCCCTCTGGGGCTGGGGAGAGGCCCAGATG |
| | AAGGCTGCAGGACAGGATGGACTCCTAGACCTCTGTTACCAGCAGTGACTACCT |
| | CTGTCTGGGTGGTTGGAACATGTTTGAATTTTATTCTAAGTACTGTCTACAAGTT |
| | CTGCAATAAACCTTGACTCTTCTTTTAATAATGCAAAA (SEQ ID NO: 35) |
| | >NP_006496.4 poliovirus receptor isoform alpha precursor |
| | [Homo sapiens], amino acid sequence |
| | MARAMAAAWPLLLVALLVLSWPPPGTGDVVVQAPTQVPGFLGDSVTLPCYLQVP |
| | NMEVTHVSQLTWARHGESGSMAVFHQTQGPSYSESKRLEFVAARLGAELRNASLR |
| | MFGLRVEDEGNYTCLFVTFPQGSRSVDIWLRVLAKPQNTAEVQKVQLTGEPVPMA |
| | RCVSTGGRPPAQITWHSDLGGMPNTSQVPGFLSGTVTVTSLWILVPSSQVDGKNVT |
| | CKVEHESFEKPQLLTVNLTVYYPPEVSISGYDNNWYLGQNEATLTCDARSNPEPTG |
| | YNWSTTMGPLPPFAVAQGAQLLIRPVDKPINTTLICNVTNALGARQAELTVQVKEG |
| | PPSEHSGMSRNAIIFLVLGILVFLILLGIGIYFYWSKCSREVLWHCHLCPSSTEHASAS |
| | ANGHVSYSAVSRENSSSQDPQTEGTR (SEQ ID NO: 36) |
| Human | >NM_002856.3 Homo sapiens nectin cell adhesion molecule 2 |
| Nectin-2 | (NECTIN2), transcript variant alpha, mRNA, nucleic acid |
| (CD112) | sequence |
| isoform | GTGACGTCAGCGGGTTCGAACCGCCGGAGCTGAGCGAGAGGCCGGGGGTGCCG |
| alpha | AGCCGGGCGGGGAGAGCTGGGCCGGGAGAGCAGAACAGGGAGGCTAGAGCGC |
| | AGCGGGAACCGGCCCGGAGCCGGAGCCGGAGCCCCACAGGCACCTACTAAACC |
| | GCCCAGCCGATCGGCCCCCACAGAGTGGCCCGCGGGCCTCCGGCCGGGCCCAG |
| | TCCCCTCCCGGGCCCTCCATGGCCCGGGCCGCTGCCCTCCTGCCGTCGAGATCG |
| | CCGCCGACGCCGCTGCTGTGGCCGCTGCTGCTGCTGCTGCTCCTGGAAACCGGA |
| | GCCCAGGATGTGCGAGTTCAAGTGCTACCCGAGGTGCGAGGCCAGCTCGGGGG |
| | CACCGTGGAGCTGCCGTGCCACCTGCTGCCACCTGTTCCTGGACTGTACATCTC |
| | CCTGGTGACCTGGCAGCGCCCAGATGCACCTGCGAACCACCAGAATGTGGCCG |
| | CCTTCCACCCTAAGATGGGTCCCAGCTTCCCCAGCCCGAAGCCTGGCAGCGAGC |
| | GGCTGTCCTTCGTCTCTGCCAAGCAGAGCACTGGGCAAGACACAGAGGCAGAG |
| | CTCCAGGACGCCACGCTGGCCCTCCACGGGCTCACGGTGGAGGACGAGGGCAA |
| | CTACACTTGCGAGTTTGCCACCTTCCCCAAGGGGTCCGTCCGAGGGATGACCTG |
| | GCTCAGAGTCATAGCCAAGCCCAAGAACCAAGCTGAGGCCCAGAAGGTCACGT |
| | TCAGCCAGGACCCTACGACAGTGGCCCTCTGCATCTCCAAAGAGGGCCGCCCAC |
| | CTGCCCGGATCTCCTGGCTCTCATCCCTGGACTGGGAAGCCAAAGAGACTCAGG |
| | TGTCAGGGACCCTGGCCGGAACTGTCACTGTCACCAGCCGCTTCACCTTGGTGC |
| | CCTCGGGCCGAGCAGATGGTGTCACGGTCACCTGCAAAGTGGAGCATGAGAGC |
| | TTCGAGGAACCAGCCCTGATACCTGTGACCCTCTCTGTACGCTACCCTCCTGAA |
| | GTGTCCATCTCCGGCTATGATGACAACTGGTACCTCGGCCGTACTGATGCCACC |
| | CTGAGCTGTGACGTCCGCAGCAACCCAGAGCCCACGGGCTATGACTGGAGCAC |
| | GACCTCAGGCACCTTCCCGACCTCCGCAGTGGCCCAGGGCTCCCAGCTGGTCAT |
| | CCACGCAGTGGACAGTCTGTTCAATACCACCTTCGTCTGCACAGTCACCAATGC |
| | CGTGGGCATGGGCCGCGCTGAGCAGGTCATCTTTGTCCGAGAAACCCCCAGGG |
| | CCTCGCCCCGAGATGTGGGCCCGCTGGTGTGGGGGGCCGTGGGGGGGACACTG |
| | CTGGTGCTGCTGCTTCTGGCTGGGGGGTCCTTGGCCTTCATCCTGCTGAGGGTG |
| | AGGAGGAGGAGGAAGAGCCCTGGAGGAGCAGGAGGAGGAGCCAGTGGCGACG |
| | GGGGATTCTACGATCCGAAAGCTCAGGTGTTGGGAAATGGGGACCCCGTCTTCT |
| | GGACACCAGTAGTCCCTGGTCCCATGGAACCAGATGGCAAGGATGAGGAGGAG |
| | GAGGAGGAGGAAGAGAAGGCAGAGAAAGGCCTCATGTTGCCTCCACCCCCAGC |
| | ACTCGAGGATGACATGGAGTCCCAGCTGGACGGCTCCCTCATCTCACGGCGGGC |
| | AGTTTATGTGTGACCTGGACACAGACAGAGACAGAGCCAGGCCCGGCCCTCCC |
| | GCCCCCGACCTGACCACGCCGGCCTAGGGTTCCAGACTGGTTGGACTTGTTCGT |
| | CTGGACGACACTGGAGTGGAACACTGCCTCCCACTTTCTTGGGACTTGGAGGGA |
| | GGTGGAACAGCACACTGGACTTCTCCCGTCTCTAGGGCTGCATGGGGAGCCCGG |
| | GGAGCTGAGTAGTGGGGATCCAGAGAGGACCCCCGCCCCCAGAGACTTGGTTT |
| | TGGCTCCAGCCTTCCCCTGGCCCCGTGACACTCAGGAGTTAATAAATGCCTTGG |
| | AGGAAAACA (SEQ ID NO: 37) |
| | >NP_002847.1 nectin-2 isoform alpha precursor [Homo sapiens], |
| | amino acid sequence |
| | MARAAALLPSRSPPTPLLWPLLLLLLLETGAQDVRVQVLPEVRGQLGGTVELPCHL |
| | LPPVPGLYISLVTWQRPDAPANHQNVAAFHPKMGPSFPSPKPGSERLSFVSAKQSTG |
| | QDTEAELQDATLALHGLTVEDEGNYTCEFATFPKGSVRGMTWLRVIAKPKNQAEA |
| | QKVTFSQDPTTVALCISKEGRPPARISWLSSLDWEAKETQVSGTLAGTVTVTSRFTL |
| | VPSGRADGVTVTCKVEHESFEEPALIPVTLSVRYPPEVSISGYDDNWYLGRTDATLS |
| | CDVRSNPEPTGYDWSTTSGTFPTSAVAQGSQLVIHAVDSLFNTTFVCTVTNAVGMG |
| | RAEQVIFVRETPRASPRDVGPLVWGAVGGTLLVLLLLAGGSLAFILLRVRRRRKSP |
| | GGAGGGASGDGGFYDPKAQVLGNGDPVFWTPVVPGPMEPDGKDEEEEEEEEKAE |
| | KGLMLPPPPALEDDMESQLDGSLISRRAVYV (SEQ ID NO: 38) |
| Human | >NM_001042724.2 Homo sapiens nectin cell adhesion |
| Nectin-2 | molecule 2 (NECTIN2), transcript variant delta, mRNA, |
| (CD112) | nucleic acid sequence |
| isoform | GTGACGTCAGCGGGTTCGAACCGCCGGAGCTGAGCGAGAGGCCGGGGGTGCCG |
| delta | AGCCGGGCGGGGAGAGCTGGGCCGGGAGAGCAGAACAGGGAGGCTAGAGCGC |
| | AGCGGGAACCGGCCCGGAGCCGGAGCCGGAGCCCCACAGGCACCTACTAAACC |
| | GCCCAGCCGATCGGCCCCCACAGAGTGGCCCGCGGGCCTCCGGCCGGGCCCAG |
| | TCCCCTCCCGGGCCCTCCATGGCCCGGGCCGCTGCCCTCCTGCCGTCGAGATCG |
| | CCGCCGACGCCGCTGCTGTGGCCGCTGCTGCTGCTGCTGCTCCTGGAAACCGGA |
| | GCCCAGGATGTGCGAGTTCAAGTGCTACCCGAGGTGCGAGGCCAGCTCGGGGG |
| | CACCGTGGAGCTGCCGTGCCACCTGCTGCCACCTGTTCCTGGACTGTACATCTC |
| | CCTGGTGACCTGGCAGCGCCCAGATGCACCTGCGAACCACCAGAATGTGGCCG |
| | CCTTCCACCCTAAGATGGGTCCCAGCTTCCCCAGCCCGAAGCCTGGCAGCGAGC |
| | GGCTGTCCTTCGTCTCTGCCAAGCAGAGCACTGGGCAAGACACAGAGGCAGAG |
| | CTCCAGGACGCCACGCTGGCCCTCCACGGGCTCACGGTGGAGGACGAGGGCAA |
| | CTACACTTGCGAGTTTGCCACCTTCCCCAAGGGGTCCGTCCGAGGGATGACCTG |
| | GCTCAGAGTCATAGCCAAGCCCAAGAACCAAGCTGAGGCCCAGAAGGTCACGT |
| | TCAGCCAGGACCCTACGACAGTGGCCCTCTGCATCTCCAAAGAGGGCCGCCCAC |
| | CTGCCCGGATCTCCTGGCTCTCATCCCTGGACTGGGAAGCCAAAGAGACTCAGG |
| | TGTCAGGGACCCTGGCCGGAACTGTCACTGTCACCAGCCGCTTCACCTTGGTGC |
| | CCTCGGGCCGAGCAGATGGTGTCACGGTCACCTGCAAAGTGGAGCATGAGAGC |
| | TTCGAGGAACCAGCCCTGATACCTGTGACCCTCTCTGTACGCTACCCTCCTGAA |
| | GTGTCCATCTCCGGCTATGATGACAACTGGTACCTCGGCCGTACTGATGCCACC |
| | CTGAGCTGTGACGTCCGCAGCAACCCAGAGCCCACGGGCTATGACTGGAGCAC |
| | GACCTCAGGCACCTTCCCGACCTCCGCAGTGGCCCAGGGCTCCCAGCTGGTCAT |
| | CCACGCAGTGGACAGTCTGTTCAATACCACCTTCGTCTGCACAGTCACCAATGC |
| | CGTGGGCATGGGCCGCGCTGAGCAGGTCATCTTTGTCCGAGAGACCCCCAACAC |
| | AGCAGGCGCAGGGGCCACAGGCGGCATCATCGGGGGCATCATCGCCGCCATCA |
| | TTGCTACTGCTGTGGCTGCCACGGGCATCCTTATCTGCCGGCAGCAGCGGAAGG |
| | AGCAGACGCTGCAGGGGGCAGAGGAGGACGAAGACCTGGAGGGACCTCCCTCC |
| | TACAAGCCACCGACCCCAAAAGCGAAGCTGGAGGCACAGGAGATGCCCTCCCA |
| | GCTCTTCACTCTGGGGGCCTCGGAGCACAGCCCACTCAAGACCCCCTACTTTGA |
| | TGCTGGCGCCTCATGCACTGAGCAGGAAATGCCTCGATACCATGAGCTGCCCAC |
| | CTTGGAAGAACGGTCAGGACCCTTGCACCCTGGAGCCACAAGCCTGGGGTCCC |
| | CCATCCCGGTGCCTCCAGGGCCACCTGCTGTGGAAGACGTTTCCCTGGATCTAG |
| | AGGATGAGGAGGGGGAGGAGGAGGAAGAGTATCTGGACAAGATCAACCCCAT |
| | CTATGATGCTCTGTCCTATAGCAGCCCCTCTGATTCCTACCAGGGCAAAGGCTT |
| | TGTCATGTCCCGGGCCATGTATGTGTGAGCTGCCATGCGCCTGGCGTCTCACAT |
| | CTCACCTGTTGATCCCTTAGCTTTCTTGCCAAGGATCTAGTGCCCCCTGACCTCT |
| | GGCCAGGCCACTGTCAGTTAACACATATGCATTCCATTTGTGATGTCTACCTTG |
| | GTGGCTCCACTATGACCCCTAACCCATGAGCCCAGAGAAATTCACCGTGATAAT |
| | GGAATCCTGGCAACCTTATCTCATGAGGCAGGAGGTGGGGAAGGTGCTTCTGC |
| | ACAACCTCTGATCCCAAGGACTCCTCTCCCAGACTGTGACCTTAGACCATACCT |
| | CTCACCCCCCAATGCCTCGACTCCCCCAAAATCACAAAGAAGACCCTAGACCTA |
| | TAATTTGTCTTCAGGTAGTAAATTCCCAATAGGTCTGCTGGAGTGGGCGCTGAG |
| | GGCTCCCTGCTGCTCAGACCTGAGCCCTCCAGGCAGCAGGGTCCCACTTACCCC |
| | CTCCCCACCCTGTTCCCCAAAGGTGGGAAAGAGGGGATTCCCCAGCCCAAGGC |
| | AGGGTTTTCCCAGCACCCTCCTGTAAGCAGAAGTCTCAGGGTCCAGACCCTTCC |
| | CTGAGCCCCCACCCCCACCCCAATTCCTGCCTACCAAGCAAGCAGCCCCAGCCT |
| | AGGGTCAGACAGGGTGAGCCTCATACAGACTGTGCCTTGATGGCCCCAGCCTTG |
| | GGAGAAGAATTTACTGTTAACCTGGAAGACTACTGAATCATTTTACCCTTGCCC |
| | AGTGGAATAGGACCTAAACATCCCCCTTCCGGGGAAAGTGGGTCATCTGAATTG |
| | GGGGTAGCAATTGATACTGTTTTGTAAACTACATTTCCTACAAAATATGAATTT |
| | ATACTTTGA (SEQ ID NO: 39) |
| | >NP_001036189.1 nectin-2 isoform delta precursor |
| | [Homo sapiens], amino acid sequence |
| | MARAAALLPSRSPPTPLLWPLLLLLLLETGAQDVRVQVLPEVRGQLGGTVELPCHL |
| | LPPVPGLYISLVTWQRPDAPANHQNVAAFHPKMGPSFPSPKPGSERLSFVSAKQSTG |
| | QDTEAELQDATLALHGLTVEDEGNYTCEFATFPKGSVRGMTWLRVIAKPKNQAEA |
| | QKVTFSQDPTTVALCISKEGRPPARISWLSSLDWEAKETQVSGTLAGTVTVTSRFTL |
| | VPSGRADGVTVTCKVEHESFEEPALIPVTLSVRYPPEVSISGYDDNWYLGRTDATLS |
| | CDVRSNPEPTGYDWSTTSGTFPTSAVAQGSQLVIHAVDSLFNTTFVCTVTNAVGMG |
| | RAEQVIFVRETPNTAGAGATGGIIGGIIAAIIATAVAATGILICRQQRKEQTLQGAEE |
| | DEDLEGPPSYKPPTPKAKLEAQEMPSQLFTLGASEHSPLKTPYFDAGASCTEQEMPR |
| | YHELPTLEERSGPLHPGATSLGSPIPVPPGPPAVEDVSLDLEDEEGEEEEEYLDKINPI |
| | YDALSYSSPSDSYQGKGFVMSRAMYV (SEQ ID NO: 40) |
| Human IL-10 | >NM_000572.3 Homo sapiens interleukin 10 (IL10), |
| | transcript variant 1, mRNA, nucleic acid sequence |
| | ACACATCAGGGGCTTGCTCTTGCAAAACCAAACCACAAGACAGACTTGCAAAA |
| | GAAGGCATGCACAGCTCAGCACTGCTCTGTTGCCTGGTCCTCCTGACTGGGGTG |
| | AGGGCCAGCCCAGGCCAGGGCACCCAGTCTGAGAACAGCTGCACCCACTTCCC |
| | AGGCAACCTGCCTAACATGCTTCGAGATCTCCGAGATGCCTTCAGCAGAGTGAA |
| | GACTTTCTTTCAAATGAAGGATCAGCTGGACAACTTGTTGTTAAAGGAGTCCTT |
| | GCTGGAGGACTTTAAGGGTTACCTGGGTTGCCAAGCCTTGTCTGAGATGATCCA |
| | GTTTTACCTGGAGGAGGTGATGCCCCAAGCTGAGAACCAAGACCCAGACATCA |
| | AGGCGCATGTGAACTCCCTGGGGGAGAACCTGAAGACCCTCAGGCTGAGGCTA |
| | CGGCGCTGTCATCGATTTCTTCCCTGTGAAAACAAGAGCAAGGCCGTGGAGCAG |
| | GTGAAGAATGCCTTTAATAAGCTCCAAGAGAAAGGCATCTACAAAGCCATGAG |
| | TGAGTTTGACATCTTCATCAACTACATAGAAGCCTACATGACAATGAAGATACG |
| | AAACTGAGACATCAGGGTGGCGACTCTATAGACTCTAGGACATAAATTAGAGG |
| | TCTCCAAAATCGGATCTGGGGCTCTGGGATAGCTGACCCAGCCCCTTGAGAAAC |
| | CTTATTGTACCTCTCTTATAGAATATTTATTACCTCTGATACCTCAACCCCCATT |
| | TCTATTTATTTACTGAGCTTCTCTGTGAACGATTTAGAAAGAAGCCCAATATTAT |
| | AATTTTTTTCAATATTTATTATTTTCACCTGTTTTTAAGCTGTTTCCATAGGGTGA |
| | CACACTATGGTATTTGAGTGTTTTAAGATAAATTATAAGTTACATAAGGGAGGA |
| | AAAAAAATGTTCTTTGGGGAGCCAACAGAAGCTTCCATTCCAAGCCTGACCACG |
| | CTTTCTAGCTGTTGAGCTGTTTTCCCTGACCTCCCTCTAATTTATCTTGTCTCTGG |
| | GCTTGGGGCTTCCTAACTGCTACAAATACTCTTAGGAAGAGAAACCAGGGAGC |
| | CCCTTTGATGATTAATTCACCTTCCAGTGTCTCGGAGGGATTCCCCTAACCTCAT |
| | TCCCCAACCACTTCATTCTTGAAAGCTGTGGCCAGCTTGTTATTTATAACAACCT |
| | AAATTTGGTTCTAGGCCGGGCGCGGTGGCTCACGCCTGTAATCCCAGCACTTTG |
| | GGAGGCTGAGGCGGGTGGATCACTTGAGGTCAGGAGTTCCTAACCAGCCTGGT |
| | CAACATGGTGAAACCCCGTCTCTACTAAAAATACAAAAATTAGCCGGGCATGG |
| | TGGCGCGCACCTGTAATCCCAGCTACTTGGGAGGCTGAGGCAAGAGAATTGCTT |
| | GAACCCAGGAGATGGAAGTTGCAGTGAGCTGATATCATGCCCCTGTACTCCAGC |
| | CTGGGTGACAGAGCAAGACTCTGTCTCAAAAAATAAAAATAAAAATAAATTTG |
| | GTTCTAATAGAACTCAGTTTTAACTAGAATTTATTCAATTCCTCTGGGAATGTTA |
| | CATTGTTTGTCTGTCTTCATAGCAGATTTTAATTTTGAATAAATAAATGTATCTT |
| | ATTCACATCA (SEQ ID NO: 41) |
| | >NP_000563.1 interleukin-10 isoform 1 precursor |
| | [Homo sapiens], amino acid sequence |
| | MHSSALLCCLVLLTGVRASPGQGTQSENSCTHFPGNLPNMLRDLRDAFSRVKTFFQ |
| | MKDQLDNLLLKESLLEDFKGYLGCQALSEMIQFYLEEVMPQAENQDPDIKAHVNS |
| | LGENLKTLRLRLRRCHRFLPCENKSKAVEQVKNAFNKLQEKGIYKAMSEFDIFINYI |
| | EAYMTMKIRN (SEQ ID NO: 42) |
| Human | >NM_007115.3 Homo sapiens TNF alpha induced protein 6 |
| TSG-6 | (TNFAIP6), mRNA, nucleic amino acid sequence |
| | AGTCACATTTCAGCCACTGCTCTGAGAATTTGTGAGCAGCCCCTAACAGGCTGT |
| | TACTTCACTACAACTGACGATATGATCATCTTAATTTACTTATTTCTCTTGCTAT |
| | GGGAAGACACTCAAGGATGGGGATTCAAGGATGGAATTTTTCATAACTCCATAT |
| | GGCTTGAACGAGCAGCCGGTGTGTACCACAGAGAAGCACGGTCTGGCAAATAC |
| | AAGCTCACCTACGCAGAAGCTAAGGCGGTGTGTGAATTTGAAGGCGGCCATCT |
| | CGCAACTTACAAGCAGCTAGAGGCAGCCAGAAAAATTGGATTTCATGTCTGTGC |
| | TGCTGGATGGATGGCTAAGGGCAGAGTTGGATACCCCATTGTGAAGCCAGGGC |
| | CCAACTGTGGATTTGGAAAAACTGGCATTATTGATTATGGAATCCGTCTCAATA |
| | GGAGTGAAAGATGGGATGCCTATTGCTACAACCCACACGCAAAGGAGTGTGGT |
| | GGCGTCTTTACAGATCCAAAGCAAATTTTTAAATCTCCAGGCTTCCCAAATGAG |
| | TACGAAGATAACCAAATCTGCTACTGGCACATTAGACTCAAGTATGGTCAGCGT |
| | ATTCACCTGAGTTTTTTAGATTTTGACCTTGAAGATGACCCAGGTTGCTTGGCTG |
| | ATTATGTTGAAATATATGACAGTTACGATGATGTCCATGGCTTTGTGGGAAGAT |
| | ACTGTGGAGATGAGCTTCCAGATGACATCATCAGTACAGGAAATGTCATGACCT |
| | TGAAGTTTCTAAGTGATGCTTCAGTGACAGCTGGAGGTTTCCAAATCAAATATG |
| | TTGCAATGGATCCTGTATCCAAATCCAGTCAAGGAAAAAATACAAGTACTACTT |
| | CTACTGGAAATAAAAACTTTTTAGCTGGAAGATTTAGCCACTTATAAAAAAAAA |
| | AAAAAGGATGATCAAAACACACAGTGTTTATGTTGGAATCTTTTGGAACTCCTT |
| | TGATCTCACTGTTATTATTAACATTTATTTATTATTTTTCTAAATGTGAAAGCAA |
| | TACATAATTTAGGGAAAATTGGAAAATATAGGAAACTTTAAACGAGAAAATGA |
| | AACCTCTCATAATCCCACTGCATAGAAATAACAAGCGTTAACATTTTCATATTTT |
| | TTTCTTTCAGTCATTTTTCTATTTGTGGTATATGTATATATGTACCTATATGTATT |
| | TGCATTTGAAATTTTGGAATCCTGCTCTATGTACAGTTTTGTATTATACTTTTTA |
| | AATCTTGAACTTTATAAACATTTTCTGAAATCATTGATTATTCTACAAAAACATG |
| | ATTTTAAACAGCTGTAAAATATTCTATGATATGAATGTTTTATGCATTATTTAAG |
| | CCTGTCTCTATTGTTGGAATTTCAGGTCATTTTCATAAATATTGTTGCAATAAAT |
| | ATCCTTGAACACACAAAAAAAAAAAAAAAA (SEQ ID NO: 43) |
| | >NP_009046.2 tumor necrosis factor-inducible gene 6 |
| | protein precursor [Homo sapiens, amino acid sequence] |
| | MIILIYLFLLLWEDTQGWGFKDGIFHNSIWLERAAGVYHREARSGKYKLTYAEAKA |
| | VCEFEGGHLATYKQLEAARKIGFHVCAAGWMAKGRVGYPIVKPGPNCGFGKTGII |
| | DYGIRLNRSERWDAYCYNPHAKECGGVFTDPKQIFKSPGFPNEYEDNQICYWHIRL |
| | KYGQRIHLSFLDFDLEDDPGCLADYVEIYDSYDDVHGFVGRYCGDELPDDIISTGN |
| | VMTLKFLSDASVTAGGFQIKYVAMDPVSKSSQGKNTSTTSTGNKNFLAGRFSHL |
| | (SEQ ID NO: 44) |
| Human | >NM_001024736.2 Homo sapiens CD276 molecule (CD276), |
| B7-H3 | transcript variant 1, mRNA, nucleic acid sequence |
| (CD276) | ATTCGGGCCGGGCCTCGCTGCGGCGGCGACTGAGCCAGGCTGGGCCGCGTCCCT |
| | GAGTCCCAGAGTCGGCGCGGCGCGGCAGGGGCAGCCTTCCACCACGGGGAGCC |
| | CAGCTGTCAGCCGCCTCACAGGAAGATGCTGCGTCGGCGGGGCAGCCCTGGCA |
| | TGGGTGTGCATGTGGGTGCAGCCCTGGGAGCACTGTGGTTCTGCCTCACAGGAG |
| | CCCTGGAGGTCCAGGTCCCTGAAGACCCAGTGGTGGCACTGGTGGGCACCGAT |
| | GCCACCCTGTGCTGCTCCTTCTCCCCTGAGCCTGGCTTCAGCCTGGCACAGCTCA |
| | ACCTCATCTGGCAGCTGACAGATACCAAACAGCTGGTGCACAGCTTTGCTGAGG |
| | GCCAGGACCAGGGCAGCGCCTATGCCAACCGCACGGCCCTCTTCCCGGACCTGC |
| | TGGCACAGGGCAACGCATCCCTGAGGCTGCAGCGCGTGCGTGTGGCGGACGAG |
| | GGCAGCTTCACCTGCTTCGTGAGCATCCGGGATTTCGGCAGCGCTGCCGTCAGC |
| | CTGCAGGTGGCCGCTCCCTACTCGAAGCCCAGCATGACCCTGGAGCCCAACAA |
| | GGACCTGCGGCCAGGGGACACGGTGACCATCACGTGCTCCAGCTACCAGGGCT |
| | ACCCTGAGGCTGAGGTGTTCTGGCAGGATGGGCAGGGTGTGCCCCTGACTGGC |
| | AACGTGACCACGTCGCAGATGGCCAACGAGCAGGGCTTGTTTGATGTGCACAG |
| | CATCCTGCGGGTGGTGCTGGGTGCAAATGGCACCTACAGCTGCCTGGTGCGCAA |
| | CCCCGTGCTGCAGCAGGATGCGCACAGCTCTGTCACCATCACACCCCAGAGAA |
| | GCCCCACAGGAGCCGTGGAGGTCCAGGTCCCTGAGGACCCGGTGGTGGCCCTA |
| | GTGGGCACCGATGCCACCCTGCGCTGCTCCTTCTCCCCCGAGCCTGGCTTCAGC |
| | CTGGCACAGCTCAACCTCATCTGGCAGCTGACAGACACCAAACAGCTGGTGCA |
| | CAGTTTCACCGAAGGCCGGGACCAGGGCAGCGCCTATGCCAACCGCACGGCCC |
| | TCTTCCCGGACCTGCTGGCACAAGGCAATGCATCCCTGAGGCTGCAGCGCGTGC |
| | GTGTGGCGGACGAGGGCAGCTTCACCTGCTTCGTGAGCATCCGGGATTTCGGCA |
| | GCGCTGCCGTCAGCCTGCAGGTGGCCGCTCCCTACTCGAAGCCCAGCATGACCC |
| | TGGAGCCCAACAAGGACCTGCGGCCAGGGGACACGGTGACCATCACGTGCTCC |
| | AGCTACCGGGGCTACCCTGAGGCTGAGGTGTTCTGGCAGGATGGGCAGGGTGT |
| | GCCCCTGACTGGCAACGTGACCACGTCGCAGATGGCCAACGAGCAGGGCTTGT |
| | TTGATGTGCACAGCGTCCTGCGGGTGGTGCTGGGTGCGAATGGCACCTACAGCT |
| | GCCTGGTGCGCAACCCCGTGCTGCAGCAGGATGCGCACGGCTCTGTCACCATCA |
| | CAGGGCAGCCTATGACATTCCCCCCAGAGGCCCTGTGGGTGACCGTGGGGCTGT |
| | CTGTCTGTCTCATTGCACTGCTGGTGGCCCTGGCTTTCGTGTGCTGGAGAAAGAT |
| | CAAACAGAGCTGTGAGGAGGAGAATGCAGGAGCTGAGGACCAGGATGGGGAG |
| | GGAGAAGGCTCCAAGACAGCCCTGCAGCCTCTGAAACACTCTGACAGCAAAGA |
| | AGATGATGGACAAGAAATAGCCTGACCATGAGGACCAGGGAGCTGCTACCCCT |
| | CCCTACAGCTCCTACCCTCTGGCTGCAATGGGGCTGCACTGTGAGCCCTGCCCC |
| | CAACAGATGCATCCTGCTCTGACAGGTGGGCTCCTTCTCCAAAGGATGCGATAC |
| | ACAGACCACTGTGCAGCCTTATTTCTCCAATGGACATGATTCCCAAGTCATCCT |
| | GCTGCCTTTTTTCTTATAGACACAATGAACAGACCACCCACAACCTTAGTTCTCT |
| | AAGTCATCCTGCCTGCTGCCTTATTTCACAGTACATACATTTCTTAGGGACACAG |
| | TACACTGACCACATCACCACCCTCTTCTTCCAGTGCTGCGTGGACCATCTGGCT |
| | GCCTTTTTTCTCCAAAAGATGCAATATTCAGACTGACTGACCCCCTGCCTTATTT |
| | CACCAAAGACACGATGCATAGTCACCCCGGCCTTGTTTCTCCAATGGCCGTGAT |
| | ACACTAGTGATCATGTTCAGCCCTGCTTCCACCTGCATAGAATCTTTTCTTCTCA |
| | GACAGGGACAGTGCGGCCTCAACATCTCCTGGAGTCTAGAAGCTGTTTCCTTTC |
| | CCCTCCTTCCTCCTCTTGCTCTAGCCTTAATACTGGCCTTTTCCCTCCCTGCCCCA |
| | AGTGAAGACAGGGCACTCTGCGCCCACCACATGCACAGCTGTGCATGGAGACC |
| | TGCAGGTGCACGTGCTGGAACACGTGTGGTTCCCCCCTGGCCCAGCCTCCTCTG |
| | CAGTGCCCCTCTCCCCTGCCCATCCTCCCCACGGAAGCATGTGCTGGTCACACT |
| | GGTTCTCCAGGGGTCTGTGATGGGGCCCCTGGGGGTCAGCTTCTGTCCCTCTGC |
| | CTTCTCACCTCTTTGTTCCTTTCTTTTCATGTATCCATTCAGTTGATGTTTATTGA |
| | GCAACTACAGATGTCAGCACTGTGTTAGGTGCTGGGGGCCCTGCGTGGGAAGA |
| | TAAAGTTCCTCCCTCAAGGACTCCCCATCCAGCTGGGAGACAGACAACTAACTA |
| | CACTGCACCCTGCGGTTTGCAGGGGGCTCCTGCCTGGCTCCCTGCTCCACACCT |
| | CCTCTGTGGCTCAAGGCTTCCTGGATACCTCACCCCCATCCCACCCATAATTCTT |
| | ACCCAGAGCATGGGGTTGGGGCGGAAACCTGGAGAGAGGGACATAGCCCCTCG |
| | CCACGGCTAGAGAATCTGGTGGTGTCCAAAATGTCTGTCCAGGTGTGGGCAGGT |
| | GGGCAGGCACCAAGGCCCTCTGGACCTTTCATAGCAGCAGAAAAGGCAGAGCC |
| | TGGGGCAGGGCAGGGCCAGGAATGCTTTGGGGACACCGAGGGGACTGCCCCCC |
| | ACCCCCACCATGGTGCTATTCTGGGGCTGGGGCAGTCTTTTCCTGGCTTGCCTCT |
| | GGCCAGCTCCTGGCCTCTGGTAGAGTGAGACTTCAGACGTTCTGATGCCTTCCG |
| | GATGTCATCTCTCCCTGCCCCAGGAATGGAAGATGTGAGGACTTCTAATTTAAA |
| | TGTGGGACTCGGAGGGATTTTGTAAACTGGGGGTATATTTTGGGGAAAATAAAT |
| | GTCTTTGTAAAAA (SEQ ID NO: 45) |
| | >NP_001019907.1 CD276 antigen isoform a precursor |
| | [Homo sapiens], amino acid sequence |
| | MLRRRGSPGMGVHVGAALGALWFCLTGALEVQVPEDPVVALVGTDATLCCSFSP |
| | EPGFSLAQLNLIWQLTDTKQLVHSFAEGQDQGSAYANRTALFPDLLAQGNASLRL |
| | QRVRVADEGSFTCFVSIRDFGSAAVSLQVAAPYSKPSMTLEPNKDLRPGDTVTITCS |
| | SYQGYPEAEVFWQDGQGVPLTGNVTTSQMANEQGLFDVHSILRVVLGANGTYSCL |
| | VRNPVLQQDAHSSVTITPQRSPTGAVEVQVPEDPVVALVGTDATLRCSFSPEPGFSL |
| | AQLNLIWQLTDTKQLVHSFTEGRDQGSAYANRTALFPDLLAQGNASLRLQRVRVA |
| | DEGSFTCFVSIRDFGSAAVSLQVAAPYSKPSMTLEPNKDLRPGDTVTITCSSYRGYP |
| | EAEVFWQDGQGVPLTGNVTTSQMANEQGLFDVHSVLRVVLGANGTYSCLVRNPV |
| | LQQDAHGSVTITGQPMTFPPEALWVTVGLSVCLIALLVALAFVCWRKIKQSCEEEN |
| | AGAEDQDGEGEGSKTALQPLKHSDSKEDDGQEIA (SEQ ID NO: 46) |
| Human | >NM_024626.4 Homo sapiens V-set domain containing |
| B7-H4 | T cell activation inhibitor 1 (VTCN1), transcript |
| (VTCN1) | variant 1, mRNA, nucleic acid sequence |
| | GTGAGTCACCAAGGAAGGCAGCGGCAGCTCCACTCAGCCAGTACCCAGATACG |
| | CTGGGAACCTTCCCCAGCCATGGCTTCCCTGGGGCAGATCCTCTTCTGGAGCAT |
| | AATTAGCATCATCATTATTCTGGCTGGAGCAATTGCACTCATCATTGGCTTTGGT |
| | ATTTCAGGGAGACACTCCATCACAGTCACTACTGTCGCCTCAGCTGGGAACATT |
| | GGGGAGGATGGAATCCTGAGCTGCACTTTTGAACCTGACATCAAACTTTCTGAT |
| | ATCGTGATACAATGGCTGAAGGAAGGTGTTTTAGGCTTGGTCCATGAGTTCAAA |
| | GAAGGCAAAGATGAGCTGTCGGAGCAGGATGAAATGTTCAGAGGCCGGACAGC |
| | AGTGTTTGCTGATCAAGTGATAGTTGGCAATGCCTCTTTGCGGCTGAAAAACGT |
| | GCAACTCACAGATGCTGGCACCTACAAATGTTATATCATCACTTCTAAAGGCAA |
| | GGGGAATGCTAACCTTGAGTATAAAACTGGAGCCTTCAGCATGCCGGAAGTGA |
| | ATGTGGACTATAATGCCAGCTCAGAGACCTTGCGGTGTGAGGCTCCCCGATGGT |
| | TCCCCCAGCCCACAGTGGTCTGGGCATCCCAAGTTGACCAGGGAGCCAACTTCT |
| | CGGAAGTCTCCAATACCAGCTTTGAGCTGAACTCTGAGAATGTGACCATGAAGG |
| | TTGTGTCTGTGCTCTACAATGTTACGATCAACAACACATACTCCTGTATGATTGA |
| | AAATGACATTGCCAAAGCAACAGGGGATATCAAAGTGACAGAATCGGAGATCA |
| | AAAGGCGGAGTCACCTACAGCTGCTAAACTCAAAGGCTTCTCTGTGTGTCTCTT |
| | CTTTCTTTGCCATCAGCTGGGCACTTCTGCCTCTCAGCCCTTACCTGATGCTAAA |
| | ATAATGTGCCTCGGCCACAAAAAAGCATGCAAAGTCATTGTTACAACAGGGAT |
| | CTACAGAACTATTTCACCACCAGATATGACCTAGTTTTATATTTCTGGGAGGAA |
| | ATGAATTCATATCTAGAAGTCTGGAGTGAGCAAACAAGAGCAAGAAACAAAAA |
| | GAAGCCAAAAGCAGAAGGCTCCAATATGAACAAGATAAATCTATCTTCAAAGA |
| | CATATTAGAAGTTGGGAAAATAATTCATGTGAACTAGACAAGTGTGTTAAGAGT |
| | GATAAGTAAAATGCACGTGGAGACAAGTGCATCCCCAGATCTCAGGGACCTCC |
| | CCCTGCCTGTCACCTGGGGAGTGAGAGGACAGGATAGTGCATGTTCTTTGTCTC |
| | TGAATTTTTAGTTATATGTGCTGTAATGTTGCTCTGAGGAAGCCCCTGGAAAGT |
| | CTATCCCAACATATCCACATCTTATATTCCACAAATTAAGCTGTAGTATGTACCC |
| | TAAGACGCTGCTAATTGACTGCCACTTCGCAACTCAGGGGCGGCTGCATTTTAG |
| | TAATGGGTCAAATGATTCACTTTTTATGATGCTTCCAAAGGTGCCTTGGCTTCTC |
| | TTCCCAACTGACAAATGCCAAAGTTGAGAAAAATGATCATAATTTTAGCATAAA |
| | CAGAGCAGTCGGCGACACCGATTTTATAAATAAACTGAGCACCTTCTTTTTAAA |
| | CAAACAAATGCGGGTTTATTTCTCAGATGATGTTCATCCGTGAATGGTCCAGGG |
| | AAGGACCTTTCACCTTGTCTATATGGCATTATGTCATCACAAGCTCTGAGGCTTC |
| | TCCTTTCCATCCTGCGTGGACAGCTAAGACCTCAGTTTTCAATAGCATCTAGAG |
| | CAGTGGGACTCAGCTGGGGTGATTTCGCCCCCCATCTCCGGGGGAATGTCTGAA |
| | GACAATTTTGGTTACCTCAATGAGGGAGTGGAGGAGGATACAGTGCTACTACC |
| | AACTAGTGGATAGAGGCCAGGGATGCTGCTCAACCTCCTACCATGTACAGGAC |
| | GTCTCCCCATTACAACTACCCAATCCGAAGTGTCAACTGTGTCAGGGCTAAGAA |
| | ACCCTGGTTTTGAGTAGAAAAGGGCCTGGAAAGAGGGGAGCCAACAAATCTGT |
| | CTGCTTCCTCACATTAGTCATTGGCAAATAAGCATTCTGTCTCTTTGGCTGCTGC |
| | CTCAGCACAGAGAGCCAGAACTCTATCGGGCACCAGGATAACATCTCTCAGTG |
| | AACAGAGTTGACAAGGCCTATGGGAAATGCCTGATGGGATTATCTTCAGCTTGT |
| | TGAGCTTCTAAGTTTCTTTCCCTTCATTCTACCCTGCAAGCCAAGTTCTGTAAGA |
| | GAAATGCCTGAGTTCTAGCTCAGGTTTTCTTACTCTGAATTTAGATCTCCAGACC |
| | CTGCCTGGCCACAATTCAAATTAAGGCAACAAACATATACCTTCCATGAAGCAC |
| | ACACAGACTTTTGAAAGCAAGGACAATGACTGCTTGAATTGAGGCCTTGAGGA |
| | ATGAAGCTTTGAAGGAAAAGAATACTTTGTTTCCAGCCCCCTTCCCACACTCTT |
| | CATGTGTTAACCACTGCCTTCCTGGACCTTGGAGCCACGGTGACTGTATTACAT |
| | GTTGTTATAGAAAACTGATTTTAGAGTTCTGATCGTTCAAGAGAATGATTAAAT |
| | ATACATTTCCTACACCA (SEQ ID NO: 47) |
| | >NP_078902.2 V-set domain-containing T-cell activation |
| | inhibitor 1 isoform 1 precursor [Homo sapiens], amino |
| | acid sequence |
| | MASLGQILFWSIISIIIILAGAIALIIGFGISGRHSITVTTVASAGNIGEDGILSCTFEPDIK |
| | LSDIVIQWLKEGVLGLVHEFKEGKDELSEQDEMFRGRTAVFADQVIVGNASLRLKN |
| | VQLTDAGTYKCYIITSKGKGNANLEYKTGAFSMPEVNVDYNASSETLRCEAPRWF |
| | PQPTVVWASQVDQGANFSEVSNTSFELNSENVTMKVVSVLYNVTINNTYSCMIEN |
| | DIAKATGDIKVTESEIKRRSHLQLLNSKASLCVSSFFAISWALLPLSPYLMLK |
| | (SEQ ID NO: 48) |
| Human | >NM_022153.2 Homo sapiens V-set immunoregulatory |
| B7-H5 | receptor (VSIR), mRNA, nucleic acid sequence |
| (VISTA) | AGTCGCGGGAGGCTTCCCCGCGCCGGCCGCGTCCCGCCCGCTCCCCGGCACCAG |
| | AAGTTCCTCTGCGCGTCCGACGGCGACATGGGCGTCCCCACGGCCCTGGAGGCC |
| | GGCAGCTGGCGCTGGGGATCCCTGCTCTTCGCTCTCTTCCTGGCTGCGTCCCTAG |
| | GTCCGGTGGCAGCCTTCAAGGTCGCCACGCCGTATTCCCTGTATGTCTGTCCCG |
| | AGGGGCAGAACGTCACCCTCACCTGCAGGCTCTTGGGCCCTGTGGACAAAGGG |
| | CACGATGTGACCTTCTACAAGACGTGGTACCGCAGCTCGAGGGGCGAGGTGCA |
| | GACCTGCTCAGAGCGCCGGCCCATCCGCAACCTCACGTTCCAGGACCTTCACCT |
| | GCACCATGGAGGCCACCAGGCTGCCAACACCAGCCACGACCTGGCTCAGCGCC |
| | ACGGGCTGGAGTCGGCCTCCGACCACCATGGCAACTTCTCCATCACCATGCGCA |
| | ACCTGACCCTGCTGGATAGCGGCCTCTACTGCTGCCTGGTGGTGGAGATCAGGC |
| | ACCACCACTCGGAGCACAGGGTCCATGGTGCCATGGAGCTGCAGGTGCAGACA |
| | GGCAAAGATGCACCATCCAACTGTGTGGTGTACCCATCCTCCTCCCAGGATAGT |
| | GAAAACATCACGGCTGCAGCCCTGGCTACGGGTGCCTGCATCGTAGGAATCCTC |
| | TGCCTCCCCCTCATCCTGCTCCTGGTCTACAAGCAAAGGCAGGCAGCCTCCAAC |
| | CGCCGTGCCCAGGAGCTGGTGCGGATGGACAGCAACATTCAAGGGATTGAAAA |
| | CCCCGGCTTTGAAGCCTCACCACCTGCCCAGGGGATACCCGAGGCCAAAGTCA |
| | GGCACCCCCTGTCCTATGTGGCCCAGCGGCAGCCTTCTGAGTCTGGGCGGCATC |
| | TGCTTTCGGAGCCCAGCACCCCCCTGTCTCCTCCAGGCCCCGGAGACGTCTTCTT |
| | CCCATCCCTGGACCCTGTCCCTGACTCTCCAAACTTTGAGGTCATCTAGCCCAG |
| | CTGGGGGACAGTGGGCTGTTGTGGCTGGGTCTGGGGCAGGTGCATTTGAGCCA |
| | GGGCTGGCTCTGTGAGTGGCCTCCTTGGCCTCGGCCCTGGTTCCCTCCCTCCTGC |
| | TCTGGGCTCAGATACTGTGACATCCCAGAAGCCCAGCCCCTCAACCCCTCTGGA |
| | TGCTACATGGGGATGCTGGACGGCTCAGCCCCTGTTCCAAGGATTTTGGGGTGC |
| | TGAGATTCTCCCCTAGAGACCTGAAATTCACCAGCTACAGATGCCAAATGACTT |
| | ACATCTTAAGAAGTCTCAGAACGTCCAGCCCTTCAGCAGCTCTCGTTCTGAGAC |
| | ATGAGCCTTGGGATGTGGCAGCATCAGTGGGACAAGATGGACACTGGGCCACC |
| | CTCCCAGGCACCAGACACAGGGCACGGTGGAGAGACTTCTCCCCCGTGGCCGC |
| | CTTGGCTCCCCCGTTTTGCCCGAGGCTGCTCTTCTGTCAGACTTCCTCTTTGTAC |
| | CACAGTGGCTCTGGGGCCAGGCCTGCCTGCCCACTGGCCATCGCCACCTTCCCC |
| | AGCTGCCTCCTACCAGCAGTTTCTCTGAAGATCTGTCAACAGGTTAAGTCAATC |
| | TGGGGCTTCCACTGCCTGCATTCCAGTCCCCAGAGCTTGGTGGTCCCGAAACGG |
| | GAAGTACATATTGGGGCATGGTGGCCTCCGTGAGCAAATGGTGTCTTGGGCAAT |
| | CTGAGGCCAGGACAGATGTTGCCCCACCCACTGGAGATGGTGCTGAGGGAGGT |
| | GGGTGGGGCCTTCTGGGAAGGTGAGTGGAGAGGGGCACCTGCCCCCCGCCCTC |
| | CCCATCCCCTACTCCCACTGCTCAGCGCGGGCCATTGCAAGGGTGCCACACAAT |
| | GTCTTGTCCACCCTGGGACACTTCTGAGTATGAAGCGGGATGCTATTAAAAACT |
| | ACATGGGGAAACAGGTGCAAACCCTGGAGATGGATTGTAAGAGCCAGTTTAAA |
| | TCTGCACTCTGCTGCTCCTCCCCCACCCCCACCTTCCACTCCATACAATCTGGGC |
| | CTGGTGGAGTCTTCGCTTCAGAGCCATTCGGCCAGGTGCGGGTGATGTTCCCAT |
| | CTCCTGCTTGTGGGCATGCCCTGGCTTTGTTTTTATACACATAGGCAAGGTGAGT |
| | CCTCTGTGGAATTGTGATTGAAGGATTTTAAAGCAGGGGAGGAGAGTAGGGGG |
| | CATCTCTGTACACTCTGGGGGTAAAACAGGGAAGGCAGTGCCTGAGCATGGGG |
| | ACAGGTGAGGTGGGGCTGGGCAGACCCCCTGTAGCGTTTAGCAGGATGGGGGC |
| | CCCAGGTACTGTGGAGAGCATAGTCCAGCCTGGGCATTTGTCTCCTAGCAGCCT |
| | ACACTGGCTCTGCTGAGCTGGGCCTGGGTGCTGAAAGCCAGGATTTGGGGCTAG |
| | GCGGGAAGATGTTCGCCCAATTGCTTGGGGGGTTGGGGGGATGGAAAAGGGGA |
| | GCACCTCTAGGCTGCCTGGCAGCAGTGAGCCCTGGGCCTGTGGCTACAGCCAGG |
| | GAACCCCACCTGGACACATGGCCCTGCTTCTAAGCCCCCCAGTTAGGCCCAAAG |
| | GAATGGTCCACTGAGGGCCTCCTGCTCTGCCTGGGCTGGGCCAGGGGCTTTGAG |
| | GAGAGGGTAAACATAGGCCCGGAGATGGGGCTGACACCTCGAGTGGCCAGAAT |
| | ATGCCCAAACCCCGGCTTCTCCCTTGTCCCTAGGCAGAGGGGGGTCCCTTCTTTT |
| | GTTCCCTCTGGTCACCACAATGCTTGATGCCAGCTGCCATAGGAAGAGGGTGCT |
| | GGCTGGCCATGGTGGCACACACCTGTCCTCCCAGCACTTTGCAGGGCTGAGGTG |
| | GAAGGACCGCTTAAGCCCAGGTGTTCAAGGCTGCTGTGAGCTGTGTTCGAGCCA |
| | CTACACTCCAGCCTGGGGACGGAGCAAAACTTTGCCTCAAAACAAATTTTAAAA |
| | AGAAAGAAAGAAGGAAAGAGGGTATGTTTTTCACAATTCATGGGGGCCTGCAT |
| | GGCAGGAGTGGGGACAGGACACCTGCTGTTCCTGGAGTCGAAGGACAAGCCCA |
| | CAGCCCAGATTCCGGTTCTCCCAACTCAGGAAGAGCATGCCCTGCCCTCTGGGG |
| | AGGCTGGCCTGGCCCCAGCCCTCAGCTGCTGACCTTGAGGCAGAGACAACTTCT |
| | AAGAATTTGGCTGCCAGACCCCAGGCCTGGCTGCTGCTGTGTGGAGAGGGAGG |
| | CGGCCCGCAGCAGAACAGCCACCGCACTTCCTCCTCAGCTTCCTCTGGTGCGGC |
| | CCTGCCCTCTCTTCTCTGGACCCTTTTACAACTGAACGCATCTGGGCTTCGTGGT |
| | TTCCTGTTTTCAGCGAAATTTACTCTGAGCTCCCAGTTCCATCTTCATCCATGGC |
| | CACAGGCCCTGCCTACAACGCACTAGGGACGTCCCTCCCTGCTGCTGCTGGGGA |
| | GGGGCAGGCTGCTGGAGCCGCCCTCTGAGTTGCCCGGGATGGTAGTGCCTCTGA |
| | TGCCAGCCCTGGTGGCTGTGGGCTGGGGTGCATGGGAGAGCTGGGTGCGAGAA |
| | CATGGCGCCTCCAGGGGGGGGAGGAGCACTAGGGGCTGGGGCAGGAGGCTCC |
| | TGGAGCGCTGGATTCGTGGCACAGTCTGAGGCCCTGAGAGGGAAATCCATGCTT |
| | TTAAGAACTAATTCATTGTTAGGAGATCAATCAGGAATTAGGGGCCATCTTACC |
| | TATCTCCTGACATTCACAGTTTAATAGAGACTTCCTGCCTTTATTCCCTCCCAGG |
| | GAGAGGCTGAAGGAATGGAATTGAAAGCACCATTTGGAGGGTTTTGCTGACAC |
| | AGCGGGGACTGCTCAGCACTCCCTAAAAACACACCATGGAGGCCACTGGTGAC |
| | TGCTGGTGGGCAGGCTGGCCCTGCCTGGGGGAGTCCGTGGCGATGGGCGCTGG |
| | GGTGGAGGTGCAGGAGCCCCAGGACCTGCTTTTCAAAAGACTTCTGCCTGACCA |
| | GAGCTCCCACTACATGCAGTGGCCCAGGGCAGAGGGGCTGATACATGGCCTTTT |
| | TCAGGGGGTGCTCCTCGCGGGGTGGACTTGGGAGTGTGCAGTGGGACAGGGGG |
| | CTGCAGGGGTCCTGCCACCACCGAGCACCAACTTGGCCCCTGGGGTCCTGCCTC |
| | ATGAATGAGGCCTTCCCCAGGGCTGGCCTGACTGTGCTGGGGGCTGGGTTAACG |
| | TTTTCTCAGGGAACCACAATGCACGAAAGAGGAACTGGGGTTGCTAACCAGGA |
| | TGCTGGGAACAAAGGCCTCTTGAAGCCCAGCCACAGCCCAGCTGAGCATGAGG |
| | CCCAGCCCATAGACGGCACAGGCCACCTGGCCCATTCCCTGGGCATTCCCTGCT |
| | TTGCATTGCTGCTTCTCTTCACCCCATGGAGGCTATGTCACCCTAACTATCCTGG |
| | AATGTGTTGAGAGGGATTCTGAATGATCAATATAGCTTGGTGAGACAGTGCCGA |
| | GATAGATAGCCATGTCTGCCTTGGGCACGGGAGAGGGAAGTGGCAGCATGCAT |
| | GCTGTTTCTTGGCCTTTTCTGTTAGAATACTTGGTGCTTTCCAACACACTTTCAC |
| | ATGTGTTGTAACTTGTTTGATCCACCCCCTTCCCTGAAAATCCTGGGAGGTTTTA |
| | TTGCTGCCATTTAACACAGAGGGCAATAGAGGTTCTGAAAGGTCTGTGTCTTGT |
| | CAAAACAAGTAAACGGTGGAACTACGACTAAA |
| | (SEQ ID NO: 49) |
| | >NP_071436.1 V-type immunoglobulin domain-containing |
| | suppressor of T-cell activation precursor [Homo sapiens], |
| | amino acid sequence |
| | MGVPTALEAGSWRWGSLLFALFLAASLGPVAAFKVATPYSLYVCPEGQNVTLTCR |
| | LLGPVDKGHDVTFYKTWYRSSRGEVQTCSERRPIRNLTFQDLHLHHGGHQAANTS |
| | HDLAQRHGLESASDHHGNFSITMRNLTLLDSGLYCCLVVEIRHHHSEHRVHGAME |
| | LQVQTGKDAPSNCVVYPSSSQDSENITAAALATGACIVGILCLPLILLLVYKQRQAA |
| | SNRRAQELVRMDSNIQGIENPGFEASPPAQGIPEAKVRHPLSYVAQRQPSESGRHLL |
| | SEPSTPLSPPGPGDVFFPSLDPVPDSPNFEVI |
| | (SEQ ID NO: 50) |
| Human | >NM_007072.4 Homo sapiens HERV-H LTR-associating 2 (HHLA2), |
| B7-H7 | transcript variant 1, mRNA, nucleic acid sequence |
| (HHLA2, | AGTTCTCTTCAAGTCATGTAATCGACTTTTTTGAATTAGTTTTCAGTTTCATTTTG |
| CD28H) | TTTTCCCTAATTCAAGTTGGGAACACTTCATTTTCCCCAATTCAAGTTGGGAACA |
| | CTTCCTTGGTATTTCCTTGCTACATGGACTTTAGCAAATGCTACTTTACTCTCCTT |
| | CCAGCTACTCAGGAGGCTGAGGCAGGAGAATCGCTTGAACCCGGGAGGCGGAG |
| | GTTACAGTGAGCCTTTTCCTAGTTTTACTGTTGGAAGCCTAACTCACAGGAGAG |
| | ATTATGCAATACAGTCCTGAAGTCAAGGGAGGAGAGCATGTAGGAGAATACTA |
| | ACCCTGCACAGATTGTGATGGTGATGTGGAATATACTAAAGCCTAGAACGCACC |
| | TCCTCTGCATGACTAATATGTTCTGCACAAGACATGAAGGCACAGACAGCACTG |
| | TCTTTCTTCCTCATTCTCATAACATCTCTGAGTGGATCTCAAGGCATATTCCCTT |
| | TGGCTTTCTTCATTTATGTTCCTATGAATGAACAAATCGTCATTGGAAGACTTGA |
| | TGAAGATATAATTCTCCCTTCTTCATTTGAGAGGGGATCCGAAGTCGTAATACA |
| | CTGGAAGTATCAAGATAGCTATAAGGTTCACAGTTACTACAAAGGCAGTGACC |
| | ATTTGGAAAGCCAAGATCCCAGATATGCAAACAGGACATCCCTTTTCTATAATG |
| | AGATTCAAAATGGGAATGCGTCGCTATTTTTCAGAAGAGTAAGCCTTCTGGACG |
| | AAGGAATTTACACCTGCTATGTAGGAACAGCAATTCAAGTGATTACAAACAAA |
| | GTGGTGCTAAAGGTGGGAGTTTTTCTCACACCCGTGATGAAGTATGAAAAGAG |
| | GAACACAAACAGCTTCTTAATATGCAGCGTGTTAAGTGTTTATCCTCGTCCAAT |
| | TATCACGTGGAAAATGGACAACACACCTATCTCTGAAAACAACATGGAAGAAA |
| | CAGGGTCTTTGGATTCTTTTTCTATTAACAGCCCACTGAATATTACAGGATCAAA |
| | TTCATCTTATGAATGTACAATTGAAAATTCACTGCTGAAGCAAACATGGACAGG |
| | GCGCTGGACGATGAAAGATGGCCTTCATAAAATGCAAAGTGAACACGTTTCAC |
| | TCTCATGTCAACCTGTAAATGATTATTTTTCACCAAACCAAGACTTCAAAGTTAC |
| | TTGGTCCAGAATGAAAAGTGGGACTTTCTCTGTCCTGGCTTACTATCTGAGCTCC |
| | TCACAAAATACAATTATCAATGAATCCCGATTCTCATGGAACAAAGAGCTGATA |
| | AACCAGAGTGACTTCTCTATGAATTTGATGGATCTTAATCTTTCAGACAGTGGG |
| | GAATATTTATGCAATATTTCTTCGGATGAATATACTTTACTTACCATCCACACAG |
| | TGCATGTAGAACCGAGCCAAGAAACAGCTTCCCATAACAAAGGCTTATGGATTT |
| | TGGTGCCCTCTGCGATTTTGGCAGCTTTTCTGCTGATTTGGAGCGTAAAATGTTG |
| | CAGAGCCCAGCTAGAAGCCAGGAGGAGCAGACACCCTGCTGATGGAGCCCAAC |
| | AAGAAAGATGTTGTGTCCCTCCTGGTGAGCGCTGTCCCAGTGCACCCGATAATG |
| | GCGAAGAAAATGTGCCTCTTTCAGGAAAAGTATAGGAAATGAGAGAAGACTGT |
| | GACAACTCATGACCTGCATCCTTAATATCCAGTGACTTCATCTCCCCTTTCTTCA |
| | CCACAATTCCAGGCAATGGCCTGTCGGAGCAGACAATTCTACCACTGCAAAGA |
| | GTTGTAACCATTTTCTGGTATCACATTTATTTTTCAAGACATACTTTTCAAGACA |
| | TCATTCACTGACCCACTACCTGCATTGAGTATAAATGCCTGGATGTTAAGGATT |
| | CCAATTTAACTTTGAAAAGAACTGTCTCATTCATTTACATTTCTGTTACAGTCAG |
| | CCCAGGAGGTTACAGTGAGCTCTCCACTAAGAATCTGGAAGAAATGCATCACT |
| | AGGGGTTGATTCCCAATCTGATCAACTGATAATGGGTGAGAGAGCAGGTAAGA |
| | GCCAAAGTCACCTTAGTGGAAAGGTTAAAAACCAGAGCCTGGAAACCAAGATG |
| | ATTGATTTGACAAGGTATTTTAGTCTAGTTTTATATGAACGGTTGTATCAGGGTA |
| | ACCAACTCGATTTGGGATGAATCTTAGGGCACCAAAGACTAAGACAGTATCTTT |
| | AAGATTGCTAGGGAAAAGGGCCCTATGTGTCAGGCCTCTGAGCCCAAGCCAAG |
| | CATCGCATCCCCTGTGATTTGCACGTATACATCCAGATGGCCTAAAGTAACTGA |
| | AGATCCACAAAAGAAGTAAAAATAGCCTTAACTGATGACATTCCACCATTGTG |
| | ATTTGTTCCTGCCCCACCCTAACTGATCAATGTACTTTGTAATCTCCCCCACCCT |
| | TAAGAAGGTACTTTGTAATCTTCCCCACCCTTAAGAAGGTTCTTTGTAATTCTCC |
| | CCACCCTTGAGAATGTACTTTGTGAGATCCACCCTGCCCACAAAACATTGCTCT |
| | TAACTTCACCGCCTAACCCAAAACCTATAAGAACTAATGATAATCCATCACCCT |
| | TCGCTGACTCTCTTTTCGGACTCAGCCCACCTGCACCCAGGTGAAATAAACAGC |
| | TTTATTGCTCACACAAA (SEQ ID NO: 51) |
| | >NP_009003.1 HERV-H LTR-associating protein 2 isoform a |
| | precursor [Homo sapiens], amino acid sequence |
| | MKAQTALSFFLILITSLSGSQGIFPLAFFIYVPMNEQIVIGRLDEDIILPSSFERGSEVVI |
| | HWKYQDSYKVHSYYKGSDHLESQDPRYANRTSLFYNEIQNGNASLFFRRVSLLDE |
| | GIYTCYVGTAIQVITNKVVLKVGVFLTPVMKYEKRNTNSFLICSVLSVYPRPIITWK |
| | MDNTPISENNMEETGSLDSFSINSPLNITGSNSSYECTIENSLLKQTWTGRWTMKDG |
| | LHKMQSEHVSLSCQPVNDYFSPNQDFKVTWSRMKSGTFSVLAYYLSSSQNTIINES |
| | RFSWNKELINQSDFSMNLMDLNLSDSGEYLCNISSDEYTLLTIHTVHVEPSQETASH |
| | NKGLWILVPSAILAAFLLIWSVKCCRAQLEARRSRHPADGAQQERCCVPPGERCPS |
| | APDNGEENVPLSGKV (SEQ ID NO: 52) |
| Human | >NM_001013661.1 Homo sapiens V-set and immunoglobulin |
| VSIG8 | domain containing 8 (VSIG8), mRNA, nucleic acid sequence |
| | ACTCATTGCACCTTCCTGCCACCCCAGGCAGTGTCTGGGCCCTCAGCTCCCCCTC |
| | CCTCCACCTACCCCCTCACACCCACCACTACGACCCCACGGGATACCCAGCCCA |
| | GACGGAGGAAACACCGAGCCTAGAGACATGAGAGTTGGAGGAGCATTCCACCT |
| | TCTACTCGTGTGCCTGAGCCCAGCACTGCTGTCTGCTGTGCGGATCAACGGGGA |
| | TGGACAGGAGGTCCTGTACCTGGCAGAAGGTGATAATGTGAGGCTGGGCTGCC |
| | CCTACGTCCTGGACCCTGAGGACTATGGTCCCAATGGGCTGGACATCGAGTGGA |
| | TGCAGGTCAACTCAGACCCCGCCCACCACCGAGAGAACGTGTTCCTTAGTTACC |
| | AGGACAAGAGGATCAACCATGGCAGCCTTCCCCATCTGCAGCAGAGGGTCCGC |
| | TTTGCAGCCTCAGACCCAAGCCAGTACGATGCCTCCATCAACCTCATGAACCTG |
| | CAGGTATCTGATACAGCCACTTATGAGTGCCGGGTGAAGAAGACCACCATGGC |
| | CACCCGGAAGGTCATTGTCACTGTCCAAGCACGACCTGCAGTGCCCATGTGCTG |
| | GACAGAGGGCCACATGACATATGGCAACGATGTGGTGCTGAAGTGCTATGCCA |
| | GTGGGGGCTCCCAGCCCCTCTCCTACAAGTGGGCCAAGATCAGTGGGCACCATT |
| | ACCCCTATCGAGCTGGGTCTTACACCTCCCAGCACAGCTACCACTCAGAGCTGT |
| | CCTACCAGGAGTCCTTCCACAGCTCCATAAACCAAGGCCTGAACAATGGGGAC |
| | CTGGTGTTGAAGGATATCTCCAGAGCAGATGATGGGCTGTATCAGTGCACAGTG |
| | GCCAACAACGTGGGCTACAGTGTTTGTGTGGTGGAGGTGAAGGTCTCAGACTCC |
| | CGGCGTATAGGCGTGATCATCGGCATCGTCCTGGGCTCTCTGCTCGCGCTGGGC |
| | TGCCTGGCCGTAGGCATCTGGGGGCTCGTCTGCTGCTGCTGCGGGGGCTCCGGG |
| | GCTGGCGGCGCCCGCGGTGCCTTCGGCTACGGCAACGGCGGCGGGGTCGGCGG |
| | AGGGGCCTGCGGCGACTTGGCTAGTGAGATCAGAGAGGACGCCGTGGCGCCCG |
| | GGTGCAAGGCCAGCGGGCGCGGCAGCCGCGTCACCCACCTCCTGGGGTACCCG |
| | ACGCAGAACGTCAGCCGCTCCCTGCGCCGCAAGTACGCGCCTCCCCCCTGCGGC |
| | GGCCCCGAGGACGTGGCCCTGGCGCCCTGCACCGCCGCCGCCGCCTGCGAAGC |
| | GGGCCCCTCCCCGGTCTACGTCAAGGTCAAGAGCGCGGAGCCGGCTGACTGCG |
| | CCGAGGGGCCGGTGCAGTGCAAGAACGGCCTCTTGGTGTGAGCGCGCGCGCCG |
| | GGCCGGGCTGCGCCCCAGCCAGGAGGAGGGCGCGGGGCTCTCTGTCTGCAGCT |
| | GGGGACACGTCGGGGCTGGGGACGACCTCGCTCGCCCCAGGCTGCCAGGCGGC |
| | TGGGGGTGAAGGCATTTCCCTAAGGAAATGCGTAGGGAGGCAGAGCCTCCTCC |
| | CCAAAAGTGGGAAGGGGCGGGCGAGGGCGGAGGAAGGCGATCCTGAGCCTTCT |
| | CCGCACCCCCGGGACCGAAGGCTTGGGGGAGAGGGAGGGAGGAGGAGGCTGA |
| | GTGTCCTAGAGCGGCTGAGGCCGGAGGCCTGGTGTCCCCAGCCTAAGCAGAGG |
| | GCCCCGGGGGCCGGGTGGGTGGGGGTCTGTCTGGACGAATTGTTCTGTGTGTGA |
| | GGTCTGAGCTCTGAGGCAGCAGTGTTAGCACAATAAAGAAACATTGAGACGTG |
| | A (SEQ ID NO: 53) |
| | >NP_001013683.1 V-set and immunoglobulin domain- |
| | containing protein 8 precursor [Homo sapiens], amino |
| | acid sequence |
| | MRVGGAFHLLLVCLSPALLSAVRINGDGQEVLYLAEGDNVRLGCPYVLDPEDYGP |
| | NGLDIEWMQVNSDPAHHRENVFLSYQDKRINHGSLPHLQQRVRFAASDPSQYDASI |
| | NLMNLQVSDTATYECRVKKTTMATRKVIVTVQARPAVPMCWTEGHMTYGNDVV |
| | LKCYASGGSQPLSYKWAKISGHHYPYRAGSYTSQHSYHSELSYQESFHSSINQGLN |
| | NGDLVLKDISRADDGLYQCTVANNVGYSVCVVEVKVSDSRRIGVIIGIVLGSLLAL |
| | GCLAVGIWGLVCCCCGGSGAGGARGAFGYGNGGGVGGGACGDLASEIREDAVAP |
| | GCKASGRGSRVTHLLGYPTQNVSRSLRRKYAPPPCGGPEDVALAPCTAAAACEAG |
| | PSPVYVKVKSAEPADCAEGPVQCKNGLLV (SEQ ID NO: 54) |
| Human | >NM_001015887.3 Homo sapiens immunoglobulin superfamily |
| VSIG3 | member 11 (IGSF11), transcript variant 2, mRNA, nucleic |
| (IGSF11) | acid sequence |
| | AGTCCTGGGGCAGGGCTGGGTGGCACGGCTGGCGAGCCCGGAACGCCTCTGGT |
| | CACAGCTCAGCGTCCGCGGAGCCGGGCGGCGCTGCAGCTGCACTTGGCTCGTCT |
| | GTGGGTCTGACAGTCCCAGCTCTGCGCGGGGAACAGCGGCCCGGCGCTGGGTG |
| | TGGGAGGACCAGGCTGCCCCAAGAGCGCGGAGACTCACGCCCGCTCCTCTCCT |
| | GTTGCGACCGGGAGCCGGGTAGGAGGCAGGCGCGCTCCCTGCGGCCCCGGGAT |
| | GACTTCTCAGCGTTCCCCTCTGGCGCCTTTGCTGCTCCTCTCTCTGCACGGTGTT |
| | GCAGCATCCCTGGAAGTGTCAGAGAGCCCTGGGAGTATCCAGGTGGCCCGGGG |
| | TCAGCCAGCAGTCCTGCCCTGCACTTTCACTACCAGCGCTGCCCTCATTAACCTC |
| | AATGTCATTTGGATGGTCACTCCTCTCTCCAATGCCAACCAACCTGAACAGGTC |
| | ATCCTGTATCAGGGTGGACAGATGTTTGATGGTGCCCCCCGGTTCCACGGTAGG |
| | GTAGGATTTACAGGCACCATGCCAGCTACCAATGTCTCTATCTTCATTAATAAC |
| | ACTCAGTTATCAGACACTGGCACCTACCAGTGCCTGGTCAACAACCTTCCAGAC |
| | ATAGGGGGCAGGAACATTGGGGTCACCGGTCTCACAGTGTTAGTTCCCCCTTCT |
| | GCCCCACACTGCCAAATCCAAGGATCCCAGGATATTGGCAGCGATGTCATCCTG |
| | CTCTGTAGCTCAGAGGAAGGCATTCCTCGACCAACTTACCTTTGGGAGAAGTTA |
| | GACAATACCCTCAAACTACCTCCAACAGCTACTCAGGACCAGGTCCAGGGAAC |
| | AGTCACCATCCGGAACATCAGTGCCCTGTCTTCAGGTTTGTACCAGTGCGTGGC |
| | TTCTAATGCTATTGGAACCAGCACCTGTCTTCTGGATCTCCAGGTTATTTCACCC |
| | CAGCCCAGGAACATTGGACTAATAGCTGGAGCCATTGGCACTGGTGCAGTTATT |
| | ATCATTTTTTGCATTGCACTAATTTTAGGGGCATTCTTTTACTGGAGAAGCAAAA |
| | ATAAAGAGGAGGAAGAAGAAGAAATTCCTAATGAAATAAGAGAGGATGATCTT |
| | CCACCCAAGTGTTCTTCTGCCAAAGCATTTCACACTGAGATTTCCTCCTCGGAC |
| | AACAACACACTAACCTCTTCCAATGCCTACAACAGTCGATACTGGAGCAACAAT |
| | CCAAAAGTTCATAGAAACACAGAGTCAGTCAGCCACTTCAGTGACTTGGGCCA |
| | ATCTTTCTCTTTCCACTCAGGCAATGCCAACATACCATCCATTTATGCTAATGGG |
| | ACCCATCTGGTCCCGGGTCAACATAAGACTCTGGTAGTGACAGCCAACAGAGG |
| | GTCATCACCACAGGTGATGTCCAGGAGCAATGGCTCAGTCAGTAGGAAGCCTC |
| | GGCCTCCACACACTCATTCCTACACCATCAGCCACGCAACACTGGAACGAATTG |
| | GTGCAGTACCTGTCATGGTACCAGCCCAGAGTCGGGCCGGGTCCTTGGTATAGG |
| | ACATGAGGAAATGTTGTGTTCAGAAATGAATAAATGGAATGCCCTCATACAAG |
| | GGGGAGGGTGGGGTGGGGAGTGCTGGGAAAGAAACACTTCCTTATAATTATAT |
| | TAGTAAAATGCACAAAGAAGAAGGCAGTGCTGTTACTTGGCCACTAAGATGTG |
| | TAAAATGGACTGAAATGCTCCATCATGAAGACTTGCTTCCCCACCAAAGATGTC |
| | CTGGGATTCTGCTGGATCTCAAAGATGTGCCAAGCCAAGGAAAAAGATACAAG |
| | AGCAGAATAGTACTTAAAATCCAAACTGCCGCCCAGATGGGCTTGTTCTTCATG |
| | CCTAACTTAATAATTTTTAAGAGATTAAAGTGCCAGATGGAGTTTAAATATTGA |
| | AATTATTTTAAAAGGTAGGTGTCTTTAAGAAAATAACAAGCAACCCTGTGATAT |
| | GTTCCGTCTCTCCCAATTCCCTCGTTATATAGAGGGCTTAATGGTATAAATGGTT |
| | AATATTGGTCCCAACAGGGCTGACTCTTCTATCATATAATCAAAACTTTTTACAT |
| | GAGCAAAATTCAGTAAGAAATGGGGGAAGACAAAGGAAACGTCTTTGAGAAG |
| | CCCCTTCATATTTATTTATTTATCTCTTCCTGAACCATGAATTTCATATGTGGAA |
| | TATTGCTATATTGACAGATTCTTGCCTGTCTGTGTTATTCTAGGATCTGTTACAG |
| | GTCCATGGCAATTACTGTTTATTTTTTCCTGGAAAAATATTTTTTTATAAAAGGC |
| | TTTTTTTTTTTTTTAAATACATGAGAGGCATTGGGCTAAGAAAGAAAAGACTGT |
| | TGTATAATACCTTGTTCAATGGTTGTATTTAGTGAGCTCATAGAGGTCCATCATA |
| | TCATGACCGAGCTAGGTTGTGTGGGCAGGAAGGTAGGGCTAAGGGGTTGTAGC |
| | CTTGCTGGGCAGCCTCTCAGAGCAAGGTTGTTCAGATCTCCCTTGCTATTACAGT |
| | AGGTTACTATTAATGAGGGCAGCACCTGATGCCTTTTGTACTGAGGTATGTAAC |
| | TTTCTCCTTATTTGACAAGTAGAAGTTAACTTACTTGTCAGGGAGGGCAGACGT |
| | TTTTTTGTTCTGTTTCGTTTTTCAAAATAATGCTTTTTGCAAAAGAGGTAAGACT |
| | GAGACTAAAGGTGTTATCTTCTGGTGTGCTCCTGGAAGTGTCTACCCTACATTTG |
| | TGTCAGCTCAGGGTTGCAGTGTTGCCCAGATGCATTTTACATCACTGTAAAGAG |
| | ATTACTTTTGTGGTTACTACCTGGCTTGGCTGGCCTTGCGGTTCACCAGATTAAT |
| | TTACAAACTCCCCCACTTTATTTTGTGCTATGTAGATCTGGCCATACTTGCATTA |
| | GTGACTGTCTTGCCTTAACCACACTTAAGCAACCCACAAATTTCTTCTCAGATTT |
| | GTTTCCTAGATTACTTATGATACTCATCCCATGTCTCAATAAGAGTGTCTTTTCT |
| | TTCTGGATGTGTTCTCTTACTCCCTCTTACCACCATACTTTTTGCTCTCTTCTCCT |
| | GCAAGCGTAGTCTTCACAGGGAGTGGCTTCCTGACATTTTTTTCAGTTATGTGA |
| | ATGAATGGAAACCAACAGCTGCTGCAAACACTGTTTTTCCAAGAAGGCTACACT |
| | CAGAACCTAACCATTGCCAACCATTTCAGTATTGATAAAAAGCTGAATTTACTT |
| | TAGCATTACTTATTTTTTTTTCCATTTGATGGTTCTTACTTTGTAAAAATTTAAAT |
| | AAATGAATGTCTATACTTTTTATAAAGAAAAGTGAAAATACCATGACACTGAAA |
| | AGATGATGCTATCAGATGCTGTTTAGAAAGCATTTATCTTGCATTTCTTTATTCT |
| | TTCTAATTATCTAAAATTCAATAAAATTTTATTCATATAAAATAAGTIGTCATTA |
| | ATTATCAATACTAACGAGTATGTCATTTTAAAACTTAGTATTCTCTTTAATGTTA |
| | CAAGA (SEQ ID NO: 55) |
| | >NP_001015887.1 immunoglobulin superfamily member 11 |
| | isoform b precursor [Homo sapiens], amino acid sequence |
| | MTSQRSPLAPLLLLSLHGVAASLEVSESPGSIQVARGQPAVLPCTFTTSAALINLNVI |
| | WMVTPLSNANQPEQVILYQGGQMFDGAPRFHGRVGFTGTMPATNVSIFINNTQLS |
| | DTGTYQCLVNNLPDIGGRNIGVTGLTVLVPPSAPHCQIQGSQDIGSDVILLCSSEEGI |
| | PRPTYLWEKLDNTLKLPPTATQDQVQGTVTIRNISALSSGLYQCVASNAIGTSTCLL |
| | DLQVISPQPRNIGLIAGAIGTGAVIIIFCIALILGAFFYWRSKNKEEEEEEIPNEIREDDL |
| | PPKCSSAKAFHTEISSSDNNTLTSSNAYNSRYWSNNPKVHRNTESVSHFSDLGQSFS |
| | FHSGNANIPSIYANGTHLVPGQHKTLVVTANRGSSPQVMSRSNGSVSRKPRPPHTH |
| | SYTISHATLERIGAVPVMVPAQSRAGSLV |
| | (SEQ ID NO: 56) |
| Human | >NM_007268.3 Homo sapiens V-set and immunoglobulin domain |
| VSIG4 | containing 4 (VSIG4), transcript variant 1, mRNA, nucleic |
| | acid sequence |
| | ACAGACGCTGGCGGCCACCAGAAGTTTGAGCCTCTTTGGTAGCAGGAGGCTGG |
| | AAGAAAGGACAGAAGTAGCTCTGGCTGTGATGGGGATCTTACTGGGCCTGCTA |
| | CTCCTGGGGCACCTAACAGTGGACACTTATGGCCGTCCCATCCTGGAAGTGCCA |
| | GAGAGTGTAACAGGACCTTGGAAAGGGGATGTGAATCTTCCCTGCACCTATGA |
| | CCCCCTGCAAGGCTACACCCAAGTCTTGGTGAAGTGGCTGGTACAACGTGGCTC |
| | AGACCCTGTCACCATCTTTCTACGTGACTCTTCTGGAGACCATATCCAGCAGGC |
| | AAAGTACCAGGGCCGCCTGCATGTGAGCCACAAGGTTCCAGGAGATGTATCCC |
| | TCCAATTGAGCACCCTGGAGATGGATGACCGGAGCCACTACACGTGTGAAGTC |
| | ACCTGGCAGACTCCTGATGGCAACCAAGTCGTGAGAGATAAGATTACTGAGCT |
| | CCGTGTCCAGAAACTCTCTGTCTCCAAGCCCACAGTGACAACTGGCAGCGGTTA |
| | TGGCTTCACGGTGCCCCAGGGAATGAGGATTAGCCTTCAATGCCAGGCTCGGGG |
| | TTCTCCTCCCATCAGTTATATTTGGTATAAGCAACAGACTAATAACCAGGAACC |
| | CATCAAAGTAGCAACCCTAAGTACCTTACTCTTCAAGCCTGCGGTGATAGCCGA |
| | CTCAGGCTCCTATTTCTGCACTGCCAAGGGCCAGGTTGGCTCTGAGCAGCACAG |
| | CGACATTGTGAAGTTTGTGGTCAAAGACTCCTCAAAGCTACTCAAGACCAAGAC |
| | TGAGGCACCTACAACCATGACATACCCCTTGAAAGCAACATCTACAGTGAAGC |
| | AGTCCTGGGACTGGACCACTGACATGGATGGCTACCTTGGAGAGACCAGTGCT |
| | GGGCCAGGAAAGAGCCTGCCTGTCTTTGCCATCATCCTCATCATCTCCTTGTGCT |
| | GTATGGTGGTTTTTACCATGGCCTATATCATGCTCTGTCGGAAGACATCCCAAC |
| | AAGAGCATGTCTACGAAGCAGCCAGGGCACATGCCAGAGAGGCCAACGACTCT |
| | GGAGAAACCATGAGGGTGGCCATCTTCGCAAGTGGCTGCTCCAGTGATGAGCC |
| | AACTTCCCAGAATCTGGGCAACAACTACTCTGATGAGCCCTGCATAGGACAGG |
| | AGTACCAGATCATCGCCCAGATCAATGGCAACTACGCCCGCCTGCTGGACACA |
| | GTTCCTCTGGATTATGAGTTTCTGGCCACTGAGGGCAAAAGTGTCTGTTAAAAA |
| | TGCCCCATTAGGCCAGGATCTGCTGACATAATTGCCTAGTCAGTCCTTGCCTTCT |
| | GCATGGCCTTCTTCCCTGCTACCTCTCTTCCTGGATAGCCCAAAGTGTCCGCCTA |
| | CCAACACTGGAGCCGCTGGGAGTCACTGGCTTTGCCCTGGAATTTGCCAGATGC |
| | ATCTCAAGTAAGCCAGCTGCTGGATTTGGCTCTGGGCCCTTCTAGTATCTCTGCC |
| | GGGGGCTTCTGGTACTCCTCTCTAAATACCAGAGGGAAGATGCCCATAGCACTA |
| | GGACTTGGTCATCATGCCTACAGACACTATTCAACTTTGGCATCTTGCCACCAG |
| | AAGACCCGAGGGAGGCTCAGCTCTGCCAGCTCAGAGGACCAGCTATATCCAGG |
| | ATCATTTCTCTTTCTTCAGGGCCAGACAGCTTTTAATTGAAATTGTTATTTCACA |
| | GGCCAGGGTTCAGTTCTGCTCCTCCACTATAAGTCTAATGTTCTGACTCTCTCCT |
| | GGTGCTCAATAAATATCTAATCATAACAGCAA (SEQ ID NO: 57) |
| | >NP_009199.1 V-set and immunoglobulin domain-containing |
| | protein 4 isoform 1 precursor [Homo sapiens], amino |
| | acid sequence |
| | MGILLGLLLLGHLTVDTYGRPILEVPESVTGPWKGDVNLPCTYDPLQGYTQVLVK |
| | WLVQRGSDPVTIFLRDSSGDHIQQAKYQGRLHVSHKVPGDVSLQLSTLEMDDRSH |
| | YTCEVTWQTPDGNQVVRDKITELRVQKLSVSKPTVTTGSGYGFTVPQGMRISLQC |
| | QARGSPPISYIWYKQQTNNQEPIKVATLSTLLFKPAVIADSGSYFCTAKGQVGSEQH |
| | SDIVKFVVKDSSKLLKTKTEAPTTMTYPLKATSTVKQSWDWTTDMDGYLGETSAG |
| | PGKSLPVFAIILIISLCCMVVFTMAYIMLCRKTSQQEHVYEAARAHAREANDSGET |
| | MRVAIFASGCSSDEPTSQNLGNNYSDEPCIGQEYQIIAQINGNYARLLDTVPLDYEF |
| | LATEGKSVC (SEQ ID NO: 58) |
| Human | >NM_032782.5 Homo sapiens hepatitis A virus cellular |
| Tim-3 | receptor 2 (HAVCR2), mRNA, nucleic acid sequence |
| (HAVCR2) | ATTTGGAGAGTTAAAACTGTGCCTAACAGAGGTGTCCTCTGACTTTTCTTCTGCA |
| | AGCTCCATGTTTTCACATCTTCCCTTTGACTGTGTCCTGCTGCTGCTGCTGCTAC |
| | TACTTACAAGGTCCTCAGAAGTGGAATACAGAGCGGAGGTCGGTCAGAATGCC |
| | TATCTGCCCTGCTTCTACACCCCAGCCGCCCCAGGGAACCTCGTGCCCGTCTGC |
| | TGGGGCAAAGGAGCCTGTCCTGTGTTTGAATGTGGCAACGTGGTGCTCAGGACT |
| | GATGAAAGGGATGTGAATTATTGGACATCCAGATACTGGCTAAATGGGGATTTC |
| | CGCAAAGGAGATGTGTCCCTGACCATAGAGAATGTGACTCTAGCAGACAGTGG |
| | GATCTACTGCTGCCGGATCCAAATCCCAGGCATAATGAATGATGAAAAATTTAA |
| | CCTGAAGTTGGTCATCAAACCAGCCAAGGTCACCCCTGCACCGACTCGGCAGA |
| | GAGACTTCACTGCAGCCTTTCCAAGGATGCTTACCACCAGGGGACATGGCCCAG |
| | CAGAGACACAGACACTGGGGAGCCTCCCTGATATAAATCTAACACAAATATCC |
| | ACATTGGCCAATGAGTTACGGGACTCTAGATTGGCCAATGACTTACGGGACTCT |
| | GGAGCAACCATCAGAATAGGCATCTACATCGGAGCAGGGATCTGTGCTGGGCT |
| | GGCTCTGGCTCTTATCTTCGGCGCTTTAATTTTCAAATGGTATTCTCATAGCAAA |
| | GAGAAGATACAGAATTTAAGCCTCATCTCTTTGGCCAACCTCCCTCCCTCAGGA |
| | TTGGCAAATGCAGTAGCAGAGGGAATTCGCTCAGAAGAAAACATCTATACCAT |
| | TGAAGAGAACGTATATGAAGTGGAGGAGCCCAATGAGTATTATTGCTATGTCA |
| | GCAGCAGGCAGCAACCCTCACAACCTTTGGGTTGTCGCTTTGCAATGCCATAGA |
| | TCCAACCACCTTATTTTTGAGCTTGGTGTTTTGTCTTTTTCAGAAACTATGAGCT |
| | GTGTCACCTGACTGGTTTTGGAGGTTCTGTCCACTGCTATGGAGCAGAGTTTTCC |
| | CATTTTCAGAAGATAATGACTCACATGGGAATTGAACTGGGACCTGCACTGAAC |
| | TTAAACAGGCATGTCATTGCCTCTGTATTTAAGCCAACAGAGTTACCCAACCCA |
| | GAGACTGTTAATCATGGATGTTAGAGCTCAAACGGGCTTTTATATACACTAGGA |
| | ATTCTTGACGTGGGGTCTCTGGAGCTCCAGGAAATTCGGGCACATCATATGTCC |
| | ATGAAACTTCAGATAAACTAGGGAAAACTGGGTGCTGAGGTGAAAGCATAACT |
| | TTTTTGGCACAGAAAGTCTAAAGGGGCCACTGATTTTCAAAGAGATCTGTGATC |
| | CCTTTTTGTTTTTTGTTTTTGAGATGGAGTCTTGCTCTGTTGCCCAGGCTGGAGT |
| | GCAATGGCACAATCTCGGCTCACTGCAAGCTCCGCCTCCTGGGTTCAAGCGATT |
| | CTCCTGCCTCAGCCTCCTGAGTGGCTGGGATTACAGGCATGCACCACCATGCCC |
| | AGCTAATTTGTTGTATTTTTAGTAGAGACAGGGTTTCACCATGTTGGCCAGTGTG |
| | GTCTCAAACTCCTGACCTCATGATTTGCCTGCCTCGGCCTCCCAAAGCACTGGG |
| | ATTACAGGCGTGAGCCACCACATCCAGCCAGTGATCCTTAAAAGATTAAGAGA |
| | TGACTGGACCAGGTCTACCTTGATCTTGAAGATTCCCTTGGAATGTTGAGATTT |
| | AGGCTTATTTGAGCACTGCCTGCCCAACTGTCAGTGCCAGTGCATAGCCCTTCT |
| | TTTGTCTCCCTTATGAAGACTGCCCTGCAGGGCTGAGATGTGGCAGGAGCTCCC |
| | AGGGAAAAACGAAGTGCATTTGATTGGTGTGTATTGGCCAAGTTTTGCTTGTTG |
| | TGTGCTTGAAAGAAAATATCTCTGACCAACTTCTGTATTCGTGGACCAAACTGA |
| | AGCTATATTTTTCACAGAAGAAGAAGCAGTGACGGGGACACAAATTCTGTTGCC |
| | TGGTGGAAAGAAGGCAAAGGCCTTCAGCAATCTATATTACCAGCGCTGGATCCT |
| | TTGACAGAGAGTGGTCCCTAAACTTAAATTTCAAGACGGTATAGGCTTGATCTG |
| | TCTTGCTTATTGTTGCCCCCTGCGCCTAGCACAATTCTGACACACAATTGGAACT |
| | TACTAAAAATTTTTTTTTACTGTT |
| | (SEQ ID NO: 59) |
| | >NP_116171.3 hepatitis A virus cellular receptor 2 |
| | precursor [Homo sapiens], amino acid sequence |
| | MFSHLPFDCVLLLLLLLLTRSSEVEYRAEVGQNAYLPCFYTPAAPGNLVPVCWGKG |
| | ACPVFECGNVVLRTDERDVNYWTSRYWLNGDFRKGDVSLTIENVTLADSGIYCCR |
| | IQIPGIMNDEKFNLKLVIKPAKVTPAPTRQRDFTAAFPRMLTTRGHGPAETQTLGSL |
| | PDINLTQISTLANELRDSRLANDLRDSGATIRIGIYIGAGICAGLALALIFGALIFKWY |
| | SHSKEKIQNLSLISLANLPPSGLANAVAEGIRSEENIYTIEENVYEVEEPNEYYCYVSS |
| | RQQPSQPLGCRFAMP (SEQ ID NO: 60) |
| Human | >NM_138379.3 Homo sapiens T cell immunoglobulin and mucin |
| Tim-4 | domain containing 4 (TIMD4), transcript variant 1, mRNA, |
| (TIMD4) | nucleic acid sequence |
| | AGACTCCTGGGTCCGGTCAACCGTCAAAATGTCCAAAGAACCTCTCATTCTCTG |
| | GCTGATGATTGAGTTTTGGTGGCTTTACCTGACACCAGTCACTTCAGAGACTGTT |
| | GTGACGGAGGTTTTGGGTCACCGGGTGACTTTGCCCTGTCTGTACTCATCCTGGT |
| | CTCACAACAGCAACAGCATGTGCTGGGGGAAAGACCAGTGCCCCTACTCCGGT |
| | TGCAAGGAGGCGCTCATCCGCACTGATGGAATGAGGGTGACCTCAAGAAAGTC |
| | AGCAAAATATAGACTTCAGGGGACTATCCCGAGAGGTGATGTCTCCTTGACCAT |
| | CTTAAACCCCAGTGAAAGTGACAGCGGTGTGTACTGCTGCCGCATAGAAGTGCC |
| | TGGCTGGTTCAACGATGTAAAGATAAACGTGCGCCTGAATCTACAGAGAGCCTC |
| | AACAACCACGCACAGAACAGCAACCACCACCACACGCAGAACAACAACAACA |
| | AGCCCCACCACCACCCGACAAATGACAACAACCCCAGCTGCACTTCCAACAAC |
| | AGTCGTGACCACACCCGATCTCACAACCGGAACACCACTCCAGATGACAACCA |
| | TTGCCGTCTTCACAACAGCAAACACGTGCCTTTCACTAACCCCAAGCACCCTTC |
| | CGGAGGAAGCCACAGGTCTTCTGACTCCCGAGCCTTCTAAGGAAGGGCCCATCC |
| | TCACTGCAGAATCAGAAACTGTCCTCCCCAGTGATTCCTGGAGTAGTGTTGAGT |
| | CTACTTCTGCTGACACTGTCCTGCTGACATCCAAAGAGTCCAAAGTTTGGGATC |
| | TCCCATCAACATCCCACGTGTCAATGTGGAAAACGAGTGATTCTGTGTCTTCTC |
| | CTCAGCCTGGAGCATCTGATACAGCAGTTCCTGAGCAGAACAAAACAACAAAA |
| | ACAGGACAGATGGATGGAATACCCATGTCAATGAAGAATGAAATGCCCATCTC |
| | CCAACTACTGATGATCATCGCCCCCTCCTTGGGATTTGTGCTCTTCGCATTGTTT |
| | GTGGCGTTTCTCCTGAGAGGGAAACTCATGGAAACCTATTGTTCGCAGAAACAC |
| | ACAAGGCTAGACTACATTGGAGATAGTAAAAATGTCCTCAATGACGTGCAGCA |
| | TGGAAGGGAAGACGAAGACGGCCTTTTTACCCTCTAACAACGCAGTAGCATGTT |
| | AGATTGAGGATGGGGGCATGACACTCCAGTGTCAAAATAAGTCTTAGTAGATTT |
| | CCTTGTTTCATAAAAAAGACTCACTTATTCCATGGATGTCATTGATCCAGGCTTG |
| | CTTTAGTTTCATGAATGAAGGGTACTTTAGAGACCACAA (SEQ ID NO: 61) |
| | >NP_612388.2 T-cell immunoglobulin and mucin domain- |
| | containing protein 4 isoform 1 precursor [Homo sapiens], |
| | amino acid sequence |
| | MSKEPLILWLMIEFWWLYLTPVTSETVVTEVLGHRVTLPCLYSSWSHNSNSMCWG |
| | KDQCPYSGCKEALIRTDGMRVTSRKSAKYRLQGTIPRGDVSLTILNPSESDSGVYCC |
| | RIEVPGWFNDVKINVRLNLQRASTTTHRTATTTTRRTTTTSPTTTRQMTTTPAALPT |
| | TVVTTPDLTTGTPLQMTTIAVFTTANTCLSLTPSTLPEEATGLLTPEPSKEGPILTAES |
| | ETVLPSDSWSSVESTSADTVLLTSKESKVWDLPSTSHVSMWKTSDSVSSPQPGASD |
| | TAVPEQNKTTKTGQMDGIPMSMKNEMPISQLLMIIAPSLGFVLFALFVAFLLRGKL |
| | METYCSQKHTRLDYIGDSKNVLNDVQHGREDEDGLFTL (SEQ ID NO: 62) |
| Human | >NM_001712.5 Homo sapiens CEA cell adhesion molecule 1 |
| CEACAM1 | (CEACAM1), transcript variant 1, mRNA, nucleic acid sequence |
| | AGCACAGAGAGTGGAAAACAGCAGAGGTGACAGAGCAGCCGTGCTCGAAGCG |
| | TTCCTGGAGCCCAAGCTCTCCTCCACAGGTGAAGACAGGGCCAGCAGGAGACA |
| | CCATGGGGCACCTCTCAGCCCCACTTCACAGAGTGCGTGTACCCTGGCAGGGGC |
| | TTCTGCTCACAGCCTCACTTCTAACCTTCTGGAACCCGCCCACCACTGCCCAGCT |
| | CACTACTGAATCCATGCCATTCAATGTTGCAGAGGGGAAGGAGGTTCTTCTCCT |
| | TGTCCACAATCTGCCCCAGCAACTTTTTGGCTACAGCTGGTACAAAGGGGAAAG |
| | AGTGGATGGCAACCGTCAAATTGTAGGATATGCAATAGGAACTCAACAAGCTA |
| | CCCCAGGGCCCGCAAACAGCGGTCGAGAGACAATATACCCCAATGCATCCCTG |
| | CTGATCCAGAACGTCACCCAGAATGACACAGGATTCTACACCCTACAAGTCATA |
| | AAGTCAGATCTTGTGAATGAAGAAGCAACTGGACAGTTCCATGTATACCCGGA |
| | GCTGCCCAAGCCCTCCATCTCCAGCAACAACTCCAACCCTGTGGAGGACAAGG |
| | ATGCTGTGGCCTTCACCTGTGAACCTGAGACTCAGGACACAACCTACCTGTGGT |
| | GGATAAACAATCAGAGCCTCCCGGTCAGTCCCAGGCTGCAGCTGTCCAATGGC |
| | AACAGGACCCTCACTCTACTCAGTGTCACAAGGAATGACACAGGACCCTATGA |
| | GTGTGAAATACAGAACCCAGTGAGTGCGAACCGCAGTGACCCAGTCACCTTGA |
| | ATGTCACCTATGGCCCGGACACCCCCACCATTTCCCCTTCAGACACCTATTACC |
| | GTCCAGGGGCAAACCTCAGCCTCTCCTGCTATGCAGCCTCTAACCCACCTGCAC |
| | AGTACTCCTGGCTTATCAATGGAACATTCCAGCAAAGCACACAAGAGCTCTTTA |
| | TCCCTAACATCACTGTGAATAATAGTGGATCCTATACCTGCCACGCCAATAACT |
| | CAGTCACTGGCTGCAACAGGACCACAGTCAAGACGATCATAGTCACTGAGCTA |
| | AGTCCAGTAGTAGCAAAGCCCCAAATCAAAGCCAGCAAGACCACAGTCACAGG |
| | AGATAAGGACTCTGTGAACCTGACCTGCTCCACAAATGACACTGGAATCTCCAT |
| | CCGTTGGTTCTTCAAAAACCAGAGTCTCCCGTCCTCGGAGAGGATGAAGCTGTC |
| | CCAGGGCAACACCACCCTCAGCATAAACCCTGTCAAGAGGGAGGATGCTGGGA |
| | CGTATTGGTGTGAGGTCTTCAACCCAATCAGTAAGAACCAAAGCGACCCCATCA |
| | TGCTGAACGTAAACTATAATGCTCTACCACAAGAAAATGGCCTCTCACCTGGGG |
| | CCATTGCTGGCATTGTGATTGGAGTAGTGGCCCTGGTTGCTCTGATAGCAGTAG |
| | CCCTGGCATGTTTTCTGCATTTCGGGAAGACCGGCAGGGCAAGCGACCAGCGTG |
| | ATCTCACAGAGCACAAACCCTCAGTCTCCAACCACACTCAGGACCACTCCAATG |
| | ACCCACCTAACAAGATGAATGAAGTTACTTATTCTACCCTGAACTTTGAAGCCC |
| | AGCAACCCACACAACCAACTTCAGCCTCCCCATCCCTAACAGCCACAGAAATA |
| | ATTTATTCAGAAGTAAAAAAGCAGTAATGAAACCTGTCCTGCTCACTGCAGTGC |
| | TGATGTATTTCAAGTCTCTCACCCTCATCACTAGGAGATTCCTTTCCCCTGTAGG |
| | GGTAGAGGGGTGGGGACAGAAACAACTTTCTCCTACTCTTCCTTCCTAATAGGC |
| | ATCTCCAGGCTGCCTGGTCACTGCCCCTCTCTCAGTGTCAATAGATGAAAGTAC |
| | ATTGGGAGTCTGTAGGAAACCCAACCTTCTTGTCATTGAAATTTGGCAAAGCTG |
| | ACTTTGGGAAAGAGGGACCAGAACTTCCCCTCCCTTCCCCTTTTCCCAACCTGG |
| | ACTTGTTTTAAACTTGCCTGTTCAGAGCACTCATTCCTTCCCACCCCCAGTCCTG |
| | TCCTATCACTCTAATTCGGATTTGCCATAGCCTTGAGGTTATGTCCTTTTCCATT |
| | AAGTACATGTGCCAGGAAACAAGAGAGAGAGAAAGTAAAGGCAGTAATGCCTT |
| | CTCCTATTTCTCCAAAGCCTTGTGTGAACTCACCAAACACAAGAAAATCAAATA |
| | TATAACCAATAGTGAAATGCCACACCTTTGTCCACTGTCAGGGTTGTCTACCTG |
| | TAGGATCAGGGTCTAAGCACCTTGGTGCTTAGCTAGAATACCACCTAATCCTTC |
| | TGGCAAGCCTGTCTTCAGAGAACCCACTAGAAGCAACTAGGAAAATCACTTGC |
| | CAAAATCCAAGGCAATTCCTGATGGAAAATGCAAAAGCACATATATGTTTTAAT |
| | ATCTTTATGGGCTCTGTTCAAGGCAGTGCTGAGAGGGAGGGGTTATAGCTTCAG |
| | GAGGGAACCAGCTTCTGATAAACACAATCTGCTAGGAACTTGGGAAAGGAATC |
| | AGAGAGCTGCCCTTCAGCGATTATTTAAATTATTGTTAAAGAATACACAATTTG |
| | GGGTATTGGGATTTTTCTCCTTTTCTCTGAGACATTCCACCATTTTAATTTTTGTA |
| | ACTGCTTATTTATGTGAAAAGGGTTATTTTTACTTAGCTTAGCTATGTCAGCCAA |
| | TCCGATTGCCTTAGGTGAAAGAAACCACCGAAATCCCTCAGGTCCCTTGGTCAG |
| | GAGCCTCTCAAGATTTTTTTTGTCAGAGGCTCCAAATAGAAAATAAGAAAAGGT |
| | TTTCTTCATTCATGGCTAGAGCTAGATTTAACTCAGTTTCTAGGCACCTCAGACC |
| | AATCATCAACTACCATTCTATTCCATGTTTGCACCTGTGCATTTTCTGTTTGCCC |
| | CCATTCACTTTGTCAGGAAACCTTGGCCTCTGCTAAGGTGTATTTGGTCCTTGAG |
| | AAGTGGGAGCACCCTACAGGGACACTATCACTCATGCTGGTGGCATTGTTTACA |
| | GCTAGAAAGCTGCACTGGTGCTAATGCCCCTTGGGGAAATGGGGCTGTGAGGA |
| | GGAGGATTATAACTTAGGCCTAGCCTCTTTTAACAGCCTCTGAAATTTATCTTTT |
| | CTTCTATGGGGTCTATAAATGTATCTTATAATAAAAAGGAAGGACAGGAGGAA |
| | GACAGGCAAATGTACTTCTCACCCAGTCTTCTACACAGATGGAATCTCTTTGGG |
| | GCTAAGAGAAAGGTTTTATTCTATATTGCTTACCTGATCTCATGTTAGGCCTAAG |
| | AGGCTTTCTCCAGGAGGATTAGCTTGGAGTTCTCTATACTCAGGTACCTCTTTCA |
| | GGGTTTTCTAACCCTGACACGGACTGTGCATACTTTCCCTCATCCATGCTGTGCT |
| | GTGTTATTTAATTTTTCCTGGCTAAGATCATGTCTGAATTATGTATGAAAATTAT |
| | TCTATGTTTTTATAATAAAAATAATATATCAGACATCGA (SEQ ID NO: 63) |
| | >NP_001703.2 carcinoembryonic antigen-related cell adhesion |
| | molecule 1 isoform 1 precursor [Homo sapiens], amino acid |
| | sequence |
| | MGHLSAPLHRVRVPWQGLLLTASLLTFWNPPTTAQLTTESMPFNVAEGKEVLLLV |
| | HNLPQQLFGYSWYKGERVDGNRQIVGYAIGTQQATPGPANSGRETIYPNASLLIQN |
| | VTQNDTGFYTLQVIKSDLVNEEATGQFHVYPELPKPSISSNNSNPVEDKDAVAFTCE |
| | PETQDTTYLWWINNQSLPVSPRLQLSNGNRTLTLLSVTRNDTGPYECEIQNPVSAN |
| | RSDPVTLNVTYGPDTPTISPSDTYYRPGANLSLSCYAASNPPAQYSWLINGTFQQST |
| | QELFIPNITVNNSGSYTCHANNSVTGCNRTTVKTIIVTELSPVVAKPQIKASKTTVTG |
| | DKDSVNLTCSTNDTGISIRWFFKNQSLPSSERMKLSQGNTTLSINPVKREDAGTYW |
| | CEVFNPISKNQSDPIMLNVNYNALPQENGLSPGAIAGIVIGVVALVALIAVALACFL |
| | HFGKTGRASDQRDLTEHKPSVSNHTQDHSNDPPNKMNEVTYSTLNFEAQQPTQPT |
| | SASPSLTATEIIYSEVKKQ (SEQ ID NO: 64) |
| Human | >NM_007048.6 Homo sapiens butyrophilin subfamily 3 member |
| BTN3A1 | A1 (BTN3A1), transcript variant 1, mRNA, nucleic acid sequence |
| | ATTCCTCACGATGACCCGACAGTCTCTGCTTTCTTTTTCCTTTCTTCCAGAAGGA |
| | GATTTAACCATAGTAGAAAGAATGGAGAACTATTAACTGCCTTTCTTCTGTGGG |
| | CTGTGATTTTCAGAGGGGAATGCTAAGAGGTGATTTTCAATGTTGGGACTCAAA |
| | GGTGAAGACACTGAAGGACAGAATTTTTGGCAGAGGAAAGATCTTCTTCGGTC |
| | ACCATACTTGAGTTAGCTCTAGGGAAGTGGAGGTTTCCATTTGGAATTCTATAG |
| | CTTCTTCCAGGTCATAGTGTCTGCCCCCCACCTTCCAGTATCTCCTGATATGCAG |
| | CATGAATGAAAATGGCAAGTTTCCTGGCCTTCCTTCTGCTCAACTTTCGTGTCTG |
| | CCTCCTTTTGCTTCAGCTGCTCATGCCTCACTCAGCTCAGTTTTCTGTGCTTGGA |
| | CCCTCTGGGCCCATCCTGGCCATGGTGGGTGAAGACGCTGATCTGCCCTGTCAC |
| | CTGTTCCCGACCATGAGTGCAGAGACCATGGAGCTGAAGTGGGTGAGTTCCAG |
| | CCTAAGGCAGGTGGTGAACGTGTATGCAGATGGAAAGGAAGTGGAAGACAGGC |
| | AGAGTGCACCGTATCGAGGGAGAACTTCGATTCTGCGGGATGGCATCACTGCA |
| | GGGAAGGCTGCTCTCCGAATACACAACGTCACAGCCTCTGACAGTGGAAAGTA |
| | CTTGTGTTATTTCCAAGATGGTGACTTCTATGAAAAAGCCCTGGTGGAGCTGAA |
| | GGTTGCAGCACTGGGTTCTGATCTTCACGTTGATGTGAAGGGTTACAAGGATGG |
| | AGGGATCCATCTGGAGTGCAGGTCCACTGGCTGGTACCCCCAACCCCAAATACA |
| | GTGGAGCAACAACAAGGGAGAGAACATCCCGACTGTGGAAGCACCTGTGGTTG |
| | CAGACGGAGTGGGCCTGTATGCAGTAGCAGCATCTGTGATCATGAGAGGCAGC |
| | TCTGGGGAGGGTGTATCCTGTACCATCAGAAGTTCCCTCCTCGGCCTGGAAAAG |
| | ACAGCCAGCATTTCCATCGCAGACCCCTTCTTCAGGAGCGCCCAGAGGTGGATC |
| | GCCGCCCTGGCAGGGACCCTGCCTGTCTTGCTGCTGCTTCTTGGGGGAGCCGGT |
| | TACTTCCTGTGGCAACAGCAGGAGGAAAAAAAGACTCAGTTCAGAAAGAAAAA |
| | GAGAGAGCAAGAGTTGAGAGAAATGGCATGGAGCACAATGAAGCAAGAACAA |
| | AGCACAAGAGTGAAGCTCCTGGAGGAACTCAGATGGAGAAGTATCCAGTATGC |
| | ATCTCGGGGAGAGAGACATTCAGCCTATAATGAATGGAAAAAGGCCCTCTTCA |
| | AGCCTGCGGATGTGATTCTGGATCCAAAAACAGCAAACCCCATCCTCCTTGTTT |
| | CTGAGGACCAGAGGAGTGTGCAGCGTGCCAAGGAGCCCCAGGATCTGCCAGAC |
| | AACCCTGAGAGATTTAATTGGCATTATTGTGTTCTCGGCTGTGAGAGCTTCATAT |
| | CAGGGAGACATTACTGGGAGGTGGAGGTAGGGGACAGGAAAGAGTGGCATAT |
| | AGGGGTGTGCAGTAAGAATGTGCAGAGAAAAGGCTGGGTCAAAATGACACCTG |
| | AGAATGGATTCTGGACTATGGGGCTGACTGATGGGAATAAGTATCGGACTCTA |
| | ACTGAGCCCAGAACCAACCTGAAACTTCCTAAGCCCCCTAAGAAAGTGGGGGT |
| | CTTCCTGGACTATGAGACTGGAGATATCTCATTCTACAATGCTGTGGATGGATC |
| | GCATATTCATACTTTCCTGGACGTCTCCTTCTCTGAGGCTCTATATCCTGTTTTC |
| | AGAATTTTGACCTTGGAGCCCACGGCCCTGACTATTTGTCCAGCGTGAAAAGAA |
| | GAAGAGAGTTCCTCCAATTCTGACCGAGTGCTGATCATTCCCTAGAGACACCAG |
| | TAACCCCGGGCTTAGCTAACGAAAGTGGGGAGCCTCAGGCTGAAGTAACTTTTC |
| | TCTGCTTCTCCCTGCCCAGCTCAGAGCTGAGGGCCTCCCCCTCCACAGCAACCA |
| | ATCACAACCATAAAGCTACAAGCACGCACTGAAGCACTTTACTGATACTCATTC |
| | AATTATTCATATGACAGTTGTTTGAGTTTGGTACCATCTTATTTTCCCCTTATAC |
| | AGATAAGGAAACTGGGGTGCAGAAAAGTGAATTGACTACAAAGTAGACATGAC |
| | TAGTTAACAACACAGCTGGGATCTAAACAGCAATAACTAACATTAATGGAGAA |
| | CTTAAAATGCTCTGAGTGCTGTGTTATGAGCTTTGGTGGATGTCACTCCTTTAAT |
| | CCTCGCAACACCCTGTCGGGTAGTCTCATTTAGCAAGTATGGAAGTTGAGGCAG |
| | GGCAACATTAAGCAACTTACATAACTCATGCAGTAATTTCTGCAGTTGGGAGAT |
| | GTTCAGCTTCAGTCCCCGGCCCTATGGCCGTTCTTTTCCACCCTGTTTCTTCCCC |
| | CATAGGAAGAACCCACCTGTAGCCCTGAGGTTCTTTTCCCAGGATGGCTCCAGG |
| | ATAAGGATCACTGTAGGTGGTTGTGGAGTTGACACCCCTGTTGACTCCTTCCCA |
| | GCTGATTGTCAGAGCCTTAGACCCAGCACGCCTTGGATTAGCTCTGCAGAGTGT |
| | CTTGGTTGAGAGAATAACCTCACCGTACCCACATGACACGTGATTTGGAAAGAG |
| | ACTAGAGGCCACACTTGATAAATCATGGGGAACAGATGTGTTCCACCCAACAA |
| | ATGTGATAAGTGATCATGCAGCCAGAGCCAGCCTTCCTTCAATCAAGGTTTCCA |
| | GGCAGAGCAAATACCCTAGAGATTCTCTGTGATATAGGAAATTTGGATGAAGG |
| | GAGCTAGAAGAAATACAGGGATTTTTTTTTTTTTTTAAGATGGAGTCTTACTCTG |
| | TTGCTAGGCTGGAGTGCAGTGGTGCGATCTCAGCTCCCTGCAACCTCCACCTCC |
| | TGGGTTCAAACAATTCTCCTGCCTCAGCCTCCCGAGTACTGGGAATATAGGTGC |
| | ACGCCACCACACCCAACAAATTTTTGTACTTTTAGTACAGATGAGGGTTCACTA |
| | TGTTGGCCAGGATGGTCTCGATCTCTTGACCTCATGATCCACCCACCTCGGTCTC |
| | CCAAAGTGCTGGGATTACAGGCTTGAGCCACCGGGTGACCGGCTTACAGGGAT |
| | ATTTTTAATCCCGTTATGGACTCTGTCTCCAGGAGAGGGGTCTATCCACCCCTGC |
| | TCATTGGTGGATGTTAAACCAATATTCCTTTCAACTGCTGCCTGCTAGGGAAAA |
| | ACTACTCCTCATTATCATCATTATTATTGCTCTCCACTGTATCCCCTCTACCTGG |
| | CATGTGCTTGTCAAGTTCTAGTTGTTCAATAAATTTGTTAATAATGCTGA |
| | (SEQ ID NO: 65) |
| | >NP_008979.3 butyrophilin subfamily 3 member A1 isoform |
| | a precursor [Homo sapiens], amino acid sequence |
| | MKMASFLAFLLLNFRVCLLLLQLLMPHSAQFSVLGPSGPILAMVGEDADLPCHLFP |
| | TMSAETMELKWVSSSLRQVVNVYADGKEVEDRQSAPYRGRTSILRDGITAGKAAL |
| | RIHNVTASDSGKYLCYFQDGDFYEKALVELKVAALGSDLHVDVKGYKDGGIHLEC |
| | RSTGWYPQPQIQWSNNKGENIPTVEAPVVADGVGLYAVAASVIMRGSSGEGVSCTI |
| | RSSLLGLEKTASISIADPFFRSAQRWIAALAGTLPVLLLLLGGAGYFLWQQQEEKKT |
| | QFRKKKREQELREMAWSTMKQEQSTRVKLLEELRWRSIQYASRGERHSAYNEWK |
| | KALFKPADVILDPKTANPILLVSEDQRSVQRAKEPQDLPDNPERFNWHYCVLGCES |
| | FISGRHYWEVEVGDRKEWHIGVCSKNVQRKGWVKMTPENGFWTMGLTDGNKYR |
| | TLTEPRTNLKLPKPPKKVGVFLDYETGDISFYNAVDGSHIHTFLDVSFSEALYPVFRI |
| | LTLEPTALTICPA (SEQ ID NO: 66) |
| Human | >NM_007047.5 Homo sapiens butyrophilin subfamily 3 member |
| BTN3A2 | A2 (BTN3A2), transcript variant 1, mRNA, nucleic acid sequence |
| | GACTCTTACTGTTTCTCATGGTGAGAAGACAATATTTGCTTTCTCTTTTTCCTTTC |
| | TTCCGGATGAGAGGCTAAGCCATAATAGAAAGAATGGAGAATTATTGATTGAC |
| | CGTCTTTATTCTGTGGGCTCTGATTCTCCAATGGGAATACCAAGGGATGGTTTTC |
| | CATACTGGAACCCAAAGGTAAAGACACTCAAGGACAGACATTTTTGGCAGAGC |
| | ATAGATGAAAATGGCAAGTTCCCTGGCTTTCCTTCTGCTCAACTTTCATGTCTCC |
| | CTCCTCTTGGTCCAGCTGCTCACTCCTTGCTCAGCTCAGTTTTCTGTGCTTGGAC |
| | CCTCTGGGCCCATCCTGGCCATGGTGGGTGAAGACGCTGATCTGCCCTGTCACC |
| | TGTTCCCGACCATGAGTGCAGAGACCATGGAGCTGAAGTGGGTAAGTTCCAGC |
| | CTAAGGCAGGTGGTGAACGTGTATGCAGATGGAAAGGAAGTGGAAGACAGGC |
| | AGAGTGCACCGTATCGAGGGAGAACTTCGATTCTGCGGGATGGCATCACTGCA |
| | GGGAAGGCTGCTCTCCGAATACACAACGTCACAGCCTCTGACAGTGGAAAGTA |
| | CTTGTGTTATTTCCAAGATGGTGACTTCTATGAAAAAGCCCTGGTGGAGCTGAA |
| | GGTTGCAGCACTGGGTTCTAATCTTCACGTCGAAGTGAAGGGTTATGAGGATGG |
| | AGGGATCCATCTGGAGTGCAGGTCCACCGGCTGGTACCCCCAACCCCAAATAC |
| | AGTGGAGCAACGCCAAGGGAGAGAACATCCCAGCTGTGGAAGCACCTGTGGTT |
| | GCAGATGGAGTGGGCCTATATGAAGTAGCAGCATCTGTGATCATGAGAGGCGG |
| | CTCCGGGGAGGGTGTATCCTGCATCATCAGAAATTCCCTCCTCGGCCTGGAAAA |
| | GACAGCCAGCATTTCCATCGCAGACCCCTTCTTCAGGAGCGCCCAGCCCTGGAT |
| | CGCAGCCCTGGCAGGGACCCTGCCTATCTTGCTGCTGCTTCTCGCCGGAGCCAG |
| | TTACTTCTTGTGGAGACAACAGAAGGAAATAACTGCTCTGTCCAGTGAGATAGA |
| | AAGTGAGCAAGAGATGAAAGAAATGGGATATGCTGCAACAGAGCGGGAAATA |
| | AGCCTAAGAGAGAGCCTCCAGGAGGAACTCAAGAGGAAAAAAATCCAGTACTT |
| | GACTCGTGGAGAGGAGTCTTCGTCCGATACCAATAAGTCAGCCTGATGCTCTAA |
| | TGGAAAAATGGCCCTCTTCAAGCCTGGTGAGGAAATGCTTCAGATGAGGCTCCA |
| | CCTTGTTAAATAAATTGGATGTATGGAAAAATAGACTGCAGAAAAGGGGAACT |
| | CATTTAGCTCACGAGTGGTCGAGTGAAGATTGAAAATTAACCTCTGAGGGCCAG |
| | CACAGCAGCTCATGCCTGTAATCCTAGCACTTTGGAAGGCTGAGGAGGGCGGA |
| | TCACAAGGTCAGGAGATCAAGACCATCCTGGCTAACACGGTGAAACCCCGTCT |
| | CTACTAAAAATACAAAAAATAAAAAATTAGCCGGGCATGGTGACGGGCACCTG |
| | TAGTCCCAGCTACTCGGGAGGCTGAGGCAGGAGAATGGCATGAACCCGGAAGG |
| | CAGAGCTTGCAGTGAGCCGAGATCACGCCACTGCACTCCAGCCTGGGAGACAG |
| | AGCGAGACTCTGTCTCAAGAAAAAAAAAAAAAAAAAAAAAGAAAAGAAAATT |
| | AACCTCTGAGTATAAAGCATCAGTGGGCAGAATCAATGTGGGGAGGGAAACAA |
| | CAAAAATGTAGAAAGAGGATCCTTGTTGCTTCTTGGGGCCGCATCAGGGTATTG |
| | GGTTAGGCAGATACTGACCTTACTTTCATTTCCCCTCTGGTCACTAGACCCCTGG |
| | GGCTTTCACCAATGACATTGATGAGAGAATCACATTCAGGGCAGGCTAGGGAC |
| | ACGGGGTTCTGGAAGGACCTCCTCAGCATGGCCCAAGCCTTGCATGCTGTGGCT |
| | CTTAAATCCAGGAAAAATGGCTGACCCCATGGACACCTCCTCAAACTCTCTGCA |
| | GCAGATGTAATTCTGTATCCAGACATGGCAAATGCCATCCTCCTTGTTTCTGAG |
| | GACCAGAGGAGTGTACAGCGTGCTGAGGAGCCCCATGACCTACCAGACAACCC |
| | TGAGAGATTTGAATGGCGTTACTGTGTGCTTGGCTGTGAAAGCTTCATGTCAGA |
| | GAGACACTACTGGGAGGTGGAAGTGGGGGACAGAAAAGAGTGGCATATTGGG |
| | GTATGTAGTAAGAACGTGGAGAGGAAAAAAGTTTGGGTCAAAATGACACCGGA |
| | GAACGGATACTGGACTATGGGCCTGACTGATGGGAATAAGTATCGGGCTCTCA |
| | CTGAGCCCAGAACCAACCTGAAACTTCCTGAGCCTCCTAGGAAAGTGGGGGTC |
| | ATCCTGGACTATGAGACTGGACATATCTCGTTCTACAATGCCACGGATGGATCT |
| | CATATCTACACATTTCTGCACGCCTCTTCCTCTGAGCCTCTGTATCCTGTATTCA |
| | GAATTTTGACCTTGGAGCCCACTGCCCTGACCGTTTGCCCAATACCAAAAGTAG |
| | AGAGTTCCCCCGATCCCGACCTAGTGCCTGATCATTCCCTGGAGATACCACTGA |
| | CCCCAGGCTTAGCTAATGAAAGTGGGGAGCCTCAGGCTGAAGTAACATCTCTGC |
| | TTCTCCCTGCCCAGCCTGGAGCTAAGGGTCTCACCCTCCACAACAGCCAGTCAG |
| | AACCATAAAGCTACAGGCACACACTGAAGCACTTTACTGATATTCATTCAATTA |
| | TTCCATAGGACAGTTGTTTGAGTTTGGTGCCACCTTATTGGCCCCTTTATACAGA |
| | TAAGGAAACTGGGGTGTAGAAAAGTGTATTGACTTTACAAAGCAGACAGGAAT |
| | AGTGAACAACAGAGCTGGGATCTGAACAACAATGACTAACATTAATGGAGAAT |
| | TTAAAACGTTCTGAGTGCTGTGTTATGAGCTTTGGTGGGTGTCACTCCTTTAATC |
| | CTCACAACACCCTGTCAGGTAGTCTCATTTGGCAAGTATGGAAGCAGAGGCAG |
| | GGCAACATTAAGTAGCTTACATAACTCACACGGTAATTTGTGCAGTTGGGAGAT |
| | GTTCAGCTTCAGTCCCTGGCCAATTGCCCGTTCTTTTCCAGCCTGATTTTTCCTG |
| | CATGGGAAGAGCCCACATGTAGCCCTGAGGTTCCCTTCCCAGGACAGCTCCAGG |
| | ATCGAGATCACTGTGAGTGGTTGTGGAGTTAAGACCCCTATGGACTCCTTCCCA |
| | GCTGATTATCAGAGCCTTAGACCCAGCACTCCTTGGATTGGCTCTGCAGAGTGT |
| | CTTGGTTGAGAGAATAACGTTGCAGTTCCCACAGGGCATGTGACTTTGAAAGAG |
| | ACTAGAGGCCACACTCAGTTAATAATGGGGCACAGATGTGTTCCCACCCAACA |
| | AATGTGATAAGTGATCGTGCAGCCAGAGCCAGCCTTCCTTCAGTCAAGGTTTCC |
| | AGGCAGAGCAAATACCCTAGAGATTCTCTGTAATATTGGTAATTTGGATGAAGG |
| | AAGCTAGAAGAATTACAGGGATGTTTTTAATCCCACTATGGACTCAGTCTCCTG |
| | GAAAAGGATCTGTCCACTCCTGGTCATTGGTGGATGTTAAACCCATATTCCTTTC |
| | AACTGCTGCCTGCTAGGGAAAACTGCTCCTCATTATCATCACTATTATTGCTCAC |
| | CACTGTATCCCCTCTACTGGGCAAGTGCTTGTCAAGTTCTAGTTGTTCAATAAAT |
| | TTGTTAATAATGCTGA (SEQ ID NO: 67) |
| | >NP_008978.2 butyrophilin subfamily 3 member A2 isoform |
| | a precursor [Homo sapiens], amino acid sequence |
| | MKMASSLAFLLLNFHVSLLLVQLLTPCSAQFSVLGPSGPILAMVGEDADLPCHLFPT |
| | MSAETMELKWVSSSLRQVVNVYADGKEVEDRQSAPYRGRTSILRDGITAGKAALR |
| | IHNVTASDSGKYLCYFQDGDFYEKALVELKVAALGSNLHVEVKGYEDGGIHLECR |
| | STGWYPQPQIQWSNAKGENIPAVEAPVVADGVGLYEVAASVIMRGGSGEGVSCIIR |
| | NSLLGLEKTASISIADPFFRSAQPWIAALAGTLPILLLLLAGASYFLWRQQKEITALSS |
| | EIESEQEMKEMGYAATEREISLRESLQEELKRKKIQYLTRGEESSSDTNKSA |
| | (SEQ ID NO: 68) |
| Human | >NM_007049.5 Homo sapiens butyrophilin subfamily 2 member |
| BTN2A1 | A1 (BTN2A1), transcript variant 1, mRNA, nucleic acid sequence |
| | AGATTTCGTTTCCTGCATCTCCAAACATGGCGACCTAGGAGAAGGGGAAGAAC |
| | AATTTTTTCTCCTCTTTTGGGAAGGTTTGTGTCTAGTAGTGCCTGTGCCCCTGGG |
| | CAGATTGGAGAGAAGAGGGACGACTGGAGAATCGTCGAGAACCAGCGGAGAA |
| | AAGAAAAAGCAACGTTTAATTCTAGAAGGCCTCCTGTCCCTGCCTGCTCTGGGT |
| | GCTCATGGAATCAGCTGCTGCCCTGCACTTCTCCCGGCCAGCCTCCCTCCTCCTC |
| | CTCCTCCTCAGCCTGTGTGCACTGGTCTCAGCCCAGTTTATTGTCGTGGGGCCCA |
| | CTGATCCCATCTTGGCCACGGTTGGAGAAAACACTACGTTACGCTGCCATCTGT |
| | CACCCGAGAAAAATGCTGAGGACATGGAGGTGCGGTGGTTCCGGTCTCAGTTCT |
| | CCCCCGCAGTGTTTGTGTATAAAGGTGGCAGAGAGAGAACAGAGGAGCAGATG |
| | GAGGAGTACCGAGGAAGAACCACCTTTGTGAGCAAAGACATCAGCAGGGGCAG |
| | CGTGGCCCTGGTCATACACAACATCACAGCCCAGGAAAACGGCACCTACCGCT |
| | GTTACTTCCAAGAAGGCAGGTCCTACGATGAGGCCATCCTGCACCTCGTAGTGG |
| | CAGGACTAGGCTCTAAGCCCCTCATTTCAATGAGGGGCCATGAAGACGGGGGC |
| | ATCCGGCTGGAGTGCATATCTAGAGGGTGGTACCCAAAGCCCCTCACAGTGTGG |
| | AGGGACCCCTACGGTGGGGTTGCGCCTGCCCTGAAAGAGGTCTCCATGCCTGAT |
| | GCAGACGGCCTCTTCATGGTCACCACGGCTGTGATCATCAGAGACAAGTCTGTG |
| | AGGAACATGTCCTGCTCTATCAACAACACCCTGCTCGGCCAGAAGAAAGAAAG |
| | TGTCATTTTTATTCCAGAATCCTTTATGCCCAGTGTGTCTCCCTGTGCAGTGGCC |
| | CTGCCTATCATTGTGGTTATTCTGATGATACCCATTGCCGTATGCATCTATTGGA |
| | TCAACAAACTCCAAAAGGAAAAAAAGATTCTGTCAGGGGAAAAGGAGTTTGAA |
| | CGGGAAACAAGAGAAATTGCTCTAAAGGAACTGGAGAAAGAACGTGTGCAAA |
| | AAGAGGAAGAACTTCAAGTAAAAGAGAAACTTCAAGAAGAATTGCGATGGAG |
| | AAGAACATTCTTACATGCTGTTGATGTGGTCCTGGATCCAGACACCGCTCATCC |
| | CGATCTCTTCCTGTCAGAGGACCGGAGAAGTGTGAGAAGGTGCCCCTTCAGGCA |
| | CCTAGGGGAGAGCGTGCCTGACAACCCAGAGAGATTCGACAGTCAGCCTTGTG |
| | TCCTAGGCCGGGAGAGCTTCGCTTCAGGGAAACATTACTGGGAGGTGGAGGTG |
| | GAAAACGTGATTGAGTGGACTGTGGGGGTCTGTAGAGACAGTGTTGAGAGGAA |
| | AGGGGAGGTCCTGCTGATTCCTCAGAATGGCTTCTGGACCTTGGAGATGCATAA |
| | AGGGCAATACCGGGCCGTGTCCTCCCCTGATAGGATTCTCCCTTTGAAGGAGTC |
| | CCTTTGCCGGGTGGGCGTCTTCCTGGACTATGAAGCTGGAGATGTCTCCTTCTAC |
| | AACATGAGGGACAGATCGCACATCTACACATGTCCCCGTTCAGCCTTTTCCGTG |
| | CCTGTGAGGCCCTTCTTCAGGTTGGGGTGTGAGGACAGCCCCATCTTCATCTGC |
| | CCTGCACTCACAGGAGCCAATGGGGTCACGGTGCCTGAAGAGGGCCTGACACT |
| | TCACAGAGTGGGGACCCACCAGAGCCTATAGAATCAATTCCTTGGTCTCACAGC |
| | CATGTAGACAAGCCCTGGTCATCTCAGCAGCCACCGCACAACACCCCTGGTGGA |
| | AGACACGCCCTCCTCCCCTCTGGTCACACAAGAGAACATCTTCCAGCTGCCTCT |
| | TTCACACCCACTACAGACCTCAGCCCCAGTTTTCTCCTCCTCACTAGGCTGTGTT |
| | TTTAGTAGTTCCTTTGCTTGTAACTATGGGATGGGATCCAGGCATAGGGAACTA |
| | GTTGTTACACAGCTCCCAGCCAAGAAGAAAGTGTGAGAAGTTGATGGGCAGCA |
| | AACCTGCTGTTTAACATCAGGGTGACCACATTAAGCCCAGTATTCCAGTTGGCA |
| | CCAGAAGATATGGACTTGGAATGAGGCCTACAGGGTTCACCAGGATGTAAGAG |
| | GAGAGAGGAATCCACAGGACCACCAGAGAGGAGAGGGAACCAGATATGCAGA |
| | TCAGAGATAGAGGAAGTGGAACCAGAGAGCTGGGAGGGACCAAGGTTGTAAG |
| | GGTGGCTAAGTCCCACCATAACAGCTAAGGGGACCTGGGAGATGATGGCTCAT |
| | TTCCACCCAGCCCCAGGATTTCCAGAGCGCACATCCACAGGCCTGGACCTGGGA |
| | TGAAGATGAATGAAGAACATGGATGCACGTGGATGTAGTTTGGCTCAGGTGTC |
| | CCTGCAGTTGGCAAGGAGTCAGTACTCAGTCCCTGAGTGTGGCTGAAATTTGAG |
| | GTCCTGGCTGAGCCAAGGAGTAATGGACCAGATCTACCTCAGTATTCAAGTTCA |
| | GTGGGGACACCAGTGGCTTCAAACTTCCTGGTTTCATGATATCTTGAGACGCCT |
| | TACAAATGATGGAGGATTCCAAAGAGTTTTTGTTTATTTGGGTTAATATTTGTTG |
| | GTATTTATGGCATTTGAGATTGAAACTAAGAAATGTTTTAATTTATTACCTTTAC |
| | AACATTTATTTACATTACATACATACATTTACAACATTTATTAATTTATATTAAA |
| | ATAGCATGAATAAGCCAATTATAGGTTAATATAAGTAGAATGTTTGTGAAAAAT |
| | AAGTATGGTATCCAAAGCAAAATAAATTTTATTGTGAAGTGTG (SEQ ID NO: 69) |
| | >NP_008980.1 butyrophilin subfamily 2 member Al isoform 1 |
| | precursor [Homo sapiens], amino acid sequence |
| | MESAAALHFSRPASLLLLLLSLCALVSAQFIVVGPTDPILATVGENTTLRCHLSPEKN |
| | AEDMEVRWFRSQFSPAVFVYKGGRERTEEQMEEYRGRTTFVSKDISRGSVALVIHN |
| | ITAQENGTYRCYFQEGRSYDEAILHLVVAGLGSKPLISMRGHEDGGIRLECISRGWY |
| | PKPLTVWRDPYGGVAPALKEVSMPDADGLFMVTTAVIIRDKSVRNMSCSINNTLL |
| | GQKKESVIFIPESFMPSVSPCAVALPIIVVILMIPIAVCIYWINKLQKEKKILSGEKEFE |
| | RETREIALKELEKERVQKEEELQVKEKLQEELRWRRTFLHAVDVVLDPDTAHPDLF |
| | LSEDRRSVRRCPFRHLGESVPDNPERFDSQPCVLGRESFASGKHYWEVEVENVIEW |
| | TVGVCRDSVERKGEVLLIPQNGFWTLEMHKGQYRAVSSPDRILPLKESLCRVGVFL |
| | DYEAGDVSFYNMRDRSHIYTCPRSAFSVPVRPFFRLGCEDSPIFICPALTGANGVTV |
| | PEEGLTLHRVGTHQSL (SEQ ID NO: 70) |
| Human | >NM_001040462.3 Homo sapiens butyrophilin like 8 (BTNL8), |
| BTNL8 | transcript variant 2, mRNA, nucleic acid sequence |
| | AGAACAGCGCAGTTTGCCCTCCGCTCACGCAGAGCCTCTCCGTGGCTTCCGCAC |
| | CTTGAGCATTAGGCCAGTTCTCCTCTTCTCTCTAATCCATCCGTCACCTCTCCTG |
| | TCATCCGTTTCCATGCCGTGAGGTCCATTCACAGAACACATCCATGGCTCTCAT |
| | GCTCAGTTTGGTTCTGAGTCTCCTCAAGCTGGGATCAGGGCAGTGGCAGGTGTT |
| | TGGGCCAGACAAGCCTGTCCAGGCCTTGGTGGGGGAGGACGCAGCATTCTCCT |
| | GTTTCCTGTCTCCTAAGACCAATGCAGAGGCCATGGAAGTGCGGTTCTTCAGGG |
| | GCCAGTTCTCTAGCGTGGTCCACCTCTACAGGGACGGGAAGGACCAGCCATTTA |
| | TGCAGATGCCACAGTATCAAGGCAGGACAAAACTGGTGAAGGATTCTATTGCG |
| | GAGGGGCGCATCTCTCTGAGGCTGGAAAACATTACTGTGTTGGATGCTGGCCTC |
| | TATGGGTGCAGGATTAGTTCCCAGTCTTACTACCAGAAGGCCATCTGGGAGCTA |
| | CAGGTGTCAGCACTGGGCTCAGTTCCTCTCATTTCCATCACGGGATATGTTGAT |
| | AGAGACATCCAGCTACTCTGTCAGTCCTCGGGCTGGTTCCCCCGGCCCACAGCG |
| | AAGTGGAAAGGTCCACAAGGACAGGATTTGTCCACAGACTCCAGGACAAACAG |
| | AGACATGCATGGCCTGTTTGATGTGGAGATCTCTCTGACCGTCCAAGAGAACGC |
| | CGGGAGCATATCCTGTTCCATGCGGCATGCTCATCTGAGCCGAGAGGTGGAATC |
| | CAGGGTACAGATAGGAGATACCTTTTTCGAGCCTATATCGTGGCACCTGGCTAC |
| | CAAAGTACTGGGAATACTCTGCTGTGGCCTATTTTTTGGCATTGTTGGACTGAA |
| | GATTTTCTTCTCCAAATTCCAGTGGAAAATCCAGGCGGAACTGGACTGGAGAAG |
| | AAAGCACGGACAGGCAGAATTGAGAGACGCCCGGAAACACGCAGTGGAGGTG |
| | ACTCTGGATCCAGAGACGGCTCACCCGAAGCTCTGCGTTTCTGATCTGAAAACT |
| | GTAACCCATAGAAAAGCTCCCCAGGAGGTGCCTCACTCTGAGAAGAGATTTAC |
| | AAGGAAGAGTGTGGTGGCTTCTCAGAGTTTCCAAGCAGGGAAACATTACTGGG |
| | AGGTGGACGGAGGACACAATAAAAGGTGGCGCGTGGGAGTGTGCCGGGATGAT |
| | GTGGACAGGAGGAAGGAGTACGTGACTTTGTCTCCCGATCATGGGTACTGGGTC |
| | CTCAGACTGAATGGAGAACATTTGTATTTCACATTAAATCCCCGTTTTATCAGC |
| | GTCTTCCCCAGGACCCCACCTACAAAAATAGGGGTCTTCCTGGACTATGAGTGT |
| | GGGACCATCTCCTTCTTCAACATAAATGACCAGTCCCTTATTTATACCCTGACAT |
| | GTCGGTTTGAAGGCTTATTGAGGCCCTACATTGAGTATCCGTCCTATAATGAGC |
| | AAAATGGAACTCCCATAGTCATCTGCCCAGTCACCCAGGAATCAGAGAAAGAG |
| | GCCTCTTGGCAAAGGGCCTCTGCAATCCCAGAGACAAGCAACAGTGAGTCCTCC |
| | TCACAGGCAACCACGCCCTTCCTCCCCAGGGGTGAAATGTAGGATGAATCACAT |
| | CCCACATTCTTCTTTAGGGATATTAAGGTCTCTCTCCCAGATCCAAAGTCCCGCA |
| | GCAGCCGGCCAAGGTGGCTTCCAGATGAAGGGGGACTGGCCTGTCCACATGGG |
| | AGTCAGGTGTCATGGCTGCCCTGAGCTGGGAGGGAAGAAGGCTGACATTACAT |
| | TTAGTTTGCTCTCACTCCATCTGGCTAAGTGATCTTGAAATACCACCTCTCAGGT |
| | GAAGAACCGTCAGGAATTCCCATCTCACAGGCTGTGGTGTAGATTAAGTAGAC |
| | AAGGAATGTGAATAATGCTTAGATCTTATTGATGACAGAGTGTATCCTAATGGT |
| | TTGTTCATTATATTACACTTTCAGTAA (SEQ ID NO: 71) |
| | >NP_001035552.1 butyrophilin-like protein 8 isoform 2 |
| | precursor [Homo sapiens], amino acid sequence |
| | MALMLSLVLSLLKLGSGQWQVFGPDKPVQALVGEDAAFSCFLSPKTNAEAMEVRF |
| | FRGQFSSVVHLYRDGKDQPFMQMPQYQGRTKLVKDSIAEGRISLRLENITVLDAGL |
| | YGCRISSQSYYQKAIWELQVSALGSVPLISITGYVDRDIQLLCQSSGWFPRPTAKWK |
| | GPQGQDLSTDSRTNRDMHGLFDVEISLTVQENAGSISCSMRHAHLSREVESRVQIG |
| | DTFFEPISWHLATKVLGILCCGLFFGIVGLKIFFSKFQWKIQAELDWRRKHGQAELR |
| | DARKHAVEVTLDPETAHPKLCVSDLKTVTHRKAPQEVPHSEKRFTRKSVVASQSF |
| | QAGKHYWEVDGGHNKRWRVGVCRDDVDRRKEYVTLSPDHGYWVLRLNGEHLY |
| | FTLNPRFISVFPRTPPTKIGVFLDYECGTISFFNINDQSLIYTLTCRFEGLLRPYIEYPSY |
| | NEQNGTPIVICPVTQESEKEASWQRASAIPETSNSESSSQATTPFLPRGEM |
| | (SEQ ID NO: 72) |
| Human | >NM_006995.5 Homo sapiens butyrophilin subfamily 2 member |
| BTN2A2 | A2 (BTN2A2), transcript variant 1, mRNA, nucleic acid sequence |
| | GGGACTTTTTGGACACCCAGAGAACAGGTCCCAGATACCGAGTCCGCAACTCC |
| | AAACATCGCGATTAATAGGAGGCCTCTGGTCTCTGCCTGCCCTGGGTGCTCATG |
| | GAACCAGCTGCTGCTCTGCACTTCTCCCTGCCAGCCTCCCTCCTCCTCCTCCTGC |
| | TCCTCCTCCTTCTCAGCCTGTGTGCACTGGTCTCAGCCCAGTTTACTGTCGTGGG |
| | GCCAGCTAATCCCATCCTGGCCATGGTGGGAGAAAACACTACATTACGCTGCCA |
| | TCTGTCACCCGAGAAAAATGCTGAGGACATGGAGGTGCGGTGGTTCCGGTCTCA |
| | GTTCTCCCCCGCAGTGTTTGTGTATAAGGGTGGGAGAGAGAGAACAGAGGAGC |
| | AGATGGAGGAGTACCGGGGAAGAATCACCTTTGTGAGCAAAGACATCAACAGG |
| | GGCAGCGTGGCCCTGGTCATACATAACGTCACAGCCCAGGAGAATGGGATCTA |
| | CCGCTGTTACTTCCAAGAAGGCAGGTCCTACGATGAGGCCATCCTACGCCTCGT |
| | GGTGGCAGGCCTTGGGTCTAAGCCCCTCATTGAAATCAAGGCCCAAGAGGATG |
| | GGAGCATCTGGCTGGAGTGCATATCTGGAGGGTGGTACCCAGAGCCCCTCACA |
| | GTGTGGAGGGACCCCTACGGTGAGGTTGTGCCCGCCCTGAAGGAGGTTTCCATC |
| | GCTGATGCTGACGGCCTCTTCATGGTCACCACAGCTGTGATCATCAGAGACAAG |
| | TATGTGAGGAATGTGTCCTGCTCTGTCAACAACACCCTGCTCGGCCAGGAGAAG |
| | GAAACTGTCATTTTTATTCCAGAATCCTTTATGCCCAGCGCATCTCCCTGGATGG |
| | TGGCCCTAGCTGTCATCCTGACCGCATCTCCCTGGATGGTGTCCATGACTGTCAT |
| | CCTGGCTGTTTTCATCATCTTCATGGCTGTCAGCATCTGTTGCATCAAGAAACTT |
| | CAAAGGGAAAAAAAGATTCTGTCAGGGGAAAAGAAAGTTGAACAAGAGGAAA |
| | AAGAAATTGCACAGCAACTTCAAGAAGAATTGCGATGGAGAAGAACATTCTTA |
| | CATGCTGCTGATGTGGTCCTGGATCCAGACACCGCTCATCCCGAGCTCTTCCTG |
| | TCAGAGGACCGGAGAAGTGTGAGGCGGGGCCCCTACAGGCAGAGAGTGCCTGA |
| | CAACCCAGAGAGATTCGACAGTCAGCCTTGTGTCCTGGGATGGGAGAGCTTCGC |
| | CTCAGGGAAACATTACTGGGAGGTGGAGGTGGAAAACGTGATGGTGTGGACTG |
| | TGGGGGTCTGCAGACACAGTGTTGAGAGGAAAGGGGAGGTCCTGCTGATTCCT |
| | CAGAATGGCTTCTGGACCCTGGAGATGTTTGGAAACCAATACCGGGCCCTGTCC |
| | TCCCCTGAGAGGATTCTCCCTTTGAAGGAGTCCCTTTGCCGGGTGGGCGTCTTC |
| | CTGGACTATGAAGCTGGAGATGTCTCCTTCTACAACATGAGGGACAGATCGCAC |
| | ATCTACACATGTCCCCGTTCAGCCTTTACTGTGCCTGTGAGGCCCTTCTTCAGGT |
| | TAGGGTCTGATGACAGCCCCATCTTCATCTGCCCTGCACTCACAGGAGCCAGTG |
| | GGGTCATGGTGCCTGAAGAGGGCCTGAAACTTCACAGAGTGGGGACCCACCAG |
| | AGCCTATAGAATCAATTCCTTGGACTCACAGCCATGCAGATAAGCCCTGGCCAT |
| | CTCAGCAGCCACCGCACAACCCCCCTAATGAAAGACACGCCCTCCTCCCCTCTG |
| | GTCACGTAAGAGAACATCTTCCAGCTGCCTTTTTCACACCCACTCCAGCCCTCT |
| | GCCCCAGTTTTCTCCTCCTCACTAGTCTGTGGCTTTAGTAGTTCCTTTGCTTGTA |
| | ATTATGGGATGGGATCCAGGCATAGGGAACTAGTTGTTTCATAGCTCCCAGTCA |
| | AAAAGAAAGTGAGAGAAGCTGTTGGGCAGCGAACCTACTGTTTAAAATCAGGA |
| | TAACCACATTAAGCCCAATATGCCAGTTGGCACCAGATGCTGTGGACTTGGAAT |
| | GAGGCCAACAGGGTTCACCAGGATGAGAGAGGAGAGAGGAATCCACAGGACC |
| | ACCAGAAGGGAGAGGGAACCAGATATGCAGATCAGAGATAGAGGAAGTGGAA |
| | CCAGAGAGCTGGGAGGGACCAAGGTTGTAAGGATGGCTAAGTCCCACCATAAG |
| | AGCTAAAGGGTCCTGGGAGATGATGGCTCATTTCCACCCAACCCCAGGATTTCC |
| | ACAGCACACACCCACAGGCCTGGACCTGGGATGAAGATGAATGAAGAACATGG |
| | ACTCATGTGGATGTGGTTTGGCTCAGATGTCCCTGCAATAAACAAGGGGTCAGT |
| | ACTTAGTCCCTGAGTGTGGTTGAGGTTTGAGGTCCTGGTCGAGCAGGGCAGTAC |
| | TGGACCAGGTCTACGTCAGCATTCAGGTTCAATGGGGACACCAGTGGCTTCAAA |
| | CTTCCTGATCTAATTATGTTTTTAGACACTTAGAAGTTATTGAGGACTTTAAAGA |
| | GCTTTTGTTTATTTGGGTTAATATTTATGACATTTGACATTGAAACAAAAATTTA |
| | AAATGTTATCTTTTAATTTATGTTAAAATAGCATTAATAAATCAGTTATAGGTTA |
| | ATGTAGATAGGATGTTTTGTGAAAAAGCAATCTATTGTGTCCAAATAAAAAAAC |
| | AAAAAGTGTGACACTGGTTAACTTTTTCCAGATCTCATGTCTGGCTTAATAAGA |
| | GATATTTGTATTATCATATCTGCCTTTGTATTAAACCTATTGGTATATCATAGGT |
| | CATGTTAGCTCAAAAAAACTTTACTGCACACTACTGAGAGAATGAGATGAAAA |
| | ACGATTAATGTTTCATTATTATTATTGTGAAAATATTATTAACACTGGGGACTCC |
| | TTAAGAGTACATCAGAGTTCTCTCTAGGAATCCCAAAACCACATTTTGAAACTA |
| | GAATAGTGGATCCTGGAAGTTAATCCATGTGCTGGTTAATTTTAGATGTCAACC |
| | TGACTGGATTAAGGAATACCTAGACAGCTGGTACAACATTATTTCTGGGTGTGT |
| | CTGTGAGTGTGTTTCCAGAAGAGATTGGCAAGTGAGTCAGTGGGAAATTCTCTC |
| | CTTCTGTTGGCTGGGTGCCCAATACAACAAAAAGGCAGAGGAAAGGCAAATTC |
| | TTCTCTCCTCTGGAGCTGAGACACTCTTCTTCTTCTGCCCTTGGACATCAGAACT |
| | CCTGGCTCTCCGGCCTTTGAACTTCAGGACTTGTACCAGGAGGCCCTGGGTTCT |
| | CAGGCCTTTGGCTTTGGACTGAGAGTTACACAATCAGCTTCCCTGGTTCTGAGG |
| | CTTTCAGACTTAAACTGAGCCATGCTACCAGCATCCCAGGGTCTCCAGCCTACA |
| | GATGAGCTGTTGTGCGATTTCTTAGCCTCCATAATCACATGAGCCAATCTCCTTA |
| | ATAAATGCCTGCTCATAGATCTGTATCTACATCTATATCTGTATGTGCATCTATA |
| | TCTATGCCTATATCTATATCTATATCATATTGATTTTGTCTCTCTGGAGAACCCT |
| | GACTAATAAAATGAGGCATCTAAAA (SEQ ID NO: 73) |
| | >NP_008926.2 butyrophilin subfamily 2 member A2 isoform a |
| | precursor [Homo sapiens], amino acid sequence |
| | MEPAAALHFSLPASLLLLLLLLLLSLCALVSAQFTVVGPANPILAMVGENTTLRCHL |
| | SPEKNAEDMEVRWFRSQFSPAVFVYKGGRERTEEQMEEYRGRITFVSKDINRGSVA |
| | LVIHNVTAQENGIYRCYFQEGRSYDEAILRLVVAGLGSKPLIEIKAQEDGSIWLECIS |
| | GGWYPEPLTVWRDPYGEVVPALKEVSIADADGLFMVTTAVIIRDKYVRNVSCSVN |
| | NTLLGQEKETVIFIPESFMPSASPWMVALAVILTASPWMVSMTVILAVFIIFMAVSIC |
| | CIKKLQREKKILSGEKKVEQEEKEIAQQLQEELRWRRTFLHAADVVLDPDTAHPEL |
| | FLSEDRRSVRRGPYRQRVPDNPERFDSQPCVLGWESFASGKHYWEVEVENVMVW |
| | TVGVCRHSVERKGEVLLIPQNGFWTLEMFGNQYRALSSPERILPLKESLCRVGVFL |
| | DYEAGDVSFYNMRDRSHIYTCPRSAFTVPVRPFFRLGSDDSPIFICPALTGASGVMV |
| | PEEGLKLHRVGTHQSL (SEQ ID NO: 74) |
| Human | >NM_001732.3 Homo sapiens butyrophilin subfamily 1 member |
| BTN1A1 | A1 (BTN1A1), mRNA, nucleic acid sequence |
| | AGCTTTCTCACTTGGTAGCAGTGGCCTCTTGTGCCTTTTTCTCCAAGATCACCCA |
| | GGCTGAAGCTCCTGAGGGGACTCACATCAGTTATCTTGCTGCTCCAGAAGGGTG |
| | GGAGATGGCAGTTTTCCCAAGCTCCGGTCTCCCCAGATGTCTGCTCACCCTCAT |
| | TCTCCTCCAGCTGCCCAAACTGGATTCAGCTCCCTTTGACGTGATTGGACCCCC |
| | GGAGCCCATCCTGGCCGTTGTGGGTGAGGACGCCGAGCTGCCCTGTCGCCTGTC |
| | TCCGAACGCGAGCGCCGAGCACTTGGAGCTACGCTGGTTCCGAAAGAAGGTTT |
| | CGCCGGCCGTGCTGGTGCATAGGGACGGGCGCGAGCAGGAAGCCGAGCAGATG |
| | CCCGAGTACCGCGGGCGGGCGACGCTGGTCCAGGACGGCATCGCCAAGGGGCG |
| | CGTGGCCTTGAGGATCCGTGGCGTCAGAGTCTCTGACGACGGGGAGTACACGT |
| | GCTTTTTCAGGGAGGATGGAAGCTACGAAGAAGCCCTGGTGCATCTGAAGGTG |
| | GCTGCTCTGGGCTCTGACCCTCACATCAGTATGCAAGTTCAAGAGAATGGAGAA |
| | ATCTGTCTGGAGTGCACCTCAGTGGGATGGTACCCAGAGCCCCAGGTGCAGTGG |
| | AGAACTTCCAAGGGAGAGAAGTTTCCATCTACATCAGAGTCCAGGAATCCTGAT |
| | GAAGAAGGTTTGTTCACTGTGGCTGCTTCAGTGATCATCAGAGACACTTCTGCG |
| | AAAAATGTGTCCTGCTACATCCAGAATCTCCTTCTTGGCCAGGAGAAGAAAGTA |
| | GAAATATCCATACCAGCTTCCTCCCTCCCAAGGCTGACTCCCTGGATAGTGGCT |
| | GTGGCTGTCATCCTGATGGTTCTAGGACTTCTCACCATTGGGTCCATATTTTTCA |
| | CTTGGAGACTATACAACGAAAGACCCAGAGAGAGGAGGAATGAATTCAGCTCT |
| | AAAGAGAGACTCCTGGAAGAACTCAAATGGAAAAAGGCTACCTTGCATGCAGT |
| | TGATGTGACTCTGGACCCAGACACAGCTCATCCCCACCTCTTTCTTTATGAGGA |
| | TTCAAAATCTGTTCGACTGGAAGATTCACGTCAGAAACTGCCTGAGAAAACAG |
| | AGAGATTTGACTCCTGGCCCTGTGTGTTGGGCCGTGAGACCTTCACCTCAGGAA |
| | GGCATTACTGGGAGGTGGAGGTGGGAGACAGGACTGACTGGGCAATCGGCGTG |
| | TGTAGGGAGAATGTGATGAAGAAAGGATTTGACCCCATGACTCCTGAGAATGG |
| | GTTCTGGGCTGTAGAGTTGTATGGAAATGGGTACTGGGCCCTCACTCCTCTCCG |
| | GACCCCTCTCCCATTGGCAGGGCCCCCACGCCGGGTTGGGATTTTCCTAGACTA |
| | TGAATCAGGAGACATCTCCTTCTACAACATGAATGATGGATCTGATATCTATAC |
| | TTTCTCCAATGTCACTTTCTCTGGCCCCCTCCGGCCCTTCTTTTGCCTATGGTCTA |
| | GCGGTAAAAAGCCCCTGACCATCTGCCCAATTGCTGATGGGCCTGAGAGGGTC |
| | ACAGTCATTGCTAATGCCCAGGACCTTTCTAAGGAGATCCCATTGTCCCCCATG |
| | GGGGAGGACTCTGCCCCTAGGGATGCAGACACTCTCCATTCTAAGCTAATCCCT |
| | ACCCAACCCAGCCAAGGGGCACCTTAAGGAATATCTCAGCTCATCTGTTTTCCT |
| | TTCCTCTAACCCCTCTCCTCCATAGCCTTCTGAGGCTTCACCTGCTAGCTTTACC |
| | CAGTCTGTTTCTTCCTGTTGGGTGGCAATTAATTAATCCTGTGAAGGTTACATTG |
| | CTGCTGCTAGAGAGGGTGGGGATTGCACCTTCCAAATCTGTTTCTGTACCAATA |
| | TTTGGGGGATGGAGGGGTGACTCAAACTGCTTCTAGTGTTCTCCTAATCCCTTA |
| | AGACTAGAACCTATAGGAAACTACTTGGAGCAAACTCAAAGGACAGATTAGGG |
| | ATCGAGATTGGGTCAGGTTAGCATGGGGTTGTGGTTGAAATATCTTGGTATCCA |
| | GGATAAGGGTATGTGGAAAAACAGGCTTTAGGCAAGTGGAAAATTCAAAATGT |
| | GCTGTGAAAGGACAATCTCAGGCTGAAATCCCATAAAGGAACTTGGAGGGAAT |
| | ATTATGATGGAGGGAAGTGAGGTGAATCCAGGCACATGATGAACACCTGGCTC |
| | ATCCATAGAGTTTTCACAGCCTATATCGCAAATTTTCTAAGCCACGTCCTATAG |
| | GACAGAGGAGACTGGCCCCACTTCTATGGGTCTGAGCTGTGGAAAAGGGAGAG |
| | CAGAGAGGAACTGAGATGAGCAGGGATGAAGGGTCAGGCAGAAAGCGTGATA |
| | GAGGAGAGAATTTTTGACAAAACTCAAAAGTTGTTTGCACAGCTGTTCTTTGTA |
| | CCCTGTTCCTTTCTCTGCGCCCTCCTGTTTCTCCCTTGCCTGGAAGTCATTCCACC |
| | CTCAATTTGTTGATCCACAAGTTTCCAGTTGTCCTCTTCTTTTTGTTATAGCATCT |
| | CTCTATTTCAAAGACATTCCTAGAAGTCATCCTTCAGTGATATCACCACTTGCTC |
| | AGTCACCATCTCAACCTTATGTCACCTCAGCCCTCATCTCAATGCCCAAACCCCT |
| | TACACACACCTTCAGTTAGCTTCAACTGCCTCCGTTTCCACACTGTGCACCTTTC |
| | ACTTTCCCTACCCAGCTTTCCTACATGCTGCCTCTCCTCAGGGTCCCCTGAATGC |
| | TGCATCATTGTGTTCAGTGCAGCTGGACTGATTGCACCTGTGTATTTGCCCCTGA |
| | GCACTTTCCTTTACACATGTGGCTTGTCTTGCCAATAGACTCCAGGCTTATACCT |
| | TCCATTTCCATCGTATTCTCCAGTTTCCAGGATAGACGTTGCTCATCGTCTTTAC |
| | CTAATAAATAAGTTTGTCTGATTGCTGAAA (SEQ ID NO: 75) |
| | >NP_001723.2 butyrophilin subfamily 1 member A1 precursor |
| | [Homo sapiens], amino acid sequence |
| | MAVFPSSGLPRCLLTLILLQLPKLDSAPFDVIGPPEPILAVVGEDAELPCRLSPNASAE |
| | HLELRWFRKKVSPAVLVHRDGREQEAEQMPEYRGRATLVQDGIAKGRVALRIRGV |
| | RVSDDGEYTCFFREDGSYEEALVHLKVAALGSDPHISMQVQENGEICLECTSVGW |
| | YPEPQVQWRTSKGEKFPSTSESRNPDEEGLFTVAASVIIRDTSAKNVSCYIQNLLLG |
| | QEKKVEISIPASSLPRLTPWIVAVAVILMVLGLLTIGSIFFTWRLYNERPRERRNEFSS |
| | KERLLEELKWKKATLHAVDVTLDPDTAHPHLFLYEDSKSVRLEDSRQKLPEKTERF |
| | DSWPCVLGRETFTSGRHYWEVEVGDRTDWAIGVCRENVMKKGFDPMTPENGFW |
| | AVELYGNGYWALTPLRTPLPLAGPPRRVGIFLDYESGDISFYNMNDGSDIYTFSNVT |
| | FSGPLRPFFCLWSSGKKPLTICPIADGPERVTVIANAQDLSKEIPLSPMGEDSAPRDA |
| | DTLHSKLIPTQPSQGAP |
| | (SEQ ID NO: 76) |
| Human | >NM_173799.4 Homo sapiens T cell immunoreceptor with Ig |
| TIGIT | and ITIM domains (TIGIT), mRNA, nucleic acid sequence |
| | ACATCTGCTTCCTGTAGGCCCTCTGGGCAGAAGCATGCGCTGGTGTCTCCTCCT |
| | GATCTGGGCCCAGGGGCTGAGGCAGGCTCCCCTCGCCTCAGGAATGATGACAG |
| | GCACAATAGAAACAACGGGGAACATTTCTGCAGAGAAAGGTGGCTCTATCATC |
| | TTACAATGTCACCTCTCCTCCACCACGGCACAAGTGACCCAGGTCAACTGGGAG |
| | CAGCAGGACCAGCTTCTGGCCATTTGTAATGCTGACTTGGGGTGGCACATCTCC |
| | CCATCCTTCAAGGATCGAGTGGCCCCAGGTCCCGGCCTGGGCCTCACCCTCCAG |
| | TCGCTGACCGTGAACGATACAGGGGAGTACTTCTGCATCTATCACACCTACCCT |
| | GATGGGACGTACACTGGGAGAATCTTCCTGGAGGTCCTAGAAAGCTCAGTGGC |
| | TGAGCACGGTGCCAGGTTCCAGATTCCATTGCTTGGAGCCATGGCCGCGACGCT |
| | GGTGGTCATCTGCACAGCAGTCATCGTGGTGGTCGCGTTGACTAGAAAGAAGA |
| | AAGCCCTCAGAATCCATTCTGTGGAAGGTGACCTCAGGAGAAAATCAGCTGGA |
| | CAGGAGGAATGGAGCCCCAGTGCTCCCTCACCCCCAGGAAGCTGTGTCCAGGC |
| | AGAAGCTGCACCTGCTGGGCTCTGTGGAGAGCAGCGGGGAGAGGACTGTGCCG |
| | AGCTGCATGACTACTTCAATGTCCTGAGTTACAGAAGCCTGGGTAACTGCAGCT |
| | TCTTCACAGAGACTGGTTAGCAACCAGAGGCATCTTCTGGAAGATACACTTTTG |
| | TCTTTGCTATTATAGATGAATATATAAGCAGCTGTACTCTCCATCAGTGCTGCGT |
| | GTGTGTGTGTGTGTGTATGTGTGTGTGTGTTCAGTTGAGTGAATAAATGTCATCC |
| | TCTTCTCCATCTTCATTTCCTTGGCCTTTTCGTTCTATTCCATTTTGCATTATGGC |
| | AGGCCTAGGGTGAGTAACGTGGATCTTGATCATAAATGCAAAATTAAAAAATA |
| | TCTTGACCTGGTTTTAAATCTGGCAGTTTGAGCAGATCCTATGTCTCTGAGAGAC |
| | ACATTCCTCATAATGGCCAGCATTTTGGGCTACAAGGTTTTGTGGTTGATGATG |
| | AGGATGGCATGACTGCAGAGCCATCCTCATCTCATTTTTTCACGTCATTTTCAGT |
| | AACTTTCACTCATTCAAAGGCAGGTTATAAGTAAGTCCTGGTAGCAGCCTCTAT |
| | GGGGAGATTTGAGAGTGACTAAATCTTGGTATCTGCCCTCAAGAACTTACAGTT |
| | AAATGGGGAGACAATGTTGTCATGAAAAGGTATTATAGTAAGGAGAGAAGGAG |
| | ACATACACAGGCCTTCAGGAAGAGACGACAGTTTGGGGTGAGGTAGTTGGCAT |
| | AGGCTTATCTGTGATGAAGTGGCCTGGGAGCACCAAGGGGATGTTGAGGCTAG |
| | TCTGGGAGGAGCAGGAGTTTTGTCTAGGGAACTTGTAGGAAATTCTTGGAGCTG |
| | AAAGTCCCACAAAGAAGGCCCTGGCACCAAGGGAGTCAGCAAACTTCAGATTT |
| | TATTCTCTGGGCAGGCATTTCAAGTTTCCTTTTGCTGTGACATACTCATCCATTA |
| | GACAGCCTGATACAGGCCTGTAGCCTCTTCCGGCCGTGTGTGCTGGGGAAGCCC |
| | CAGGAAACGCACATGCCCACACAGGGAGCCAAGTCGTAGCATTTGGGCCTTGA |
| | TCTACCTTTTCTGCATCAATACACTCTTGAGCCTTTGAAAAAAGAACGTTTCCCA |
| | CTAAAAAGAAAATGTGGATTTTTAAAATAGGGACTCTTCCTAGGGGAAAAAGG |
| | GGGGCTGGGAGTGATAGAGGGTTTAAAAAATAAACACCTTCAAACTAACTTCTT |
| | CGAACCCTTTTATTCACTCCCTGACGACTTTGTGCTGGGGTTGGGGTAACTGAA |
| | CCGCTTATTTCTGTTTAATTGCATTCAGGCTGGATCTTAGAAGACTTTTATCCTT |
| | CCACCATCTCTCTCAGAGGAATGAGCGGGGAGGTTGGATTTACTGGTGACTGAT |
| | TTTCTTTCATGGGCCAAGGAACTGAAAGAGAATGTGAAGCAAGGTTGTGTCTTG |
| | CGCATGGTTAAAAATAAAGCATTGTCCTGCTTCCTAAGACTTAGACTGGGGTTG |
| | ACAATTGTTTTAGCAACAAGACAATTCAACTATTTCTCCTAGGATTTTTATTATT |
| | ATTATTTTTTCACTTTTCTACCAAATGGGTTACATAGGAAGAATGAACTGAAAT |
| | CTGTCCAGAGCTCCAAGTCCTTTGGAAGAAAGATTAGATGAACGTAAAAATGTT |
| | GTTGTTTGCTGTGGCAGTTTACAGCATTTTTCTTGCAAAATTAGTGCAAATCTGT |
| | TGGAAATAGAACACAATTCACAAATTGGAAGTGAACTAAAATGTAATGACGAA |
| | AAGGGAGTAGTGTTTTGATTTGGAGGAGGTGTATATTCGGCAGAGGTTGGACTG |
| | AGAGTTGGGTGTTATTTAACATAATTATGGTAATTGGGAAACATTTATAAACAC |
| | TATTGGGATGGTGATAAAATACAAAAGGGCCTATAGATGTTAGAAATGGGTCA |
| | GGTTACTGAAATGGGATTCAATTTGAAAAAAATTTTTTTAAATAGAACTCACTG |
| | AACTAGATTCTCCTCTGAGAACCAGAGAAGACCATTTCATAGTTGGATTCCTGG |
| | AGACATGCGCTATCCACCACGTAGCCACTTTCCACATGTGGCCATCAACCACTT |
| | AAGATGGGGTTAGTTTAAATCAAGATGTGCTGTTATAATTGGTATAAGCATAAA |
| | ATCACACTAGATTCTGGAGATTTAATATGAATAATAAGAATACTATTTCAGTAG |
| | TTTTGGTATATTGTGTGTCAAAAATGATAATATTTTGGATGTATTGGGTGAAATA |
| | AAATATTAACATTA (SEQ ID NO: 77) |
| | >NP_776160.2 T-cell immunoreceptor with Ig and ITIM |
| | domains precursor [Homo sapiens], amino acid sequence |
| | MRWCLLLIWAQGLRQAPLASGMMTGTIETTGNISAEKGGSIILQCHLSSTTAQVTQ |
| | VNWEQQDQLLAICNADLGWHISPSFKDRVAPGPGLGLTLQSLTVNDTGEYFCIYHT |
| | YPDGTYTGRIFLEVLESSVAEHGARFQIPLLGAMAATLVVICTAVIVVVALTRKKK |
| | ALRIHSVEGDLRRKSAGQEEWSPSAPSPPGSCVQAEAAPAGLCGEQRGEDCAELHD |
| | YFNVLSYRSLGNCSFFTETG (SEQ ID NO: 78) |
| Human | >NM_001779.3 Homo sapiens CD58 molecule (CD58), |
| LFA-3 | transcript variant 1, mRNA, nucleic acid sequence |
| (CD58) | GAACTTAGGGCTGCTTGTGGCTGGGCACTCGCGCAGAGGCCGGCCCGACGAGC |
| | CATGGTTGCTGGGAGCGACGCGGGGCGGGCCCTGGGGGTCCTCAGCGTGGTCT |
| | GCCTGCTGCACTGCTTTGGTTTCATCAGCTGTTTTTCCCAACAAATATATGGTGT |
| | TGTGTATGGGAATGTAACTTTCCATGTACCAAGCAATGTGCCTTTAAAAGAGGT |
| | CCTATGGAAAAAACAAAAGGATAAAGTTGCAGAACTGGAAAATTCTGAATTCA |
| | GAGCTTTCTCATCTTTTAAAAATAGGGTTTATTTAGACACTGTGTCAGGTAGCCT |
| | CACTATCTACAACTTAACATCATCAGATGAAGATGAGTATGAAATGGAATCGCC |
| | AAATATTACTGATACCATGAAGTTCTTTCTTTATGTGCTTGAGTCTCTTCCATCT |
| | CCCACACTAACTTGTGCATTGACTAATGGAAGCATTGAAGTCCAATGCATGATA |
| | CCAGAGCATTACAACAGCCATCGAGGACTTATAATGTACTCATGGGATTGTCCT |
| | ATGGAGCAATGTAAACGTAACTCAACCAGTATATATTTTAAGATGGAAAATGAT |
| | CTTCCACAAAAAATACAGTGTACTCTTAGCAATCCATTATTTAATACAACATCA |
| | TCAATCATTTTGACAACCTGTATCCCAAGCAGCGGTCATTCAAGACACAGATAT |
| | GCACTTATACCCATACCATTAGCAGTAATTACAACATGTATTGTGCTGTATATG |
| | AATGGTATTCTGAAATGTGACAGAAAACCAGACAGAACCAACTCCAATTGATT |
| | GGTAACAGAAGATGAAGACAACAGCATAACTAAATTATTTTAAAAACTAAAAA |
| | GCCATCTGATTTCTCATTTGAGTATTACAATTTTTGAACAACTGTTGGAAATGTA |
| | ACTTGAAGCAGCTGCTTTAAGAAGAAATACCCACTAACAAAGAACAAGCATTA |
| | GTTTTGGCTGTCATCAACTTATTATATGACTAGGTGCTTGCTTTTTTTGTCAGTA |
| | AATTGTTTTTACTGATGATGTAGATACTTTTGTAAATAAATGTAAATATGTACAC |
| | AAGTGA (SEQ ID NO: 79) |
| | >NP_001770.1 lymphocyte function-associated antigen 3 |
| | isoform 1 [Homo sapiens], amino acid sequence |
| | MVAGSDAGRALGVLSVVCLLHCFGFISCFSQQIYGVVYGNVTFHVPSNVPLKEVL |
| | WKKQKDKVAELENSEFRAFSSFKNRVYLDTVSGSLTIYNLTSSDEDEYEMESPNIT |
| | DTMKFFLYVLESLPSPTLTCALTNGSIEVQCMIPEHYNSHRGLIMYSWDCPMEQCK |
| | RNSTSIYFKMENDLPQKIQCTLSNPLFNTTSSIILTTCIPSSGHSRHRYALIPI |
| | PLAVITTCIVLYMNGILKCDRKPDRTNSN (SEQ ID NO: 80) |
| Human | >NM_003037.5 Homo sapiens signaling lymphocytic activation |
| SLAM | molecule family member 1 (SLAMF1), transcript variant 1, |
| (CD150) | mRNA, nucleic acid sequence |
| | AGACAGCCTCTGCTGCATGACACGAAGCTTGCTTCTGCCTGGCATCTGTGAGCA |
| | GCTGCCAGGCTCCGGCCAGGATCCCTTCCTTCTCCTCATTGGCTGATGGATCCC |
| | AAGGGGCTCCTCTCCTTGACCTTCGTGCTGTTTCTCTCCCTGGCTTTTGGGGCAA |
| | GCTACGGAACAGGTGGGCGCATGATGAACTGCCCAAAGATTCTCCGGCAGTTG |
| | GGAAGCAAAGTGCTGCTGCCCCTGACATATGAAAGGATAAATAAGAGCATGAA |
| | CAAAAGCATCCACATTGTCGTCACAATGGCAAAATCACTGGAGAACAGTGTCG |
| | AGAACAAAATAGTGTCTCTTGATCCATCCGAAGCAGGCCCTCCACGTTATCTAG |
| | GAGATCGCTACAAGTTTTATCTGGAGAATCTCACCCTGGGGATACGGGAAAGC |
| | AGGAAGGAGGATGAGGGATGGTACCTTATGACCCTGGAGAAAAATGTTTCAGT |
| | TCAGCGCTTTTGCCTGCAGTTGAGGCTTTATGAGCAGGTCTCCACTCCAGAAAT |
| | TAAAGTTTTAAACAAGACCCAGGAGAACGGGACCTGCACCTTGATACTGGGCT |
| | GCACAGTGGAGAAGGGGGACCATGTGGCTTACAGCTGGAGTGAAAAGGCGGGC |
| | ACCCACCCACTGAACCCAGCCAACAGCTCCCACCTCCTGTCCCTCACCCTCGGC |
| | CCCCAGCATGCTGACAATATCTACATCTGCACCGTGAGCAACCCTATCAGCAAC |
| | AATTCCCAGACCTTCAGCCCGTGGCCCGGATGCAGGACAGACCCCTCAGAAAC |
| | AAAACCATGGGCAGTGTATGCTGGGCTGTTAGGGGGTGTCATCATGATTCTCAT |
| | CATGGTGGTAATACTACAGTTGAGAAGAAGAGGTAAAACGAACCATTACCAGA |
| | CAACAGTGGAAAAAAAAAGCCTTACGATCTATGCCCAAGTCCAGAAACCAGGT |
| | CCTCTTCAGAAGAAACTTGACTCCTTCCCAGCTCAGGACCCTTGCACCACCATA |
| | TATGTTGCTGCCACAGAGCCTGTCCCAGAGTCTGTCCAGGAAACAAATTCCATC |
| | ACAGTCTATGCTAGTGTGACACTTCCAGAGAGCTGACACCAGAGACCAACAAA |
| | GGGACTTTCTGAAGGAAAATGGAAAAACCAAAATGAACACTGAACTTGGCCAC |
| | AGGCCCCAAGTTTCCTCTGGCAGACATGCTGCACGTCTGTACCCTTCTCAGATC |
| | AACTCCCTGGTGATGTTTCTTCCACATACATCTGTGAAATGAACAAGGAAGTGA |
| | GGCTTCCCAAGAATTTAGCTTGCTGTGCAGTGGCTGCAGGCGCAGAACAGAGC |
| | GTTACTTGATAACAGCGTTCCATCTTTGTGTTGTAGCAGATGAAATGGACAGTA |
| | ATGTGAGTTCAGACTTTGGGCATCTTGCTCTTGGCTGGAACTGGATAATAAAAA |
| | TCAGACTGAAAGCCAGGACATCTGAGTACCTATCTCACACACTGGACCACCAGT |
| | CACAAAGTCTGGAAAAGTTTACATTTTGGCTATCTTTACTTTGTTCTGGGAGCTG |
| | ATCATGATAACCTGCAGACCTGATCAAGCCTCTGTGCCTCAGTTTCTCTCTCAG |
| | GATAAAGAGTGAATAGAGGCTGAAGGGTGAATTTCTTATTATACATAAAACAC |
| | TCTGATATTATTGTATAAAGGAAGCTAAGAATATTATTTTATTTGCAAAACCCA |
| | GAAGCTAAAAAGTCAATAAACAGAAAGAATGATTTTGAGATCTCTGAGTTTTG |
| | AACAGTGGACTGGAAACCATGTAAGAGCCTTAAAAGTACAGTTCTGTGCAAAT |
| | GGCATTCAGTTTTAAAGAAAAACGTAGCAAATGTTTGATGGTGCTGTTACAAAG |
| | GAGCTTGGAATACTCAGAGGAACTTGTCCCATGGTGATTTTTCACTTCTCAAAA |
| | TGATGTTTAAATCCCAGTTCTCTGTTGATTCCCTTGAACAACAAACCTGGAACCT |
| | CAGCTAAGACTCTCTGTGACCAGATTCTGAACCTCTTATATCCAGGGCTTCAAG |
| | GGGTATTGCAGGTCAAGGTCTTTCCTAGGCACTTTCTACTCCCTGCATACCTCTC |
| | CTCACACTAAATTTATCCTCTAGTAGAAAATTAAGTTATTTTGGTCTAACAGCTT |
| | CAAATCTTTGAATGCTCAATAACTTATTTTGCAAGCTGCAGGCAGAAAGAGACT |
| | TTTTAAGTAAAGTCCTTTGTTTTTTCCTATTCTCTGCTTTTAGACAGGCTGTCCTC |
| | AATTTAAGCCCTGCTTTTTCTTATTGTTTCTTATATAAACTTGGTAAGTACTGTA |
| | AGAAACAGCCACTATCATACCATTGCATAATAAGGAGCACCAACTTCCCAGCTC |
| | AAAACTCAGGTCCTTATTGCCTTGTATCTTACCTCCTCTATGAGGTCAATTCACA |
| | TTGTAAGCCTGTTGCTTAGTGCATCTCGTTTCCTGGTACCAGCTTCTTTAATAGA |
| | GTTCTTAGTTGCAATCAACAGAAGCTGGCTTTGGCTTTTTTATGTAGAAAAGGA |
| | ACCTATTGAAAAGATACTGATTGGTTCCAATAACTGCTAGAAGTTTCTGCAAAA |
| | CCATGCTTTGAAAGTGAGCAGGAAAAGAAGAGACTAGGCTGTGGCTGGGAGCA |
| | CAGCCAAAATTACAAAACCAGCCCAGGGATGATGATCCTGTTCATGCACAGCC |
| | ACTGTCCCCAGCACTAGGCACAGACTCTACCACTGCCTCACTGTCTCTGCTGGA |
| | CTTGGAAACTTGATATTACTGTTACTGCTGCACTGTCTGCCATGAAAATGAATTC |
| | TCCAGGGTCCCTTCTTCATCCTTTCATCTCTAGCTTATAATTCAAAGTCTGGGAT |
| | TGAGTGGCCAATCCTAGGTCACATGTCCATGTCCTATCTCCAAGGGGGGCTGGG |
| | AATTGAATATCTGGCATTTTCCACTTTCACTTCTTATGAATTAAGGAATTCTACA |
| | AATAATAGAAGTGGGATTCAGGTGGTAGGCAGACAAAAAAGCCTCACAATTAT |
| | CCACTACGCCACCCTTGTATAACCTTACCCTCATTCACTGTCTACTCTCAAAACT |
| | GTGGAGCTACTAATGAAGATTTGTAAACCCGGGCTTATGAGCACCCATTCCTTT |
| | ACTACAACTCAGATTGCTCTAGAAGCTCAGTTCCCAGCACTTGGATTTTTCCAGT |
| | AGCTGAATTCTACCTGAAGGAAGGGCAGAAACAAAGGGTGAAGAAGAGGCTAT |
| | CACTTCCAAGTATCCTGCACCCCTGGGCTCAAGACCTCACTGGGGAGGGAGTCT |
| | TTTGGGCCACCCACCAAACAGCACTGGCATTATGCCTCTCACCCTAGACCATGG |
| | TTACACGTGGTAAAACAACCCCTTCTGGTGATACATTCACAACTCTCTAGTTTCC |
| | CCCAAATGGCACTATGGGGAGCGGGAGCTTGCCTTTTCCTCAGACTTAAAACAA |
| | TAAGTTTTCCCCGTGTTTCCCCTCTAATGCTGTTTTCTTTTGACCAAGCATGTCTG |
| | AATTCTAGAGAAGTCAGGAGGAACACACCCATTCTCGGTTTGAAGGGACTGAT |
| | GTTCTGAAGTACAACTGGGCACAGTCCCAGGCTCTTCAGGACGCTTCCTCCATT |
| | CACACAGCGGGGATGTGATTGTTACAGCGGGTGGTGTGTGCTGGCTGAGAAGC |
| | CACTGTGAATTGATTCTTCTTCTGAAGTTTATGTTTCTACTTTTTGGAAATGAAT |
| | AAATTACAGCCAGTCCATCAAGGAAA (SEQ ID NO: 81) |
| | >NP_003028.1 signaling lymphocytic activation molecule |
| | isoform b precursor [Homo sapiens], amino acid sequence |
| | MDPKGLLSLTFVLFLSLAFGASYGTGGRMMNCPKILRQLGSKVLLPLTYERINKSM |
| | NKSIHIVVTMAKSLENSVENKIVSLDPSEAGPPRYLGDRYKFYLENLTLGIRESRKE |
| | DEGWYLMTLEKNVSVQRFCLQLRLYEQVSTPEIKVLNKTQENGTCTLILGCTVEKG |
| | DHVAYSWSEKAGTHPLNPANSSHLLSLTLGPQHADNIYICTVSNPISNNSQTFSPWP |
| | GCRTDPSETKPWAVYAGLLGGVIMILIMVVILQLRRRGKTNHYQTTVEKKSLTIYA |
| | QVQKPGPLQKKLDSFPAQDPCTTIYVAATEPVPESVQETNSITVYASVTLPES |
| | (SEQ ID NO: 82) |
| Human CD28 | >NM_006139.4 Homo sapiens CD28 molecule (CD28), |
| | transcript variant 1, mRNA, nucleic acid sequence |
| | ACACTTCGGGTTCCTCGGGGAGGAGGGGCTGGAACCCTAGCCCATCGTCAGGA |
| | CAAAGATGCTCAGGCTGCTCTTGGCTCTCAACTTATTCCCTTCAATTCAAGTAAC |
| | AGGAAACAAGATTTTGGTGAAGCAGTCGCCCATGCTTGTAGCGTACGACAATG |
| | CGGTCAACCTTAGCTGCAAGTATTCCTACAATCTCTTCTCAAGGGAGTTCCGGG |
| | CATCCCTTCACAAAGGACTGGATAGTGCTGTGGAAGTCTGTGTTGTATATGGGA |
| | ATTACTCCCAGCAGCTTCAGGTTTACTCAAAAACGGGGTTCAACTGTGATGGGA |
| | AATTGGGCAATGAATCAGTGACATTCTACCTCCAGAATTTGTATGTTAACCAAA |
| | CAGATATTTACTTCTGCAAAATTGAAGTTATGTATCCTCCTCCTTACCTAGACAA |
| | TGAGAAGAGCAATGGAACCATTATCCATGTGAAAGGGAAACACCTTTGTCCAA |
| | GTCCCCTATTTCCCGGACCTTCTAAGCCCTTTTGGGTGCTGGTGGTGGTTGGTGG |
| | AGTCCTGGCTTGCTATAGCTTGCTAGTAACAGTGGCCTTTATTATTTTCTGGGTG |
| | AGGAGTAAGAGGAGCAGGCTCCTGCACAGTGACTACATGAACATGACTCCCCG |
| | CCGCCCCGGGCCCACCCGCAAGCATTACCAGCCCTATGCCCCACCACGCGACTT |
| | CGCAGCCTATCGCTCCTGACACGGACGCCTATCCAGAAGCCAGCCGGCTGGCA |
| | GCCCCCATCTGCTCAATATCACTGCTCTGGATAGGAAATGACCGCCATCTCCAG |
| | CCGGCCACCTCAGGCCCCTGTTGGGCCACCAATGCCAATTTTTCTCGAGTGACT |
| | AGACCAAATATCAAGATCATTTTGAGACTCTGAAATGAAGTAAAAGAGATTTCC |
| | TGTGACAGGCCAAGTCTTACAGTGCCATGGCCCACATTCCAACTTACCATGTAC |
| | TTAGTGACTTGACTGAGAAGTTAGGGTAGAAAACAAAAAGGGAGTGGATTCTG |
| | GGAGCCTCTTCCCTTTCTCACTCACCTGCACATCTCAGTCAAGCAAAGTGTGGT |
| | ATCCACAGACATTTTAGTTGCAGAAGAAAGGCTAGGAAATCATTCCTTTTGGTT |
| | AAATGGGTGTTTAATCTTTTGGTTAGTGGGTTAAACGGGGTAAGTTAGAGTAGG |
| | GGGAGGGATAGGAAGACATATTTAAAAACCATTAAAACACTGTCTCCCACTCA |
| | TGAAATGAGCCACGTAGTTCCTATTTAATGCTGTTTTCCTTTAGTTTAGAAATAC |
| | ATAGACATTGTCTTTTATGAATTCTGATCATATTTAGTCATTTTGACCAAATGAG |
| | GGATTTGGTCAAATGAGGGATTCCCTCAAAGCAATATCAGGTAAACCAAGTTGC |
| | TTTCCTCACTCCCTGTCATGAGACTTCAGTGTTAATGTTCACAATATACTTTCGA |
| | AAGAATAAAATAGTTCTCCTACATGAAGAAAGAATATGTCAGGAAATAAGGTC |
| | ACTTTATGTCAAAATTATTTGAGTACTATGGGACCTGGCGCAGTGGCTCATGCT |
| | TGTAATCCCAGCACTTTGGGAGGCCGAGGTGGGCAGATCACTTGAGATCAGGA |
| | CCAGCCTGGTCAAGATGGTGAAACTCCGTCTGTACTAAAAATACAAAATTTAGC |
| | TTGGCCTGGTGGCAGGCACCTGTAATCCCAGCTGCCCAAGAGGCTGAGGCATG |
| | AGAATCGCTTGAACCTGGCAGGCGGAGGTTGCAGTGAGCCGAGATAGTGCCAC |
| | AGCTCTCCAGCCTGGGCGACAGAGTGAGACTCCATCTCAAACAACAACAACAA |
| | CAACAACAACAACAACAAACCACAAAATTATTTGAGTACTGTGAAGGATTATTT |
| | GTCTAACAGTTCATTCCAATCAGACCAGGTAGGAGCTTTCCTGTTTCATATGTTT |
| | CAGGGTTGCACAGTTGGTCTCTTTAATGTCGGTGTGGAGATCCAAAGTGGGTTG |
| | TGGAAAGAGCGTCCATAGGAGAAGTGAGAATACTGTGAAAAAGGGATGTTAGC |
| | ATTCATTAGAGTATGAGGATGAGTCCCAAGAAGGTTCTTTGGAAGGAGGACGA |
| | ATAGAATGGAGTAATGAAATTCTTGCCATGTGCTGAGGAGATAGCCAGCATTA |
| | GGTGACAATCTTCCAGAAGTGGTCAGGCAGAAGGTGCCCTGGTGAGAGCTCCTT |
| | TACAGGGACTTTATGTGGTTTAGGGCTCAGAGCTCCAAAACTCTGGGCTCAGCT |
| | GCTCCTGTACCTTGGAGGTCCATTCACATGGGAAAGTATTTTGGAATGTGTCTTT |
| | TGAAGAGAGCATCAGAGTTCTTAAGGGACTGGGTAAGGCCTGACCCTGAAATG |
| | ACCATGGATATTTTTCTACCTACAGTTTGAGTCAACTAGAATATGCCTGGGGAC |
| | CTTGAAGAATGGCCCTTCAGTGGCCCTCACCATTTGTTCATGCTTCAGTTAATTC |
| | AGGTGTTGAAGGAGCTTAGGTTTTAGAGGCACGTAGACTTGGTTCAAGTCTCGT |
| | TAGTAGTTGAATAGCCTCAGGCAAGTCACTGCCCACCTAAGATGATGGTTCTTC |
| | AACTATAAAATGGAGATAATGGTTACAAATGTCTCTTCCTATAGTATAATCTCC |
| | ATAAGGGCATGGCCCAAGTCTGTCTTTGACTCTGCCTATCCCTGACATTTAGTA |
| | GCATGCCCGACATACAATGTTAGCTATTGGTATTATTGCCATATAGATAAATTA |
| | TGTATAAAAATTAAACTGGGCAATAGCCTAAGAAGGGGGGAATATTGTAACAC |
| | AAATTTAAACCCACTACGCAGGGATGAGGTGCTATAATATGAGGACCTTTTAAC |
| | TTCCATCATTTTCCTGTTTCTTGAAATAGTTTATCTTGTAATGAAATATAAGGCA |
| | CCTCCCACTTTTATGTATAGAAAGAGGTCTTTTAATTTTTTTTTAATGTGAGAAG |
| | GAAGGGAGGAGTAGGAATCTTGAGATTCCAGATCGAAAATACTGTACTTTGGTT |
| | GATTTTTAAGTGGGCTTCCATTCCATGGATTTAATCAGTCCCAAGAAGATCAAA |
| | CTCAGCAGTACTTGGGTGCTGAAGAACTGTTGGATTTACCCTGGCACGTGTGCC |
| | ACTTGCCAGCTTCTTGGGCACACAGAGTTCTTCAATCCAAGTTATCAGATTGTAT |
| | TTGAAAATGACAGAGCTGGAGAGTTTTTTGAAATGGCAGTGGCAAATAAATAA |
| | ATACTTTTTTTTAAATGGAAAGACTTGATCTATGGTAATAAATGATTTTGTTTTC |
| | TGACTGGAAAAATAGGCCTACTAAAGATGAATCACACTTGAGATGTTTCTTACT |
| | CACTCTGCACAGAAACAAAGAAGAAATGTTATACAGGGAAGTCCGTTTTCACT |
| | ATTAGTATGAACCAAGAAATGGTTCAAAAACAGTGGTAGGAGCAATGCTTTCA |
| | TAGTTTCAGATATGGTAGTTATGAAGAAAACAATGTCATTTGCTGCTATTATTGT |
| | AAGAGTCTTATAATTAATGGTACTCCTATAATTTTTGATTGTGAGCTCACCTATT |
| | TGGGTTAAGCATGCCAATTTAAAGAGACCAAGTGTATGTACATTATGTTCTACA |
| | TATTCAGTGATAAAATTACTAAACTACTATATGTCTGCTTTAAATTTGTACTTTA |
| | ATATTGTCTTTTGGTATTAAGAAAGATATGCTTTCAGAATAGATATGCTTCGCTT |
| | TGGCAAGGAATTTGGATAGAACTTGCTATTTAAAAGAGGTGTGGGGTAAATCCT |
| | TGTATAAATCTCCAGTTTAGCCTTTTTTGAAAAAGCTAGACTTTCAAATACTAAT |
| | TTCACTTCAAGCAGGGTACGTTTCTGGTTTGTTTGCTTGACTTCAGTCACAATTT |
| | CTTATCAGACCAATGGCTGACCTCTTTGAGATGTCAGGCTAGGCTTACCTATGT |
| | GTTCTGTGTCATGTGAATGCTGAGAAGTTTGACAGAGATCCAACTTCAGCCTTG |
| | ACCCCATCAGTCCCTCGGGTTAACTAACTGAGCCACCGGTCCTCATGGCTATTT |
| | TAATGAGGGTATTGATGGTTAAATGCATGTCTGATCCCTTATCCCAGCCATTTGC |
| | ACTGCCAGCTGGGAACTATACCAGACCTGGATACTGATCCCAAAGTGTTAAATT |
| | CAACTACATGCTGGAGATTAGAGATGGTGCCAATAAAGGACCCAGAACCAGGA |
| | TCTTGATTGCTATAGACTTATTAATAATCCAGGTCAAAGAGAGTGACACACACT |
| | CTCTCAAGACCTGGGGTGAGGGAGTCTGTGTTATCTGCAAGGCCATTTGAGGCT |
| | CAGAAAGTCTCTCTTTCCTATAGATATATGCATACTTTCTGACATATAGGAATGT |
| | ATCAGGAATACTCAACCATCACAGGCATGTTCCTACCTCAGGGCCTTTACATGT |
| | CCTGTTTACTCTGTCTAGAATGTCCTTCTGTAGATGACCTGGCTTGCCTCGTCAC |
| | CCTTCAGGTCCTTGCTCAAGTGTCATCTTCTCCCCTAGTTAAACTACCCCACACC |
| | CTGTCTGCTTTCCTTGCTTATTTTTCTCCATAGCATTTTACCATCTCTTACATTAG |
| | ACATTTTTCTTATTTATTTGTAGTTTATAAGCTTCATGAGGCAAGTAACTTTGCT |
| | TTGTTTCTTGCTGTATCTCCAGTGCCCAGAGCAGTGCCTGGTATATAATAAATAT |
| | TTATTGACTGAGTGAA (SEQ ID NO: 83) |
| | >NP_006130.1 T-cell-specific surface glycoprotein CD28 |
| | isoform 1 precursor [Homo sapiens], amino acid sequence |
| | MLRLLLALNLFPSIQVTGNKILVKQSPMLVAYDNAVNLSCKYSYNLFSREFRASLH |
| | KGLDSAVEVCVVYGNYSQQLQVYSKTGFNCDGKLGNESVTFYLQNLYVNQTDIYF |
| | CKIEVMYPPPYLDNEKSNGTIIHVKGKHLCPSPLFPGPSKPFWVLVVVGGVLACYSL |
| | LVTVAFIIFWVRSKRSRLLHSDYMNMTPRRPGPTRKHYQPYAPPRDFAAYRS |
| | (SEQ ID NO: 84) |
| Human | >NM_144615.2 Homo sapiens transmembrane and immunoglobulin |
| CD28H | domain containing 2 (TMIGD2), transcript variant 1, mRNA, |
| | nucleic acid sequence |
| | GGAAGTCTGTCAACTGGGAGGGGGAGAGGGGGGTGATGGGCCAGGAATGGGG |
| | TCCCCGGGCATGGTGCTGGGCCTCCTGGTGCAGATCTGGGCCCTGCAAGAAGCC |
| | TCAAGCCTGAGCGTGCAGCAGGGGCCCAACTTGCTGCAGGTGAGGCAGGGCAG |
| | TCAGGCGACCCTGGTCTGCCAGGTGGACCAGGCCACAGCCTGGGAACGGCTCC |
| | GTGTTAAGTGGACAAAGGATGGGGCCATCCTGTGTCAACCGTACATCACCAAC |
| | GGCAGCCTCAGCCTGGGGGTCTGCGGGCCCCAGGGACGGCTCTCCTGGCAGGC |
| | ACCCAGCCATCTCACCCTGCAGCTGGACCCTGTGAGCCTCAACCACAGCGGGGC |
| | GTACGTGTGCTGGGCGGCCGTAGAGATTCCTGAGTTGGAGGAGGCTGAGGGCA |
| | ACATAACAAGGCTCTTTGTGGACCCAGATGACCCCACACAGAACAGAAACCGG |
| | ATCGCAAGCTTCCCAGGATTCCTCTTCGTGCTGCTGGGGGTGGGAAGCATGGGT |
| | GTGGCTGCGATCGTGTGGGGTGCCTGGTTCTGGGGCCGCCGCAGCTGCCAGCAA |
| | AGGGACTCAGGTAACAGCCCAGGAAATGCATTCTACAGCAACGTCCTATACCG |
| | GCCCCGGGGGGCCCCAAAGAAGAGTGAGGACTGCTCTGGAGAGGGGAAGGAC |
| | CAGAGGGGCCAGAGCATTTATTCAACCTCCTTCCCGCAACCGGCCCCCCGCCAG |
| | CCGCACCTGGCGTCAAGACCCTGCCCCAGCCCGAGACCCTGCCCCAGCCCCAGG |
| | CCCGGCCACCCCGTCTCTATGGTCAGGGTCTCTCCTAGACCAAGCCCCACCCAG |
| | CAGCCGAGGCCAAAAGGGTTCCCCAAAGTGGGAGAGGAGTGAGAGATCCCAG |
| | GAGACCTCAACAGGACCCCACCCATAGGTACACACAAAAAAGGGGGGATCGAG |
| | GCCAGACACGGTGGCTCACGCCTGTAATCCCAGCAGTTTGGGAAGCCGAGGCG |
| | GGTGGAACACTTGAGGTCAGGGGTTTGAGACCAGCCTGGCTTGAACCTGGGAG |
| | GCGGAGGTTGCAGTGAGCCGAGATTGCGCCACTGCACTCCAGCCTGGGCGACA |
| | GAGTGAGACTCCGTCTCAAAAAAAACAAAAAGCAGGAGGATTGGGAGCCTGTC |
| | AGCCCCATCCTGAGACCCCGTCCTCATTTCTGTAATGATGGATCTCGCTCCCACT |
| | TTCCCCCAAGAACCTAATAAAGGCTTGTGAAGAAAAAGCAAAAAAAAAAAAAA |
| | AAAA (SEQ ID NO: 85) |
| | >NP_653216.2 transmembrane and immunoglobulin domain- |
| | containing protein 2 isoform 1 precursor [Homo sapiens], |
| | amino acid sequence |
| | MGSPGMVLGLLVQIWALQEASSLSVQQGPNLLQVRQGSQATLVCQVDQATAWER |
| | LRVKWTKDGAILCQPYITNGSLSLGVCGPQGRLSWQAPSHLTLQLDPVSLNHSGAY |
| | VCWAAVEIPELEEAEGNITRLFVDPDDPTQNRNRIASFPGFLFVLLGVGSMGVAAIV |
| | WGAWFWGRRSCQQRDSGNSPGNAFYSNVLYRPRGAPKKSEDCSGEGKDQRGQSI |
| | YSTSFPQPAPRQPHLASRPCPSPRPCPSPRPGHPVSMVRVSPRPSPTQQPRPKGFPKV |
| | GEE (SEQ ID NO: 86) |
| Human CD2 | >NM_001328609.2 Homo sapiens CD2 molecule (CD2), |
| | transcript variant 1, mRNA, nucleic acid sequence |
| | AGTCTCACTTCAGTTCCTTTTGCATGAAGAGCTCAGAATCAAAAGAGGAAACCA |
| | ACCCCTAAGATGAGCTTTCCATGTAAATTTGTAGCCAGCTTCCTTCTGATTTTCA |
| | ATGTTTCTTCCAAAGGTGCAGTCTCCAAAGAGATTACGAATGCCTTGGAAACCT |
| | GGGGTGCCTTGGGTCAGGACATCAACTTGGACATTCCTAGTTTTCAAATGAGTG |
| | ATGATATTGACGATATAAAATGGGAAAAAACTTCAGACAAGAAAAAGATTGCA |
| | CAATTCAGAAAAGAGAAAGAGACTTTCAAGGAAAAAGATACATATAAGCTATT |
| | TAAAAATGGAACTCTGAAAATTAAGCATCTGAAGACCGATGATCAGGATATCT |
| | ACAAGGTATCAATATATGATACAAAAGGAAAAAATGTGTTGGAAAAAATATTT |
| | GATTTGAAGATTCAAGAGAGGGTCTCAAAACCAAAGATCTCCTGGACTTGTATC |
| | AACACAACCCTGACCTGTGAGGTAATGAATGGAACTGACCCCGAATTAAACCT |
| | GTATCAAGATGGGAAACATCTAAAACTTTCTCAGAGGGTCATCACACACAAGT |
| | GGACCACCAGCCTGAGTGCAAAATTCAAGTGCACAGCAGGGAACAAAGTCAGC |
| | AAGGAATCCAGTGTCGAGCCTGTCAGCTGTCCAGGAGGCAGCATCCTTGGCCA |
| | GAGTAATGGGCTCTCTGCCTGGACCCCTCCCAGCCATCCCACTTCTCTTCCTTTT |
| | GCAGAGAAAGGTCTGGACATCTATCTCATCATTGGCATATGTGGAGGAGGCAG |
| | CCTCTTGATGGTCTTTGTGGCACTGCTCGTTTTCTATATCACCAAAAGGAAAAA |
| | ACAGAGGAGTCGGAGAAATGATGAGGAGCTGGAGACAAGAGCCCACAGAGTA |
| | GCTACTGAAGAAAGGGGCCGGAAGCCCCACCAAATTCCAGCTTCAACCCCTCA |
| | GAATCCAGCAACTTCCCAACATCCTCCTCCACCACCTGGTCATCGTTCCCAGGC |
| | ACCTAGTCATCGTCCCCCGCCTCCTGGACACCGTGTTCAGCACCAGCCTCAGAA |
| | GAGGCCTCCTGCTCCGTCGGGCACACAAGTTCACCAGCAGAAAGGCCCGCCCCT |
| | CCCCAGACCTCGAGTTCAGCCAAAACCTCCCCATGGGGCAGCAGAAAACTCATT |
| | GTCCCCTTCCTCTAATTAAAAAAGATAGAAACTGTCTTTTTCAATAAAAAGCAC |
| | TGTGGATTTCTGCCCTCCTGATGTGCATATCCGTACTTCCATGAGGTGTTTTCTG |
| | TGTGCAGAACATTGTCACCTCCTGAGGCTGTGGGCCACAGCCACCTCTGCATCT |
| | TCGAACTCAGCCATGTGGTCAACATCTGGAGTTTTTGGTCTCCTCAGAGAGCTC |
| | CATCACACCAGTAAGGAGAAGCAATATAAGTGTGATTGCAAGAATGGTAGAGG |
| | ACCGAGCACAGAAATCTTAGAGATTTCTTGTCCCCTCTCAGGTCATGTGTAGAT |
| | GCGATAAATCAAGTGATTGGTGTGCCTGGGTCTCACTACAAGCAGCCTATCTGC |
| | TTAAGAGACTCTGGAGTTTCTTATGTGCCCTGGTGGACACTTGCCCACCATCCTG |
| | TGAGTAAAAGTGAAATAAAAGCTTTGACTAGA (SEQ ID NO: 87) |
| | >NP_001315538.1 T-cell surface antigen CD2 isoform 1 |
| | precursor [Homo sapiens], amino acid sequence |
| | MSFPCKFVASFLLIFNVSSKGAVSKEITNALETWGALGQDINLDIPSFQMSDDIDDIK |
| | WEKTSDKKKIAQFRKEKETFKEKDTYKLFKNGTLKIKHLKTDDQDIYKVSIYDTKG |
| | KNVLEKIFDLKIQERVSKPKISWTCINTTLTCEVMNGTDPELNLYQDGKHLKLSQR |
| | VITHKWTTSLSAKFKCTAGNKVSKESSVEPVSCPGGSILGQSNGLSAWTPPSHPTSL |
| | PFAEKGLDIYLIIGICGGGSLLMVFVALLVFYITKRKKQRSRRNDEELETRAHRVAT |
| | EERGRKPHQIPASTPQNPATSQHPPPPPGHRSQAPSHRPPPPGHRVQHQPQKRPPAPS |
| | GTQVHQQKGPPLPRPRVQPKPPHGAAENSLSPSSN (SEQ ID NO: 88) |
| Human CD48 | >NM_001778.4 Homo sapiens CD48 molecule (CD48), |
| | transcript variant 1, mRNA, nucleic acid sequence |
| | CTTTTTCTAGCCAGGCTCTCAACTGTCTCCTGCGTTGCTGGGAAGTTCTGGAAGG |
| | AAGCATGTGCTCCAGAGGTTGGGATTCGTGTCTGGCTCTGGAATTGCTACTGCT |
| | GCCTCTGTCACTCCTGGTGACCAGCATTCAAGGTCACTTGGTACATATGACCGT |
| | GGTCTCCGGCAGCAACGTGACTCTGAACATCTCTGAGAGCCTGCCTGAGAACTA |
| | CAAACAACTAACCTGGTTTTATACTTTCGACCAGAAGATTGTAGAATGGGATTC |
| | CAGAAAATCTAAGTACTTTGAATCCAAATTTAAAGGCAGGGTCAGACTTGATCC |
| | TCAGAGTGGCGCACTGTACATCTCTAAGGTCCAGAAAGAGGACAACAGCACCT |
| | ACATCATGAGGGTGTTGAAAAAGACTGGGAATGAGCAAGAATGGAAGATCAAG |
| | CTGCAAGTGCTTGACCCTGTACCCAAGCCTGTCATCAAAATTGAGAAGATAGAA |
| | GACATGGATGACAACTGTTATCTGAAACTGTCATGTGTGATACCTGGCGAGTCT |
| | GTAAACTACACCTGGTATGGGGACAAAAGGCCCTTCCCAAAGGAGCTCCAGAA |
| | CAGTGTGCTTGAAACCACCCTTATGCCACATAATTACTCCAGGTGTTATACTTGC |
| | CAAGTCAGCAATTCTGTGAGCAGCAAGAATGGCACGGTCTGCCTCAGTCCACCC |
| | TGTACCCTGGCCCGGTCCTTTGGAGTAGAATGGATTGCAAGTTGGCTAGTGGTC |
| | ACGGTGCCCACCATTCTTGGCCTGTTACTTACCTGAGATGAGCTCTTTTAACTCA |
| | AGCGAAACTTCAAGGCCAGAAGATCTTGCCTGTTGGTGATCATGCTCCTCACCA |
| | GGACAGAGACTGTATAGGCTGACCAGAAGCATGCTGCTGAATTATCAACGAGG |
| | ATTTTCAAGTTAACTTTTAAATACTGGTTATTATTTAATTTTATATCCCTTTGTTG |
| | TTTTCTAGTACACAGAGATATAGAGATACACATGCTTTTTTCCCACCCAAAATT |
| | GTGACAACATTATGTGAATGTTTTATTATTTTTTAAAATAAACATTTGATATAAT |
| | TGTCAATTAACTGAA (SEQ ID NO: 89) |
| | >NP_001769.2 CD48 antigen isoform 1 precursor [Homo |
| | sapiens], amino acid sequence |
| | MCSRGWDSCLALELLLLPLSLLVTSIQGHLVHMTVVSGSNVTLNISESLPENYKQLT |
| | WFYTFDQKIVEWDSRKSKYFESKFKGRVRLDPQSGALYISKVQKEDNSTYIMRVLK |
| | KTGNEQEWKIKLQVLDPVPKPVIKIEKIEDMDDNCYLKLSCVIPGESVNYTWYGDK |
| | RPFPKELQNSVLETTLMPHNYSRCYTCQVSNSVSSKNGTVCLSPPCTLARSFGVEWI |
| | ASWLVVTVPTILGLLLT (SEQ ID NO: 90) |
| Human | >NM_006566.4 Homo sapiens CD226 molecule (CD226), |
| CD226 | transcript variant 1, mRNA, nucleic acid sequence |
| | GCAGATGGGAAGAAGCGTTAGAGCGAGCAGCACTCACATCTCAAGAACCAGCC |
| | TTTCAAACAGTTTCCAGAGATGGATTATCCTACTTTACTTTTGGCTCTTCTTCAT |
| | GTATACAGAGCTCTATGTGAAGAGGTGCTTTGGCATACATCAGTTCCCTTTGCC |
| | GAGAACATGTCTCTAGAATGTGTGTATCCATCAATGGGCATCTTAACACAGGTG |
| | GAGTGGTTCAAGATCGGGACCCAGCAGGATTCCATAGCCATTTTCAGCCCTACT |
| | CATGGCATGGTCATAAGGAAGCCCTATGCTGAGAGGGTTTACTTTTTGAATTCA |
| | ACGATGGCTTCCAATAACATGACTCTTTTCTTTCGGAATGCCTCTGAAGATGAT |
| | GTTGGCTACTATTCCTGCTCTCTTTACACTTACCCACAGGGAACTTGGCAGAAG |
| | GTGATACAGGTGGTTCAGTCAGATAGTTTTGAGGCAGCTGTGCCATCAAATAGC |
| | CACATTGTTTCGGAACCTGGAAAGAATGTCACACTCACTTGTCAGCCTCAGATG |
| | ACGTGGCCTGTGCAGGCAGTGAGGTGGGAAAAGATCCAGCCCCGTCAGATCGA |
| | CCTCTTAACTTACTGCAACTTGGTCCATGGCAGAAATTTCACCTCCAAGTTCCCA |
| | AGACAAATAGTGAGCAACTGCAGCCACGGAAGGTGGAGCGTCATCGTCATCCC |
| | CGATGTCACAGTCTCAGACTCGGGGCTTTACCGCTGCTACTTGCAGGCCAGCGC |
| | AGGAGAAAACGAAACCTTCGTGATGAGATTGACTGTAGCCGAGGGTAAAACCG |
| | ATAACCAATATACCCTCTTTGTGGCTGGAGGGACAGTTTTATTGTTGTTGTTTGT |
| | TATCTCAATTACCACCATCATTGTCATTTTCCTTAACAGAAGGAGAAGGAGAGA |
| | GAGAAGAGATCTATTTACAGAGTCCTGGGATACACAGAAGGCACCCAATAACT |
| | ATAGAAGTCCCATCTCTACCAGTCAACCTACCAATCAATCCATGGATGATACAA |
| | GAGAGGATATTTATGTCAACTATCCAACCTTCTCTCGCAGACCAAAGACTAGAG |
| | TTTAAGCTTATTCTTGACATGAGTGCATTAGTAATGACTCTTATGTACTCATGCA |
| | TGGATCTTTATGCAATTTTTTTCCACTACCCAAGGTCTACCTTAGATACTAGTTG |
| | TCTGAATTGAGTTACTTTGATAGGAAAAATACTTCATTACCTAAAATCATTTTTC |
| | ATAGAACTGTTTCAGAAAACCTGACTCTAACTGGTTTATATACAAAAGAAAACT |
| | TACTGTATCATATAACAGAATGATCCAGGGGAGATTAAGCTTTGGGCAAGGGCT |
| | ATTTACCAGGGCTTAAATGTTGTGTCTAGAATTAAGTATGGGCATAAACTGGCT |
| | TCTGAATCCCTTTCCAGAGTGTTGGATCCATTTCCCTGGTCTTGGCCTCACTCTC |
| | ATGCAGGCTTTCCTCTTGTGTTGGCAAGATGGCTGCCAACTCTTGGCAATTCATA |
| | CATCCTTGTTTCTGTCTGGTAGAGAGTTTGCTTCTCAAATGGAGCAAACAAATTT |
| | GATTATTTTTTCATTGTTAAATAGGCAACATGACCAGAAAGGATGGAATGGCTT |
| | AAGTAAACTAAGGGTTCACTTCTAGAGCTGAGAAGCAGGGTCAAAGCACAATA |
| | CTGGGCAATTCAGAGCATGGTTAGAAGAGGAAAGGGGAGTCTCAAAGCTGGAG |
| | AGTTTACCAACAAATATTGACTGCAGTGATTAACCAAGACATTTTTGTTAACTA |
| | AAAAGTGAAATATGGGATGGATTCTAGAAATGGGGTATCTCTGTCCATACTTCT |
| | AGAATCCACTCTATCAGCATAGTCCAGAAGAATACCTGGCAGTAGAAGAAATG |
| | AATATTCAAGAGGAAGATAAATGCGAGAGGGCAATCCTTTACTATTCTCATATT |
| | TATTTATCTCTCATTCTGTATAGAATTCTTGCCGCCATCCCAGGTCTAGCCTTAG |
| | GAGCAAATGTAGTAGATAGTCGAATAATAAATAACTTAATGTTTTGGACATATT |
| | TTGTCTACTTTTGAGAATTATTTTTAATATGTAAATTCTCTCAAAAGGGTCAGGC |
| | ACCTAGTTATTATTTTTTAATGATTATGTGAAAGTTGAATATAATATACCACTAA |
| | AAGTGACAGTTGAAAGTGGTGGCATAGGACGGTAGGGTAGAAATTTGGGAGGG |
| | AAAAAAGAAATTGGGAGGGTACAGGCAACAGGAGAAAGGAATCAAACCACAG |
| | AAAAATACAAAGGGAAACTTCTGCTTCACTATTCAGACAAAGACAGCCCTAAT |
| | GACATCACCAACAGTCAAAGCAATTAGAGACCATACCTAATATTGTTTAAATTC |
| | TAGATGTAGGCTAACAATGAAAAGTATTTGCCAAACTGAATAAAACTGTCATG |
| | GTTACCTTGAAAGGACAATGGTTATTGTTAAATATAGTGATCATTCATGTCTAA |
| | AAGATTCATTATTTATCTCTAAAGATTTCTAAAGACCACCATCTAGAAAAGATT |
| | CATTATGAAGGCTGTATTTAAATATCAAAGTTGTGGACTTCATGATAATCTTAA |
| | ATAAAGCAAATCCAAATTCTCCTGTTGCCTAGACAGATTCTAAGATGTAATTTA |
| | CACTTTTAAGCTAATTAGTGAGTATTTTATGATTTTAGCCTTAAACACCATGTAT |
| | GCCAAATAATGCACTTGTTTTGTGAATTACAGAAATGGTAAGTGCCCACATTTC |
| | TGTGAATTATAAAATTTGTGAGTTTCTTTTAACCCTTTTCAGGAGTGAAAAAATA |
| | AAAACGACCATTTCCTGGTTGTGCTTAAGTATATGCAAGAAGGGTAAACTCTCA |
| | TTTTTATTATGTTTGCTTAAAGATCTTTTTATACCTGGATTCATGAAATGTTTCCA |
| | CAAATATATTAGTGTAACAAACTTGAAAGGCAGTTTACAAGAAAGCACTCTACT |
| | ATCAGATCAATCAAAGATTCTGTGAGTGAATTTATTGGTTTGCATGGTGAAGCA |
| | AGCTTAGCATCAATTAAAAGGTAAATAATTTCTTTTCTGAATGGTAAAGACAAT |
| | CAAAATATTACTTTCTGGAAAACTCCAATAACCAAATTCTCAATGATTAGTGTA |
| | TGTGAGCAGGAAAACATTTTTACAGTTGTAGTATGGGGAAATATAAATCCAATT |
| | TTAAGAGAGAAAATTATGACTGGGTGTGGAAGGGACAGTATAGTCAGATACCA |
| | TTGTCATGGTGGTTTTTACTGGGAACTTCATGAAAGACTTTTGTAGCAAACCACT |
| | GCAGTATTGCAAAGCCTCCAGAACATTTGGAACTTGTCTCTTTTTCCTTGTGTGT |
| | GTTTGTGTTTTTGGTCTCTCATTCAAAATATTGATGAGAACTATTTACTCTGTCC |
| | TTTCTTCTCTATATATTCTTCCTCTACAGAGTGTAGGGTTTTTTCAGGAATTTGG |
| | AGCCATCTGAAGTCCTCCCAAAAATTCTCTGACGTCTTCTGATGCTCCTGTTATA |
| | CCCTCAGGGGTAATGCTTGTGAAATTCCATTCATTCATTTTCTTTCTCTGGACAT |
| | CTTTACTTACCAAAGCACTTTCATTGTCATCTTTTTAACATCATTCTTAATTCGTG |
| | ATAGTTTTGGGACTCTCCCTAGTGTATGTTTCTCCCCCTCTACTCTTTTGCACCTA |
| | TGATTCTGATTGTTACTAAGAAAGCAGATGAAAAACAGATCCACAGAATAAAC |
| | GATCAGAATTCCAGTAAATTCTATTTTAAATACAGATACTTTTTACAAGTTGCTG |
| | CTTTGGAAGCAAAATGCTTCTTAAGTTTTACATATATATATATATATATACATAT |
| | ATATATACACATATAATTTATATCGATGGATAATACATTAAGAATCTATGCTTC |
| | CTTTGAATGCCATTAATATTTATGTTAAAGTAACCAATGAAAGGAAATTACTTT |
| | GTTATAATAAGATAGGAAGACTTGTTAATGGAGTACACAGTTTTGTCAGGGAAA |
| | GAACACATCTTATTGAACTATGATGACTATGCATTGACTATATTATTATAAGAG |
| | ATACCTTCAAACTTTATTTAAAGAACTTTAGGTATAATATGTTGAGAAAATAAA |
| | ATAGAAATTTCATTTACTTGTAATCATGCTTAAAATGGGAGGCAGGTAGGTGAA |
| | GATATAATTTTTAGTAAAAACTCCAATTTATGTTTTAAGTAATTCAGTGTATTAC |
| | TAAAATACTATATATATAAACTTAAAATACATGGGTTATCAATTTAAAAGACAA |
| | AGTAAGTAAAAATACTTTTAGTAGGCATTCGTGGATTGTGAACATCCAAGTTAT |
| | ATTGGTTTGTATAGAATGGCATTAAGTAAAAATTACAGCTGTATAACAGTAGTT |
| | TTCTAAATTGAGAGAGTCCACATTGTAATTAGAGATCACTGTGACCAAAATGCT |
| | TCTCCTTGATTTATAATGATGTACTGTATTTTGTACTGCTTATATGAAATTTCAG |
| | CAAGATTGACGATATTATAAAGATGCTTATAAAGTGTAAGTGGAGACGCTAAA |
| | TTGTGAGTACAAAGTTTCTTTTTCACAACAGTGATAAGAAAATATCTTTAAAAA |
| | ATATAAGACAATATAAACATGTCATCATTAGTTTAGCTACTATTAAAATGTAAC |
| | ATCTAGAAAGTACTGATCTCCACCTTCAGACTTCTGTATAAGTATATTTTTTCAC |
| | TGATCTGTTCATTAGAGTTCTTCCAGCCAAGACTCTGGGCTCTTAAAACATGTAT |
| | CTGAAAACTAAAAACAAGTTAATTTTTTTAAAAGCTTCTCTATTTCTAGTGATTC |
| | AATAGGTAGAAAAATAGCTTCTAGAATTAACTGCAATGCTTTCTAAGGAAATTT |
| | TATAAATCCTCAAGGTCGGTTTACACATATTTTTCCAGATTCAGAGCACTAACT |
| | ATCTTGTAAGATGTAAGAAAAGGTCCATTTGGAAGTATGAGTAATAAATGTCTG |
| | GGATAATTCTGGTTTATTTCGTATTATCCTTGTTAGAATAAGTTATATGGTCAAC |
| | CTGTTCAGAACACTTTTTCTAGTGTTAGTGTGTACTTTTGGATTTTTGGTTCTTGT |
| | AGGGTATAGAAATATTTTCCTTTGTCTTGTATTCTGTTGTTTTGAATGAATAAAA |
| | CACAATGTTTCACGATCACTACTTTCATTTGCCATGGAGAAATAGCAGGGAAAA |
| | ATTTCTACAGAATAAAATTAACTGATGAATTACATGCAGAAAAAATTCAAATCA |
| | ATGATACATTGTAATTTTTATCTCAATGCAATGTTCTTTGTATTTTATTTTATTAT |
| | TATTTTTTTGAGACGGAGTTTCACTTTTGTTGCCCGGGCTGGAGTGCAATGGCAC |
| | AATCTCGGCTCACCACAACCTCTGCCTCCCGGATTCAAGTGATTCTCCTGCCTCA |
| | GCCTCCTGAATAGCTGGGATTACAGGCATATGCCAACATGCCTGGCTAATTTTG |
| | TATTTTTAGTGGAGACGGGGTTTCTCCACGTTGGTCAGACTTGTCTTGAACTCTG |
| | GACCTCAGGTGATCCACCTGCCTCAGCCTCCTAAATTGCTGGGATTACAGGCAT |
| | GAGCGACCACTCCTGGCCTTGTTCTTTGTATTTTATAAGTGCATGTAGTGCAAAG |
| | GGTCAAAGGGCTTTACAGGTTTTTTGTTTGTTTGTTTTTGTTTTTCCCGAAACAT |
| | AGTAGTCCCTTGCCCTTCCTCATTTTTGTTACCTTGAGACAACAAATTTTACTAC |
| | TTCTAACTCATTATTTTATTTATGTTCACTTTTCTGAATAGCATGCTTATGACACT |
| | AATACTTTTTTTTTCAATTTTAGACATTCATTATTCATTTAGATGTCTTTCTCTCC |
| | CCAAACTCACCACATAAAATACTCTTCTCATGTCTCTTTCAGAAATATTTGTATT |
| | AAAATATGATTATATCAATATTTGGCATTTATTTCTTATGACCTTGCCAGTACTC |
| | TTAGTTAAACTACATGGTAAAAATGATTTTGCTTTCCCTCCTACATAACTTTTTT |
| | TCCACCTAGAGCTAATAATTGTCATTCTGGGGACTGACTTTTTCTGTATTTACCA |
| | TAAATTGACCTGAAACTCCCCTGTGATGCAGCAGGAATTCTACCAACGTCAACT |
| | TCCTTAGAAAGACTCCATTAGAAGCTTGACTTGGGGCTAGAAGGAGAGGCACA |
| | CAACTGCCATCCTGGTGTCTCCCTTCATCCAGAAAAAGGGGGAGGAATACATGA |
| | AACCTAGAATCCACTCTAAAACATTTTCCAGAACAAAAGGACATGTGTTTCCGT |
| | GTTGTAAATGTTTAACGAGTGCCCATAACAAGGAATAATAAGTCTATTATGTTT |
| | GCTTTTGTGTCTGTAAAAGTTGGGGGTATTGGTTGTAAGCACGAAAACAGATAC |
| | TGACTGTTGAAGAAAAAAAAAAATACGAGGTCAGGAGTTTGAGACCAACTTGG |
| | CCAATATGGTGAAACCCTGTCTTAGTAAAAATAGAAAAATTAGCCAGGCCTGGT |
| | GGCACGCACCTGTAGTCCCAGCTACTTGGGAGGCTGAGGCAGAAGAATCGCTT |
| | GAACCCGGGAGGCAGAGGTTGCAGTGAGCCAAGATCGCACCACTGCACTCCAC |
| | CCTGGGCAACAGAGCGAGACTCCGTCTCAAAAAAAAAAAAAAAAAAAAAAAA |
| | AAAAAGTTAAGTATTTGAACATAGGGGTGGCTCATAGAATTCCCAGGACACCC |
| | GATGGAGTAGGCTTGCAAAACACAACATGTGGCAACTCCAGTGGGAAACGAGG |
| | CAGGAAACACTCGTTTCCTGCAGAAAGCAACAATTTGGGCTTCGATACCCTCCC |
| | TAGAACACAGGGCAGTGAATCTGAGCAGCATCAGTACCCCACGTTCGGATGAG |
| | TCCTGAGCCCCTATTTTTATTCACTGACTTATTCCAAAATCAGTGTCTCTTAAAT |
| | ATATCTGGAAGGCAGCAGCTTGTATCTCCCCCTTCAGCTTCCATAGTGGCAGTC |
| | AGGGTACAACTTACTTTCCAAACAGAACACACTGCGACATTCCCTCCAGGCTCG |
| | TTGAAGAACTTCAACTGACAAATGTCCCTCCTCGACCAGATGATAGTTTTCTTA |
| | AAGGCAGGGTTTAATATACCCTTTTATAAATGTTTCAAGGCCCTGTGTAATACC |
| | TGAGTTTATTCCAGATGTAACTAAATATATCCAAGATTGTTTTAAAATAAATTG |
| | CTGAAAAAACAAATAAATACAGTTAGTATCTATATCAATATTCTCAGTTGGCAG |
| | TTTTGCAATAATGGCCGATAGTTCATTTTTAGTAACACTATTGACATTGCATTTG |
| | GATATTAGGGTTTACTAATCATCCGCATGTATACATTGCATATTTTTCTAGACTT |
| | TAACTTTATTCAAATCTATTGATTTTTAAACCTGCAACTTATGTCTAGACACAGG |
| | TATACCTTTACAAGAACTACCATTTTTTTTGGTAACATACTACCTCCAAAATTTC |
| | AAGTAAGAAGTTGATTTTTGTCCATTTTTAAATGGAAAACTTGTAATCAAAATG |
| | CCACAAAATTATACTGTGTATCATTTGACCTATAGAAACCAATATTATTACAGG |
| | AAGAAAGCAGAGCCAATCTTCTACCTGTGGTCAAATAAGTGGAGGCCCTTTCTA |
| | GACTAAGTTCTCATGAGTTTAAAATACCAAGCATAAGTTCTCCAAATTCCTGAA |
| | AAGGAAGCCTTGTGTTGTATTGCCCAGCCATATTTGTAAGACATAAAAATAAAA |
| | CTTGAGAAGAAGCTATGATAACTTACTTTCTTCATTCTTCAAAATTTACATAATC |
| | TCAACTGATTTTATGTTTTTATGAAAATGCATTCTTAAGATATATCCTTATTCAA |
| | TCATGTATTCATTACATCCTTTATGCCAGGTATCCAAAAGTACTTACAGTGACTA |
| | AGACCATTATTCTTTGATCAGCTGCCTGAGTAAGACTTTGAGCTCTCCAATATAC |
| | TCTCAGTGATACTAAGTTTTCTGAGTAACAGCTTTGGATGTGGCTTCAGTTGAGC |
| | TGATTTATCCCACACTTTATTTTTATCGTATAATGGTCCTCAGAAGCAAATTTTG |
| | ATTTTAGCTCACATAAAAAATGTACAAAGAAATGTAATGGCTCAGTAGCTTCTA |
| | GAGATAGAGATTACTCTTCTAACCTTTCTGTAATTTTGTATGTCTATTTTATAAT |
| | TCTTTCAATGTCTAATGAATAGCTATCTTTTTTTGAGACGGAGTCTCGCTCTGTC |
| | GCCCAGGCTGGAGTGCAGTGGTGCGACCTCGGCTCACCGCAAGCTGCGTCTTCC |
| | AGGTTCACGCCATTCTCCTGCCTCAGCCTCCCGAGTAGCTGGGACTTCAGGCGC |
| | CCACCACCATGCCCAGCTAATTTTTTTGTATTTTTAGTAGAGACGGGGTTTCACC |
| | GTGTTAGCCAGGGTGGTCTCGATCTCCTGACCTCGTGATCCGCCCGCCTCGGCC |
| | TCCCAACGTGCTGGGATTACAGGAGTGAGCCACCGCGCCCGGCCTCCTTAGTTT |
| | CTTAAGGTGGAAGCCTAGATTATTGATTTTATATGTTGTTTTCTTTTCCAATAGT |
| | GGCACTTAATGCTATAAATTTCACTTTGTTCCACAAGTTTTGGTAAGCTCTATTT |
| | TTATTTTCATTTAGTCCAAAATATTTTAAAATTTCTTTTGATATTTCTTCTTTGAG |
| | CCATGAATTATTTACAATGTGTTGTTTAATCTCTATATATTTTGGGATTTTTCTAC |
| | TTTATATCTCTTACAGATTTCTAACTTAATTTCATCATGTTTTAAAAACATTCTTT |
| | GTATAATTTCTATTCTTTTAAATTTTTCAGGTGTATTTTATGGCCCAGAATATGG |
| | TCTATCTTGTAGAATGTTTCATGTGATCTTAAGAAGAATGTTCATTCTGCTGTTG |
| | AGTGTAATATTCTACAAATGTCCATTAGATTAAACTGATTGATACCACCGTTCA |
| | GATTATCTATATCCTTTCTGATTTTCCCTCTTCTTGATCTATCACATACTGACAG |
| | ATCAAGTGATCAAGTCTCGTTAAAGACTGCAAGTAAAATAGTGGATTTTTCTAT |
| | TTCTCCTTGCAGTTTTGTTAGTTTTTGTCTCATGTATCTTGATACTCTTGTTAGTA |
| | CATATACTTTCAGAATCGTTAGGTTTTCTTGGAGAATTGACCCCTTTACCACATG |
| | TAATGTCCCTTTTATTCTTGATAATCTTTCTTGTTCTGTCTGCTTTTTCTGATATT |
| | AACATAACTTTCAGTTTTTTAAAAAATTAACATTAGCATCTCACATCTTTATCCT |
| | TTTAATTTTAAATTATCTAAATATTTATATTTAATGTGCCTTTCTTATAGACAAT |
| | GTATAGTTGCGTCTATTTGTAATTTCCCCACTTTTCTTACTTAAAAATGTTGTAG |
| | ATATATAGGAGTTGTATATATTTGGGGGGTACATGTGATGTTTTGATACCTGTAT |
| | ACAATATGTAATGATCATATTGGGTAATCGTGATATCTGTCACCTCTAACATTC |
| | ATCTTTTTTGTGTGTTTAAACCCACCACTTCTAATTGGTACATTTAGATTATTCA |
| | AATTTAAGTGATTATTGATATAGTTGGATTAATATCTACTATGTTTGTAACTTTT |
| | CTATCCTTGCACTCGTTCTTTCTTTTTTATCCTCCTTTTTCTGTGTTCTCTGATTTT |
| | AACTGGGGTTTTTACATGATTTAATTTTCTCTCGTGGCATATCTTTCATTGATCA |
| | ACCTAGGTTTTTCTCCTTTTCCCCTCTTTTTTTTGGTATTTATTCTATTTAGTGTTA |
| | TCTGAGCTACCTGAGTTGGTGTCTATCACTAATTTTGGCAAGTTCCCAGACGTTA |
| | TTACTTCTAACATTCTTTTGCTCCATTCTTTCTTCTTCTTCAATTATTCCATAGTC |
| | TTGAATATTCTGGGTTTTTCCCACTCTTTGAATTTTAGTTTGAAAAGTTTCTATTG |
| | GCCTAGCTTCAAAGTCATTCATTCTTCCTTCGGGGTTCCAAGTCAACTGATAATT |
| | GCATCAAAGATATCCTTCCTTTCTATTACTATGTTTTTTATTGCTACCATTTCTTT |
| | TTTATTCCTTCTTAGTGTTTCCATCTTTCTTCTTACATTATCCATCTGTTGTCTATT |
| | TTTTTCATGAGAGCTCTTAACATATTAATGATAAGTTCCATGTCTGATAATTCTG |
| | ACACGTGTCATGTCTCTATCTGGTTCCAATGATTGCTTTATCTCTTCAGACCATG |
| | ACTTTTCTTGCCTTTTGACGTTCTTTGACATTTTTTTTGAATTTTTTGTTGCAAGC |
| | CAGATCTGGTGTGTTATGTAATAGGAACAGGTAAATAAGTCTTTAGCTTGCAGA |
| | CTTATCTTAATCTGACTAACTATTAGACTGTGTTTAAAGTCTGTTATAACCATAG |
| | GTGCTAAATTTCTTCAAATTCCTCTAGTGTCTTTGTTTTGTTTGTTCATGTGTTTT |
| | TCCCCTTCTTGAGTTCAGGCTTCCCTAAGTGCTCCTCTTCAGAGAGACTTTCTGT |
| | CTTTCAGCTCTTTCCTCTGCAATTCACTGTTACTATACTGGAGCCCTGTTGGTGT |
| | AGTACTAAGCTGTGGGAAAGGAGAGTGCTCTGTAATCTTACAGTGAAATCTCAG |
| | TCTTTTAGTGGGTCTGTGTCTGGGACATTCACAGAGCTTCTCCAGTGGTATTGCT |
| | TCCTCATCCTCAACTCTCTTTCCTGGCTGCAGCATTCCCAATGTATTTCTTTGAA |
| | GGCCTGCCCCCTGTTGACTGTTATTTTCCCTCTTTCCTTAAGTGGGACAGGGAGA |
| | CTTCAGGGGCTGGGATGAGGTTTGGGAATTGTGCTTGGCAGAGTCCTTTCCATC |
| | TTTGTTACCAAGAAGGTTCATGGCTTATTTCTCAATGGATGTCCCTCTCTATCTG |
| | TTGCCAGAGCCACGAGGAAATTTTTCTTGGATCCTCATAATGAGAACCTTGGAG |
| | TTTCCTACTGGAAAAGCCCTTGAATGTGTGGAGTGCCTCAAGAGCACAGCCCCC |
| | ATGGGTTTCTTGCTCACACCAGTCCACAAACAGATGCCAGCAATTCACCCAACT |
| | TACCATATAAAGGCTCATACTAGTTTATGGCTCCAGTGCTTTGACTCCAGATAA |
| | ATGGCTATTGGTTGCGTATCTCTCTGGATGTATCTGTATCTCCAGATTTTGGGGT |
| | GGCAGTTTGCTCAGGACCTTGGTTCTCTAATAGGTCTAATAAGAAAAGTCATTG |
| | ATTTTCAGCTTTCCAACTTTCCAGCTTTGTCTTGTTATAAGCATGGCAGCAACAT |
| | CTTCCATGCCTTAACATGATGACACTAAAGGCAGAAGTCGATCTCCATGTATAA |
| | ACATTTTAACACATATGTTTTTTGTTATCGTGGTTTCTGACCTGTCTCTTTGCCCT |
| | GACTTTCTGATACTGCACTAGGGTTCCTGTTGCTGGACTCCATTCCATATGACTT |
| | GCTCTCGTCTAGGCTGCTCTTTGGCTCATCTTTATAAATCATGATCCAAAATGAA |
| | GCACATATTTATTTTTTAAATAAATATGAAATGAAGTATAGACATCAAACTGAA |
| | GATGAGTAGATCATACTGAGTTTCACTGTCTGTGCTTGGATCAACATCAGGCCT |
| | TATACAAATATTCAAGTCCAGAGGCAAAAGGTAATAAGGAAAATTTGTAGCAC |
| | AAGCCACAAGGAGATAACATGTCAAGTCTATGCGATTGGAAATAAACTAAAGA |
| | TGAACTGCTGGGGATGCTCACTCATCACAGAGCTCAGTCTAAAGCACCAGATTT |
| | CACAAGCATTTTTTGGGGGAAATTCTGTTAAAATGAAATATGAGTCACATGGTG |
| | GTGTTTCACTCATCATATGTGTTCAATATTAATTCATTTTAAGGTTTAGTTGCAC |
| | AAAAGGTAAATGAGAATTAGAAGACTCCATGGGTAAGAGGAGCCACAGAAGT |
| | AAAGCATTGTCAAGGGTTCTATGTCTATATATTTAGATATTAGGCTTCTGAGAA |
| | AAAAACACAATAGGAAGGAAGATGAACACAACAGAGGGCAGAAGGTCTATAC |
| | GTCCTGAGGCCTTTTATGCAACGTTTGTTTGTGGAATGTTTTTTAAGAATGTGTG |
| | AGAGTCATTTTAATGTGAAATAAAGACCTACGTCTACA (SEQ ID NO: 91) |
| | >NP_006557.2 CD226 antigen isoform a precursor [Homo |
| | sapiens], amino acid sequence |
| | MDYPTLLLALLHVYRALCEEVLWHTSVPFAENMSLECVYPSMGILTQVEWFKIGT |
| | QQDSIAIFSPTHGMVIRKPYAERVYFLNSTMASNNMTLFFRNASEDDVGYYSCSLY |
| | TYPQGTWQKVIQVVQSDSFEAAVPSNSHIVSEPGKNVTLTCQPQMTWPVQAVRWE |
| | KIQPRQIDLLTYCNLVHGRNFTSKFPRQIVSNCSHGRWSVIVIPDVTVSDSGLYRCY |
| | LQASAGENETFVMRLTVAEGKTDNQYTLFVAGGTVLLLLFVISITTIIVIFLNRRRRR |
| | ERRDLFTESWDTQKAPNNYRSPISTSQPTNQSMDDTREDIYVNYPTFSRRPKTRV |
| | (SEQ ID NO: 92) |
| Human DR3 | >NM_003790.3 Homo sapiens TNF receptor superfamily |
| | member 25 (TNFRSF25), transcript variant 2, mRNA, |
| | nucleic acid sequence |
| | GAAGGCGGAACCACGACGGGCAGAGAGCACGGAGCCGGGAAGCCCCTGGGCG |
| | CCCGTCGGAGGGCTATGGAGCAGCGGCCGCGGGGCTGCGCGGCGGTGGCGGCG |
| | GCGCTCCTCCTGGTGCTGCTGGGGGCCCGGGCCCAGGGCGGCACTCGTAGCCCC |
| | AGGTGTGACTGTGCCGGTGACTTCCACAAGAAGATTGGTCTGTTTTGTTGCAGA |
| | GGCTGCCCAGCGGGGCACTACCTGAAGGCCCCTTGCACGGAGCCCTGCGGCAA |
| | CTCCACCTGCCTTGTGTGTCCCCAAGACACCTTCTTGGCCTGGGAGAACCACCA |
| | TAATTCTGAATGTGCCCGCTGCCAGGCCTGTGATGAGCAGGCCTCCCAGGTGGC |
| | GCTGGAGAACTGTTCAGCAGTGGCCGACACCCGCTGTGGCTGTAAGCCAGGCT |
| | GGTTTGTGGAGTGCCAGGTCAGCCAATGTGTCAGCAGTTCACCCTTCTACTGCC |
| | AACCATGCCTAGACTGCGGGGCCCTGCACCGCCACACACGGCTACTCTGTTCCC |
| | GCAGAGATACTGACTGTGGGACCTGCCTGCCTGGCTTCTATGAACATGGCGATG |
| | GCTGCGTGTCCTGCCCCACGAGCACCCTGGGGAGCTGTCCAGAGCGCTGTGCCG |
| | CTGTCTGTGGCTGGAGGCAGATGTTCTGGGTCCAGGTGCTCCTGGCTGGCCTTG |
| | TGGTCCCCCTCCTGCTTGGGGCCACCCTGACCTACACATACCGCCACTGCTGGC |
| | CTCACAAGCCCCTGGTTACTGCAGATGAAGCTGGGATGGAGGCTCTGACCCCAC |
| | CACCGGCCACCCATCTGTCACCCTTGGACAGCGCCCACACCCTTCTAGCACCTC |
| | CTGACAGCAGTGAGAAGATCTGCACCGTCCAGTTGGTGGGTAACAGCTGGACC |
| | CCTGGCTACCCCGAGACCCAGGAGGCGCTCTGCCCGCAGGTGACATGGTCCTGG |
| | GACCAGTTGCCCAGCAGAGCTCTTGGCCCCGCTGCTGCGCCCACACTCTCGCCA |
| | GAGTCCCCAGCCGGCTCGCCAGCCATGATGCTGCAGCCGGGCCCGCAGCTCTAC |
| | GACGTGATGGACGCGGTCCCAGCGCGGCGCTGGAAGGAGTTCGTGCGCACGCT |
| | GGGGCTGCGCGAGGCAGAGATCGAAGCCGTGGAGGTGGAGATCGGCCGCTTCC |
| | GAGACCAGCAGTACGAGATGCTCAAGCGCTGGCGCCAGCAGCAGCCCGCGGGC |
| | CTCGGAGCCGTTTACGCGGCCCTGGAGCGCATGGGGCTGGACGGCTGCGTGGA |
| | AGACTTGCGCAGCCGCCTGCAGCGCGGCCCGTGACACGGCGCCCACTTGCCACC |
| | TAGGCGCTCTGGTGGCCCTTGCAGAAGCCCTAAGTACGGTTACTTATGCGTGTA |
| | GACATTTTATGTCACTTATTAAGCCGCTGGCACGGCCCTGCGTAGCAGCACCAG |
| | CCGGCCCCACCCCTGCTCGCCCCTATCGCTCCAGCCAAGGCGAAGAAGCACGA |
| | ACGAATGTCGAGAGGGGGTGAAGACATTTCTCAACTTCTCGGCCGGAGTTTGGC |
| | TGAGATCGCGGTATTAAATCTGTGAAAGAAAACAAAACAAAACAAAAACGGCT |
| | TCTTGGCGTTTCTGCGGGGCTGGGGTGTTAAGTGGACTGGACTTTTCTCGAGGG |
| | ATTCGAAGGGGACGGGAATCTTGTCACCCCGGGATCTGGCACCCATGGTGGAG |
| | TCCAGTGTGGCCTTAGCTCCCAAGCCTGCCCCTCCCGAGTCCACTCTGGCTCAA |
| | TTACCCCGAGAAGGAGAGAGCAAGTCGCGGCCACAGCGAGTGAGTGAACCGGA |
| | GCCCAGATGAGAGCGCTTTAATGGGGCTGCGAGGTGGCGGAGACAGGGTCGGG |
| | ATGGGGTGCAGCAGTTGGAGACACAGGGTCAGGGCCCCTCATCCTCTATTCACT |
| | CCACCGGGGCAGTGAAAGGGTCCCGGCAGCGAGTGGGTC |
| | (SEQ ID NO: 93) |
| | >NP_003781.1 tumor necrosis factor receptor superfamily |
| | member 25 isoform 2 precursor [Homo sapiens], amino acid |
| | sequence |
| | MEQRPRGCAAVAAALLLVLLGARAQGGTRSPRCDCAGDFHKKIGLFCCRGCPAGH |
| | YLKAPCTEPCGNSTCLVCPQDTFLAWENHHNSECARCQACDEQASQVALENCSAV |
| | ADTRCGCKPGWFVECQVSQCVSSSPFYCQPCLDCGALHRHTRLLCSRRDTDCGTC |
| | LPGFYEHGDGCVSCPTSTLGSCPERCAAVCGWRQMFWVQVLLAGLVVPLLLGATL |
| | TYTYRHCWPHKPLVTADEAGMEALTPPPATHLSPLDSAHTLLAPPDSSEKICTVQL |
| | VGNSWTPGYPETQEALCPQVTWSWDQLPSRALGPAAAPTLSPESPAGSPAMMLQP |
| | GPQLYDVMDAVPARRWKEFVRTLGLREAEIEAVEVEIGRFRDQQYEMLKRWRQQ |
| | QPAGLGAVYAALERMGLDGCVEDLRSRLQRGP (SEQ ID NO: 94) |
| Human DcR3 | >NM_003823.4 Homo sapiens TNF receptor superfamily member |
| | 6b (TNFRSF6B), mRNA, nucleic acid sequence |
| | GGACTTGGGCGGCCCCTCCGCAGGCGGACCGGGGGCAAAGGAGGTGGCATGTC |
| | GGTCAGGCACAGCAGGGTCCTGTGTCCGCGCTGAGCCGCGCTCTCCCTGCTCCA |
| | GCAAGGACCATGAGGGCGCTGGAGGGGCCAGGCCTGTCGCTGCTGTGCCTGGT |
| | GTTGGCGCTGCCTGCCCTGCTGCCGGTGCCGGCTGTACGCGGAGTGGCAGAAAC |
| | ACCCACCTACCCCTGGCGGGACGCAGAGACAGGGGAGCGGCTGGTGTGCGCCC |
| | AGTGCCCCCCAGGCACCTTTGTGCAGCGGCCGTGCCGCCGAGACAGCCCCACG |
| | ACGTGTGGCCCGTGTCCACCGCGCCACTACACGCAGTTCTGGAACTACCTAGAG |
| | CGCTGCCGCTACTGCAACGTCCTCTGCGGGGAGCGTGAGGAGGAGGCACGGGC |
| | TTGCCACGCCACCCACAACCGTGCCTGCCGCTGCCGCACCGGCTTCTTCGCGCA |
| | CGCTGGTTTCTGCTTGGAGCACGCATCGTGTCCACCTGGTGCCGGCGTGATTGC |
| | CCCGGGCACCCCCAGCCAGAACACGCAGTGCCAGCCGTGCCCCCCAGGCACCT |
| | TCTCAGCCAGCAGCTCCAGCTCAGAGCAGTGCCAGCCCCACCGCAACTGCACG |
| | GCCCTGGGCCTGGCCCTCAATGTGCCAGGCTCTTCCTCCCATGACACCCTGTGC |
| | ACCAGCTGCACTGGCTTCCCCCTCAGCACCAGGGTACCAGGAGCTGAGGAGTGT |
| | GAGCGTGCCGTCATCGACTTTGTGGCTTTCCAGGACATCTCCATCAAGAGGCTG |
| | CAGCGGCTGCTGCAGGCCCTCGAGGCCCCGGAGGGCTGGGGTCCGACACCAAG |
| | GGCGGGCCGCGCGGCCTTGCAGCTGAAGCTGCGTCGGCGGCTCACGGAGCTCC |
| | TGGGGGCGCAGGACGGGGCGCTGCTGGTGCGGCTGCTGCAGGCGCTGCGCGTG |
| | GCCAGGATGCCCGGGCTGGAGCGGAGCGTCCGTGAGCGCTTCCTCCCTGTGCAC |
| | TGATCCTGGCCCCCTCTTATTTATTCTACATCCTTGGCACCCCACTTGCACTGAA |
| | AGAGGCTTTTTTTTAAATAGAAGAAATGAGGTTTCTTAAAGCTTATTTTTATAAA |
| | GCTTTTTCATAAAA (SEQ ID NO: 95) |
| | >NP_003814.1 tumor necrosis factor receptor superfamily |
| | member 6B precursor [Homo sapiens], amino acid sequence |
| | MRALEGPGLSLLCLVLALPALLPVPAVRGVAETPTYPWRDAETGERLVCAQCPPGT |
| | FVQRPCRRDSPTTCGPCPPRHYTQFWNYLERCRYCNVLCGEREEEARACHATHNR |
| | ACRCRTGFFAHAGFCLEHASCPPGAGVIAPGTPSQNTQCQPCPPGTFSASSSSSEQC |
| | QPHRNCTALGLALNVPGSSSHDTLCTSCTGFPLSTRVPGAEECERAVIDFVAFQDISI |
| | KRLQRLLQALEAPEGWGPTPRAGRAALQLKLRRRLTELLGAQDGALLVRLLQALR |
| | VARMPGLERSVRERFLPVH (SEQ ID NO: 96) |
| Human | >NM_012206.3 Homo sapiens hepatitis A virus cellular |
| TIM-1 | receptor 1 (HAVCR1), transcript variant 1, mRNA, nucleic |
| (CD365) | acid sequence |
| | GACCAGGAGTCAGTTTGGCGGTTATGTGTGGGGAAGAAGCTGGGAAGTCAGGG |
| | GCTGTTTCTGTGGACAGCTTTCCCTGTCCTTTGGAAGGCACAGAGCTCTCAGCTG |
| | CAGGGAACTAACAGAGCTCTGAAGCCGTTATATGTGGTCTTCTCTCATTTCCAG |
| | CAGAGCAGGCTCATATGAATCAACCAACTGGGTGAAAAGATAAGTTGCAATCT |
| | GAGATTTAAGACTTGATCAGATACCATCTGGTGGAGGGTACCAACCAGCCTGTC |
| | TGCTCATTTTCCTTCAGGCTGATCCCATAATGCATCCTCAAGTGGTCATCTTAAG |
| | CCTCATCCTACATCTGGCAGATTCTGTAGCTGGTTCTGTAAAGGTTGGTGGAGA |
| | GGCAGGTCCATCTGTCACACTACCCTGCCACTACAGTGGAGCTGTCACATCCAT |
| | GTGCTGGAATAGAGGCTCATGTTCTCTATTCACATGCCAAAATGGCATTGTCTG |
| | GACCAATGGAACCCACGTCACCTATCGGAAGGACACACGCTATAAGCTATTGG |
| | GGGACCTTTCAAGAAGGGATGTCTCTTTGACCATAGAAAATACAGCTGTGTCTG |
| | ACAGTGGCGTATATTGTTGCCGTGTTGAGCACCGTGGGTGGTTCAATGACATGA |
| | AAATCACCGTATCATTGGAGATTGTGCCACCCAAGGTCACGACTACTCCAATTG |
| | TCACAACTGTTCCAACCGTCACGACTGTTCGAACGAGCACCACTGTTCCAACGA |
| | CAACGACTGTTCCAATGACGACTGTTCCAACGACAACTGTTCCAACAACAATGA |
| | GCATTCCAACGACAACGACTGTTCTGACGACAATGACTGTTTCAACGACAACGA |
| | GCGTTCCAACGACAACGAGCATTCCAACAACAACAAGTGTTCCAGTGACAACA |
| | ACTGTCTCTACCTTTGTTCCTCCAATGCCTTTGCCCAGGCAGAACCATGAACCA |
| | GTAGCCACTTCACCATCTTCACCTCAGCCAGCAGAAACCCACCCTACGACACTG |
| | CAGGGAGCAATAAGGAGAGAACCCACCAGCTCACCATTGTACTCTTACACAAC |
| | AGATGGGAATGACACCGTGACAGAGTCTTCAGATGGCCTTTGGAATAACAATC |
| | AAACTCAACTGTTCCTAGAACATAGTCTACTGACGGCCAATACCACTAAAGGAA |
| | TCTATGCTGGAGTCTGTATTTCTGTCTTGGTGCTTCTTGCTCTTTTGGGTGTCATC |
| | ATTGCCAAAAAGTATTTCTTCAAAAAGGAGGTTCAACAACTAAGTGTTTCATTT |
| | AGCAGCCTTCAAATTAAAGCTTTGCAAAATGCAGTTGAAAAGGAAGTCCAAGC |
| | AGAAGACAATATCTACATTGAGAATAGTCTTTATGCCACGGACTAAGACCCAGT |
| | GGTGCTCTTTGAGAGTTTACGCCCATGAGTGCAGAAGACTGAACAGACATCAGC |
| | ACATCAGACGTCTTTTAGACCCCAAGACAATTTTTCTGTTTCAGTTTCATCTGGC |
| | ATTCCAACATGTCAGTGATACTGGGTAGAGTAACTCTCTCACTCCAAACTGTGT |
| | ATAGTCAACCTCATCATTAATGTAGTCCTAATTTTTTATGCTAAAACTGGCTCAA |
| | TCCTTCTGATCATTGCAGTTTTCTCTCAAATATGAACACTTTATAATTGTATGTT |
| | CTTTTTAGACCCCATAAATCCTGTATACATCAAAGAGAA (SEQ ID NO: 97) |
| | >NP_036338.2 hepatitis A virus cellular receptor 1 |
| | isoform a precursor [Homo sapiens], amino acid sequence |
| | MHPQVVILSLILHLADSVAGSVKVGGEAGPSVTLPCHYSGAVTSMCWNRGSCSLFT |
| | CQNGIVWTNGTHVTYRKDTRYKLLGDLSRRDVSLTIENTAVSDSGVYCCRVEHRG |
| | WFNDMKITVSLEIVPPKVTTTPIVTTVPTVTTVRTSTTVPTTTTVPMTTVPTTTVPTT |
| | MSIPTTTTVLTTMTVSTTTSVPTTTSIPTTTSVPVTTTVSTFVPPMPLPRQNHEPVATS |
| | PSSPQPAETHPTTLQGAIRREPTSSPLYSYTTDGNDTVTESSDGLWNNNQTQLFLEH |
| | SLLTANTTKGIYAGVCISVLVLLALLGVIIAKKYFFKKEVQQLSVSFSSLQIKALQNA |
| | VEKEVQAEDNIYIENSLYATD (SEQ ID NO: 98) |
| Human PD-1 | >NM_005018.3 Homo sapiens programmed cell death 1 (PDCD1), |
| | mRNA, nucleic acid sequence |
| | GCTCACCTCCGCCTGAGCAGTGGAGAAGGCGGCACTCTGGTGGGGCTGCTCCA |
| | GGCATGCAGATCCCACAGGCGCCCTGGCCAGTCGTCTGGGCGGTGCTACAACTG |
| | GGCTGGCGGCCAGGATGGTTCTTAGACTCCCCAGACAGGCCCTGGAACCCCCCC |
| | ACCTTCTCCCCAGCCCTGCTCGTGGTGACCGAAGGGGACAACGCCACCTTCACC |
| | TGCAGCTTCTCCAACACATCGGAGAGCTTCGTGCTAAACTGGTACCGCATGAGC |
| | CCCAGCAACCAGACGGACAAGCTGGCCGCCTTCCCCGAGGACCGCAGCCAGCC |
| | CGGCCAGGACTGCCGCTTCCGTGTCACACAACTGCCCAACGGGCGTGACTTCCA |
| | CATGAGCGTGGTCAGGGCCCGGCGCAATGACAGCGGCACCTACCTCTGTGGGG |
| | CCATCTCCCTGGCCCCCAAGGCGCAGATCAAAGAGAGCCTGCGGGCAGAGCTC |
| | AGGGTGACAGAGAGAAGGGCAGAAGTGCCCACAGCCCACCCCAGCCCCTCACC |
| | CAGGCCAGCCGGCCAGTTCCAAACCCTGGTGGTTGGTGTCGTGGGCGGCCTGCT |
| | GGGCAGCCTGGTGCTGCTAGTCTGGGTCCTGGCCGTCATCTGCTCCCGGGCCGC |
| | ACGAGGGACAATAGGAGCCAGGCGCACCGGCCAGCCCCTGAAGGAGGACCCCT |
| | CAGCCGTGCCTGTGTTCTCTGTGGACTATGGGGAGCTGGATTTCCAGTGGCGAG |
| | AGAAGACCCCGGAGCCCCCCGTGCCCTGTGTCCCTGAGCAGACGGAGTATGCC |
| | ACCATTGTCTTTCCTAGCGGAATGGGCACCTCATCCCCCGCCCGCAGGGGCTCA |
| | GCTGACGGCCCTCGGAGTGCCCAGCCACTGAGGCCTGAGGATGGACACTGCTCT |
| | TGGCCCCTCTGACCGGCTTCCTTGGCCACCAGTGTTCTGCAGACCCTCCACCAT |
| | GAGCCCGGGTCAGCGCATTTCCTCAGGAGAAGCAGGCAGGGTGCAGGCCATTG |
| | CAGGCCGTCCAGGGGCTGAGCTGCCTGGGGGCGACCGGGGCTCCAGCCTGCAC |
| | CTGCACCAGGCACAGCCCCACCACAGGACTCATGTCTCAATGCCCACAGTGAGC |
| | CCAGGCAGCAGGTGTCACCGTCCCCTACAGGGAGGGCCAGATGCAGTCACTGC |
| | TTCAGGTCCTGCCAGCACAGAGCTGCCTGCGTCCAGCTCCCTGAATCTCTGCTG |
| | CTGCTGCTGCTGCTGCTGCTGCTGCCTGCGGCCCGGGGCTGAAGGCGCCGTGGC |
| | CCTGCCTGACGCCCCGGAGCCTCCTGCCTGAACTTGGGGGCTGGTTGGAGATGG |
| | CCTTGGAGCAGCCAAGGTGCCCCTGGCAGTGGCATCCCGAAACGCCCTGGACG |
| | CAGGGCCCAAGACTGGGCACAGGAGTGGGAGGTACATGGGGCTGGGGACTCCC |
| | CAGGAGTTATCTGCTCCCTGCAGGCCTAGAGAAGTTTCAGGGAAGGTCAGAAG |
| | AGCTCCTGGCTGTGGTGGGCAGGGCAGGAAACCCCTCCACCTTTACACATGCCC |
| | AGGCAGCACCTCAGGCCCTTTGTGGGGCAGGGAAGCTGAGGCAGTAAGCGGGC |
| | AGGCAGAGCTGGAGGCCTTTCAGGCCCAGCCAGCACTCTGGCCTCCTGCCGCCG |
| | CATTCCACCCCAGCCCCTCACACCACTCGGGAGAGGGACATCCTACGGTCCCAA |
| | GGTCAGGAGGGCAGGGCTGGGGTTGACTCAGGCCCCTCCCAGCTGTGGCCACC |
| | TGGGTGTTGGGAGGGCAGAAGTGCAGGCACCTAGGGCCCCCCATGTGCCCACC |
| | CTGGGAGCTCTCCTTGGAACCCATTCCTGAAATTATTTAAAGGGGTTGGCCGGG |
| | CTCCCACCAGGGCCTGGGTGGGAAGGTACAGGCGTTCCCCCGGGGCCTAGTAC |
| | CCCCGCCGTGGCCTATCCACTCCTCACATCCACACACTGCACCCCCACTCCTGG |
| | GGCAGGGCCACCAGCATCCAGGCGGCCAGCAGGCACCTGAGTGGCTGGGACAA |
| | GGGATCCCCCTTCCCTGTGGTTCTATTATATTATAATTATAATTAAATATGAGAG |
| | CATGCTAA (SEQ ID NO: 99) |
| | >NP_005009.2 programmed cell death protein 1 precursor |
| | [Homo sapiens], amino acid sequence |
| | MQIPQAPWPVVWAVLQLGWRPGWFLDSPDRPWNPPTFSPALLVVTEGDNATFTCS |
| | FSNTSESFVLNWYRMSPSNQTDKLAAFPEDRSQPGQDCRFRVTQLPNGRDFHMSV |
| | VRARRNDSGTYLCGAISLAPKAQIKESLRAELRVTERRAEVPTAHPSPSPRPAGQFQ |
| | TLVVGVVGGLLGSLVLLVWVLAVICSRAARGTIGARRTGQPLKEDPSAVPVFSVDY |
| | GELDFQWREKTPEPPVPCVPEQTEYATIVFPSGMGTSSPARRGSADGPRSAQPLRPE |
| | DGHCSWPL (SEQ ID NO: 100) |
| mScarlet | >KY021423.1 Synthetic construct mScarlet gene, partial |
| | cds, mRNA, nucleic acid sequence |
| | ATGGTGAGCAAGGGCGAGGCAGTGATCAAGGAGTTCATGCGGTTCAAGGTGCA |
| | CATGGAGGGCTCCATGAACGGCCACGAGTTCGAGATCGAGGGCGAGGGCGAGG |
| | GCCGCCCCTACGAGGGCACCCAGACCGCCAAGCTGAAGGTGACCAAGGGTGGC |
| | CCCCTGCCCTTCTCCTGGGACATCCTGTCCCCTCAGTTCATGTACGGCTCCAGGG |
| | CCTTCACCAAGCACCCCGCCGACATCCCCGACTACTATAAGCAGTCCTTCCCCG |
| | AGGGCTTCAAGTGGGAGCGCGTGATGAACTTCGAGGACGGCGGCGCCGTGACC |
| | GTGACCCAGGACACCTCCCTGGAGGACGGCACCCTGATCTACAAGGTGAAGCT |
| | CCGCGGCACCAACTTCCCTCCTGACGGCCCCGTAATGCAGAAGAAGACAATGG |
| | GCTGGGAAGCGTCCACCGAGCGGTTGTACCCCGAGGACGGCGTGCTGAAGGGC |
| | GACATTAAGATGGCCCTGCGCCTGAAGGACGGCGGCCGCTACCTGGCGGACTT |
| | CAAGACCACCTACAAGGCCAAGAAGCCCGTGCAGATGCCCGGCGCCTACAACG |
| | TCGACCGCAAGTTGGACATCACCTCCCACAACGAGGACTACACCGTGGTGGAA |
| | CAGTACGAACGCTCCGAGGGCCGCCACTCCACCGGCGGCATGGACGAGCTGTA |
| | CAAG (SEQ ID NO: 101) |
| | >APD76535.1 mScarlet, partial [synthetic construct], |
| | amino acid sequence |
| | MVSKGEAVIKEFMRFKVHMEGSMNGHEFEIEGEGEGRPYEGTQTAKLKVTKGGPL |
| | PFSWDILSPQFMYGSRAFTKHPADIPDYYKQSFPEGFKWERVMNFEDGGAVTVTQ |
| | DTSLEDGTLIYKVKLRGTNFPPDGPVMQKKTMGWEASTERLYPEDGVLKGDIKMA |
| | LRLKDGGRYLADFKTTYKAKKPVQMPGAYNVDRKLDITSHNEDYTVVEQYERSE |
| | GRHSTGGMDELYK (SEQ ID NO: 102) |
| Nano- | >JQ513379.1 NanoLuc reporter vector pNL1.1.CMV[Nluc/CMV], |
| luciferase | complete sequence, mRNA, nucleic acid sequence |
| | GGCCTAACTGGCCTCAATATTGGCCATTAGCCATATTATTCATTGGTTATATAGC |
| | ATAAATCAATATTGGCTATTGGCCATTGCATACGTTGTATCTATATCATAATATG |
| | TACATTTATATTGGCTCATGTCCAATATGACCGCCATGTTGGCATTGATTATTGA |
| | CTAGTTATTAATAGTAATCAATTACGGGGTCATTAGTTCATAGCCCATATATGG |
| | AGTTCCGCGTTACATAACTTACGGTAAATGGCCCGCCTGGCTGACCGCCCAACG |
| | ACCCCCGCCCATTGACGTCAATAATGACGTATGTTCCCATAGTAACGCCAATAG |
| | GGACTTTCCATTGACGTCAATGGGTGGAGTATTTACGGTAAACTGCCCACTTGG |
| | CAGTACATCAAGTGTATCATATGCCAAGTCCGCCCCCTATTGACGTCAATGACG |
| | GTAAATGGCCCGCCTGGCATTATGCCCAGTACATGACCTTACGGGACTTTCCTA |
| | CTTGGCAGTACATCTACGTATTAGTCATCGCTATTACCATGGTGATGCGGTTTTG |
| | GCAGTACACCAATGGGCGTGGATAGCGGTTTGACTCACGGGGATTTCCAAGTCT |
| | CCACCCCATTGACGTCAATGGGAGTTTGTTTTGGCACCAAAATCAACGGGACTT |
| | TCCAAAATGTCGTAATAACCCCGCCCCGTTGACGCAAATGGGCGGTAGGCGTGT |
| | ACGGTGGGAGGTCTATATAAGCAGAGCTCGTTTAGTGAACCGTCAGATCACTAG |
| | AAGCTTTATTGCGGTAGTTTATCACAGTTAAATTGCTAACGCAGTCAGTGGGCC |
| | TCGGCGGCCAAGCTTGGCAATCCGGTACTGTTGGTAAAGCCACCATGGTCTTC |
| | ACACTCGAAGATTTCGTTGGGGACTGGCGACAGACAGCCGGCTACAACCT |
| | GGACCAAGTCCTTGAACAGGGAGGTGTGTCCAGTTTGTTTCAGAATCTCGG |
| | GGTGTCCGTAACTCCGATCCAAAGGATTGTCCTGAGCGGTGAAAATGGGC |
| | TGAAGATCGACATCCATGTCATCATCCCGTATGAAGGTCTGAGCGGCGACC |
| | AAATGGGCCAGATCGAAAAAATTTTTAAGGTGGTGTACCCTGTGGATGATC |
| | ATCACTTTAAGGTGATCCTGCACTATGGCACACTGGTAATCGACGGGGTTA |
| | CGCCGAACATGATCGACTATTTCGGACGGCCGTATGAAGGCATCGCCGTG |
| | TTCGACGGCAAAAAGATCACTGTAACAGGGACCCTGTGGAACGGCAACAA |
| | AATTATCGACGAGCGCCTGATCAACCCCGACGGCTCCCTGCTGTTCCGAGT |
| | AACCATCAACGGAGTGACCGGCTGGCGGCTGTGCGAACGCATTCTGGCGT |
| | AATTCTAGAGTCGGGGCGGCCGGCCGCTTCGAGCAGACATGATAAGATACATT |
| | GATGAGTTTGGACAAACCACAACTAGAATGCAGTGAAAAAAATGCTTTATTTGT |
| | GAAATTTGTGATGCTATTGCTTTATTTGTAACCATTATAAGCTGCAATAAACAA |
| | GTTAACAACAACAATTGCATTCATTTTATGTTTCAGGTTCAGGGGGAGGTGTGG |
| | GAGGTTTTTTAAAGCAAGTAAAACCTCTACAAATGTGGTAAAATCGATAAGGAT |
| | CCGTCGACCGATGCCCTTGAGAGCCTTCAACCCAGTCAGCTCCTTCCGGTGGGC |
| | GCGGGGCATGACTATCGTCGCCGCACTTATGACTGTCTTCTTTATCATGCAACTC |
| | GTAGGACAGGTGCCGGCAGCGCTCTTCCGCTTCCTCGCTCACTGACTCGCTGCG |
| | CTCGGTCGTTCGGCTGCGGCGAGCGGTATCAGCTCACTCAAAGGCGGTAATACG |
| | GTTATCCACAGAATCAGGGGATAACGCAGGAAAGAACATGTGAGCAAAAGGCC |
| | AGCAAAAGGCCAGGAACCGTAAAAAGGCCGCGTTGCTGGCGTTTTTCCATAGG |
| | CTCCGCCCCCCTGACGAGCATCACAAAAATCGACGCTCAAGTCAGAGGTGGCG |
| | AAACCCGACAGGACTATAAAGATACCAGGCGTTTCCCCCTGGAAGCTCCCTCGT |
| | GCGCTCTCCTGTTCCGACCCTGCCGCTTACCGGATACCTGTCCGCCTTTCTCCCT |
| | TCGGGAAGCGTGGCGCTTTCTCATAGCTCACGCTGTAGGTATCTCAGTTCGGTG |
| | TAGGTCGTTCGCTCCAAGCTGGGCTGTGTGCACGAACCCCCCGTTCAGCCCGAC |
| | CGCTGCGCCTTATCCGGTAACTATCGTCTTGAGTCCAACCCGGTAAGACACGAC |
| | TTATCGCCACTGGCAGCAGCCACTGGTAACAGGATTAGCAGAGCGAGGTATGT |
| | AGGCGGTGCTACAGAGTTCTTGAAGTGGTGGCCTAACTACGGCTACACTAGAA |
| | GAACAGTATTTGGTATCTGCGCTCTGCTGAAGCCAGTTACCTTCGGAAAAAGAG |
| | TTGGTAGCTCTTGATCCGGCAAACAAACCACCGCTGGTAGCGGTGGTTTTTTTG |
| | TTTGCAAGCAGCAGATTACGCGCAGAAAAAAAGGATCTCAAGAAGATCCTTTG |
| | ATCTTTTCTACGGGGTCTGACGCTCAGTGGAACGAAAACTCACGTTAAGGGATT |
| | TTGGTCATGAGATTATCAAAAAGGATCTTCACCTAGATCCTTTTAAATTAAAAA |
| | TGAAGTTTTAAATCAATCTAAAGTATATATGAGTAAACTTGGTCTGACAGCGGC |
| | CGCAAATGCTAAACCACTGCAGTGGTTACCAGTGCTTGATCAGTGAGGCACCGA |
| | TCTCAGCGATCTGCCTATTTCGTTCGTCCATAGTGGCCTGACTCCCCGTCGTGTA |
| | GATCACTACGATTCGTGAGGGCTTACCATCAGGCCCCAGCGCAGCAATGATGCC |
| | GCGAGAGCCGCGTTCACCGGCCCCCGATTTGTCAGCAATGAACCAGCCAGCAG |
| | GGAGGGCCGAGCGAAGAAGTGGTCCTGCTACTTTGTCCGCCTCCATCCAGTCTA |
| | TGAGCTGCTGTCGTGATGCTAGAGTAAGAAGTTCGCCAGTGAGTAGTTTCCGAA |
| | GAGTTGTGGCCATTGCTACTGGCATCGTGGTATCACGCTCGTCGTTCGGTATGG |
| | CTTCGTTCAACTCTGGTTCCCAGCGGTCAAGCCGGGTCACATGATCACCCATAT |
| | TATGAAGAAATGCAGTCAGCTCCTTAGGGCCTCCGATCGTTGTCAGAAGTAAGT |
| | TGGCCGCGGTGTTGTCGCTCATGGTAATGGCAGCACTACACAATTCTCTTACCG |
| | TCATGCCATCCGTAAGATGCTTTTCCGTGACCGGCGAGTACTCAACCAAGTCGT |
| | TTTGTGAGTAGTGTATACGGCGACCAAGCTGCTCTTGCCCGGCGTCTATACGGG |
| | ACAACACCGCGCCACATAGCAGTACTTTGAAAGTGCTCATCATCGGGAATCGTT |
| | CTTCGGGGCGGAAAGACTCAAGGATCTTGCCGCTATTGAGATCCAGTTCGATAT |
| | AGCCCACTCTTGCACCCAGTTGATCTTCAGCATCTTTTACTTTCACCAGCGTTTC |
| | GGGGTGTGCAAAAACAGGCAAGCAAAATGCCGCAAAGAAGGGAATGAGTGCG |
| | ACACGAAAATGTTGGATGCTCATACTCGTCCTTTTTCAATATTATTGAAGCATTT |
| | ATCAGGGTTACTAGTACGTCTCTCAAGGATAAGTAAGTAATATTAAGGTACGGG |
| | AGGTATTGGACAGGCCGCAATAAAATATCTTTATTTTCATTACATCTGTGTGTTG |
| | GTTTTTTGTGTGAATCGATAGTACTAACATACGCTCTCCATCAAAACAAAACGA |
| | AACAAAACAAACTAGCAAAATAGGCTGTCCCCAGTGCAAGTGCAGGTGCCAGA |
| | ACATTTCTCT (SEQ ID NO: 103) |
| | >AFJ15599.1 NanoLuc luciferase [NanoLuc reporter vector |
| | pNL1.1.CMV[Nluc/CMV]], amino acid sequence |
| | MVFTLEDFVGDWRQTAGYNLDQVLEQGGVSSLFQNLGVSVTPIQRIVLSGENGLKI |
| | DIHVIIPYEGLSGDQMGQIEKIFKVVYPVDDHHFKVILHYGTLVIDGVTPNMIDYFG |
| | RPYEGIAVFDGKKITVTGTLWNGNKIIDERLINPDGSLLFRVTINGVTGWRLCERILA |
| | (SEQ ID NO: 104) |

TABLE 3B
Proteins of Interest compatible with a Type II membrane anchoring domain
(i.e. a type II vesicle targeting domain)

| | |
|---|---|
| Human 4- | >NM_003811.4 Homo sapiens TNF superfamily member 9 (TNFSF9), |
| 1BBL | mRNA, nucleic acid sequence |
| (CD137L, | AGTCTCTCGTCATGGAATACGCCTCTGACGCTTCACTGGACCCCGAAGCCCCGT |
| TNFSF9) | GGCCTCCCGCGCCCCGCGCTCGCGCCTGCCGCGTACTGCCTTGGGCCCTGGTCG |
| Bold: amino | CGGGGCTGCTGCTGCTGCTGCTGCTCGCTGCCGCCTGCGCCGTCTTCCTCGCCTG |
| acids 71-254 | CCCCTGGGCCGTGTCCGGGGCTCGCGCCTCGCCCGGCTCCGCGGCCAGCCCGAG |
| Underlined: | ACTCCGCGAGGGTCCCGAGCTTTCGCCCGACGATCCCGCCGGCCTCTTGGA |
| amino acids | CCTGCGGCAGGGCATGTTTGCGCAGCTGGTGGCCCAAAATGTTCTGCTGA |
| 85-240 | TCGATGGGCCCCTGAGCTGGTACAGTGACCCAGGCCTGGCAGGCGTGTCC |
| | CTGACGGGGGGCCTGAGCTACAAAGAGGACACGAAGGAGCTGGTGGTGGC |
| | CAAGGCTGGAGTCTACTATGTCTTCTTTCAACTAGAGCTGCGGCGCGTGGT |
| | GGCCGGCGAGGGCTCAGGCTCCGTTTCACTTGCGCTGCACCTGCAGCCAC |
| | TGCGCTCTGCTGCTGGGGCCGCCGCCCTGGCTTTGACCGTGGACCTGCCA |
| | CCCGCCTCCTCCGAGGCTCGGAACTCGGCCTTCGGTTTCCAGGGCCGCTT |
| | GCTGCACCTGAGTGCCGGCCAGCGCCTGGGCGTCCATCTTCACACTGAGG |
| | CCAGGGCACGCCATGCCTGGCAGCTTACCCAGGGCGCCACAGTCTTGGGA |
| | CTCTTCCGGGTGACCCCCGAAATCCCAGCCGGACTCCCTTCACCGAGGTCG |
| | GAATAACGTCCAGCCTGGGTGCAGCCCACCTGGACAGAGTCCGAATCCTACTCC |
| | ATCCTTCATGGAGACCCCTGGTGCTGGGTCCCTGCTGCTTTCTCTACCTCAAGGG |
| | GCTTGGCAGGGGTCCCTGCTGCTGACCTCCCCTTGAGGACCCTCCTCACCCACT |
| | CCTTCCCCAAGTTGGACCTTGATATTTATTCTGAGCCTGAGCTCAGATAATATAT |
| | TATATATATTATATATATATATATATTTCTATTTAAAGAGGATCCTGAGTTTGTG |
| | AATGGACTTTTTTAGAGGAGTTGTTTTGGGGGGGGGGGGGTCTTCGACATTGCC |
| | GAGGCTGGTCTTGAACTCCTGGACTTAGACGATCCTCCTGCCTCAGCCTCCCAA |
| | GCAACTGGGATTCATCCTTTCTATTAATTCATTGTACTTATTTGCTTATTTGTGTG |
| | TATTGAGCATCTGTAATGTGCCAGCATTGTGCCCAGGCTAGGGGGCTATAGAAA |
| | CATCTAGAAATAGACTGAAAGAAAATCTGAGTTATGGTAATACGTGAGGAATT |
| | TAAAGACTCATCCCCAGCCTCCACCTCCTGTGTGATACTTGGGGGCTAGCTTTTT |
| | TCTTTCTTTCTTTTTTTTGAGATGGTCTTGTTCTGTCAACCAGGCTAGAATGCAG |
| | CGGTGCAATCATGAGTCAATGCAGCCTCCAGCCTCGACCTCCCGAGGCTCAGGT |
| | GATCCTCCCATCTCAGCCTCTCGAGTAGCTGGGACCACAGTTGTGTGCCACCAC |
| | ACTTGGCTAACTTTTTAATTTTTTTGCGGAGACGGTATTGCTATGTTGCCAAGGT |
| | TGTTTACATGCCAGTACAATTTATAATAAACACTCATTTTTCCTCCC (SEQ ID |
| | NO: 105) |
| | >NP_003802.1 tumor necrosis factor ligand superfamily member 9 |
| | [Homo sapiens], amino acid sequence |
| | MEYASDASLDPEAPWPPAPRARACRVLPWALVAGLLLLLLLAAACAVFLACPWA |
| | VSGARASPGSAASPRLREGPELSPDDPAGLLDLROGMFAQLVAQNVLLIDGPLS |
| | WYSDPGLAGVSLTGGLSYKEDTKELVVAKAGVYYVFFQLELRRVVAGEGSGS |
| | VSLALHLQPLRSAAGAAALALTVDLPPASSEARNSAFGFQGRLLHLSAGQRLG |
| | VHLHTEARARHAWQLTQGATVLGLFRVTPEIPAGLPSPRSE (SEQ ID NO: 106) |
| Human | >NM_005092.4 Homo sapiens TNF superfamily member 18 (TNFSF18), |
| GITRL | mRNA, nucleic acid sequence |
| (TNFSF18) | ATCACTTGTGAATTTTTGTTTTCCACAGCTCTCATTTCTCCAAAAATGTGTTTGA |
| Bold: amino | GCCACTTGGAAAATATGCCTTTAAGCCATTCAAGAACTCAAGGAGCTCAGAGAT |
| acids 56-177 | CATCCTGGAAGCTGTGGCTCTTTTGCTCAATAGTTATGTTGCTATTTCTTTGCTC |
| | CTTCAGTTGGCTAATCTTTATTTTTCTCCAATTAGAGACTGCTAAGGAGCCCTG |
| | TATGGCTAAGTTTGGACCATTACCCTCAAAATGGCAAATGGCATCTTCTGA |
| | ACCTCCTTGCGTGAATAAGGTGTCTGACTGGAAGCTGGAGATACTTCAGAA |
| | TGGCTTATATTTAATTTATGGCCAAGTGGCTCCCAATGCAAACTACAATGA |
| | TGTAGCTCCTTTTGAGGTGCGGCTGTATAAAAACAAAGACATGATACAAAC |
| | TCTAACAAACAAATCTAAAATCCAAAATGTAGGAGGGACTTATGAATTGCA |
| | TGTTGGGGACACCATAGACTTGATATTCAACTCTGAGCATCAGGTTCTAAA |
| | AAATAATACATACTGGGGTATCATTTTACTAGCAAATCCCCAATTCATCTCC |
| | TAGAGACTTGATTTGATCTCCTCATTCCCTTCAGCACATGTAGAGGTGCCAGTG |
| | GGTGGATTGGAGGGAGAAGATATTCAATTTCTAGAGTTTGTCTGTCTACAAAAA |
| | TCAACACAAACAGAACTCCTCTGCACGTGAATTTTCATCTATCATGCCTATCTG |
| | AAAGAGACTCAGGGGAAGAGCCAAAGACTTTTGGTTGGATCTGCAGAGATACT |
| | TCATTAATCCATGATAAAACAAATATGGATGACAGAGGACATGTGCTTTTCAAA |
| | GAATCTTTATCTAATTCTTGAATTCATGAGTGGAAAAATGGAGTTCTATTCCCAT |
| | GGAAGATTTACCTGGTATGCAAAAAGGATCTGGGGCAGTAGCCTGGCTTTGTTC |
| | TCATATTCTTGGGCTGCTGTAATTCATTCTTCTCATACTCCCATCTTCTGAGACC |
| | CTCCCAATAAAAAGTAGACTGATAGGATGGCCACAGATATGCCTACCATACCCT |
| | ACTTTAGATATGGTGGTGTTAGAAGATAAAGAACAATCTGAGAACTATTGGAAT |
| | AGAGGTACAAGTGGCATAAAATGGAATGTACGCTATCTGGAAATTTCTCTTGGT |
| | TTTATCTTCCTCAGGATGCAGGGTGCTTTAAAAAGCCTTATCAAAGGAGTCATT |
| | CCGAACCCTCACGTAGAGCTTTGTGAGACCTTACTGTTGGTGTGTGTGTCTAAA |
| | CATTGCTAATTGTAAAGAAAGAGTAACCATTAGTAATCATTAGGTTTAACCCCA |
| | GAATGGTATTATCATTACTGGATTATGTCATGTAATGATTTAGTATTTTTAGCTA |
| | GCTTTCCACAGTTTGCAAAGTGCTTTCGTAAAACAGTTAGCAATTCTATGAAGT |
| | TAATTGGGCAGGCATTTGGGGGAAAATTTTAGTGATGAGAATGTGATAGCATA |
| | GCATAGCCAACTTTCCTCAACTCATAGGACAAGTGACTACAAGAGGCAATGGG |
| | TAGTCCCCTGCATTGCACTGTCTCAGCTTTAGAATTGTTATTTCTGCTATCGTGT |
| | TATAAGACTCTAAAACTTAGCGAATTCACTTTTCAGGAAGCATATTCCCCTTTA |
| | GCCCAAGGTGAGCAGAGTGAAGCTACAACAGATCTTTCCTTTACCAGCACACTT |
| | TTTTTTTTTTTCCTGCCTGAATCAGGGAGATCCAGGATGCTGTTCAGGCCTTATC |
| | CCAACCAAATTCCCCTCTTCACTTTGCAGGGCCCATCTTAGTCAAATGTGCTAAC |
| | TTCTAAAATAATAAATAGCACTAATTCAAAATTTTTGGACTCTTAAATTAGCTA |
| | CTTGCAGGTTCTTGTTGAAAGGTATATAATATTACATTGTAAACAAATTTAAAA |
| | TATTTATGGATATTTGTGAAAAGCTGCATTATGTTAAATAATATTACATGTAAA |
| | GCTATTTAAAAGAGGTTTTTTTTGTATTTTGTTTAACAAAAATTGCTCAGGAGCA |
| | TGCTAAGCCTGAGGCCAAGTTGTTTCTTAGTATGACTTTTTAAAAAAACATCTG |
| | CTGAGTAGCTACAGGGCCAAAGACTTGGAGAGCTTGTTTCTGTTGCATTTGCAT |
| | ATCTTCTCAGGAAATTAAAGTGTGTCATACATATGTGTGTGTGTGTGTGTGTGTG |
| | TGTGTATATGTGTGTGTGTATATATATGTATACTTATAAAATCTTGGTGTTCTTG |
| | ATCTTTGTTGTGTTATAAGCAATGTGTGCTGGAGTGGGCTGGTGCTAGCTTATA |
| | AGCACATATTATTAAATTTTCAGGAATGTTGCACTTTAGTTATTAACTATAGGCA |
| | TTCTTGAAATTGGCTATGGTGGGAGTATTTATACCATGTAAATTGGCAAACACT |
| | ACACATTTTCCTTTTGGACAGCTAGTTCACCAGCACACCACTGTGAAACTCTCCT |
| | TAATGACTCCTCTCTGCCCCCGCTTCATTCCTGGGATAATCATAGCAGACTAAG |
| | GGAGAAAATGAAATTGTAAAAATTTGGCATACTGGTGATTTCTCAGGGCAAGC |
| | AGAGGTTACTACAGCTGCAGCTAGAGGGATGACTACCAACAGGTGACCTTTAC |
| | ATTTTCCTGATGTTATAATTTTAGCTTTTGTTTTCAATGTATACTGTTTTCCTGTT |
| | TCTCCACATAGTAGTCTGCATTTTAAATCTATAATAAAACATGCTGATAACTGG |
| | (SEQ ID NO: 107) |
| | >NP 005083.3 tumor necrosis factor ligand superfamily member |
| | 18 [Homo sapiens], amino acid sequence |
| | MCLSHLENMPLSHSRTQGAQRSSWKLWLFCSIVMLLFLCSFSWLIFIFLQLETAKEP |
| | CMAKFGPLPSKWQMASSEPPCVNKVSDWKLEILQNGLYLIYGQVAPNANYND |
| | VAPFEVRLYKNKDMIQTLTNKSKIQNVGGTYELHVGDTIDLIFNSEHQVLKNN |
| | TYWGIILLANPQFIS (SEQ ID NO: 108) |
| Human | >NM_003326.5 Homo sapiens TNF superfamily member 4 (TNFSF4), |
| OX40L | transcript variant 1, mRNA, nucleic acid sequence |
| (TNFSF4) | CAATCGCCTTTTATCTCTGGCCCTGGGACCTTTGCCTATTTTCTGATTGATAGGC |
| Bold: amino | TTTGTTTTGTCTTTACCTCCTTCTTTCTGGGGAAAACTTCAGTTTTATCGCACGTT |
| acids 54-183 | CCCCTTTTCCATATCTTCATCTTCCCTCTACCCAGATTGTGAAGATGGAAAGGGT |
| | CCAACCCCTGGAAGAGAATGTGGGAAATGCAGCCAGGCCAAGATTCGAGAGGA |
| | ACAAGCTATTGCTGGTGGCCTCTGTAATTCAGGGACTGGGGCTGCTCCTGTGCT |
| | TCACCTACATCTGCCTGCACTTCTCTGCTCTTCAGGTATCACATCGGTATCCTC |
| | GAATTCAAAGTATCAAAGTACAATTTACCGAATATAAGAAGGAGAAAGGTT |
| | TCATCCTCACTTCCCAAAAGGAGGATGAAATCATGAAGGTGCAGAACAACT |
| | CAGTCATCATCAACTGTGATGGGTTTTATCTCATCTCCCTGAAGGGCTACT |
| | TCTCCCAGGAAGTCAACATTAGCCTTCATTACCAGAAGGATGAGGAGCCCC |
| | TCTTCCAACTGAAGAAGGTCAGGTCTGTCAACTCCTTGATGGTGGCCTCTC |
| | TGACTTACAAAGACAAAGTCTACTTGAATGTGACCACTGACAATACCTCCC |
| | TGGATGACTTCCATGTGAATGGCGGAGAACTGATTCTTATCCATCAAAATC |
| | CTGGTGAATTCTGTGTCCTTTGAGGGGCTGATGGCAATATCTAAAACCAGGC |
| | ACCAGCATGAACACCAAGCTGGGGGTGGACAGGGCATGGATTCTTCATTGCAA |
| | GTGAAGGAGCCTCCCAGCTCAGCCACGTGGGATGTGACAAGAAGCAGATCCTG |
| | GCCCTCCCGCCCCCACCCCTCAGGGATATTTAAAACTTATTTTATATACCAGTTA |
| | ATCTTATTTATCCTTATATTTTCTAAATTGCCTAGCCGTCACACCCCAAGATTGC |
| | CTTGAGCCTACTAGGCACCTTTGTGAGAAAGAAAAAATAGATGCCTCTTCTTCA |
| | AGATGCATTGTTTCTATTGGTCAGGCAATTGTCATAATAAACTTATGTCATTGAA |
| | AACGGTACCTGACTACCATTTGCTGGAAATTTGACATGTGTGTGGCATTATCAA |
| | AATGAAGAGGAGCAAGGAGTGAAGGAGTGGGGTTATGAATCTGCCAAAGGTG |
| | GTATGAACCAACCCCTGGAAGCCAAAGCGGCCTCTCCAAGGTTAAATTGATTGC |
| | AGTTTGCATATTGCCTAAATTTAAACTTTCTCATTTGGTGGGGGTTCAAAAGAA |
| | GAATCAGCTTGTGAAAAATCAGGACTTGAAGAGAGCCGTCTAAGAAATACCAC |
| | GTGCTTTTTTTCTTTACCATTTTGCTTTCCCAGCCTCCAAACATAGTTAATAGAA |
| | ATTTCCCTTCAAAGAACTGTCTGGGGATGTGATGCTTTGAAAAATCTAATCAGT |
| | GACTTAAGAGAGATTTTCTTGTATACAGGGAGAGTGAGATAACTTATTGTGAAG |
| | GGTTAGCTTTACTGTACAGGATAGCAGGGAACTGGACATCTCAGGGTAAAAGT |
| | CAGTACGGATTTTAATAGCCTGGGGAGGAAAACACATTCTTTGCCACAGACAG |
| | GCAAAGCAACACATGCTCATCCTCCTGCCTATGCTGAGATACGCACTCAGCTCC |
| | ATGTCTTGTACACACAGAAACATTGCTGGTTTCAAGAAATGAGGTGATCCTATT |
| | ATCAAATTCAATCTGATGTCAAATAGCACTAAGAAGTTATTGTGCCTTATGAAA |
| | AATAATGATCTCTGTCTAGAAATACCATAGACCATATATAGTCTCACATTGATA |
| | ATTGAAACTAGAAGGGTCTATAATCAGCCTATGCCAGGGCTTCAATGGAATAGT |
| | ATCCCCTTATGTTTAGTTGAAATGTCCCCTTAACTTGATATAATGTGTTATGCTT |
| | ATGGCGCTGTGGACAATCTGATTTTTCATGTCAACTTTCCAGATGATTTGTAACT |
| | TCTCTGTGCCAAACCTTTTATAAACATAAATTTTTGAGATATGTATTTTAAAATT |
| | GTAGCACATGTTTCCCTGACATTTTCAATAGAGGATACAACATCACAGAATCTT |
| | TCTGGATGATTCTGTGTTATCAAGGAATTGTACTGTGCTACAATTATCTCTAGAA |
| | TCTCCAGAAAGGTGGAGGGCTGTTCGCCCTTACACTAAATGGTCTCAGTTGGAT |
| | TTTTTTTTCCTGTTTTCTATTTCCTCTTAAGTACACCTTCAACTATATTCCCATCC |
| | CTCTATTTTAATCTGTTATGAAGGAAGGTAAATAAAAATGCTAAATAGAAGAAA |
| | TTGTAGGTAAGGTAAGAGGAATCAAGTTCTGAGTGGCTGCCAAGGCACTCACA |
| | GAATCATAATCATGGCTAAATATTTATGGAGGGCCTACTGTGGACCAGGCACTG |
| | GGCTAAATACTTACATTTACAAGAATCATTCTGAGACAGATATTCAATGATATC |
| | TGGCTTCACTACTCAGAAGATTGTGTGTGTGTTTGTGTGTGTGTGTGTGTGTGTA |
| | TTTCACTTTTTGTTATTGACCATGTTCTGCAAAATTGCAGTTACTCAGTGAGTGA |
| | TATCCGAAAAAGTAAACGTTTATGACTATAGGTAATATTTAAGAAAATGCATGG |
| | TTCATTTTTAAGTTTGGAATTTTTATCTATATTTCTCACAGATGTGCAGTGCACA |
| | TGCAGGCCTAAGTATATGTTGTGTGTGTTGTTTGTCTTTGATGTCATGGTCCCCT |
| | CTCTTAGGTGCTCACTCGCTTTGGGTGCACCTGGCCTGCTCTTCCCATGTTGGCC |
| | TCTGCAACCACACAGGGATATTTCTGCTATGCACCAGCCTCACTCCACCTTCCTT |
| | CCATCAAAAATATGTGTGTGTGTCTCAGTCCCTGTAAGTCATGTCCTTCACAGG |
| | GAGAATTAACCCTTCGATATACATGGCAGAGTTTTGTGGGAAAAGAATTGAATG |
| | AAAAGTCAGGAGATCAGAATTTTAAATTTGACTTAGCCACTAACTAGCCATGTA |
| | ACCTTGGGAAAGTCATTTCCCATTTCTGGGTCTTGCTTTTCTTTCTGTTAAATGA |
| | GAGGAATGTTAAATATCTAACAGTTTAGAATCTTATGCTTACAGTGTTATCTGT |
| | GAATGCACATATTAAATGTCTATGTTCTTGTTGCTATGAGTCAAGGAGTGTAAC |
| | CTTCTCCTTTACTATGTTGAATGTATTTTTTTCTGGACAAGCTTACATCTTCCTCA |
| | GCCATCTTTGTGAGTCCTTCAAGAGCAGTTATCAATTGTTAGTTAGATATTTTCT |
| | ATTTAGAGAATGCTTAAGGGATTCCAATCCCGATCCAAATCATAATTTGTTCTT |
| | AAGTATACTGGGCAGGTCCCCTATTTTAAGTCATAATTTTGTATTTAGTGCTTTC |
| | CTGGCTCTCAGAGAGTATTAATATTGATATTAATAATATAGTTAATAGTAATAT |
| | TGCTATTTACATGGAAACAAATAAAAGATCTCAGAATTCACTA (SEQ ID NO: |
| | 109) |
| | >NP_003317.1 tumor necrosis factor ligand superfamily member 4 |
| | isoform 1 [Homo sapiens], amino acid sequence |
| | MERVQPLEENVGNAARPRFERNKLLLVASVIQGLGLLLCFTYICLHFSALQVSHRY |
| | PRIQSIKVQFTEYKKEKGFILTSQKEDEIMKVQNNSVIINCDGFYLISLKGYFSQ |
| | EVNISLHYQKDEEPLFQLKKVRSVNSLMVASLTYKDKVYLNVTTDNTSLDDFH |
| | VNGGELILIHQNPGEFCVL (SEQ ID NO: 110) |
| Human | >NM_001252.5 Homo sapiens CD70 molecule (CD70), transcript |
| CD27L | variant 1, mRNA, nucleic acid sequence |
| (CD70) | AGAGAGGGGCAGGCTGGTCCCCTGACAGGTTGAAGCAAGTAGACGCCCAGGAG |
| Bold: amino | CCCCGGGAGGGGGCTGCAGTTTCCTTCCTTCCTTCTCGGCAGCGCTCCGCGCCC |
| acids 53-193 | CCATCGCCCCTCCTGCGCTAGCGGAGGTGATCGCCGCGGCGATGCCGGAGGAG |
| | GGTTCGGGCTGCTCGGTGCGGCGCAGGCCCTATGGGTGCGTCCTGCGGGCTGCT |
| | TTGGTCCCATTGGTCGCGGGCTTGGTGATCTGCCTCGTGGTGTGCATCCAGCGCT |
| | TCGCACAGGCTCAGCAGCAGCTGCCGCTCGAGTCACTTGGGTGGGACGTAGC |
| | TGAGCTGCAGCTGAATCACACAGGACCTCAGCAGGACCCCAGGCTATACT |
| | GGCAGGGGGGCCCAGCACTGGGCCGCTCCTTCCTGCATGGACCAGAGCTG |
| | GACAAGGGGCAGCTACGTATCCATCGTGATGGCATCTACATGGTACACATC |
| | CAGGTGACGCTGGCCATCTGCTCCTCCACGACGGCCTCCAGGCACCACCC |
| | CACCACCCTGGCCGTGGGAATCTGCTCTCCCGCCTCCCGTAGCATCAGCCT |
| | GCTGCGTCTCAGCTTCCACCAAGGTTGTACCATTGCCTCCCAGCGCCTGAC |
| | GCCCCTGGCCCGAGGGGACACACTCTGCACCAACCTCACTGGGACACTTTT |
| | GCCTTCCCGAAACACTGATGAGACCTTCTTTGGAGTGCAGTGGGTGCGCCC |
| | CTGACCACTGCTGCTGATTAGGGTTTTTTAAATTTTATTTTATTTTATTTAAGTTC |
| | AAGAGAAAAAGTGTACACACAGGGGCCACCCGGGGTTGGGGTGGGAGTGTGGT |
| | GGGGGGTAGTGGTGGCAGGACAAGAGAAGGCATTGAGCTTTTTCTTTCATTTTC |
| | CTATTAAAAAATACAAAAATCA (SEQ ID NO: 111) |
| | >NP_001243.1 CD70 antigen isoform 1 [Homo sapiens], amino acid sequence |
| | MPEEGSGCSVRRRPYGCVLRAALVPLVAGLVICLVVCIQRFAQAQQQLPLESLGW |
| | DVAELQLNHTGPQQDPRLYWQGGPALGRSFLHGPELDKGQLRIHRDGIYMV |
| | HIQVTLAICSSTTASRHHPTTLAVGICSPASRSISLLRLSFHQGCTIASQRLTPLA |
| | RGDTLCTNLTGTLLPSRNTDETFFGVQWVRP (SEQ ID NO: 112) |
| Human | >NM_001244.4 Homo sapiens TNF superfamily member 8 (TNFSF8), |
| CD30L | transcript variant 1, mRNA, nucleic acid sequence |
| (TNFSF8) | GTCATTTTCCTACGCGCCCTCTGACATCAGCCACCTTCTCTGTAGCTAGTTTCTC |
| Bold: amino | TGCACACAACTTAATCCCTGGCAATGAAAAATGAACCTCTCCCCCACCCTTGCT |
| acids 99-234 | GCCGCCTCTCGCCTCACGCCCCCAGAGAAGAGTTTCTCCACCAGGCAGCAGGTG |
| | AAGGTTTTTTTCCAAGTCACATGATTCAGGATTCAGGGGGAGAATCCTTCTTGG |
| | AACAGAGATGGGCCCAGAACTGAATCAGATGAAGAGAGATAAGGTGTGATGTG |
| | GGGAAGACTATATAAAGAATGGACCCAGGGCTGCAGCAAGCACTCAACGGAAT |
| | GGCCCCTCCTGGAGACACAGCCATGCATGTGCCGGCGGGCTCCGTGGCCAGCC |
| | ACCTGGGGACCACGAGCCGCAGCTATTTCTATTTGACCACAGCCACTCTGGCTC |
| | TGTGCCTTGTCTTCACGGTGGCCACTATTATGGTGTTGGTCGTTCAGAGGACGG |
| | ACTCCATTCCCAACTCACCTGACAACGTCCCCCTCAAAGGAGGAAATTGCTCAG |
| | AAGACCTCTTATGTATCCTGAAAAGGGCTCCATTCAAGAAGTCATGGGCCTAC |
| | CTCCAAGTGGCAAAGCATCTAAACAAAACCAAGTTGTCTTGGAACAAAGAT |
| | GGCATTCTCCATGGAGTCAGATATCAGGATGGGAATCTGGTGATCCAATTC |
| | CCTGGTTTGTACTTCATCATTTGCCAACTGCAGTTTCTTGTACAATGCCCAA |
| | ATAATTCTGTCGATCTGAAGTTGGAGCTTCTCATCAACAAGCATATCAAAA |
| | AACAGGCCCTGGTGACAGTGTGTGAGTCTGGAATGCAAACGAAACACGTA |
| | TACCAGAATCTCTCTCAATTCTTGCTGGATTACCTGCAGGTCAACACCACC |
| | ATATCAGTCAATGTGGATACATTCCAGTACATAGATACAAGCACCTTTCCT |
| | CTTGAGAATGTGTTGTCCATCTTCTTATACAGTAATTCAGACTGAACAGTTT |
| | CTCTTGGCCTTCAGGAAGAAAGCGCCTCTCTACCATACAGTATTTCATCCCTCCA |
| | AACACTTGGGCAAAAAGAAAACTTTAGACCAAGACAAACTACACAGGGTATTA |
| | AATAGTATACTTCTCCTTCTGTCTCTTGGAAAGATACAGCTCCAGGGTTAAAAA |
| | GAGAGTTTTTAGTGAAGTATCTTTCAGATAGCAGGCAGGGAAGCAATGTAGTGT |
| | GGTGGGCAGAGCCCCACACAGAATCAGAAGGGATGAATGGATGTCCCAGCCCA |
| | ACCTCTAATTCACTGTATGGTCTTGATCTATTTCTTCTGTTTTGAGAGCCTCCAG |
| | TTAAAATGGGGCTCCAGTACCAGAGCAGCTAGCAACTCTGCCCTAATGGGAAA |
| | TGAAGGGGAGCTGGGTGTGAGTGTTTACACTGTGCCCTTCACGGGATACTTCTT |
| | TTATCTGCAGATGGCCTAATACTTAGTTGTCCAAGTCGCGATCAAGGACTCTCT |
| | CACACAGGAAACTTCCCTATACTGGCAGATACACTTGTGACTGAACCATGCCCA |
| | GTTTATGCCTGTCTGACTGTCACTCTGGCACTAGGAGGCTGATCTTGTACTCCAT |
| | ATGACCCCACCCCTAGGAACCCCCAGGGAAAACCAGGCTGGGACAGCCCCCTG |
| | TTCCTGAGATGGAAAGCACAAATTTAATACACCACCACAATGGAAAACAAGTT |
| | CAAAGACTTTTACTTACAGATCCTGGACAGAAAGGGCATAATGAGTCTGAAGG |
| | GCAGTCCTCCTTCTCTAGGTTACATGAGGCAGGAATAAGAAGTCAGACAGAGA |
| | CAGCAAGACAGTTAACAATGTAGGTAAAGAAATAGGGTGTGGTCACTCTCAAT |
| | TCACTGGCAAATGCCTGAATGGTCTGTCTGAAGGAAGCAACAGAGAAGTGGGG |
| | AATCCAGTCTGCTAGGCAGGAAAGATGCCTCTAAGTTCTTGTCTCTGGCCAGAG |
| | GTGTGGTATAGAACCAGAAACCCATATCAAGGGTGACTAAGCCCGGCTTCTGGT |
| | ATGAGAAATTAAACTTGTATACAAAATGGTTGCCAAGGCAACATAAAATTATA |
| | AGAATTCACTATACCTTCCCCTCCCTGGAACTCAGGATCCAAGTCTAGAAAATG |
| | AAAGGACTGGGTTTGAATTGCTTCAAAACCTCTTCCATCTCAGAAGACCAGACC |
| | CTGGGAACTGAGATTCCAGACACAATTTTGGAAGCTCTCCAACCAAAATAAGG |
| | CCCCCCTACCCCAGTATATAATTGAAGACACTAGTAACACCTGACTGCATCTCA |
| | TCTCAGCAGAGCCAGAATATGGGGACAAGGTTCAGGGTGCCCTGCTGAATGGT |
| | GTGAACAGCAGGATCTCAAGGATGTAATGGAAAGAACTACCACACTGACCATC |
| | CAGAATCTAAGAGACCATCTGGGTGTTTGGGAAACCATCTGACGAGGCCTGACT |
| | CTATTCCAGTTAGATTGACAATAATTGAGCAGCAGGCATTTTTCATTTCTGGTCA |
| | GGAAAGCATTGTGCCTTTAGCAAACAATCAGTGTGCAACAGTGATGTGGTCATC |
| | TAGCCAGGGAATGGCTGCTCCATCCCCTGCATAATATATTCCTGCTTCAAACAC |
| | CTCTCAGAAAACCAGTTCCGCGAGGGTTTTTATATCCCCACAAAGTTGTTGAGA |
| | GACAATGATGACCCTGGAAGTGGGGAGGAGGACTTCTGAGAAACAGCAACCTC |
| | TCTCCTGATTGGGGTAGCCATGAGATTTCTCTAGCTATATCCAACTTGGCATCTG |
| | TACATCATCTTTGGAGGAACATCTTATTTGTGGAAGGACCTTGACAAGCCGTTT |
| | GAGATGGAATGTAGGCCCTGATGTTATGCTTCAGTAAAAAAAGATGGAAGCTT |
| | CCCTGCTATACCAAAACATGGAGCAAAATTTGCATTTTTCTCAAGAAGGAGAGA |
| | AAAGGAGTAGGACTCCAGCAAAGTTTGTCAGAAGGAAAGCTAGAAAAGATTTA |
| | AAAGAAAAAAAGAAAGAACAAATCAGCAGTGGTGGTATGGATGAAAGGGACT |
| | TGAGAGAACAAAAATGGCTAAGGGAAAATTTTAAGTCATCTGCTGAGCAGTGT |
| | GCTGTGTCAACCTCCTCCTAGGTCTCCTCTATGAAATATTTAGTAAAGTCTACAT |
| | TTCTCTTTAACTCTTTCTGTGAGTAGATTCTTTGGGAGAAGCAGGCATTGGAAG |
| | AGGTGTTGAATTCAGCAAGCCAAATGGTCTGTGGTAAAAAACAAAACAGACTT |
| | TGAGACTCAAGGCTAAAAAAACAGGGAAATGGCTGGCATTTGAGTCACACACT |
| | AACTGCATAGGACAAATGAATCTTGCTTAAACCAACTCATGCATTCTTGAAAAG |
| | GTATATGCAACCCAACTGTGTGTTAACTAAGCAATTTTTTTGCCATCTCACATTC |
| | TAACTCGAGAAAGATTCCATTTTCATTTTTCACCAACTGTTCTCTGAGCAGAGGT |
| | ACCTGACTTTTGCACTGTGAGTGGTTTCTAATCTCAGTCTCTGTCAAGCAATGCT |
| | AAGAAAGCCAACACCTAAAGACACAAGGGGTACATCATTTAAATGAATAATGT |
| | AACCAAACAAACAAAAAAAGAGAATAATCATTAATAACTCAACTGATAGATAT |
| | GTAGGGAGTAGGCAACCCAGGAAGTTTAAAACTAAATTCTGTTACTCTTGAGGG |
| | TTAACCAGCCCCTGGGAATGTTATGAGCAAATGATACTCCATGAGTAAAATGAT |
| | ATCTATGCAAGTAAAATAAATAATTTATCTAACTGGGAA (SEQ ID NO: 113) |
| | >NP_001235.1 tumor necrosis factor ligand superfamily member |
| | 8 isoform 1 [Homo sapiens], amino acid sequence |
| | MDPGLQQALNGMAPPGDTAMHVPAGSVASHLGTTSRSYFYLTTATLALCLVFTVA |
| | TIMVLVVQRTDSIPNSPDNVPLKGGNCSEDLLCILKRAPFKKSWAYLQVAKHLNK |
| | TKLSWNKDGILHGVRYQDGNLVIQFPGLYFIICQLQFLVQCPNNSVDLKLELLI |
| | NKHIKKQALVTVCESGMQTKHVYQNLSQFLLDYLQVNTTISVNVDTFQYIDTS |
| | TFPLENVLSIFLYSNSD (SEQ ID NO: 114) |
| Human | >NM_000074.3 Homo sapiens CD40 ligand (CD40LG), mRNA, |
| CD40L | nucleic acid sequence |
| Bold: amino | AATCCTGAGTAAGGTGGCCACTTTGACAGTCTTCTCATGCTGCCTCTGCCACCTT |
| acids 117-261 | CTCTGCCAGAAGATACCATTTCAACTTTAACACAGCATGATCGAAACATACAAC |
| | CAAACTTCTCCCCGATCTGCGGCCACTGGACTGCCCATCAGCATGAAAATTTTT |
| | ATGTATTTACTTACTGTTTTTCTTATCACCCAGATGATTGGGTCAGCACTTTTTG |
| | CTGTGTATCTTCATAGAAGGTTGGACAAGATAGAAGATGAAAGGAATCTTCATG |
| | AAGATTTTGTATTCATGAAAACGATACAGAGATGCAACACAGGAGAAAGATCC |
| | TTATCCTTACTGAACTGTGAGGAGATTAAAAGCCAGTTTGAAGGCTTTGTGAAG |
| | GATATAATGTTAAACAAAGAGGAGACGAAGAAAGAAAACAGCTTTGAAATGCA |
| | AAAAGGTGATCAGAATCCTCAAATTGCGGCACATGTCATAAGTGAGGCCAG |
| | CAGTAAAACAACATCTGTGTTACAGTGGGCTGAAAAAGGATACTACACCAT |
| | GAGCAACAACTTGGTAACCCTGGAAAATGGGAAACAGCTGACCGTTAAAA |
| | GACAAGGACTCTATTATATCTATGCCCAAGTCACCTTCTGTTCCAATCGGG |
| | AAGCTTCGAGTCAAGCTCCATTTATAGCCAGCCTCTGCCTAAAGTCCCCCG |
| | GTAGATTCGAGAGAATCTTACTCAGAGCTGCAAATACCCACAGTTCCGCCA |
| | AACCTTGCGGGCAACAATCCATTCACTTGGGAGGAGTATTTGAATTGCAAC |
| | CAGGTGCTTCGGTGTTTGTCAATGTGACTGATCCAAGCCAAGTGAGCCATG |
| | GCACTGGCTTCACGTCCTTTGGCTTACTCAAACTCTGAACAGTGTCACCTTG |
| | CAGGCTGTGGTGGAGCTGACGCTGGGAGTCTTCATAATACAGCACAGCGGTTA |
| | AGCCCACCCCCTGTTAACTGCCTATTTATAACCCTAGGATCCTCCTTATGGAGA |
| | ACTATTTATTATACACTCCAAGGCATGTAGAACTGTAATAAGTGAATTACAGGT |
| | CACATGAAACCAAAACGGGCCCTGCTCCATAAGAGCTTATATATCTGAAGCAG |
| | CAACCCCACTGATGCAGACATCCAGAGAGTCCTATGAAAAGACAAGGCCATTA |
| | TGCACAGGTTGAATTCTGAGTAAACAGCAGATAACTTGCCAAGTTCAGTTTTGT |
| | TTCTTTGCGTGCAGTGTCTTTCCATGGATAATGCATTTGATTTATCAGTGAAGAT |
| | GCAGAAGGGAAATGGGGAGCCTCAGCTCACATTCAGTTATGGTTGACTCTGGGT |
| | TCCTATGGCCTTGTTGGAGGGGGCCAGGCTCTAGAACGTCTAACACAGTGGAGA |
| | ACCGAAACCCCCCCCCCCCCCCCGCCACCCTCTCGGACAGTTATTCATTCTCTTT |
| | CAATCTCTCTCTCTCCATCTCTCTCTTTCAGTCTCTCTCTCTCAACCTCTTTCTTC |
| | CAATCTCTCTTTCTCAATCTCTCTGTTTCCCTTTGTCAGTCTCTTCCCTCCCCCAG |
| | TCTCTCTTCTCAATCCCCCTTTCTAACACACACACACACACACACACACACACA |
| | CACACACACACACACACACACACAGAGTCAGGCCGTTGCTAGTCAGTTCTCTTC |
| | TTTCCACCCTGTCCCTATCTCTACCACTATAGATGAGGGTGAGGAGTAGGGAGT |
| | GCAGCCCTGAGCCTGCCCACTCCTCATTACGAAATGACTGTATTTAAAGGAAAT |
| | CTATTGTATCTACCTGCAGTCTCCATTGTTTCCAGAGTGAACTTGTAATTATCTT |
| | GTTATTTATTTTTTGAATAATAAAGACCTCTTAACATTA (SEQ ID NO: 115) |
| | >NP_000065.1 CD40 ligand [Homo sapiens], amino acid sequence |
| | MIETYNQTSPRSAATGLPISMKIFMYLLTVFLITQMIGSALFAVYLHRRLDKIEDERN |
| | LHEDFVFMKTIQRCNTGERSLSLLNCEEIKSQFEGFVKDIMLNKEETKKENSFEMQK |
| | GDQNPQIAAHVISEASSKTTSVLQWAEKGYYTMSNNLVTLENGKQLTVKRQG |
| | LYYIYAQVTFCSNREASSQAPFIASLCLKSPGRFERILLRAANTHSSAKPCGQQS |
| | IHLGGVFELQPGASVFVNVTDPSQVSHGTGFTSFGLLKL (SEQ ID NO: 116) |
| Human | >NM_003807.5 Homo sapiens TNF superfamily member 14 (TNFSF14), |
| LIGHT | transcript variant 1, mRNA, nucleic acid sequence |
| (TNFSF14) | CGAGACTCCATCTCAAAAACAAAACAAATAAACGAACAAAAAAACCCACAACG |
| Bold: amino | TATTATTTTCTTGTTTACGAGGTTTCTTGTCTCTCTGGCTCCACCAGAAGAGGAG |
| acids 92-240 | CAGGGACCCTTCTTGCTGTTGTTCATTGCTGCATCCCCCACACCGAGAGCAGAG |
| | CCTGGCATGGGCAGAAAGTCCTCAGTCGATATTTGGTGGCCCCAAGCGAATGA |
| | AGCATCCAAGAAGGGAAAGCTGGGGGCTCCCCACTGCACTTGCCACCTGAGTC |
| | ACATTTTCAGAAGCCTCTGGAAAGTCGTGCACAGCCCAGGAGTGTTGAGCAATT |
| | TCGGTTTCCTCTGAGGTTGAAGGACCCAGGCGTGTCAGCCCTGCTCCAGACACC |
| | TTGGGCATGGAGGAGAGTGTCGTACGGCCCTCAGTGTTTGTGGTGGATGGACAG |
| | ACCGACATCCCATTCACGAGGCTGGGACGAAGCCACCGGAGACAGTCGTGCAG |
| | TGTGGCCCGGGTGGGTCTGGGTCTCTTGCTGTTGCTGATGGGGGCCGGGCTGGC |
| | CGTCCAAGGCTGGTTCCTCCTGCAGCTGCACTGGCGTCTAGGAGAGATGGTCAC |
| | CCGCCTGCCTGACGGACCTGCAGGCTCCTGGGAGCAGCTGATACAAGAGCGAA |
| | GGTCTCACGAGGTCAACCCAGCAGCGCATCTCACAGGGGCCAACTCCAGCT |
| | TGACCGGCAGCGGGGGGCCGCTGTTATGGGAGACTCAGCTGGGCCTGGCC |
| | TTCCTGAGGGGCCTCAGCTACCACGATGGGGCCCTTGTGGTCACCAAAGC |
| | TGGCTACTACTACATCTACTCCAAGGTGCAGCTGGGCGGTGTGGGCTGCC |
| | CGCTGGGCCTGGCCAGCACCATCACCCACGGCCTCTACAAGCGCACACCC |
| | CGCTACCCCGAGGAGCTGGAGCTGTTGGTCAGCCAGCAGTCACCCTGCGG |
| | ACGGGCCACCAGCAGCTCCCGGGTCTGGTGGGACAGCAGCTTCCTGGGTG |
| | GTGTGGTACACCTGGAGGCTGGGGAGAAGGTGGTCGTCCGTGTGCTGGAT |
| | GAACGCCTGGTTCGACTGCGTGATGGTACCCGGTCTTACTTCGGGGCTTTC |
| | ATGGTGTGAAGGAAGGAGCGTGGTGCATTGGACATGGGTCTGACACGTGGAGA |
| | ACTCAGAGGGTGCCTCAGGGGAAAGAAAACTCACGAAGCAGAGGCTGGGCGTG |
| | GTGGCTCTCGCCTGTAATCCCAGCACTTTGGGAGGCCAAGGCAGGCGGATCACC |
| | TGAGGTCAGGAGTTCGAGACCAGCCTGGCTAACATGGCAAAACCCCATCTCTAC |
| | TAAAAATACAAAAATTAGCCGGACGTGGTGGTGCCTGCCTGTAATCCAGCTACT |
| | CAGGAGGCTGAGGCAGGATAATTTTGCTTAAACCCGGGAGGCGGAGGTTGCAG |
| | TGAGCCGAGATCACACCACTGCACTCCAACCTGGGAAACGCAGTGAGACTGTG |
| | CCTCAAAAAAAAGAAAGGAAGAAAAAAGAAAACTCAGGAAACAGATCTTGGG |
| | GGACACTCCAGGGAACCCAAAACTCAAAGGCGGAGAGCTCAGTGGGCACCACC |
| | AAGGCGAGATGAAGCCCCAGCAGGCACCTTCAGAAGACCCACGTAGACTGCAG |
| | ACCCTGCCACGGACAATACTAAGGACAAAAACCCAGAGACTTGGGGTCTGTGG |
| | GCCCCCAAACATGGGGTAAAGTTGATTTGCCTGATATTCAGGAAGAAGGGGTG |
| | AGGGGTGGGTATTTATGCTTTTGATTCAGAAGAAAGTGGGGCTTGGGATTCCAG |
| | GGACTTGGCTGGGGGTGGGAAACTTCATCCACTTCCCTACTCTCATCATGAGTA |
| | CGGACAGGGTGGGCGGGAGACTGATCATCGGGACTCATCATGAAGAGCCCAGC |
| | CCCACCCCACATACTCAGATCCCACCCACAGACTGGTGGCCACACCTCAGCCTG |
| | GTCACAAAGAGTTACACTCAGATACATGAGCACGGCAGCGTGCTCATAACTGTT |
| | TAACAACCAGCTGTCCTGGGAGGGGGACAGCTTTGTAATGTTTGCCAATTTCCA |
| | TGGTGTAAATGCTACCACCATGGCTGATTTCATCACTGCCAAGCATAGACATCC |
| | CTAATAGGACACCACGGATCTGTCCCCGGCATCCGGCCCAGGGCCTGGCACAA |
| | AGCATGCTCTAGGGAAATGCTTGCTGATTGAAAGGAAGGAAGAATGACTCTAC |
| | AGTCACACCTATGGCATCCCACAAAATCTGTCACATGGCTGCATAATCTCAGCC |
| | ACTCTTTCACAACTATAGACTCATACACGCGAAGTGCCAGATTCATGCACAACC |
| | ACACAATCACATGGAAGTCACAGACGGCATCACAGACAGTCACAGCACTGTGT |
| | GTATGTTATAACACAAGCACACAAAACTCAGACAGCATCCCAGCTACACAGCC |
| | ACTCCCAGAGGTGTCACCGTCACACTTGGTAATTAATACTCATTACATTAGACA |
| | CAGACAGACCAAGTTATAGTCAGACCTGGTTACACACATACACACACACAATA |
| | TCACCATGACAAATACACATTACACACACACAACATCACAATGACAAACACAC |
| | ATTACACACACAACATCACGATGACAAACACACATTACACACACAACATCACG |
| | ATGACAAACACACATTACACACACATCACAATGACAAACACAACATTACACAC |
| | ACACAACATCACAATGACACACACATCACACACACATCACAATGACAAACACA |
| | CAACATTACACACATATACACACAGCCTGAGGGCCCTCCCCAGCCCAGACTAAC |
| | ACATCTCGGGGTGAGGACCAGACCTTGTTCATAACCCTGGGCCTCTTAACCACT |
| | GATCTTTGAAATAAATGGCAAATAGTTGTACCTGGATCTGTCTAGTTCTTAGGG |
| | GAACAAACTGAAGAAGGGTGGAGAGGAATTGTCAGGCCTAAAGAGCCCCACA |
| | GGGAAAGGGAGGAGTCGGATGGGGGGCAACCATCAGCAACAAGTGGTGGCTC |
| | CTAGAGGCAGAGGGATGGAGGTAATGACCCATGGAGGTCATTCTACAGATGAG |
| | GAACCTGGACCCAGTTGGCTCAAGTCCATGCAGGAAATGTGGGGGAAACCAGA |
| | GACCTCACGTCTGGATCTGGCTTCCTCTCCAATCCACAATTCCTGAGGAAGTAG |
| | AGGCTACATCCCGCAAGACGCCCTTATTAGACACATCCAGGACAGAATGACAA |
| | TCCGCCAAGCCAGCTGGAAGCATAAAACACAGGGAGCTGGTGGGTTGGGTGGG |
| | GGCAGATAATGATATGCATACAAATTAGAGGGTCTATGCAAATGAGCATTGCT |
| | GCAGTGTGGCTGGAGGGAATCCTTAGTTCCTAGGATTCTAGGATATGGGTTTCG |
| | ACCCCAGAGGTGAATGTATTGTTATTATTGTTTTGTTGTTGTTGTGAATGACAAG |
| | TCAAAATTTGTGGGTTATTGTTGTTATCGCCAATAGTATTCTTGTCATTGTTGCA |
| | CAGTACAGAGATGAAGGAAACAGATTTTGCAATCAGATGATCCTGGGTTCTGA |
| | GTCCACTCTGCCACTCACCAGCTATATGACCTCCAGCAATTTCCATCACCTCTCA |
| | ATGCTTCAGTTTCCCCATCGGCAAGATGGTTGTGGGGGGAGAGGAACAACAGT |
| | ACAGATTCACCATCCCAAATTCAAAATGCTCCAAAATCTAGGCCGGGCGTGGTG |
| | GCTCATACCTGTAATCCCAGCACTTTGGGAGGTCAAAGTGGACGGATAACCTGA |
| | GGTCAGGAGCTCCAGACCAGCCTGGCCAACATGGCGAAACCCCATCTCTACTA |
| | AAAATACAAAAAATTACCTGGGTGTGGTGGGGGGCACCTGTAACCCCAGCTAC |
| | TCGGGAGGCTGAGGCAGGAACCCTGGAGGTTGAGGTTGCAGTGAGCTGAGATC |
| | ACACCACTGCACTCCAGCCTGGGTGACAGAGCAAGGCTCCCATCTCAAAAAAC |
| | AAAAAAACATGCTCCAAAATCTGAAACTCTTTGAGCCCCAGTGTGATGCCACAA |
| | GTGGGAAATTCCACAACTCATCACATGTGATAGATTGCAGTGGAAATGCAGGC |
| | ACACACCACGAAGTTTACTCAGCATCCTCAAAGGAAATCCCCGTCAGTAGCTAT |
| | ATATCATTTTCTCACATGCCAGATAGGTATCTCTCATCTTTTACTGTTAGGTACT |
| | TCTGTGTTGAATAGGTGGAGGAAAATGATTGCTGGTTAGTAGTATATAAATTCA |
| | GAGTCAGGAAGGATGGTGATGTCGGCTGGGTGCAGTGGCTCATGCCTGTAATTC |
| | CAATGTGATACCCTACCTTGTGTTTAACGTGATTGACTCTCCCTTAGCTGAGAGG |
| | GCCAGGCAGACTCTATTTTGGCTTCTTCGCTTGCAGTCTCTCACCCACCCCCCTT |
| | CCTCAAGGACTTAAGCTGACTCCCAGCACATCCAAGAATGCGATTACTGATAAG |
| | ATACTGTGACAAGCTATATCCACAATTCCCAGGAATTCGTCCGGTTGATAGCAC |
| | CCAAAGCCCCCGCGTCTATCACCTTGTGATAGATTTAAAGCCCCTGCACCTGGA |
| | ACTGTTTGTTTTTCTGTTACCATTTATCTTTTTCACTTTCTTGCCTGTTTTGCTTCT |
| | GTAAAATTGCTTCAGCTCGGCTCCCTCTTCCCCTTCTAAACCAAGGTATAAAAA |
| | GAAACCTAGCCCCTTCTTTGGGGTGGAGAGAATTTTGAGCGCTAGCCGTCTCTC |
| | AGTCGCCGGCTAATAAAGGACTCCTGAATTAGTCTAA (SEQ ID NO: 117) |
| | >NP 003798.2 tumor necrosis factor ligand superfamily |
| | member 14 isoform 1 [Homo sapiens], nucleic amino acid sequence |
| | MEESVVRPSVFVVDGQTDIPFTRLGRSHRRQSCSVARVGLGLLLLLMGAGLAVQG |
| | WFLLQLHWRLGEMVTRLPDGPAGSWEQLIQERRSHEVNPAAHLTGANSSLTGSG |
| | GPLLWETQLGLAFLRGLSYHDGALVVTKAGYYYIYSKVQLGGVGCPLGLAST |
| | ITHGLYKRTPRYPEELELLVSQQSPCGRATSSSRVWWDSSFLGGVVHLEAGEK |
| | VVVRVLDERLVRLRDGTRSYFGAFMV (SEQ ID NO: 118) |
| Human | >NM_003810.4 Homo sapiens TNF superfamily member 10 (TNFSF10), |
| TRAIL | transcript variant 1, mRNA, nucleic acid sequence |
| (TNFSF10) | GACCGGCTGCCTGGCTGACTTACAGCAGTCAGACTCTGACAGGATCATGGCTAT |
| Bold: amino | GATGGAGGTCCAGGGGGGACCCAGCCTGGGACAGACCTGCGTGCTGATCGTGA |
| acids 120-281 | TCTTCACAGTGCTCCTGCAGTCTCTCTGTGTGGCTGTAACTTACGTGTACTTTAC |
| | CAACGAGCTGAAGCAGATGCAGGACAAGTACTCCAAAAGTGGCATTGCTTGTT |
| | TCTTAAAAGAAGATGACAGTTATTGGGACCCCAATGACGAAGAGAGTATGAAC |
| | AGCCCCTGCTGGCAAGTCAAGTGGCAACTCCGTCAGCTCGTTAGAAAGATGATT |
| | TTGAGAACCTCTGAGGAAACCATTTCTACAGTTCAAGAAAAGCAACAAAATATT |
| | TCTCCCCTAGTGAGAGAAAGAGGTCCTCAGAGAGTAGCAGCTCACATAACTGG |
| | GACCAGAGGAAGAAGCAACACATTGTCTTCTCCAAACTCCAAGAATGAAAAGG |
| | CTCTGGGCCGCAAAATAAACTCCTGGGAATCATCAAGGAGTGGGCATTCATTCC |
| | TGAGCAACTTGCACTTGAGGAATGGTGAACTGGTCATCCATGAAAAAGGGTTTT |
| | ACTACATCTATTCCCAAACATACTTTCGATTTCAGGAGGAAATAAAAGAAAACA |
| | CAAAGAACGACAAACAAATGGTCCAATATATTTACAAATACACAAGTTATCCT |
| | GACCCTATATTGTTGATGAAAAGTGCTAGAAATAGTTGTTGGTCTAAAGATGCA |
| | GAATATGGACTCTATTCCATCTATCAAGGGGGAATATTTGAGCTTAAGGAAAAT |
| | GACAGAATTTTTGTTTCTGTAACAAATGAGCACTTGATAGACATGGACCATGAA |
| | GCCAGTTTTTTTGGGGCCTTTTTAGTTGGCTAACTGACCTGGAAAGAAAAAGCA |
| | ATAACCTCAAAGTGACTATTCAGTTTTCAGGATGATACACTATGAAGATGTTTC |
| | AAAAAATCTGACCAAAACAAACAAACAGAAAACAGAAAACAAAAAAACCTCT |
| | ATGCAATCTGAGTAGAGCAGCCACAACCAAAAAATTCTACAACACACACTGTT |
| | CTGAAAGTGACTCACTTATCCCAAGAGAATGAAATTGCTGAAAGATCTTTCAGG |
| | ACTCTACCTCATATCAGTTTGCTAGCAGAAATCTAGAAGACTGTCAGCTTCCAA |
| | ACATTAATGCAATGGTTAACATCTTCTGTCTTTATAATCTACTCCTTGTAAAGAC |
| | TGTAGAAGAAAGAGCAACAATCCATCTCTCAAGTAGTGTATCACAGTAGTAGC |
| | CTCCAGGTTTCCTTAAGGGACAACATCCTTAAGTCAAAAGAGAGAAGAGGCAC |
| | CACTAAAAGATCGCAGTTTGCCTGGTGCAGTGGCTCACACCTGTAATCCCAACA |
| | TTTTGGGAACCCAAGGTGGGTAGATCACGAGATCAAGAGATCAAGACCATAGT |
| | GACCAACATAGTGAAACCCCATCTCTACTGAAAGTACAAAAATTAGCTGGGTGT |
| | GTTGGCACATGCCTGTAGTCCCAGCTACTTGAGAGGCTGAGGCAAGAGAATTGT |
| | TTGAACCCGGGAGGCAGAGGTTGCAGTGTGGTGAGATCATGCCACTACACTCC |
| | AGCCTGGCGACAGAGCGAGACTTGGTTTCAAAAAAAAAAAAAAAAAAAACTTC |
| | AGTAAGTACGTGTTATTTTTTTCAATAAAATTCTATTACAGTATGTCATGTTTGC |
| | TGTAGTGCTCATATTTATTGTTGTTTTTGTTTTAGTACTCACTTGTTTCATAATAT |
| | CAAGATTACTAAAAATGGGGGAAAAGACTTCTAATCTTTTTTTCATAATATCTTT |
| | GACACATATTACAGAAGAAATAAATTTCTTACTTTTAATTTAATATGA (SEQ ID |
| | NO: 119) |
| | >NP_003801.1 tumor necrosis factor ligand superfamily |
| | member 10 isoform 1 [Homo sapiens], amino acid sequence |
| | MAMMEVQGGPSLGQTCVLIVIFTVLLQSLCVAVTYVYFTNELKQMQDKYSKSGIA |
| | CFLKEDDSYWDPNDEESMNSPCWQVKWQLRQLVRKMILRTSEETISTVQEKQQNI |
| | SPLVRERGPQRVAAHITGTRGRSNTLSSPNSKNEKALGRKINSWESSRSGHSFLS |
| | NLHLRNGELVIHEKGFYYIYSQTYFRFQEEIKENTKNDKQMVQYIYKYTSYPD |
| | PILLMKSARNSCWSKDAEYGLYSIYQGGIFELKENDRIFVSVTNEHLIDMDHEA |
| | SFFGAFLVG (SEQ ID NO: 120) |
| Human | >NM_003701.4 Homo sapiens TNF superfamily member 11 (TNFSF11), |
| RANKL | transcript variant 1, mRNA, nucleic amino acid sequence |
| Bold: amino | GCCCGCTCGCCCGCGCGCCCCAGGACCCAAAGCCGGGCTCCAAGTCGGCGCCC |
| acids 161-317 | CACGTCGAGGCTCCGCCGCAGCCTCCGGAGTTGGCCGCAGACAAGAAGGGGAG |
| | GGAGCGGGAGAGGGAGGAGAGCTCCGAAGCGAGAGGGCCGAGCGCCATGCGC |
| | CGCGCCAGCAGAGACTACACCAAGTACCTGCGTGGCTCGGAGGAGATGGGCGG |
| | CGGCCCCGGAGCCCCGCACGAGGGCCCCCTGCACGCCCCGCCGCCGCCTGCGC |
| | CGCACCAGCCCCCTGCCGCCTCCCGCTCCATGTTCGTGGCCCTCCTGGGGCTGG |
| | GGCTGGGCCAGGTTGTCTGCAGCGTCGCCCTGTTCTTCTATTTCAGAGCGCAGA |
| | TGGATCCTAATAGAATATCAGAAGATGGCACTCACTGCATTTATAGAATTTTGA |
| | GACTCCATGAAAATGCAGATTTTCAAGACACAACTCTGGAGAGTCAAGATACA |
| | AAATTAATACCTGATTCATGTAGGAGAATTAAACAGGCCTTTCAAGGAGCTGTG |
| | CAAAAGGAATTACAACATATCGTTGGATCACAGCACATCAGAGCAGAGAAAGC |
| | GATGGTGGATGGCTCATGGTTAGATCTGGCCAAGAGGAGCAAGCTTGAAGCTC |
| | AGCCTTTTGCTCATCTCACTATTAATGCCACCGACATCCCATCTGGTTCCCA |
| | TAAAGTGAGTCTGTCCTCTTGGTACCATGATCGGGGTTGGGCCAAGATCTC |
| | CAACATGACTTTTAGCAATGGAAAACTAATAGTTAATCAGGATGGCTTTTA |
| | TTACCTGTATGCCAACATTTGCTTTCGACATCATGAAACTTCAGGAGACCT |
| | AGCTACAGAGTATCTTCAACTAATGGTGTACGTCACTAAAACCAGCATCAA |
| | AATCCCAAGTTCTCATACCCTGATGAAAGGAGGAAGCACCAAGTATTGGTC |
| | AGGGAATTCTGAATTCCATTTTTATTCCATAAACGTTGGTGGATTTTTTAAG |
| | TTACGGTCTGGAGAGGAAATCAGCATCGAGGTCTCCAACCCCTCCTTACTG |
| | GATCCGGATCAGGATGCAACATACTTTGGGGCTTTTAAAGTTCGAGATATA |
| | GATTGAGCCCCAGTTTTTGGAGTGTTATGTATTTCCTGGATGTTTGGAAACATTT |
| | TTTAAAACAAGCCAAGAAAGATGTATATAGGTGTGTGAGACTACTAAGAGGCA |
| | TGGCCCCAACGGTACACGACTCAGTATCCATGCTCTTGACCTTGTAGAGAACAC |
| | GCGTATTTACAGCCAGTGGGAGATGTTAGACTCATGGTGTGTTACACAATGGTT |
| | TTTAAATTTTGTAATGAATTCCTAGAATTAAACCAGATTGGAGCAATTACGGGG |
| | TGACCTTATGAGAAACTGCATGTGGGCTATGGGAGGGGTTGGTCCCTGGTCATG |
| | TGCCCCTTCGCAGCTGAAGTGGAGAGGGTGTCATCTAGCGCAATTGAAGGATCA |
| | TCTGAAGGGGCAAATTCTTTTGAATTGTTACATCATGCTGGAACCTGCAAAAAA |
| | TACTTTTTCTAATGAGGAGAGAAAATATATGTATTTTTATATAATATCTAAAGTT |
| | ATATTTCAGATGTAATGTTTTCTTTGCAAAGTATTGTAAATTATATTTGTGCTAT |
| | AGTATTTGATTCAAAATATTTAAAAATGTCTTGCTGTTGACATATTTAATGTTTT |
| | AAATGTACAGACATATTTAACTGGTGCACTTTGTAAATTCCCTGGGGAAAACTT |
| | GCAGCTAAGGAGGGGAAAAAAATGTTGTTTCCTAATATCAAATGCAGTATATTT |
| | CTTCGTTCTTTTTAAGTTAATAGATTTTTTCAGACTTGTCAAGCCTGTGCAAAAA |
| | AATTAAAATGGATGCCTTGAATAATAAGCAGGATGTTGGCCACCAGGTGCCTTT |
| | CAAATTTAGAAACTAATTGACTTTAGAAAGCTGACATTGCCAAAAAGGATACAT |
| | AATGGGCCACTGAAATCTGTCAAGAGTAGTTATATAATTGTTGAACAGGTGTTT |
| | TTCCACAAGTGCCGCAAATTGTACCTTTTTTGTTTTTTCAAAATAGAAAAGTTAT |
| | TAGTGGTTTATCAGCAAAAAAGTCCAATTTTAATTTAGTAAATGTTATCTTATAC |
| | TGTACAATAAAAACATTGCCTTTGAATGTTAATTTTTTGGTACAAAAATAAATTT |
| | ATATGAAAACCTGC (SEQ ID NO: 121) |
| | >NP_003692.1 tumor necrosis factor ligand superfamily |
| | member 11 isoform 1 [Homo sapiens], amino acid sequence |
| | MRRASRDYTKYLRGSEEMGGGPGAPHEGPLHAPPPPAPHQPPAASRSMFVALLGL |
| | GLGQVVCSVALFFYFRAQMDPNRISEDGTHCIYRILRLHENADFQDTTLESQDTKLI |
| | PDSCRRIKQAFQGAVQKELQHIVGSQHIRAEKAMVDGSWLDLAKRSKLEAQPFAH |
| | LTINATDIPSGSHKVSLSSWYHDRGWAKISNMTFSNGKLIVNQDGFYYLYANIC |
| | FRHHETSGDLATEYLQLMVYVTKTSIKIPSSHTLMKGGSTKYWSGNSEFHFYSI |
| | NVGGFFKLRSGEEISIEVSNPSLLDPDQDATYFGAFKVRDID (SEQ ID NO: 122) |
| Human | >NM_005118.4 Homo sapiens TNF superfamily member 15 (TNFSF15), |
| TL1A | transcript variant 1, mRNA, nucleic acid sequence |
| Bold: amino | AGAGGTGCCTCCAGGAGCAGCAGGAGCATGGCCGAGGATCTGGGACTGAGCTT |
| acids 93-251 | TGGGGAAACAGCCAGTGTGGAAATGCTGCCAGAGCACGGCAGCTGCAGGCCCA |
| | AGGCCAGGAGCAGCAGCGCACGCTGGGCTCTCACCTGCTGCCTGGTGTTGCTCC |
| | CCTTCCTTGCAGGACTCACCACATACCTGCTTGTCAGCCAGCTCCGGGCCCAGG |
| | GAGAGGCCTGTGTGCAGTTCCAGGCTCTAAAAGGACAGGAGTTTGCACCTTCAC |
| | ATCAGCAAGTTTATGCACCTCTTAGAGCAGACGGAGATAAGCCAAGGGCACA |
| | CCTGACAGTTGTGAGACAAACTCCCACACAGCACTTTAAAAATCAGTTCCC |
| | AGCTCTGCACTGGGAACATGAACTAGGCCTGGCCTTCACCAAGAACCGAAT |
| | GAACTATACCAACAAATTCCTGCTGATCCCAGAGTCGGGAGACTACTTCAT |
| | TTACTCCCAGGTCACATTCCGTGGGATGACCTCTGAGTGCAGTGAAATCAG |
| | ACAAGCAGGCCGACCAAACAAGCCAGACTCCATCACTGTGGTCATCACCAA |
| | GGTAACAGACAGCTACCCTGAGCCAACCCAGCTCCTCATGGGGACCAAGT |
| | CTGTATGCGAAGTAGGTAGCAACTGGTTCCAGCCCATCTACCTCGGAGCCA |
| | TGTTCTCCTTGCAAGAAGGGGACAAGCTAATGGTGAACGTCAGTGACATCT |
| | CTTTGGTGGATTACACAAAAGAAGATAAAACCTTCTTTGGAGCCTTCTTAC |
| | TATAGGAGGAGAGCAAATATCATTATATGAAAGTCCTCTGCCACCGAGTTCCTA |
| | ATTTTCTTTGTTCAAATGTAATTATAACCAGGGGTTTTCTTGGGGCCGGGAGTAG |
| | GGGGCATTCCACAGGGACAACGGTTTAGCTATGAAATTTGGGGCCCAAAATTTC |
| | ACACTTCATGTGCCTTACTGATGAGAGTACTAACTGGAAAAAGGCTGAAGAGA |
| | GCAAATATATTATTAAGATGGGTTGGAGGATTGGCGAGTTTCTAAATATTAAGA |
| | CACTGATCACTAAATGAATGGATGATCTACTCGGGTCAGGATTGAAAGAGAAA |
| | TATTTCAACACCTTCCTGCTATACAATGGTCACCAGTGGTCCAGTTATTGTTCAA |
| | TTTGATCATAAATTTGCTTCAATTCAGGAGCTTTGAAGGAAGTCCAAGGAAAGC |
| | TCTAGAAAACAGTATAAACTTTCAGAGGCAAAATCCTTCACCAATTTTTCCACA |
| | TACTTTCATGCCTTGCCTAAAAAAAATGAAAAGAGAGTTGGTATGTCTCATGAA |
| | TGTTCACACAGAAGGAGTTGGTTTTCATGTCATCTACAGCATATGAGAAAAGCT |
| | ACCTTTCTTTTGATTATGTACACAGATATCTAAATAAGGAAGTATGAGTTTCAC |
| | ATGTATATCAAAAATACAACAGTTGCTTGTATTCAGTAGAGTTTTCTTGCCCACC |
| | TATTTTGTGCTGGGTTCTACCTTAACCCAGAAGACACTATGAAAAACAAGACAG |
| | ACTCCACTCAAAATTTATATGAACACCACTAGATACTTCCTGATCAAACATCAG |
| | TCAACATACTCTAAAGAATAACTCCAAGTCTTGGCCAGGCGCAGTGGCTCACAC |
| | CTGTAATCCCAACACTTTGGGAGGCCAAGGTGGGTGGATCATCTAAGGCCGGG |
| | AGTTCAAGACCAGCCTGACCAACGTGGAGAAACCCCATCTCTACTAAAAATAC |
| | AAAATTAGCCGGGCGTGGTAGCGCATGGCTGTAATCCTGGCTACTCAGGAGGC |
| | CGAGGCAGAAGAATTGCTTGAACTGGGGAGGCAGAGGTTGCGGTGAGCCCAGA |
| | TCGCGCCATTGCACTCCAGCCTGGGTAACAAGAGCAAAACTCTGTCCAAAAAA |
| | AAAAAAATAAAATAATAACTCCAAGCCTTTAAAAAATATCATCTGAAACTGTTA |
| | CATCAGATTTCTGGCACTCTACTGACTGTGGAAGATAGCCAGCTGACTGGAAGA |
| | TAGCCAGCTGATTAGTTCCCTGAAGAAACCTGAAGACAGATACCTGGTTAACTA |
| | GATCAACTACACTGCCAACTTGTTTGATGCTGAGAGACAATGGACTTATTCCAT |
| | GGGGGAAGGGAAAAAAGAAGTCAATCACCAAATCTGAAGAAGTTAACCTAGAT |
| | CTTTGAGGTTTGATTTGCAACTTTATATGCAGAGTATTATGTGGGTATTTTCCCT |
| | TAAAATATTCAAAGGGATTTACATATGGGATTAGCTAATGAGCCTAGCCAAGAC |
| | CTTCCCTGGAGGACAGGCTGGTCATTGCGGAGGTCCCTTCTGTGCTTCAGTGGG |
| | TTCATATCCTCTAGTCCGTATGATTTTCCTACGCTAATATGTCAAGGGCAGGAG |
| | AGGCAGCTCTGTTCTCCTAGCCTTTGTTGACTTGTCTGCAAAGCAGGAATCTGCC |
| | CATTTGTTTCCAAGGAGCAAATGAGCTCATGAGAATGAAAGATGTTAACTTCAT |
| | GCATTCTGTGCCATCTGAGCATTTCGGTATTATATGACTGGTGACCCTTGGCCCG |
| | TATTATAAATGCTTCCTATCCTGGGAGACCTCATGGATGAGTCTGAGAGGAAAT |
| | TTGGCACCAAAATCACTCTCACTCTGGTTTCCAGTAGACTATAGAGGCAGAGAG |
| | GCATTTGAGAGGCTCCTGAGCAAAGTGTCCAGTGTAGCAGGAGCACTTCATTAA |
| | TATTTATTGAGTTATAATTAAATAAAAATTAATTTCTGATTTCTCAGTTTGGAGG |
| | TTAAGGCTCTAAATATATTTTCTAACCTCTGCTAGGCTAACTTAAGCCAGGCCTT |
| | TTTCTTGCCTTCCCTTTCTCAAAACAGTCAGCACAGACTCAGTGGGAGCACAGA |
| | GGAGTGTGGTCACCTCCACCTGGCTCACCAGAGTCTTCATAGAGGAAGTGAAGC |
| | CTGGAAGAAACTGGGCGGGCCCCAGATGACCACAGGGAAAGGGCATCTCAGAT |
| | GGAGGAATTACCCTTGACTTAAAGCAGAAAAGAAAGATTTCTCAGTAACTCCA |
| | AAACTTGCTTGATAGGAGAATATTCCCTCAACCAATTCCTAGGACAATATTTAT |
| | TGGTAGATCAAGAATGTTTCCTCAATAACTCTAGTCTAGCTCCATGATCAGAAC |
| | TAACACCCATTAAAAACATAAAATGTTCTTTCTGAACCGGTCTTCATGGTGCGT |
| | GAGAGCACCAAGCAGCTTTGGTATGCAGGAGGAGTTTTGCACAGAAGAGTGGC |
| | CTGCTCAAACCTGCCCACTGTTCTGTAGGTGATCTGGTGGATCTGGAAATTTATC |
| | CCAAGACAGGAATTTCCTAATATTCGAAGACATTTGAGGCTTTGGGAAATTCTC |
| | TGCTGTGCATTTATTTGGCTCCTGTCATAAGCTTGTTTTTTAAAGAATGTATCAT |
| | AGCTCAAGTTTTTACTGCTGATTTTGTTAAATTCTGTATAGTATATTTTTTACGG |
| | AAAGGCACAGTCAGACATTCCTAATAGGGCTCATGTCAGAACTTCTGTTCCCAA |
| | GGCATTATCTCCATAGCAAAAATTAGTGCACTGTTTTCAAAAGTGAGGTGGGAA |
| | AATGCTTTTAAGATCATGTGATGTTCCCCTAAAAGGGGTTAATGGGGTGTATTC |
| | AGGGTTTGGGAGGGAGGAAGAAGCATGCTTTAGAAAACAGTAAATTTAGGGAG |
| | AAAATGCTTTGTTGGTTAAATGTCACTCAAAAGGCTGAATTCAAATCAATTCCA |
| | CAAACATTTACTGAGTACCTACTGCCCCTGGGGACACAGAGATAAATTATTTAG |
| | TCTCAGACACACTCATTCTAACTTCCCAGCACCTCTACTGTCTGCAGATTCTTTA |
| | ATTTATTTTGGTTGTATTAGCTAATTAATTCGTAAACTTTAGGCACATGGATCTA |
| | TTCTCATTATGAAAATGGATGCCATTTGATTAAGGCTGATGACTAACAAAATGA |
| | TTTGTGTTTACTCGAAGTGTTTTTTTAAAAATAGCTACTCAAGGATAGTTTTCCA |
| | TAAATCAAGAAGGTAAAAAAGTTCCCATTTTTTATTGTAGAATCCATTATTTAA |
| | ACTACATGTAGAGACAGGTTATTATTTGCTATATTCAAGTTTGGTCATCAATACC |
| | CTTAAAAATATTAGAATTTTATGGATGACCCAGAAATGCTTTGAAAATCTGTGT |
| | TCCTCAGCAAATACAGAGACCATGATCAAAATGCACAGAATCACTAACATTTTG |
| | ATGCTAGCATGGTTTCAGTCTATTTGGCAGAACAGAATTGATTATGCTACTAAA |
| | ATTTCTTTTTCTTTTTTTTTTTTTTTTTTTTTGAGACAGAGTCTTGCTTTGTCACCC |
| | AGGCTGAAGTGCAGTGGCAGGATCTCAGTTCACTGCAACCTCTGCCTCCCAGGT |
| | TCACGCCATTCTCCTGCTTCAGCCTCCCGAGTAGCTGGGACTACAGGCTCCCAC |
| | CACCATGCCCGGCTAATTTTTTGCATTTTTAGTAGAGACGGGGTTTCACCGTGTT |
| | AGCCAGGATGGTCTCGATCTCCTGACCTCGTGATCCGCCCGCCTCAGCCTTCCA |
| | AAGTGCTGGGATTACAGGCGTGAGCCACTGTGCCCGGACTCTGATTTTTTTTTTA |
| | CTAAGGTACAGTAAGAAAAGGGAAAAGTGTACGTTTTCACTTCCTGAAATATGT |
| | CAGGTTGAATCAATAATAGAGCACACCAGAACTCTTGGCTCCATTTCAACCTAA |
| | ACTATTCAGTTCTCATCACCCCAGAGGAAATTCCGCCTCTGTGCTGGTCAGTAA |
| | TCCCCCTGGATTATAAAAGTTTAACTAACTCACTGTGCACAAGGCACGGCCATT |
| | GCCAACATTCTCTTGCAAGGTATTTTCCCAAGCCCTTACCCAATTCTGTTTCCAT |
| | GATTGTGACATTGGGGATTAATTCTGCAAGACAGAACTGTTTATATTCTGTACC |
| | TTAAAAACACATGCAAACATCTCTTGCCTTAAGATTTCTGGCTTTCCTATGGCCC |
| | AGAGTCCTAGAAGTGTTTTGATATTTGTAGCAGAATTTTCAAGTGTACATCCTTA |
| | TCCTGGATATTAACATTTTTGCATCATATTGGCAGCTGGACCTACAGAGAATTT |
| | AGTAGACTGTTAACCTAATAAGCCTTGAATCCTTTTGCACCAGTGGTGAGAGAA |
| | TGTGGATCAGAGCCATCACCTCCATGCCCCGTCACCCTCTAACAACCACATTTA |
| | CAACTTCCCCAGCTCTGAGACACACTTGCCTCCACCCCTTCCATCACCCCATTTT |
| | AAGATGAAAATACCACACCAGCCTGGAAGGAAGAAGTTACTTGCCCAGGGCCA |
| | CATAGTGAGTTAAGGGCTGATCTAGAGCTAGGAAGCTGTCTTCCTGAACCATAA |
| | TCCTGGACTCTTCTAACCTCTCTACTCATCGCAAATAGAGTTCATTTTAGTGATT |
| | TGAAGGAAGATGGGACAAGTATTTTCAAACACCTGTAGGACAACATGGAAGTG |
| | GGAGGAGACTTCTACTGTAGCTCCCCAGAGAAGAGAGCTAGGGCTACAGAGTT |
| | GCAGTTACAAGGTTGCCCTCTCTGGCTTGATCCCCAAAGGAATTTTCTACTCCA |
| | AAATAGAATTTTTCTAGGATGCTATTTCTCAGTCCCTGGAGATACTCAAACAAA |
| | GGGCTTGTCACAAGGGTTTTTGTAGAAGCTATTCTTCACAGAGGTTGGGGGAGA |
| | GATTAAGCCAAAGGATCTCTGAGGTCTTTTTCAAATCTATAATTATGTGGCCTTT |
| | TGTTCATTGACTTCCATGTGTTCTAGTTGATCATTACAAACCTGGCAGGCCTTCT |
| | CAAGGGTTCAGTAATTAGCTGTCATTTCCCATTTGTCCAGAGAGTGTCCAACAC |
| | AAAATACCCCTAAGATCTTGGCCAATAGAGAAATGTCATGGAATTTTAGAAATG |
| | ACAGTATCTGCGGAGTTTATTCCAAGTTATATCATTTCAAAGATGAAGAAACCC |
| | AGGCTCAGAGGGAGCCATCACATCCACACCCTGTCACCCTTCGTGGCCAGTGCC |
| | AGACAGTAGCTAGTTGGATGCTAAAAGTAGAATTTAGATATCTTAACAATAAGC |
| | CCAGCAGTCTTTCAACTTCATTCGTAAATCATTTTTGTTTTGAGCATCTGTCACG |
| | TGGCAGCACTTGCCTGGATACTGGAGAGCTGAGAAGGAATGCGACAGGCAAGT |
| | CCTACTCTCACAGTGTATACATTCAGGAGGAACAAGACACACAGTGCCAAGTA |
| | AATAAAGTAGCTGAACTTCATCAAATGATTTTATTCTTAAAGTCATTAAAGCAT |
| | GTAATGTTCCCCTTTTTTTGTTTCAGGGGTGTACAGATTGAAGAAGTGTAGGTGT |
| | TTATGTGGTTTTAGTGACAAACCCCATGTGCTTTCATTGATTTTATGTTTTATGTT |
| | AAAACATCAACCGCAAGGTAAAATGCATATTGTATGTTGTTGGATACGTACTTA |
| | ACTGGTATGCATCCCATGTCTTTGGGTACTAGTGTATGAATTCTAATCTCTGTAA |
| | ATGAAATGTTGTATGTGTTAATATATTTAATAGATGTAACTTAATAAACTGGCA |
| | TTGAAGACTGAA (SEQ ID NO: 123) |
| | >NP_005109.2 tumor necrosis factor ligand superfamily member 15 |
| | isoform VEGI-251 precursor [Homo sapiens], amino acid sequence |
| | MAEDLGLSFGETASVEMLPEHGSCRPKARSSSARWALTCCLVLLPFLAGLTTYLLV |
| | SQLRAQGEACVQFQALKGQEFAPSHQQVYAPLRADGDKPRAHLTVVRQTPTQHF |
| | KNQFPALHWEHELGLAFTKNRMNYTNKFLLIPESGDYFIYSQVTFRGMTSECS |
| | EIRQAGRPNKPDSITVVITKVTDSYPEPTQLLMGTKSVCEVGSNWFQPIYLGAM |
| | FSLQEGDKLMVNVSDISLVDYTKEDKTFFGAFLL (SEQ ID NO: 124) |
| Human | >NM_000639.3 Homo sapiens Fas ligand (FASLG), transcript |
| FASL | variant 1, mRNA, nucleic acid sequence |
| Bold: amino | AGCAGTCAGCAACAGGGTCCCGTCCTTGACACCTCAGCCTCTACAGGACTGAGA |
| acids 144-281 | AGAAGTAAAACCGTTTGCTGGGGCTGGCCTGACTCACCAGCTGCCATGCAGCA |
| | GCCCTTCAATTACCCATATCCCCAGATCTACTGGGTGGACAGCAGTGCCAGCTC |
| | TCCCTGGGCCCCTCCAGGCACAGTTCTTCCCTGTCCAACCTCTGTGCCCAGAAG |
| | GCCTGGTCAAAGGAGGCCACCACCACCACCGCCACCGCCACCACTACCACCTCC |
| | GCCGCCGCCGCCACCACTGCCTCCACTACCGCTGCCACCCCTGAAGAAGAGAG |
| | GGAACCACAGCACAGGCCTGTGTCTCCTTGTGATGTTTTTCATGGTTCTGGTTGC |
| | CTTGGTAGGATTGGGCCTGGGGATGTTTCAGCTCTTCCACCTACAGAAGGAGCT |
| | GGCAGAACTCCGAGAGTCTACCAGCCAGATGCACACAGCATCATCTTTGGAGA |
| | AGCAAATAGGCCACCCCAGTCCACCCCCTGAAAAAAAGGAGCTGAGGAAAGT |
| | GGCCCATTTAACAGGCAAGTCCAACTCAAGGTCCATGCCTCTGGAATGGGA |
| | AGACACCTATGGAATTGTCCTGCTTTCTGGAGTGAAGTATAAGAAGGGTGG |
| | CCTTGTGATCAATGAAACTGGGCTGTACTTTGTATATTCCAAAGTATACTTC |
| | CGGGGTCAATCTTGCAACAACCTGCCCCTGAGCCACAAGGTCTACATGAG |
| | GAACTCTAAGTATCCCCAGGATCTGGTGATGATGGAGGGGAAGATGATGA |
| | GCTACTGCACTACTGGGCAGATGTGGGCCCGCAGCAGCTACCTGGGGGCA |
| | GTGTTCAATCTTACCAGTGCTGATCATTTATATGTCAACGTATCTGAGCTCT |
| | CTCTGGTCAATTTTGAGGAATCTCAGACGTTTTTCGGCTTATATAAGCTCTA |
| | AGAGAAGCACTTTGGGATTCTTTCCATTATGATTCTTTGTTACAGGCACCGAGA |
| | ATGTTGTATTCAGTGAGGGTCTTCTTACATGCATTTGAGGTCAAGTAAGAAGAC |
| | ATGAACCAAGTGGACCTTGAGACCACAGGGTTCAAAATGTCTGTAGCTCCTCAA |
| | CTCACCTAATGTTTATGAGCCAGACAAATGGAGGAATATGACGGAAGAACATA |
| | GAACTCTGGGCTGCCATGTGAAGAGGGAGAAGCATGAAAAAGCAGCTACCAGG |
| | TGTTCTACACTCATCTTAGTGCCTGAGAGTATTTAGGCAGATTGAAAAGGACAC |
| | CTTTTAACTCACCTCTCAAGGTGGGCCTTGCTACCTCAAGGGGGACTGTCTTTCA |
| | GATACATGGTTGTGACCTGAGGATTTAAGGGATGGAAAAGGAAGACTAGAGGC |
| | TTGCATAATAAGCTAAAGAGGCTGAAAGAGGCCAATGCCCCACTGGCAGCATC |
| | TTCACTTCTAAATGCATATCCTGAGCCATCGGTGAAACTAACAGATAAGCAAGA |
| | GAGATGTTTTGGGGACTCATTTCATTCCTAACACAGCATGTGTATTTCCAGTGCA |
| | ATTGTAGGGGTGTGTGTGTGTGTGTGTGTGTGTGTGTGTGTATGACTAAAGAGA |
| | GAATGTAGATATTGTGAAGTACATATTAGGAAAATATGGGTTGCATTTGGTCAA |
| | GATTTTGAATGCTTCCTGACAATCAACTCTAATAGTGCTTAAAAATCATTGATTG |
| | TCAGCTACTAATGATGTTTTCCTATAATATAATAAATATTTATGTAGATGTGCAT |
| | TTTTGTGAAATGAAAACATGTAATAAAAAGTATATGTTAGGATACAAATAA |
| | (SEQ ID NO: 125) |
| | >NP_000630.1 tumor necrosis factor ligand superfamily |
| | member 6 isoform 1 [Homo sapiens], amino acid sequence |
| | MQQPFNYPYPQIYWVDSSASSPWAPPGTVLPCPTSVPRRPGQRRPPPPPPPPPLPPPP |
| | PPPPLPPLPLPPLKKRGNHSTGLCLLVMFFMVLVALVGLGLGMFQLFHLQKELAEL |
| | RESTSQMHTASSLEKQIGHPSPPPEKKELRKVAHLTGKSNSRSMPLEWEDTYGIV |
| | LLSGVKYKKGGLVINETGLYFVYSKVYFRGQSCNNLPLSHKVYMRNSKYPQD |
| | LVMMEGKMMSYCTTGQMWARSSYLGAVENLTSADHLYVNVSELSLVNFEES |
| | QTFFGLYKL (SEQ ID NO: 126) |
| Human | >NM_006573.5 Homo sapiens TNF superfamily member |
| BAFF | 13b (TNFSF13B), transcript variant 1, |
| Bold: amino | mRNA, nucleic acid sequence |
| acids 140-285 | AGGGGTAGAGATGCAGAAAGGCAGAAAGGAGAAAATTCAGGATAACTCTCCTG |
| | AGGGGTGAGCCAAGCCCTGCCATGTAGTGCACGCAGGACATCAACAAACACAG |
| | ATAACAGGAAATGATCCATTCCCTGTGGTCACTTATTCTAAAGGCCCCAACCTT |
| | CAAAGTTCAAGTAGTGATATGGATGACTCCACAGAAAGGGAGCAGTCACGCCT |
| | TACTTCTTGCCTTAAGAAAAGAGAAGAAATGAAACTGAAGGAGTGTGTTTCCAT |
| | CCTCCCACGGAAGGAAAGCCCCTCTGTCCGATCCTCCAAAGACGGAAAGCTGCT |
| | GGCTGCAACCTTGCTGCTGGCACTGCTGTCTTGCTGCCTCACGGTGGTGTCTTTC |
| | TACCAGGTGGCCGCCCTGCAAGGGGACCTGGCCAGCCTCCGGGCAGAGCTGCA |
| | GGGCCACCACGCGGAGAAGCTGCCAGCAGGAGCAGGAGCCCCCAAGGCCGGC |
| | CTGGAGGAAGCTCCAGCTGTCACCGCGGGACTGAAAATCTTTGAACCACCAGCT |
| | CCAGGAGAAGGCAACTCCAGTCAGAACAGCAGAAATAAGCGTGCCGTTCAGGG |
| | TCCAGAAGAAACAGTCACTCAAGACTGCTTGCAACTGATTGCAGACAGTGA |
| | AACACCAACTATACAAAAAGGATCTTACACATTTGTTCCATGGCTTCTCAG |
| | CTTTAAAAGGGGAAGTGCCCTAGAAGAAAAAGAGAATAAAATATTGGTCAA |
| | AGAAACTGGTTACTTTTTTATATATGGTCAGGTTTTATATACTGATAAGACC |
| | TACGCCATGGGACATCTAATTCAGAGGAAGAAGGTCCATGTCTTTGGGGAT |
| | GAATTGAGTCTGGTGACTTTGTTTCGATGTATTCAAAATATGCCTGAAACA |
| | CTACCCAATAATTCCTGCTATTCAGCTGGCATTGCAAAACTGGAAGAAGGA |
| | GATGAACTCCAACTTGCAATACCAAGAGAAAATGCACAAATATCACTGGAT |
| | GGAGATGTCACATTTTTTGGTGCATTGAAACTGCTGTGACCTACTTACACCA |
| | TGTCTGTAGCTATTTTCCTCCCTTTCTCTGTACCTCTAAGAAGAAAGAATCTAAC |
| | TGAAAATACCAAAAAAAAAAAAAAAAAAAAAAAAAAAAAAAGTAGTTACCAT |
| | TGCCTTTTCTGTGAGCTATTTGTTTTGGTTTGCTGAAACTAGTCCAAAACAGGAA |
| | ATTTAACAGACAGCCACAGCCAAAGAGTGTCATGTGAATTACAAGAAATAGAG |
| | CCCATTTAGGGAAAGATAGAACTAGAAAGGCTTTTCATTATAATTCCATGTTGA |
| | ACAATTGAGTCATAGCTTCTTATCTTGGAGGAAGGACACAATTCAAAGGGGCA |
| | GTAAGGATTTTGTAAAACGTGGCATCCATAATTTACTATGGAGCAAGTGCCCAC |
| | ATCTCTAGGACATTAAGACATTTATGAGAAATCTCAGGATTCATCTTCTGTTTTT |
| | ATGTTAAATGCACTCCCTCCTTTTCAGTTAACATTATAAAAAGTAAAAAATGAA |
| | AATTTTAGAAATCTTGCATTAGACACATGAAAAAATAACTAAAAGTTTAAATTT |
| | AAATATGAAACAATTTTGCTGAAAATAGTATCCATATACTATTTAAGTCTTTTAT |
| | GGTTATTTCAAGTATACAATTTCTATCTGTAATGTAATATATTACCCACACATTT |
| | TTTTCACAGGAGAGAGAGAATATCCTCATTTGTTTATGCTCATGTGTATTTTCTA |
| | TAGTGAATTTCAGAAACTTTTAATATCAGGTAATTTCAATTTATGCCTATAAAGC |
| | ATTGATTGAAAAATAACTAGAATTGTGCATATATAACACATAATCTCCAACAGA |
| | AGTTACTGAATACATTCATACTAATGTAATGTAATTTCCCTTTATTTCTTGCTCTT |
| | CTGTTTCAAACTGCTGCTATTGTAGTTTACATATCCCAACCTTTAAAAATATTCC |
| | TCTTATTAGCTTTATATTCACTTTATAGAAGTTGAGTTTTAATTAAAATTCTTGG |
| | CATCCTGAAGTATGTCACATAGCATGTGCTCCTTATAAATATGTTGATATCTCAG |
| | AAGACAGCATCCCGGTTTTCATTTTATAAAGTACCATACTTAAGAATGCTGTAA |
| | TACTTATCTTTTATAACATGTTTCCTTCGCTTTGCTTGTCTTTTATGTCATCAGTT |
| | TTAACTGTTTACTTCATTTAACAGTTTACATCATTCAACAGTTTACTTCATTAAA |
| | CAGTAGGTGGAAAAATAGATGCCAGTCTATGAAAATCTTCCCATCTATATCAAA |
| | ATACTTTTCAAGGATATACTTTTCAAAACAAACGATTTAAATTTTATGTTTAAAA |
| | TATAAACTTTAGATTTAAACTTTATTTAAATATCTGGTTCCTATGATTTTGACTT |
| | CAGTAAGTTCAAATAAAATATATTTTGCAATTCATTTTTACATTATAATTTAAAA |
| | AGAAGAAGCGATAAGTGGAGTCAGTTTCAATGCTAGGTGGGGTGGTTAATGAT |
| | TTTTCTGGTGTTGCTGCTAATGTGGATTAACAAATAAAAACATTCATTGCCTTTT |
| | G (SEQ ID NO: 127) |
| | >NP_006564.1 tumor necrosis factor ligand superfamily |
| | member 13B isoform 1 [Homo sapiens], amino acid sequence |
| | MDDSTEREQSRLTSCLKKREEMKLKECVSILPRKESPSVRSSKDGKLLAATLLLALL |
| | SCCLTVVSFYQVAALQGDLASLRAELQGHHAEKLPAGAGAPKAGLEEAPAVTAGL |
| | KIFEPPAPGEGNSSQNSRNKRAVQGPEETVTQDCLQLIADSETPTIQKGSYTFVP |
| | WLLSFKRGSALEEKENKILVKETGYFFIYGQVLYTDKTYAMGHLIQRKKVHV |
| | FGDELSLVTLFRCIQNMPETLPNNSCYSAGIAKLEEGDELQLAIPRENAQISLDG |
| | DVTFFGALKLL (SEQ ID NO: 128) |
| Human | >NM_003808.4 Homo sapiens TNF superfamily member 13 |
| APRIL | (TNFSF13), transcript variant alpha, mRNA, nucleic acid sequence |
| Bold: amino | GTCACTGGCAGCCCTGTCCTTCCTAGAGGGACTGGAACCTAATTCTCCTGAGGC |
| acids 111-250 | TGAGGGAGGGTGGAGGGTCTCAAGGCAACGCTGGCCCCACGACGGAGTGCCAG |
| | GAGCACTAACAGTACCCTTAGCTTGCTTTCCTCCTCCCTCCTTTTTATTTTCAAGT |
| | TCCTTTTTATTTCTCCTTGCGTAACAACCTTCTTCCCTTCTGCACCACTGCCCGTA |
| | CCCTTACCCGCCCCGCCACCTCCTTGCTACCCCACTCTTGAAACCACAGCTGTTG |
| | GCAGGGTCCCCAGCTCATGCCAGCCTCATCTCCTTTCTTGCTAGCCCCCAAAGG |
| | GCCTCCAGGCAACATGGGGGGCCCAGTCAGAGAGCCGGCACTCTCAGTTGCCC |
| | TCTGGTTGAGTTGGGGGGCAGCTCTGGGGGCCGTGGCTTGTGCCATGGCTCTGC |
| | TGACCCAACAAACAGAGCTGCAGAGCCTCAGGAGAGAGGTGAGCCGGCTGCAG |
| | GGGACAGGAGGCCCCTCCCAGAATGGGGAAGGGTATCCCTGGCAGAGTCTCCC |
| | GGAGCAGAGTTCCGATGCCCTGGAAGCCTGGGAGAATGGGGAGAGATCCCGGA |
| | AAAGGAGAGCAGTGCTCACCCAAAAACAGAAGAAGCAGCACTCTGTCCTGC |
| | ACCTGGTTCCCATTAACGCCACCTCCAAGGATGACTCCGATGTGACAGAGG |
| | TGATGTGGCAACCAGCTCTTAGGCGTGGGAGAGGCCTACAGGCCCAAGGA |
| | TATGGTGTCCGAATCCAGGATGCTGGAGTTTATCTGCTGTATAGCCAGGTC |
| | CTGTTTCAAGACGTGACTTTCACCATGGGTCAGGTGGTGTCTCGAGAAGGC |
| | CAAGGAAGGCAGGAGACTCTATTCCGATGTATAAGAAGTATGCCCTCCCAC |
| | CCGGACCGGGCCTACAACAGCTGCTATAGCGCAGGTGTCTTCCATTTACAC |
| | CAAGGGGATATTCTGAGTGTCATAATTCCCCGGGCAAGGGCGAAACTTAAC |
| | CTCTCTCCACATGGAACCTTCCTGGGGTTTGTGAAACTGTGATTGTGTTATA |
| | AAAAGTGGCTCCCAGCTTGGAAGACCAGGGTGGGTACATACTGGAGACAGCCA |
| | AGAGCTGAGTATATAAAGGAGAGGGAATGTGCAGGAACAGAGGCGTCTTCCTG |
| | GGTTTGGCTCCCCGTTCCTCACTTTTCCCTTTTCATTCCCACCCCCTAGACTTTGA |
| | TTTTACGGATATCTTGCTTCTGTTCCCCATGGAGCTCCGAATTCTTGCGTGTGTG |
| | TAGATGAGGGGCGGGGGACGGGCGCCAGGCATTGTCCAGACCTGGTCGGGGCC |
| | CACTGGAAGCATCCAGAACAGCACCACCATCTAGCGGCCGCTCGAGGGAAGCA |
| | CCCGCCGGTTGGCCGAAGTCCACGAAGCCGCCCTCTGCTAGGGAAAACCCCTG |
| | GTTCTCCATGCCACACCTCTCTCCAGGTGCCCTCTGCCTCTTCACCCCACAAGAA |
| | GCCTTATCCTACGTCCTTCTCTCCATCTATCGGACCCCAGTTTCCATCACTATCT |
| | CCAGAGATGTAGCTATTATGCGCCCGTCTACAGGGGGTGCCCGACGATGACGGT |
| | GCCTTCGCAGTCAAATTACTCTTCGGGTCCCAAGGTTTGGCTTTCACGCGCTCCA |
| | TTGCCCCGGCGTGGCAGGCCATTCCAAGCCCTTCCGGGCTGGAACTGGTGTCGG |
| | AGGAGCCTCGGGTGTATCGTACGCCCTGGTGTTGGTGTTGCCTCACTCCTCTGA |
| | GCTCTTCTTTCTGATCAAGCCCTGCTTAAAGTTAAATAAAATAGAATGAATGAT |
| | A (SEQ ID NO: 129) |
| | >NP_003799.1 tumor necrosis factor ligand superfamily member |
| | 13 isoform alpha precursor [Homo sapiens], amino acid sequence |
| | MPASSPFLLAPKGPPGNMGGPVREPALSVALWLSWGAALGAVACAMALLTQQTE |
| | LQSLRREVSRLQGTGGPSQNGEGYPWQSLPEQSSDALEAWENGERSRKRRAVLTQ |
| | KQKKQHSVLHLVPINATSKDDSDVTEVMWQPALRRGRGLQAQGYGVRIQDA |
| | GVYLLYSQVLFQDVTFTMGQVVSREGQGRQETLFRCIRSMPSHPDRAYNSCYS |
| | AGVFHLHQGDILSVIIPRARAKLNLSPHGTFLGFVKL (SEQ ID NO: 130) |
| Human | >NM_003809.3 Homo sapiens TNF superfamily member 12 |
| TWEAK | (TNFSF12), transcript variant 1, mRNA, nucleic acid sequence |
| Bold: amino | CTCTCCCCGGCCCGATCCGCCCGCCGGCTCCCCCTCCCCCGATCCCTCGGGTCCC |
| acids | GGGATGGGGGGGCGGTGAGGCAGGCACAGCCCCCCGCCCCCATGGCCGCCCGT |
| A95-H249 | CGGAGCCAGAGGCGGAGGGGGCGCCGGGGGGAGCCGGGCACCGCCCTGCTGG |
| | TCCCGCTCGCGCTGGGCCTGGGCCTGGCGCTGGCCTGCCTCGGCCTCCTGCTGG |
| | CCGTGGTCAGTTTGGGGAGCCGGGCATCGCTGTCCGCCCAGGAGCCTGCCCAGG |
| | AGGAGCTGGTGGCAGAGGAGGACCAGGACCCGTCGGAACTGAATCCCCAGACA |
| | GAAGAAAGCCAGGATCCTGCGCCTTTCCTGAACCGACTAGTTCGGCCTCGCAGA |
| | AGTGCACCTAAAGGCCGGAAAACACGGGCTCGAAGAGCGATCGCAGCCCA |
| | TTATGAAGTTCATCCACGACCTGGACAGGACGGAGCGCAGGCAGGTGTGG |
| | ACGGGACAGTGAGTGGCTGGGAGGAAGCCAGAATCAACAGCTCCAGCCCT |
| | CTGCGCTACAACCGCCAGATCGGGGAGTTTATAGTCACCCGGGCTGGGCT |
| | CTACTACCTGTACTGTCAGGTGCACTTTGATGAGGGGAAGGCTGTCTACCT |
| | GAAGCTGGACTTGCTGGTGGATGGTGTGCTGGCCCTGCGCTGCCTGGAGG |
| | AATTCTCAGCCACTGCGGCGAGTTCCCTCGGGCCCCAGCTCCGCCTCTGCC |
| | AGGTGTCTGGGCTGTTGGCCCTGCGGCCAGGGTCCTCCCTGCGGATCCGC |
| | ACCCTCCCCTGGGCCCATCTCAAGGCTGCCCCCTTCCTCACCTACTTCGGA |
| | CTCTTCCAGGTTCACTGAGGGGCCCTGGTCTCCCCGCAGTCGTCCCAGGCTGC |
| | CGGCTCCCCTCGACAGCTCTCTGGGCACCCGGTCCCCTCTGCCCCACCCTCAGC |
| | CGCTCTTTGCTCCAGACCTGCCCCTCCCTCTAGAGGCTGCCTGGGCCTGTTCACG |
| | TGTTTTCCATCCCACATAAATACAGTATTCCCACTCTTATCTTACAACTCCCCCA |
| | CCGCCCACTCTCCACCTCACTAGCTCCCCAATCCCTGACCCTTTGAGGCCCCCAG |
| | TGATCTCGACTCCCCCCTGGCCACAGACCCCCAGGGCATTGTGTTCACTGTACT |
| | CTGTGGGCAAGGATGGGTCCAGAAGACCCCACTTCAGGCACTAAGAGGGGCTG |
| | GACCTGGCGGCAGGAAGCCAAAGAGACTGGGCCTAGGCCAGGAGTTCCCAAAT |
| | GTGAGGGGCGAGAAACAAGACAAGCTCCTCCCTTGAGAATTCCCTGTGGATTTT |
| | TAAAACAGATATTATTTTTATTATTATTGTGACAAAATGTTGATAAATGGATATT |
| | AAATAGAA (SEQ ID NO: 131) |
| | >NP_003800.1 tumor necrosis factor ligand superfamily |
| | member 12 proprotein [Homo sapiens], amino acid sequence |
| | MAARRSQRRRGRRGEPGTALLVPLALGLGLALACLGLLLAVVSLGSRASLSAQEPA |
| | QEELVAEEDQDPSELNPQTEESQDPAPFLNRLVRPRRSAPKGRKTRARRAIAAHY |
| | EVHPRPGQDGAQAGVDGTVSGWEEARINSSSPLRYNRQIGEFIVTRAGLYYLY |
| | CQVHFDEGKAVYLKLDLLVDGVLALRCLEEFSATAASSLGPQLRLCQVSGLLA |
| | LRPGSSLRIRTLPWAHLKAAPFLTYFGLFQVH (SEQ ID NO: 132) |
| Human TNF | >NM_000594.4 Homo sapiens tumor necrosis factor (TNF), mRNA |
| alpha | AGCAGACGCTCCCTCAGCAAGGACAGCAGAGGACCAGCTAAGAGGGAGAGAA |
| Bold: amino | GCAACTACAGACCCCCCCTGAAAACAACCCTCAGACGCCACATCCCCTGACAA |
| acids | GCTGCCAGGCAGGTTCTCTTCCTCTCACATACTGACCCACGGCTCCACCCTCTCT |
| R78-L233 | CCCCTGGAAAGGACACCATGAGCACTGAAAGCATGATCCGGGACGTGGAGCTG |
| | GCCGAGGAGGCGCTCCCCAAGAAGACAGGGGGGCCCCAGGGCTCCAGGCGGTG |
| | CTTGTTCCTCAGCCTCTTCTCCTTCCTGATCGTGGCAGGCGCCACCACGCTCTTC |
| | TGCCTGCTGCACTTTGGAGTGATCGGCCCCCAGAGGGAAGAGTTCCCCAGGGAC |
| | CTCTCTCTAATCAGCCCTCTGGCCCAGGCAGTCAGATCATCTTCTCGAACCCC |
| | GAGTGACAAGCCTGTAGCCCATGTTGTAGCAAACCCTCAAGCTGAGGGGC |
| | AGCTCCAGTGGCTGAACCGCCGGGCCAATGCCCTCCTGGCCAATGGCGTG |
| | GAGCTGAGAGATAACCAGCTGGTGGTGCCATCAGAGGGCCTGTACCTCAT |
| | CTACTCCCAGGTCCTCTTCAAGGGCCAAGGCTGCCCCTCCACCCATGTGCT |
| | CCTCACCCACACCATCAGCCGCATCGCCGTCTCCTACCAGACCAAGGTCAA |
| | CCTCCTCTCTGCCATCAAGAGCCCCTGCCAGAGGGAGACCCCAGAGGGGG |
| | CTGAGGCCAAGCCCTGGTATGAGCCCATCTATCTGGGAGGGGTCTTCCAG |
| | CTGGAGAAGGGTGACCGACTCAGCGCTGAGATCAATCGGCCCGACTATCT |
| | CGACTTTGCCGAGTCTGGGCAGGTCTACTTTGGGATCATTGCCCTGTGAGG |
| | AGGACGAACATCCAACCTTCCCAAACGCCTCCCCTGCCCCAATCCCTTTATTAC |
| | CCCCTCCTTCAGACACCCTCAACCTCTTCTGGCTCAAAAAGAGAATTGGGGGCT |
| | TAGGGTCGGAACCCAAGCTTAGAACTTTAAGCAACAAGACCACCACTTCGAAA |
| | CCTGGGATTCAGGAATGTGTGGCCTGCACAGTGAAGTGCTGGCAACCACTAAG |
| | AATTCAAACTGGGGCCTCCAGAACTCACTGGGGCCTACAGCTTTGATCCCTGAC |
| | ATCTGGAATCTGGAGACCAGGGAGCCTTTGGTTCTGGCCAGAATGCTGCAGGAC |
| | TTGAGAAGACCTCACCTAGAAATTGACACAAGTGGACCTTAGGCCTTCCTCTCT |
| | CCAGATGTTTCCAGACTTCCTTGAGACACGGAGCCCAGCCCTCCCCATGGAGCC |
| | AGCTCCCTCTATTTATGTTTGCACTTGTGATTATTTATTATTTATTTATTATTTAT |
| | TTATTTACAGATGAATGTATTTATTTGGGAGACCGGGGTATCCTGGGGGACCCA |
| | ATGTAGGAGCTGCCTTGGCTCAGACATGTTTTCCGTGAAAACGGAGCTGAACAA |
| | TAGGCTGTTCCCATGTAGCCCCCTGGCCTCTGTGCCTTCTTTTGATTATGTTTTTT |
| | AAAATATTTATCTGATTAAGTTGTCTAAACAATGCTGATTTGGTGACCAACTGT |
| | CACTCATTGCTGAGCCTCTGCTCCCCAGGGGAGTTGTGTCTGTAATCGCCCTACT |
| | ATTCAGTGGCGAGAAATAAAGTTTGCTTAGAAAAGAAA (SEQ ID NO: 133) |
| | >NP_000585.2 tumor necrosis factor [Homo sapiens] |
| | MSTESMIRDVELAEEALPKKTGGPQGSRRCLFLSLFSFLIVAGATTLFCLLHFGVIGP |
| | QREEFPRDLSLISPLAQAVRSSSRTPSDKPVAHVVANPQAEGQLQWLNRRANAL |
| | LANGVELRDNQLVVPSEGLYLIYSQVLFKGQGCPSTHVLLTHTISRIAVSYQTK |
| | VNLLSAIKSPCQRETPEGAEAKPWYEPIYLGGVFQLEKGDRLSAEINRPDYLDF |
| | AESGQVYFGIIAL (SEQ ID NO: 134) |
| Human LTA | >NM_000595.4 Homo sapiens lymphotoxin alpha (LTA), |
| (TNFβ) | transcript variant 2, mRNA |
| Bold: amino | GCTCCGCACAGCAGGTGAGGCTCTCCTGCCCCATCTCCTTGGGCTGCCCGTGCT |
| acids | TCGTGCTTTGGACTACCGCCCAGCAGTGTCCTGCCCTCTGCCTGGGCCTCGGTCC |
| L35-L205 | CTCCTGCACCTGCTGCCTGGATCCCCGGCCTGCCTGGGCCTGGGCCTTGGTTCTC |
| | CCCATGACACCACCTGAACGTCTCTTCCTCCCAAGGGTGTGTGGCACCACCCTA |
| | CACCTCCTCCTTCTGGGGCTGCTGCTGGTTCTGCTGCCTGGGGCCCAGGGGCTC |
| | CCTGGTGTTGGCCTCACACCTTCAGCTGCCCAGACTGCCCGTCAGCACCCC |
| | AAGATGCATCTTGCCCACAGCACCCTCAAACCTGCTGCTCACCTCATTGGA |
| | GACCCCAGCAAGCAGAACTCACTGCTCTGGAGAGCAAACACGGACCGTGC |
| | CTTCCTCCAGGATGGTTTCTCCTTGAGCAACAATTCTCTCCTGGTCCCCAC |
| | CAGTGGCATCTACTTCGTCTACTCCCAGGTGGTCTTCTCTGGGAAAGCCTA |
| | CTCTCCCAAGGCCACCTCCTCCCCACTCTACCTGGCCCATGAGGTCCAGCT |
| | CTTCTCCTCCCAGTACCCCTTCCATGTGCCTCTCCTCAGCTCCCAGAAGAT |
| | GGTGTATCCAGGGCTGCAGGAACCCTGGCTGCACTCGATGTACCACGGGG |
| | CTGCGTTCCAGCTCACCCAGGGAGACCAGCTATCCACCCACACAGATGGC |
| | ATCCCCCACCTAGTCCTCAGCCCTAGTACTGTCTTCTTTGGAGCCTTCGCT |
| | CTGTAGAACTTGGAAAAATCCAGAAAGAAAAAATAATTGATTTCAAGACCTTC |
| | TCCCCATTCTGCCTCCATTCTGACCATTTCAGGGGTCGTCACCACCTCTCCTTTG |
| | GCCATTCCAACAGCTCAAGTCTTCCCTGATCAAGTCACCGGAGCTTTCAAAGAA |
| | GGAATTCTAGGCATCCCAGGGGACCACACCTCCCTGAACCATCCCTGATGTCTG |
| | TCTGGCTGAGGATTTCAAGCCTGCCTAGGAATTCCCAGCCCAAAGCTGTTGGTC |
| | TGTCCCACCAGCTAGGTGGGGCCTAGATCCACACACAGAGGAAGAGCAGGCAC |
| | ATGGAGGAGCTTGGGGGATGACTAGAGGCAGGGAGGGGACTATTTATGAAGGC |
| | AAAAAAATTAAATTATTTATTTATGGAGGATGGAGAGAGGGGAATAATAGAAG |
| | AACATCCAAGGAGAAACAGAGACAGGCCCAAGAGATGAAGAGTGAGAGGGCA |
| | TGCGCACAAGGCTGACCAAGAGAGAAAGAAGTAGGCATGAGGGATCACAGGG |
| | CCCCAGAAGGCAGGGAAAGGCTCTGAAAGCCAGCTGCCGACCAGAGCCCCACA |
| | CGGAGGCATCTGCACCCTCGATGAAGCCCAATAAACCTCTTTTCTCTGAAA |
| | (SEQ ID NO: 135) |
| | >NP_000586.2 lymphotoxin-alpha precursor [Homo sapiens] |
| | MTPPERLFLPRVCGTTLHLLLLGLLLVLLPGAQGLPGVGLTPSAAQTARQHPKMH |
| | LAHSTLKPAAHLIGDPSKQNSLLWRANTDRAFLQDGFSLSNNSLLVPTSGIYFV |
| | YSQVVFSGKAYSPKATSSPLYLAHEVQLFSSQYPFHVPLLSSQKMVYPGLQEP |
| | WLHSMYHGAAFQLTQGDQLSTHTDGIPHLVLSPSTVFFGAFAL (SEQ ID NO: |
| | 136) |
| Human LTB | >NM_002341.2 Homo sapiens lymphotoxin beta (LTB), |
| (TNFY) | transcript variant 1, mRNA |
| | AGTCTCAATGGGGGCACTGGGGCTGGAGGGCAGGGGTGGGAGGCTCCAGGGGA |
| | GGGGTTCCCTCCTGCTAGCTGTGGCAGGAGCCACTTCTCTGGTGACCTTGTTGCT |
| | GGCGGTGCCTATCACTGTCCTGGCTGTGCTGGCCTTAGTGCCCCAGGATCAGGG |
| | AGGACTGGTAACGGAGACGGCCGACCCCGGGGCACAGGCCCAGCAAGGACTG |
| | GGGTTTCAGAAGCTGCCAGAGGAGGAGCCAGAAACAGATCTCAGCCCCGGGCT |
| | CCCAGCTGCCCACCTCATAGGCGCTCCGCTGAAGGGGCAGGGGCTAGGCTGGG |
| | AGACGACGAAGGAACAGGCGTTTCTGACGAGCGGGACGCAGTTCTCGGACGCC |
| | GAGGGGCTGGCGCTCCCGCAGGACGGCCTCTATTACCTCTACTGTCTCGTCGGC |
| | TACCGGGGCCGGGCGCCCCCTGGCGGCGGGGACCCCCAGGGCCGCTCGGTCAC |
| | GCTGCGCAGCTCTCTGTACCGGGCGGGGGGCGCCTACGGGCCGGGCACTCCCG |
| | AGCTGCTGCTCGAGGGCGCCGAGACGGTGACTCCAGTGCTGGACCCGGCCAGG |
| | AGACAAGGGTACGGGCCTCTCTGGTACACGAGCGTGGGGTTCGGCGGCCTGGT |
| | GCAGCTCCGGAGGGGCGAGAGGGTGTACGTCAACATCAGTCACCCCGATATGG |
| | TGGACTTCGCGAGAGGGAAGACCTTCTTTGGGGCCGTGATGGTGGGGTGAGGG |
| | AATATGAGTGCGTGGTGCGAGTGCGTGAATATTGGGGGCCCGGACGCCCAGGA |
| | CCCCATGGCAGTGGGAAAAATGTAGGAGACTGTTTGGAAATTGATTTTGAACCT |
| | GATGAAAATAAAGAATGGAAAGCTTCAGTGCTGCCGATAAA (SEQ ID NO: 137) |
| | >NP_002332.1 lymphotoxin-beta isoform a [Homo sapiens] |
| | MGALGLEGRGGRLQGRGSLLLAVAGATSLVTLLLAVPITVLAVLALVPQDQGGLV |
| | TETADPGAQAQQGLGFQKLPEEEPETDLSPGLPAAHLIGAPLKGQGLGWETTKEQA |
| | FLTSGTQFSDAEGLALPQDGLYYLYCLVGYRGRAPPGGGDPQGRSVTLRSSLYRAG |
| | GAYGPGTPELLLEGAETVTPVLDPARRQGYGPLWYTSVGFGGLVQLRRGERVYVN |
| | ISHPDMVDFARGKTFFGAVMVG (SEQ ID NO: 138) |
| Human | >NM_001399.5 Homo sapiens ectodysplasin A (EDA), |
| Ectodysplasin | transcript variant 1, mRNA |
| A | CCCTTTCCCACCCCTCGGAGTAGAGCTGCACATGCGGCTGCTCCCTGCTCCGTCC |
| | CGCCCAGCCACTGTCGCGCAGGAACGGGTCCCTGCAGCCCCCAGCCGATGGCA |
| | GGACAGTAGCCGCCTGTCAGAGGTCGTGAACGGCTGAGGCAGACGCAGCGGCT |
| | CCCGGGCCTCAAGAGAGTGGGTGTCTCCGGAGGCCATGGGCTACCCGGAGGTG |
| | GAGCGCAGGGAACTCCTGCCTGCAGCAGCGCCGCGGGAGCGAGGGAGCCAGG |
| | GCTGCGGGTGTGGCGGGGCCCCTGCCCGGGGGGCGAAGGGAACAGCTGCCTG |
| | CTCTTCCTGGGTTTCTTTGGCCTCTCGCTGGCCCTCCACCTGCTGACGTTGTGCT |
| | GCTACCTAGAGTTGCGCTCGGAGTTGCGGCGGGAACGTGGAGCCGAGTCCCGC |
| | CTTGGCGGCTCGGGCACCCCTGGCACCTCTGGCACCCTAAGCAGCCTCGGTGGC |
| | CTCGACCCTGACAGCCCCATCACCAGTCACCTTGGGCAGCCGTCACCTAAGCAG |
| | CAGCCATTGGAACCGGGAGAAGCCGCACTCCACTCTGACTCCCAGGACGGGCA |
| | CCAGATGGCCCTATTGAATTTCTTCTTCCCTGATGAAAAGCCATACTCTGAAGA |
| | AGAAAGTAGGCGTGTTCGCCGCAATAAAAGAAGCAAAAGCAATGAAGGAGCA |
| | GATGGCCCAGTTAAAAACAAGAAAAAGGGAAAGAAAGCAGGACCTCCTGGAC |
| | CCAATGGCCCTCCAGGACCCCCAGGACCTCCAGGACCCCAGGGACCCCCAGGA |
| | ATTCCAGGGATTCCTGGAATTCCAGGAACAACTGTTATGGGACCACCTGGTCCT |
| | CCAGGTCCTCCTGGTCCTCAAGGACCCCCTGGCCTCCAGGGACCTTCTGGTGCT |
| | GCTGATAAAGCTGGAACTCGAGAAAACCAGCCAGCTGTGGTGCATCTACAGGG |
| | CCAAGGGTCAGCAATTCAAGTCAAGAATGATCTTTCAGGTGGAGTGCTCAATGA |
| | CTGGTCTCGCATCACTATGAACCCCAAGGTGTTTAAGCTACATCCCCGCAGCGG |
| | GGAGCTGGAGGTACTGGTGGACGGCACCTACTTCATCTATAGTCAGGTAGAAGT |
| | ATACTACATCAACTTCACTGACTTTGCCAGCTATGAGGTGGTGGTGGATGAGAA |
| | GCCCTTCCTGCAGTGCACACGCAGCATCGAGACGGGCAAGACCAACTACAACA |
| | CTTGCTATACCGCAGGCGTCTGCCTCCTCAAGGCCCGGCAGAAGATCGCCGTCA |
| | AGATGGTGCACGCTGACATCTCCATCAACATGAGCAAGCACACCACGTTCTTTG |
| | GGGCCATCAGGCTGGGTGAAGCCCCTGCATCCTAGATTCCCCCCATTTTGCCTC |
| | TGTCCGTGCCCCTTCCCTGGGTTTGGGAGCCAGGACTCCCAGAACCTCTAAGTG |
| | CTGCTGTGGAGTGAGGTGTATTGGTGTTGCAGCCGCAGAGAAATGCCCCAGTGT |
| | TATTTATTCCCCAGTGACTCCAGGGTGACAAGGCCTGCTTGACTTTCCAGAATG |
| | ACCTTGAGTTAACAGGACAGTTGATGGAGCCCCAGGGTTTACATGAAGCAGAA |
| | CCTTCTTTGGTTCCATGTTGACTGACTTATGGCATGACTCTTCAACCCCGAGGTC |
| | CCTGTTGTCAGATCTATTGTTTGTTGCACTAAAATGAGGATCCAGGGCAGCAGG |
| | CCAGAGAAAGCAAAGGTGCACTCCAGACTCTGGGGGTGGACATCTGACCCCAA |
| | GGGGGCTGCTGCTCCTCTCTTGGGTAGGGTAGTGGCTGGGGTGGAGTGGGAAG |
| | GGAGCATTGCAGCCTAAGAAGAAGGCCAGAGAGGGAAAAGGCAGGTGCTTTTG |
| | GCAGAGACCATAAGAGAAACCTGCCAAGGAGCATCCTTGGCAGTGGGAATGTT |
| | CTTTCTGCTCTATACTGTGGCCTGCAGGAGGGTTGGAGTGCTCTTCCCACTCCAG |
| | CTGACAGCCACACCGTGGCAGCTTGCTGGGCTTTGGGAAGTTTGCTGTGCTTTG |
| | GAACAATCACAGGGAATGGCCACAAACCTGCCCGCCTAAGACCCTGAATCCGT |
| | ACTTGGGTCACATGACTCTCATTTTATTTACAGCTGTGCTCCACACTCAGAAAAT |
| | TCCCTGGGGTCACCTTCTAGTTGCCCCCATTCCCAGCCTGACTAGAACTCCTGTC |
| | TTCTTTCTCCATGGAGCCTACCTCTGTCTGAGACAGGTGCCTAACCTGGGACCTG |
| | TGGTCATGTGAGTCTGGGATATTCTTTAGCTTACCTGGGCACAAACAGAATTTT |
| | CCATTTATTAAGCAGTACAAATGTTTTTCATCCATTCCTAATCAAATTCTGTCTG |
| | GGGACGAAGGGTTGGACGGGATGACCTCCAGAAGTCCCTTCAATTTCTAGTACC |
| | TGTGACTCTTAGCCCTCACCACAGCCTTCTAAATTCCCAAATCCTAGACTGCTCC |
| | TGGGCATTAGCAAGGCAGAGCCTTTTTACCTGGCCTAGAAAGGGCAAGGGGTG |
| | AGGATAGGACAGAGGGATTTTGTTCAAGTTTGCTGCAACCCAAGTGGACGTTAG |
| | GCCAGGCCTTATCTGAAAGGCCAGCAGCTGATGCTGTACTAACCCAGTCTTTCT |
| | TCACTCTGGCTTCAAAAAGCCACAGCAGAGCATTGTCACCGCAGGTGCTCATGC |
| | TGCTCCCCTAAAGCCAGGCTCAGGAGAAGCCAGTGTCTAGGCACTGAGCAGGG |
| | ATCTGCCCCCTAGTTCAGGTCCAAATTCACCTTCCCCTAAACCCCAAGCTTCCCA |
| | ACAGATCATATGGTAGGACCCTCGAGAGCCTTACTTCAAAGTGCCTGGGCTCAG |
| | CCTGGTTTCTGGGTGCTAGATCCAGCCCAAACCTGGGAAGGCCAGCCTTGTACA |
| | GTCTGCTCCTCTTGTTCCTGAAATGTGTTTCCTTTTCAGGAGATGGGGAATAATT |
| | TCCTTCAGGCAGCTGAAATTCACCAAGAACAGCGGGTACTTATTTCTCAGCTGT |
| | GCCTTCCCTTTCTAAGCAACCACACTGCTTGGCCCTTCAAGGGTCAGGGTGAGA |
| | CGTGATGGGCTAGGCCTCCGTTGTCTGGTTGCTAATGACAGCCTTGCAACCCAA |
| | GGTGAGGTGAACTCCAGGCATGTGTCTGGCCCTAACTCCTATAAAGTGCCTCGG |
| | ACAGTCCGCAGTTGTAGCAGAAACCAACAAGAACCACTCCTTCATGTTTGGAAA |
| | ATAATTTCTCTTGTATTATCTCCTTTGAAGAAGGCAAGGCTGATAATATGACAA |
| | ACATCATTGTTTAGATGAGGCTCAGAGAGGTAGCACTCTCAGAGTGTTTTGACC |
| | AGTTTAAGCCGCAGACCTGGAGCTTCAGCCAGGTCTGACTCCAAAGCTGTTCCA |
| | TTACACCACAGCATTGTGTGGAATTTGAGGTCTAGAGAGAACCAATAAAAGTG |
| | GTAATTGGGAACTGAAATCCTTGAGAGTTCCGGGGAGAAACCCAGAGATGCCT |
| | GATTTCATTCCTCGATGGTAATACCCGTCCTCTCGGCTGCCAGGGGCTCTGTGGC |
| | AAAAAGAGTCAGACATTTCTTTGGAAAACAGCGAACAGCCTTAGAGCTCTTGTG |
| | TTCAGAAGAATCTTCCTGGCACAATGTTGGAGCAGCAGGCCTCTGGGACCCACA |
| | GAACTTGTGGCCTTTATGTTCTTTCACCCATCCTAGGAACCAGCCAACCATCATG |
| | TGTAGAGCCCCTACTGTGGGCAAAGTCCTCCTTTCATTACCCTACAGACAGCTT |
| | ACAGGAGCCAGCCTGCTTCCCACAACTACTAGTGTGACTCCTTATCTCTTTCCAC |
| | CATACCTTAGAGACTTTGATACTACCAGGGTCTCTCAGGGATGGAGGGAAGACC |
| | TGAAAGAGAGGACTGGTTCTGAGGCCAGAAAGGTGTGAGGAGAGAGGAGGAA |
| | AAGTCTTCCTAATTGTGCCCCTAAAGAGCATCCTGATACCATTCTATTCTCCAGA |
| | CATGGAGGGGATGATAAAGGAAATAGGATCTCCACTGGACCCTTGATTCATTCT |
| | GAACCCTCCAAAGGAACTCTAGAGGGCGAGGGATGATGAGGGAAGCAATAGGT |
| | AGCTGGGGAGCCCTATTGCTGCTAAGTCATTGGCAAAGTGACAAAGCAATTTAC |
| | TGATGAGAGAATGTGGAAATAGATGTGCAGTTTGGAATTATGTTGGTGTGAATT |
| | TGCCAGAGGACCAATGCTTGCATGGAGAATGGGACGAGGACATTTGTGGGCAA |
| | GCAGATGACAGAGGTTTGAAGGAGAATGGCATGGCAGGAGTCTCTGCCAGTTA |
| | CTTGGGCTTCAACAGCCAAGCTGGCACAAAAGACAGCTGGCGGAGGCTGCTCG |
| | GCTACTGGTTACCTGGAGAAGTAGTATTTGCCTATTTCCCCCTTCATCCATCCTG |
| | AGCCAAATTTCTTTTGCTGAACAGGAAAGAGCTAGGAACCCTGGAGGTAAACA |
| | AAGACTTTGATCCATGTATGAGTGTATGTGTTTATGTAACTTCCTGTGGATGCAA |
| | ATAGATTCAGAGAAATTTAGAGCTAAAAAGGCCCTTAGAGGGAATCTAGCCCA |
| | ACCTACATTCCACCCTGTTACTTATGTAGAAACTGAGGCCCAGAGAGGGAAGAT |
| | GACCTGCCCCAAGTGGTGAGCAAGCACCAACCTCCAGACTCAGCAGAGTGAGG |
| | GGGTAAAGCAGTTCCTGTCCCACATGGCCATCTTCTTTCTTCCACCCACAAACTC |
| | CAGGCTGGAAGTACTTGGCCCCCTTCAGGAGCCTGGCCAGGCAGGGAGAGAGT |
| | AGCTGCAGCCTTCATCAGAACTCTTCCTCCTCCCAAGGCATTCTCCCAGCTCTAG |
| | CCTCTGGACTGGAAAGCACAAGACTGGCCCAGTGCCAGCAAGTCCTTAGGCTA |
| | CTGTAATGCTGCCTCAGGACCCATCCCTGCCTGGAGGCTCCTCTAGGCCCTGTG |
| | AGCACAAAGAAGAAAGCTGATTTTTGTCTTTTAATCCATTTCAGGACTCTCTCC |
| | AGGAGGGCTCGGGGTGTGTCATTTCTATATTCCTCCAGCTGGGATTGGGGGGTG |
| | GGCTTTGTTGTGAGAATGGCCTGGAGCAGGCCCAATGCTGCTTTTGGGGGTCAG |
| | CATCCAGTGTGAGATACTGTGTATATAAACTATATATAATGTATATAAACTGGG |
| | ATGTAAGTTTGTGTAAATTAATGGTTTATTCTTTGCAAATAAAACGCTTTCCCCG |
| | TCTGTTCTTGAAA (SEQ ID NO: 139) |
| | >NP_001390.1 ectodysplasin-A isoform 1 [Homo sapiens] |
| | MGYPEVERRELLPAAAPRERGSQGCGCGGAPARAGEGNSCLLFLGFFGLSLALHLL |
| | TLCCYLELRSELRRERGAESRLGGSGTPGTSGTLSSLGGLDPDSPITSHLGQPSPKQQ |
| | PLEPGEAALHSDSQDGHQMALLNFFFPDEKPYSEEESRRVRRNKRSKSNEGADGPV |
| | KNKKKGKKAGPPGPNGPPGPPGPPGPQGPPGIPGIPGIPGTTVMGPPGPPGPPGPQGP |
| | PGLQGPSGAADKAGTRENQPAVVHLQGQGSAIQVKNDLSGGVLNDWSRITMNPK |
| | VFKLHPRSGELEVLVDGTYFIYSQVEVYYINFTDFASYEVVVDEKPFLQCTRSIETG |
| | KTNYNTCYTAGVCLLKARQKIAVKMVHADISINMSKHTTFFGAIRLGEAPAS (SEQ |
| | ID NO: 140) |

The polypeptides provided in Table 3A and 3B above are involved in a range of biological processes, including but not limited to, suppressing the adaptive arm of the immune system (e.g., PD-L1); cellular adhesion (e.g., nectin), immune activation (e.g., HVEM), and the like. The POI domains can also be used to track, purify, or identify the engineered EVs from native EVs (e.g., mScarlet and nanoluciferase). The genes, transcripts, polypeptides, variants, and fragments thereof can be used in any combination from Table 3A and 3B to be displayed by an engineered EV provided herein. In some embodiments, the POI domain is the human polypeptide. In some embodiments, the POI domain is a homologue of the human polypeptide (e.g., mouse).

In some embodiments of any of the aspects, the engineered cell or EV provided herein comprises an exogenous nucleic acid encoding one or more exogenous polypeptide(s) selected from the group consisting of: the polypeptides listed in Table 3A and 3B.

In some embodiments of any of the aspects, the POI domain is a type I membrane protein or a fragment thereof. In some embodiments of any of the aspects, the POI domain is a type II membrane protein or a fragment thereof. In some embodiments of any of the aspects, the POI domain is a secreted protein or a fragment thereof. In some embodiments of any of the aspects, the POI domain is a type III membrane protein or a fragment thereof. In some embodiments of any of the aspects, the POI domain is multipass membrane protein or a fragment thereof. In some embodiments of any of the aspects, the POI domain is a cytosolic protein or a fragment thereof. In some embodiments of any of the aspects, the POI domain is a lipid linked membrane protein or a fragment thereof. In some embodiments of any of the aspects, the POI domain is a GPI membrane linked protein or a fragment thereof.

In some embodiments of any of the aspects, the POI domain is PD-L1 or a fragment thereof. In some embodiments of any of the aspects, the POI domain is PD-L2 or a fragment thereof. In some embodiments of any of the aspects, the POI domain is FGL1 or a fragment thereof. In some embodiments of any of the aspects, the POI domain is TSG-6 or a fragment thereof. In some embodiments of any of the aspects, the POI domain is CTLA-4 or a fragment thereof. In some embodiments of any of the aspects, the POI domain is IL-10 or a fragment thereof. In some embodiments of any of the aspects, the POI domain is IL-2 or a fragment thereof. In some embodiments of any of the aspects, the POI domain is IL-12 or a fragment thereof. In some embodiments of any of the aspects, the POI domain is B7-H7 or a fragment thereof. In some embodiments of any of the aspects, the POI domain is VISG8 or a fragment thereof. In some embodiments of any of the aspects, the POI domain is VISG3 or a fragment thereof. In some embodiments of any of the aspects, the POI domain is VISG4 or a fragment thereof. In some embodiments of any of the aspects, the POI domain is TIM-4 or a fragment thereof. In some embodiments of any of the aspects, the POI domain is BTN3A1 or a fragment thereof. In some embodiments of any of the aspects, the POI domain is BTN2A1 or a fragment thereof. In some embodiments of any of the aspects, the POI domain is BTN1A1 or a fragment thereof. In some embodiments of any of the aspects, the POI domain is VISTA or a fragment thereof. In some embodiments of any of the aspects, the POI domain is B7-H3 or a fragment thereof. In some embodiments of any of the aspects, the POI domain is VTCN1 or a fragment thereof. In some embodiments of any of the aspects, the POI domain is HVEM or a fragment thereof. In some embodiments of any of the aspects, the POI domain is Galectin-9 or a fragment thereof. In some embodiments of any of the aspects, the POI domain is CEACAM1 or a fragment thereof. In some embodiments of any of the aspects, the POI domain is OX-2 or a fragment thereof. In some embodiments of any of the aspects, the POI domain is PVR or a fragment thereof. In some embodiments of any of the aspects, the POI domain is Nectin-2 or a fragment thereof. In some embodiments of any of the aspects, the POI domain is Tim-3 or a fragment thereof. In some embodiments of any of the aspects, the POI domain is Tim-4 or a fragment thereof. In some embodiments of any of the aspects, the POI domain is TIGIT or a fragment thereof. In some embodiments of any of the aspects, the POI domain is CD27 or a fragment thereof. In some embodiments of any of the aspects, the POI domain is CD28 or a fragment thereof. In some embodiments of any of the aspects, the POI domain is CD155 or a fragment thereof. In some embodiments of any of the aspects, the POI domain is LAG3 or a fragment thereof. In some embodiments of any of the aspects, the POI domain is CD158 or a fragment thereof. In some embodiments of any of the aspects, the POI domain is CD80 or a fragment thereof. In some embodiments of any of the aspects, the POI domain is CD86 or a fragment thereof. In some embodiments of any of the aspects, the POI domain is CD226 or a fragment thereof.

In some embodiments of any of the aspects, the POI domain is 4-1BBL or a fragment thereof. In some embodiments of any of the aspects, the POI domain is OX40L or a fragment thereof. In some embodiments of any of the aspects, the POI domain is GITRL or a fragment thereof. In some embodiments of any of the aspects, the POI domain is CD27L or a fragment thereof. In some embodiments of any of the aspects, the POI domain is CD30L or a fragment thereof. In some embodiments of any of the aspects, the POI domain is LIGHT or a fragment thereof. In some embodiments of any of the aspects, the POI domain is TRAIL or a fragment thereof. In some embodiments of any of the aspects, the POI domain is RANKL or a fragment thereof. In some embodiments of any of the aspects, the POI domain is TL1A or a fragment thereof. In some embodiments of any of the aspects, the POI domain is FASL or a fragment thereof. In some embodiments of any of the aspects, the POI domain is BAFF or a fragment thereof. In some embodiments of any of the aspects, the POI domain is APRIL or a fragment thereof. In some embodiments of any of the aspects, the POI domain is TWEAK or a fragment thereof. In some embodiments of any of the aspects, the POI domain is CD40L or a fragment thereof. In some embodiments of any of the aspects, the POI domain is Tumor Necrosis Factor (TNFα) or a fragment thereof. In some embodiments of any of the aspects, the POI domain is Lymphotoxin alpha (LTA, i.e., TNFβ) or a fragment thereof. In some embodiments of any of the aspects, the POI domain is Lymphotoxin beta (LTB, i.e., TNFγ) or a fragment thereof. In some embodiments of any of the aspects, the POI domain is Ectodysplasin A or a fragment thereof.

### Target Protein

The terms “target protein” and “target receptor” and “target molecule” refer to a protein or a molecule that binds to a protein of interest (POI) or fusion polypeptide.

In some embodiments of any of the aspects, the POI domain substantially binds to one or more of a target protein. In some embodiments of any of the aspects, the fusion polypeptide substantially binds to one or more of a target protein. In some embodiments of any of the aspects, fusion polypeptide effector domain substantially binds to one or more of a target protein. In some embodiments of any of the aspects, the fusion polypeptide agonistic muti-effector domain substantially binds to one or more of a target protein. In some embodiments of any of the aspects, one or more fusion polypeptide substantially binds to one or more of a target protein. It is understood that in some embodiments of any of the aspects, the fusion polypeptide may be displayed on an extracellular vesicle as described herein. In some embodiments of any of the aspects, the target protein is on a cell. In some embodiments of any of the aspects, the target protein is on a eukaryotic cell. In some embodiments of any of the aspects, the target protein is on an animal cell. In some embodiments of any of the aspects, the target protein is on a human cell. In some embodiments of any of the aspects, the target protein is on an animal cell other than a human cell. In some embodiments of any of the aspects, the target protein is a cellular receptor. In some embodiments of any of the aspects, the target protein is an immunosuppressive polypeptide. In some embodiments of any of the aspects, the target protein is an immunostimulatory polypeptide. In some embodiments of any of the aspects, the target protein is type I membrane protein. In some embodiments of any of the aspects, the target protein is a type II membrane protein. In some embodiments of any of the aspects, the target protein is a type III membrane protein. In some embodiments of any of the aspects, the target protein is a GPI linked membrane protein. In some embodiments of any of the aspects, the target protein is a secreted protein. In some embodiments of any of the aspects, the target protein is a tetraspanin. In some embodiments of any of the aspects, the target protein is an interleukin. In some embodiments of any of the aspects, the target protein is found on the surface of a cell. In some embodiments of any of the aspects, the target protein is a cluster of differentiation protein. In some embodiments of any of the aspects, the target protein is on cells associated with the immune system. In some embodiments of any of the aspects, the target protein is on an immune cell. In some embodiments of any of the aspects, the target protein is on a stem cell. In some embodiments of any of the aspects, the target protein is on a hematopoietic stem cell. In some embodiments of any of the aspects, the target protein is on leukocytes. In some embodiments of any of the aspects, the target protein is on a myeloid cell. In some embodiments of any of the aspects, the target protein is on a lymphoid stem cell. In some embodiments of any of the aspects, the target protein is on monocyte cells. In some embodiments of any of the aspects, the target protein is on lymphoid progenitor cells. In some embodiments of any of the aspects, the target protein is on lymphocytes. In some embodiments of any of the aspects, the target protein is on T-cells. In some embodiments of any of the aspects, the target protein is on effector T cells. In some embodiments of any of the aspects, the target protein is on CD4+ T-cells. In some embodiments of any of the aspects, the target protein is on CD8+ T-cells. In some embodiments of any of the aspects, the target protein is on Treg cells. In some embodiments of any of the aspects, the target protein is on Th17 cells. In some embodiments of any of the aspects, the target protein is on Th2 cells. In some embodiments of any of the aspects, the target protein is on Th1 cells. In some embodiments of any of the aspects, the target protein is on Th0 cells. In some embodiments of any of the aspects, the target protein is on gamma delta T cells. In some embodiments of any of the aspects, the target protein is on tissue-resident memory T cells. In some embodiments of any of the aspects, the target protein is on B-cells. In some embodiments of any of the aspects, the target protein is on B-cells. In some embodiments of any of the aspects, the target protein is on plasma B-cells. In some embodiments of any of the aspects, the target protein is on Natural Killer cells. In some embodiments of any of the aspects, the target protein is on an erythroid progenitor cell. In some embodiments of any of the aspects, the target protein is on a megakaryocyte cell. In some embodiments of any of the aspects, the target protein is on an erythroblast cell. In some embodiments of any of the aspects, the target protein is on a platelet cell. In some embodiments of any of the aspects, the target protein is on an erythrocyte cell. In some embodiments of any of the aspects, the target protein is on myeloid progenitor cells. In some embodiments of any of the aspects, the target protein is on myeloblast cells. In some embodiments of any of the aspects, the target protein is on monocyte cells. In some embodiments of any of the aspects, the target protein is on macrophage cells. In some embodiments of any of the aspects, the target protein is on dendritic cells. In some embodiments of any of the aspects, the target protein is on granulocyte cells. In some embodiments of any of the aspects, the target protein is on eosinophil cells. In some embodiments of any of the aspects, the target protein is on neutrophil cells. In some embodiments of any of the aspects, the target protein is on basophil cells. In some embodiments of any of the aspects, the target protein is on mast cells. In some embodiments of any of the aspects, the target protein is on dendritic cells. In some embodiments of any of the aspects, the target protein is on an antigen presenting cell. In some embodiments of any of the aspects, the target protein is on a cancer cell. In some embodiments of any of the aspects, the target protein is on an epithelial cell. In some embodiments of any of the aspects, the target protein is on a muscle cell. In some embodiments of any of the aspects, the target protein is on a cardiac cell. In some embodiments of any of the aspects, the target protein is on a skeletal muscle cell. In some embodiments of any of the aspects, the target protein is on a type I skeletal muscle cell. In some embodiments of any of the aspects, the target protein is on a type II skeletal muscle cell. In some embodiments of any of the aspects, the target protein is on a smooth muscle cell. In some embodiments of any of the aspects, the target protein is on a nervous tissue cell. In some embodiments of any of the aspects, the target protein is on a neuron cell. In some embodiments of any of the aspects, the target protein is on a sensory neuron cell. In some embodiments of any of the aspects, the target protein is on a motor neuron cell. In some embodiments of any of the aspects, the target protein is on a microglial cell. In some embodiments of any of the aspects, the target protein is on an astrocyte cell. In some embodiments of any of the aspects, the target protein is on a neuroendocrine cell. In some embodiments of any of the aspects, the target protein is on an oligodendrocyte cell. In some embodiments of any of the aspects, the target protein is on a Schwann cell. In some embodiments of any of the aspects, the target protein is on an endothelial cell. In some embodiments of any of the aspects, the target protein is on an epithelial cell. In some embodiments of any of the aspects, the target protein is on a hepatocyte cell. In some embodiments of any of the aspects, the target protein is on a hepatic stellate cell. In some embodiments of any of the aspects, the target protein is on a hepatic sinusoidal endothelial cell. In some embodiments of any of the aspects, the target protein is on a Kupffer cell. In some embodiments of any of the aspects, the target protein is on an epithelial cell. In some embodiments of any of the aspects, the target protein is on a spleen tissue cell. In some embodiments of any of the aspects, the target protein is on an enterocyte cell. In some embodiments of any of the aspects, the target protein is on an intestinal epithelium cell. In some embodiments of any of the aspects, the target protein is on a goblet cell. In some embodiments of any of the aspects, the target protein is on a Paneth cell. In some embodiments of any of the aspects, the target protein is on a pancreas cell. In some embodiments of any of the aspects, the target protein is on a pancreatic cell, for example alpha, beta, delta, epsilon and gamma cells. In some embodiments of any of the aspects, the target protein is on a pancreatic islet of Langerhans cell. In some embodiments of any of the aspects, the target protein is on an astrocyte cell. In some embodiments of any of the aspects, the target protein is on a lung cell. In some embodiments of any of the aspects, the target protein is on an alveolar cell. In some embodiments of any of the aspects, the target protein is on a bronchiolar cell. In some embodiments of any of the aspects, the target protein is on a retinal cell. In some embodiments of any of the aspects, the target protein is on a photoreceptor cell. In some embodiments of any of the aspects, the target protein is on a rod cell. In some embodiments of any of the aspects, the target protein is on a cone cell. In some embodiments of any of the aspects, the target protein is on a retinal ganglionic cell. In some embodiments of any of the aspects, the target protein is on a retinal bipolar cell. In some embodiments of any of the aspects, the target protein is on a retinal horizontal cell. In some embodiments of any of the aspects, the target protein is on a retinal Muller cell. In some embodiments of any of the aspects, the target protein is on a retinal amacrine cell. In some embodiments of any of the aspects, the target protein is on a cornea cell. In some embodiments of any of the aspects, the target protein is on a pupil cell. In some embodiments of any of the aspects, the target protein is on an iris cell. In some embodiments of any of the aspects, the target protein is on a sclera cell. In some embodiments of any of the aspects, the target protein is on a skin cell. In some embodiments of any of the aspects, the target protein is on a keratinocyte cell. In some embodiments of any of the aspects, the target protein is on a melanocyte cell. In some embodiments of any of the aspects, the target protein is on a Merkel cell. In some embodiments of any of the aspects, the target protein is on a Langerhans cell. In some embodiments of any of the aspects, the target protein is on connective tissue cell. In some embodiments of any of the aspects, the target protein is on fibroblast cell. In some embodiments of any of the aspects, the target protein is on an adipose cell. In some embodiments of any of the aspects, the target protein is on a muscle fiber cell.

The engineered extracellular vesicles provided herein can be designed to activate, block, agonize, antagonize, or modulate a given target protein with the appropriate POI domain, e.g., a POI signaling domain, that binds to or modulates the function or expression of the target protein. The engineered extracellular vesicles provided herein can display a fusion polypeptide wherein the fusion polypeptide is designed to activate, block, agonize, antagonize, or modulate a given target protein with the appropriate POI domain, e.g., a POI signaling domain, that binds to, activates, blocks, agonizes, antagonizes, or modulates the function or expression of the target protein. The engineered extracellular vesicles provided herein can be designed to comprise at least one fusion polypeptide wherein the fusion polypeptide further comprises an appropriate POI domain, wherein the POI domain binds to, activates, blocks, agonizes, antagonizes, or modulates the function, activation, suppression, or expression of a target protein. The engineered extracellular vesicles provided herein can be designed to comprise at least one fusion polypeptide wherein the fusion polypeptide further comprises an appropriate POI domain, wherein the POI domain binds to or modulates the function or expression of the target protein. The engineered extracellular vesicles provided herein can be designed to comprise at least one fusion polypeptide wherein the fusion polypeptide further comprises an appropriate POI domain, wherein the POI domain activates the function or expression of the target protein. The engineered extracellular vesicles provided herein can be designed to comprise at least one fusion polypeptide wherein the fusion polypeptide further comprises an appropriate POI domain, wherein the POI domain induces the function or expression of the target protein. The engineered extracellular vesicles provided herein can be designed to comprise at least one fusion polypeptide wherein the fusion polypeptide further comprises an appropriate POI domain, wherein the POI domain suppresses the function or expression of the target protein. Non-limiting examples of target proteins include those listed in Table 4 (below).

TABLE 4
Exemplary Target Proteins

| | | | |
|---|---|---|---|
| PD-1 | VISTA | LAG-3 | CD44 |
| CD80 | BTLA | CD112 | IL10RA |
| CD86 | CD160 | CD200R | IL10RB |
| CD28 | HVEM | CD200 | Tim-4 |
| ICOS | CD2 | Galectin 9 | TNFRSF25 |
| CD28H | SLAM CD150 | TIM-3 | TNFRSF6B |
| PD-L1 | CD58 | CD226 | CD113 |
| CTLA-4 | TIM-1 | CD155 | CD27 |
| 4-1BB (CD137) | TIM-4 | CD122 | CD30 |
| GITR | CD40 | DR3 | TIGIT |
| CD27L (CD70) | CD30L | GITRL | CD40L |
| CD48 | CD244 | DcR3 | CD28H |
| LFA-3 (CD58) | CD98 | PD-L2 | Nectin |
| Butyrophilin | TNF Receptor | TNF receptor | TIM |
| family | Superfamily | associated factor | family |
| members | members | (TRAF) family | members |
| | | members | |
| B7/CD28 family | SLAM family | Nectin-like | Collagen |
| members | members | binding receptors | family proteins |
| LAIR-1 (CD305) | 4-1BBL | OX40 | OX40L |
| TNFR1 | TNFR2 | BCMA | TACI |
| LTB Receptor | RANK | Fn14 | Fas |
| TRAILRI | DR5 | FRANKL | FASL |
| APRIL | LAT1 | RBAT | DPP4 |
| BAFF | BAFFR | TWEAK | TWEAKR |
| TRAIL | LAMP1 | LAMP2 | CLEC7A |
| CD276 | LIGHT | ICOS | ICOSL |
| LT-alpha | LT-Beta | LT alpha/Beta | TNF |

The engineered extracellular vesicles provided herein can be designed to display a POI or fragment thereof, wherein the POI (or fragment thereof) displayed on the extracellular vesicle activates, blocks, induces, suppresses, agonizes, antagonizes, or modulates the function or expression of the target protein.

In some embodiments of any of the aspects, the target protein is a membrane protein. In some embodiments of any of the aspects, the target protein is a type I membrane protein. In certain embodiments, type I membrane protein is selected from the group consisting of CD1a, CD1b, CD1c, CD1d, CD1e, LEU1 (CD5), CD6, CD7, CD10, ITGB2 (CD18), CD19, CR2 (CD21), CD27, CD28, CD34, integrin alpha-IIb (ITA2B, CD41), platelet glycoprotein IX (CD42a), platelet glycoprotein Ib alpha chain (CD42b), platelet glycoprotein Ib beta chain (CD42c), platelet glycoprotein V (CD42d), B7-1 (CD80), B7-2 (CD86), OX40 (CD134), glucocorticoid-induced TNFR-related protein (GITR, CD357), inducible T-cell costimulatory (ICOS, CD278), ICOS ligand (ICOSL, CD275), Herpes virus entry mediator A (HVEM, CD270), B7-H3 (CD276), B and T lymphocyte attenuator (BTLA, D272), CTLA-4 (CD152), killer cell immunoglobulin-like receptor family (KIR family, CD158 family: CD158a-k; KIR2DL1, KIR2DL2, KIR2DL3, KIR3DP1, KIR2DL4, KIR3DL1, KIRDs1, KIR2DL5A, KIR2D15B, KIR2DS5, KIR2DS1, KIR2DS4, KIR2DS2, KIR3DL2), PD-1 (CD279), PD-L1 (CD274), PD-L2 (CD273), T-cell immunoglobulin mucin receptor 1 (TIM-1, CD365), T-cell immunoglobulin mucin receptor 3 (TIM-3, CD366), T-cell immunoglobulin and mucin domain-containing protein 4 (TIM-4), VISTA, sialic acid-binding Ig-like lectin (SIGLEC) 1 (SIGLEC1, CD169), SIGLEC2 (CD22), SIGLEC3 (CD33), SIGLEC5 (CD170), SIGLEC6 (CD328), SIGLEC7 (CD328), SIGLEC8, SIGLEC9 (CD329), SIGLEC10, TIGIT, PVR (CD155), lysosome associated membrane glycoprotein 1 (LAMP1, CD107a), lysosome associated membrane glycoprotein 2 (LAMP2, CD107b), lysosome associated membrane glycoprotein 3 (LAMP3, CD208), PECAM-1 (CD31), STAB-1, NRP2, CEACAM-1 (CD66a), TCR, VTCN1, NCR3LG1, B7-H7 (CD28H), IFNγ receptor 1, IFNγ receptor 2, CD2, CD4, lymphocyte function-associated antigen 3 (LFA-3, CD58), CD8, CD44, CEACAM3 (CD66d), CD96, IGSF2 (CD101), NECTIN1 (HVEC, CD111), NECTIN2 (CD112), NECTIN3 (CD113), DNAX accessory molecule 1 (DNAM-1, CD226), IL2RB (CD122), tyrosine-protein phosphatase no-receptor type substate 1 (SIRPa, CD172a), signal-regulatory protein beta-1 (SIRPB1, CD172b), signal-regulatory protein gamma (SIRPG, CD172g), OX-2 (CD200), OX-2R (CD200R), LAG3 (CD223), LAIR-1 (CD305), NKp30 (CD337), TWEAKR (CD266), CD3d, CD3e, CD3g, ITGAL (CD11a), ITGAM (CD11b), ITGAX (CD11c), ITGAD (CD11d), FCGR3A (CD16a), IL-4 receptor subunit alpha (IL4RA, CD124), IL-2 receptor subunit alpha (IL2RA, CD25), ITGB1 (CD29), CD30, low affinity immunoglobulin gamma Fc region receptor II-a (CD32a), low affinity immunoglobulin gamma Fc region receptor II-b (CD32b), complement receptor type I (CD35), leukosialin (CD43), CD44, receptor-type tyrosine-protein phosphatase C (CD45), membrane cofactor protein (CD46), integrin alpha-1 (CD49a), integrin alpha-2 (CD49b), integrin alpha-3 (CD49c), integrin alpha-4 (CD49d), integrin alpha-5 (CD49e), integrin alpha-6 (CD49f), intercellular adhesion molecule 3 (ICAM-3, CD50), intercellular adhesion molecule 1 (ICAM-1, CD54), ICAM-4 (CD242), integrin alpha V (ITGAV, CD51), integrin beta 3 (ITGB3, CD61), complement decay accelerating factor (CD55), neural adhesion molecule 1 (NCAM-1, CD56), CD62E, CD62L, CD62P, High affinity immunoglobulin gamma Fc receptor I (CD64), macrosialin (CD68), B-cell antigen receptor complex-associated protein alpha chain (CD79a), B-cell antigen receptor complex-associated protein beta chain (CD79b), CD83, leukocyte immunoglobulin-like receptor subfamily A members (CD85G, CD85H, CD85I), leukocyte immunoglobulin-like receptor subfamily B members (CD85A, CD85B, CD85C, CD85D, CD85F, CD85J, CD85K), Immunoglobulin alpha Fc receptor (CD89), CD91, CD93, FAS (CD95), T-cell surface protein tactile (CD96), CD99, semaphoring-D (CD100), immunoglobulin superfamily member 2 (CD101), intercellular adhesion molecule 2 (ICAM-2, CD102), integrin alpha-E (CD103), integrin beta-4 (ITGB4, CD104), endoglin (CD105), vascular cell adhesion protein 1 (VCAM1, CD106), thrombopoietin receptor (CD110), CD114, macrophage colony-stimulating factor 1 receptor (CSF1R, CD115), Granulocyte-macrophage colony-stimulating factor receptor subunit alpha (CSF2RA, CD116), mast/stem cell growth factor receptor Kit (CD117), leukemia inhibitory factor receptor (LIFR, CD118), interferon gamma receptor 1 (CD119), Tumor necrosis factor receptor superfamily member 1A (TNF-R1, CD120a), Tumor necrosis factor receptor superfamily member 1B (TNF-R2, CD120b), Interleukin-1 receptor type 1 (CD121a), Interleukin-1 receptor type 2 (CD121b), Interleukin-2 receptor subunit beta (CD122), Interleukin-3 receptor subunit alpha (IL3RA, CD123), Interleukin-4 receptor subunit alpha (IL4RA, CD124), Interleukin-5 receptor subunit alpha (IL5RA, CD125), Interleukin-6 receptor subunit alpha (IL6RA, CD126), Interleukin-6 receptor subunit beta (IL6ST, CD130), Interleukin-7 receptor subunit alpha (IL7RA, CD127), Interleukin-9 receptor (CD129), Cytokine receptor common subunit beta (CD131), Cytokine receptor common subunit gamma (CD132), CD135, macrophage stimulating protein receptor (CD136), syndecan-1 (CD138), Platelet-derived growth factor receptor alpha (PDGFRA, CD140a), Platelet-derived growth factor receptor beta (PDGFRB, CD140b), thrombomodulin (CD141), CD142, angiotensin converting enzyme (ACE, CD143), cadherin-5 (CD144), melanoma and adhesion molecule (MCAM, CD146), basigin (BSG, CD147), CD148, Signaling lymphocytic activation molecule (SLAM, CD150), SLAM family member 4 (SLAMF4, CD244), signaling lymphocytic activation molecule (SLAM) family member 5 (SLAM5, CD84), SLAM family member 6 (SLAMF6, CD352), SLAM family member 7 (SLAMF7, CD319), SLAM family member 8 (SLAMF8, CD353), SLAM family member 9 (SLAM9), Disintegrin and metalloproteinase domain-containing protein 8 (ADAM8, CD156a), Disintegrin and metalloproteinase domain-containing protein 17 (ADAM17, CD156b), Disintegrin and metalloproteinase domain-containing protein 10 (ADAM10, CD156c), P-selectin glycoprotein 1 (SELPLG, CD162), CD163, CD164, activated leukocyte cell adhesion molecule (ALCAM, CD166), epithelial discoidin domain containing receptor 1 (CD167a), discoidin domain containing receptor 2 (CD167b), neural cell adhesion molecule L1 (L1CAM, CD 171), CD180, endothelial protein C receptor (EPCR, CD201), angiopoietin-1 receptor (CD202b), lymphocyte antigen 75 (CD205), macrophage mannose receptor 1 (CD206), IL-10 receptor subunit alpha (IL10RA, CD210), IL-10 receptor subunit beta (IL10RB, CDw210b), IL-12 receptor subunit beta-1 (IL12RB1, CD212), IL-13 receptor subunit alpha-1 (CD213a1), IL-13 receptor subunit alpha-2 (CD213a2), IL-15 receptor subunit alpha (CD215), IL-17 receptor A (CD217), IL-18 receptor 1 (CD218a), IL-18 receptor accessory protein (CD218b), insulin receptor (CD220), insulin-like growth factor 1 receptor (CD221), cation-independent mannos-6phosphate receptor (CD222), mucin-1 (CD227), T-lymphocyte surface antigen Ly-9 (CD229), plexin-C1 (VESPR, CD232), glycophorin-A (CD235a), glycophorin-B (CD235b), basal cell adhesion molecule (CD239), CD246, T-cell surface glycoprotein CD3 zeta chain (CD247), endosialin (CD248), death receptor 3 (DR3, TNFRS25), death receptor 4 (DR4, CD261), death receptor 5 (DR5, CD262), decoy receptor 2 (DcR2, CD264), receptor activator of nuclear factor kappa-B (RANK, CD265), CD271, C-type mannose receptor 2 (CD280), Toll like receptor 1 (CD281), Toll like receptor 2 (CD282), Toll like receptor 3 (CD283), Toll like receptor 4 (CD284), Toll like receptor 6 (CD286), Toll like receptor 8 (CD288), Toll like receptor 9 (CD289), Toll like receptor 10 (CD290), bone morphogenic protein receptor type 1A (CD292), bone morphogenic protein receptor type ID (CwD293), leptin receptor (CD295), CD300a, CD300c, CD302, Neuropilin-1 (CD304), leukocyte-associated immunoglobulin-like receptor 1 (LIAR1, CD305), Fc receptor-like protein 1 (FcRL1, CD307a), Fc receptor-like protein 2 (FcRL2, CD307b), Fc receptor-like protein 3 (FcRL3, CD307c), Fc receptor-like protein 4 (FcRL4, CD307d), Fc receptor-like protein 5 (FcRL5, CD307e), vascular endothelial growth factor receptor 2 (VEGFR2, CD309), prostaglandin F2 receptor negative regulator (PTGFRN, CD315), immunoglobulin superfamily member 8 (IGSF8, CD316), CD320, platelet F11 receptor (F11R, CD321), junctional adhesion molecule B (JAM-B, CD322), cadherin-1 (CD324), cadherin-2 (CD325), epithelial cell adhesion molecule (CD326), fibroblast growth factor 1 (FGFR1, CD331), fibroblast growth factor 2 (FGFR2, CD332), fibroblast growth factor 3 (FGFR3, CD333), fibroblast growth factor 4 (FGFR4, CD334), natural cytotoxicity triggering receptor 1 (NCR1, CD335), natural cytotoxicity triggering receptor 2 (NCR2, CD336), natural cytotoxicity triggering receptor 3 (NCR3, CD337), triggering receptor expressing on myeloid cells 1 (TREM1, CD354), cytotoxic and regulatory T-cell molecule (CRTAM, CD355), tumor necrosis factor receptor superfamily member 21 (CD358), interleukin-21 receptor (IL21R, CD360), protein EVI2B (CD361), syndecan-2 (CD362), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), V-set and immunoglobulin domain-containing protein 1 (VSIG1), V-set and immunoglobulin domain-containing protein 3 (VSIG3), V-set and immunoglobulin domain-containing protein 4 (VSIG4), V-set and immunoglobulin domain-containing protein 8 (VSIG8), butyrophilin subfamily 3 member A1 (BTN3A1, CD277), butyrophilin subfamily 3 member A2 (BTN3A2), butyrophilin subfamily 2 member A1 (BTN2A1), butyrophilin like protein 8 (BTNL8), butyrophilin subfamily 1 member A1 (BTN1A1), isoforms thereof, fragments thereof, and combinations thereof.

In some embodiments of any of the aspects, the target protein is a membrane protein. In some embodiments of any of the aspects, the target protein is a type II membrane protein. In certain embodiments, the type II membrane protein is selected from the group consisting of CD27L (CD70), CD40, CD40L (CD154), 4-1BB (CD137), 4-1BBL (CD137L), OX40L (CD252), glucocorticoid-induced TNF-related ligand (GITRL), LIGHT (CD258), TNF-related apoptosis inducing factor (TRAIL, CD253), CLEC7A (CD369), CD30L (CD153), TL1 (TNFSF15), FasL (CD178), NKG2 family ligands (NKG2A, B, C, D, E, F and H), B cell activating factor (BAFF, CD257), TNF-related weak inducer of apoptosis (TWEAK), RBAT (SLC3A1), ATP1B2, CD94, neprilysin (CD10), CD13, BLAST-2 (CD23), Dipeptidyl peptidase 4 (DPP4, ADCP2, CD26), CD38, CLEC2C (CD69), Transferrin receptor protein 1 (CD71), B-cell differentiation antigen Lyb-2 (CD72), HLA class II histocompatibility antigen gamma chain (CD74), CD75, CD77, natural killer cell antigen KLRD1 (CD94), NKG2-A/B-activating NK receptor (CD159a), NKG2-C (CD159c), killer cell lectin-like receptor subfamily B member 1 (CD161), galactoside alpha-(1,2)-fucosyltransferase 1 (FUT1, CD174), 3-galactosyl-N-acetylglucosamide 4-alpha-L-fucosyltransferase (FUT3), ectonucleotide pyrophosphatase/phosphodiesterase (ENPP) family member 1 (ENPP1, CD203a), ENPP family member 3 (ENPP3, CD203c), macrophage scavenger receptor types I and II (CD204), C-type lectin domain family 4 member K (langerin, CD207), dendritic cell-specific ICAM-3-grabbing non-integrin 1 (DC-SIGN, CD209), CD224, CD238, glutamyl aminopeptidase (CD249), receptor activator of nuclear factor kappa-B ligand (RANKL, CD254), CD298, DC-SIGN related protein (DC-SIGNR, CD299), C-type lectin domain family 10 member A (CLEC10A, CD301), C-type lectin domain family 4 member C (CLEC4C, CD303), NKG2-D type II integral membrane protein (KLRK1, CD314), bone marrow stromal antigen 2 (BST2, CD317), transmembrane and associated with src kinases (TRASK, CD318), protein jagged-1 (CD339), human epidermal growth factor 2 (HER2, CD340), C-type lectin domain family 4 member A (CLEC4A, CD367), C-type lectin domain family 4 member D (CLEC4D, CD368), C-type lectin domain family 7 member A (CLEC7A, CD369), C-type lectin domain family 9 member A (CLEC9A, CD370), C-type lectin domain family 12 member A (CLEC12A, CD371), SLC3A2 (CD98 heavy chain), tumor necrosis factor (TNF, TNF-alpha, TNFα), lymphotoxin-alpha (LTA, LT-α) also known as tumor necrosis factor ligand superfamily member 1 (TNF-beta, TNF-β), tumor necrosis family ligand superfamily member 3 also known as lymphotoxin beta (LTB, TNF-C, TNFγ), tumor necrosis factor ligand superfamily 15 (TL1A), isoforms thereof, fragments thereof, and combinations thereof.

In some embodiments of any of the aspects, the target protein is a membrane protein. In some embodiments of any of the aspects, the target protein is a type III membrane protein. In certain embodiments, the type II membrane protein is selected from the group consisting of B cell activating factor (BAFFR, CD268), glycophorin-C(CD236), transmembrane activator and CAML interactor (TACI, CD267), B-cell maturation protein (BCM, CD269), or any isoform, fragment thereof, or the like known by one of ordinary skill in the art.

In some embodiments of any of the aspects, the target protein is a membrane protein. In some embodiments of any of the aspects, the target protein is a multi-pass membrane protein. In certain embodiments, the multi-pass membrane protein is selected from the group consisting of Alpha-2A adrenergic receptor (A2AR, ADRA2A), adenosine receptor A2b (A2BR, ADORA2B), NOX2, SLC7A5 (CD98 light chain), CD39, CD47, PVRIG (CD112R), CD9, CD20, CD36, CD37, CD53, CD63, CD81, CD82, C5a receptor (CD88), CD92, CD97, prominin-1 (CD133), CD151, high affinity interleukin-8 receptor A (IL8RA, CXCR1, CD181), high affinity interleukin-8 receptor B (IL8RB, CXCR2, CD182), C—X—C chemokine receptor (CXCR) type 3 (CXCR3, CD183), CXCR4 (CD184), CXCR5 (CD185), CXCR6 (CD186), C—C chemokine (CCR) type 1 (CCR1, CD191), CCR2 (CD192), CCR3 (CD193), CCR4 (CD194), CCR5 (CD195), CCR6 (CD196), CCR7 (CD197), CCR8 (CDw189), CCR9 (CDw199), CD231, solute carrier family 4 member 1 (SLC4A1, CD233), Duffy antigen/chemokine receptor (DARC, CD234), blood group Rh (CE) polypeptide (CD240CE), blood group Rh (D) polypeptide (CD240D), ammonium transporter Rh type A (CD241), CD243, calcium signal-modulating cyclophilin ligand (CAMLG), prostaglandin D2 receptor 2 (PTGDR2, CD294), EGF-like module receptor 2 (CD312), CD338, frizzled-4 (CD344), frizzled-9 (CD349), frizzled-10 (CD350), sphingosine 1-phosphate receptor 1 (CD363), isoforms thereof, fragments thereof, and combinations thereof.

In some embodiments of any of the aspects, the target protein is a membrane protein. In some embodiments of any of the aspects, the target protein is lipid anchored membrane protein. In some embodiments of any of the aspects, the target protein is a GPI linked membrane protein. In certain embodiments, the GPI linked membrane protein is selected from the group consisting of CD160, RGMB, CEACAM8 (CD66b, CD67), CEACAM6 (CD66c), CEACAM5 (CD66e), CD73, CD14, FCGR3B (CD16b), CD24, BLAST-1 (CD48), CAMPATH-1 (CD52), CD59, CD87, CD90, semaphorin-7A (CD108), CD109, bone marrow stromal cell antigen 1 (BSTI, CD157), CD177, melanotransferrin (CD228), CD230, decoy receptor 1 (DcR1, CD263), CD296, CD297, isoforms thereof, fragments thereof, and combinations thereof.

In some embodiments of any of the aspects, the target protein is a secreted protein. In certain embodiments, the secreted protein is a cytokine. In certain embodiments, the secreted protein is a chemokine. In certain embodiments, the secreted protein is an interferon. In certain embodiments, the secreted protein is an interferon selected from the group consisting of IFNA1, IFNA2, IFNA4, IFNA5, IFA6, IFNA7, IFNA8, IFNA10, IFNA13, IFNA14, IFNA16, IFNA17, IFNA21, IFNB1, isoforms thereof, fragments thereof, and combinations thereof.

In some embodiments of any of the aspects, the target protein is a secreted protein. In certain embodiments, the secreted protein is a lymphokine. In certain embodiments, the secreted protein is a member of the tumor necrosis factor super family. In certain embodiments, the secreted protein is a secreted isoform of a member of the tumor necrosis factor super family. In certain embodiments, the secreted tumor necrosis factor super family member is selected from the group consisting of lymphotoxin alpha (LTA), lymphotoxin beta (LTB), tumor necrosis factor (TNF), CD40L FASLG (CD178), TNF-related apoptosis induced ligand (CD253), CD254, TNF-related weak inducer of apoptosis (TWEAK), A proliferation inducing ligand (APRIL, CD256), B cell activating factor (BAFF, CD257), LIGHT (CD258), TNF ligand-related molecule 1 (TL1), ectodysplasin-A (EDA), isoforms thereof, fragments thereof, and combinations thereof.

In some embodiments of any of the aspects, the target protein is a secreted protein. In certain embodiments, the secreted protein is an interleukin. In certain embodiments, the interleukin target protein is selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-34, IL-35, IL-36, IL-37, IL-38, IL-39, and IL-40. In certain embodiments, the secreted protein is selected from the group consisting of fibrinogen-like protein 1 (FGL-1), TSG-6, NRP1, SEMA3A, SEMA3F, IFNγ, WNT5A, PSG1 (CD66f), collagen family of proteins (for example but not limited to collagen I, collagen II, collagen III alpha 1, collagen IV, collagen XXIII alpha 1, collagen XXV alpha 1), pregnancy-specific beta-1-glycoprotein 1 (PSG1, CD66f), leukocyte-associated immunoglobulin-like receptor 2 (LIAR2, CD306), peptidase inhibitor 16 (CD364), netrin-1 (NET-1), colony-stimulating factor (CSF), decoy receptor 3 (DcR3, TNFRSF6B), isoforms thereof, fragments thereof, and combinations thereof.

In some embodiments of any of the aspects, the target protein is a T cell engager or the like known by one of ordinary skill in the art, for example a bispecific T cell engager (such as an artificial bispecific monoclonal antibody). In some embodiments of any of the aspects, the target protein is a chimeric antigen receptor (CAR), or the like known by one of ordinary skill in the art. In some embodiments of any of the aspects, the target protein is on a CAR-T cell. In some embodiments of any of the aspects, the target protein is on a CAR-B cell. In some embodiments of any of the aspects, the target protein is on a CAR-Natural Killer cell. In some embodiments of any of the aspects, the target protein is on a CAR-dendritic cell. In some embodiments of any of the aspects, the target protein is on a CAR-macrophage cell. In some embodiments of any of the aspects, the target protein is on a CAR-regulatory T cell. In some embodiments of any of the aspects, the target protein is on a CAR-gamma delta T cell. In some embodiments of any of the aspects, the target protein is on a CAR mucosal-associated invariant T cell. In some embodiments of any of the aspects, the target protein is on a CAR-tissue-resident memory T cell.

### Linkers

In some embodiments of any of the aspects provided herein, the fusion polypeptide further comprises a peptide linker. The linker may be flexible, rigid, or cleavable. Further, the linker can be linked directly or via another linker (e.g., a peptide of one, two, three, four, five, six, seven, eight, nine, ten or more amino acids) to the fusion polypeptides described herein. Linkers can be configured according to a specific need, e.g., based on at least one of the following characteristics. In some embodiments of any of the aspects, linkers can be configured to have a sufficient length and flexibility such that it can allow for a cleavage at a target site, for example proteolytic cleavage at a proteolytic site. In some embodiments of any of the aspects, linkers can be configured to allow multimerization of the fusion polypeptides provided herein, for example two fusion polypeptides multimerizing to form a dimer. In some embodiments of any of the aspects, linkers can be configured to allow multimerization of one or more fusion polypeptides provided herein, for example two or more fusion polypeptides multimerize to form a multimer, wherein the multimerization of the two or more fusion polypeptides is mediated by covalent or noncovalent intermolecular forces between linkers of the said two or more fusion polypeptides. In some embodiments of any of the aspects, linkers can be configured to facilitate expression and purification of the fusion polypeptides or engineered extracellular vesicles provided herein.

In some embodiments of any of the aspects, a linker can be configured to have any length in a form of a peptide, peptidomimetic, an aptamer, a protein, a nucleic acid (e.g., DNA or RNA), or any combinations thereof. In some embodiments of any of the aspects, a linker can be an amino acid. For example, the linker may be a glycine (Gly) residue. In another embodiment, the linker may be a polypeptide linker such as Gly-Ser-Ser-Gly (GSSG) (SEQ ID NO: 154). In another embodiment, the linker may be a polypeptide linker such as Gly-Gly-Gly-Gly-Ser (GGGGS) (SEQ ID NO: 156). In some embodiment of the fusion polypeptide presented herein, there may be multiple linkers. In some embodiment of the fusion polypeptide presented herein, there may be a plurality of various linkers. For example, a fusion polypeptide may have one, two, three, four, five, six, seven, eight, nine, ten, eleven, twelve, thirteen, fourteen, fifteen, sixteen, seventeen, eighteen, nineteen, twenty, twenty-one, or more linkers. In some embodiments the one or more linkers may be directly adjacent to one another and connected via a covalent peptide bond. In some embodiments of the fusion polypeptide there are multiple consecutive Gly linkers, herein labeled (G) n, where n denotes a positive number of equal to or greater than one (e.g., 1, 2, 3, 4, 5 etc.). In some embodiments of the fusion polypeptide, there are multiple consecutive GSSG (SEQ ID NO: 154) linkers, herein labeled (GSSG) n, where n denotes a positive number of equal to or greater than one (e.g., 1, 2, 3, 4, 5, etc.). In some embodiments of the fusion polypeptide, there are multiple consecutive GGGGS linkers, herein labeled (GGGGS) n, where n denotes a positive number of equal to or greater than one (e.g., 1, 2, 3, 4, 5 etc.). In some embodiments of the fusion polypeptide, there may be multiple G, GSSG (SEQ ID NO: 154), and GGGGS linkers arranged in any combination and number. One of skill in the art can appreciate that a fusion polypeptide can include one or more combinations of different linkers, or that one can use a plurality of linkers, each including one or more different linker sequences.

In another embodiment the linker may be a protein sequence for a self-cleavable peptide. For example, 2A sequences such as P2A, E2A, F2A, and T2A code for self-cleavable peptides by inducing ribosomal slippage on the mRNA at the 2A site which prevents peptide bond formation. The slippage will result in two separate peptides after translation. This allows the expression of two separate proteins from one promoter region. In some embodiments of any of the aspects, the linker is a P2A peptide linker. P2A is a self-cleaving peptide sequence allowing for expression of two proteins from one promoter. In some embodiments, the P2A linker is encoded by the nucleic acid sequence: GCTACTAACTTCAGCCTGCTGAAGCAG (SEQ ID NO: 172). The amino acid sequence of P2A is ATNFSLKQAGDVENPGP (SEQ ID NO: 173). Any combination of the proteins described herein may be expressed with a self-cleavable peptide as known by one of ordinary skill in the art.

In some embodiments of any of the aspects, the polypeptide linker is a non-cleavable linker. In some embodiments of any of the aspects, a linker can be a chemical linker of any length.

In some embodiments of any of the aspects, the polypeptide linker is an Fc linker, for example an Fc from an immunoglobulin. An exemplary nucleic acid sequence encoding an Fc polypeptide is SEQ ID NO: 143 or a fragment thereof. An exemplary amino acid sequence of an Fc polypeptide is SEQ ID NO: 144. In some embodiments of any of the aspects, the linker is an Fc mutein linker. An exemplary nucleic acid sequence encoding an Fc mutein polypeptide is SEQ ID NO: 145. An exemplary amino acid sequence of an Fc mutein polypeptide is SEQ ID NO: 146.

In some embodiments of any of the aspects, the linker provides a multimerization (e.g., dimerization) domain, wherein one fusion polypeptide may connect with another fusion polypeptide at each fusion polypeptide's respective multimerization domain. Specific amino acid sequences or multimerization domains of the fusion polypeptides facilitate multimerization of the fusion polypeptides. Multimerization of multiple fusion polypeptides will provide multiple fusion polypeptides within close proximity to one another to one or more a target receptor on the target cell, wherein the multiple fusion peptides will enhance receptor clustering on the target cell. For example, increasing density of agonist presentation supports a receptor clustering on a target cell—an onerous barrier for traditional receptor agonism targeting strategies. Clustering receptors on a target cell will result in enhanced signal transduction. Without receptor clustering a signal may be weaker or not occur all together. For example, Fc domain sequences presented herein dimerize resulting in two fusion polypeptides connected by a covalent bond via the two Fc domains on their respective fusion polypeptide. One preferred embodiment of an Fc domain is from IgG4, herein labeled 4Fc (nucleic acid sequence SEQ ID NO: 151 and amino acid sequence SEQ ID NO: 152). In other embodiments Fc may be from IgG heavy chain (IgG H) (nucleic acid sequence SEQ ID NO: 141 and amino acid sequence SEQ ID NO: 142). In certain embodiments Fc from another immunoglobulin known in the art, (e.g., IgG2, IgG3, etc.) may be used. In some embodiments, the nucleic acid sequence of IgG2 is SEQ ID NO: 147. In some embodiments, the amino acid sequence of IgG2 is SEQ ID NO: 148. In some embodiments, the nucleic acid sequence of IgG3 is SEQ ID NO: 149. In some embodiments, the amino acid sequence of IgG3 is SEQ ID NO: 150.

In certain embodiments, the Fc comprises amino acid substitutions, deletions, or insertions that modulate Fc function. In various embodiments, the Fc mutein has reduced or abolished Fc-mediated effector functions, for example, reduced or abolished Fc-Fc Receptor (FcR) mediated effector functions. In various embodiments, the FcR mutein has reduced or abolished FcγRI binding. In certain embodiments, the Fc comprises amino acid substitutions, deletions, or insertions that reduce, abolish, or abrogate binding of said Fc to Fc receptors (e.g., FcγR, FcR). In certain embodiments, the Fc comprises amino acid substitutions, deletions, or insertions that abrogate binding of said Fc to Fc receptors (e.g., FcγR, FcR), abolishing antibody directed cytotoxicity (ADCC) effector function. Fc comprising amino acid substitutions, deletions, or insertions that abrogate binding of said Fc to Fc receptors (e.g., FcγR, FcR), abolishing ADCC effector function are herein labelled Fc mutein. In certain embodiments the Fc mutein is an IgG1 Fc mutein. In certain embodiments, the IgG1 Fc mutein comprises amino acid substitutions, deletions, or insertions that abrogate binding of Fc receptors (e.g., FcγR, FcR), abolishing antibody directed cytotoxicity (ADCC) effector function. In certain embodiments, the IgG1 Fc mutein comprises amino acid substitutions L234A, L235A, P329G (SEQ ID NO: 146) that abrogate binding of Fc receptors (e.g., FcγR, FcR), abolishing antibody directed cytotoxicity (ADCC) effector function. In certain embodiments, the IgG2 Fc mutein comprises amino acid that abrogate binding of Fc receptors (e.g., FcγR, FcR), abolishing ADCC effector function. In certain embodiments, the IgG3 Fc mutein comprises amino acid that abrogate binding of Fc receptors (e.g., FcγR, FcR), abolishing ADCC effector function. In certain embodiments, the IgG4 Fc mutein comprises amino acid that abrogate binding of Fc receptors (e.g., FcγR, FcR), abolishing ADCC effector function.

In some embodiments, the Fc mutein may be a Knob-into-Hole (KiH) Fc mutein. KiH Fc muteins refers to an Fc mutein wherein specific amino acid residues are mutated to enhance the interaction interface or stability of interaction or structural integrity of interactions between two Fc domains. The knob residue on one Fc chain fits into a complimentary hole in another Fc chain, resulting in enhanced interface interaction or stability of interaction or structural integrity of interactions between two Fc domains. KiH Fc muteins can be used to homodimerize two fusion polypeptides. KiH Fc muteins can be used to heterodimerize two fusion polypeptides. KiH Fc muteins can be used to form homodimers of two the same fusion polypeptides. KiH Fc muteins can be used to form heterodimers of two different fusion polypeptides. KiH Fc muteins can be used to homodimerize two fusion polypeptides. KiH Fc muteins can be used to heterodimerize two fusion polypeptides. KiH Fc muteins can be used to form homodimers of two of the same fusion polypeptides. KiH Fc muteins can be used to form heterodimers of two different fusion polypeptides.

Non-limiting examples of KiH muteins are T366W knob pairing with the hole mutations T336S, L368A, Y407V in Fc CH3 domains of Fc. The KiH Fc muteins T366W (knob) and T336S, L368A, Y407V (hole) can be used for Fc-Fc dimerization of two individual fusion polypeptides. A fusion polypeptide comprising a linker with an Fc mutein further comprising a T366W mutation may heterodimerize with a fusion polypeptide comprising a linker with an Fc mutein further comprising one or more hole (e.g., T336S, L368A, Y407) mutations. Another non-limiting example of Knob-into-Hole muteins are cysteine mutations S354C knob pairing with Y349C hole in CH3 domains of Fc (Vaks, L.; Litvak-Greenfeld, D.; Dror, S.; Shefet-Carasso, L.; Matatov, G.; Nahary, L.; Shapira, S.; Hakim, R.; Alroy, I.; Benhar, I. Design Principles for Bispecific IgGs, Opportunities and Pitfalls of Artificial Disulfide Bonds. Antibodies 2018, 7, 27. doi.org/10.3390/antib7030027). The KiH Fc muteins S354C knob and Y349C hole can be used for Fc-Fc dimerization of two individual fusion polypeptides. A fusion polypeptide comprising a linker with an Fc mutein further comprising a S354C mutation may heterodimerize with a fusion polypeptide comprising a linker with an Fc mutein further comprising Y349C hole mutation.

The KiH Fc muteins presented herein can be used to homodimerize two fusion polypeptides. The KiH Fc muteins presented herein can be used to heterodimerize two fusion polypeptides. The KiH Fc muteins presented herein can be used to form homodimers of two the same fusion polypeptides. The KiH Fc muteins presented herein can be used to form heterodimers of two different fusion polypeptides. The KiH Fc muteins presented herein can be used to homodimerize two fusion polypeptides. The KiH Fc muteins presented herein can be used to heterodimerize two fusion polypeptides. The KiH Fc muteins presented herein can be used to form homodimers of two of the same fusion polypeptides. The KiH Fc muteins presented herein can be used to form heterodimers of two different fusion polypeptides.

In some embodiments of any of the aspects, the linker provides a multimerization domain wherein one fusion polypeptide may connect with at least one other fusion polypeptide at each fusion polypeptide's respective multimerization domain. Multimerization of multiple fusion polypeptides will provide multiple fusion polypeptides within close proximity to one another to one or more a target receptor on the target cell, wherein the multiple fusion peptides will enhance receptor clustering on the target cell. Clustering receptors on a target cell will result in enhanced signal transduction. Without receptor clustering a signal may be weaker or not occur all together. The multimerization domains may form heterotypic or homotypic multimers. The multimerization domains may multimerize in either a heterotypic or homotypic fashion. The interaction between multiple individual fusion polypeptides may be facilitated by the multimerization of the multimerization domain present on each respective fusion polypeptide.

In some embodiments of any of the aspects, the linker provides a dimerization domain wherein one fusion polypeptide may connect with at least one other fusion polypeptide at each fusion polypeptide's respective dimerization domain. In some embodiments of any of the aspects, the linker provides a dimerization domain wherein one fusion polypeptide may connect with at least one other fusion polypeptide at each fusion polypeptide's respective dimerization domain. Dimerization of multiple fusion polypeptides will provide multiple fusion polypeptides within close proximity to one another to one or more a target receptor on the target cell, wherein the multiple fusion peptides will enhance receptor clustering on the target cell. Clustering receptors on a target cell will result in enhanced signal transduction. Without receptor clustering a signal may be weaker or not occur all together. The dimerization domains may form heterodimers or homodimers. The dimerization domains can dimerize in either a heterotypic or homotypic fashion. The interaction between two fusion polypeptides may be facilitated by the homodimerization of the dimerization domain present on each respective fusion polypeptide.

A non-limiting example of a dimerization domain that can be used to dimerize two fusion polypeptides is a PDZ domain. PDZ domains presented herein may dimerize resulting in two fusion polypeptides connected via the two PDZ domains on their respective fusion polypeptide. Non-limiting examples of PDZ proteins in humans are AAG12, AHNAK, AHNAK2, AIP1, ALP, APBA1, APBA2, APBA3, ARHGAP21, ARHGAP23, ARHGEF11, ARHGEF12, CARD10, CARD11, CARD14, CASK, CLP-36, CNKSR2, CNKSR3, CRTAM, DFNB31, DLG1, DLG2, DLG3, DLG4, DLG5, DVL1, DVL1L1, DVL2, DVL3, ERBB2IP, FRMPD1, FRMPD2, FRMPD2L1, FRMPD3, FRMPD4, GIPC1, GIPC2, GIPC3, GOPC, GRASP, GRIP1, GRIP2, HTRA1, HTRA2, HTRA3, HTRA4, IL16, INADL, KIAA1849, LDB3, LIMK1, LIMK2, LIN7A, LIN7B, LIN7C, LM07, LNX1, LNX2, LRRC7, MAGI1, MAGI2, MAGI3, MAGIX, MAST1, MAST2, MAST3, MAST4, MCSP, MLLT4, MPDZ, MPP1, MPP2, MPP3, MPP4, MPP5, MPP6, MPP7, MYO18A, NHERF1, NOS1, PARD3, PARD6A, PARD6B, PARD6G, PDLIM1, PDLIM2, PDLIM3, PDLIM4, PDLIM5, PDLIM7, PDZD11, PDZD2, PDZD3, PDZD4, PDZD5A, PDZ6, PDZD7, PDZD8, PDZK1, PDZRN3, PDZRN4, PICK1, PPP1R9A, PPP1R9B, PREX1, SDCBP2, SHANK1, CHANK2, SHANK3, SHROOM2, SHROOM3, SHROOM4, SIPA1, SIPA1L1, SIPA1L2, SIPA1L3, SLC9A3R1, SLC9A3R2, SNTA1, SNTB1, SNTB2, SNTG1, SNTG2, SNX27, SPLA2, STXBP4, SYNJ2BP, SYNPO2, SYNPO2L, TAX1BP3, TIAM1, TIAM2, TJP1, TJP2, TJP3, TRPC4, TRPC5, USH1C, and WHRN.

PDZ domains presented herein may form heterodimers or homodimers. The PDZ domains can dimerize in either a heterotypic or homotypic fashion. The interaction between two fusion polypeptides may be facilitated by the homodimerization of the PDZ domain present on each respective fusion polypeptide. For example, SHANK1 PDZ domains form PDZ-PDZ homodimers (Im Y J, Lee J H, Park S H, Park S J, Rho S H, Kang G B, Kim E, Eom S H. Crystal structure of the Shank PDZ-ligand complex reveals a class I PDZ interaction and a novel PDZ-PDZ dimerization. J Biol Chem. 2003 Nov. 28; 278 (48): 48099-104. doi: 10.1074/jbc.M306919200. Epub 2003 Sep. 3. PMID: 12954649). Other examples of PDZ-PDZ homodimers are GRIP1-GRIP1 and GRIP2-GRIP2.

The interaction between two fusion polypeptides may be facilitated by the heterodimerization of the PDZ domain present on each respective fusion polypeptide. For example, NOS1 and GRIP1 heterodimerize with SNTA1 and PDZ6, respectively (Chang B H, Gujral T S, Karp E S, BuKhalid R, Grantcharova V P, MacBeath G. A systematic family-wide investigation reveals that ˜30% of mammalian PDZ domains engage in PDZ-PDZ interactions. Chem Biol. 2011 Sep. 23; 18 (9): 1143-52. doi: 10.1016/j.chembiol.2011.06.013. PMID: 21944753; PMCID: PMC3442787). The PDZ dimerization domains presented herein can be used to homodimerize two fusion polypeptides. The PDZ dimerization domains presented herein can be used to heterodimerize two fusion polypeptides. PDZ domains can be used to form homodimers of two the same fusion polypeptides. PDZ domains can be used to form heterodimers of two different fusion polypeptides. The PDZ domains of the PDZ domain containing proteins presented herein can be used to homodimerize two fusion polypeptides. The PDZ domains of the PDZ domain containing proteins presented herein can be used to heterodimerize two fusion polypeptides. The PDZ domains of the PDZ domain containing proteins presented herein can be used to form homodimers of two of the same fusion polypeptides. The PDZ domains of the PDZ domain containing proteins presented herein can be used to form heterodimers of two different fusion polypeptides.

Another non-limiting example of a dimerization domain or motif that can be used to dimerize two fusion polypeptides is a coiled-coil domain. For example, the coiled-coil domain from myosin that plays a role in myosin subunit association. A non-limiting example of a coiled-coil dimerization domain that can be used to dimerize two fusion polypeptides is a basic Leucine Zipper Domain (bZIP) domain. The leucine rich pattern in bZIP domains from coiled-coil structural motifs that enable protein-protein interactions and facilitate dimerization. Non-limiting examples of bZIP domain containing proteins are c-Fos, c-Jun, Activating Transcription Factor (ATF) Family (e.g., ATF1, ATF2, ATF3, ATF4, ATF5, and ATF6), cAMP Response Element-Binding (CREB) Family (e.g., CREB1, CREM, ATF1, ATF2, and ATF4), Nuclear Factor erythroid 2-related factor (Nrf) Family (e.g., Nrf1 (NFE2L1), Nrf2 (NFE2L2), and Nrf3 (NFE2L3)), X-box binding protein 1 (XBP1), General Control Nondepressible 4 (GCN4), c-Myc-interacting zinc finger protein (ZIP) Family (e.g., ZIP1, ZIP2, ZIP3, ZIP4), CCAAT/enhancer-binding protein (CEBP) Family (e.g., CEBP CEBPα, CEBPβ, CEBPγ, CEBPδ, CEBPε, and CEBPζ) and Activating Transcription Factor 6 (ATF6). Proteins containing bZIP domain may form heterodimers or homodimers. The Coiled-coil domains of the Coiled-coil domain containing proteins presented herein can be used to homodimerize two fusion polypeptides. The Coiled-coil domains of the Coiled-coil domain containing proteins presented herein can be used to heterodimerize two fusion polypeptides. The Coiled-coil domains of the Coiled-coil domain containing proteins presented herein can be used to form homodimers of two of the same fusion polypeptides. The Coiled-coil domains of the Coiled-coil domain containing proteins presented herein can be used to form heterodimers of two different fusion polypeptides.

Another non-limiting example of a dimerization domain that can be used to dimerize two fusion polypeptides is a basic Helix-Loop-Helix (HLH) domain. HLH domains are protein structural motifs that facilitate protein-protein interactions and the formation of homodimers or heterodimers. Non-limiting examples of HLH domain containing proteins are c-Myc, Max, MyoD, E12/E47 (E2A), Neurogenins (e.g., Ngn1, Ngn2, and Ngn3), Inhibitor of DNA binding (Id) proteins, Hypoxia-Inducible Factor 1 Alpha (HIF-la), Transcription Factor E3 (TFE3), Transcription Factor EB (TFEB), and Achaete-Scute Complex (AS-C) proteins. The interaction between two fusion polypeptides may be facilitated by the heterodimerization of the HLH domain present on each respective fusion polypeptide. For example, the HLH Domain of Myc heterodimerizes with the HLH domain of Max. The interaction between two fusion polypeptides may be facilitated by the homodimerization of the HLH domain present on each respective fusion polypeptide. For example, the HLH domain of MyoD, Neurogenins, TFE3, TFEB, and AS-C proteins can homodimerizes. The HLH domain of E2A may homodimerize or heterodimerize with other HLH proteins. The HLH domains of the HLH domain containing proteins presented herein can be used to homodimerize two fusion polypeptides. The HLH domains of the HLH domain containing proteins presented herein can be used to heterodimerize two fusion polypeptides. The HLH domains of the HLH domain containing proteins presented herein can be used to form homodimers of two the same fusion polypeptides The HLH domains of the HLH domain containing proteins presented herein can be used to form heterodimers of two different fusion polypeptides.

Another non-limiting example of dimerization domains that can be used to dimerize two fusion polypeptides are Src Homology 2 (SH2) and Src Homology 3 (SH3) domains of Src kinase and Grb2 adaptor protein, respectively. SH2 domains are structural motifs that mediate protein-protein interactions by binding phosphorylated tyrosine residues on target proteins, for example target proteins displaying the amino acid motif Y-X-X-M where Y represents tyrosine, M represents methionine, and X represents any amino acid. A non-limiting example of an SH2 domain containing proteins is Growth Factor Receptor-Bound Protein 2 (Grb2) that binds to tyrosine residues in receptor tyrosine kinases. SH3 domains are structural motifs that mediate protein-protein interactions by binding proline rich sequences on target proteins, for example target proteins displaying the amino acid motif P-X-X-P where P represents proline and X represents any amino acid. A non-limiting example of an SH3 domain containing proteins is Src Kinase that interacts with proline-rich residues in target proteins. The SH2 dimerization domains presented herein can be used to homodimerize two fusion polypeptides. The SH2 dimerization domains presented herein can be used to heterodimerize two fusion polypeptides. The SH3 dimerization domains presented herein can be used to homodimerize two fusion polypeptides. The SH3 dimerization domains presented herein can be used to heterodimerize two fusion polypeptides. SH2 domains can be used to form homodimers of two of the same fusion polypeptides. SH2 domains can be used to form heterodimers of two different fusion polypeptides. SH3 domains can be used to form homodimers of two of the same fusion polypeptides. SH3 domains can be used to form heterodimers of two different fusion polypeptides.

Another non-limiting example of a dimerization domain that can be used to dimerize two fusion polypeptides is a Sterile Alpha Motif (SAM) domain. SAM domains are structural motifs involved in protein-protein interactions through homotypic or heterotypic interactions or higher-order oligomers. Homotypic and heterotypic interactions refer to intermolecular interactions between the same versus different molecules, respectively. This concept can be extended to distinguish interactions between the same versus different motifs on molecules. Higher-order oligomers, that is, oligomers in which the number of monomers in a complex is broadly distributed and can be large, have important functions in signal transduction and cell fate decisions. Non-limiting examples of SAM domain containing proteins are TEL Transcription Factor (ETV6), Ephrin Receptor-Interacting Protein (EPB4.1L5), Traf2- and Nck-Interacting Kinase (TNIK), and Sterile Alpha and TIR Motif Containing 1 (SARM1) protein. The interaction between two fusion polypeptides may be facilitated by the homodimerization of the SAM domain present on each respective fusion polypeptide. For example, the SAM domain of TEL Transcription Factor (ETV6), Ephrin Receptor-Interacting Protein (EPB4.1L5), Traf2- and Nck-Interacting Kinase (TNIK), and Sterile Alpha and TIR Motif Containing 1 (SARM1) protein mediate formation of homodimers.

The SAM domains of the SAM domain containing proteins presented herein can be used to homodimerize two fusion polypeptides. The SAM domains of the SAM domain containing proteins presented herein can be used to heterodimerize two fusion polypeptides. The SAM domains of the SAM domain containing proteins presented herein can be used to form homodimers of two of the same fusion polypeptides. The SAM domains of the SAM domain containing proteins presented herein can be used to form heterodimers of two different fusion polypeptides.

Another non-limiting example of a dimerization domain that can be used to dimerize two fusion polypeptides is a Toll/Interleukin-1 Receptor (TIR) domain that facilitates homodimerization. TIR domains are characterized by a conserved α/β sandwich fold and present in IL18R1 (CDw218a), IL18RAP (CDw218b), IL1R1 (CD121a), IL1RAP, IL1RAPL1, IL1RAPL2, IL1RL1, IL1RL2, MYD88, SIGIRR (TIR8), TLRs (e.g., TLR1 (CD281), TLR10 (CD290), TLR2 (CD282), TLR3 (CD283), TLR4 (CD284), TLR5 (CD285), TLR6 (CD286), TLR7 (CD287), TLR8 (CD288), TLR9 (CD289) and SARM1 (MyD88-5). TIR domains provide interfaces that allow for the formation of homodimers. TIR domains provide interfaces that allow for the formation of homodimers of two TLRs. The interaction between two fusion polypeptides may be facilitated by the homodimerization of the TIR domain present on each respective fusion polypeptide. For example, the TIR domain of TLR2, TRL3, TLR4, TLR7, TLR8, and TLR9 proteins mediate formation of homodimers. The interaction between two fusion polypeptides may be facilitated by the heterodimerization of the TIR domain present on each respective fusion polypeptide. For example, the TIR domain of TLR1, TLR5, TLR6, and TLR10 proteins mediate formation of heterodimers.

The TIR domains of the TIR domain containing proteins presented herein can be used to homodimerize two fusion polypeptides. The TIR domains of the TIR domain containing proteins presented herein can be used to heterodimerize two fusion polypeptides. The TIR domains of the TIR domain containing proteins presented herein can be used to form homodimers of two of the same fusion polypeptides. The TIR domains of the TIR domain containing proteins presented herein can be used to form heterodimers of two different fusion polypeptides.

Another non-limiting example of a dimerization domain that can be used to dimerize two fusion polypeptides is a RING domain. RING domains can be used to form homodimers or heterodimers of two fusion polypeptides. Ring domains are typically 40-60 amino acids in length and characterized by a zinc-binding fold and primarily serve as a scaffold for ubiquitin ligase complexes, for example E2 and E3 ligases. Individual RING domains provide interfaces that allow for the formation of dimers between two separate RING domains. For example, the RING domains between two ubiquitin-conjugating (E2) enzymes homodimerize facilitating activation of the E2 enzyme to transfer ubiquitin from one E2 to a substrate protein. In another example, the RING domain in Casitas B-lineage Lymphoma (CBL) protein is essential for homodimerization of CBL leading to enhanced activation of CBL protein's E3 ubiquitin ligase activity and regulation of protein degradation. In another example, homodimerization of the RING domain of individual MDM2 proteins homodimerizes two MDM2 proteins leading to enhancement of MDM2 ubiquitin ligase activity. A non-limiting example of a RING heterodimer is the heterodimerization of the RING domains from BRCA1 and BARD1 wherein the RING domain of BRCA1 heterodimerizes with the RING domain of BARD1 leading to E3 ubiquitin ligase activity. Another non-limiting example of a RING heterodimer is the heterodimerization of the RING domains from RING1A1 and BMI1 wherein the RING domain of RING1A1 heterodimerizes with the RING domain of BMI1 leading to ubiquitin ligase activity of PRC1. The RING dimerization domains presented herein can be used to homodimerize two fusion polypeptides. The RING dimerization domains presented herein can be used to heterodimerize two fusion polypeptides. RING domains can be used to form homodimers of two of the same fusion polypeptides. RING domains can be used to form heterodimers of two different fusion polypeptides.

Another non-limiting example of a dimerization domain that can be used to dimerize two fusion polypeptides is subdomain IIA of human serum albumin (HSA) domain. The HSA protein is primarily known to exist in a monomeric state, however, under certain conditions HSA dimerizes or multimerizes into higher-order oligomers. Conditions that influence dimerization or multimerization of HSA subdomain IIA monomers include heat, pressure, pH, and ionic strength wherein under favorable conditions hydrophobic patches facilitate protein-protein interactions and stabilization of HAS dimers or oligomers. Subdomain IIA of HSA dimerization domains presented herein can be used to homodimerize two fusion polypeptides. Subdomain IIA of HSA domain presented herein can be used to multimerize more than two fusion polypeptides. HSA domains can be used to form homodimers of two of the same fusion polypeptides. HSA domains can be used to form heterodimers of two different fusion polypeptides.

Another non-limiting example of a dimerization domain that can be used to dimerize two fusion polypeptides is the Regulator of G Protein Signaling (RGS) domain of G protein coupled receptors. RGS domains can be used to form homodimers or heterodimers of two fusion polypeptides. RGS domains are approximately 120 amino acids in length and adopt a conserved alpha-helical fold, and primarily serve as regulators of G protein signaling. RGS domains facilitate dimerization wherein the individual RGS domain of two RGS domain containing proteins provide interfaces that allow for the formation of dimers of the said two RGS domain containing proteins. Individual RGS domains provide interfaces that allow for the formation of dimers between two separate RGS domain containing proteins. For example, the RGS domain between two RGS4 has been shown to homodimerize. In another example, the RGS domain in RGS9-2 can form homodimers. Non-limiting examples of RGS domain containing proteins are AXIN1, AXIN2 GRK1, GRK2, GRK3, GRK4, GRK5, GRK6, GRK7 RGS1, RGS2, RGS3, RGS4, RGS5, RGS6, RGS7, RGS8, RGS9, RGS10, RGS11, RGS12, RGS13, RGS14, RGS16, RGS17, RGS18, RGS19, RGS20, RGS21 and SNX13. The RGS domains of the RGS domain containing proteins presented herein can be used to homodimerize two fusion polypeptides. The RGS domains presented herein can be used to heterodimerize two fusion polypeptides. The RGS domains of the RGS domain containing proteins presented herein can be used to form homodimers of two of the same fusion polypeptides. The RGS domains of the RGS domain containing proteins presented herein can be used to form heterodimers of two different fusion polypeptides. The RGS domains of the RGS domain containing proteins presented herein can be used to homotrimerize three fusion polypeptides. The RGS domains of the RGS domain containing proteins presented herein can be used to heterotrimerize three fusion polypeptides. The RGS domains of the RGS domain containing proteins presented herein can be used to form homotrimers of three of the same fusion polypeptides. The RGS domains of the RGS domain containing proteins presented herein can be used to form heterotrimers of three different fusion polypeptides.

Additional non-limiting examples of linkers that can be used and their properties are further described in detail, e.g., in Chen X, Zaro J L, Shen W C. Fusion protein linkers: property, design and functionality. Adv Drug Deliv Rev. 2013; 65 (10): 1357-1369. doi: 10.1016/j.addr.2012.09.039; O'Shea E K, Lumb K J, Kim P S. Peptide ‘Velcro’: design of a heterodimeric coiled coil. Curr Biol. 1993 Oct. 1; 3 (10): 658-67. doi: 10.1016/0960-9822 (93) 90063-t. PMID: 15335856; and Müller K M, Arndt K M, Alber T. Protein fusions to coiled-coil domains. Methods Enzymol. 2000; 328:261-82. doi: 10.1016/s0076-6879 (00) 28402-4. PMID: 11075350, the contents of which are incorporated herein by reference in their entireties.

In some embodiments of any of the aspects provided herein, the linker comprises a polypeptide or a fragment thereof selected from the group consisting of: Table 5. In some embodiments of any of the aspects provided herein, the linker comprises a nucleic acid sequence encoding said linker selected from the group consisting of: Table 5. Non-limiting examples of nucleic acid sequences and amino acid sequences of linkers are provided in Table 5.

TABLE 5
Linkers

| | Nucleic Acid Sequence (SEQ ID NO:) |
|---|---|
| Linker | Amino Acid Sequence (SEQ ID NO:) |
| Human IgG | >AB776838.1 Homo sapiens mRNA for IgG H chain, complete cds, |
| heavy chain | clone: 231H5A11H, mRNA, nucleic acid sequence |
| (Bold Fc | ATGGAGTTTGGGCTGAGCTGGCTTTTTCTTGTGGCGATTTTAAAAGGTGTCCAGT |
| domain) | GTGAGGTGCAGTTGTTGGAGTCTGGGGGAGACTTGGTACAGCCTGGGGGGTCC |
| | CTGAGACTCTCCTGTGCAGCCTCTGGATTCACCTTTAGCACTTATGCCATGAGCT |
| | GGGTCCGCCAGGCTCCAGGGAAGGGGCTGGAATGGGTCTCAGGTATTGGTGAT |
| | AGTGGTCATAGCATATACTATGCAGACTCCGTGAAGGGCCGCTTCACCATCTCC |
| | AGAGACAATTCCAAGAACACGCTGTATCTGCAAATGAATAGCCTGAGAGCCGA |
| | GGACACGGCCGTATATTACTGTGCGACCGGCTCTCAGTGGCCGGGAGACTACTG |
| | GGGCCAGGGAACCCTGGTCACCGTCTCCTCAGCCTCCACCAAGGGCCCATCGGT |
| | CTTCCCCCTGGCACCCTCCTCCAAGAGCACCTCTGGGGGCACAGCGGCCCTGGG |
| | CTGCCTGGTCAAGGACTACTTCCCCGAACCGGTGACGGTGTCGTGGAACTCAGG |
| | CGCCCTGACCAGCGGCGTGCACACCTTCCCGGCTGTCCTACAGTCCTCAGGACT |
| | CTACTCCCTCAGCAGCGTGGTGACCGTGCCCTCCAGCAGCTTGGGCACCCAGAC |
| | CTACATCTGCAACGTGAATCACAAGCCCAGCAACACCAAGGTGGACAAGAGAG |
| | TTGAGCCCAAATCTTGTGACAAAACTCACACATGCCCACCGTGCCCAGCAC |
| | CTGAACTCCTGGGGGGACCGTCAGTCTTCCTCTTCCCCCCAAAACCCAAGG |
| | ACACCCTCATGATCTCCCGGACCCCTGAGGTCACATGCGTGGTGGTGGAC |
| | GTGAGCCACGAAGACCCTGAGGTCAAGTTCAACTGGTACGTGGACGGCGT |
| | GGAGGTGCATAATGCCAAGACAAAGCCGCGGGAGGAGCAGTACAACAGCA |
| | CGTACCGTGTGGTCAGCGTCCTCACCGTCCTGCACCAGGACTGGCTGAAT |
| | GGCAAGGAGTACAAGTGCAAGGTCTCCAACAAAGCCCTCCCAGCCCCCAT |
| | CGAGAAAACCATCTCCAAAGCCAAAGGGCAGCCCCGAGAACCACAGGTGT |
| | ACACCCTGCCCCCATCCCGGGATGAGCTGACCAAGAACCAGGTCAGCCTG |
| | ACCTGCCTGGTCAAAGGCTTCTATCCCAGCGACATCGCCGTGGAGTGGGA |
| | GAGCAATGGGCAGCCGGAGAACAACTACAAGACCACGCCTCCCGTGCTGG |
| | ACTCCGACGGCTCCTTCTTCCTCTACAGCAAGCTCACCGTGGACAAGAGCA |
| | GGTGGCAGCAGGGGAACGTCTTCTCATGCTCCGTGATGCATGAGGCTCTG |
| | CACAACCACTACACGCAGAAGAGCCTCTCCCTGTCTCCGGGTAAATGA |
| | (SEQ ID NO: 141) |
| | >AB776838.1 Homo sapiens mRNA for IgG H chain, complete cds, |
| | clone: 231H5A11H, amino acid sequence |
| | MEFGLSWLFLVAILKGVQCEVQLLESGGDLVQPGGSLRLSCAASGFTFSTYAMSW |
| | VRQAPGKGLEWVSGIGDSGHSIYYADSVKGRFTISRDNSKNTLYLQMNSLRAEDTA |
| | VYYCATGSQWPGDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTYICNVNH |
| | KPSNTKVDKRVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDELTKNQV |
| | SLTCLVKGFYPSDIAVEWESNGQPENNYKTTPPVLDSDGSFFLYSKLTVDKSR |
| | WQQGNVFSCSVMHEALHNHYTQKSLSLSPGK (SEQ ID NO: 142) |
| Human IgG | >KY053479.1 Synthetic construct Fc-adiponectin gene, complete |
| heavy chain | cds, mRNA, nucleic acid sequence |
| | ATGTACAGGATGCAACTCCTGTCTTGCATTGCACTAAGTCTTGCACTTGTCACG |
| | AACTCGATATCGGCCATGGTTAGATCTGACAAAACTCACACATGCCCACCGTGC |
| | CCAGCACCTGAACTCCTGGGGGGACCGTCAGTCTTCCTCTTCCCCCCAAAACCC |
| | AAGGACACCCTCATGATCTCCCGGACCCCTGAGGTCACATGCGTGGTGGTGGAC |
| | GTGAGCCACGAAGACCCTGAGGTCAAGTTCAACTGGTACGTGGACGGCGTGGA |
| | GGTGCATAATGCCAAGACAAAGCCGCGGGAGGAGCAGTACAACAGCACGTACC |
| | GTGTGGTCAGCGTCCTCACCGTCCTGCACCAGGACTGGCTGAATGGCAAGGAGT |
| | ACAAGTGCAAGGTCTCCAACAAAGCCCTCCCAGCCCCCATCGAGAAAACCATC |
| | TCCAAAGCCAAAGGGCAGCCCCGAGAACCACAGGTGTACACCCTGCCCCCATC |
| | CCGGGAGGAGATGACCAAGAACCAGGTCAGCCTGACCTGCCTGGTCAAAGGCT |
| | TCTATCCCAGCGACATCGCCGTGGAGTGGGAGAGCAATGGGCAGCCGGAGAAC |
| | AACTACAAGACCACGCCTCCCGTGCTGGACTCCGACGGCTCCTTCTTCCTCTAC |
| | AGCAAGCTCACCGTGGACAAGAGCAGGTGGCAGCAGGGGAACGTCTTCTCATG |
| | CTCCGTGATGCACGAGGCTCTGCACAACCACTACACGCAGAAGAGCCTCTCCCT |
| | GTCTCCGGGTAAAGCCAGCGGAAGTGGCGGAGGAGGCGGTCCTGGAGAAGGTG |
| | CCTATGTATACCGCTCAGCATTCAGTGTGGGATTGGAGACTTACGTTACTATCC |
| | CCAACATGCCCATTCGCTTTACCAAGATCTTCTACAATCAGCAAAACCACTATG |
| | ATGGCTCCACTGGTAAATTCCACTGCAACATTCCTGGGCTGTACTACTTTGCCTA |
| | CCACATCACAGTCTATATGAAGGATGTGAAGGTCAGCCTCTTCAAGAAGGACA |
| | AGGCTATGCTCTTCACCTATGATCAGTACCAGGAAAATAATGTGGACCAGGCCT |
| | CCGGCTCTGTGCTCCTGCATCTGGAGGTGGGCGACCAAGTCTGGCTCCAGGTGT |
| | ATGGGGAAGGAGAGCGTAATGGACTCTATGCTGATAATGACAATGACTCCACC |
| | TTCACAGGCTTTCTTCTCTACCATGACACCAACTCTAGAAAGCTTCCTGGAGAA |
| | GGTGCCTATGTATACCGCTCAGCATTCAGTGTGGGATTGGAGACTTACGTTACT |
| | ATCCCCAACATGCCCATTCGCTTTACCAAGATCTTCTACAATCAGCAAAACCAC |
| | TATGATGGCTCCACTGGTAAATTCCACTGCAACATTCCTGGGCTGTACTACTTTG |
| | CCTACCACATCACAGTCTATATGAAGGATGTGAAGGTCAGCCTCTTCAAGAAGG |
| | ACAAGGCTATGCTCTTCACCTATGATCAGTACCAGGAAAATAATGTGGACCAGG |
| | CCTCCGGCTCTGTGCTCCTGCATCTGGAGGTGGGCGACCAAGTCTGGCTCCAGG |
| | TGTATGGGGAAGGAGAGCGTAATGGACTCTATGCTGATAATGACAATGACTCC |
| | ACCTTCACAGGCTTTCTTCTCTACCATGACACCAACACTAGTCCTGGAGAAGGT |
| | GCCTATGTATACCGCTCAGCATTCAGTGTGGGATTGGAGACTTACGTTACTATC |
| | CCCAACATGCCCATTCGCTTTACCAAGATCTTCTACAATCAGCAAAACCACTAT |
| | GATGGCTCCACTGGTAAATTCCACTGCAACATTCCTGGGCTGTACTACTTTGCCT |
| | ACCACATCACAGTCTATATGAAGGATGTGAAGGTCAGCCTCTTCAAGAAGGAC |
| | AAGGCTATGCTCTTCACCTATGATCAGTACCAGGAAAATAATGTGGACCAGGCC |
| | TCCGGCTCTGTGCTCCTGCATCTGGAGGTGGGCGACCAAGTCTGGCTCCAGGTG |
| | TATGGGGAAGGAGAGCGTAATGGACTCTATGCTGATAATGACAATGACTCCAC |
| | CTTCACAGGCTTTCTTCTCTACCATGACACCAACTAA (SEQ ID NO: 143) |
| | >KY053479.1 Synthetic construct Fc-adiponectin gene, |
| | complete cds, amino acid sequence |
| | PCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAP |
| | IEKTISKAKGQPREPQVYTLPPSREEMTKNQVSLTCLVKGFYPSDIA VEWES |
| | NGQPENNYKTTPPVLDSDGSFFLYSKLTVDKSRWQQGNVFSCSVMHEALHNH |
| | YTQKSLSLSPGK (SEQ ID NO: 144) |
| Fc-mutein | >Fc-mutein, mRNA, nucleic acid sequence |
| Domain: | CCGTGCCCAGCACCTGAAGCAGCCGGGGGACCGTCAGTCTTCCTCTTCCCCCCA |
| hIgG1 | AAACCCAAGGACACCCTCATGATCTCCCGGACCCCTGAGGTCACATGCGTGGTG |
| (P228-K447) | GTGGACGTGAGCCACGAAGACCCTGAGGTCAAGTTCAACTGGTACGTGGACGG |
| Bold: L234A | CGTGGAGGTGCATAATGCCAAGACAAAGCCGCGGGAGGAGCAGTACAACAGC |
| L235A P329G | ACGTACCGTGTGGTCAGCGTCCTCACCGTCCTGCACCAGGACTGGCTGAATGGC |
| | AAGGAGTACAAGTGCAAGGTCTCCAACAAAGCCCTCGGCGCCCCCATCGAGAA |
| | AACCATCTCCAAAGCCAAAGGGCAGCCCCGAGAACCACAGGTGTACACCCTGC |
| | CCCCATCCCGGGATGAGCTGACCAAGAACCAGGTCAGCCTGACCTGCCTGGTCA |
| | AAGGCTTCTATCCCAGCGACATCGCCGTGGAGTGGGAGAGCAATGGGCAGCCG |
| | GAGAACAACTACAAGACCACGCCTCCCGTGCTGGACTCCGACGGCTCCTTCTTC |
| | CTCTACAGCAAGCTCACCGTGGACAAGAGCAGGTGGCAGCAGGGGAACGTCTT |
| | CTCATGCTCCGTGATGCATGAGGCTCTGCACAACCACTACACGCAGAAGAGCCT |
| | CTCCCTGTCTCCGGGTAAA (SEQ ID NO: 145) |
| | >Fc-mutein, amino acid sequence |
| | PCPAPEAAGGPSVFLFPPKPKDTLMISRTPEVTCVVVDVSHEDPEVKFNWYVDGVE |
| | VHNAKTKPREEQYNSTYRVVSVLTVLHQDWLNGKEYKCKVSNKALGAPIEKTISK |
| | AKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK |
| | (SEQ ID NO: 146) |
| IgG2 | >AJ250170.1, Homo sapiens partial IgG2 gene for |
| | immunoglobulin heavy chain constant region IgG2, |
| | exons 1-4, DNA, nucleic acid sequence |
| | TGTACATAGTAGTTGTCTAGCACTGTCTCATAGTAGTAGTCATAGGTCGTCAGC |
| | GTCGTCTCAGTCACTGTCTCATCAGTCGTCAGCGTCTCATCATAGGTCACTTCAT |
| | AGTAGTAGAGCGTCGTCTCATAGTAGTAGGTCGTCGTCGTCAGCTCAACTGTCG |
| | TCGTCACTGTCTAGGTCGTCAGCGTCTCATAGACTACTGTCGTCTAGGTCACTAG |
| | CAGCAGCAGCAGCAGCAGCAGCATTTCTAGCGTCGTCTCAACTGTCGTCTAGGT |
| | CGTCTCAAGCGTCTCATCAACTGTCTCATCAACTGTCGTCAGCTAGGTCTCAACT |
| | TCAACTTCATCATCAACTAGCACTTAGGTCTCATCATCAGTCTAGGTCACTAGC |
| | GTCTCATCATCAACTGTCACTTCAACTTCAAGCTAGGTCGTCACTTCATCATCAT |
| | AGGTCTCAAGCAGCAGCAGCAGCAGCAGCTCTGCGTCTAGCTCATAGGTCGTCA |
| | CTTCATCATCATAGTCAAGCGTCTAGGTCGTCACTTAGACTGTCACTTCAAGCA |
| | CTACTGTCACTACTTCATCAGTCACTGTCAGCGTCGTCGTCTCATCATAGTCATA |
| | GGTCTCAAGCGTCTCATCATCATAGGTCGTCGTCTCATCAAGCTCAACTGTCTC |
| | ATAGTCATAGGTCTAGTCAAGCAGCAGCAGCAGCAGCAGCTCTAGGTCTAGCTC |
| | ATCAACTTCAACTTCATCAGTCTCAGTCAGCGTCTAGTCAACTTCAACTTAGGTC |
| | GTCTCAAGCACTTCATCAACTTCATCATAGTCATAGTCAAGCTAGTAGGTCTCA |
| | ACTGTCTCATCATAGTCAAGCTCAACTTCATCAACTACTGTCGTCGTCTCAAGCT |
| | CATCAACTTAGTCAGTCGTCTAGTCATAGAGCAGCAGCAGCAGCAGCAGCGCG |
| | AGATCTAGCTAGTCATCATCATCATCATAGGTCGTCTCAAGCGTCTCATCATCAT |
| | AGGTCTCATAGTCATCAAGCACTGTCGTCACTGTCTCAACTTCATCATAGAGCT |
| | CATCAGTCACTGTCACTGTCTCAACTTCAAGCACTGTCTCAGTCGTCTCATCATC |
| | ATAGGTCAGCGTCGTCTCATAGGTCTCATCATAGGTCGTCAGCAGCAGCAGCAG |
| | CAGCAGCGTGATATCTAGCTAGTCAACTACTGTCGTCACTTCATAGACTAGCTC |
| | ATAGTAGTCATCATCATCAGTCACTACTAGCTCATCAGTCGTCTAGGTCACTTCA |
| | GTCGTCAGCTAGGTCTAGTCAGTCTAGGTCGTCACTACTAGCTCATAGTCAACT |
| | GTCGTCTCAGTCTCATAGAGCTCATAGGTCACTTCATCAACTGTCTCAGTCAGC |
| | AGCAGCAGCAGCAGCAGCGTGATTTCTAGCGTCTCAGTCTAGGTCTCAACTTCA |
| | ACTTCAAGCTCATAGTAGTCATCATCAGTCGTCTCATAGAGCGTCTAGTCATCA |
| | TAGACTTCAACTGTCTAGAGCTCATCATAGTCAACTGTCGTCACTTCATAGAGC |
| | TCATAGACTTCATAGTCATCATCATAGTCAAGCACTGTCTCAACTGTCTCAGTCT |
| | AGGTCGTCAGCAGCAGCAGCAGCAGCAGCAGAGCGTCTAGCTAGGTCACTTCA |
| | TCAGTCTAGGTCACTTCAAGCTCATAGTCATCAACTGTCTCAACTACTTCAAGCT |
| | AGTAGTCAGTCGTCTCAACTTCATCATCAAGCACTGTCACTTCATCATAGACTTC |
| | AACTTCAAGCTCATAGGTCTCAACTACTTCAGTCTAGACTAGCGTCACTTAGTC |
| | AACTTCAACTACTGTCTCAAGCAGCAGCAGCAGCAGCAGCAGAAGGTCTAGCT |
| | CATCAACTGTCTCAACTACTTCAACTTCAAGCTCAACTACTGTCGTCTAGGTCGT |
| | CACTTCAAGCACTACTGTCACTTCAACTGTCTAGTAGGTCAGCGTCTAGGTCAC |
| | TGTCACTGTCGTCTCATCAAGCACTGTCTCATAGTCAACTGTCGTCGTCACTAGC |
| | GTCGTCGTCACTGTCGTCGTCTAGGTCTAGAGCAGCAGCAGCAGCAGCAGCCGC |
| | AGATCTAGCTCATAGGTCTCATAGGTCGTCACTACTGTCAGCTCATCAACTGTC |
| | GTCTCATAGTCAACTGTCAGCTCATCATCATAGTCATCATAGGTCTCATCAAGCT |
| | AGGTCGTCACTTCAGTCTCAACTTCATCAAGCTCATCAGTCGTCTCATAGGTCTA |
| | GGTCTCAAGCACTGTCTCATCATCATCAACTGTCTCATCAAGCAGCAGCAGCAG |
| | CAGCAGCATTATATCTAGCTCAACTGTCGTCGTCTCAACTGTCTCAACTAGCAC |
| | TGTCGTCTCAACTGTCGTCTCATCATCAAGCTCAACTTAGTCATAGGTCTAGTCA |
| | TAGTCAAGCTCATAGTCAACTTCATCATCAGTCGTCACTAGCGTCGTCTCATCAT |
| | AGTCATAGGTCTCATCAAGCTCAGTCTCATCATCATCAACTTCATAGTCAAGCA |
| | GCAGCAGCAGCAGCAGCATTATTTCTAGCACTTAGGTCTCATAGTCAACTGTCG |
| | TCGTCAGCACTGTCACTGTCGTCGTCTAGTCATAGTAGAGCTCATAGGTCGTCTC |
| | ATAGTAGTAGTAGTAGAGCTCATCAACTTCATCAACTGTCGTCTCATAGAGCTC |
| | ATCAACTGTCGTCTCAACTGTCGTCTCAAGCACTTCAACTGTCGTCTCATAGGTC |
| | GTCGTCAGCAGCAGCAGCAGCAGCAGCACCGCGTCTAGCTAGGTCTCATCATCA |
| | TCATAGACTTCATCAAGCTCATCAACTGTCGTCTCATCATCATAGTAGAGCTCA |
| | ACTTCAACTTCAACTTCAACTGTCGTCAGCGTCGTCTCAACTGTCGTCTAGGTCT |
| | CATAGAGCTAGGTCGTCTCATAGTCAACTGTCACTTCAAGCTCATAGGTCTCAT |
| | CAACTACTACTACTGTCAGCAGCAGCAGCAGCAGCAGCACCAGGTCTAGCTCAT |
| | CAACTTAGACTTAGTCATCAGTCGTCAGCGTCACTGTCGTCACTTCATCATCATA |
| | GGTCAGCTCATCATCATCATAGGTCACTTCATCATAGAGCACTACTGTCTCATC |
| | AGTCACTTCATCATCAAGCTCAACTACTACTGTCGTCTCATCAACTACTAGCACT |
| | TCATAGGTCTAGTCATCAACTTCATAGAGCAGCAGCAGCAGCAGCAGCAGGAG |
| | ATCTAGCTCATCATCATAGTCAACTGTCTCATAGTCAAGCGTCGTCACTTCAACT |
| | TCATCATAGTAGTCAAGCTAGTCATAGTCATCATAGTCATCATCAACTAGCGTC |
| | ACTTAGTCATCAGTCACTGTCTAGACTAGCACTTCATAGTCATCATCAACTACTT |
| | AGTCAAGCTAGTAGTCATAGTCATAGTCATAGGTCTCAAGCAGCAGCAGCAGC |
| | AGCAGCCAAATATCTAGCACTGTCACTGTCTCAGTCTCAACTACTACTAGCTAG |
| | GTCTAGTAGGTCTAGGTCTAGTCAGTCAGCACTGTCTAGGTCTCATCATCAACTT |
| | CATCAAGCGTCTAGGTCTCATCATCAACTGTCGTCTAGAGCACTACTGTCTCAT |
| | CAACTGTCTCATCATCAAGCACTGTCGTCTCATCATAGTCAGTCTCATCAAGCA |
| | GCAGCAGCAGCAGCAGCCAAATTTCTAGCTCATAGTCATCAACTGTCTCATAGT |
| | CAACTAGCACTGTCGTCTCAGTCGTCGTCACTTCAACTAGCGTCGTCTAGGTCTC |
| | ATCATCATAGACTGTCAGCACTGTCTAGACTGTCTCATCATAGGTCTCAAGCAC |
| | TTAGTCATCAACTGTCGTCGTCACTTCAAGCACTGTCGTCTCATCATCATCAACT |
| | GTCTCAAGCAGCAGCAGCAGCAGCTCTATAGCGTCTAGCTAGGTCGTCGTCTAG |
| | GTCTCATAGGTCACTAGCTCAACTTCAGTCTAGTCATCAACTTCATCAAGCTAGT |
| | CATCAACTTAGTCATAGTCATAGTAGAGCTCATCATAGTCAACTGTCTCAACTT |
| | CATCAAGCACTTCATCATAGGTCTAGGTCGTCTCAACTAGCGTCGTCACTTCAT |
| | CAGTCTAGTCAACTGTCAGCAGCAGCAGCAGCAGCTCTATAAGGTCTAGCTAGT |
| | CATAGTAGTCATCATAGTCATAGTAGAGCTCATCATCATCATCATCAACTACTA |
| | CTACTAGCTCATCATCAACTACTGTCGTCACTTCAACTAGCTCATCATCATAGTC |
| | AACTTAGGTCACTTAGAGCTCATAGTCATCATCAGTCGTCACTTCATCAAGCTC |
| | ATCATAGGTCACTGTCGTCTAGTCAACTAGCAGCAGCAGCAGCAGCTCTTCTAG |
| | ATCTAGCTCAGTCTAGGTCTCAGTCTAGGTCGTCTAGAGCGTCGTCTAGGTCGT |
| | CACTTCAGTCTAGGTCAGCACTGTCTCATCAACTTCAGTCACTACTGTCAGCACT |
| | TCATCATCATCAGTCACTGTCGTCTAGAGCTCATCAACTGTCTAGTAGTCAACTA |
| | CTTCAAGCTAGGTCGTCTAGACTTCAGTCTAGGTCGTCAGCAGCAGCAGCAGCA |
| | GCTCTGCGATATCTAGCACTTCAGTCGTCTCAACTTAGGTCGTCACTAGCGTCGT |
| | CTAGGTCTCAACTTAGACTACTTAGAGCGTCTCATCAACTACTGTCACTTCAACT |
| | ACTAGCACTGTCTCATCAACTTCAGTCGTCGTCACTAGCGTCGTCACTGTCTCAA |
| | CTGTCTAGTAGTCAAGCACTACTTCAACTGTCTCAACTTCAGTCTAGAGCAGCA |
| | GCAGCAGCAGCTCTGCGATTTCTAGCTAGTCATCAGTCTAGGTCTAGGTCGTCT |
| | AGAGCTCAACTGTCTCAGTCTAGTCATCATAGTCAAGCACTTCATCAGTCTAGT |
| | CAGTCTAGGTCTCAAGCACTTCATCAACTGTCGTCACTTCATAGGTCAGCGTCT |
| | CATAGGTCACTACTTCAGTCGTCTCAAGCACTACTGTCGTCACTGTCTAGACTTC |
| | AACTAGCAGCAGCAGCAGCAGCTCTGTGGCGTCTAGCACTGTCTAGGTCTCAAC |
| | TACTGTCGTCTAGAGCTCATAGTCATCAACTACTTCAACTACTACTAGCGTCGTC |
| | TCATCATAGTCATCATCAACTGTCAGCTCATCATCATCATCAACTTAGTCAGTCA |
| | CTAGCGTCACTACTACTACTTCATCAACTTAGTCAAGCTAGTCATCAACTACTA |
| | CTACTTCATCAACTAGCAGCAGCAGCAGCAGCTCTGTGAGGTCTAGCACTACTG |
| | TCGTCTAGGTCGTCGTCACTTCAAGCTCATCAGTCTCAGTCGTCGTCGTCTAGAC |
| | TAGCTAGGTCACTGTCGTCGTCTCATCAACTTCAAGCACTTAGGTCGTCACTTCA |
| | ACTGTCACTGTCAGCGTCTCATCAGTCGTCTCATAGTCAGTCGTCAGCTCATCAT |
| | CAACTTCATCATCATAGTCATAGAGCAGCAGCAGCAGCAGCTCTAGAAGATCTA |
| | GCGTCTCATCATCATAGGTCGTCGTCACTGTCAGCTAGGTCACTTCATCAGTCTC |
| | ATAGGTCTAGAGCGTCTCATCAACTACTTCATCATAGTCATAGAGCGTCTAGTC |
| | ATCATCATAGACTTCAACTGTCAGCGTCGTCTCAACTGTCTCATCATCATCAGTC |
| | AGCACTGTCACTACTTCATCAACTTCAACTGTCAGCAGCAGCAGCAGCAGCTCT |
| | CGCATATCTAGCGTCTAGGTCTAGACTTCAACTTCATCATCAAGCTAGGTCTCAT |
| | CATCATCATCAACTTAGTCAAGCTCATCAGTCGTCGTCACTGTCGTCACTGTCA |
| | GCACTTAGGTCACTTCATCAACTACTGTCACTAGCACTTCATCAACTGTCGTCTA |
| | GTCAACTGTCAGCTCATCATAGGTCACTTCATCATAGGTCTCAAGCAGCAGCAG |
| | CAGCAGCTCTCGCATTTCTAGCTCATAGGTCGTCTAGTCAACTACTACTGTCAG |
| | CGTCTCATAGTAGTCATAGACTTCATCATCAAGCTCAACTGTCTCAGTCACTTCA |
| | ACTTAGTCAAGCGTCTCATCAGTCTAGGTCGTCACTGTCTAGAGCGTCGTCGTC |
| | ACTGTCACTGTCTCAACTACTAGCTAGGTCGTCGTCTCAACTGTCTCATCAGTCA |
| | GCAGCAGCAGCAGCAGCTCTATTGCGTCTAGCGTCACTGTCACTACTTCAACTA |
| | CTTCATAGAGCACTTCAACTACTGTCACTTCATCAACTTCAAGCACTTCATCATA |
| | GTCATCATCAACTTAGGTCAGCTCATAGGTCGTCACTTCATAGTCATCAGTCAG |
| | CACTTCAGTCGTCTCATAGTCATCATAGTAGAGCTCATAGTAGTCATCATAGTC |
| | ATAGACTTCAAGCAGCAGCAGCAGCAGCTCTATTAGGTCTAGCACTGTCTCAAC |
| | TACTGTCTCATAGTCAACTAGCTCATCAGTCTAGGTCGTCACTTCAACTACTAGC |
| | GTCACTGTCTCAACTGTCGTCTAGGTCGTCAGCTCAACTGTCTCAACTGTCGTCG |
| | TCGTCACTAGCACTTCAGTCTAGTCATAGTAGTCATAGTCAAGCACTTAGGTCT |
| | CATAGTCATCAGTCTAGGTCAGCAGCAGCAGCAGCAGCTCTACCAGATCTAGCA |
| | CTTAGGTCTCAACTTAGGTCACTGTCGTCAGCTCATAGTCATAGGTCTCAACTTC |
| | AACTACTAGCTCATCAACTTCATAGACTTCAACTTCAACTAGCTCAACTGTCAC |
| | TACTGTCACTGTCTCATCAAGCTAGTCATAGTCATCATCATAGGTCTAGTCAAG |
| | CTAGTCATCAGTCGTCGTCTAGACTACTACTAGCAGCAGCAGCAGCAGCTCTAG |
| | GATATCTAGCTAGGTCACTGTCTAGGTCTCATCAACTTCAAGCGTCGTCTCATCA |
| | ACTGTCTCAACTACTGTCAGCTCATCATCATCATCAGTCTCATAGTCATCAAGCT |
| | CATCAACTGTCGTCTCATAGTCATAGTCAAGCGTCGTCGTCGTCTAGTCAGTCTC |
| | AGTCTCAAGCGTCACTGTCGTCACTTAGGTCTCATAGTAGAGCAGCAGCAGCAG |
| | CAGCTCTAGGATTTCTAGCGTCGTCTCAACTTCAGTCTAGACTTCATCAAGCTCA |
| | TCAGTCTAGGTCTAGACTTCAACTTAGAGCACTTCATAGTAGTCATCATCAGTC |
| | GTCGTCAGCTCAACTTCATCATCAACTGTCTCAACTTAGAGCGTCGTCACTACT |
| | ACTTAGACTACTACTGTCAGCTCAACTTCATCATCAACTGTCTCAGTCTCAAGC |
| | AGCAGCAGCAGCAGCTCTCAAGCGTCTAGCTAGTAGTCATCATCATAGGTCGTC |
| | GTCTCAAGCTCATCATCATAGGTCTCAGTCACTGTCACTAGCTCATAGGTCTAG |
| | GTCACTTAGGTCGTCTAGAGCTAGTCATAGTAGTAGTCATCAACTTCAGTCAGC |
| | GTCGTCTAGTCAACTGTCGTCTCATCAGTCAGCACTGTCTAGTCATAGGTCACTG |
| | TCGTCTCAAGCAGCAGCAGCAGCAGCTCTCAAAGGTCTAGCTCATAGGTCACTG |
| | TCTAGGTCGTCTCAACTAGCTAGGTCACTGTCGTCGTCACTGTCGTCTCAAGCA |
| | CTGTCACTGTCTAGGTCGTCGTCTAGTCATAATCAGTAGAG (SEQ ID NO: 147) |
| | >AJ250170.1, Homo sapiens partial IgG2 gene for immunoglobulin |
| | heavy chain constant region IgG2, exons 1-4, amino acid sequence |
| | ALTSGVHTFPAVLQSSGLYSLSSVVTVTSSNFGTQTYTCNVDHKPSNTKVDKTVER |
| | KCCVECPPCPAPPVAGPSVFLFPPKPKDTLMISRTPEVTCVVVDVSHEDPEVQFNW |
| | YVDGMEVHNAKTKPREEQFNSTFRVVSVLTVVHQDWLNGKEYKCKVSNKGLPAP |
| | IEKTISKTKGQPREPQVYTLPPSREEMTKNQVSLTCLVKGFYPSDIAVEWESNGQPE |
| | NNYKTTPPMLDSDGSFFLYSKLTVDKSRWQQGNVFSCSVMHEALHNHYTQKSLSL |
| | SPGK (SEQ ID NO: 148) |
| IgG3 | >FJ200489.1, Homo sapiens immunoglobulin heavy chain |
| | gamma-3 variant mRNA, complete cds, nucleic acid sequence |
| | ATGGAGTTTGGGCTGAGCTGGGTTCTCCTTGTTGTTTTTTTACAAGGTGTCCAGT |
| | GTGAGGTGCAACTGGTGGACTCTGGGGGAGGCTTGGTCCAGCCTGGAGGGTCC |
| | CTGAGACTCTCGTGTGCAGCCTCTGGATTCATCGTCAGTGACCACTACGTAGAG |
| | TGGGTCCGCCAGGCTCCAGGGAAGGGGCCGGAGTGGGTTGGTTGTTTCAGAAG |
| | CAAAGCTCACAAGTCCACCACAGAATATGCCGCGTCTGTGAAAGGCAGATTCA |
| | CCATCTTAAGAGATGATTCGAAGAACTCAGTGCATCTCCAAATGAACAGCCTCA |
| | AAACCGACGACACGGCCGTGTATTATTGTGTTAGAGATCTTGAGGGGGCTGGTA |
| | AATACGACTGGTATTTCGATATTTGGGGCCGAGGCATCCTGGTCACTGTCTCCT |
| | CAGCTTCCACCAAGGGCCCATCGGTCTTCCCCCTGGCGCCCTGCTCCAGGAGCA |
| | CCTCTGGGGGCACAGCGGCCCTGGGCTGCCTGGTCAAGGACTACTTCCCCGAAC |
| | CGGTGACGGTGTCGTGGAACTCAGGCGCCCTGACCAGCGGCGTGCACACCTTCC |
| | CGGCTGTCCTACAGTCCTCAGGACTCTACTCCCTCAGCAGCGTGGTGACCGTGC |
| | CCTCCAGCAGCTTGGGCACCCAGACCTACACCTGCAACGTGAATCACAAGCCCA |
| | GCAACACCAAGGTGGACAAGAGAGTTGAGCCCAAATCTTGTGACACACCTCCC |
| | CCATGCCCACGGTGCCCAGCACCTGAACTCCTGGGAGGACCGTCAGTCTTCCTC |
| | TTCCCCCCAAAACCCAAGGATACCCTTATGATTTCCCGGACCCCTGAGGTCACG |
| | TGCGTGGTGGTGGACGTGAGCCACGAAGACCCCGAGGTCCAGTTCAAGTGGTA |
| | CGTGGACGGCGTGGAGGTGCATAATGCCAAGACAAAGCCGCGGGAGGAGCAGT |
| | TCAACAGCACGTTCCGTGTGGTCAGCGTCCTCACCGTCCTGCACCAGGACTGGC |
| | TGAACGGCAAGGAGTACAAGTGCAAGGTCTCCAACAAAGCCCTCCCAGCCCCC |
| | ATCGAGAAAACCATCTCCAAAACCAAAGGACAGCCCCGAGAACCACAGGTGTA |
| | CACCCTGCCCCCATCCCGGGAGGAGATGACCAAGAACCAGGTCAGCCTGACCT |
| | GCCTGGTCAAAGGCTTCTACCCCAGCGACATCGCCGTGGAGTGGGAGAGCAGC |
| | GGGCAGCCGGAGAACAACTACAACACCACGCCTCCCATGCTGGACTCCGACGG |
| | CTCCTTCTTCCTCTACAGCAAGCTCACCGTGGACAAGAGCAGGTGGCAGCAGGG |
| | GAACATCTTCTCATGCTCCGTGATGCATGAGGCTCTGCACAACCGCTTCACGCA |
| | GAAGAGCCTCTCCCTGTCTCCGGGTAAATGA (SEQ ID NO: 149) |
| | >FJ200489.1, Homo sapiens immunoglobulin heavy chain |
| | gamma-3 variant mRNA, complete cds, amino acid sequence |
| | MEFGLSWVLLVVFLQGVQCEVQLVDSGGGLVQPGGSLRLSCAASGFIVSDHYVEW |
| | VRQAPGKGPEWVGCFRSKAHKSTTEYAASVKGRFTILRDDSKNSVHLQMNSLKTD |
| | DTAVYYCVRDLEGAGKYDWYFDIWGRGILVTVSSASTKGPSVFPLAPCSRSTSGGT |
| | AALGCLVKDYFPEPVTVSWNSGALTSGVHTFPAVLQSSGLYSLSSVVTVPSSSLGT |
| | QTYTCNVNHKPSNTKVDKRVEPKSCDTPPPCPRCPAPELLGGPSVFLFPPKPKDTLM |
| | ISRTPEVTCVVVDVSHEDPEVQFKWYVDGVEVHNAKTKPREEQFNSTFRVVSVLT |
| | VLHQDWLNGKEYKCKVSNKALPAPIEKTISKTKGQPREPQVYTLPPSREEMTKNQV |
| | SLTCLVKGFYPSDIAVEWESSGQPENNYNTTPPMLDSDGSFFLYSKLTVDKSRWQQ |
| | GNIFSCSVMHEALHNRFTQKSLSLSPGK (SEQ ID NO: 150) |
| IgG4 (4Fc) | >Synthetic construct IgG4 Fc domain, DNA, nucleic acid sequence |
| | GAGTCCAAATATGGTCCCCCATGCCCATCATGCCCAGCACCTGAGTTCCTGGGG |
| | GGACCATCAGTCTTCCTGTTCCCCCCAAAACCCAAGGACACTCTCATGATCTCC |
| | CGGACCCCTGAGGTCACGTGCGTGGTGGTGGACGTGAGCCAGGAAGACCCCGA |
| | GGTCCAGTTCAACTGGTACGTGGATGGCGTGGAGGTGCATAATGCCAAGACAA |
| | AGCCGCGGGAGGAGCAGTTCAACAGCACGTACCGTGTGGTCAGGGTCCTCACC |
| | GTCCTGCACCAGGACTGGCTGAACGGTAAGGAGTACAAGTGCAAGGTCTCCAA |
| | CAAAGGCCTCCCGTCCTCCATCGAGAAAACCATCTCCAAAGCCAAAGGGCAGC |
| | CCCGAGAGCCACAGGTGTACACCCTGCCCCCATCCCAGGAGGAGATGACCAAG |
| | AACCAGGTCAGCCTGACCTGCCTGGTCAAAGGCTTCTACCCCAGCGACATCGCC |
| | GTGGAGTGGGAGAGCAATGGGCAGCCGGAGGACAACTACAAGACCACGCCTCC |
| | CGTGCTGGACTCCGACGGCTCCTTCTTCCTCTACAGCAGGCTAACCGTGGACAA |
| | GAGCAGGTGGCAGGAGGGGAATGTCTTCTCATGCTCCGTGATGCATGAGGCTCT |
| | GCACAACCACTACACACAGAAGAGCCTCTCCCTGTCTCCGGGTAAA (SEQ ID |
| | NO: 151) |
| | >Synthetic construct IgG4 Fc domain, amino acid sequence |
| | MRIFAVFIFMTYWHLLNAFTVTVPKDLYVVEYGSNMTIECKFPVEKQLDLAALIVY |
| | WEMEDKNIIQFVHGEEDLKVQHSSYRQRARLLKDQLSLGNAALQITDVKLQDAGV |
| | YRCMISYGGADYKRITVKVNAPYNKINQRILVVDPVTSEHELTCQAEGYPKAEVIW |
| | TSSDHQVLSGKTTTTNSKREEKLFNVTSTLRINTTTNEIFYCTFRRLDPEENHTAELV |
| | IPELPLAHPPNERESKYGPPCPSCPAPEFLGGPSVFLFPPKPKDTLMISRTPEVTCVVV |
| | DVSQEDPEVQFNWYVDGVEVHNAKTKPREEQFNSTYRVVRVLTVLHQDWLNGKE |
| | YKCKVSNKGLPSSIEKTISKAKGQPREPQVYTLPPSQEEMTKNQVSLTCLVKGFYPS |
| | DIAVEWESNGQPEDNYKTTPPVLDSDGSFFLYSRLTVDKSRWQEGNVFSCSVMHE |
| | ALHNHYTQKSLSLSPGKFYTGVYILIGAGALMMLVGFLGCCGAVQESQCVIM |
| | (SEQ ID NO: 152) |
| G Linker A | >G Linker, nucleic acid sequence |
| | GGC |
| | >G Linker, amino acid sequence |
| | G |
| G Linker B | >G Linker, nucleic acid sequence |
| | GGT |
| | >G Linker, amino acid sequence |
| | G |
| G Linker C | >G Linker, nucleic acid sequence |
| | GGA |
| | >G Linker, amino acid sequence |
| | G |
| G Linker D | >G Linker, nucleic acid sequence |
| | GGG |
| | >G Linker, amino acid sequence |
| | G |
| GSSG Linker | >GSSG Linker, nucleic acid sequence |
| | GGCTCGAGCGGC (SEQ ID NO: 153) |
| | >GSSG Linker, amino acid sequence |
| | GSSG (SEQ ID NO: 154) |
| (GGGGS)1 | >(GGGGS)1 Linker, nucleic amino acid sequence |
| Linker A | GGTGGCGGTGGATCC (SEQ ID NO: 155) |
| | >(GGGGS)1 Linker, amino acid sequence |
| | GGGGS (SEQ ID NO: 156) |
| (GGGGS)1 | >(GGGGS)1 Linker, nucleic acid sequence |
| Linker B | GGTGGCGGTGGCTCC (SEQ ID NO: 157) |
| | >(GGGGS)1 Linker, amino acid sequence |
| | GGGGS (SEQ ID NO: 156) |
| (GGGGS)1 | >(GGGGS)1 Linker, nucleotide sequence |
| Linker C | GGAGGTGGCGGCTCC (SEQ ID NO: 158) |
| | >(GGGGS)1 Linker, amino acid sequence |
| | GGGGS (SEQ ID NO: 156) |
| (GGGGS)2 | >(GGGGS)2 Linker, nucleic acid sequence |
| Linker A | GGTGGCGGTGGCTCCGGTGGCGGTGGATCC (SEQ ID NO: 159) |
| | >(GGGGS)2 Linker, peptide sequence |
| | GGGGSGGGGS (SEQ ID NO: 160) |
| (GGGGS)2 | >(GGGGS)2 Linker, nucleic acid sequence |
| Linker B | GGTGGCGGTGGCTCCGGTGGCGGTGGCTCC (SEQ ID NO: 161) |
| | >(GGGGS)2 Linker, amino acid sequence |
| | GGGGSGGGGS (SEQ ID NO: 160) |
| (GGGGS)2 | >(GGGGS)2 Linker, nucleic acid sequence |
| Linker C | GGTGGCGGTGGCTCCGGAGGTGGCGGCTCC (SEQ ID NO: 162) |
| | >(GGGGS)2 Linker, amino acid sequence |
| | GGGGSGGGGS (SEQ ID NO: 160) |
| (GGGGS)2 | >(GGGGS)2 Linker, nucleic acid sequence |
| Linker D | GGTGGCGGTGGCTCCGGAGGTGGCGGTTCC |
| | (SEQ ID NO: 163) |
| | >(GGGGS)2 Linker, amino acid sequence |
| | GGGGSGGGGS (SEQ ID NO: 160) |
| (GGGGS)3 | >(GGGGS)3 Linker, nucleic acid sequence |
| Linker A | GGTGGCGGTGGCTCCGGTGGCGGTGGCTCCGGTGGCGGTGGATCC |
| | (SEQ ID NO: 164) |
| | >(GGGGS)3 Linker, amino acid sequence |
| | GGGGSGGGGSGGGGS (SEQ ID NO: 165) |
| (GGGGS)3 | >(GGGGS)4 Linker, nucleic acid sequence |
| Linker B | GGAGGTGGCGGTTCCGGTGGCGGTGGCTCCGGTGGCGGTGGCTCC |
| | (SEQ ID NO: 166) |
| | >(GGGGS)4 Linker, amino acid sequence GGGGSGGGGGGGGS |
| | (SEQ ID NO: 165) |
| (GGGGS)3 | >(GGGGS)4 Linker, nucleic acid sequence |
| Linker C | GGTGGCGGTGGCTCCGGTGGCGGTGGCTCCGGAGGTGGCGGCTCC |
| | (SEQ ID NO: 167) |
| | >(GGGGS)4 Linker, amino acid sequence |
| | GGGGSGGGGSGGGGS |
| | (SEQ ID NO: 165) |
| (GGGGS)4 | >(GGGGS)4 Linker, nucleic acid sequence |
| Linker A | GGTGGCGGTGGCTCCGGTGGCGGTGGCTCCGGTGGCGGTGGCTCCGGTGGCGG |
| | TGGATCC (SEQ ID NO: 168) |
| | >(GGGGS)4 Linker, amino acid sequence |
| | GGGGSGGGGSGGGGSGGGGS (SEQ ID NO: 169) |
| (GGGGS)4 | >(GGGGS)4 Linker, nucleic acid sequence |
| Linker B | GGTGGCGGTGGCTCCGGAGGTGGCGGTTCCGGTGGCGGTGGCTCCGGTGGCGG |
| | TGGCTCC (SEQ ID NO: 170) |
| | >(GGGGS)4 Linker, amino acid sequence |
| | GGGGSGGGGSGGGGSGGGGS |
| | (SEQ ID NO: 169) |
| (GGGGS)4 | >(GGGGS)4 Linker, nucleic acid sequence |
| Linker C | GGTGGCGGTGGCTCCGGTGGCGGTGGCTCCGGTGGCGGTGGCTCCGGAGGTGG |
| | CGGCTCC (SEQ ID NO: 171) |
| | >(GGGGS)4 Linker, amino acid sequence |
| | GGGGGGGGSGGGGSGGGGS (SEQ ID NO: 169) |
| ID | >ID Linker (Clal restriction enzyme cute site), |
| | nucleic acid sequence |
| | ATCGAT |
| | >ID Linker Clal restriction enzyme cute site), |
| | amino acid sequence |
| | ID |
| P2A Linker | >P2A Linker (Artificial sequence), nucleic acid sequence |
| | GCTACTAACTTCAGCCTGCTGAAGCAG (SEQ ID NO: 172) |
| | >P2A Linker (Artificial sequence), amino acid sequence |
| | ATNFSLKQAGDVENPGP (SEQ ID NO: 173) |

### Fusion Polypeptide Embodiments—Full Length Constructs

The engineered extracellular vesicle compositions provided herein can comprise variations in the configuration of the POI domain, linkers, and/or vesicle targeting domain. The specific combination and localization of these domains can enhance fusion polypeptide anchoring, function, or therapeutic effect, e.g., modulating the immune system, inducing activation or suppression of an immune cell, agonizing a target protein or receptor, etc. The engineered extracellular vesicle compositions provided herein can comprise fusion polypeptide variations in any aspect as presented herein. Non-limiting examples of nucleic acid sequences of fusion polypeptides and amino acid sequences of fusion polypeptides are provided in Table 6. In some embodiments of any of the aspects provided herein, the fusion polypeptide is a nucleic acid sequence encoding said fusion polypeptide selected from Table 6. In some embodiments of any of the aspects provided herein, the fusion polypeptide is an amino acid sequence of a fusion polypeptide selected from Table 6.

TABLE 6
Fusion Polypeptide Embodiments - Full Length Constructs

| Fusion | Nucleic Acid Sequence (SEQ ID NO:) |
|---|---|
| Polypeptide | Amino Acid Sequence (SEQ ID NO:) |
| Artificial | >Artificial sequence, M/P-4F2-Fc-sc41BBL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| sc41BBL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCCGCGAGGGTCC |
| | CGAGCTTTCGCCCGACGATCCCGCCGGCCTCTTGGACCTGCGGCAGGGCATGTT |
| | TGCGCAGCTGGTGGCCCAAAATGTTCTGCTGATCGATGGGCCCCTGAGCTGGTA |
| | CAGTGACCCAGGCCTGGCAGGCGTGTCCCTGACGGGGGGCCTGAGCTACAAAG |
| | AGGACACGAAGGAGCTGGTGGTGGCCAAGGCTGGAGTCTACTATGTCTTCTTTC |
| | AACTAGAGCTGCGGCGCGTGGTGGCCGGCGAGGGCTCAGGCTCCGTTTCACTTG |
| | CGCTGCACCTGCAGCCACTGCGCTCTGCTGCTGGGGCCGCCGCCCTGGCTTTGA |
| | CCGTGGACCTGCCACCCGCCTCCTCCGAGGCTCGGAACTCGGCCTTCGGTTTCC |
| | AGGGCCGCTTGCTGCACCTGAGTGCCGGCCAGCGCCTGGGCGTCCATCTTCACA |
| | CTGAGGCCAGGGCACGCCATGCCTGGCAGCTTACCCAGGGCGCCACAGTCTTG |
| | GGACTCTTCCGGGTGACCCCCGAAATCCCAGCCGGACTCCCTTCACCGAGGTCG |
| | GAAGGTGGCGGTGGCTCCGGAGGTGGCGGTTCCGGTGGCGGTGGCTCCGGTGG |
| | CGGTGGCTCCCGCGAGGGTCCCGAGCTTTCGCCCGACGATCCCGCCGGCCTCTT |
| | GGACCTGCGGCAGGGCATGTTTGCGCAGCTGGTGGCCCAAAATGTTCTGCTGAT |
| | CGATGGGCCCCTGAGCTGGTACAGTGACCCAGGCCTGGCAGGCGTGTCCCTGAC |
| | GGGGGGCCTGAGCTACAAAGAGGACACGAAGGAGCTGGTGGTGGCCAAGGCT |
| | GGAGTCTACTATGTCTTCTTTCAACTAGAGCTGCGGCGCGTGGTGGCCGGCGAG |
| | GGCTCAGGCTCCGTTTCACTTGCGCTGCACCTGCAGCCACTGCGCTCTGCTGCT |
| | GGGGCCGCCGCCCTGGCTTTGACCGTGGACCTGCCACCCGCCTCCTCCGAGGCT |
| | CGGAACTCGGCCTTCGGTTTCCAGGGCCGCTTGCTGCACCTGAGTGCCGGCCAG |
| | CGCCTGGGCGTCCATCTTCACACTGAGGCCAGGGCACGCCATGCCTGGCAGCTT |
| | ACCCAGGGCGCCACAGTCTTGGGACTCTTCCGGGTGACCCCCGAAATCCCAGCC |
| | GGACTCCCTTCACCGAGGTCGGAAGGTGGCGGTGGCTCCGGTGGCGGTGGCTCC |
| | GGTGGCGGTGGCTCCGGAGGTGGCGGCTCCCGCGAGGGTCCCGAGCTTTCGCCC |
| | GACGATCCCGCCGGCCTCTTGGACCTGCGGCAGGGCATGTTTGCGCAGCTGGTG |
| | GCCCAAAATGTTCTGCTGATCGATGGGCCCCTGAGCTGGTACAGTGACCCAGGC |
| | CTGGCAGGCGTGTCCCTGACGGGGGGCCTGAGCTACAAAGAGGACACGAAGGA |
| | GCTGGTGGTGGCCAAGGCTGGAGTCTACTATGTCTTCTTTCAACTAGAGCTGCG |
| | GCGCGTGGTGGCCGGCGAGGGCTCAGGCTCCGTTTCACTTGCGCTGCACCTGCA |
| | GCCACTGCGCTCTGCTGCTGGGGCCGCCGCCCTGGCTTTGACCGTGGACCTGCC |
| | ACCCGCCTCCTCCGAGGCTCGGAACTCGGCCTTCGGTTTCCAGGGCCGCTTGCT |
| | GCACCTGAGTGCCGGCCAGCGCCTGGGCGTCCATCTTCACACTGAGGCCAGGGC |
| | ACGCCATGCCTGGCAGCTTACCCAGGGCGCCACAGTCTTGGGACTCTTCCGGGT |
| | GACCCCCGAAATCCCAGCCGGACTCCCTTCACCGAGGTCGGAATAA |
| | (SEQ ID NO: 174) |
| | >Artificial sequence, M/P-4F2-Fc-sc41BBL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSREGPELSPDDPAGLLDLRQGMFAQLVAQNVLLID |
| | GPLSWYSDPGLAGVSLTGGLSYKEDTKELVVAKAGVYYVFFQLELRRVVAGEGSG |
| | SVSLALHLQPLRSAAGAAALALTVDLPPASSEARNSAFGFQGRLLHLSAGQRLGVH |
| | LHTEARARHAWQLTQGATVLGLFRVTPEIPAGLPSPRSEGGGGSGGGGSGGGGSG |
| | GGGSREGPELSPDDPAGLLDLRQGMFAQLVAQNVLLIDGPLSWYSDPGLAGVSLT |
| | GGLSYKEDTKELVVAKAGVYYVFFQLELRRVVAGEGSGSVSLALHLQPLRSAAGA |
| | AALALTVDLPPASSEARNSAFGFQGRLLHLSAGQRLGVHLHTEARARHAWQLTQG |
| | ATVLGLFRVTPEIPAGLPSPRSEGGGGSGGGGSGGGGSGGGGSREGPELSPDDPAGL |
| | LDLRQGMFAQLVAQNVLLIDGPLSWYSDPGLAGVSLTGGLSYKEDTKELVVAKAG |
| | VYYVFFQLELRRVVAGEGSGSVSLALHLQPLRSAAGAAALALTVDLPPASSEARNS |
| | AFGFQGRLLHLSAGQRLGVHLHTEARARHAWQLTQGATVLGLFRVTPEIPAGLPSP |
| | RSE (SEQ ID NO: 175) |
| Artificial | >Artificial sequence, M/P-4F2-Fc-scGITRL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| ScGITRL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGAGCCCTGTATG |
| | GCTAAGTTTGGACCATTACCCTCAAAATGGCAAATGGCATCTTCTGAACCTCCT |
| | TGCGTGAATAAGGTGTCTGACTGGAAGCTGGAGATACTTCAGAATGGCTTATAT |
| | TTAATTTATGGCCAAGTGGCTCCCAATGCAAACTACAATGATGTAGCTCCTTTT |
| | GAGGTGCGGCTGTATAAAAACAAAGACATGATACAAACTCTAACAAACAAATC |
| | TAAAATCCAAAATGTAGGAGGGACTTATGAATTGCATGTTGGGGACACCATAG |
| | ACTTGATATTCAACTCTGAGCATCAGGTTCTAAAAAATAATACATACTGGGGTA |
| | TCATTTTACTAGCAAATCCCCAATTCATCTCCGGTGGCGGTGGCTCCGGAGGTG |
| | GCGGTTCCGAGCCCTGTATGGCTAAGTTTGGACCATTACCCTCAAAATGGCAAA |
| | TGGCATCTTCTGAACCTCCTTGCGTGAATAAGGTGTCTGACTGGAAGCTGGAGA |
| | TACTTCAGAATGGCTTATATTTAATTTATGGCCAAGTGGCTCCCAATGCAAACT |
| | ACAATGATGTAGCTCCTTTTGAGGTGCGGCTGTATAAAAACAAAGACATGATAC |
| | AAACTCTAACAAACAAATCTAAAATCCAAAATGTAGGAGGGACTTATGAATTG |
| | CATGTTGGGGACACCATAGACTTGATATTCAACTCTGAGCATCAGGTTCTAAAA |
| | AATAATACATACTGGGGTATCATTTTACTAGCAAATCCCCAATTCATCTCCGGT |
| | GGCGGTGGCTCCGGTGGCGGTGGCTCCGAGCCCTGTATGGCTAAGTTTGGACCA |
| | TTACCCTCAAAATGGCAAATGGCATCTTCTGAACCTCCTTGCGTGAATAAGGTG |
| | TCTGACTGGAAGCTGGAGATACTTCAGAATGGCTTATATTTAATTTATGGCCAA |
| | GTGGCTCCCAATGCAAACTACAATGATGTAGCTCCTTTTGAGGTGCGGCTGTAT |
| | AAAAACAAAGACATGATACAAACTCTAACAAACAAATCTAAAATCCAAAATGT |
| | AGGAGGGACTTATGAATTGCATGTTGGGGACACCATAGACTTGATATTCAACTC |
| | TGAGCATCAGGTTCTAAAAAATAATACATACTGGGGTATCATTTTACTAGCAAA |
| | TCCCCAATTCATCTCCTAG (SEQ ID NO: 176) |
| | >Artificial sequence, M/P-4F2-Fc-scGITRL, amino sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSEPCMAKFGPLPSKWQMASSEPPCVNKVSDWKLEI |
| | LQNGLYLIYGQVAPNANYNDVAPFEVRLYKNKDMIQTLTNKSKIQNVGGTYELHV |
| | GDTIDLIFNSEHQVLKNNTYWGIILLANPQFISGGGGSGGGGSEPCMAKFGPLPSKW |
| | QMASSEPPCVNKVSDWKLEILQNGLYLIYGQVAPNANYNDVAPFEVRLYKNKDMI |
| | QTLTNKSKIQNVGGTYELHVGDTIDLIFNSEHQVLKNNTYWGIILLANPQFISGGGG |
| | SGGGGSEPCMAKFGPLPSKWQMASSEPPCVNKVSDWKLEILQNGLYLIYGQVAPN |
| | ANYNDVAPFEVRLYKNKDMIQTLINKSKIQNVGGTYELHVGDTIDLIFNSEHQVLK |
| | NNTYWGIILLANPQFIS |
| | (SEQ ID NO: 177) |
| Artificial | >Artificial sequence, M/P-4F2-Fc-scOX40L, mRNA, |
| Sequence | nucleotide sequence |
| M/P-4F2-Fc- | ATGGGTTGCTGTTTCTCCAAGACCGGCTCGAGCGGCAGCCAGGACACCGAGGT |
| scOX40L | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCCATCGGTATCCT |
| | CGAATTCAAAGTATCAAAGTACAATTTACCGAATATAAGAAGGAGAAAGGTTT |
| | CATCCTCACTTCCCAAAAGGAGGATGAAATCATGAAGGTGCAGAACAACTCAG |
| | TCATCATCAACTGTGATGGGTTTTATCTCATCTCCCTGAAGGGCTACTTCTCCCA |
| | GGAAGTCAACATTAGCCTTCATTACCAGAAGGATGAGGAGCCCCTCTTCCAACT |
| | GAAGAAGGTCAGGTCTGTCAACTCCTTGATGGTGGCCTCTCTGACTTACAAAGA |
| | CAAAGTCTACTTGAATGTGACCACTGACAATACCTCCCTGGATGACTTCCATGT |
| | GAATGGCGGAGAACTGATTCTTATCCATCAAAATCCTGGTGAATTCTGTGTCCT |
| | TGGTGGCGGTGGCTCCGGAGGTGGCGGTTCCCATCGGTATCCTCGAATTCAAAG |
| | TATCAAAGTACAATTTACCGAATATAAGAAGGAGAAAGGTTTCATCCTCACTTC |
| | CCAAAAGGAGGATGAAATCATGAAGGTGCAGAACAACTCAGTCATCATCAACT |
| | GTGATGGGTTTTATCTCATCTCCCTGAAGGGCTACTTCTCCCAGGAAGTCAACA |
| | TTAGCCTTCATTACCAGAAGGATGAGGAGCCCCTCTTCCAACTGAAGAAGGTCA |
| | GGTCTGTCAACTCCTTGATGGTGGCCTCTCTGACTTACAAAGACAAAGTCTACT |
| | TGAATGTGACCACTGACAATACCTCCCTGGATGACTTCCATGTGAATGGCGGAG |
| | AACTGATTCTTATCCATCAAAATCCTGGTGAATTCTGTGTCCTTGGTGGCGGTG |
| | GCTCCGGTGGCGGTGGCTCCCATCGGTATCCTCGAATTCAAAGTATCAAAGTAC |
| | AATTTACCGAATATAAGAAGGAGAAAGGTTTCATCCTCACTTCCCAAAAGGAG |
| | GATGAAATCATGAAGGTGCAGAACAACTCAGTCATCATCAACTGTGATGGGTTT |
| | TATCTCATCTCCCTGAAGGGCTACTTCTCCCAGGAAGTCAACATTAGCCTTCATT |
| | ACCAGAAGGATGAGGAGCCCCTCTTCCAACTGAAGAAGGTCAGGTCTGTCAAC |
| | TCCTTGATGGTGGCCTCTCTGACTTACAAAGACAAAGTCTACTTGAATGTGACC |
| | ACTGACAATACCTCCCTGGATGACTTCCATGTGAATGGCGGAGAACTGATTCTT |
| | ATCCATCAAAATCCTGGTGAATTCTGTGTCCTTTGA (SEQ ID NO: 178) |
| | >Artificial sequence, M/P-4F2-Fc-scOX40L, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSHRYPRIQSIKVQFTEYKKEKGFILTSQKEDEIMKVQ |
| | NNSVIINCDGFYLISLKGYFSQEVNISLHYQKDEEPLFQLKKVRSVNSLMVASLTYK |
| | DKVYLNVTTDNTSLDDFHVNGGELILIHQNPGEFCVLGGGGSGGGGSHRYPRIQSI |
| | KVQFTEYKKEKGFILTSQKEDEIMKVQNNSVIINCDGFYLISLKGYFSQEVNISLHYQ |
| | KDEEPLFQLKKVRSVNSLMVASLTYKDKVYLNVTTDNTSLDDFHVNGGELILIHQN |
| | PGEFCVLGGGGSGGGGSHRYPRIQSIKVQFTEYKKEKGFILTSQKEDEIMKVQNNSV |
| | IINCDGFYLISLKGYFSQEVNISLHYQKDEEPLFQLKKVRSVNSLMVASLTYKDKVY |
| | LNVTTDNTSLDDFHVNGGELILIHQNPGEFCVL (SEQ ID NO: 179) |
| Artificial | >Artificial sequence, M/P-4F2-Fc-scCD27L, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scCD27L | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCCTTGGGTGGGA |
| | CGTAGCTGAGCTGCAGCTGAATCACACAGGACCTCAGCAGGACCCCAGGCTAT |
| | ACTGGCAGGGGGGCCCAGCACTGGGCCGCTCCTTCCTGCATGGACCAGAGCTG |
| | GACAAGGGGCAGCTACGTATCCATCGTGATGGCATCTACATGGTACACATCCAG |
| | GTGACGCTGGCCATCTGCTCCTCCACGACGGCCTCCAGGCACCACCCCACCACC |
| | CTGGCCGTGGGAATCTGCTCTCCCGCCTCCCGTAGCATCAGCCTGCTGCGTCTC |
| | AGCTTCCACCAAGGTTGTACCATTGCCTCCCAGCGCCTGACGCCCCTGGCCCGA |
| | GGGGACACACTCTGCACCAACCTCACTGGGACACTTTTGCCTTCCCGAAACACT |
| | GATGAGACCTTCTTTGGAGTGCAGTGGGTGCGCCCCGGTGGCGGTGGCTCCGGA |
| | GGTGGCGGTTCCCTTGGGTGGGACGTAGCTGAGCTGCAGCTGAATCACACAGG |
| | ACCTCAGCAGGACCCCAGGCTATACTGGCAGGGGGGCCCAGCACTGGGCCGCT |
| | CCTTCCTGCATGGACCAGAGCTGGACAAGGGGCAGCTACGTATCCATCGTGATG |
| | GCATCTACATGGTACACATCCAGGTGACGCTGGCCATCTGCTCCTCCACGACGG |
| | CCTCCAGGCACCACCCCACCACCCTGGCCGTGGGAATCTGCTCTCCCGCCTCCC |
| | GTAGCATCAGCCTGCTGCGTCTCAGCTTCCACCAAGGTTGTACCATTGCCTCCC |
| | AGCGCCTGACGCCCCTGGCCCGAGGGGACACACTCTGCACCAACCTCACTGGG |
| | ACACTTTTGCCTTCCCGAAACACTGATGAGACCTTCTTTGGAGTGCAGTGGGTG |
| | CGCCCCGGTGGCGGTGGCTCCGGTGGCGGTGGCTCCCTTGGGTGGGACGTAGCT |
| | GAGCTGCAGCTGAATCACACAGGACCTCAGCAGGACCCCAGGCTATACTGGCA |
| | GGGGGGCCCAGCACTGGGCCGCTCCTTCCTGCATGGACCAGAGCTGGACAAGG |
| | GGCAGCTACGTATCCATCGTGATGGCATCTACATGGTACACATCCAGGTGACGC |
| | TGGCCATCTGCTCCTCCACGACGGCCTCCAGGCACCACCCCACCACCCTGGCCG |
| | TGGGAATCTGCTCTCCCGCCTCCCGTAGCATCAGCCTGCTGCGTCTCAGCTTCCA |
| | CCAAGGTTGTACCATTGCCTCCCAGCGCCTGACGCCCCTGGCCCGAGGGGACAC |
| | ACTCTGCACCAACCTCACTGGGACACTTTTGCCTTCCCGAAACACTGATGAGAC |
| | CTTCTTTGGAGTGCAGTGGGTGCGCCCCTGA (SEQ ID NO: 180) |
| | >Artificial sequence, M/P-4F2-Fc-scCD27L, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSLGWDVAELQLNHTGPQQDPRLYWQGGPALGRSF |
| | LHGPELDKGQLRIHRDGIYMVHIQVTLAICSSTTASRHHPTTLAVGICSPASRSISLLR |
| | LSFHQGCTIASQRLTPLARGDTLCTNLTGTLLPSRNTDETFFGVQWVRPGGGGSGG |
| | GGSLGWDVAELQLNHTGPQQDPRLYWQGGPALGRSFLHGPELDKGQLRIHRDGIY |
| | MVHIQVTLAICSSTTASRHHPTTLAVGICSPASRSISLLRLSFHQGCTIASQRLTPLAR |
| | GDTLCTNLTGTLLPSRNTDETFFGVQWVRPGGGGSGGGGSLGWDVAELQLNHTGP |
| | QQDPRLYWQGGPALGRSFLHGPELDKGQLRIHRDGIYMVHIQVTLAICSSTTASRH |
| | HPTTLAVGICSPASRSISLLRLSFHQGCTIASQRLTPLARGDTLCTNLTGTLLPSRNTD |
| | ETFFGVQWVRP (SEQ ID NO: 181) |
| Artificial | >Artificial sequence, Artificial Sequence M/P-4F2- |
| Sequence | Fc-scCD30L, mRNA, nucleic acid sequence |
| M/P-4F2-Fc- | ATGGGTTGCTGTTTCTCCAAGACCGGCTCGAGCGGCAGCCAGGACACCGAGGT |
| scCD30L | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCTGGGCCTACCTC |
| | CAAGTGGCAAAGCATCTAAACAAAACCAAGTTGTCTTGGAACAAAGATGGCAT |
| | TCTCCATGGAGTCAGATATCAGGATGGGAATCTGGTGATCCAATTCCCTGGTTT |
| | GTACTTCATCATTTGCCAACTGCAGTTTCTTGTACAATGCCCAAATAATTCTGTC |
| | GATCTGAAGTTGGAGCTTCTCATCAACAAGCATATCAAAAAACAGGCCCTGGTG |
| | ACAGTGTGTGAGTCTGGAATGCAAACGAAACACGTATACCAGAATCTCTCTCAA |
| | TTCTTGCTGGATTACCTGCAGGTCAACACCACCATATCAGTCAATGTGGATACA |
| | TTCCAGTACATAGATACAAGCACCTTTCCTCTTGAGAATGTGTTGTCCATCTTCT |
| | TATACAGTAATTCAGACGGTGGCGGTGGCTCCGGAGGTGGCGGTTCCTGGGCCT |
| | ACCTCCAAGTGGCAAAGCATCTAAACAAAACCAAGTTGTCTTGGAACAAAGAT |
| | GGCATTCTCCATGGAGTCAGATATCAGGATGGGAATCTGGTGATCCAATTCCCT |
| | GGTTTGTACTTCATCATTTGCCAACTGCAGTTTCTTGTACAATGCCCAAATAATT |
| | CTGTCGATCTGAAGTTGGAGCTTCTCATCAACAAGCATATCAAAAAACAGGCCC |
| | TGGTGACAGTGTGTGAGTCTGGAATGCAAACGAAACACGTATACCAGAATCTCT |
| | CTCAATTCTTGCTGGATTACCTGCAGGTCAACACCACCATATCAGTCAATGTGG |
| | ATACATTCCAGTACATAGATACAAGCACCTTTCCTCTTGAGAATGTGTTGTCCA |
| | TCTTCTTATACAGTAATTCAGACGGTGGCGGTGGCTCCGGTGGCGGTGGCTCCT |
| | GGGCCTACCTCCAAGTGGCAAAGCATCTAAACAAAACCAAGTTGTCTTGGAAC |
| | AAAGATGGCATTCTCCATGGAGTCAGATATCAGGATGGGAATCTGGTGATCCA |
| | ATTCCCTGGTTTGTACTTCATCATTTGCCAACTGCAGTTTCTTGTACAATGCCCA |
| | AATAATTCTGTCGATCTGAAGTTGGAGCTTCTCATCAACAAGCATATCAAAAAA |
| | CAGGCCCTGGTGACAGTGTGTGAGTCTGGAATGCAAACGAAACACGTATACCA |
| | GAATCTCTCTCAATTCTTGCTGGATTACCTGCAGGTCAACACCACCATATCAGT |
| | CAATGTGGATACATTCCAGTACATAGATACAAGCACCTTTCCTCTTGAGAATGT |
| | GTTGTCCATCTTCTTATACAGTAATTCAGACTGA (SEQ ID NO: 182) |
| | >Artificial sequence, M/P-4F2-Fc-scCD30L, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSWAYLQVAKHLNKTKLSWNKDGILHGVRYQDGN |
| | LVIQFPGLYFIICQLQFLVQCPNNSVDLKLELLINKHIKKQALVTVCESGMQTKHVY |
| | QNLSQFLLDYLQVNTTISVNVDTFQYIDTSTFPLENVLSIFLYSNSDGGGGSGGGGS |
| | WAYLQVAKHLNKTKLSWNKDGILHGVRYQDGNLVIQFPGLYFIICQLQFLVQCPN |
| | NSVDLKLELLINKHIKKQALVTVCESGMQTKHVYQNLSQFLLDYLQVNTTISVNVD |
| | TFQYIDTSTFPLENVLSIFLYSNSDGGGGSGGGGSWAYLQVAKHLNKTKLSWNKD |
| | GILHGVRYQDGNLVIQFPGLYFIICQLQFLVQCPNNSVDLKLELLINKHIKKQALVT |
| | VCESGMQTKHVYQNLSQFLLDYLQVNTTISVNVDTFQYIDTSTFPLENVLSIFLYSN |
| | SD |
| | (SEQ ID NO: 183) |
| Artificial | >Artificial sequence, M/P-4F2-Fc-scCD40L, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scCD40L | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGATCAGAATCCT |
| | CAAATTGCGGCACATGTCATAAGTGAGGCCAGCAGTAAAACAACATCTGTGTT |
| | ACAGTGGGCTGAAAAAGGATACTACACCATGAGCAACAACTTGGTAACCCTGG |
| | AAAATGGGAAACAGCTGACCGTTAAAAGACAAGGACTCTATTATATCTATGCC |
| | CAAGTCACCTTCTGTTCCAATCGGGAAGCTTCGAGTCAAGCTCCATTTATAGCC |
| | AGCCTCTGCCTAAAGTCCCCCGGTAGATTCGAGAGAATCTTACTCAGAGCTGCA |
| | AATACCCACAGTTCCGCCAAACCTTGCGGGCAACAATCCATTCACTTGGGAGGA |
| | GTATTTGAATTGCAACCAGGTGCTTCGGTGTTTGTCAATGTGACTGATCCAAGC |
| | CAAGTGAGCCATGGCACTGGCTTCACGTCCTTTGGCTTACTCAAACTCGGTGGC |
| | GGTGGCTCCGGAGGTGGCGGTTCCGATCAGAATCCTCAAATTGCGGCACATGTC |
| | ATAAGTGAGGCCAGCAGTAAAACAACATCTGTGTTACAGTGGGCTGAAAAAGG |
| | ATACTACACCATGAGCAACAACTTGGTAACCCTGGAAAATGGGAAACAGCTGA |
| | CCGTTAAAAGACAAGGACTCTATTATATCTATGCCCAAGTCACCTTCTGTTCCA |
| | ATCGGGAAGCTTCGAGTCAAGCTCCATTTATAGCCAGCCTCTGCCTAAAGTCCC |
| | CCGGTAGATTCGAGAGAATCTTACTCAGAGCTGCAAATACCCACAGTTCCGCCA |
| | AACCTTGCGGGCAACAATCCATTCACTTGGGAGGAGTATTTGAATTGCAACCAG |
| | GTGCTTCGGTGTTTGTCAATGTGACTGATCCAAGCCAAGTGAGCCATGGCACTG |
| | GCTTCACGTCCTTTGGCTTACTCAAACTCGGTGGCGGTGGCTCCGGTGGCGGTG |
| | GCTCCGATCAGAATCCTCAAATTGCGGCACATGTCATAAGTGAGGCCAGCAGTA |
| | AAACAACATCTGTGTTACAGTGGGCTGAAAAAGGATACTACACCATGAGCAAC |
| | AACTTGGTAACCCTGGAAAATGGGAAACAGCTGACCGTTAAAAGACAAGGACT |
| | CTATTATATCTATGCCCAAGTCACCTTCTGTTCCAATCGGGAAGCTTCGAGTCA |
| | AGCTCCATTTATAGCCAGCCTCTGCCTAAAGTCCCCCGGTAGATTCGAGAGAAT |
| | CTTACTCAGAGCTGCAAATACCCACAGTTCCGCCAAACCTTGCGGGCAACAATC |
| | CATTCACTTGGGAGGAGTATTTGAATTGCAACCAGGTGCTTCGGTGTTTGTCAA |
| | TGTGACTGATCCAAGCCAAGTGAGCCATGGCACTGGCTTCACGTCCTTTGGCTT |
| | ACTCAAACTCTGA |
| | (SEQ ID NO: 184) |
| | >Artificial sequence, M/P-4F2-Fc-scCD40L, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSDQNPQIAAHVISEASSKTTSVLQWAEKGYYTMSN |
| | NLVTLENGKQLTVKRQGLYYIYAQVTFCSNREASSQAPFIASLCLKSPGRFERILLR |
| | AANTHSSAKPCGQQSIHLGGVFELQPGASVFVNVTDPSQVSHGTGFTSFGLLKLGG |
| | GGSGGGGSDQNPQIAAHVISEASSKTTSVLQWAEKGYYTMSNNLVTLENGKQLTV |
| | KRQGLYYIYAQVTFCSNREASSQAPFIASLCLKSPGRFERILLRAANTHSSAKPCGQ |
| | QSIHLGGVFELQPGASVFVNVTDPSQVSHGTGFTSFGLLKLGGGGSGGGGSDQNPQ |
| | IAAHVISEASSKTTSVLQWAEKGYYTMSNNLVTLENGKQLTVKRQGLYYIYAQVT |
| | FCSNREASSQAPFIASLCLKSPGRFERILLRAANTHSSAKPCGQQSIHLGGVFELQPG |
| | ASVFVNVTDPSQVSHGTGFTSFGLLKL (SEQ ID NO: 185) |
| Artificial | >Artificial sequence, M/P-4F2-Fc-scLIGHT, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scLIGHT | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGTCAACCCAGC |
| | AGCGCATCTCACAGGGGCCAACTCCAGCTTGACCGGCAGCGGGGGGCCGCTGT |
| | TATGGGAGACTCAGCTGGGCCTGGCCTTCCTGAGGGGCCTCAGCTACCACGATG |
| | GGGCCCTTGTGGTCACCAAAGCTGGCTACTACTACATCTACTCCAAGGTGCAGC |
| | TGGGCGGTGTGGGCTGCCCGCTGGGCCTGGCCAGCACCATCACCCACGGCCTCT |
| | ACAAGCGCACACCCCGCTACCCCGAGGAGCTGGAGCTGTTGGTCAGCCAGCAG |
| | TCACCCTGCGGACGGGCCACCAGCAGCTCCCGGGTCTGGTGGGACAGCAGCTTC |
| | CTGGGTGGTGTGGTACACCTGGAGGCTGGGGAGAAGGTGGTCGTCCGTGTGCT |
| | GGATGAACGCCTGGTTCGACTGCGTGATGGTACCCGGTCTTACTTCGGGGCTTT |
| | CATGGTGGGTGGCGGTGGCTCCGTCAACCCAGCAGCGCATCTCACAGGGGCCA |
| | ACTCCAGCTTGACCGGCAGCGGGGGGCCGCTGTTATGGGAGACTCAGCTGGGC |
| | CTGGCCTTCCTGAGGGGCCTCAGCTACCACGATGGGGCCCTTGTGGTCACCAAA |
| | GCTGGCTACTACTACATCTACTCCAAGGTGCAGCTGGGCGGTGTGGGCTGCCCG |
| | CTGGGCCTGGCCAGCACCATCACCCACGGCCTCTACAAGCGCACACCCCGCTAC |
| | CCCGAGGAGCTGGAGCTGTTGGTCAGCCAGCAGTCACCCTGCGGACGGGCCAC |
| | CAGCAGCTCCCGGGTCTGGTGGGACAGCAGCTTCCTGGGTGGTGTGGTACACCT |
| | GGAGGCTGGGGAGAAGGTGGTCGTCCGTGTGCTGGATGAACGCCTGGTTCGAC |
| | TGCGTGATGGTACCCGGTCTTACTTCGGGGCTTTCATGGTGGGAGGTGGCGGCT |
| | CCGTCAACCCAGCAGCGCATCTCACAGGGGCCAACTCCAGCTTGACCGGCAGC |
| | GGGGGGCCGCTGTTATGGGAGACTCAGCTGGGCCTGGCCTTCCTGAGGGGCCTC |
| | AGCTACCACGATGGGGCCCTTGTGGTCACCAAAGCTGGCTACTACTACATCTAC |
| | TCCAAGGTGCAGCTGGGCGGTGTGGGCTGCCCGCTGGGCCTGGCCAGCACCATC |
| | ACCCACGGCCTCTACAAGCGCACACCCCGCTACCCCGAGGAGCTGGAGCTGTTG |
| | GTCAGCCAGCAGTCACCCTGCGGACGGGCCACCAGCAGCTCCCGGGTCTGGTG |
| | GGACAGCAGCTTCCTGGGTGGTGTGGTACACCTGGAGGCTGGGGAGAAGGTGG |
| | TCGTCCGTGTGCTGGATGAACGCCTGGTTCGACTGCGTGATGGTACCCGGTCTT |
| | ACTTCGGGGCTTTCATGGTGTGA |
| | (SEQ ID NO: 186) |
| | >Artificial sequence, M/P-4F2-Fc-scLIGHT, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSVNPAAHLTGANSSLTGSGGPLLWETQLGLAFLRG |
| | LSYHDGALVVTKAGYYYIYSKVQLGGVGCPLGLASTITHGLYKRTPRYPEELELLV |
| | SQQSPCGRATSSSRVWWDSSFLGGVVHLEAGEKVVVRVLDERLVRLRDGTRSYFG |
| | AFMVGGGGSVNPAAHLTGANSSLTGSGGPLLWETQLGLAFLRGLSYHDGALVVT |
| | KAGYYYIYSKVQLGGVGCPLGLASTITHGLYKRTPRYPEELELLVSQQSPCGRATSS |
| | SRVWWDSSFLGGVVHLEAGEKVVVRVLDERLVRLRDGTRSYFGAFMVGGGGSVN |
| | PAAHLTGANSSLTGSGGPLLWETQLGLAFLRGLSYHDGALVVTKAGYYYIYSKVQ |
| | LGGVGCPLGLASTITHGLYKRTPRYPEELELLVSQQSPCGRATSSSRVWWDSSFLG |
| | GVVHLEAGEKVVVRVLDERLVRLRDGTRSYFGAFMV (SEQ ID NO: 187) |
| Artificial | >Artificial sequence, M/P-4F2-Fc-scTRAIL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scTRAIL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCCAGAGAGTAGC |
| | AGCTCACATAACTGGGACCAGAGGAAGAAGCAACACATTGTCTTCTCCAAACT |
| | CCAAGAATGAAAAGGCTCTGGGCCGCAAAATAAACTCCTGGGAATCATCAAGG |
| | AGTGGGCATTCATTCCTGAGCAACTTGCACTTGAGGAATGGTGAACTGGTCATC |
| | CATGAAAAAGGGTTTTACTACATCTATTCCCAAACATACTTTCGATTTCAGGAG |
| | GAAATAAAAGAAAACACAAAGAACGACAAACAAATGGTCCAATATATTTACAA |
| | ATACACAAGTTATCCTGACCCTATATTGTTGATGAAAAGTGCTAGAAATAGTTG |
| | TTGGTCTAAAGATGCAGAATATGGACTCTATTCCATCTATCAAGGGGGAATATT |
| | TGAGCTTAAGGAAAATGACAGAATTTTTGTTTCTGTAACAAATGAGCACTTGAT |
| | AGACATGGACCATGAAGCCAGTTTTTTTGGGGCCTTTTTAGTTGGCGGTCAGAG |
| | AGTAGCAGCTCACATAACTGGGACCAGAGGAAGAAGCAACACATTGTCTTCTC |
| | CAAACTCCAAGAATGAAAAGGCTCTGGGCCGCAAAATAAACTCCTGGGAATCA |
| | TCAAGGAGTGGGCATTCATTCCTGAGCAACTTGCACTTGAGGAATGGTGAACTG |
| | GTCATCCATGAAAAAGGGTTTTACTACATCTATTCCCAAACATACTTTCGATTTC |
| | AGGAGGAAATAAAAGAAAACACAAAGAACGACAAACAAATGGTCCAATATAT |
| | TTACAAATACACAAGTTATCCTGACCCTATATTGTTGATGAAAAGTGCTAGAAA |
| | TAGTTGTTGGTCTAAAGATGCAGAATATGGACTCTATTCCATCTATCAAGGGGG |
| | AATATTTGAGCTTAAGGAAAATGACAGAATTTTTGTTTCTGTAACAAATGAGCA |
| | CTTGATAGACATGGACCATGAAGCCAGTTTTTTTGGGGCCTTTTTAGTTGGCGG |
| | CCAGAGAGTAGCAGCTCACATAACTGGGACCAGAGGAAGAAGCAACACATTGT |
| | CTTCTCCAAACTCCAAGAATGAAAAGGCTCTGGGCCGCAAAATAAACTCCTGG |
| | GAATCATCAAGGAGTGGGCATTCATTCCTGAGCAACTTGCACTTGAGGAATGGT |
| | GAACTGGTCATCCATGAAAAAGGGTTTTACTACATCTATTCCCAAACATACTTT |
| | CGATTTCAGGAGGAAATAAAAGAAAACACAAAGAACGACAAACAAATGGTCC |
| | AATATATTTACAAATACACAAGTTATCCTGACCCTATATTGTTGATGAAAAGTG |
| | CTAGAAATAGTTGTTGGTCTAAAGATGCAGAATATGGACTCTATTCCATCTATC |
| | AAGGGGGAATATTTGAGCTTAAGGAAAATGACAGAATTTTTGTTTCTGTAACAA |
| | ATGAGCACTTGATAGACATGGACCATGAAGCCAGTTTTTTTGGGGCCTTTTTAG |
| | TTGGCTAA (SEQ ID NO: 188) |
| | >Artificial sequence, M/P-4F2-Fc-scTRAIL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSQRVAAHITGTRGRSNTLSSPNSKNEKALGRKINSW |
| | ESSRSGHSFLSNLHLRNGELVIHEKGFYYIYSQTYFRFQEEIKENTKNDKQMVQYIY |
| | KYTSYPDPILLMKSARNSCWSKDAEYGLYSIYQGGIFELKENDRIFVSVTNEHLIDM |
| | DHEASFFGAFLVGGQRVAAHITGTRGRSNTLSSPNSKNEKALGRKINSWESSRSGH |
| | SFLSNLHLRNGELVIHEKGFYYIYSQTYFRFQEEIKENTKNDKQMVQYIYKYTSYPD |
| | PILLMKSARNSCWSKDAEYGLYSIYQGGIFELKENDRIFVSVTNEHLIDMDHEASFF |
| | GAFLVGGQRVAAHITGTRGRSNTLSSPNSKNEKALGRKINSWESSRSGHSFLSNLHL |
| | RNGELVIHEKGFYYIYSQTYFRFQEEIKENTKNDKQMVQYIYKYTSYPDPILLMKSA |
| | RNSCWSKDAEYGLYSIYQGGIFELKENDRIFVSVTNEHLIDMDHEASFFGAFLVG |
| | (SEQ ID NO: 189) |
| Artificial | >Artificial sequence, M/P-4F2-Fc-scRANKL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scRANKL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGAAGCTCAGCC |
| | TTTTGCTCATCTCACTATTAATGCCACCGACATCCCATCTGGTTCCCATAAAGTG |
| | AGTCTGTCCTCTTGGTACCATGATCGGGGTTGGGCCAAGATCTCCAACATGACT |
| | TTTAGCAATGGAAAACTAATAGTTAATCAGGATGGCTTTTATTACCTGTATGCC |
| | AACATTTGCTTTCGACATCATGAAACTTCAGGAGACCTAGCTACAGAGTATCTT |
| | CAACTAATGGTGTACGTCACTAAAACCAGCATCAAAATCCCAAGTTCTCATACC |
| | CTGATGAAAGGAGGAAGCACCAAGTATTGGTCAGGGAATTCTGAATTCCATTTT |
| | TATTCCATAAACGTTGGTGGATTTTTTAAGTTACGGTCTGGAGAGGAAATCAGC |
| | ATCGAGGTCTCCAACCCCTCCTTACTGGATCCGGATCAGGATGCAACATACTTT |
| | GGGGCTTTTAAAGTTCGAGATATAGATGGTGGCGGTGGCTCCGGAGGTGGCGG |
| | TTCCGAAGCTCAGCCTTTTGCTCATCTCACTATTAATGCCACCGACATCCCATCT |
| | GGTTCCCATAAAGTGAGTCTGTCCTCTTGGTACCATGATCGGGGTTGGGCCAAG |
| | ATCTCCAACATGACTTTTAGCAATGGAAAACTAATAGTTAATCAGGATGGCTTT |
| | TATTACCTGTATGCCAACATTTGCTTTCGACATCATGAAACTTCAGGAGACCTA |
| | GCTACAGAGTATCTTCAACTAATGGTGTACGTCACTAAAACCAGCATCAAAATC |
| | CCAAGTTCTCATACCCTGATGAAAGGAGGAAGCACCAAGTATTGGTCAGGGAA |
| | TTCTGAATTCCATTTTTATTCCATAAACGTTGGTGGATTTTTTAAGTTACGGTCT |
| | GGAGAGGAAATCAGCATCGAGGTCTCCAACCCCTCCTTACTGGATCCGGATCAG |
| | GATGCAACATACTTTGGGGCTTTTAAAGTTCGAGATATAGATGGTGGCGGTGGC |
| | TCCGGTGGCGGTGGCTCCGAAGCTCAGCCTTTTGCTCATCTCACTATTAATGCC |
| | ACCGACATCCCATCTGGTTCCCATAAAGTGAGTCTGTCCTCTTGGTACCATGAT |
| | CGGGGTTGGGCCAAGATCTCCAACATGACTTTTAGCAATGGAAAACTAATAGTT |
| | AATCAGGATGGCTTTTATTACCTGTATGCCAACATTTGCTTTCGACATCATGAA |
| | ACTTCAGGAGACCTAGCTACAGAGTATCTTCAACTAATGGTGTACGTCACTAAA |
| | ACCAGCATCAAAATCCCAAGTTCTCATACCCTGATGAAAGGAGGAAGCACCAA |
| | GTATTGGTCAGGGAATTCTGAATTCCATTTTTATTCCATAAACGTTGGTGGATTT |
| | TTTAAGTTACGGTCTGGAGAGGAAATCAGCATCGAGGTCTCCAACCCCTCCTTA |
| | CTGGATCCGGATCAGGATGCAACATACTTTGGGGCTTTTAAAGTTCGAGATATA |
| | GATTGA (SEQ ID NO: 190) |
| | >Artificial sequence, M/P-4F2-Fc-scRANKL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSEAQPFAHLTINATDIPSGSHKVSLSSWYHDRGWA |
| | KISNMTFSNGKLIVNQDGFYYLYANICFRHHETSGDLATEYLQLMVYVTKTSIKIPS |
| | SHTLMKGGSTKYWSGNSEFHFYSINVGGFFKLRSGEEISIEVSNPSLLDPDQDATYF |
| | GAFKVRDIDGGGGSGGGGSEAQPFAHLTINATDIPSGSHKVSLSSWYHDRGWAKIS |
| | NMTFSNGKLIVNQDGFYYLYANICFRHHETSGDLATEYLQLMVYVTKTSIKIPSSHT |
| | LMKGGSTKYWSGNSEFHFYSINVGGFFKLRSGEEISIEVSNPSLLDPDQDATYFGAF |
| | KVRDIDGGGGSGGGGSEAQPFAHLTINATDIPSGSHKVSLSSWYHDRGWAKISNMT |
| | FSNGKLIVNQDGFYYLYANICFRHHETSGDLATEYLQLMVYVTKTSIKIPSSHTLMK |
| | GGSTKYWSGNSEFHFYSINVGGFFKLRSGEEISIEVSNPSLLDPDQDATYFGAFKVR |
| | DID (SEQ ID NO: 191) |
| Artificial | >Artificial sequence, M/P-4F2-Fc-scTLIA, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scTL1A | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGATAAGCCAAG |
| | GGCACACCTGACAGTIGTGAGACAAACTCCCACACAGCACTTTAAAAATCAGTT |
| | CCCAGCTCTGCACTGGGAACATGAACTAGGCCTGGCCTTCACCAAGAACCGAAT |
| | GAACTATACCAACAAATTCCTGCTGATCCCAGAGTCGGGAGACTACTTCATTTA |
| | CTCCCAGGTCACATTCCGTGGGATGACCTCTGAGTGCAGTGAAATCAGACAAGC |
| | AGGCCGACCAAACAAGCCAGACTCCATCACTGTGGTCATCACCAAGGTAACAG |
| | ACAGCTACCCTGAGCCAACCCAGCTCCTCATGGGGACCAAGTCTGTATGCGAAG |
| | TAGGTAGCAACTGGTTCCAGCCCATCTACCTCGGAGCCATGTTCTCCTTGCAAG |
| | AAGGGGACAAGCTAATGGTGAACGTCAGTGACATCTCTTTGGTGGATTACACA |
| | AAAGAAGATAAAACCTTCTTTGGAGCCTTCTTACTAGGTGGCGGTGGCTCCGGA |
| | GGTGGCGGTTCCGATAAGCCAAGGGCACACCTGACAGTTGTGAGACAAACTCC |
| | CACACAGCACTTTAAAAATCAGTTCCCAGCTCTGCACTGGGAACATGAACTAGG |
| | CCTGGCCTTCACCAAGAACCGAATGAACTATACCAACAAATTCCTGCTGATCCC |
| | AGAGTCGGGAGACTACTTCATTTACTCCCAGGTCACATTCCGTGGGATGACCTC |
| | TGAGTGCAGTGAAATCAGACAAGCAGGCCGACCAAACAAGCCAGACTCCATCA |
| | CTGTGGTCATCACCAAGGTAACAGACAGCTACCCTGAGCCAACCCAGCTCCTCA |
| | TGGGGACCAAGTCTGTATGCGAAGTAGGTAGCAACTGGTTCCAGCCCATCTACC |
| | TCGGAGCCATGTTCTCCTTGCAAGAAGGGGACAAGCTAATGGTGAACGTCAGT |
| | GACATCTCTTTGGTGGATTACACAAAAGAAGATAAAACCTTCTTTGGAGCCTTC |
| | TTACTAGGTGGCGGTGGCTCCGGTGGCGGTGGCTCCGATAAGCCAAGGGCACA |
| | CCTGACAGTTGTGAGACAAACTCCCACACAGCACTTTAAAAATCAGTTCCCAGC |
| | TCTGCACTGGGAACATGAACTAGGCCTGGCCTTCACCAAGAACCGAATGAACT |
| | ATACCAACAAATTCCTGCTGATCCCAGAGTCGGGAGACTACTTCATTTACTCCC |
| | AGGTCACATTCCGTGGGATGACCTCTGAGTGCAGTGAAATCAGACAAGCAGGC |
| | CGACCAAACAAGCCAGACTCCATCACTGTGGTCATCACCAAGGTAACAGACAG |
| | CTACCCTGAGCCAACCCAGCTCCTCATGGGGACCAAGTCTGTATGCGAAGTAGG |
| | TAGCAACTGGTTCCAGCCCATCTACCTCGGAGCCATGTTCTCCTTGCAAGAAGG |
| | GGACAAGCTAATGGTGAACGTCAGTGACATCTCTTTGGTGGATTACACAAAAG |
| | AAGATAAAACCTTCTTTGGAGCCTTCTTACTATAG (SEQ ID NO: 192) |
| | >Artificial sequence, M/P-4F2-Fc-scTL1A, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSDKPRAHLTVVRQTPTQHFKNQFPALHWEHELGLA |
| | FTKNRMNYTNKFLLIPESGDYFIYSQVTFRGMTSECSEIRQAGRPNKPDSITVVITKV |
| | TDSYPEPTQLLMGTKSVCEVGSNWFQPIYLGAMFSLQEGDKLMVNVSDISLVDYT |
| | KEDKTFFGAFLLGGGGSGGGGSDKPRAHLTVVRQTPTQHFKNQFPALHWEHELGL |
| | AFTKNRMNYTNKFLLIPESGDYFIYSQVTFRGMTSECSEIRQAGRPNKPDSITVVITK |
| | VTDSYPEPTQLLMGTKSVCEVGSNWFQPIYLGAMFSLQEGDKLMVNVSDISLVDY |
| | TKEDKTFFGAFLLGGGGSGGGGSDKPRAHLTVVRQTPTQHFKNQFPALHWEHELG |
| | LAFTKNRMNYTNKFLLIPESGDYFIYSQVTFRGMTSECSEIRQAGRPNKPDSITVVIT |
| | KVTDSYPEPTQLLMGTKSVCEVGSNWFQPIYLGAMFSLQEGDKLMVNVSDISLVD |
| | YTKEDKTFFGAFLL (SEQ ID NO: 193) |
| Artificial | >Artificial sequence, M/P-4F2-Fc-scFASL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scFASL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCAGGAAAGTGGC |
| | CCATTTAACAGGCAAGTCCAACTCAAGGTCCATGCCTCTGGAATGGGAAGACA |
| | CCTATGGAATTGTCCTGCTTTCTGGAGTGAAGTATAAGAAGGGTGGCCTTGTGA |
| | TCAATGAAACTGGGCTGTACTTTGTATATTCCAAAGTATACTTCCGGGGTCAAT |
| | CTTGCAACAACCTGCCCCTGAGCCACAAGGTCTACATGAGGAACTCTAAGTATC |
| | CCCAGGATCTGGTGATGATGGAGGGGAAGATGATGAGCTACTGCACTACTGGG |
| | CAGATGTGGGCCCGCAGCAGCTACCTGGGGGCAGTGTTCAATCTTACCAGTGCT |
| | GATCATTTATATGTCAACGTATCTGAGCTCTCTCTGGTCAATTTTGAGGAATCTC |
| | AGACGTTTTTCGGCTTATATAAGCTCGGTGGCGGTGGCTCCGGAGGTGGCGGTT |
| | CCAGGAAAGTGGCCCATTTAACAGGCAAGTCCAACTCAAGGTCCATGCCTCTGG |
| | AATGGGAAGACACCTATGGAATTGTCCTGCTTTCTGGAGTGAAGTATAAGAAG |
| | GGTGGCCTTGTGATCAATGAAACTGGGCTGTACTTTGTATATTCCAAAGTATAC |
| | TTCCGGGGTCAATCTTGCAACAACCTGCCCCTGAGCCACAAGGTCTACATGAGG |
| | AACTCTAAGTATCCCCAGGATCTGGTGATGATGGAGGGGAAGATGATGAGCTA |
| | CTGCACTACTGGGCAGATGTGGGCCCGCAGCAGCTACCTGGGGGCAGTGTTCA |
| | ATCTTACCAGTGCTGATCATTTATATGTCAACGTATCTGAGCTCTCTCTGGTCAA |
| | TTTTGAGGAATCTCAGACGTTTTTCGGCTTATATAAGCTCGGTGGCGGTGGCTC |
| | CGGTGGCGGTGGCTCCAGGAAAGTGGCCCATTTAACAGGCAAGTCCAACTCAA |
| | GGTCCATGCCTCTGGAATGGGAAGACACCTATGGAATTGTCCTGCTTTCTGGAG |
| | TGAAGTATAAGAAGGGTGGCCTTGTGATCAATGAAACTGGGCTGTACTTTGTAT |
| | ATTCCAAAGTATACTTCCGGGGTCAATCTTGCAACAACCTGCCCCTGAGCCACA |
| | AGGTCTACATGAGGAACTCTAAGTATCCCCAGGATCTGGTGATGATGGAGGGG |
| | AAGATGATGAGCTACTGCACTACTGGGCAGATGTGGGCCCGCAGCAGCTACCT |
| | GGGGGCAGTGTTCAATCTTACCAGTGCTGATCATTTATATGTCAACGTATCTGA |
| | GCTCTCTCTGGTCAATTTTGAGGAATCTCAGACGTTTTTCGGCTTATATAAGCTC |
| | TAA (SEQ ID NO: 194) |
| | >Artificial sequence, M/P-4F2-Fc-scFASL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSRKVAHLTGKSNSRSMPLEWEDTYGIVLLSGVKYK |
| | KGGLVINETGLYFVYSKVYFRGQSCNNLPLSHKVYMRNSKYPQDLVMMEGKMMS |
| | YCTTGQMWARSSYLGAVFNLTSADHLYVNVSELSLVNFEESQTFFGLYKLGGGGS |
| | GGGGSRKVAHLTGKSNSRSMPLEWEDTYGIVLLSGVKYKKGGLVINETGLYFVYS |
| | KVYFRGQSCNNLPLSHKVYMRNSKYPQDLVMMEGKMMSYCTTGQMWARSSYLG |
| | AVFNLTSADHLYVNVSELSLVNFEESQTFFGLYKLGGGGSGGGGSRKVAHLTGKS |
| | NSRSMPLEWEDTYGIVLLSGVKYKKGGLVINETGLYFVYSKVYFRGQSCNNLPLSH |
| | KVYMRNSKYPQDLVMMEGKMMSYCTTGQMWARSSYLGAVFNLTSADHLYVNV |
| | SELSLVNFEESQTFFGLYKL (SEQ ID NO: 195) |
| Artificial | >Artificial sequence, M/P-4F2-Fc-scBAFF, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scBAFF | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGAAACAGTCAC |
| | TCAAGACTGCTTGCAACTGATTGCAGACAGTGAAACACCAACTATACAAAAAG |
| | GATCTTACACATTTGTTCCATGGCTTCTCAGCTTTAAAAGGGGAAGTGCCCTAG |
| | AAGAAAAAGAGAATAAAATATTGGTCAAAGAAACTGGTTACTTTTTTATATATG |
| | GTCAGGTTTTATATACTGATAAGACCTACGCCATGGGACATCTAATTCAGAGGA |
| | AGAAGGTCCATGTCTTTGGGGATGAATTGAGTCTGGTGACTTTGTTTCGATGTA |
| | TTCAAAATATGCCTGAAACACTACCCAATAATTCCTGCTATTCAGCTGGCATTG |
| | CAAAACTGGAAGAAGGAGATGAACTCCAACTTGCAATACCAAGAGAAAATGCA |
| | CAAATATCACTGGATGGAGATGTCACATTTTTTGGTGCATTGAAACTGCTGGGT |
| | GGCGGTGGCTCCGGAGGTGGCGGTTCCGAAACAGTCACTCAAGACTGCTTGCA |
| | ACTGATTGCAGACAGTGAAACACCAACTATACAAAAAGGATCTTACACATTTGT |
| | TCCATGGCTTCTCAGCTTTAAAAGGGGAAGTGCCCTAGAAGAAAAAGAGAATA |
| | AAATATTGGTCAAAGAAACTGGTTACTTTTTTATATATGGTCAGGTTTTATATAC |
| | TGATAAGACCTACGCCATGGGACATCTAATTCAGAGGAAGAAGGTCCATGTCTT |
| | TGGGGATGAATTGAGTCTGGTGACTTTGTTTCGATGTATTCAAAATATGCCTGA |
| | AACACTACCCAATAATTCCTGCTATTCAGCTGGCATTGCAAAACTGGAAGAAGG |
| | AGATGAACTCCAACTTGCAATACCAAGAGAAAATGCACAAATATCACTGGATG |
| | GAGATGTCACATTTTTTGGTGCATTGAAACTGCTGGGTGGCGGTGGCTCCGGTG |
| | GCGGTGGCTCCGAAACAGTCACTCAAGACTGCTTGCAACTGATTGCAGACAGTG |
| | AAACACCAACTATACAAAAAGGATCTTACACATTTGTTCCATGGCTTCTCAGCT |
| | TTAAAAGGGGAAGTGCCCTAGAAGAAAAAGAGAATAAAATATTGGTCAAAGA |
| | AACTGGTTACTTTTTTATATATGGTCAGGTTTTATATACTGATAAGACCTACGCC |
| | ATGGGACATCTAATTCAGAGGAAGAAGGTCCATGTCTTTGGGGATGAATTGAGT |
| | CTGGTGACTTTGTTTCGATGTATTCAAAATATGCCTGAAACACTACCCAATAAT |
| | TCCTGCTATTCAGCTGGCATTGCAAAACTGGAAGAAGGAGATGAACTCCAACTT |
| | GCAATACCAAGAGAAAATGCACAAATATCACTGGATGGAGATGTCACATTTTTT |
| | GGTGCATTGAAACTGCTGTGA |
| | (SEQ ID NO: 196) |
| | >Artificial sequence, M/P-4F2-Fc-scBAFF, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSETVTQDCLQLIADSETPTIQKGSYTFVPWLLSFKR |
| | GSALEEKENKILVKETGYFFIYGQVLYTDKTYAMGHLIQRKKVHVFGDELSLVTLF |
| | RCIQNMPETLPNNSCYSAGIAKLEEGDELQLAIPRENAQISLDGDVTFFGALKLLGG |
| | GGSGGGGSETVTQDCLQLIADSETPTIQKGSYTFVPWLLSFKRGSALEEKENKILVK |
| | ETGYFFIYGQVLYTDKTYAMGHLIQRKKVHVFGDELSLVTLFRCIQNMPETLPNNS |
| | CYSAGIAKLEEGDELQLAIPRENAQISLDGDVTFFGALKLLGGGGSGGGGSETVTQ |
| | DCLQLIADSETPTIQKGSYTFVPWLLSFKRGSALEEKENKILVKETGYFFIYGQVLYT |
| | DKTYAMGHLIQRKKVHVFGDELSLVTLFRCIQNMPETLPNNSCYSAGIAKLEEGDE |
| | LQLAIPRENAQISLDGDVTFFGALKLL (SEQ ID NO: 197) |
| Artificial | >Artificial sequence, M/P-4F2-Fc-scAPRIL, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| ScAPRIL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCCAGAAGAAGCA |
| | GCACTCTGTCCTGCACCTGGTTCCCATTAACGCCACCTCCAAGGATGACTCCGA |
| | TGTGACAGAGGTGATGTGGCAACCAGCTCTTAGGCGTGGGAGAGGCCTACAGG |
| | CCCAAGGATATGGTGTCCGAATCCAGGATGCTGGAGTTTATCTGCTGTATAGCC |
| | AGGTCCTGTTTCAAGACGTGACTTTCACCATGGGTCAGGTGGTGTCTCGAGAAG |
| | GCCAAGGAAGGCAGGAGACTCTATTCCGATGTATAAGAAGTATGCCCTCCCAC |
| | CCGGACCGGGCCTACAACAGCTGCTATAGCGCAGGTGTCTTCCATTTACACCAA |
| | GGGGATATTCTGAGTGTCATAATTCCCCGGGCAAGGGCGAAACTTAACCTCTCT |
| | CCACATGGAACCTTCCTGGGGTTTGTGAAACTGGGTGGCGGTGGCTCCGGAGGT |
| | GGCGGTTCCCAGAAGAAGCAGCACTCTGTCCTGCACCTGGTTCCCATTAACGCC |
| | ACCTCCAAGGATGACTCCGATGTGACAGAGGTGATGTGGCAACCAGCTCTTAG |
| | GCGTGGGAGAGGCCTACAGGCCCAAGGATATGGTGTCCGAATCCAGGATGCTG |
| | GAGTTTATCTGCTGTATAGCCAGGTCCTGTTTCAAGACGTGACTTTCACCATGG |
| | GTCAGGTGGTGTCTCGAGAAGGCCAAGGAAGGCAGGAGACTCTATTCCGATGT |
| | ATAAGAAGTATGCCCTCCCACCCGGACCGGGCCTACAACAGCTGCTATAGCGC |
| | AGGTGTCTTCCATTTACACCAAGGGGATATTCTGAGTGTCATAATTCCCCGGGC |
| | AAGGGCGAAACTTAACCTCTCTCCACATGGAACCTTCCTGGGGTTTGTGAAACT |
| | GGGTGGCGGTGGCTCCGGTGGCGGTGGCTCCCAGAAGAAGCAGCACTCTGTCC |
| | TGCACCTGGTTCCCATTAACGCCACCTCCAAGGATGACTCCGATGTGACAGAGG |
| | TGATGTGGCAACCAGCTCTTAGGCGTGGGAGAGGCCTACAGGCCCAAGGATAT |
| | GGTGTCCGAATCCAGGATGCTGGAGTTTATCTGCTGTATAGCCAGGTCCTGTTT |
| | CAAGACGTGACTTTCACCATGGGTCAGGTGGTGTCTCGAGAAGGCCAAGGAAG |
| | GCAGGAGACTCTATTCCGATGTATAAGAAGTATGCCCTCCCACCCGGACCGGGC |
| | CTACAACAGCTGCTATAGCGCAGGTGTCTTCCATTTACACCAAGGGGATATTCT |
| | GAGTGTCATAATTCCCCGGGCAAGGGCGAAACTTAACCTCTCTCCACATGGAAC |
| | CTTCCTGGGGTTTGTGAAACTGTGA (SEQ ID NO: 198) |
| | >Artificial sequence, M/P-4F2-Fc-scAPRIL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGGGGGSGGGGSQKKQHSVLHLVPINATSKDDSDVTEVMWQPALR |
| | RGRGLQAQGYGVRIQDAGVYLLYSQVLFQDVTFTMGQVVSREGQGRQETLFRCIR |
| | SMPSHPDRAYNSCYSAGVFHLHQGDILSVIIPRARAKLNLSPHGTFLGFVKLGGGGS |
| | GGGGSQKKQHSVLHLVPINATSKDDSDVTEVMWQPALRRGRGLQAQGYGVRIQD |
| | AGVYLLYSQVLFQDVTFTMGQVVSREGQGRQETLFRCIRSMPSHPDRAYNSCYSA |
| | GVFHLHQGDILSVIIPRARAKLNLSPHGTFLGFVKLGGGGSGGGGSQKKQHSVLHL |
| | VPINATSKDDSDVTEVMWQPALRRGRGLQAQGYGVRIQDAGVYLLYSQVLFQDV |
| | TFTMGQVVSREGQGRQETLFRCIRSMPSHPDRAYNSCYSAGVFHLHQGDILSVIIPR |
| | ARAKLNLSPHGTFLGFVKL (SEQ ID NO: 199) |
| Artificial | >Artificial sequence, M/P-4F2-Fc-scTWEAK, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| scTWEAK | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCGCACCTAAAGG |
| | CCGGAAAACACGGGCTCGAAGAGCGATCGCAGCCCATTATGAAGTTCATCCAC |
| | GACCTGGACAGGACGGAGCGCAGGCAGGTGTGGACGGGACAGTGAGTGGCTG |
| | GGAGGAAGCCAGAATCAACAGCTCCAGCCCTCTGCGCTACAACCGCCAGATCG |
| | GGGAGTTTATAGTCACCCGGGCTGGGCTCTACTACCTGTACTGTCAGGTGCACT |
| | TTGATGAGGGGAAGGCTGTCTACCTGAAGCTGGACTTGCTGGTGGATGGTGTGC |
| | TGGCCCTGCGCTGCCTGGAGGAATTCTCAGCCACTGCGGCGAGTTCCCTCGGGC |
| | CCCAGCTCCGCCTCTGCCAGGTGTCTGGGCTGTTGGCCCTGCGGCCAGGGTCCT |
| | CCCTGCGGATCCGCACCCTCCCCTGGGCCCATCTCAAGGCTGCCCCCTTCCTCA |
| | CCTACTTCGGACTCTTCCAGGTTCACGGTGGCGGTGGCTCCGGAGGTGGCGGTT |
| | CCGCACCTAAAGGCCGGAAAACACGGGCTCGAAGAGCGATCGCAGCCCATTAT |
| | GAAGTTCATCCACGACCTGGACAGGACGGAGCGCAGGCAGGTGTGGACGGGAC |
| | AGTGAGTGGCTGGGAGGAAGCCAGAATCAACAGCTCCAGCCCTCTGCGCTACA |
| | ACCGCCAGATCGGGGAGTTTATAGTCACCCGGGCTGGGCTCTACTACCTGTACT |
| | GTCAGGTGCACTTTGATGAGGGGAAGGCTGTCTACCTGAAGCTGGACTTGCTGG |
| | TGGATGGTGTGCTGGCCCTGCGCTGCCTGGAGGAATTCTCAGCCACTGCGGCGA |
| | GTTCCCTCGGGCCCCAGCTCCGCCTCTGCCAGGTGTCTGGGCTGTTGGCCCTGC |
| | GGCCAGGGTCCTCCCTGCGGATCCGCACCCTCCCCTGGGCCCATCTCAAGGCTG |
| | CCCCCTTCCTCACCTACTTCGGACTCTTCCAGGTTCACGGTGGCGGTGGCTCCGG |
| | TGGCGGTGGCTCCGCACCTAAAGGCCGGAAAACACGGGCTCGAAGAGCGATCG |
| | CAGCCCATTATGAAGTTCATCCACGACCTGGACAGGACGGAGCGCAGGCAGGT |
| | GTGGACGGGACAGTGAGTGGCTGGGAGGAAGCCAGAATCAACAGCTCCAGCCC |
| | TCTGCGCTACAACCGCCAGATCGGGGAGTTTATAGTCACCCGGGCTGGGCTCTA |
| | CTACCTGTACTGTCAGGTGCACTTTGATGAGGGGAAGGCTGTCTACCTGAAGCT |
| | GGACTTGCTGGTGGATGGTGTGCTGGCCCTGCGCTGCCTGGAGGAATTCTCAGC |
| | CACTGCGGCGAGTTCCCTCGGGCCCCAGCTCCGCCTCTGCCAGGTGTCTGGGCT |
| | GTTGGCCCTGCGGCCAGGGTCCTCCCTGCGGATCCGCACCCTCCCCTGGGCCCA |
| | TCTCAAGGCTGCCCCCTTCCTCACCTACTTCGGACTCTTCCAGGTTCACTGA |
| | (SEQ ID NO: 200) |
| | >Artificial sequence, M/P-4F2-Fc-scTWEAK, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSAPKGRKTRARRAIAAHYEVHPRPGQDGAQAGVD |
| | GTVSGWEEARINSSSPLRYNRQIGEFIVTRAGLYYLYCQVHFDEGKAVYLKLDLLV |
| | DGVLALRCLEEFSATAASSLGPQLRLCQVSGLLALRPGSSLRIRTLPWAHLKAAPFL |
| | TYFGLFQVHGGGGSGGGGSAPKGRKTRARRAIAAHYEVHPRPGQDGAQAGVDGT |
| | VSGWEEARINSSSPLRYNRQIGEFIVTRAGLYYLYCQVHFDEGKAVYLKLDLLVDG |
| | VLALRCLEEFSATAASSLGPQLRLCQVSGLLALRPGSSLRIRTLPWAHLKAAPFLTY |
| | FGLFQVHGGGGSGGGGSAPKGRKTRARRAIAAHYEVHPRPGQDGAQAGVDGTVS |
| | GWEEARINSSSPLRYNRQIGEFIVTRAGLYYLYCQVHFDEGKAVYLKLDLLVDGVL |
| | ALRCLEEFSATAASSLGPQLRLCQVSGLLALRPGSSLRIRTLPWAHLKAAPFLTYFG |
| | LFQVH (SEQ ID NO: 201) |
| Artificial | >Artificial sequence, M/P-4F2-4-1BBL, mRNA, |
| Sequence | nucleic acid sequence |
| M/P-4F2-4- | ATGGGTTGCTGTTTCTCCAAGACCGGCTCGAGCGGCAGCCAGGACACCGAGGT |
| 1BBL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGGCCTGCCCCTGGGCCGTGTCCGGGGCT |
| | CGCGCCTCGCCCGGCTCCGCGGCCAGCCCGAGACTCCGCGAGGGTCCCGAGCTT |
| | TCGCCCGACGATCCCGCCGGCCTCTTGGACCTGCGGCAGGGCATGTTTGCGCAG |
| | CTGGTGGCCCAAAATGTTCTGCTGATCGATGGGCCCCTGAGCTGGTACAGTGAC |
| | CCAGGCCTGGCAGGCGTGTCCCTGACGGGGGGCCTGAGCTACAAAGAGGACAC |
| | GAAGGAGCTGGTGGTGGCCAAGGCTGGAGTCTACTATGTCTTCTTTCAACTAGA |
| | GCTGCGGCGCGTGGTGGCCGGCGAGGGCTCAGGCTCCGTTTCACTTGCGCTGCA |
| | CCTGCAGCCACTGCGCTCTGCTGCTGGGGCCGCCGCCCTGGCTTTGACCGTGGA |
| | CCTGCCACCCGCCTCCTCCGAGGCTCGGAACTCGGCCTTCGGTTTCCAGGGCCG |
| | CTTGCTGCACCTGAGTGCCGGCCAGCGCCTGGGCGTCCATCTTCACACTGAGGC |
| | CAGGGCACGCCATGCCTGGCAGCTTACCCAGGGCGCCACAGTCTTGGGACTCTT |
| | CCGGGTGACCCCCGAAATCCCAGCCGGACTCCCTTCACCGAGGTCGGAATAA |
| | (SEQ ID NO: 202) |
| | >Artificial sequence, M/P-4F2-4-1BBL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVACPWAVSGARASPGSAASPRLREGPELSPDDPAGLLDLRQGMFAQLVAQ |
| | NVLLIDGPLSWYSDPGLAGVSLTGGLSYKEDTKELVVAKAGVYYVFFQLELRRVV |
| | AGEGSGSVSLALHLQPLRSAAGAAALALTVDLPPASSEARNSAFGFQGRLLHLSAG |
| | QRLGVHLHTEARARHAWQLTQGATVLGLFRVTPEIPAGLPSPRSE |
| | (SEQ ID NO: 203) |
| Artificial | >Artificial sequence, M/P-CD298-sc4-1BBL, mRNA, |
| Sequence | nucleic acid sequence |
| M/P-CD298- | ATGGGTTGCTGTTTCTCCAAGACCGGCTCGAGCGGCACGAAGAACGAGAAGAA |
| sc4-1BBL | GTCCCTCAACCAGAGCCTGGCCGAGTGGAAGCTCTTCATCTACAACCCGACCAC |
| | CGGAGAATTCCTGGGGCGCACCGCCAAGAGCTGGGGTTTGATCTTGCTCTTCTA |
| | CCTAGTTTTTTATGGGTTCCTGGCTGCACTCTTCTCATTCACGATGTGGGTTATG |
| | CTTCAGACTCTCAACGATGAGGTTCCAAAATACCGTGACCAGATTCCTAGCCCA |
| | GGACTCATGGTTTTTCCAAAACCAGTGACCGCATTGGAATATACATTCAGTAGG |
| | TCTGATCCAACTTCGTATGCAGGGTACATTGAAGACCTTAAGAAGTTTCTAAAA |
| | CCATATACTTTAGAAGAACAGAAGAACCTCACAGTCTGTCCTGATGGAGCACTT |
| | TTTGAACAGAAGGGTCCAGTTTATGTTGCATGTCAGTTTCCTATTTCATTACTTC |
| | AAGCATGCAGTGGTATGAATGATCCTGATTTTGGCTATTCTCAAGGAAACCCTT |
| | GTATTCTTGTGAAAATGAACAGAATAATTGGATTAAAGCCTGAAGGAGTGCCA |
| | AGGATAGATTGTGTTTCAAAGAATGAAGATATACCAAATGTAGCAGTTTATCCT |
| | CATAATGGAATGATAGACTTAAAATATTTCCCATATTATGGGAAAAAACTGCAT |
| | GTTGGGTATCTACAGCCATTGGTTGCTGTTCAGGTCAGCTTTGCTCCTAACAAC |
| | ACTGGGAAAGAAGTAACAGTTGAGTGCAAGATTGATGGATCAGCCAACCTAAA |
| | AAGTCAGGATGATCGTGACAAGTTTTTGGGACGAGTTATGTTCAAAATCACAGC |
| | ACGTGCAGCCTGCCCCTGGGCCGTGTCCGGGGCTCGCGCCTCGCCCGGCTCCGC |
| | GGCCAGCCCGAGACTCCGCGAGGGTCCCGAGCTTTCGCCCGACGATCCCGCCG |
| | GCCTCTTGGACCTGCGGCAGGGCATGTTTGCGCAGCTGGTGGCCCAAAATGTTC |
| | TGCTGATCGATGGGCCCCTGAGCTGGTACAGTGACCCAGGCCTGGCAGGCGTGT |
| | CCCTGACGGGGGGCCTGAGCTACAAAGAGGACACGAAGGAGCTGGTGGTGGCC |
| | AAGGCTGGAGTCTACTATGTCTTCTTTCAACTAGAGCTGCGGCGCGTGGTGGCC |
| | GGCGAGGGCTCAGGCTCCGTTTCACTTGCGCTGCACCTGCAGCCACTGCGCTCT |
| | GCTGCTGGGGCCGCCGCCCTGGCTTTGACCGTGGACCTGCCACCCGCCTCCTCC |
| | GAGGCTCGGAACTCGGCCTTCGGTTTCCAGGGCCGCTTGCTGCACCTGAGTGCC |
| | GGCCAGCGCCTGGGCGTCCATCTTCACACTGAGGCCAGGGCACGCCATGCCTGG |
| | CAGCTTACCCAGGGCGCCACAGTCTTGGGACTCTTCCGGGTGACCCCCGAAATC |
| | CCAGCCGGACTCCCTTCACCGAGGTCGGAAggtggcggtggatccggtggcggtggatccggtggc |
| | ggtggatccggtggcggtggatccCGCGAGGGTCCCGAGCTTTCGCCCGACGATCCCGCCGG |
| | CCTCTTGGACCTGCGGCAGGGCATGTTTGCGCAGCTGGTGGCCCAAAATGTTCT |
| | GCTGATCGATGGGCCCCTGAGCTGGTACAGTGACCCAGGCCTGGCAGGCGTGTC |
| | CCTGACGGGGGGCCTGAGCTACAAAGAGGACACGAAGGAGCTGGTGGTGGCCA |
| | AGGCTGGAGTCTACTATGTCTTCTTTCAACTAGAGCTGCGGCGCGTGGTGGCCG |
| | GCGAGGGCTCAGGCTCCGTTTCACTTGCGCTGCACCTGCAGCCACTGCGCTCTG |
| | CTGCTGGGGCCGCCGCCCTGGCTTTGACCGTGGACCTGCCACCCGCCTCCTCCG |
| | AGGCTCGGAACTCGGCCTTCGGTTTCCAGGGCCGCTTGCTGCACCTGAGTGCCG |
| | GCCAGCGCCTGGGCGTCCATCTTCACACTGAGGCCAGGGCACGCCATGCCTGGC |
| | AGCTTACCCAGGGCGCCACAGTCTTGGGACTCTTCCGGGTGACCCCCGAAATCC |
| | CAGCCGGACTCCCTTCACCGAGGTCGGAAggtggcggtggatccggtggcggtggatccggtggcgg |
| | tggatccggtggcggtggatccCGCGAGGGTCCCGAGCTTTCGCCCGACGATCCCGCCGGCC |
| | TCTTGGACCTGCGGCAGGGCATGTTTGCGCAGCTGGTGGCCCAAAATGTTCTGC |
| | TGATCGATGGGCCCCTGAGCTGGTACAGTGACCCAGGCCTGGCAGGCGTGTCCC |
| | TGACGGGGGGCCTGAGCTACAAAGAGGACACGAAGGAGCTGGTGGTGGCCAA |
| | GGCTGGAGTCTACTATGTCTTCTTTCAACTAGAGCTGCGGCGCGTGGTGGCCGG |
| | CGAGGGCTCAGGCTCCGTTTCACTTGCGCTGCACCTGCAGCCACTGCGCTCTGC |
| | TGCTGGGGCCGCCGCCCTGGCTTTGACCGTGGACCTGCCACCCGCCTCCTCCGA |
| | GGCTCGGAACTCGGCCTTCGGTTTCCAGGGCCGCTTGCTGCACCTGAGTGCCGG |
| | CCAGCGCCTGGGCGTCCATCTTCACACTGAGGCCAGGGCACGCCATGCCTGGCA |
| | GCTTACCCAGGGCGCCACAGTCTTGGGACTCTTCCGGGTGACCCCCGAAATCCC |
| | AGCCGGACTCCCTTCACCGAGGTCGGAATAA (SEQ ID NO: 204) |
| | >Artificial sequence, M/P-CD298-sc4-1BBL, amino acid |
| | sequence |
| | MGCCFSKTGSSGTKNEKKSLNQSLAEWKLFIYNPTTGEFLGRTAKSWGLILLFYLV |
| | FYGFLAALFSFTMWVMLQTLNDEVPKYRDQIPSPGLMVFPKPVTALEYTFSRSDPT |
| | SYAGYIEDLKKFLKPYTLEEQKNLTVCPDGALFEQKGPVYVACQFPISLLQACSGM |
| | NDPDFGYSQGNPCILVKMNRIIGLKPEGVPRIDCVSKNEDIPNVAVYPHNGMIDLKY |
| | FPYYGKKLHVGYLQPLVAVQVSFAPNNTGKEVTVECKIDGSANLKSQDDRDKFLG |
| | RVMFKITARAACPWAVSGARASPGSAASPRLREGPELSPDDPAGLLDLRQGMFAQ |
| | LVAQNVLLIDGPLSWYSDPGLAGVSLTGGLSYKEDTKELVVAKAGVYYVFFQLEL |
| | RRVVAGEGSGSVSLALHLQPLRSAAGAAALALTVDLPPASSEARNSAFGFQGRLLH |
| | LSAGQRLGVHLHTEARARHAWQLTQGATVLGLFRVTPEIPAGLPSPRSEGGGGSGG |
| | GGSGGGGSGGGGSREGPELSPDDPAGLLDLRQGMFAQLVAQNVLLIDGPLSWYSD |
| | PGLAGVSLTGGLSYKEDTKELVVAKAGVYYVFFQLELRRVVAGEGSGSVSLALHL |
| | QPLRSAAGAAALALTVDLPPASSEARNSAFGFQGRLLHLSAGQRLGVHLHTEARA |
| | RHAWQLTQGATVLGLFRVTPEIPAGLPSPRSEGGGGSGGGGSGGGGSGGGGSREGP |
| | ELSPDDPAGLLDLRQGMFAQLVAQNVLLIDGPLSWYSDPGLAGVSLTGGLSYKED |
| | TKELVVAKAGVYYVFFQLELRRVVAGEGSGSVSLALHLQPLRSAAGAAALALTVD |
| | LPPASSEARNSAFGFQGRLLHLSAGQRLGVHLHTEARARHAWQLTQGATVLGLFR |
| | VTPEIPAGLPSPRSE (SEQ ID NO: 205) |
| Artificial | >Artificial sequence, M/P-4F2-sc4-1BBL, mRNA, |
| Sequence | nucleic acid sequence |
| M/P-4F2-sc4- | ATGGGTTGCTGTTTCTCCAAGACCGGCTCGAGCGGCAGCCAGGACACCGAGGT |
| 1BBL | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGAGGTGGCGGTGGCTCCGGTGGCGGT |
| | GGCTCCGGTGGCGGTGGATCCCGCGAGGGTCCCGAGCTTTCGCCCGACGATCCC |
| | GCCGGCCTCTTGGACCTGCGGCAGGGCATGTTTGCGCAGCTGGTGGCCCAAAAT |
| | GTTCTGCTGATCGATGGGCCCCTGAGCTGGTACAGTGACCCAGGCCTGGCAGGC |
| | GTGTCCCTGACGGGGGGCCTGAGCTACAAAGAGGACACGAAGGAGCTGGTGGT |
| | GGCCAAGGCTGGAGTCTACTATGTCTTCTTTCAACTAGAGCTGCGGCGCGTGGT |
| | GGCCGGCGAGGGCTCAGGCTCCGTTTCACTTGCGCTGCACCTGCAGCCACTGCG |
| | CTCTGCTGCTGGGGCCGCCGCCCTGGCTTTGACCGTGGACCTGCCACCCGCCTC |
| | CTCCGAGGCTCGGAACTCGGCCTTCGGTTTCCAGGGCCGCTTGCTGCACCTGAG |
| | TGCCGGCCAGCGCCTGGGCGTCCATCTTCACACTGAGGCCAGGGCACGCCATGC |
| | CTGGCAGCTTACCCAGGGCGCCACAGTCTTGGGACTCTTCCGGGTGACCCCCGA |
| | AATCCCAGCCGGACTCCCTTCACCGAGGTCGGAAGGTGGCGGTGGCTCCGGAG |
| | GTGGCGGTTCCGGTGGCGGTGGCTCCGGTGGCGGTGGCTCCCGCGAGGGTCCCG |
| | AGCTTTCGCCCGACGATCCCGCCGGCCTCTTGGACCTGCGGCAGGGCATGTTTG |
| | CGCAGCTGGTGGCCCAAAATGTTCTGCTGATCGATGGGCCCCTGAGCTGGTACA |
| | GTGACCCAGGCCTGGCAGGCGTGTCCCTGACGGGGGGCCTGAGCTACAAAGAG |
| | GACACGAAGGAGCTGGTGGTGGCCAAGGCTGGAGTCTACTATGTCTTCTTTCAA |
| | CTAGAGCTGCGGCGCGTGGTGGCCGGCGAGGGCTCAGGCTCCGTTTCACTTGCG |
| | CTGCACCTGCAGCCACTGCGCTCTGCTGCTGGGGCCGCCGCCCTGGCTTTGACC |
| | GTGGACCTGCCACCCGCCTCCTCCGAGGCTCGGAACTCGGCCTTCGGTTTCCAG |
| | GGCCGCTTGCTGCACCTGAGTGCCGGCCAGCGCCTGGGCGTCCATCTTCACACT |
| | GAGGCCAGGGCACGCCATGCCTGGCAGCTTACCCAGGGCGCCACAGTCTTGGG |
| | ACTCTTCCGGGTGACCCCCGAAATCCCAGCCGGACTCCCTTCACCGAGGTCGGA |
| | AGGTGGCGGTGGCTCCGGTGGCGGTGGCTCCGGTGGCGGTGGCTCCGGAGGTG |
| | GCGGCTCCCGCGAGGGTCCCGAGCTTTCGCCCGACGATCCCGCCGGCCTCTTGG |
| | ACCTGCGGCAGGGCATGTTTGCGCAGCTGGTGGCCCAAAATGTTCTGCTGATCG |
| | ATGGGCCCCTGAGCTGGTACAGTGACCCAGGCCTGGCAGGCGTGTCCCTGACG |
| | GGGGGCCTGAGCTACAAAGAGGACACGAAGGAGCTGGTGGTGGCCAAGGCTG |
| | GAGTCTACTATGTCTTCTTTCAACTAGAGCTGCGGCGCGTGGTGGCCGGCGAGG |
| | GCTCAGGCTCCGTTTCACTTGCGCTGCACCTGCAGCCACTGCGCTCTGCTGCTG |
| | GGGCCGCCGCCCTGGCTTTGACCGTGGACCTGCCACCCGCCTCCTCCGAGGCTC |
| | GGAACTCGGCCTTCGGTTTCCAGGGCCGCTTGCTGCACCTGAGTGCCGGCCAGC |
| | GCCTGGGCGTCCATCTTCACACTGAGGCCAGGGCACGCCATGCCTGGCAGCTTA |
| | CCCAGGGCGCCACAGTCTTGGGACTCTTCCGGGTGACCCCCGAAATCCCAGCCG |
| | GACTCCCTTCACCGAGGTCGGAATAA (SEQ ID NO: 206) |
| | >Artificial sequence, M/P-4F2-sc4-1BBL, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRGGGGSGGGGSGGGGSREGPELSPDDPAGLLDLRQGMFAQLVAQNVLLI |
| | DGPLSWYSDPGLAGVSLTGGLSYKEDTKELVVAKAGVYYVFFQLELRRVVAGEGS |
| | GSVSLALHLQPLRSAAGAAALALTVDLPPASSEARNSAFGFQGRLLHLSAGQRLGV |
| | HLHTEARARHAWQLTQGATVLGLFRVTPEIPAGLPSPRSEGGGGSGGGGSGGGGS |
| | GGGGSREGPELSPDDPAGLLDLRQGMFAQLVAQNVLLIDGPLSWYSDPGLAGVSL |
| | TGGLSYKEDTKELVVAKAGVYYVFFQLELRRVVAGEGSGSVSLALHLQPLRSAAG |
| | AAALALTVDLPPASSEARNSAFGFQGRLLHLSAGQRLGVHLHTEARARHAWQLTQ |
| | GATVLGLFRVTPEIPAGLPSPRSEGGGGSGGGGSGGGGSGGGGSREGPELSPDDPA |
| | GLLDLRQGMFAQLVAQNVLLIDGPLSWYSDPGLAGVSLTGGLSYKEDTKELVVAK |
| | AGVYYVFFQLELRRVVAGEGSGSVSLALHLQPLRSAAGAAALALTVDLPPASSEAR |
| | NSAFGFQGRLLHLSAGQRLGVHLHTEARARHAWQLTQGATVLGLFRVTPEIPAGL |
| | PSPRSE |
| | (SEQ ID NO: 207) |
| Artificial | >Artificial sequence, sc4-1BBL-Fc-CD9tm2, mRNA, |
| Sequence | nucleic acid sequence |
| sc4-1BBL-Fc- | atgtggtggcgactctggtggctgttgcttctgctcttgcttctgtggcccatgg |
| CD9tm2 | tctgggccCGCGAGGGTCCCGAGC |
| | TTTCGCCCGACGATCCCGCCGGCCTCTTGGACCTGCGGCAGGGCATGTTTGCGC |
| | AGCTGGTGGCCCAAAATGTTCTGCTGATCGATGGGCCCCTGAGCTGGTACAGTG |
| | ACCCAGGCCTGGCAGGCGTGTCCCTGACGGGGGGCCTGAGCTACAAAGAGGAC |
| | ACGAAGGAGCTGGTGGTGGCCAAGGCTGGAGTCTACTATGTCTTCTTTCAACTA |
| | GAGCTGCGGCGCGTGGTGGCCGGCGAGGGCTCAGGCTCCGTTTCACTTGCGCTG |
| | CACCTGCAGCCACTGCGCTCTGCTGCTGGGGCCGCCGCCCTGGCTTTGACCGTG |
| | GACCTGCCACCCGCCTCCTCCGAGGCTCGGAACTCGGCCTTCGGTTTCCAGGGC |
| | CGCTTGCTGCACCTGAGTGCCGGCCAGCGCCTGGGCGTCCATCTTCACACTGAG |
| | GCCAGGGCACGCCATGCCTGGCAGCTTACCCAGGGCGCCACAGTCTTGGGACTC |
| | TTCCGGGTGACCCCCGAAATCCCAGCCGGACTCCCTTCACCGAGGTCGGAAGGT |
| | GGCGGTGGCTCCGGAGGTGGCGGTTCCGGTGGCGGTGGCTCCGGTGGCGGTGG |
| | CTCCCGCGAGGGTCCCGAGCTTTCGCCCGACGATCCCGCCGGCCTCTTGGACCT |
| | GCGGCAGGGCATGTTTGCGCAGCTGGTGGCCCAAAATGTTCTGCTGATCGATGG |
| | GCCCCTGAGCTGGTACAGTGACCCAGGCCTGGCAGGCGTGTCCCTGACGGGGG |
| | GCCTGAGCTACAAAGAGGACACGAAGGAGCTGGTGGTGGCCAAGGCTGGAGTC |
| | TACTATGTCTTCTTTCAACTAGAGCTGCGGCGCGTGGTGGCCGGCGAGGGCTCA |
| | GGCTCCGTTTCACTTGCGCTGCACCTGCAGCCACTGCGCTCTGCTGCTGGGGCC |
| | GCCGCCCTGGCTTTGACCGTGGACCTGCCACCCGCCTCCTCCGAGGCTCGGAAC |
| | TCGGCCTTCGGTTTCCAGGGCCGCTTGCTGCACCTGAGTGCCGGCCAGCGCCTG |
| | GGCGTCCATCTTCACACTGAGGCCAGGGCACGCCATGCCTGGCAGCTTACCCAG |
| | GGCGCCACAGTCTTGGGACTCTTCCGGGTGACCCCCGAAATCCCAGCCGGACTC |
| | CCTTCACCGAGGTCGGAAGGTGGCGGTGGCTCCGGTGGCGGTGGCTCCGGTGG |
| | CGGTGGCTCCGGAGGTGGCGGCTCCCGCGAGGGTCCCGAGCTTTCGCCCGACG |
| | ATCCCGCCGGCCTCTTGGACCTGCGGCAGGGCATGTTTGCGCAGCTGGTGGCCC |
| | AAAATGTTCTGCTGATCGATGGGCCCCTGAGCTGGTACAGTGACCCAGGCCTGG |
| | CAGGCGTGTCCCTGACGGGGGGCCTGAGCTACAAAGAGGACACGAAGGAGCTG |
| | GTGGTGGCCAAGGCTGGAGTCTACTATGTCTTCTTTCAACTAGAGCTGCGGCGC |
| | GTGGTGGCCGGCGAGGGCTCAGGCTCCGTTTCACTTGCGCTGCACCTGCAGCCA |
| | CTGCGCTCTGCTGCTGGGGCCGCCGCCCTGGCTTTGACCGTGGACCTGCCACCC |
| | GCCTCCTCCGAGGCTCGGAACTCGGCCTTCGGTTTCCAGGGCCGCTTGCTGCAC |
| | CTGAGTGCCGGCCAGCGCCTGGGCGTCCATCTTCACACTGAGGCCAGGGCACGC |
| | CATGCCTGGCAGCTTACCCAGGGCGCCACAGTCTTGGGACTCTTCCGGGTGACC |
| | CCCGAAATCCCAGCCGGACTCCCTTCACCGAGGTCGGAAGGTGGCGGTGGCTCC |
| | GGTGGCGGTGGCTCCGGTGGCGGTGGCTCCGGTGGCGGTGGATCCGACAAAAC |
| | TCACACATGCCCACCGTGCCCAGCACCTGAAGCAGCCGGGGGACCGTCAGTCTT |
| | CCTCTTCCCCCCAAAACCCAAGGACACCCTCATGATCTCCCGGACCCCTGAGGT |
| | CACATGCGTGGTGGTGGACGTGAGCCACGAAGACCCTGAGGTCAAGTTCAACT |
| | GGTACGTGGACGGCGTGGAGGTGCATAATGCCAAGACAAAGCCGCGGGAGGA |
| | GCAGTACAACAGCACGTACCGTGTGGTCAGCGTCCTCACCGTCCTGCACCAGGA |
| | CTGGCTGAATGGCAAGGAGTACAAGTGCAAGGTCTCCAACAAAGCCCTCGGCG |
| | CCCCCATCGAGAAAACCATCTCCAAAGCCAAAGGGCAGCCCCGAGAACCACAG |
| | GTGTACACCCTGCCCCCATCCCGGGAGGAGATGACCAAGAACCAGGTCAGCCT |
| | GACCTGCCTGGTCAAAGGCTTCTATCCCAGCGACATCGCCGTGGAGTGGGAGA |
| | GCAATGGGCAGCCGGAGAACAACTACAAGACCACGCCTCCCGTGCTGGACTCC |
| | GACGGCTCCTTCTTCCTCTACAGCAAGCTCACCGTGGACAAGAGCAGGTGGCAG |
| | CAGGGGAACGTCTTCTCATGCTCCGTGATGCACGAGGCTCTGCACAACCACTAC |
| | ACGCAGAAGAGCCTCTCCCTGTCTCCGGGTAAAATCGATTTCTACACAGGAGTC |
| | TATATTCTGATCGGAGCCGGCGCCCTCATGATGCTGGTGGGCTTCCTGGGCTGC |
| | TGCGGGGCTGTGCAGGAGTCCCAGTGCGTAATTATGTAA (SEQ ID NO: 208) |
| | >Artificial sequence, sc4-1BBL-Fc-CD9tm2, amino acid |
| | sequence |
| | MWWRLWWLLLLLLLLWPMVWAREGPELSPDDPAGLLDLRQGMFAQLVAQNVLL |
| | IDGPLSWYSDPGLAGVSLTGGLSYKEDTKELVVAKAGVYYVFFQLELRRVVAGEG |
| | SGSVSLALHLQPLRSAAGAAALALTVDLPPASSEARNSAFGFQGRLLHLSAGQRLG |
| | VHLHTEARARHAWQLTQGATVLGLFRVTPEIPAGLPSPRSEGGGGSGGGGSGGGG |
| | SGGGGSREGPELSPDDPAGLLDLRQGMFAQLVAQNVLLIDGPLSWYSDPGLAGVS |
| | LTGGLSYKEDTKELVVAKAGVYYVFFQLELRRVVAGEGSGSVSLALHLQPLRSAA |
| | GAAALALTVDLPPASSEARNSAFGFQGRLLHLSAGQRLGVHLHTEARARHAWQLT |
| | QGATVLGLFRVTPEIPAGLPSPRSEGGGGSGGGGSGGGGSGGGGSREGPELSPDDP |
| | AGLLDLRQGMFAQLVAQNVLLIDGPLSWYSDPGLAGVSLTGGLSYKEDTKELVVA |
| | KAGVYYVFFQLELRRVVAGEGSGSVSLALHLQPLRSAAGAAALALTVDLPPASSE |
| | ARNSAFGFQGRLLHLSAGQRLGVHLHTEARARHAWQLTQGATVLGLFRVTPEIPA |
| | GLPSPRSEGGGGSGGGGSGGGGSGGGGSDKTHTCPPCPAPEAAGGPSVFLFPPKPK |
| | DTLMISRTPEVTCVVVDVSHEDPEVKFNWYVDGVEVHNAKTKPREEQYNSTYRV |
| | VSVLTVLHQDWLNGKEYKCKVSNKALGAPIEKTISKAKGQPREPQVYTLPPSREEM |
| | TKNQVSLTCLVKGFYPSDIAVEWESNGQPENNYKTTPPVLDSDGSFFLYSKLTVDK |
| | SRWQQGNVFSCSVMHEALHNHYTQKSLSLSPGKIDFYTGVYILIGAGALMMLVGF |
| | LGCCGAVQESQCVIM (SEQ ID NO: 209) |
| Artificial | >Artificial sequence, M/P-CD298-Fc-sc4-1BBL, mRNA, |
| sequence, | nucleic acid sequence |
| M/P-CD298- | ATGGGTTGCTGTTTCTCCAAGACCGGCTCGAGCGGCACGAAGAACGAGAAGAA |
| Fc-sc4-1BBL | GTCCCTCAACCAGAGCCTGGCCGAGTGGAAGCTCTTCATCTACAACCCGACCAC |
| | CGGAGAATTCCTGGGGCGCACCGCCAAGAGCTGGGGTTTGATCTTGCTCTTCTA |
| | CCTAGTTTTTTATGGGTTCCTGGCTGCACTCTTCTCATTCACGCCGTGCCCAGCA |
| | CCTGAAGCAGCCGGGGGACCGTCAGTCTTCCTCTTCCCCCCAAAACCCAAGGAC |
| | ACCCTCATGATCTCCCGGACCCCTGAGGTCACATGCGTGGTGGTGGACGTGAGC |
| | CACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTGTGG |
| | TCAGCGTCCTCACCGTCCTGCACCAGGACTGGCTGAATGGCAAGGAGTACAAGT |
| | GCAAGGTCTCCAACAAAGCCCTCGGCGCCCCCATCGAGAAAACCATCTCCAAA |
| | GCCAAAGGGCAGCCCCGAGAACCACAGGTGTACACCCTGCCCCCATCCCGGGA |
| | TGAGCTGACCAAGAACCAGGTCAGCCTGACCTGCCTGGTCAAAGGCTTCTATCC |
| | CAGCGACATCGCCGTGGAGTGGGAGAGCAATGGGCAGCCGGAGAACAACTACA |
| | AGACCACGCCTCCCGTGCTGGACTCCGACGGCTCCTTCTTCCTCTACAGCAAGC |
| | TCACCGTGGACAAGAGCAGGTGGCAGCAGGGGAACGTCTTCTCATGCTCCGTG |
| | ATGCATGAGGCTCTGCACAACCACTACACGCAGAAGAGCCTCTCCCTGTCTCCG |
| | GGTAAAGGTGGCGGTGGCTCCGGTGGCGGTGGCTCCGGTGGCGGTGGATCCCG |
| | CGAGGGTCCCGAGCTTTCGCCCGACGATCCCGCCGGCCTCTTGGACCTGCGGCA |
| | GGGCATGTTTGCGCAGCTGGTGGCCCAAAATGTTCTGCTGATCGATGGGCCCCT |
| | GAGCTGGTACAGTGACCCAGGCCTGGCAGGCGTGTCCCTGACGGGGGGCCTGA |
| | GCTACAAAGAGGACACGAAGGAGCTGGTGGTGGCCAAGGCTGGAGTCTACTAT |
| | GTCTTCTTTCAACTAGAGCTGCGGCGCGTGGTGGCCGGCGAGGGCTCAGGCTCC |
| | GTTTCACTTGCGCTGCACCTGCAGCCACTGCGCTCTGCTGCTGGGGCCGCCGCC |
| | CTGGCTTTGACCGTGGACCTGCCACCCGCCTCCTCCGAGGCTCGGAACTCGGCC |
| | TTCGGTTTCCAGGGCCGCTTGCTGCACCTGAGTGCCGGCCAGCGCCTGGGCGTC |
| | CATCTTCACACTGAGGCCAGGGCACGCCATGCCTGGCAGCTTACCCAGGGCGCC |
| | ACAGTCTTGGGACTCTTCCGGGTGACCCCCGAAATCCCAGCCGGACTCCCTTCA |
| | CCGAGGTCGGAAGGTGGCGGTGGCTCCGGAGGTGGCGGTTCCGGTGGCGGTGG |
| | CTCCGGTGGCGGTGGCTCCCGCGAGGGTCCCGAGCTTTCGCCCGACGATCCCGC |
| | CGGCCTCTTGGACCTGCGGCAGGGCATGTTTGCGCAGCTGGTGGCCCAAAATGT |
| | TCTGCTGATCGATGGGCCCCTGAGCTGGTACAGTGACCCAGGCCTGGCAGGCGT |
| | GTCCCTGACGGGGGGCCTGAGCTACAAAGAGGACACGAAGGAGCTGGTGGTGG |
| | CCAAGGCTGGAGTCTACTATGTCTTCTTTCAACTAGAGCTGCGGCGCGTGGTGG |
| | CCGGCGAGGGCTCAGGCTCCGTTTCACTTGCGCTGCACCTGCAGCCACTGCGCT |
| | CTGCTGCTGGGGCCGCCGCCCTGGCTTTGACCGTGGACCTGCCACCCGCCTCCT |
| | CCGAGGCTCGGAACTCGGCCTTCGGTTTCCAGGGCCGCTTGCTGCACCTGAGTG |
| | CCGGCCAGCGCCTGGGCGTCCATCTTCACACTGAGGCCAGGGCACGCCATGCCT |
| | GGCAGCTTACCCAGGGCGCCACAGTCTTGGGACTCTTCCGGGTGACCCCCGAAA |
| | TCCCAGCCGGACTCCCTTCACCGAGGTCGGAAGGTGGCGGTGGCTCCGGTGGCG |
| | GTGGCTCCGGTGGCGGTGGCTCCGGAGGTGGCGGCTCCCGCGAGGGTCCCGAG |
| | CTTTCGCCCGACGATCCCGCCGGCCTCTTGGACCTGCGGCAGGGCATGTTTGCG |
| | CAGCTGGTGGCCCAAAATGTTCTGCTGATCGATGGGCCCCTGAGCTGGTACAGT |
| | GACCCAGGCCTGGCAGGCGTGTCCCTGACGGGGGGCCTGAGCTACAAAGAGGA |
| | CACGAAGGAGCTGGTGGTGGCCAAGGCTGGAGTCTACTATGTCTTCTTTCAACT |
| | AGAGCTGCGGCGCGTGGTGGCCGGCGAGGGCTCAGGCTCCGTTTCACTTGCGCT |
| | GCACCTGCAGCCACTGCGCTCTGCTGCTGGGGCCGCCGCCCTGGCTTTGACCGT |
| | GGACCTGCCACCCGCCTCCTCCGAGGCTCGGAACTCGGCCTTCGGTTTCCAGGG |
| | CCGCTTGCTGCACCTGAGTGCCGGCCAGCGCCTGGGCGTCCATCTTCACACTGA |
| | GGCCAGGGCACGCCATGCCTGGCAGCTTACCCAGGGCGCCACAGTCTTGGGAC |
| | TCTTCCGGGTGACCCCCGAAATCCCAGCCGGACTCCCTTCACCGAGGTCGGAAT |
| | AA |
| | (SEQ ID NO: 210) |
| | >Artificial sequence, M/P-CD298-Fc-sc4-1BBL, amino acid |
| | sequence |
| | MGCCFSKTGSSGTKNEKKSLNQSLAEWKLFIYNPTTGEFLGRTAKSWGLILLFYLV |
| | FYGFLAALFSFTPCPAPEAAGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALGAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGKGGGGSGGGGSGGGGSREGPELSPDDPAGLLDLRQGMFAQLVAQNV |
| | LLIDGPLSWYSDPGLAGVSLTGGLSYKEDTKELVVAKAGVYYVFFQLELRRVVAG |
| | EGSGSVSLALHLQPLRSAAGAAALALTVDLPPASSEARNSAFGFQGRLLHLSAGQR |
| | LGVHLHTEARARHAWQLTQGATVLGLFRVTPEIPAGLPSPRSEGGGGSGGGGSGG |
| | GGSGGGGSREGPELSPDDPAGLLDLRQGMFAQLVAQNVLLIDGPLSWYSDPGLAG |
| | VSLTGGLSYKEDTKELVVAKAGVYYVFFQLELRRVVAGEGSGSVSLALHLQPLRS |
| | AAGAAALALTVDLPPASSEARNSAFGFQGRLLHLSAGQRLGVHLHTEARARHAW |
| | QLTQGATVLGLFRVTPEIPAGLPSPRSEGGGGSGGGGSGGGGSGGGGSREGPELSPD |
| | DPAGLLDLRQGMFAQLVAQNVLLIDGPLSWYSDPGLAGVSLTGGLSYKEDTKELV |
| | VAKAGVYYVFFQLELRRVVAGEGSGSVSLALHLQPLRSAAGAAALALTVDLPPAS |
| | SEARNSAFGFQGRLLHLSAGQRLGVHLHTEARARHAWQLTQGATVLGLFRVTPEIP |
| | AGLPSPRSE (SEQ ID NO: 211) |
| Artificial | >Artificial sequence, M/P-CD298-Fc-scGITRL, mRNA, |
| sequence, | nucleic sequence |
| M/P-CD298- | ATGGGTTGCTGTTTCTCCAAGACCGGCTCGAGCGGCACGAAGAACGAGAAGAA |
| Fc-scGITRL | GTCCCTCAACCAGAGCCTGGCCGAGTGGAAGCTCTTCATCTACAACCCGACCAC |
| | CGGAGAATTCCTGGGGCGCACCGCCAAGAGCTGGGGTTTGATCTTGCTCTTCTA |
| | CCTAGTTTTTTATGGGTTCCTGGCTGCACTCTTCTCATTCACGCCGTGCCCAGCA |
| | CCTGAAGCAGCCGGGGGACCGTCAGTCTTCCTCTTCCCCCCAAAACCCAAGGAC |
| | ACCCTCATGATCTCCCGGACCCCTGAGGTCACATGCGTGGTGGTGGACGTGAGC |
| | CACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTGTGG |
| | TCAGCGTCCTCACCGTCCTGCACCAGGACTGGCTGAATGGCAAGGAGTACAAGT |
| | GCAAGGTCTCCAACAAAGCCCTCGGCGCCCCCATCGAGAAAACCATCTCCAAA |
| | GCCAAAGGGCAGCCCCGAGAACCACAGGTGTACACCCTGCCCCCATCCCGGGA |
| | TGAGCTGACCAAGAACCAGGTCAGCCTGACCTGCCTGGTCAAAGGCTTCTATCC |
| | CAGCGACATCGCCGTGGAGTGGGAGAGCAATGGGCAGCCGGAGAACAACTACA |
| | AGACCACGCCTCCCGTGCTGGACTCCGACGGCTCCTTCTTCCTCTACAGCAAGC |
| | TCACCGTGGACAAGAGCAGGTGGCAGCAGGGGAACGTCTTCTCATGCTCCGTG |
| | ATGCATGAGGCTCTGCACAACCACTACACGCAGAAGAGCCTCTCCCTGTCTCCG |
| | GGTAAAGGTGGCGGTGGCTCCGGTGGCGGTGGCTCCGGTGGCGGTGGATCCGA |
| | GCCCTGTATGGCTAAGTTTGGACCATTACCCTCAAAATGGCAAATGGCATCTTC |
| | TGAACCTCCTTGCGTGAATAAGGTGTCTGACTGGAAGCTGGAGATACTTCAGAA |
| | TGGCTTATATTTAATTTATGGCCAAGTGGCTCCCAATGCAAACTACAATGATGT |
| | AGCTCCTTTTGAGGTGCGGCTGTATAAAAACAAAGACATGATACAAACTCTAAC |
| | AAACAAATCTAAAATCCAAAATGTAGGAGGGACTTATGAATTGCATGTTGGGG |
| | ACACCATAGACTTGATATTCAACTCTGAGCATCAGGTTCTAAAAAATAATACAT |
| | ACTGGGGTATCATTTTACTAGCAAATCCCCAATTCATCTCCGGTGGCGGTGGCT |
| | CCGGAGGTGGCGGTTCCGAGCCCTGTATGGCTAAGTTTGGACCATTACCCTCAA |
| | AATGGCAAATGGCATCTTCTGAACCTCCTTGCGTGAATAAGGTGTCTGACTGGA |
| | AGCTGGAGATACTTCAGAATGGCTTATATTTAATTTATGGCCAAGTGGCTCCCA |
| | ATGCAAACTACAATGATGTAGCTCCTTTTGAGGTGCGGCTGTATAAAAACAAAG |
| | ACATGATACAAACTCTAACAAACAAATCTAAAATCCAAAATGTAGGAGGGACT |
| | TATGAATTGCATGTTGGGGACACCATAGACTTGATATTCAACTCTGAGCATCAG |
| | GTTCTAAAAAATAATACATACTGGGGTATCATTTTACTAGCAAATCCCCAATTC |
| | ATCTCCGGTGGCGGTGGCTCCGGTGGCGGTGGCTCCGAGCCCTGTATGGCTAAG |
| | TTTGGACCATTACCCTCAAAATGGCAAATGGCATCTTCTGAACCTCCTTGCGTG |
| | AATAAGGTGTCTGACTGGAAGCTGGAGATACTTCAGAATGGCTTATATTTAATT |
| | TATGGCCAAGTGGCTCCCAATGCAAACTACAATGATGTAGCTCCTTTTGAGGTG |
| | CGGCTGTATAAAAACAAAGACATGATACAAACTCTAACAAACAAATCTAAAAT |
| | CCAAAATGTAGGAGGGACTTATGAATTGCATGTTGGGGACACCATAGACTTGAT |
| | ATTCAACTCTGAGCATCAGGTTCTAAAAAATAATACATACTGGGGTATCATTTT |
| | ACTAGCAAATCCCCAATTCATCTCCTAG (SEQ ID NO: 212) |
| | >Artificial sequence, M/P-CD298-Fc-scGITRL, amino acid |
| | sequence |
| | MGCCFSKTGSSGTKNEKKSLNQSLAEWKLFIYNPTTGEFLGRTAKSWGLILLFYLV |
| | FYGFLAALFSFTPCPAPEAAGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALGAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGKGGGGSGGGGSGGGGSEPCMAKFGPLPSKWQMASSEPPCVNKVSD |
| | WKLEILQNGLYLIYGQVAPNANYNDVAPFEVRLYKNKDMIQTLINKSKIQNVGGT |
| | YELHVGDTIDLIFNSEHQVLKNNTYWGIILLANPQFISGGGGSGGGGSEPCMAKFGP |
| | LPSKWQMASSEPPCVNKVSDWKLEILQNGLYLIYGQVAPNANYNDVAPFEVRLYK |
| | NKDMIQTLTNKSKIQNVGGTYELHVGDTIDLIFNSEHQVLKNNTYWGIILLANPQFI |
| | SGGGGSGGGGSEPCMAKFGPLPSKWQMASSEPPCVNKVSDWKLEILQNGLYLIYG |
| | QVAPNANYNDVAPFEVRLYKNKDMIQTLTNKSKIQNVGGTYELHVGDTIDLIFNSE |
| | HQVLKNNTYWGIILLANPQFIS (SEQ ID NO: 213) |
| Artificial | >Artificial sequence, M/P-CD298-Fc-scOX40L, mRNA, |
| sequence, | nucleic acid sequence |
| M/P-CD298- | ATGGGTTGCTGTTTCTCCAAGACCGGCTCGAGCGGCACGAAGAACGAGAAGAA |
| Fc-scOX40L | GTCCCTCAACCAGAGCCTGGCCGAGTGGAAGCTCTTCATCTACAACCCGACCAC |
| | CGGAGAATTCCTGGGGCGCACCGCCAAGAGCTGGGGTTTGATCTTGCTCTTCTA |
| | CCTAGTTTTTTATGGGTTCCTGGCTGCACTCTTCTCATTCACGCCGTGCCCAGCA |
| | CCTGAAGCAGCCGGGGGACCGTCAGTCTTCCTCTTCCCCCCAAAACCCAAGGAC |
| | ACCCTCATGATCTCCCGGACCCCTGAGGTCACATGCGTGGTGGTGGACGTGAGC |
| | CACGAAGACCCTGAGGTCAAGTTCAACTGGTACGTGGACGGCGTGGAGGTGCA |
| | TAATGCCAAGACAAAGCCGCGGGAGGAGCAGTACAACAGCACGTACCGTGTGG |
| | TCAGCGTCCTCACCGTCCTGCACCAGGACTGGCTGAATGGCAAGGAGTACAAGT |
| | GCAAGGTCTCCAACAAAGCCCTCGGCGCCCCCATCGAGAAAACCATCTCCAAA |
| | GCCAAAGGGCAGCCCCGAGAACCACAGGTGTACACCCTGCCCCCATCCCGGGA |
| | TGAGCTGACCAAGAACCAGGTCAGCCTGACCTGCCTGGTCAAAGGCTTCTATCC |
| | CAGCGACATCGCCGTGGAGTGGGAGAGCAATGGGCAGCCGGAGAACAACTACA |
| | AGACCACGCCTCCCGTGCTGGACTCCGACGGCTCCTTCTTCCTCTACAGCAAGC |
| | TCACCGTGGACAAGAGCAGGTGGCAGCAGGGGAACGTCTTCTCATGCTCCGTG |
| | ATGCATGAGGCTCTGCACAACCACTACACGCAGAAGAGCCTCTCCCTGTCTCCG |
| | GGTAAAGGTGGCGGTGGCTCCGGTGGCGGTGGCTCCGGTGGCGGTGGATCCCA |
| | TCGGTATCCTCGAATTCAAAGTATCAAAGTACAATTTACCGAATATAAGAAGGA |
| | GAAAGGTTTCATCCTCACTTCCCAAAAGGAGGATGAAATCATGAAGGTGCAGA |
| | ACAACTCAGTCATCATCAACTGTGATGGGTTTTATCTCATCTCCCTGAAGGGCT |
| | ACTTCTCCCAGGAAGTCAACATTAGCCTTCATTACCAGAAGGATGAGGAGCCCC |
| | TCTTCCAACTGAAGAAGGTCAGGTCTGTCAACTCCTTGATGGTGGCCTCTCTGA |
| | CTTACAAAGACAAAGTCTACTTGAATGTGACCACTGACAATACCTCCCTGGATG |
| | ACTTCCATGTGAATGGCGGAGAACTGATTCTTATCCATCAAAATCCTGGTGAAT |
| | TCTGTGTCCTTGGTGGCGGTGGCTCCGGAGGTGGCGGTTCCCATCGGTATCCTC |
| | GAATTCAAAGTATCAAAGTACAATTTACCGAATATAAGAAGGAGAAAGGTTTC |
| | ATCCTCACTTCCCAAAAGGAGGATGAAATCATGAAGGTGCAGAACAACTCAGT |
| | CATCATCAACTGTGATGGGTTTTATCTCATCTCCCTGAAGGGCTACTTCTCCCAG |
| | GAAGTCAACATTAGCCTTCATTACCAGAAGGATGAGGAGCCCCTCTTCCAACTG |
| | AAGAAGGTCAGGTCTGTCAACTCCTTGATGGTGGCCTCTCTGACTTACAAAGAC |
| | AAAGTCTACTTGAATGTGACCACTGACAATACCTCCCTGGATGACTTCCATGTG |
| | AATGGCGGAGAACTGATTCTTATCCATCAAAATCCTGGTGAATTCTGTGTCCTT |
| | GGTGGCGGTGGCTCCGGTGGCGGTGGCTCCCATCGGTATCCTCGAATTCAAAGT |
| | ATCAAAGTACAATTTACCGAATATAAGAAGGAGAAAGGTTTCATCCTCACTTCC |
| | CAAAAGGAGGATGAAATCATGAAGGTGCAGAACAACTCAGTCATCATCAACTG |
| | TGATGGGTTTTATCTCATCTCCCTGAAGGGCTACTTCTCCCAGGAAGTCAACATT |
| | AGCCTTCATTACCAGAAGGATGAGGAGCCCCTCTTCCAACTGAAGAAGGTCAG |
| | GTCTGTCAACTCCTTGATGGTGGCCTCTCTGACTTACAAAGACAAAGTCTACTT |
| | GAATGTGACCACTGACAATACCTCCCTGGATGACTTCCATGTGAATGGCGGAGA |
| | ACTGATTCTTATCCATCAAAATCCTGGTGAATTCTGTGTCCTTTGA |
| | (SEQ ID NO: 214) |
| | >Artificial sequence, M/P-CD298-Fc-scOX40L, amino acid |
| | sequence |
| | MGCCFSKTGSSGTKNEKKSLNQSLAEWKLFIYNPTTGEFLGRTAKSWGLILLFYLV |
| | FYGFLAALFSFTPCPAPEAAGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSN |
| | KALGAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYT |
| | QKSLSLSPGKGGGGSGGGGSGGGGSHRYPRIQSIKVQFTEYKKEKGFILTSQKEDEI |
| | MKVQNNSVIINCDGFYLISLKGYFSQEVNISLHYQKDEEPLFQLKKVRSVNSLMVA |
| | SLTYKDKVYLNVTTDNTSLDDFHVNGGELILIHQNPGEFCVLGGGGSGGGGSHRYP |
| | RIQSIKVQFTEYKKEKGFILTSQKEDEIMKVQNNSVIINCDGFYLISLKGYFSQEVNIS |
| | LHYQKDEEPLFQLKKVRSVNSLMVASLTYKDKVYLNVTTDNTSLDDFHVNGGELI |
| | LIHQNPGEFCVLGGGGSGGGGSHRYPRIQSIKVQFTEYKKEKGFILTSQKEDEIMKV |
| | QNNSVIINCDGFYLISLKGYFSQEVNISLHYQKDEEPLFQLKKVRSVNSLMVASLTY |
| | KDKVYLNVTTDNTSLDDFHVNGGELILIHQNPGEFCVL (SEQ ID NO: 215) |
| Artificial | >Artificial sequence, M/P-4F2-Fc-sc4-1BBLv2, mRNA, |
| Sequence | nucleic acid sequence |
| M/P-4F2-Fc- | ATGGGTTGCTGTTTCTCCAAGACCGGCTCGAGCGGCAGCCAGGACACCGAGGT |
| sc41BBLv2 | GGATATGAAGGAGGTGGAGCTGAATGAGTTAGAGCCCGAGAAGCAGCCGATGA |
| | ACGCGGCGTCTGGGGCGGCCATGTCCCTGGCGGGAGCCGAGAAGAATGGTCTG |
| | GTGAAGATCAAGGTGGCGGAAGACGAGGCGGAGGCGGCAGCCGCGGCTAAGT |
| | TCACGGGCCTGTCCAAGGAGGAGCTGCTGAAGGTGGCAGGCAGCCCCGGCTGG |
| | GTACGCACCCGCTGGGCACTGCTGCTGCTCTTCTGGCTCGGCTGGCTCGGCATG |
| | CTTGCTGGTGCCGTGGTCATAATCGTGCGACCGTGCCCAGCACCTGAAGCAGCC |
| | GGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCC |
| | TGAGGTCAAGTTCAACTGGTACGTGGACGGCGTGGAGGTGCATAATGCCAAGA |
| | CAAAGCCGCGGGAGGAGCAGTACAACAGCACGTACCGTGTGGTCAGCGTCCTC |
| | ACCGTCCTGCACCAGGACTGGCTGAATGGCAAGGAGTACAAGTGCAAGGTCTC |
| | CAACAAAGCCCTCGGCGCCCCCATCGAGAAAACCATCTCCAAAGCCAAAGGGC |
| | AGCCCCGAGAACCACAGGTGTACACCCTGCCCCCATCCCGGGATGAGCTGACC |
| | AAGAACCAGGTCAGCCTGACCTGCCTGGTCAAAGGCTTCTATCCCAGCGACATC |
| | GCCGTGGAGTGGGAGAGCAATGGGCAGCCGGAGAACAACTACAAGACCACGC |
| | CTCCCGTGCTGGACTCCGACGGCTCCTTCTTCCTCTACAGCAAGCTCACCGTGG |
| | ACAAGAGCAGGTGGCAGCAGGGGAACGTCTTCTCATGCTCCGTGATGCATGAG |
| | GCTCTGCACAACCACTACACGCAGAAGAGCCTCTCCCTGTCTCCGGGTAAAGGT |
| | GGCGGTGGCTCCGGTGGCGGTGGCTCCGGTGGCGGTGGATCCTTGGACCTGCGG |
| | CAGGGCATGTTTGCGCAGCTGGTGGCCCAAAATGTTCTGCTGATCGATGGGCCC |
| | CTGAGCTGGTACAGTGACCCAGGCCTGGCAGGCGTGTCCCTGACGGGGGGCCT |
| | GAGCTACAAAGAGGACACGAAGGAGCTGGTGGTGGCCAAGGCTGGAGTCTACT |
| | ATGTCTTCTTTCAACTAGAGCTGCGGCGCGTGGTGGCCGGCGAGGGCTCAGGCT |
| | CCGTTTCACTTGCGCTGCACCTGCAGCCACTGCGCTCTGCTGCTGGGGCCGCCG |
| | CCCTGGCTTTGACCGTGGACCTGCCACCCGCCTCCTCCGAGGCTCGGAACTCGG |
| | CCTTCGGTTTCCAGGGCCGCTTGCTGCACCTGAGTGCCGGCCAGCGCCTGGGCG |
| | TCCATCTTCACACTGAGGCCAGGGCACGCCATGCCTGGCAGCTTACCCAGGGCG |
| | CCACAGTCTTGGGACTCTTCCGGGTGGGTGGCGGTGGCTCCGGTGGCGGTGGCT |
| | CCTTGGACCTGCGGCAGGGCATGTTTGCGCAGCTGGTGGCCCAAAATGTTCTGC |
| | TGATCGATGGGCCCCTGAGCTGGTACAGTGACCCAGGCCTGGCAGGCGTGTCCC |
| | TGACGGGGGGCCTGAGCTACAAAGAGGACACGAAGGAGCTGGTGGTGGCCAA |
| | GGCTGGAGTCTACTATGTCTTCTTTCAACTAGAGCTGCGGCGCGTGGTGGCCGG |
| | CGAGGGCTCAGGCTCCGTTTCACTTGCGCTGCACCTGCAGCCACTGCGCTCTGC |
| | TGCTGGGGCCGCCGCCCTGGCTTTGACCGTGGACCTGCCACCCGCCTCCTCCGA |
| | GGCTCGGAACTCGGCCTTCGGTTTCCAGGGCCGCTTGCTGCACCTGAGTGCCGG |
| | CCAGCGCCTGGGCGTCCATCTTCACACTGAGGCCAGGGCACGCCATGCCTGGCA |
| | GCTTACCCAGGGCGCCACAGTCTTGGGACTCTTCCGGGTGGGTGGCGGTGGCTC |
| | CGGAGGTGGCGGCTCCTTGGACCTGCGGCAGGGCATGTTTGCGCAGCTGGTGGC |
| | CCAAAATGTTCTGCTGATCGATGGGCCCCTGAGCTGGTACAGTGACCCAGGCCT |
| | GGCAGGCGTGTCCCTGACGGGGGGCCTGAGCTACAAAGAGGACACGAAGGAGC |
| | TGGTGGTGGCCAAGGCTGGAGTCTACTATGTCTTCTTTCAACTAGAGCTGCGGC |
| | GCGTGGTGGCCGGCGAGGGCTCAGGCTCCGTTTCACTTGCGCTGCACCTGCAGC |
| | CACTGCGCTCTGCTGCTGGGGCCGCCGCCCTGGCTTTGACCGTGGACCTGCCAC |
| | CCGCCTCCTCCGAGGCTCGGAACTCGGCCTTCGGTTTCCAGGGCCGCTTGCTGC |
| | ACCTGAGTGCCGGCCAGCGCCTGGGCGTCCATCTTCACACTGAGGCCAGGGCAC |
| | GCCATGCCTGGCAGCTTACCCAGGGCGCCACAGTCTTGGGACTCTTCCGGGTGA |
| | CCCCCGAAATCCCAGCCGGACTCCCTTCACCGAGGTCGGAATAA (SEQ ID NO: |
| | 216) |
| | >Artificial sequence, M/P-4F2-Fc-sc4-1BBLv2, amino acid |
| | sequence |
| | MGCCFSKTGSSGSQDTEVDMKEVELNELEPEKQPMNAASGAAMSLAGAEKNGLV |
| | KIKVAEDEAEAAAAAKFTGLSKEELLKVAGSPGWVRTRWALLLLFWLGWLGMLA |
| | GAVVIIVRPCPAPEAAGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALG |
| | APIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGKGGGGSGGGGSGGGGSLDLRQGMFAQLVAQNVLLIDGPLSWYSDPGLAGV |
| | SLTGGLSYKEDTKELVVAKAGVYYVFFQLELRRVVAGEGSGSVSLALHLQPLRSA |
| | AGAAALALTVDLPPASSEARNSAFGFQGRLLHLSAGQRLGVHLHTEARARHAWQL |
| | TQGATVLGLFRVGGGGSGGGGSLDLRQGMFAQLVAQNVLLIDGPLSWYSDPGLA |
| | GVSLTGGLSYKEDTKELVVAKAGVYYVFFQLELRRVVAGEGSGSVSLALHLQPLR |
| | SAAGAAALALTVDLPPASSEARNSAFGFQGRLLHLSAGQRLGVHLHTEARARHAW |
| | QLTQGATVLGLFRVGGGGSGGGGSLDLRQGMFAQLVAQNVLLIDGPLSWYSDPGL |
| | AGVSLTGGLSYKEDTKELVVAKAGVYYVFFQLELRRVVAGEGSGSVSLALHLQPL |
| | RSAAGAAALALTVDLPPASSEARNSAFGFQGRLLHLSAGQRLGVHLHTEARARHA |
| | WQLTQGATVLGLFRVTPEIPAGLPSPRSE (SEQ ID NO: 217) |

In some embodiments of any of the aspects, the fusion polypeptides provided herein comprise two or more POI domains. The specific combinations of POI domains can be used to regulate immune cell responses.

In some embodiments of any of the fusion polypeptide presented herein, there may be a plurality of POI domains or fragments thereof in a single fusion polypeptide sequence. For example, a single fusion polypeptide may have one, two, three, four, five, six, seven, eight, nine, ten, or more POI domains or fragments thereof. In some embodiments, the plurality of POI domains or fragments thereof are the same POI domain or fragment, e.g., repeats of the same POI domain or fragment thereof in a single fusion polypeptide. In other embodiments, the fusion polypeptide comprises various POI domains or POI fragment combinations as provided herein, wherein one fusion polypeptide may comprise two or more different POI domains or fragments thereof.

Non-limiting examples combinations of POIs that can modulate immune receptor signaling pathways are provided in Table 7 (below). The combination of POIs may have an additive or synergistic effect to modulate immune receptor signaling pathways. Exemplary POI combinations within a single fusion polypeptide for modulating immune receptor signaling pathways and immune cell responses are represented in Table 7. It is understood that in any aspect of the fusion polypeptide presented herein the POI may refer to a domain of a POI or a fragment of the POI. It is understood that in any aspect of the fusion polypeptide presented herein the POI combinations referenced in Table 7 may be multi-effector domains.

TABLE 7
Exemplary Fusion Polypeptide POI combination embodiments

| | | | | | | |
|---|---|---|---|---|---|---|
| 41BBL | GITRL | OX40L | CD27L | CD30L | CD40L | LIGHT |
| OX40L | CD27L | CD30L | CD40L | LIGHT | TRAIL | RANKL |
| 41BBL | GITRL | OX40L | CD27L | CD30L | CD40L | LIGHT |
| CD27L | CD30L | CD40L | LIGHT | TRAIL | RANKL | TLIA |
| 41BBL | GITRL | OX40L | CD27L | CD30L | CD40L | LIGHT |
| CD30L | CD40L | LIGHT | TRAIL | RANKL | TLIA | FASL |
| 41BBL | GITRL | OX40L | CD27L | CD30L | CD40L | LIGHT |
| CD40L | LIGHT | TRAIL | RANKL | TL1A | FASL | BAFF |
| 41BBL | GITRL | OX40L | CD27L | CD30L | CD40L | LIGHT |
| LIGHT | TRAIL | RANKL | TLIA | FASL | BAFF | APRIL |
| 41BBL | GITRL | OX40L | CD27L | CD30L | CD40L | LIGHT |
| TRAIL | RANKL | TL1A | FASL | BAFF | APRIL | TWEAK |
| 41BBL | GITRL | OX40L | CD27L | CD30L | CD40L | TRAIL |
| RANKL | TLIA | FASL | BAFF | APRIL | TWEAK | RANKL |
| 41BBL | GITRL | OX40L | CD27L | CD30L | RANKL | TRAIL |
| TL1A | FASL | BAFF | APRIL | TWEAK | TLIA | TL1A |
| 41BBL | GITRL | OX40L | CD27L | TLIA | RANKL | TRAIL |
| FASL | BAFF | APRIL | TWEAK | FASL | FASL | FASL |
| 41BBL | GITRL | OX40L | FASL | TL1A | RANKL | TRAIL |
| BAFF | APRIL | TWEAK | BAFF | BAFF | BAFF | BAFF |
| 41BBL | GITRL | BAFF | FASL | TL1A | RANKL | TRAIL |
| APRIL | TWEAK | APRIL | APRIL | APRIL | APRIL | APRIL |
| 41BBL | APRIL | BAFF | FASL | TLIA | RANKL | TRAIL |
| TWEAK | TWEAK | TWEAK | TWEAK | TWEAK | TWEAK | TWEAK |
| 41BBL | GITRL | OX40L | CD27L | CD30L | CD40L | LIGHT |
| TNF | TNF | TNF | TNF | TNF | TNF | TNF |
| 41BBL | GITRL | OX40L | CD27L | CD30L | CD40L | LIGHT |
| LTA | LTA | LTA | LTA | LTA | LTA | LTA |
| 41BBL | GITRL | OX40L | CD27L | CD30L | CD40L | LIGHT |
| LTB | LTB | LTB | LTB | LTB | LTB | LTB |
| 41BBL | GITRL | OX40L | CD27L | CD30L | CD40L | LIGHT |
| Ectodysplasin | Ectodysplasin | Ectodysplasin | Ectodysplasin | Ectodysplasin | Ectodysplasin | Ectodysplasin |
| A | A | A | A | A | A | A |
| TRAIL | RANKL | TWEAK | APRIL | TLIA | FASL | BAFF |
| TNF | TNF | TNF | TNF | TNF | TNF | TNF |
| TRAIL | RANKL | TWEAK | APRIL | TL1A | FASL | BAFF |
| TNFβ | TNFβ | TNFβ | TNFβ | TNFβ | TNFβ | TNFβ |
| TRAIL | RANKL | TWEAK | APRIL | TLIA | FASL | BAFF |
| LTB | LTB | LTB | LTB | LTB | LTB | LTB |
| TRAIL | RANKL | TWEAK | APRIL | TL1A | FASL | BAFF |
| Ectodysplasin | Ectodysplasin | Ectodysplasin | Ectodysplasin | Ectodysplasin | Ectodysplasin | Ectodysplasin |
| A | A | A | A | A | A | A |
| TNF | TNF | TNF | LTA | LTA | | |
| LTA | LTB | Ectodysplasin | LTB | Ectodysplasin | | |
| | | A | | A | | |

In some embodiments of any of the aspects, the extracellular vesicle provided herein comprise two or more fusion polypeptides. Various combinations of fusion polypeptides displayed on an extracellular vesicle can be used to regulate immune cell responses.

In some embodiments of any of the aspect of extracellular vesicles presented herein, there may be a plurality of fusion polypeptides targeted for display on a single extracellular vesicle. For example, a single extracellular vesicle may have one, two, three, four, five, six, seven, eight, nine, ten, or more fusion polypeptides. In some embodiments, the plurality of fusion polypeptides comprises multiple of the same fusion polypeptide with the same POI. In other embodiments, the plurality of fusion polypeptides displayed on an extracellular vesicle comprise POI combinations.

Non-limiting examples combinations of fusion polypeptides as indicated by their respective POIs that can modulate immune receptor signaling pathways are provided in Table 7 (above). The combination of POIs of a fusion polypeptide may have an additive or synergistic effect to modulate immune receptor signaling pathways. The combination of POIs may have an additive or synergistic effect to agonize (activate/induce) or antagonize (inactivate/suppress) immune receptor signaling pathways. The combination of POIs of a fusion polypeptide may have an additive or synergistic effect to activate, block, agonize, antagonize, or modulate a given target protein, for example agonize a costimulatory or coinhibitory immune checkpoint protein. The combination of POIs of a fusion polypeptide may have an additive or synergistic effect to activate, block, agonize, antagonize, or modulate a given target protein, for example agonize a costimulatory or coinhibitory immune checkpoint protein to induce an immune response.

In some embodiments of any of the extracellular vesicles presented herein, there may be a plurality of fusion polypeptides targeted to a single extracellular vesicle. For example, an extracellular vesicle may have one, two, three, four, five, six, seven, eight, nine, ten, or more fusion polypeptides. In another embodiment, an extracellular vesicle may have between about 1-50, between about 51-100, between about 101-150, between about 151-200, between about 201-250, between about 251-300, between about 301-350, between about 351-400, between about 401-450, between about 451-500, between about 501-550, between about 551-600, between about 601-650, between about 651-700, between about 701-750, between about 751-800, between about 801-950, between about 951-1000, or more fusion polypeptides, each fusion polypeptide of the plurality of fusion polypeptides associated with an extracellular vesicle may be the same or different from one another.

### Methods of Preparing Extracellular Vesicle Compositions

In another aspect, provided herein is a method of preparing an engineered extracellular vesicle provided herein. Generally, the method comprises providing a population of cells expressing a vector construct encoding one or more vesicle targeting domains and one or more signaling domains (POI domain or fragment thereof), and one or more linker. For example, the method comprises providing a population of cells expressing a vector construct encoding one or more fusion polypeptides presented herein. For example, the method comprises providing a population of cells expressing a vector construct encoding one or more fusion polypeptides presented in Table 6. It is understood that the method may also comprises providing a population of cells expressing multiple vector constructs encoding one or more fusion polypeptides in any of the aspects provided herein, for example fusion polypeptides presented in Table 6.

The engineered EVs (e.g., EVs displaying one or more fusion polypeptide embodiments presented therein) provided herein can be isolated and purified form any biological source, e.g., cells. The cells that produce the engineered EVs provided herein can be from any viable non-human source or organism. Usually the organism is an animal, vertebrate, or mammal. In some embodiments, the cell described herein is from a human. The cells described herein can be from any tissue isolated from an organism by methods known in the art. The scientific literature provides guidance for one of ordinary skill in the art to isolate, prepare, and culture cells as necessary for use in the compositions and methods described herein. One of skill in the art can appreciate that the cell source of the EVs may alter the cellular protein expression and the native or endogenous cargo within the EV. It is contemplated herein that this can be leveraged for therapeutic effect depending on the disease or disorder being treated.

In some embodiments, the population of cells has been altered by exposure to environmental conditions (e.g., hypoxia), small molecule addition, presence/absence of exogenous factors (e.g., growth factors, cytokines) at the time, or substantially contemporaneous with, isolating the plurality of engineered extracellular vesicles in a manner altering the regulatory state of the cell. In various embodiments, the cells are human derived GMP-grade cells, for example human embryonic kidney (HEK) 293 cells (e.g., HEK 293F, HEK 293 FT, HEK 293T, HEK 293S, HEK 293FTM, HEK 293SG, HEK 293SGGD, HEK 293H, HEK 293E, HEK 293EBNA1-6E, HEK 293MSR, HEK293A, or the like as known by one of ordinary skill in the art), mesenchymal stem cells (MSCs), umbilical cord MSCs (UC-MSCs), bone marrow MSCs, (BM-MSCs), placental MSCs (P-MSCs), umbilical cord blood MSCs (CD-MSCs), adipose tissue MSCs (A-MSCs), PER.C6, fibrosarcoma HT-1080, HuH7, HeLa, cell lines. In other embodiments, the cells are hematopoietic cells, human embryonic stem cells (hESCs) or hESC derived cells, induced pluripotent stem cells (iPSCs), endothelial progenitor cells (EPCs), or neural stem cells (NSCs), or from primary cells (e.g., human fibroblast cells isolated from juvenile foreskin or from adult skin). In other embodiments, the cells are immune cells, for example lymphocytes (T cells, B cells, and NK cells), neutrophils, and monocytes/macrophages. In other embodiments, the cells are immortalized cell lines including T cell lines such as Jurkat cells, 3T3 cells, A549 cells, HeLa cells, HEK 293 cells, Huh7 cells, OK cells, Ptk2 cells, Vero cells, or the like as known by one of ordinary skill in the art.

In other embodiments the cells are derived from a hamster cell line (e.g., BHK21, CHO cell etc.) or murine cell line (e.g., C127, NS0, Sp2/0, etc.), or canines cell line (e.g., MDCK, IPC-366, TLM-1, CMGD-2, etc.), or monkey cell line (e.g., Vero, etc.), or feline cell line (e.g., CRFK, TiHo-0906, CAT-MT etc.). The cells may be grown in planar culture or suspension culture.

The method comprises providing a population of cells and culturing the cells in serum-free or un-concentrated conditioned medium. This includes, for example, extracellular vesicles secreted into media as conditioned by a population of cells in culture, further including cell lines capable of serial passaging. In certain embodiments, the cells in culture are grown to 10, 20, 30, 40, 50, 60, 70, 80, 90, or 90% or more confluency when engineered extracellular vesicles are isolated.

The methods provided herein further comprise contacting the cells provided herein with a nucleic acid vector encoding the at least one fusion polypeptide provided herein. The vector can be added to the cell culture medium of the cells by methods known in the art as discussed further below.

A vector is a nucleic acid construct designed for delivery to a host cell or for transfer of genetic material between different host cells. As used herein, a vector can be viral or non-viral. The term “vector” encompasses any genetic element that is capable of replication when associated with the proper control elements and that can transfer genetic material to cells. A vector can include, but is not limited to, a cloning vector, an expression vector, a plasmid, phage, transposon, cosmid, artificial chromosome, virus, virion, etc. In some embodiments of any of the aspects, the vector is selected from the group consisting of: a plasmid, a cosmid and a viral vector.

The term “Expression” refers to the cellular processes involved in producing RNA and proteins and as appropriate, secreting proteins, including where applicable, but not limited to, for example, transcription, transcript processing, translation and protein folding, modification and processing. The term “gene products” refers to RNA transcribed from a gene, and polypeptides obtained by translation of mRNA transcribed from a gene.

In some embodiments, a vector is capable of driving expression of one or more sequences in a mammalian cell; i.e., the vector is a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In some embodiments, the recombinant expression vector is capable of directing expression of the exogenous fusion polypeptide nucleic acid sequence preferentially in a particular cell type (e.g., via tissue-specific regulatory elements).

Tissue-specific and inducible regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. hnmunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally regulated promoters are also encompassed, e.g., the murine hox promoters (Kessel and Gruss, 1990. Science 249:374-379) and the a-fetoprotein promoter (Campes and Tilghman, 1989. Genes Dev. 3:537-546).

In some embodiments, the at least one nucleic acid sequence described herein is delivered to the cell described herein via an integrating vector. Integrating vectors have their delivered genetic material (or a copy of it) permanently incorporated into a host cell chromosome. Non-integrating vectors remain episomal which means the nucleic acid contained therein is never integrated into a host cell chromosome. Examples of integrating vectors include retroviral vectors, lentiviral vectors, hybrid adenoviral vectors, and herpes simplex viral vectors.

In some embodiments, the at least one nucleic acid sequence described herein is delivered to the cell described herein via a non-integrative vector. Non-integrative vectors include non-integrative viral vectors. Non-integrative viral vectors eliminate one of the primary risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. Containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free host cells. Other non-integrative viral vectors include adenoviral vectors and the adeno-associated viral (AAV) vectors.

Another non-integrative viral vector is RNA Sendai viral vector, which can produce protein without entering the nucleus of an infected cell. The F-deficient Sendai virus vector remains in the cytoplasm of infected cells for a few passages but is diluted out quickly and completely lost after several passages (e.g., 10 passages). This permits a self-limiting transient expression of a chosen heterologous gene or genes in a target cell. This aspect can be helpful, e.g., for the transient introduction of reprogramming factors, among other uses. As noted above, in some embodiments, the nucleic acid sequence described herein is expressed in the cells from a viral vector.

A “viral vector” includes a nucleic acid vector construct that includes at least one element of viral origin and has the capacity to be packaged into a viral vector particle. The viral vector can contain a nucleic acid encoding a polypeptide described herein in place of non-essential viral genes. The vector and/or particle can be utilized for the purpose of transferring nucleic acids into cells either in vitro or in vivo.

The nucleic acids described herein can be delivered using any transfection reagent or other physical method that facilitates entry of nucleic acids into a cell. Methods of non-viral delivery of nucleic acids include lipofection, nucleofection, microinjection, electroporation, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g., in vitro or ex vivo administration) or target tissues (e.g., in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

An “agent that increases cellular uptake” is a molecule that facilitates transport of a molecule, e.g., nucleic acid, or peptide or polypeptide, or other molecule that does not otherwise efficiently transit the cell membrane across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), or a polyamine (e.g., spermine). Further examples of agents that increase cellular uptake are disclosed, for example, in Winkler (2013). Oligonucleotide conjugates for therapeutic applications. Ther. Deliv. 4 (7); 791-809. The one or more nucleic acid sequences encoding the fusion polypeptides provided herein can be delivered to the cell by any method discussed above or known in the art.

In some embodiments of any of the aspects, the vectors provided herein comprise a nucleic acid modification by methods known in the art. In some embodiments, the cell can be genetically manipulated to express one or more vectors, each encoding one or more vesicle targeting domains and/or one or more signaling domains and one or more linkers. In some embodiments, the cell can be genetically manipulated to express one or more fusion polypeptides, each fusion polypeptide comprising one or more vesicle targeting domain, one or more signaling effector domain (e.g., a POI or fragment thereof), and one or more linker. In certain embodiments, the population of cells has been genetically manipulated. This includes, for example, knockout (KO) or transgenic (TG) cell lines, wherein an endogenous gene has been removed and/or an exogenous introduced in a stable, persistent manner. In certain embodiments, this further includes transient knockdown of one or more genes and associated coding and non-coding transcripts within the population of cells, via any number of methods known in the art, such as introduction of dsRNA, siRNA, microRNA, etc. This further includes transient expression of one or more genes and associated coding and non-coding transcripts within the population of cells, via any number of methods known in the art, such as introduction of a vector, plasmid, artificial plasmid, replicative and/or non-replicative virus, etc.

In certain embodiments the cell population has been manipulated to knock-out the expression of one or more endogenous gene sequences that encode for metalloendopeptidases. In certain embodiments the cell population has been manipulated to knockout the expression of one or more endogenous gene sequences that code for metalloproteinases. In certain embodiments the cell population has been manipulated to knockout the expression of one or more endogenous gene sequences that encode for a disintegrin and metalloproteinase (ADAM). For example, the cell population can be manipulated to knock out the expression of one or more gene sequences that encode for ADAM1, ADAM2, ADAM7, ADAM8, ADAM9, ADAM10, ADAM11, ADAM12, ADAM15, ADAM17, ADAM18, ADAM19, ADAM20, ADAM21, ADAM22, ADAM23, ADAM28, ADAM29, ADAM30, ADAM33, or the like as known by one of ordinary skill in the art.

In certain embodiments the cell population has been manipulated to knockout the expression of one or more endogenous genes that encode for enzymes that hydrolyze the inositol phosphate linkage in proteins anchored by phosphatidylinositol glycans, thereby preventing the release of proteins attached to the plasma membrane via GPI anchors. For example, the cell population can be manipulated to knock of the expression of phosphatidylinositol-glycan-specific phospholipase D (GPLD1).

In certain embodiments, the population of cells has been genetically manipulated. This includes, for example, knock-in of an exogenous genetic sequence, wherein the exogenous genetic sequence is expressed in a stable, persistent manner. In certain embodiments, the cell population has been manipulated to knock-in recombinase recognition sequences (e.g., FRT), transgenic reporters such as antibiotic resistance genes, fluorescent or enzymatic reporter genes, etc. or the like.

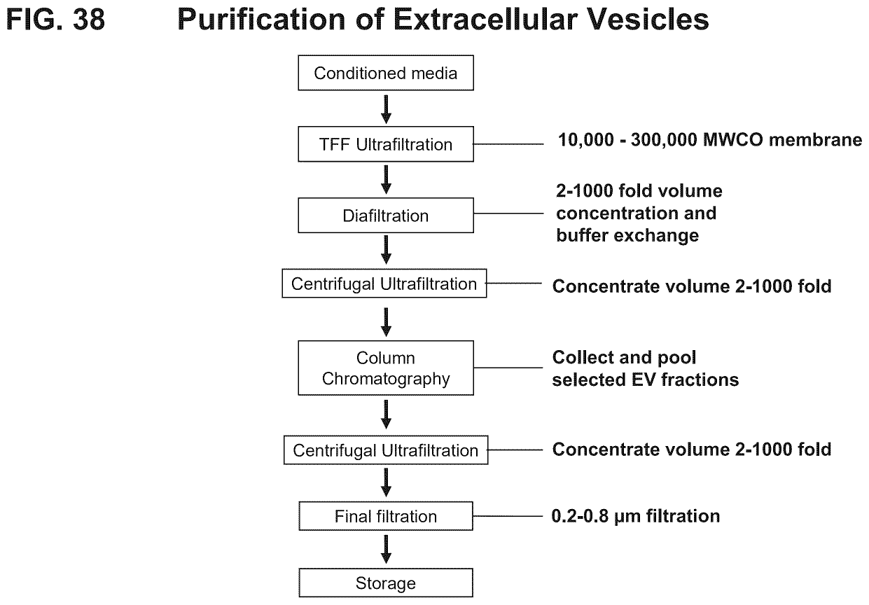

In some embodiments, the method comprises a step of isolating/purifying the engineered extracellular vesicles provided herein. Particulates within the medium are removed by a series of specific centrifugation steps and the media is filtered. The general method of isolating/purifying extracellular vesicles as provided herein is depicted in FIG. 38. Methods of isolating and purifying the extracellular vesicles and exosomes are known in the art and further described, e.g., in Whitford W, Guterstam P. Exosome manufacturing status. Future Med Chem. 2019 May; 11 (10): 1225-1236. doi: 10.4155/fmc-2018-0417. PMID: 31280675, Patel D B, Santoro M, Born L J, Fisher J P, Jay S M. Towards rationally designed biomanufacturing of therapeutic extracellular vesicles: impact of the bioproduction microenvironment. Biotechnol Adv. 2018 December; 36 (8): 2051-2059. doi: 10.1016/j.biotechadv.2018.09.001. Epub 2018 Sep. 12. PMID: 30218694; PMCID: PMC6250573, Ng K S, Smith J A, McAteer M P, Mead B E, Ware J, Jackson F O, Carter A, Ferreira L, Bure K, Rowley J A, Reeve B, Brindley D A, Karp J M. Bioprocess decision support tool for scalable manufacture of extracellular vesicles. Biotechnol Bioeng. 2019 February; 116 (2): 307-319. doi: 10.1002/bit.26809. Epub 2018 Nov. 8. PMID: 30063243; PMCID: PMC6322973, Paganini C, Capasso Palmiero U, Pocsfalvi G, Touzet N, Bongiovanni A, Arosio P. Scalable Production and Isolation of Extracellular Vesicles: Available Sources and Lessons from Current Industrial Bioprocesses. Biotechnol J. 2019 October; 14 (10): e1800528. doi: 10.1002/biot.201800528. Epub 2019 Jul. 8. PMID: 31140717, which are incorporated herein by reference in their entireties.

In some embodiments, isolating the plurality of engineered EVs includes precipitation, centrifugation, filtration, centrifugal ultrafiltration, immuno-separation, tangential flow, liquid chromatography, and/or flow fractionation. For example, differential ultracentrifugation has become a technique wherein secreted exosomes are isolated from the supernatants of cultured cells. This approach allows for separation of exosomes from non-membranous particles, by exploiting their relatively low buoyant density. Size exclusion chromatography allows for extracellular vesicle separation from biochemically similar, but biophysically different microvesicles, which possess larger diameters of up to 1,000 nm. Differences in floatation velocity or sedimentation velocity further allows for separation of differentially sized exosomes. In general, exosome sizes will possess a diameter ranging from 30-300 nm, including sizes of 30-150 nm. Further purification may rely on specific properties of the particular engineered extracellular vesicles of interest. This includes, for example, use of immunoadsorption with a protein of interest to select specific vesicles with exoplasmic or outward orientations. This includes, for example, use of adsorption methods as known by one of ordinary skill in the art, for example, adsorption of a specific exoplasmic protein of interest or fusion polypeptide to select for specific extracellular vesicles displaying said exoplasmic protein of interest or fusion polypeptide.

Among current methods (differential centrifugation, discontinuous density gradients, immunoaffinity, ultrafiltration and liquid chromatography (e.g., fast protein liquid chromatography (FPLC)), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/ml) or application of a discrete sugar cushion in preparation.

Ultrafiltration can be used to purify exosomes without compromising their biological activity. Membranes with different pore sizes-such as 10 kDa molecular weight cut-off (MWCO), or 30 kDa MWCO, or 50 KDa MWCO, or 100 kDa MWCO, or 200 kDa MWCO, or 300 kDa MWCO and gel filtration may be used to eliminate particles smaller than extracellular vesicle, for example to exchange buffer conditions to a desired pH and concentration of solutes (i.e., salts). Currently available tangential flow filtration (TFF) systems are scalable (to >10,000 L), allowing one to not only purify, but concentrate the exosome fractions, and such approaches are less time consuming than differential centrifugation. Liquid Chromatography can also be used to purify exosomes to homogeneously sized particles and preserve their biological activity as the preparation is maintained at a physiological pH and salt concentration.

Other chemical methods have exploited differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to precipitate a population of extracellular vesicles quickly and rapidly (e.g., engineered extracellular vesicles), although re-suspension of pellets prepared via this technique may be difficult. Flow field-flow fractionation (FIFFF) is an elution-based technique that is used to separate and characterize macromolecules (e.g., proteins) and nano- to micro-sized particles (e.g., organelles and cells) and which has been successfully applied to fractionate extracellular vesicles (e.g., engineered extracellular vesicles) from culture media.

Beyond these techniques relying on general biochemical and biophysical features, focused techniques may be applied to isolated specific exosomes of interest. This includes relying on antibody immunoaffinity to recognizing certain exosome-associated antigens. Conjugation to magnetic beads, chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

In several embodiments, isolating a plurality of extracellular vesicles (e.g., engineered extracellular vesicles) from the population of cells includes centrifugation of the cells and/or media conditioned by the cells. In several embodiments, ultracentrifugation is used. In several embodiments, isolating a plurality of extracellular vesicles (e.g., engineered extracellular vesicles) from the population of cells is via size-exclusion filtration. In other embodiments, isolating a plurality of extracellular vesicles (e.g., engineered extracellular vesicles) from the population of cells includes use of discontinuous density gradients, immunoaffinity, ultrafiltration, tangential flow and/or liquid chromatography.

In certain embodiments, differential ultracentrifugation includes using centrifugal force from 1000-2000×g, 2000-3000×g, 3000-4000×g, 4000-5000×g, 5000×g-6000×g, 6000-7000×g, 7000-8000×g, 8000-9000×g, 9000-10,000×g, to 10,000×g or more to separate larger-sized particles from a plurality of extracellular vesicles (e.g., engineered extracellular vesicles) derived from the cells.

In other embodiments, isolating a plurality of extracellular vesicles (e.g., engineered extracellular vesicles) from the population of cells includes use of filtration or ultrafiltration. In certain embodiments, a size exclusion membrane with different pore sizes is used. For example, a size exclusion membrane can include use of a filter with a pore size of 0.1-0.5 micron (μm), 0.5-1.0 μm, 1-2.5 μm, 2.5-5 μm, 5 or more μm. In certain embodiments, the pore size is about 0.2 μm. In certain embodiments, filtration or ultrafiltration includes size exclusion ranging from 100-500 Daltons (Da), 500-1 kDa, 1-2 kDa, 2-5 kDa, 5-10 kDa, 10-25 kDa, 25-50 kDa, 50-100 kDa, 100-250 kDa, 250-500 kDa, 500 or more kDa (kilo Daltons). In certain embodiments, the size exclusion is for about 2-5 kDa. In certain embodiments, the size exclusion is for about 3 kDa. In other embodiments, filtration or ultrafiltration includes size exclusion includes use of hollow fiber membranes capable of isolating particles ranging from 100-500 Da, 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In other embodiments, a molecular weight cut-off (MWCO) gel filtration capable of isolating particles ranging from 100-500 Da, 500-1 kDa, 1-2 kDa, 2-5 kDa, 5-10 kDa, 10-25 kDa, 25-50 kDa, 50-100 kDa, 100-250 kDa, 250-500 kDa, 500 or more kDa. In certain embodiments, the size exclusion is for about 2-5 kDa. In certain embodiments, the size exclusion is for about 3 kDa. In various embodiments, such systems are used in combination with variable fluid flow systems. In certain embodiments, a size exclusion membrane with different pore sizes is used to purify extracellular vesicles from a solution comprising undesirable proteins or nucleic acids.

In other embodiments, isolating a plurality of extracellular vesicles (e.g., engineered extracellular vesicles) from the population of cells includes use of tangential flow filtration (TFF) systems are used purify and/or concentrate the exosome fractions. In other embodiments, isolating a plurality of extracellular vesicles (e.g., engineered extracellular vesicles) from the population of cells includes use of liquid chromatography can also be used to purify extracellular vesicles (e.g., engineered extracellular vesicles) to homogeneously sized particles. In various embodiments, density gradients as used, such as centrifugation in a sucrose density gradient or application of a discrete sugar cushion in preparation.

In other embodiments, isolating a plurality of extracellular vesicles (e.g., engineered extracellular vesicles) from the population of cells includes use of a precipitation reagent. For example, a precipitation reagent, ExoQuick®, can be added to conditioned cell media to precipitate a population of extracellular vesicles quickly and rapidly (e.g., engineered extracellular vesicles). In other embodiments, isolating a plurality of extracellular vesicles (e.g., engineered extracellular vesicles) from the population of cells includes use of volume-excluding polymers (e.g., polyethylene glycols (PEGs)) are used. In another embodiment, isolating a plurality of extracellular vesicles (e.g., engineered extracellular vesicles) from the population of cells includes use of flow field-flow fractionation (FIFFF), an elution-based technique.

In certain embodiments, isolating a plurality of extracellular vesicles (e.g., engineered extracellular vesicles) from the population of cells includes use of one or more capture agents to isolate one or more extracellular vesicles (e.g., engineered extracellular vesicles) possessing specific biomarkers or containing particular biological molecules. In one embodiment, one or more capture agents include at least one antibody. For example, antibody immunoaffinity recognizing exosome-associated antigens is used to capture specific extracellular vesicles (e.g., engineered extracellular vesicles). In other embodiments, the at least one antibody are conjugated to a fixed surface, such as magnetic beads, chromatography matrices, plates, or microfluidic devices, thereby allowing isolation of the specific exosome populations of interest. In other embodiments, isolating a plurality of extracellular vesicles (e.g., engineered extracellular vesicles) from the population of cells includes use of one or more capture agents that is not an antibody. This includes, for example, use of a “bait” molecule presenting an antigenic feature complementary to a corresponding molecule of interest on the exosome surface, such as a receptor or other coupling molecule. In one embodiment, the non-antibody capture agent is a lectin capable of binding to polysaccharide residues on the exosome surface.

In other embodiments, isolating a plurality of extracellular vesicles (e.g., engineered extracellular vesicles) from the population of cells includes use of ion exchange chromatography. In other embodiments, isolating a plurality of extracellular vesicles (e.g., engineered extracellular vesicles) from the population of cells includes use of anion exchange chromatography. In other embodiments, isolating a plurality of extracellular vesicles (e.g., engineered extracellular vesicles) from the population of cells includes use of cation exchange chromatography. In certain embodiments, ion exchange chromatography comprises a chromatography resin with a functional group selected from the group consisting of diethylaminoethyl (DEAE), quaternary aminoethyl (QAE), quaternary ammonium (Q), carboxymethyl (CM), sulfopropyl (SP), or methyl sulfate(S). In certain embodiments, ion exchange chromatography comprises a chromatography resin which may have properties of a weak acid, strong acid, weak base, or strong basic. In certain embodiments, ion exchange chromatography comprises a chromatography selected from the group consisting of DEAE cellulose, DEAE Sephadex, Mono Q, Mini Q, HiTrap Capto, Capto Core 700, HiPrep Q, QAE Sephadex, Q Sepharose, CM Cellulose, SP Sepharose, SOURCE S, EAH-Sepharose, sulfoxyethyl cellulose, CM Sephadex, or CM Sepharose. Isolating a plurality of extracellular vesicles (e.g., engineered extracellular vesicles) can be prepared by any of a variety of ion exchange chromatography techniques that are known in the art.

In other embodiments, isolating a plurality of extracellular vesicles (e.g., engineered extracellular vesicles) from the population of cells includes use of a nuclease enzyme (e.g., a DNase or RNase). For example, a working concentration of Benzonase® nuclease may be added to an extracellular vesicle sample preparation in the presence of a divalent cation, for example 1-2 mM Mg²⁺, 2-5 mM Mg²⁺, 10-20 mM Mg²⁺, 20-50 mM Mg²⁺, 50-100 mM Mg²⁺, or more than 100 mM Mg²⁺.

Following isolation and purification of the engineered EVs provided herein, EVs can be further evaluated for the desired structural and functional properties by methods known in the art. For example, the engineered exosomes provided herein can be assayed for functional activity on a target cell using a cell-based bioassays (e.g., those commercially available, Promega DiscoverX®), ligand-receptor binding assays, vesicle flow cytometric assays, enzyme-linked immunosorbent assays, tunable resistive pulse sensing (TRPS), nanoparticle tracking analysis (NTA), surface plasmon resonance (SSPR), nucleotide sequencing, lipidomics, proteomics, colorimetric assays, fluorescence assays, luminescence assays, immunoblotting, radioimmunoassays, electron microscopy, or EV automated analysis (e.g., ExoView®, Unchained Labs). Additional methods of characterizing EVs are found, e.g., in Zhang Y, Bi J, Huang J, Tang Y, Du S, Li P. Exosome: A Review of Its Classification, Isolation Techniques, Storage, Diagnostic and Targeted Therapy Applications. Int J Nanomedicine. 2020 Sep. 22; 15:6917-6934. doi: 10.2147/IJN.S264498. PMID: 33061359; PMCID: PMC7519827, Kluszczyńska K, Czernek L, Cypryk W, Pęczek Ł, Düchler M. Methods for the Determination of the Purity of Exosomes. Curr Pharm Des. 2019; 25 (42): 4464-4485. doi: 10.2174/1381612825666191206162712. PMID: 31808383, Nolan J P, Duggan E. Analysis of Individual Extracellular Vesicles by Flow Cytometry. Methods Mol Biol. 2018; 1678:79-92. doi: 10.1007/978-1-4939-7346-0_5. PMID: 29071676.; Doyle L M, Wang M Z. Overview of Extracellular Vesicles, Their Origin, Composition, Purpose, and Methods for Exosome Isolation and Analysis. Cells. 2019 Jul. 15; 8 (7): 727. doi: 10.3390/cells8070727. PMID: 31311206; PMCID: PMC6678302, Pugholm L H, Revenfeld A L, Søndergaard EK, Jørgensen M M. Antibody-Based Assays for Phenotyping of Extracellular Vesicles. Biomed Res Int. 2015; 2015:524817. doi: 10.1155/2015/524817. Epub 2015 Dec. 3. PMID: 26770974; PMCID: PMC4681819, Shao H, Im H, Castro C M, Breakefield X, Weissleder R, Lee H. New Technologies for Analysis of Extracellular Vesicles. Chem Rev. 2018 Feb. 28; 118 (4): 1917-1950. doi: 10.1021/acs.chemrev.7b00534. Epub 2018 Jan. 31. PMID: 29384376; PMCID: PMC6029891, which are incorporated herein by reference in their entireties.

### Pharmaceutical Compositions

Provided herein are compositions comprising the engineered extracellular vesicles provided herein.

In one aspect, provided herein is a composition comprising: a plurality of the engineered extracellular vesicles provided herein. In some embodiments of any of the aspects, the compositions and engineered EVs provided herein further comprise a pharmaceutically acceptable carrier.

For clinical use of the methods and compositions described herein, administration of the engineered extracellular vesicles provided herein can include formulation into pharmaceutical compositions or pharmaceutical formulations for parenteral administration, e.g., intravenous; mucosal, e.g., intranasal; ocular, or other mode of administration. In some embodiments, the engineered EVs described herein can be administered along with any pharmaceutically acceptable carrier compound, material, or composition which results in an effective treatment in the subject. Thus, a pharmaceutical formulation for use in the methods described herein can contain the engineered EVs described herein in combination with one or more pharmaceutically acceptable ingredients. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an engineered EV as described herein. Each carrier must be “acceptable” in the sense of being compatible with the other ingredients of the formulation and not injurious to the patient. The engineered EVs provided herein can be formulated for administration of the compound to a subject in solid, liquid, or gel form, including those adapted for the following: (1) parenteral administration, for example, by subcutaneous, intramuscular, intravenous or epidural injection as, for example, a sterile solution or suspension, or sustained-release formulation; (2) transdermally; (3) transmucosally; (4) via bronchoalveolar lavage.

In some embodiments, the compositions described herein comprise a particle or polymer-based vehicle. Exemplary particle or polymer-based vehicles include, but are not limited to, nanoparticles, microparticles, polymer microspheres, or polymer-drug conjugates.

In one embodiment of any of the aspects, the compositions described herein further comprise a lipid vehicle. Exemplary lipid vehicles include, but are not limited to, liposomes, phospholipids, micelles, lipid emulsions, and lipid-drug complexes.

Formulations can be adapted for delivery to the airway, e.g., to address respiratory inflammation. Such formulations can be adapted for delivery as an aerosol, e.g., for inhalation. In some embodiments, the compositions described herein are formulated for aerosol administration, nebulizer administration, tracheal lavage administration, or for a pulmonary delivery device.

As used herein, the term “pulmonary delivery device” refers to a device used to deliver a therapeutic dose of a composition of the present invention to the respiratory system including, but not limited to, a nebulizer, metered-dose inhaler, or dry powder inhaler.

Examples of nebulizers include, but are not limited to, soft mist inhalers (for example Respimat® Boehringer Ingelheim) jet nebulizers (use compressed gas or air), ultrasonic nebulizers (produce aerosols using a piezoelectric crystal vibrating at high frequencies), and vibrating mesh nebulizers.

For use as aerosols, the compositions described herein can be prepared in a solution or suspension and may be packaged in a pressurized aerosol container together with suitable propellants, for example, hydrocarbon propellants like propane, butane, or isobutane with conventional excipients.

The engineered EVs provided herein can also be administered in a non-pressurized form such as in a nebulizer or atomizer that reduces a liquid to a fine spray. Preferably, by such nebulization small liquid droplets of uniform size are produced from a larger body of liquid in a controlled manner. Nebulization can be achieved by any suitable method, device or system therefor, including by using many nebulizers known and marketed today. For example, an AEROMIST™ pneumatic nebulizer available from Inhalation Plastic, Inc. of Niles, Ill.

In some embodiments, the engineered EVs described herein can be formulated with micro-emulsification technology. Micro-emulsification technology can improve bioavailability of some lipophilic (water insoluble) pharmaceutical agents. Examples include Trimetrine (Dordunoo, S. K., et al., Drug Development and Industrial Pharmacy, 17 (12), 1685-1713, 1991 and REV 5901 (Sheen, P. C., et al., J Pharm Sci 80 (7), 712-714, 1991), the contents of which are incorporated herein by reference in their entirety. Among other things, micro-emulsification provides enhanced bioavailability by preferentially directing absorption to the lymphatic system instead of the circulatory system, which thereby bypasses the liver, and prevents destruction of the cell-based compositions in the hepatobiliary circulation.

The engineered EVs described herein can be formulated with an amphiphilic carrier. Amphiphilic carriers are saturated and monounsaturated polyethylene glycolyzed fatty acid glycerides, such as those obtained from fully or partially hydrogenated various vegetable oils. Such oils may advantageously consist of tri-, di-, and mono-fatty acid glycerides and di- and mono-polyethylene glycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

The engineered EV compositions provided herein can be formulated with hydrophilic polymers. Hydrophilic polymers are water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a pharmaceutical composition as described herein comprises an engineered extracellular vesicle and a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho) esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, a pharmaceutical composition described herein is formulated as a liposome. Liposomes can be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic D D, Liposomes from physics to applications, Elsevier Science Publishers BV, Amsterdam, 1993, the contents of which are incorporated herein by reference in their entireties.

Therapeutic formulations of the engineered EV compositions as described herein can be prepared for storage by with optional pharmaceutically acceptable carriers, excipients, or stabilizers (Remington's Pharmaceutical Sciences 16th edition, Osol, A. Ed. (1980) the contents of which is incorporated herein by reference in its entirety.), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Vaccine or other pharmaceutical compositions comprising an engineered EV composition as described herein can contain a pharmaceutically acceptable salt, typically, e.g., sodium chloride, and preferably at about physiological concentrations. The formulations of the vaccine or other pharmaceutical compositions described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. The formulations of the vaccine or other pharmaceutical compositions described herein can include a pharmaceutically acceptable surfactant at a concentration of 0.005 to 0.02%.

Therapeutic pharmaceutical compositions described herein can also contain more than one active compound as necessary for the particular indication being treated, preferably those with complementary activities that do not adversely affect each other.

In some embodiments in which the engineered EVs are formulated for use in or with a vaccine, the vaccine composition can be formulated with the engineered EVs as an adjuvant. In other embodiments the vaccine composition can be formulated with the engineered EVs and an additional adjuvant, e.g., as known in the art.

As used herein in the context of immunization, immune response and vaccination, the term “adjuvant” refers to any substance than when used in combination with a specific antigen produces a more robust immune response than the antigen alone. When incorporated into a vaccine formulation, an adjuvant acts generally to accelerate, prolong, or enhance the quality of specific immune responses to the vaccine antigen(s). Adjuvants typically promote the accumulation and/or activation of accessory cells or factors to enhance antigen-specific immune responses and thereby enhance the efficacy of vaccines, i.e., antigen-containing or encoding compositions used to induce protective immunity against the antigen.

Adjuvants, in general, include adjuvants that create a depot effect, immune-stimulating adjuvants, and adjuvants that create a depot effect and stimulate the immune system. An adjuvant that creates a depot effect is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX™ (an oil-in-water emulsion containing a stabilizing detergent and a micelle-forming agent; IDEC Pharmaceuticals Corporation, San Diego, Calif.).

An immune-stimulating adjuvant is an adjuvant that causes activation of a cell of the immune system. It may, for instance, cause an immune cell to produce and secrete cytokines and interferons. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly [di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma S A, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The active ingredients of the pharmaceutical compositions described herein including the engineered extracellular vesicle can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980) which is incorporated herein by reference in its entirety.

In some embodiments, sustained-release preparations can be used. Suitable examples of sustained-release preparations include semipermeable matrices of solid hydrophobic polymers containing a composition described herein in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-3-hydroxybutyric acid. While polymers such as ethylene-vinyl acetate and lactic acid-glycolic acid enable release of molecules for over 100 days, certain hydrogels release proteins for shorter time periods. When encapsulated, the composition can remain in the body for a long time (e.g., up to about 1 hour, between 1-12 hours, 12-24 hours, 24 hours to 2 days, 2-3 days, 3-4 days, 4-5 days, 5-6 days, 6-7 days, 1-2 weeks, 3-4 weeks, 4 weeks to 2 months, 2-3 months, 3-4 months, 4-5 months, 5-6 months, or more than 6 months, or a variation thereof), denature, or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S— bond formation through thio-disulfide interchange, stabilization can be achieved by modifying sulfhydryl residues, lyophilizing from acidic solutions, controlling moisture content, using appropriate additives, and developing specific polymer matrix compositions.

### Administration, Dosing, Efficacy

The engineered EV compositions, pharmaceutical compositions, or vaccine compositions described herein can be formulated, dosed, and administered in a fashion consistent with good medical practice. Factors for consideration in this context include the particular disorder being treated, the particular subject being treated, the clinical condition of the individual subject, the cause of the disorder, the site of delivery of the vaccine composition, the method of administration, the scheduling of administration, and other factors known to medical practitioners.

Generally, application of engineered extracellular vesicles as therapy will take into account similar parameters as other therapeutic strategies, including concentration, timing of delivery, and sustained bioavailability at injury/disease site. Engineered extracellular vesicle can be delivered via a number of routes: intravenous, intracoronary, and intramyocardial. Extracellular vesicles (e.g., engineered extracellular vesicle or exosomes), also allow for new delivery routes that were previously infeasible for cell therapy, such as inhalation or injection. These various approaches are described below, including injection, topical application, enteral administration, and pulmonary delivery.

The engineered EV compositions provided herein can be administered to a subject in need thereof by any appropriate route which results in an effective treatment in the subject. As used herein, the terms “administering,” and “introducing” are used interchangeably and refer to the placement of a composition provided herein into a subject by a method or route which results in at least partial localization of such compositions at a desired site, such as a site of inflammation or a tumor, such that a desired effect(s) is produced. The compositions can be administered to a subject by any mode of administration that delivers the composition systemically or to a desired surface or target, and can include, but is not limited to, injection, infusion, instillation, and inhalation administration. To the extent that the composition can be protected from inactivation in the gut, oral administration forms are also contemplated. “Injection” includes, without limitation, intravenous, intramuscular, intra-arterial, intrathecal, intraventricular, intracapsular, intraorbital, retro-orbital, intravitreal, intraocular, intracardiac, intradermal, intraperitoneal, transtracheal, subcutaneous, subcuticular, intraarticular, sub capsular, subarachnoid, intraspinal, intracerebral, intratarsal, and intrasternal, intratumoral injection, and infusion or the like as known in the art.

A therapeutic does of the present invention may be delivered to a patient by way of controlled release, for example but not limited to, implantable pump and implantable cannulas to provide continuous access to the venous or arterial system.

Topical application refers to applying or spreading a composition of the present invention onto surfaces on or in the body, both internally and/or externally, in a therapeutically effective amount for local and/or systemic treatment. Topical application may be epicutaneuos wherein a composition of the present invention may be directly applied onto a localized surface of the skin or mucous membranes. Topical application may include transdermal application wherein a composition of the present invention may be absorbed into the body to obtain systemic delivery and systemic distribution. Topical application formulations may include, but are not limited to, creams, foams, gels, lotions, solutions, ointments, dermal patch, transdermal patches, powder, solid, sponge, tape, vapor, paste, film, liposomes, balm, shampoo, spray, or tincture or the like or a combination thereof. A therapeutic dose of a composition of the present invention may be delivered vaginally (for example a vaginal suppository, vaginal ring, douche, intrauterine device, intravesical infusion, and the like) or urethra or the like or a combination thereof.

Enteral administration refers to a composition of the present invention administered via the gastrointestinal tract in a therapeutically effective amount for local or systemic treatment. Enteral administration may include, but is not limited to, delivery of a composition of the present invention via the mouth, sublingual, esophagus, gastric (for example the stomach), small intestines, large intestines, or rectum. Oral delivery of the present invention may include, but is not limited to, the use of a capsule, pastille, pill, tablet, solution, gel, suspension, emulsion, syrup, elixir, tincture, mouthwash, lozenges, chewing gum, lollipop, cream, foam, solution, powder, solid, vapor, liposomes, spray, or tincture osmotic-controlled release oral delivery system, or the like. Gastric delivery may involve the use of a tube or nasal passage that leads directly to the stomach, for example, a percutaneous endoscopic gastrostomy tube. Gastric delivery may involve direct injection made through the abdominal wall. Rectal delivery may involve, but is not limited to, the use of a suppository, ointment, enema, murphy drip, or the like. A therapeutic does of the present invention may be delivered to a patient by way of controlled release, for example but not limited to, controlled release drug delivery pellet or pill.

Inhalation (i.e., pulmonary delivery, pulmonary administration refers to delivery to the respiratory system through the respiratory route, including but not limited to, intranasal administration, oral administration, and oral inhalative administration (e.g. intratracheal instillation and intratracheal inhalation) of a therapeutically effective amount for local or systemic treatment. Pulmonary delivery of a therapeutically effective amount of a composition of the present invention may be achieved by dispersion, for example by using a syringe. Pulmonary delivery of a composition of the present invention may be achieved by aerosol administration, wherein aerosol administration may deposit a therapeutically effective amount of the present invention by gravitational sedimentation, inertial impaction, or diffusion.

Intravenous delivery technique can occur through a peripheral or central venous catheter. As the simplest delivery mode, this technique avoids the risk of an invasive procedure. However, intravenous may be regarded as a comparatively inefficient and less localized delivery method, as a high percentage of infused cell exosomes may become sequestered in organs such as the lung, liver, or spleen. Such sequestration may result in few or no cellular exosomes reaching broader circulation or have unintended systemic effects following their distribution.

In certain embodiments, administration can include delivery to a tissue or organ site that is the same as the site of diseased and/or dysfunctional tissue. In certain embodiments, administration can include delivery to a tissue or organ site that is different from the site or diseased and/or dysfunctional tissue. In certain embodiments, the delivery is via inhalation or oral administration. In various embodiments, administration of engineered extracellular vesicles can include combinations of multiple delivery techniques.

In some embodiments, the compositions described herein are administered by aerosol administration, nebulizer administration, or tracheal lavage administration.

The term “effective amount” as used herein refers to the amount of an engineered EV composition needed to alleviate or prevent at least one or more symptom of a disease or disorder (e.g., autoimmune disease or cancer), and relates to a sufficient amount of pharmacological composition to provide the desired effect, e.g., reduce the pathology, or any symptom associated with or caused by a disease. The term “therapeutically effective amount” therefore refers to an amount of an engineered EV composition or vaccine composition described herein using the methods as disclosed herein, that is sufficient to affect a particular disease state when administered to a typical subject. An effective amount as used herein would also include an amount sufficient to delay the development of a symptom of the disease, alter the course of a symptom disease (for example, but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the engineered EVs or fusion polypeptides provided herein), which achieves a half-maximal inhibition of symptoms) as determined in cell culture, or in an appropriate animal model. Levels of therapeutic engineered EVs in plasma can be measured, for example, by high performance liquid chromatography, enzyme linked immunosorbent assay (ELISA), flow cytometry, FACS sorting, western blot, mass spectroscopy, tunable resistive pulse sensing, ExoView® (Unchained Labs), qRT-PCR, next generation sequencing (NGS), or by any analysis technique known by one of ordinary skill in the art. The effects of any particular dosage can be monitored by a suitable bioassay. The dosage can be determined by a physician and adjusted, as necessary, to suit observed effects of the treatment.

The engineered EV compositions, pharmaceutical compositions, or vaccine compositions described herein can be formulated, in some embodiments, with one or more additional therapeutic agents currently used to prevent or treat the infection, for example. The effective amount of such other agents depends on the amount of an engineered EV in the formulation, the type of disorder or treatment, and other factors discussed above. These are generally used in the same dosages and with administration routes as used herein before or about from 1 to 99% of the heretofore employed dosages.

The dosage ranges for the pharmaceutical compositions of engineered extracellular vesicles described herein depend upon the potency and encompass amounts large enough to produce the desired effect. The dosage should not be so large as to cause unacceptable adverse side effects. Generally, the dosage will vary with the age, condition, health, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 μg/kg body weight to 100 μg/kg body weight. Alternatively, the dose range can be titrated to maintain serum levels between 0.1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the infection is treated, as measured by the methods described above or known in the art. However, other dosage regimens can be useful.

In various embodiments, the quantities of engineered extracellular vesicles that are administered to achieve these effects range from 1×10⁶ to 1×10⁷, 1×10⁷ to 1×10⁸, 1×10⁸ to 1×10⁹, 1×10⁹ to 1×10¹⁰, 1×10¹⁰ to 1×10¹¹, 1×10¹¹ to 1×10¹², 1×10¹² to 1×10¹³, 1×10¹³ to 1×10¹⁴, 1×10¹⁴ to 1×10¹⁵, 1×10¹⁵ or more engineered extracellular vesicles. In other embodiments, the numbers of engineered extracellular vesicles are relative to the number of cells used in a clinically relevant dose for a cell-therapy method. For example, defining an effective dose range, dosing regimen and route of administration, may be guided by studies using fluorescently labeled engineered extracellular vesicles, and measuring target tissue retention, which can be >10×, >50×, or >100× background, as measured 5, 10, 15, 30, or 30 or more min as a screening criterion. In certain embodiments, >100× background measured at 30 mins is a baseline measurement for a low and high dose that is then assess for safety and bioactivity (e.g., using MRI endpoints: scar size, global and regional function of the target organ being treated). In various embodiments, single doses are compared to two, three, four, four or more sequentially applied doses. In various embodiments, the repeated or sequentially applied doses are provided for treatment of an acute disease and/or condition. In various embodiments, the repeated or sequentially applied doses are provided for treatment of a chronic disease and/or condition. In other embodiments, administration of the plurality of engineered extracellular vesicles is adjunctive to standard therapy.

In other embodiments, administering a composition includes 1×10¹⁰ or more engineered extracellular vesicles in a single dose. In various embodiments, exosome quantity may be defined by protein quantity, such as dosages including 1-10, 10-25, 25-50, 50-75, 75-100, or 100 or more mg exosome protein. In other embodiments, a single dose is administered multiple times to the subject. In other embodiments, administering a composition consists of one or more of: injection, topical administration, enteral, intravenous, intra-arterial, or inhalation.

In various embodiments, engineered extracellular vesicles quantity may be defined by protein quantity, such as dosages including 1-10, 10-25, 25-50, 50-75, 75-100, or 100 or more mg exosome protein. In various embodiments, administering a composition includes multiple dosages of the engineered extracellular vesicles. In various embodiments, the repeated or sequentially applied doses are provided for treatment of an acute disease and/or condition. In various embodiments, the repeated or sequentially applied doses are provided for treatment of a chronic disease and/or condition.

In other embodiments, administering a composition including a plurality of engineered extracellular vesicles to the subject is adjunctive to standard therapy.

The duration of a therapy using the methods described herein will continue for as long as medically indicated or until a desired therapeutic effect (e.g., those described herein) is achieved. In certain embodiments, the administration of the vaccine composition described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

As will be appreciated by one of skill in the art, appropriate dosing regimens for a given composition of engineered extracellular vesicles can comprise a single administration/immunization or multiple ones. Subsequent doses may be given repeatedly at time periods, for example, about two weeks or greater up through the entirety of a subject's life, e.g., to provide a sustained preventative effect. Subsequent doses can be spaced, for example, about two weeks, about three weeks, about four weeks, about one month, about two months, about three months, about four months, about five months, about six months, about seven months, about eight months, about nine months, about ten months, about eleven months, or about one year after a primary immunization.

The precise dose to be employed in the formulation will also depend on the route of administration and should be decided according to the judgment of the practitioner and each patient's circumstances. Ultimately, the practitioner or physician will decide the amount of the engineered EV or composition thereof to administer to particular subjects.

### Methods of Modulating Inflammation and Autoimmune Diseases

The engineered extracellular vesicles and compositions thereof provided herein can be deployed in a therapeutic strategy against virtually any injury/disease, as providing a platform for altering biological signaling. This includes, for example, inflammation and immune signaling, which plays a role in virtually all injuries and diseases in living organisms.

Thus, described herein is a method of modulating inflammation, including selecting a subject afflicted with an inflammatory related disease and/or condition; and administering to the subject a composition including a plurality of engineered extracellular vesicles to the subject, wherein administration of the composition modulates inflammation.

As used herein, the term “inflammation” or “inflamed” refers to activation or recruitment of the immune system or immune cells (e.g., T cells, B cells, macrophages). A tissue that has inflammation can become reddened, white, swollen, hot, painful, sensitivity, exhibit a loss of function, or have a film or mucus. Methods of identifying inflammation are well known in the art. Inflammation typically occurs following injury, infection by a microorganism, exposure to a substance (e.g., a toxin, chemical, or dust) or autoimmune dysfunction. Onset of inflammation may be rapid (e.g., immediately following injury) or slow (e.g., repeated exposure to an irritant such as a chemical over time) with a duration of minutes, hours, days, months, years, or an individual's life.

Inflammation plays a vital role in alerting the immune system of potential danger and damage within a body. Inflammation is necessary to control and repair injury. For example, acute inflammation is a response to physical trauma, infection, and stress. Acute inflammation helps prevent further injury and triggers healing and recovery. Unfortunately, inflammation can become excessive and inappropriately active, lasting beyond the typical recovery time from an injury or infection. Wherein healthy inflammation helps a body respond to injury, chronic inflammation perpetuates injury and may lead to negative consequences to one's health. In particular, autoimmune diseases are chronic diseases from a host's immune system attacking itself, often due to aberrant biological signaling in the host. Restoring normal homeostatic signaling via application of engineered extracellular vesicles, particularly targeting immune checkpoints, represents a highly promising avenue. For example, surface bound immune-checkpoint proteins or fragments thereof may modulate immune cell stimulation and affect suppression or activation of immune cell function when delivered via engineered extracellular vesicles. Injection, inhalation, ingestion, or topical application of engineered extracellular vesicles with surface bound immune-checkpoint proteins or fragments thereof may be used to treat immune, auto-immune, inflammatory, auto-inflammatory conditions or cancer diseases. Examples include chronic obstructive pulmonary disease (COPD) which is an inflammatory, progressive, life-threatening lung disease, psoriasis, a common chronic noncommunicable inflammatory skin disease, arthritis, a debilitating and painful degeneration of joints, among others well-understood to one of skill in the art.

In other embodiments, the inflammatory related disease and/or condition is acute, for example septicemia. In other embodiments, the inflammatory related disease and/or condition is chronic, for example chronic obstructive pulmonary disease. In other embodiments, the inflammatory condition is an autoimmune disease wherein the autoimmune disease and/or condition is one or more of: polymyositis, dermatomyositis, Graves' disease, Hashimoto's thyroiditis, myasthenia gravis, vasculitis, multiple sclerosis, psoriasis, rheumatoid arthritis, psoriatic arthritis, scleroderma, systemic lupus erythematosus, inflammatory bowel disease, Crohn's disease, hyperthyroidism, autoimmune adrenal insufficiency, Sjogren syndrome, type I diabetes mellitus, autoimmune hemolytic anemia, idiopathic thrombocytopenia purpura, myasthenia gravis, ulcerative colitis, uveitis, polyarteritis nodosa, relapsing polychondritis, Behcet's disease, reactive arthritis, ankylosing spondylitis, Guillain-Barre syndrome, or optic neuropathy. In other embodiments, the disease and/or condition is chronic obstructive pulmonary disease, rheumatoid arthritis, uveoretinitis, psoriasis, and eczema. In other embodiments, the disease and/or condition is irritable bowel disease, multiple sclerosis or lupus.

In other embodiments, the inflammatory related disease and/or condition is an ocular disease. As used herein, the terms “ocular disease”, “eye disorder” and “eye disease” are used interchangeably and refer to a disease or disorder that affects the health and/or vision of either one or both eyes or the general area of the eye(s), eye lid(s), or area surrounding or in near proximity to the eye(s). Eye disease may include, but are not limited to, macular degeneration (e.g., age-related macular degeneration), cataracts, diabetic retinopathy, diabetic macular edema, eye floaters, eye flashes, glaucoma, amblyopia, strabismus, retinitis (e.g., CMV retinitis), color blindness, keratoconus, retinal detachment, eyelid twitching, ocular hypertension, blepharitis, uveitis, Bietti's crystalline dystrophy, blepharospasm, cornea and corneal diseases, dry eye, histoplasmosis, macular hole, macular pucker, conjunctivitis, presbyopia, retinoblastoma, retinitis pigmentosa, retinopathy, Stargardt disease, Usher syndrome, uveal Coloma, and vitreous detachment, or the like.

Described herein is a method of administering to a subject an engineered EV or composition thereof as provided herein to the subject having an autoimmune disease, inflammation, inflammatory disease or condition, or cancer. In various embodiments, the method stimulates the subject's T cells or increases the number of T cells in the subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total usage of the compositions and formulations as disclosed herein will be decided by the attending physician within the scope of sound medical judgment. The exact amount required will vary depending on factors such as the type of disease being treated.

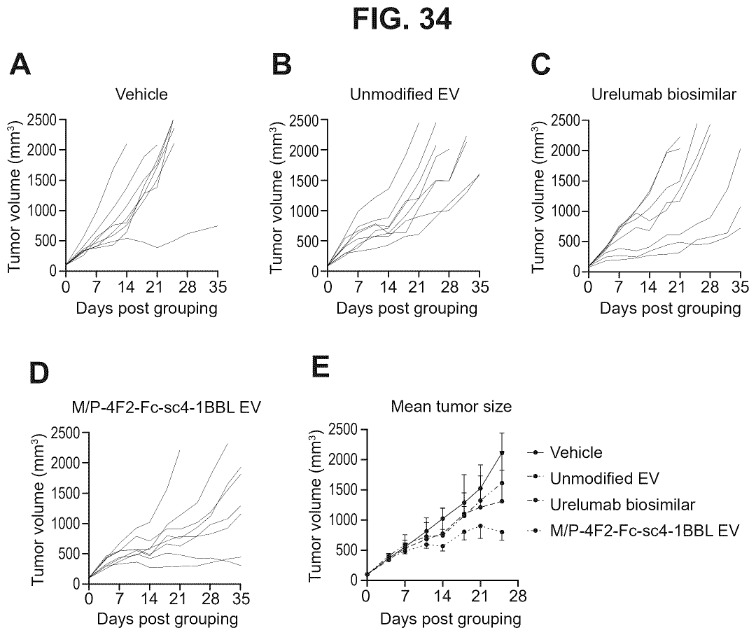

Non-limiting examples of clinical tests that can be used to assess autoimmune diseases, inflammatory conditions, cancer, or inflammation parameters include blood tests, skin biopsy, MRI, eye examination, ocular pressure tests, etc. Where necessary or desired, animal models of injury, auto-immune disease, inflammatory disease, of cancer can be used to gauge the effectiveness of a particular composition as described herein. For example, an MC38 colon carcinoma animal model, as demonstrated in a working example can be used (FIG. 34).

In various embodiments, administration of the plurality of engineered extracellular vesicles alters gene expression in the damaged or dysfunctional tissue, improves viability of the damaged tissue, and/or enhances regeneration or production of new tissue in the individual. In various embodiments, administration of the plurality of engineered extracellular vesicles alters gene expression in the damaged or dysfunctional tissue, improves viability of the damaged tissue, and/or enhances regeneration or production of new tissue in the individual.

In various embodiments, the damaged or dysfunctional tissue is in need of repair, regeneration, or improved function due to an acute event. Acute events include, but are not limited to, trauma such as laceration, crush or impact injury, shock, loss of blood or oxygen flow, infection, chemical or heat exposure, poison or venom exposure, drug overuse or overexposure, and the like. Other sources of damage also include, but are not limited to, injury, age-related degeneration, cancer, and infection. In several embodiments, the regenerative cells used to prepare the engineered EVs provided herein are from the same tissue type as is in need of repair or regeneration. In several other embodiments, the regenerative cells are from a tissue type other than the tissue in need of repair or regeneration. In some embodiments, the engineered EVs provided herein are derived from the subject being treated. In some embodiments, the engineered EVs are derived from a donor subject.

In other embodiments, the damaged or dysfunctional tissue is in need of repair, regeneration, or improved function due to damage from chronic disease.

### Methods of Treating Cancer

Various embodiments provide for a method of stimulating immune cells in a subject having cancer. In various embodiments the method comprises administering an engineered EV of the present invention to the subject. In various embodiments, the subject's T cells increase by about 10%, 20%, 30% or 50%. In various embodiments, the subject's T cells increase by about 60%, 70%, 80%, 90% or 100%. In various embodiments, the subject's T cells increase by at least 100%. In various embodiments, the T cells are CD8+ T cells, cytotoxic T cells, CD4+ T cells, T helper (Th) cells (e.g., Th1, Th2, Th17, Th9, Tfh, Th22), regulatory T cells, memory T cells (e.g., central memory T cells (T_{CM}), effector memory T cells (T_{EM}), terminally differentiated effector memory T cells (T_{EMRA}), tissue resident memory T cells (T_{RM}), virtual memory T cells (T_{VM})), innate-like T cells, natural killer T (NKT) cells, mucosal associated invariant T (MAIT) cells, gamma delta (γδ) T cells, or T cells functioning as antigen-presenting cells (APCs).

Various embodiments provide for a method of treating colon cancer. In various embodiments the method comprises administering an engineered EV of the present invention to the subject having colon cancer.

### Some Selected Definitions

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed, Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, NY 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, NY 2012), provide one skilled in the art with a general guide to many of the terms used in the present application. For references on the preparation and structure of antibodies and fusion polypeptides, see, e.g., Greenfield, Antibodies A Laboratory Manual 2^{nd} ed., Cold Spring Harbor Press (Cold Spring Harbor NY, 2013); Köhler and Milstein, Derivation of specific antibody-producing tissue culture and tumor lines by cell fusion, Eur. J. Immunol. 1976 Jul. 6 (7): 511-9; Queen and Selick, Humanized immunoglobulins, U.S. Pat. No. 5,585,089 (1996 December); and Riechmann et al., Reshaping human antibodies for therapy, Nature 1988 Mar. 24, 332 (6162): 323-7. See also, Kabat et al., Sequences of Proteins of Immunological Interest, 5th Ed. Public Health Service, National Institutes of Health, Bethesda, Md. (1991), Lanzavecchia et al., Eur. J. Immunol. 17, 105 (1987)), Huston et al., Proc. Natl. Acad. Sci. U.S.A., 85, 5879-5883 (1988), Bird et al., Science 242, 423-426 (1988), Brinkman et al. mAbs Vol 9, No. 2, 182-212 (2017), Chothia & Lesk, J. Mol. Biol, 196:901-917 (1987), Chothia et al., Nature 342:877-883 (1989)), Holliger et al. (1993) Proc. Natl. Acad. Sci. USA 90:6444-6448; Poljak (1994) Structure 2:1121-1123); Kontermann and Dubel eds., Antibody Engineering, Springer-Verlag, N.Y. (2001), p. 790 (ISBN 3-540-41354-5, Zapata et al. (1995) Protein Eng. 8 (10): 1057-1062; Morrison, et al., Proc. Natl. Acad. Sci. USA, 81:6851 (1984), U.S. Pat. Nos. 4,816,567, 5,693,780, which are incorporated herein by reference in their entireties.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein, the term “extracellular vesicle” and “vesicle” refer to a particle, wherein the particle comprises a phospholipid bilayer that encloses an internal space and an exterior surface and may or may not be derived from a cell. The size of extracellular vesicles can range between 20 nm to 3 μm in diameter but may be smaller than 20 nm or larger than 3 μm. Examples of extracellular vesicles include, but is not limited to, exosomes (for example small exosomes and large exosomes), ectosomes, macrovesicles, microparticles, apoptotic bodies, vesicular organelles, oncosomes (for examples large oncosomes), exospheres, exomeres, cell derived nanovesicles (CDN) (e.g., by genesis via grating or shearing cells), liposomes or the like known by one of ordinary skill in the art. Extracellular vesicles may originate naturally via known or unknown biosynthetic pathways. Extracellular vesicles may be promoted to originate by using mechanical methods such as cell grating or cell shearing wherein a cell is grated or sheared causing portions or parts of the cell membrane to from vesicles. For example, CDNs may be formed by using mechanical methods such as cell grating or cell shearing wherein a cell is grated or sheared causing portions or parts of the cell membrane to from vesicles. Additional non-limiting examples of mechanical methods that can be used to form cell derived nanovesicles are further described in detail, e.g., Goh, W. J., Zou, S., Ong, W. Y. et al. Bioinspired Cell-Derived Nanovesicles versus Exosomes as Drug Delivery Systems: a Cost-Effective Alternative. Sci Rep 7, 14322 (2017). doi.org/10.1038/s41598-017-14725-x, the contents of which are incorporated herein by reference in their entireties. In some embodiments, vesicles may be prepared artificially via known synthetic pathways, for example vesicles may be lipid nanoparticles or liposomes.

As used herein, the term “engineered extracellular vesicle” refers to an extracellular vesicle designed to comprise one or more engineered fusion polypeptides. In some embodiments “engineered extracellular vesicle” refers to an extracellular vesicle that displays one or more engineered fusion polypeptides, wherein the one or more fusion polypeptides is an agonist to a target receptor. In some embodiments “engineered extracellular vesicle” refers to an extracellular vesicle that displays one or more engineered fusion polypeptides, wherein the one or more fusion polypeptides is an agonist to a target receptor, and wherein the agonistic fusion polypeptide induces signaling of the target receptor through binding to and clustering of said target receptor. It is understood that the terms “extracellular vesicle”, “vesicle”, can refer to “engineered extracellular vesicle” based on context. The term “modified” may be used interchangeable with “engineered”, for example a modified extracellular vesicle is an engineered extracellular vesicle, or a modified protein may be an engineered protein.

An engineered extracellular vesicle may be designed to display any fusion polypeptide provided herein. An engineered extracellular vesicle may be designed to display any protein of interest or fragment thereof provided herein. An engineered extracellular vesicle may be designed to comprise any membrane anchoring domain or fragment thereof provided herein, wherein the membrane anchoring domain may be selected from a sequence of any protein of interest provided herein. An engineered extracellular vesicle may be designed to activate, block, agonize, antagonize, or modulate any target protein provided herein. It is understood that the intended function of the engineered extracellular vesicle described herein is conferred by the fusion polypeptide associated with said engineered extracellular vesicle.

An engineered extracellular vesicle may be designed to comprise a Type I transmembrane fusion polypeptide. An engineered extracellular vesicle may be designed to comprise a Type II transmembrane fusion polypeptide. An engineered extracellular vesicle may be designed to comprise a lipid anchored fusion polypeptide. An engineered extracellular vesicle may be designed to comprise a multi-pass membrane protein fusion polypeptide.

Extracellular vesicles may carry cargo, wherein the term “cargo” refers to peptides, proteins, nucleic acids, lipids, metabolites, carbohydrates, biomolecules, small molecules, large molecules, vesicles, organelles, or fragments thereof. In some embodiments, cargo may refer to existing drugs or therapeutics known in the art. Extracellular vesicle cargo may be located within the internal space of the extracellular vesicle. Extracellular vesicle cargo may be membrane bound and span one or both layers of the extracellular vesicle phospholipid bilayer (for example a transmembrane protein). Extracellular vesicle cargo may be in contact with the external or internal surface of the extracellular vesicle, for example through a covalent bond or a non-covalent bond. Extracellular vesicle cargo may be an engineered fusion polypeptide that is bound to the EV membrane. Extracellular vesicle cargo may be an engineered fusion polypeptide that spans one or both layers of the extracellular vesicle phospholipid bilayer (for example a transmembrane fusion polypeptide). Extracellular vesicle cargo may be an engineered fusion polypeptide that is located within the internal space of the EV. Extracellular vesicle cargo may be an engineered fusion polypeptide that is located within the internal space of the EV and is not bound to the internal phospholipid layer of the EV. The phospholipid bilayer of the extracellular vesicle may comprise one or more transmembrane proteins, wherein a portion of the one or more transmembrane membrane proteins is located within the internal space of the extracellular vesicle. The phospholipid bilayer of the extracellular vesicle may comprise one or more transmembrane proteins, wherein the one or more transmembrane membrane proteins comprises a domain (e.g., ectodomain) on the exterior of the extracellular vesicle. The phospholipid bilayer of the extracellular vesicle may comprise one or more transmembrane proteins, wherein the one or more transmembrane membrane proteins comprises a domain on the interior of the extracellular vesicle. Cargo may refer to a protein on the luminal side (e.g., in the internal space) of the extracellular vesicle wherein said protein encodes a vesicle targeting domain that may be in contact with the interior phospholipid layer of the extracellular vesicle. Cargo may refer to a protein on the luminal side (e.g., in the internal space) of the extracellular vesicle wherein said protein encodes a vesicle targeting domain that may be in contact with the interior phospholipid layer of the extracellular vesicle and wherein said protein may be presented into the internal space of the extracellular vesicle.

As used herein, the terms “membrane protein” and “membrane targeting polypeptide” refer to a polypeptide that is directed to a membrane, for example a biological membrane of a cell, wherein the biological membrane of the cell is a plasma or cell membrane. A membrane protein comprises one or more membrane anchoring domain. A membrane protein may be directed to the membrane of a nucleus, mitochondria, endoplasmic reticulum, peroxisome, multi vesicular body. A membrane protein may be attached to or associated with the membrane of a cell, for example a plasma membrane or cell membrane. A membrane protein may be attached to or associated with the membrane of an artificial vesicle, for example a lipid nanoparticle. In some embodiments, a fusion polypeptide presented herein may be attached to or associated with a membrane of an artificial vesicle, for example a lipid nanoparticle. In various aspects, the lipid nanoparticle comprises an ionizable cationic lipid, a neutral lipid, a sterol, and a PEG-modified lipid. In some aspects, the lipid nanoparticle comprises 20-60% ionizable cationic lipid, 5-25% neutral lipid, 25-55% cholesterol, and 0.5-15% PEG-modified lipid. In some aspects, the lipid nanoparticle comprises 50% ionizable cationic lipid, 10% neutral lipid, 38.5% sterol, and 1.5% PEG-modified lipid. In some aspects, neutral lipid is 1,2-distearoyl-sn-glycero-3-phosphocholine (DSPC), the sterol is cholesterol, and the PEG-modified lipid is 1,2-dimyristoyl-racalycero-3-methoxypolyethylene glycol-2000 (PEG-DMG) or PEG-CDMA.

The membrane protein may be an integral membrane protein. The integral membrane protein may be a monotopic, bitopic, or polytopic membrane protein. Polytopic integral membrane proteins may also be referred to as “transmembrane proteins”, wherein the polytopic membrane protein comprises at least one transmembrane amino acid sequence (i.e., a transmembrane domain) spanning the entirety of a membrane, for example a plasma membrane. Bitopic integral membrane proteins refers to polytopic integral membrane proteins with a single transmembrane domain. Monotopic integral membrane proteins are associated with, or attached to, only one side of a membrane and do not span across the entirety of a membrane.

The membrane protein may be a peripheral membrane protein. The peripheral membrane protein may attach to or associate with one or more integral membrane proteins. Peripheral membrane proteins do not span the entirety of a membrane. A peripheral membrane protein penetrates a single lipid layer of a membrane lipid bilayer (i.e., peripheral region of a membrane). Some peripheral membrane proteins may attach to, or associate with, one or more integral membrane proteins and a lipid bilayer simultaneously.

The membrane protein may penetrate a membrane, for example the plasma membrane, spanning the entirety of the plasma membrane. The membrane protein may comprise a transmembrane (TM) amino acid sequence (also referred to as transmembrane domain or transmembrane region) wherein the transmembrane amino acid sequence spans the entirety of a membrane, for example the entirety of the plasma membrane. The membrane protein may comprise a single transmembrane domain or multiple transmembrane domains.

The membrane protein may be a type I membrane protein and can encompass a type I transmembrane protein. The terms “type I membrane protein” and “type I transmembrane protein” refer to a type I membrane protein consisting of a single transmembrane domain, wherein the single transmembrane domain of the type I membrane protein spans the entirety of a membrane, for example a plasma membrane, and the N-terminus of the type I membrane protein is located extracellularly or luminally, and wherein the C-terminus of the type I membrane protein is located in the cytosol.

In some embodiments of any of the aspects, the type I membrane protein is from the group consisting of CD1a, CD1b, CD1c, CD1d, CD1e, LEU1 (CD5), CD6, CD7, CD10, ITGB2 (CD18), CD19, CR2 (CD21), CD27, CD28, CD34, integrin alpha-IIb (ITA2B, CD41), platelet glycoprotein IX (CD42a), platelet glycoprotein Ib alpha chain (CD42b), platelet glycoprotein Ib beta chain (CD42c), platelet glycoprotein V (CD42d), B7-1 (CD80), B7-2 (CD86), OX40 (CD134), glucocorticoid-induced TNFR-related protein (GITR, CD357), inducible T-cell costimulatory (ICOS, CD278), ICOS ligand (ICOSL, CD275), Herpes virus entry mediator A (HVEM, CD270), B7-H3 (CD276), B and T lymphocyte attenuator (BTLA, D272), CTLA-4 (CD152), killer cell immunoglobulin-like receptor family (KIR family, CD158 family: CD158a-k; KIR2DL1, KIR2DL2, KIR2DL3, KIR3 DPI, KIR2DL4, KIR3DL1, KIRDs1, KIR2DL5A, KIR2D15B, KIR2DS5, KIR2DS1, KIR2DS4, KIR2DS2, KIR3DL2), PD-1 (CD279), PD-L1 (CD274), PD-L2 (CD273), T-cell immunoglobulin mucin receptor 1 (TIM-1, CD365), T-cell immunoglobulin mucin receptor 3 (TIM-3, CD366), T-cell immunoglobulin and mucin domain-containing protein 4 (TIM-4), VISTA, sialic acid-binding Ig-like lectin (SIGLEC) 1 (SIGLEC1, CD169), SIGLEC2 (CD22), SIGLEC3 (CD33), SIGLEC5 (CD170), SIGLEC6 (CD328), SIGLEC7 (CD328), SIGLEC8, SIGLEC9 (CD329), SIGLEC10, TIGIT, PVR (CD155), lysosome associated membrane glycoprotein 1 (LAMP1, CD107a), lysosome associated membrane glycoprotein 2 (LAMP2, CD107b), lysosome associated membrane glycoprotein 3 (LAMP3, CD208), PECAM-1 (CD31), STAB-1, NRP2, CEACAM-1 (CD66a), TCR, VTCN1, NCR3LG1, B7-H7 (CD28H), IFNγ receptor 1, IFNγ receptor 2, CD2, CD4, lymphocyte function-associated antigen 3 (LFA-3, CD58), CD8, CD44, CEACAM3 (CD66d), CD96, IGSF2 (CD101), NECTIN1 (HVEC, CD111), NECTIN2 (CD112), NECTIN3 (CD113), DNAX accessory molecule 1 (DNAM-1, CD226), IL2RB (CD122), tyrosine-protein phosphatase no-receptor type substate 1 (SIRPa, CD172a), signal-regulatory protein beta-1 (SIRPB1, CD172b), signal-regulatory protein gamma (SIRPG, CD172g), OX-2 (CD200), OX-2R (CD200R), LAG3 (CD223), LAIR-1 (CD305), NKp30 (CD337), TWEAKR (CD266), CD3d, CD3e, CD3g, ITGAL (CD11a), ITGAM (CD11b), ITGAX (CD11c), ITGAD (CD11d), FCGR3A (CD16a), IL-4 receptor subunit alpha (IL4RA, CD124), IL-2 receptor subunit alpha (IL2RA, CD25), ITGB1 (CD29), CD30, low affinity immunoglobulin gamma Fc region receptor II-a (CD32a), low affinity immunoglobulin gamma Fc region receptor II-b (CD32b), complement receptor type I (CD35), leukosialin (CD43), CD44, receptor-type tyrosine-protein phosphatase C (CD45), membrane cofactor protein (CD46), integrin alpha-1 (CD49a), integrin alpha-2 (CD49b), integrin alpha-3 (CD49c), integrin alpha-4 (CD49d), integrin alpha-5 (CD49e), integrin alpha-6 (CD49f), intercellular adhesion molecule 3 (ICAM-3, CD50), intercellular adhesion molecule 1 (ICAM-1, CD54), ICAM-4 (CD242), integrin alpha V (ITGAV, CD51), integrin beta 3 (ITGB3, CD61), complement decay accelerating factor (CD55), neural adhesion molecule 1 (NCAM-1, CD56), CD62E, CD62L, CD62P, High affinity immunoglobulin gamma Fc receptor I (CD64), macrosialin (CD68), B-cell antigen receptor complex-associated protein alpha chain (CD79a), B-cell antigen receptor complex-associated protein beta chain (CD79b), CD83, leukocyte immunoglobulin-like receptor subfamily A members (CD85G, CD85H, CD85I), leukocyte immunoglobulin-like receptor subfamily B members (CD85A, CD85B, CD85C, CD85D, CD85F, CD85J, CD85K), Immunoglobulin alpha Fc receptor (CD89), CD91, CD93, FAS (CD95), T-cell surface protein tactile (CD96), CD99, semaphoring-D (CD100), immunoglobulin superfamily member 2 (CD101), intercellular adhesion molecule 2 (ICAM-2, CD102), integrin alpha-E (CD103), integrin beta-4 (ITGB4, CD104), endoglin (CD105), vascular cell adhesion protein 1 (VCAM1, CD106), thrombopoietin receptor (CD110), CD114, macrophage colony-stimulating factor 1 receptor (CSF1R, CD115), Granulocyte-macrophage colony-stimulating factor receptor subunit alpha (CSF2RA, CD116), mast/stem cell growth factor receptor Kit (CD117), leukemia inhibitory factor receptor (LIFR, CD118), interferon gamma receptor 1 (CD119), Tumor necrosis factor receptor superfamily member 1A (TNF-R1, CD120a), Tumor necrosis factor receptor superfamily member 1B (TNF-R2, CD120b), Interleukin-1 receptor type 1 (CD121a), Interleukin-1 receptor type 2 (CD121b), Interleukin-2 receptor subunit beta (CD122), Interleukin-3 receptor subunit alpha (IL3RA, CD123), Interleukin-4 receptor subunit alpha (IL4RA, CD124), Interleukin-5 receptor subunit alpha (IL5RA, CD125), Interleukin-6 receptor subunit alpha (IL6RA, CD126), Interleukin-6 receptor subunit beta (IL6ST, CD130), Interleukin-7 receptor subunit alpha (IL7RA, CD127), Interleukin-9 receptor (CD129), Cytokine receptor common subunit beta (CD131), Cytokine receptor common subunit gamma (CD132), CD135, macrophage stimulating protein receptor (CD136), syndecan-1 (CD138), Platelet-derived growth factor receptor alpha (PDGFRA, CD140a), Platelet-derived growth factor receptor beta (PDGFRB, CD140b), thrombomodulin (CD141), CD142, angiotensin converting enzyme (ACE, CD143), cadherin-5 (CD144), melanoma and adhesion molecule (MCAM, CD146), basigin (BSG, CD147), CD148, Signaling lymphocytic activation molecule (SLAM, CD150), SLAM family member 4 (SLAMF4, CD244), signaling lymphocytic activation molecule (SLAM) family member 5 (SLAM5, CD84), SLAM family member 6 (SLAMF6, CD352), SLAM family member 7 (SLAMF7, CD319), SLAM family member 8 (SLAMF8, CD353), SLAM family member 9 (SLAM9), Disintegrin and metalloproteinase domain-containing protein 8 (ADAM8, CD156a), Disintegrin and metalloproteinase domain-containing protein 17 (ADAM17, CD156b), Disintegrin and metalloproteinase domain-containing protein 10 (ADAM10, CD156c), P-selectin glycoprotein 1 (SELPLG, CD162), CD163, CD164, activated leukocyte cell adhesion molecule (ALCAM, CD166), epithelial discoidin domain containing receptor 1 (CD167a), discoidin domain containing receptor 2 (CD167b), neural cell adhesion molecule L1 (L1CAM, CD 171), CD180, endothelial protein C receptor (EPCR, CD201), angiopoietin-1 receptor (CD202b), lymphocyte antigen 75 (CD205), macrophage mannose receptor 1 (CD206), IL-10 receptor subunit alpha (IL10RA, CD210), IL-10 receptor subunit beta (IL10RB, CDw210b), IL-12 receptor subunit beta-1 (IL12RB1, CD212), IL-13 receptor subunit alpha-1 (CD213a1), IL-13 receptor subunit alpha-2 (CD213a2), IL-15 receptor subunit alpha (CD215), IL-17 receptor A (CD217), IL-18 receptor 1 (CD218a), IL-18 receptor accessory protein (CD218b), insulin receptor (CD220), insulin-like growth factor 1 receptor (CD221), cation-independent mannos-6phosphate receptor (CD222), mucin-1 (CD227), T-lymphocyte surface antigen Ly-9 (CD229), plexin-C1 (VESPR, CD232), glycophorin-A (CD235a), glycophorin-B (CD235b), basal cell adhesion molecule (CD239), CD246, T-cell surface glycoprotein CD3 zeta chain (CD247), endosialin (CD248), death receptor 3 (DR3), death receptor 4 (DR4, CD261), death receptor 5 (DR5, CD262), decoy receptor 2 (DcR2, CD264), receptor activator of nuclear factor kappa-B (RANK, CD265), CD271, C-type mannose receptor 2 (CD280), Toll like receptor 1 (CD281), Toll like receptor 2 (CD282), Toll like receptor 3 (CD283), Toll like receptor 4 (CD284), Toll like receptor 6 (CD286), Toll like receptor 8 (CD288), Toll like receptor 9 (CD289), Toll like receptor 10 (CD290), bone morphogenic protein receptor type 1A (CD292), bone morphogenic protein receptor type ID (CwD293), leptin receptor (CD295), CD300a, CD300c, CD302, Neuropilin-1 (CD304), leukocyte-associated immunoglobulin-like receptor 1 (LIAR1, CD305), Fc receptor-like protein 1 (FcRL1, CD307a), Fc receptor-like protein 2 (FcRL2, CD307b), Fc receptor-like protein 3 (FcRL3, CD307c), Fc receptor-like protein 4 (FcRL4, CD307d), Fc receptor-like protein 5 (FcRL5, CD307e), vascular endothelial growth factor receptor 2 (VEGFR2, CD309), prostaglandin F2 receptor negative regulator (PTGFRN, CD315), immunoglobulin superfamily member 8 (IGSF8, CD316), CD320, platelet F11 receptor (F11R, CD321), junctional adhesion molecule B (JAM-B, CD322), cadherin-1 (CD324), cadherin-2 (CD325), epithelial cell adhesion molecule (CD326), fibroblast growth factor 1 (FGFR1, CD331), fibroblast growth factor 2 (FGFR2, CD332), fibroblast growth factor 3 (FGFR3, CD333), fibroblast growth factor 4 (FGFR4, CD334), natural cytotoxicity triggering receptor 1 (NCR1, CD335), natural cytotoxicity triggering receptor 2 (NCR2, CD336), natural cytotoxicity triggering receptor 3 (NCR3, CD337), triggering receptor expressing on myeloid cells 1 (TREM1, CD354), cytotoxic and regulatory T-cell molecule (CRTAM, CD355), tumor necrosis factor receptor superfamily member 21 (CD358), interleukin-21 receptor (IL21R, CD360), protein EVI2B (CD361), syndecan-2 (CD362), V-set and immunoglobulin domain-containing protein 1 (VSIG1), V-set and immunoglobulin domain-containing protein 8 (VSIG8), V-set and immunoglobulin domain-containing protein 3 (VSIG3), V-set and immunoglobulin domain-containing protein 3 (VSIG4), butyrophilin subfamily 3 member A1 (BTN3A1, CD277), butyrophilin subfamily 3 member A2 (BTN3A2), butyrophilin subfamily 2 member A1 (BTN2A1), butyrophilin like protein 8 (BTNL8), butyrophilin subfamily 1 member A1 (BTN1A1), isoforms thereof, fragments thereof, and combinations thereof.

In some embodiments a fusion polypeptide as presented herein may be designed or constructed to be a type I membrane protein, and herein referred to as a type I membrane fusion polypeptide or type I fusion polypeptide.

The membrane protein may be a type II membrane protein. The terms “type II membrane protein” can encompass “type II transmembrane protein” and refer to a type II membrane protein consisting of a single transmembrane domain, wherein the single transmembrane domain of the type II membrane targeting polypeptide spans the entirety of a membrane, for example a plasma membrane, and the C-terminus of the type II membrane protein is located extracellularly or luminally, and wherein the N-terminus of the type II membrane protein is located in the cytosol.

In some embodiments of any of the aspects, the type II membrane protein is from the group consisting of CD27L (CD70), CD40, CD40L (CD154), 4-1BB (CD137), 4-1BBL (CD137L), OX40L (CD252), glucocorticoid-induced TNF-related ligand (GITRL), LIGHT (CD258), TNF-related apoptosis inducing factor (TRAIL, CD253), CLEC7A (CD369), CD30L (CD153), TL1 (TNFSF15), FasL (CD178), NKG2 family ligands (NKG2A, B, C, D, E, F and H), B cell activating factor (BAFF, CD257), TNF-related weak inducer of apoptosis (TWEAK), RBAT (SLC3A1), ATPIB2, CD94, neprilysin (CD10), CD13, BLAST-2 (CD23), Dipeptidyl peptidase 4 (DPP4, ADCP2, CD26), CD38, CLEC2C (CD69), Transferrin receptor protein 1 (CD71), B-cell differentiation antigen Lyb-2 (CD72), HLA class II histocompatibility antigen gamma chain (CD74), CD75, CD77, natural killer cell antigen KLRD1 (CD94), NKG2-A/B-activating NK receptor (CD159a), NKG2-C(CD159c), killer cell lectin-like receptor subfamily B member 1 (CD161), galactoside alpha-(1,2)-fucosyltransferase 1 (FUT1, CD174), 3-galactosyl-N-acetylglucosamide 4-alpha-L-fucosyltransferase (FUT3), ectonucleotide pyrophosphatase/phosphodiesterase (ENPP) family member 1 (ENPP1, CD203a), ENPP family member 3 (ENPP3, CD203c), macrophage scavenger receptor types I and II (CD204), C-type lectin domain family 4 member K (langerin, CD207), dendritic cell-specific ICAM-3-grabbing non-integrin 1 (DC-SIGN, CD209), CD224, CD238, glutamyl aminopeptidase (CD249), receptor activator of nuclear factor kappa-B ligand (RANKL, CD254), CD298, DC-SIGN related protein (DC-SIGNR, CD299), C-type lectin domain family 10 member A (CLEC10A, CD301), C-type lectin domain family 4 member C (CLEC4C, CD303), NKG2-D type II integral membrane protein (KLRK1, CD314), bone marrow stromal antigen 2 (BST2, CD317), transmembrane and associated with src kinases (TRASK, CD318), protein jagged-1 (CD339), human epidermal growth factor 2 (HER2, CD340), C-type lectin domain family 4 member A (CLEC4A, CD367), C-type lectin domain family 4 member D (CLEC4D, CD368), C-type lectin domain family 7 member A (CLEC7A, CD369), C-type lectin domain family 9 member A (CLEC9A, CD370), C-type lectin domain family 12 member A (CLEC12A, CD371), SLC3A2 (CD98 heavy chain, 4F2), tumor necrosis factor (TNF-alpha), lymphotoxin-alpha (LTA, LT-α) also known as tumor necrosis factor ligand superfamily member 1 (TNF-beta, TNF-β), tumor necrosis family ligand superfamily member 3 also known as lymphotoxin beta (LTB, TNF-C), tumor necrosis factor ligand superfamily 15 (TL1A), isoforms thereof, fragments thereof, and combinations thereof.

The membrane protein may be a type III membrane protein, also referred to as a type III transmembrane protein. The terms “type III membrane protein” refers to a type III membrane protein comprises a single transmembrane domain, wherein the transmembrane domain of the type III membrane protein spans the entirety of a membrane, for example a plasma membrane, and the N-terminus of the type III membrane protein is located extracellularly or luminally, and wherein the C-terminus of the type III membrane protein is located in the cytosol.

In some embodiments a fusion polypeptide as presented herein may be designed or constructed to be a type II membrane protein, and herein referred to as a type II membrane fusion polypeptide or type II fusion polypeptide.

In some embodiments of any of the aspects, the type III membrane protein is from the group consisting of B cell activating factor (BAFFR, CD268), glycophorin-C(CD236), transmembrane activator and CAML interactor (TACI, CD267), B-cell maturation protein (BCM, CD269), isoforms thereof, fragments thereof, and combinations thereof.

The membrane protein may be a multi-pass membrane protein, also referred to as a multi-pass transmembrane protein. The terms “multi-pass membrane protein” refers to a membrane protein comprising multiple transmembrane domains. In some multi-pass membrane proteins, the transmembrane domains of the multi-pass membrane protein span the entirety of the plasma membrane, and the N-terminus of the multi-pass membrane protein is located extracellularly or luminally, and wherein the C-terminus of the multi-pass membrane protein is located in the cytosol. In some multi-pass membrane proteins, the transmembrane domains of the multi-pass membrane protein span the entirety of the plasma membrane, and the C-terminus of the multi-pass membrane protein is located extracellularly or luminally, and wherein the N-terminus of the multi-pass membrane protein is located in the cytosol. In some multi-pass membrane proteins, the transmembrane domains of the multi-pass membrane protein span the entirety of the plasma membrane, and the C-terminus of the multi-pass membrane protein is located extracellularly or luminally, and wherein the N-terminus of the multi-pass membrane protein is located extracellularly or luminally. In some multi-pass membrane proteins, the transmembrane domains of the multi-pass membrane protein span the entirety of the plasma membrane, and the C-terminus of the multi-pass membrane protein is located in the cytosol, and wherein the N-terminus of the multi-pass membrane protein is located in the cytosol.

In some embodiments of any of the aspects, the multi-pass membrane protein is from the group consisting of Alpha-2A adrenergic receptor (A2AR, ADRA2A), adenosine receptor A2b (A2BR, ADORA2B), NOX2, SLC7A5 (CD98 light chain), CD39, CD47, PVRIG (CD112R), CD9, CD20, CD36, CD37, CD53, CD63, CD81, CD82, C5a receptor (CD88), CD92, CD97, prominin-1 (CD133), CD151, high affinity interleukin-8 receptor A (IL8RA, CXCR1, CD181), high affinity interleukin-8 receptor B (IL8RB, CXCR2, CD182), C—X—C chemokine receptor (CXCR) type 3 (CXCR3, CD183), CXCR4 (CD184), CXCR5 (CD185), CXCR6 (CD186), C—C chemokine (CCR) type 1 (CCR1, CD191), CCR2 (CD192), CCR3 (CD193), CCR4 (CD194), CCR5 (CD195), CCR6 (CD196), CCR7 (CD197), CCR8 (CDw189), CCR9 (CDw199), CD231, solute carrier family 4 member 1 (SLC4A1, CD233), Duffy antigen/chemokine receptor (DARC, CD234), blood group Rh (CE) polypeptide (CD240CE), blood group Rh (D) polypeptide (CD240D), ammonium transporter Rh type A (CD241), CD243, calcium signal-modulating cyclophilin ligand (CAMLG), prostaglandin D2 receptor 2 (PTGDR2, CD294), EGF-like module receptor 2 (CD312), CD338, frizzled-4 (CD344), frizzled-9 (CD349), frizzled-10 (CD350), sphingosine 1-phosphate receptor 1 (CD363), isoforms thereof, fragments thereof, and combinations thereof.

In some embodiments of any of the aspects, the multi-pass membrane protein is a tetraspanin from the group consisting of tetraspanin TSPAN1 (TSP-1), TSPAN2 (TSP-2), TSPAN3 (TSP-3), TSPAN4 (TSP-4, NAG-2), TSPAN5 (TSP-5), TSPAN6 (TSP-6), TSPAN7 (CD231, TALLA-1, A15), TSPAN8 (CO-029), TSPAN9 (NET-5), TSPAN10 (OCULOSPANIN), TSPAN11 (CD151-like), TSPAN12 (NET-2), TSPAN13 (NET-6), TSPAN14, TSPAN15 (NET-7), TSPAN16 (TM4-B), TSPAN17, TSPAN18, TSPAN19, TSPAN20 (UP1b, UPK1B), TSPAN21 (UP1a, UPK1A), TSPAN22 (RDS, PRPH2), TSPAN23 (ROM1), TSPAN24 (CD151), TSPAN25 (CD53), TSPAN26 (CD37), TSPAN27 (CD82), TSPAN28 (CD81), TSPAN29 (CD9), TSPAN30 (CD63), TSPAN31 (SAS), TSPAN32 (TSSC6), TSPAN33, isoforms thereof, fragments thereof, and combinations thereof.

The membrane protein may be a lipid-anchored membrane protein. Lipid anchored membrane proteins may be covalently attached to fatty acid acyl chains on the cytoplasmic side of a membrane, for example a plasma membrane. Lipid-anchored membrane proteins may be attached to, or associate with, a membrane via fatty acyl chains, wherein the fatty acyl chains are selected from the group consisting of palmitic acid and myristic acid. Lipid-anchored membrane proteins may be attached to, or associate with, a membrane via farnesyl or geranylgeranyl isoprenoids. Lipid-anchored membrane proteins may be attached to, or associate with, a membrane, for example a cell plasma membrane, via palmitoylation, myristoylation, or prenylation. Lipid-anchored membrane proteins may be attached to, or associate with, a membrane, for example a cell plasma membrane, via cholesteroylation, wherein a cholesterol molecule is esterified to a C-terminal glycine residue. Lipid-anchored membrane proteins may be attached to, or associate with, a membrane, for example a cell plasma membrane, via glypiation, wherein a glycosylphosphatidylinositol (GPI) anchor is added to the C-terminus of a protein by a transaminidase in the lumen of the endoplasmic reticulum. Membrane proteins may be attached to, or associate with, a membrane, for example a cell plasma membrane, via a lipid anchor at the N-terminus and a polybasic domain. An example of a membrane protein attached to a cell membrane via a lipid anchor at the N-terminus and a polybasic domain is the MARCKS protein.

In some embodiments of any of the aspects, the lipid anchored membrane protein is from the group consisting of CD160, RGMB, CEACAM8 (CD66b, CD67), CEACAM6 (CD66c), CEACAM5 (CD66e), CD73, CD14, FCGR3B (CD16b), CD24, BLAST-1 (CD48), CAMPATH-1 (CD52), CD59, CD87, CD90, semaphorin-7A (CD108), CD109, bone marrow stromal cell antigen 1 (BSTI, CD157), CD177, melanotransferrin (CD228), CD230, decoy receptor 1 (DcR1, CD263), CD296, CD297, isoforms thereof, fragments thereof, and combinations thereof.

A membrane anchoring domain may be a transmembrane domain from a type I membrane protein. A membrane anchoring domain may be a transmembrane domain from a type II membrane protein. A membrane anchoring domain may be a transmembrane domain from a type III membrane protein. A membrane anchoring domain may be a transmembrane domain from a multi-pass membrane protein. A membrane anchoring domain may be a cholesteroylation, prenylation, glypiation, or fatty acylation domain from a lipid-anchored membrane protein. A membrane anchoring domain may be a cholesteroylation, prenylation, glypiation, or fatty acylation moiety from a lipid-anchored membrane protein. A membrane anchoring domain may be a lipid anchor at the N-terminus and a polybasic domain from MARCKS or a fragment thereof.

As used herein, membrane anchoring domains (e.g., transmembrane domains) can be structurally identified using methods known to those of skill in the art, such as sequence analysis programs that identify hydrophobic and hydrophilic domains, for example using the sequence analysis program TMHMM Server, v. 2.0-DTU, Erik L. L. Sonnhammer, Gunnar von Heijne, and Anders Krogh: A hidden Markov model for predicting transmembrane helices in protein sequences. In Proc. of Sixth Int. Conf. on Intelligent Systems for Molecular Biology, p 175-182 Ed J. Glasgow, T. Littlejohn, F. Major, R. Lathrop, D. Sankoff, and C. Sensen Menlo Park, CA: AAAI Press, 1998, which is incorporated herein by reference in its entirety. A membrane anchoring domain from a POI as identified using the cited Glasgow et. al. reference may be engineered into a fusion polypeptide as a vesicle targeting domain. It is understood that there are various other sequence analysis programs available to one of skill in the art that can be used to predict membrane anchoring domains. For example, the sequence analysis program Deep TMHMM may be used to predict the topology of transmembrane proteins (Jeppe Hallgren, Konstantinos D. Tsirigos, Mads D. Pedersen, José Juan Almagro Armenteros, Paolo Marcatili, Henrik Nielsen, Anders Krogh and Ole Winther (2022). Deep TMHMM predicts alpha and beta transmembrane proteins using deep neural networks. doi.org/10.1101/2022.04.08.487609, which is incorporated herein by reference in its entirety). Membrane anchoring domains may also be identified using the UniProt database. Other membrane anchor prediction analysis programs are HMMTOP (G. E Tusnády and I. Simon (1998) Principles Governing Amino Acid Composition of Integral Membrane Proteins: Applications to Topology Prediction.” J. Mol. Biol. 283, 489-506., and “G. E Tusnády and I. Simon (2001) The HMMTOP transmembrane topology prediction server” Bioinformatics 17, 849-850.), MEMSAT/MEMSAT3/PSIPRED (Jones, D. T., Taylor, W. R., and Thornton, J. M. (1994) Biochemistry, 33:3038-3049., and Buchan D W A, Jones D T (2019). The PSIPRED Protein Analysis Workbench: 20 years on. Nucleic Acids Research. doi.org/10.1093/nar/gkz297), PRED-TMR2 (Pasquier C, Hamodrakas S J. A hierarchical artificial neural network system for the classification of transmembrane proteins. Protein Eng. 1999 August; 12 (8): 631-4. doi: 10.1093/protein/12.8.631. PMID: 10469822), and SPLIT 4.0 (Juretic, D., Jeroncic, A. and Zucic, D. “Sequence analysis of membrane proteins with the web server SPLIT.” Croatica Chemica Acta Vol. 72, No. 4, pp. 975-997, 1999). It is understood that a membrane anchoring domain from a POI, wherein said membrane anchoring domain is identified using a membrane prediction program may be engineered into a fusion polypeptide as a vesicle targeting domain. All references cited herein are incorporated by reference in their entirety as though fully set forth.

In various embodiments a fragment of a membrane anchoring domain can be at least 75%, 80%, 85%, 90%, or 95% of a membrane anchoring domain.

A vesicle targeting domain may include, but is not limited to, one or more prenylation site, fatty acylation site, and/or glycosylphosphatidylinositol (GPI) linked protein. One preferred embodiment of a vesicle targeting domain is the GPI sequence from CD55. Another preferred embodiment of a vesicle targeting domain is the GPI sequence from CD59. Another embodiment of a vesicle targeting domain is the C1C2 domain from MFGE8. Other embodiments of sequences for vesicle targeting domains include transmembrane regions of CD9 (for example transmembrane 2 or 3 of CD9, CD9tm2 or CD9tm3, respectively), K-Ras (for example K-Ras4A and K-Ras4B), transmembrane domain from A Disintegrin and Metalloproteinase Domain-containing protein 10 (ADAM10, also known as CDw156 or CD156c) or other ADAM proteins. Vesicle targeting domains may include one or more sequences from 4F2 (for example 4F2 encoded by the solute carrier family 3 member 2 (SLC3A2) gene which makes up the heavy subunit of CD98). Vesicle targeting domains can include a sequence for one or more myristoylation sites. For example, the protein sequence for a myristoylation site from myristoylated alanine-rich C-kinase substrate (MARCKS) protein. Vesicle targeting domains can include a sequence for one or more palmitoylation sites. For example, the myristoylation sequence from the MARCKS protein may be modified to encode for a palmitoylation site. All isoforms, or fragments or the like known by one of ordinary skill in the art are encompassed by the present invention.

Vesicle targeting domains may include transmembrane sequences from Homo sapiens transferrin receptor 2 (TFR2), transcript variant 1 (transferrin receptor protein 2 isoform 1) or versions thereof. In a preferred embodiment, the vesicle targeting domain may be a transmembrane domain from CD298.

As used herein, the terms “proteins” and “peptides” and “polypeptides” designate a series of amino acid residues connected to the other by peptide bonds between the alpha-amino and carboxy groups of adjacent residues. Although “protein” is often used in reference to relatively large polypeptides, and “peptide” is often used in reference to small polypeptides, usage of these terms in the art overlaps and varies. The term “peptide” as used herein refers to peptides, polypeptides, proteins and fragments of proteins, unless otherwise noted. The term “polypeptide” as used herein refers to peptides, polypeptides, proteins and fragments of proteins, unless otherwise noted. The terms “protein” and “peptide” are used interchangeably herein when referring to a gene product (i.e., a translated RNA sequence) and fragments thereof. Thus, exemplary peptides or proteins include gene products, naturally occurring proteins, homologs, orthologs, paralogs, fragments and other equivalents, variants, fragments, and analogs of the foregoing.

As used herein, the terms “N-terminus” and “N-terminal” and “N-term” and “amino terminus” and “amine terminus” and “NH2” are used interchangeably herein to designate the free unattached amine group (—NH₂) located on the alpha carbo of the first amino acid a polypeptide or protein. The N-terminal designates the front or beginning end (with respect to translation) of a protein or polypeptide sequence. The free amine group is not linked to another amino acid by a peptide bond in the polypeptide. The N-terminus is the start of a protein or polypeptide. All protein and polypeptide sequences herein are written N-terminus to C-terminus, left to right.

As used herein, the terms “C-terminus” and “C-terminal” and “C-term” and “carboxyl-terminus” and “carboxy-terminus” and “carboxyl-terminal” and “COOH” are used interchangeably herein to designate the free unattached carboxylic acid (carboxyl) group (—COOH) located on the alpha carbon of the last amino acid a polypeptide or protein. The C-terminal designates the back end or ending (with respect to translation) of a protein or polypeptide sequence. The free carboxylic acid group is not linked to another amino acid by a peptide bond in the polypeptide. The C-terminus is the end of a protein or polypeptide. All protein and polypeptide sequences herein are written N-terminus to C-terminus, left to right.

As used herein, the terms “linker” and “linker polypeptide” and “polypeptide linker” in the context of peptides, polypeptides and proteins refer to a protein sequence of amino acids that is used to connect two polypeptides. The term linker in the context of nucleic acids refer to the nucleic acid sequence which encodes for a linker polypeptide. The linker may be flexible, rigid, or cleavable. Further, the linker can be linked directly or via another linker (e.g., a peptide of one, two, three, four, five, six, seven, eight, nine, ten or more amino acids) to the fusion polypeptides described herein. Linkers can be configured according to a specific need, e.g., based on at least one of the following characteristics. In some embodiments of any of the aspects, linkers can be configured to have a sufficient length and flexibility such that it can allow for a proteolysis at a target cleavage site, for example proteolytic cleavage at a proteolytic site. In some embodiments of any of the aspects, linkers can be configured to allow multimerization of the fusion polypeptides provided herein, for example two fusion polypeptides multimerizing to form a dimer. In some embodiments of any of the aspects, linkers can be configured to allow multimerization of one or more fusion polypeptides provided herein, for example two or more fusion polypeptides multimerize to form a multimer, wherein the multimerization of the two or more fusion polypeptides is mediated by covalent or noncovalent intermolecular forces between linkers of the said two or more fusion polypeptides. In some embodiments of any of the aspects, linkers can be configured to facilitate expression and purification of the fusion polypeptides or engineered extracellular vesicles provided herein. In some embodiments of any of the aspects, a linker can be configured to have any length in a form of a peptide, peptidomimetic, an aptamer, a protein, a nucleic acid (e.g., DNA or RNA), or any combinations thereof.

As used herein, the terms “fusion protein” and “fusion polypeptide” refer to a single chimeric protein comprising a protein of interest (e.g. checkpoint protein) joined to another protein or protein fragment (e.g. at least one membrane anchoring domain from at least one membrane protein), wherein the components of the fusion protein are linked to each other by peptide-bonds, either directly or through a linker. The “fusion protein” and “fusion polypeptide” may comprise a signaling domain wherein the signaling domain is a fragment or an active fragment from a protein of interest. The terms “fusion protein” or “fusion polypeptide” may comprise a multi-effector domain wherein the multi-effector domain further comprises active fragments of a protein of interest, for example TNF homology domains connected via linkers. The fusion protein or fusion polypeptide may comprise a vesicle targeting domain, wherein the vesicle targeting domain comprises a sequence from a membrane protein (e.g., a membrane anchoring domain). In some embodiments, the multi-effector domain and vesicle targeting domain of the fusion polypeptide are connected via linkers.

The membrane anchoring domain or vesicle targeting domain of the fusion protein may enhance incorporation of the fusion protein onto and/or into the membrane of a vesicle, for example the internal and/or external leaflet of the phospholipid bilayer of an exosome membrane. The fusion protein may have at least a part of an amino acid sequence of an immune checkpoint protein or proteins involved in immune synapses. The fusion protein may have at least a part of an amino acid sequence of A2AR, VTCN1, Galectin 9, FGL-1, PECAM-1, TSG-6, STAB-1, NRP1, NRP2, SEMA3A, SEMA3F, RGMB, TIM-3, TIM-4, TIGIT, HLA class I, HLA class II, VISTA, HMGB1, phosphatidylserine, T-cell receptor (TCR), SHP-1, SHP-2, FBXO38, SH2D1A, B7RP1, IDO, NOX2, TNFRSF18, B7-H4, B7-H5, SISP1, B7-H6, B7-H7, APLNR, IFN Y, PD-1, WNT5A, IL-6, IL-10, NKG2 family of C-type lectin receptors, ligands of NKG2 family, killer cell immunoglobulin-like receptors, CD2, CD4, CD8, CD27, CD27 ligand (CD70), CD28, CD28H, CD39, CD40, CD44, CD47, CD63, CD66a, CD80, B7-2, CD86, CD73, CD94, CD96, CD101, CD112, CD112R, CD122, CD134, CD137 (4-1BB), CD137 ligand (4-1BBL), CD152, CD154, CD155, CD158, CD158a, CD158g, CD158h, KIR2DL1, KIR2DS1, KIRDS3, KIR2DS5, CD160, CD172a, CD200, CD200R, CD223, CD226, CD252, CD270, CD272, CD273, CD274, CD275, CD276, CD278, CD279 (PD-1), CD279 ligand (PD-L1/PDL-2), CD328, CD329, and/or CD337. The fusion protein may have a polypeptide linker sequence (e.g., an Fc domain and/or a GSSG (SEQ ID NO: 154) linker), followed by an amino acid sequence coding for an anchor protein sequence (e.g., a prenylation site, fatty acylation site, or a GPI sequence) or any isoform thereof, fragment thereof, or a ligand to the aforementioned proteins thereof, or the like known by one of ordinary skill in the art.

As used herein, the term “immune synapse” and “cell synapse” refer to cell-to-cell interaction wherein said interaction results in activation, suppression, and/or adhesion of either one or more cells. Immune synapse or cell synapse are mediated by proteins that may be cytoplasmic, membrane bound, membrane associated, and/or secreted. Immune or cell synapses may be mediated by one or more “immune checkpoint proteins” which herein refers to any protein that is involved in maintaining immune homeostasis or plays a role in regulating immune activation or suppression. Immune checkpoint proteins may be cytoplasmic, membrane bound, membrane associated, and/or secreted.

As used herein, the term “fragment” or “active fragment” or “active domain” in the context of nucleic acids refer to a portion of a nucleic acid provided herein that retains the ability to be expressed or translated by a cell.

As used herein, the term “fragment” in the context of peptides, polypeptides and proteins refer to a portion of a peptide, polypeptide or protein provided herein. In various embodiments, the fragment can be an active fragment.

The terms “active fragment” or “active domain” in the context of peptides, polypeptides and proteins refer to a portion of a peptide, polypeptide (including fusion polypeptide) or protein (including fusion protein) provided herein that retains binding ability to a target protein. For example, the ““active fragment” or “active domain” retains at least 50%, 60%, 70%, 80%, 90%, 95%, 96%, 97%, 98%, or 99% of the binding affinity to a target protein as compared to its full-length peptide, polypeptide or protein. Binding affinity can be determined by one of ordinary skill in the art using various assays available.

In various embodiments, “active fragment” or “active domain” includes a portion of a peptide, polypeptide (including fusion polypeptide) or protein (including fusion protein) provided herein that retains binding ability to a target protein and activating, blocking, agonizing, antagonizing, stimulating, repressing, or modulating said target protein. For example, the “active fragment” or “active domain” retains at least 50%, 60%, 70%, 80%, 90%, 95%, 96%, 97%, 98%, or 99% of the binding affinity and at least 50%, 60%, 70%, 80%, 90%, 95%, 96%, 97%, 98%, or 99% ability for activating, blocking, agonizing, antagonizing, stimulating, repressing, or modulating said target protein to a target protein as compared to its full length peptide, polypeptide or protein. Binding affinity, and ability for activating, blocking, agonizing, antagonizing, stimulating, repressing, or modulating said target protein can be determined by one of ordinary skill in the art using various assays available.

In various embodiments terms “active fragment” or “active domain” includes a portion of a peptide, polypeptide (including fusion polypeptide) or protein (including fusion protein) provided herein that retains the function to agonize a target receptor or target protein. For example, the “active fragment” or “active domain” retains at least 50%, 60%, 70%, 80%, 90%, 95%, 96%, 97%, 98%, or 99% of its function to agonize a target receptor or target protein compared to its full-length peptide, polypeptide or protein. Agonizing ability can be determined by one of ordinary skill in the art using various assays available.

In various embodiments terms “active fragment” or “active domain” includes a portion of a peptide, polypeptide (including fusion polypeptide) or protein (including fusion protein) provided herein that retains the function to antagonize a target receptor or target protein. For example, the “active fragment” or “active domain” retains at least 50%, 60%, 70%, 80%, 90%, 95%, 96%, 97%, 98%, or 99% of its function to antagonize a target receptor or target protein compared to its full-length peptide, polypeptide or protein. Antagonizing ability can be determined by one of ordinary skill in the art using various assays available.

In various embodiments, “active fragment” or “active domain” in the context of peptides, polypeptides and proteins of the signaling domain include a portion of a peptide, polypeptide (including fusion polypeptide) or proteins (including fusion protein) provided herein that comprises the extracellular domain of a transmembrane protein.

In various embodiments, “active fragment” or “active domain” in the context of peptides, polypeptides and proteins of the signaling domain include a portion of a peptide, polypeptide (including fusion polypeptide) or protein (including fusion protein) provided herein that comprises a TNF homology domain.

In various embodiments, “active fragment” or “active domain” in the context of peptides, polypeptides and proteins include a portion of a peptide, polypeptide (including fusion polypeptide) or protein (including fusion protein) provided herein that is at least 50% of the peptide, polypeptide, or protein. In some embodiments, the “active fragment” or “active domain” comprises at least 55%, 60%, 70%, 75%, 80%, 85%, 90%, or 95% of the peptide, polypeptide, or protein.

In one embodiment, the active fragment of 4-1BBL (nucleic acid sequence SEQ ID NO: 105 and amino acid SEQ ID NO: 106) may be between amino acid sequences 71-254. In another embodiment, the active fragment of GITRL (nucleic acid sequence SEQ ID NO: 107 and amino acid SEQ ID NO: 108) may be between amino acid sequences 56-177. In another embodiment, the active fragment of OX40L (nucleic acid sequence SEQ ID NO: 109 and amino acid SEQ ID NO: 110) may be between amino acid sequences 54-183. In another embodiment, the active fragment of CD27L (nucleic acid sequence SEQ ID NO: 111 and amino acid SEQ ID NO: 112) may be between amino acid sequences 53-193. In another embodiment, the active fragment of CD30L (nucleic acid sequence SEQ ID NO: 113 and amino acid SEQ ID NO: 114) may be between amino acid sequences 99-234. In another embodiment, the active fragment of CD40L (nucleic acid sequence SEQ ID NO: 115 and amino acid SEQ ID NO: 116) may be between amino acid sequences 117-261. In another embodiment, the active fragment of LIGHT (nucleic acid sequence SEQ ID NO: 117 and amino acid SEQ ID NO: 118) may be between amino acid sequences 92-240. In another embodiment, the active fragment of TRAIL (nucleic acid sequence SEQ ID NO: 119 and amino acid SEQ ID NO: 120) may be between amino acid sequences 120-281. In another embodiment, the active fragment of RANKL (nucleic acid sequence SEQ ID NO: 121 and amino acid SEQ ID NO: 122) may be between amino acid sequences 161-317. In another embodiment, the active fragment of TL1A (nucleic acid sequence SEQ ID NO: 123 and amino acid SEQ ID NO: 124) may be between amino acid sequences 93-251. In another embodiment, the active fragment of FASL (nucleic acid sequence SEQ ID NO: 125 and amino acid SEQ ID NO: 126) may be between amino acid sequences 144-281. In another embodiment, the active fragment of BAFF (nucleic acid sequence SEQ ID NO: 127 and amino acid SEQ ID NO: 128) may be between amino acid sequences 140-285. In another embodiment, the active fragment of APRIL (nucleic acid sequence SEQ ID NO: 129 and amino acid SEQ ID NO: 130) may be between amino acid sequences 111-250. In another embodiment, the active fragment of TWEAK (nucleic acid sequence SEQ ID NO: 131 and amino acid SEQ ID NO: 132) may be between amino acid sequences 95-249. In another embodiment, the active fragment of TNF (nucleic acid sequence SEQ ID NO: 133 and amino acid SEQ ID NO: 134) may be between amino acid sequences 78-233. In another embodiment, the active fragment of LTA (nucleic acid sequence SEQ ID NO: 135 and amino acid SEQ ID NO: 136) may be between amino acid sequences 35-205. In another embodiment, the active fragment is a fragment of LTB (nucleic acid sequence SEQ ID NO: 137 and amino acid SEQ ID NO: 138). In another embodiment, the active fragment is a fragment of ectodysplasin (nucleic acid sequence SEQ ID NO: 139 and amino acid SEQ ID NO: 140).

In various embodiments, “active fragment” includes a region of a polypeptide or fusion polypeptide provided herein that retains or mostly retains its natural three-dimensional protein structure. In various embodiments, “active fragment” includes a region of a polypeptide or fusion polypeptide provided herein that retains a desired function. In various embodiments, “active fragment” includes a region of a polypeptide or fusion polypeptide provided herein that retains binding specificity to a target protein. For example, an active fragment of a TNFSF protein refers to the fragment that retains binding specificity to a target protein (e.g., TNFSF receptor) and retains the ability to activate/agonize said target protein. For example, an active fragment of a TNFSF protein refers to the fragment that retains binding specificity to a target protein (e.g., TNFSF receptor) and retains the ability to agonize said target protein. For example, an active fragment of 4-1BBL may retain the ability to agonize the target protein 4-1BB receptor. For example, an active fragment of a TNFSF protein refers to the fragment that retains binding specificity to a target protein (e.g., TNFSF receptor) and retains the ability to inactivate said target protein. For example, an active fragment of a TNFSF protein refers to the fragment that retains binding specificity to a target protein (e.g., TNFSF receptor) and retains the ability to antagonize said target protein. An active fragment of a fusion polypeptide may also refer to the multi-effector domain. In some embodiments, the active fragment retains the ability to activate a target protein, thereby increasing the activity of said target protein (e.g., suppressing or inducing an immune response). In some embodiments, the active fragment retains the ability to activate a target protein, thereby decreasing the activity of said target protein (e.g., suppressing or inducing an immune response).

As used herein, the terms “domain” and “protein domain” refer to a region of a protein that is self-stabilizing and that has a distinct function and/or structural units in a protein. For example, a “signaling domain” may refer to a region of a polypeptide that has the function of inducing a signal. For example, the ectodomain of 4-1BBL comprises a signaling domain, wherein the 4-1BBL signaling domain binds to and agonizes its cognate target receptor 4-1BB. A domain as used herein may also refer to a region of a protein's polypeptide sequence. In various embodiments, “domain” or “protein domain” include a general region of a protein's polypeptide sequence. It should be understood by one of ordinary skill in the art that domain may be a fragment, active fragment, or multi-effector domain of a polypeptide.

As used herein, the term “lipid anchoring domain” refers to a region of a protein (e.g., a lipid anchored membrane protein) bound to the lipid bilayer of a membrane. In general, a lipid anchoring domain is bound to the lipid bilayer through a posttranslational modification by the attachment of at least one lipid or fatty acid, e.g., farnesyl, palmitate and myristate.

As used herein, the terms “multi-effector domain” and “multi-effector signaling domain” refer to a single continuous polypeptide comprising multiple protein of interest domains or fragments thereof, wherein the multiple protein of interest domains or fragments thereof are connected to one another via covalent peptide bonds to form a single chain polypeptide. The single chain polypeptide multi-effector domain may comprise 1, 2, 3, 4, 5, 6, 7, 8, 9, 10, or more domains from proteins of interest or fragments thereof. For example, a multi-effector domain may be a recombinant polypeptide comprising of a first amino acid sequence from a protein of interest, a second amino acid sequence from a protein of interest, and a third amino acid sequence from a protein of interest, wherein the first and second amino acid sequence are covalently linked via a peptide bond, wherein the peptide bond links the carboxyl terminus of the first amino acid sequence to the amino terminus of the second amino acid sequence, and wherein the second amino acid sequence and third amino acid sequence are covalently linked via peptide bond, wherein the peptide bond links the carboxyl terminus of the second amino acid sequence to the amino terminus of the third amino acid sequence. The single chain polypeptide multi-effector domain may comprise one or more linkers. The multi-effector domain may be covalently linked via peptide bonds to one or more linkers. The single chain polypeptide multi-effector domain may be covalently linked via peptide bond to one or more vesicle targeting domains. The single chain polypeptide multi-effector domain may comprise one or more protein of interest. For example, the single chain polypeptide multi-effector domain may comprise three or more protein of interest. In one embodiment the single chain polypeptide multi-effector domain comprises three proteins of interest (POI) or a fragment thereof, wherein the proteins of interest or fragments thereof are linked covalently via a peptide bond in a single chain polypeptide. In one embodiment the single chain polypeptide multi-effector domain comprises three proteins of interest or fragments thereof, wherein the protein of interest or fragments thereof are linked covalently via a peptide bond in a single polypeptide, and wherein a linker is between each protein of interest or fragment thereof. For example, a single chain polypeptide muti-effector domain may comprise the following domain organization: N-terminus-linker-POI domain-linker-POI-linker-POI-C-terminus. The N-terminus of the single chain polypeptide muti-effector domain may be covalently linked via a peptide bond to a linker polypeptide (for example an Fc or Fc mutein linker) or to a vesicle targeting domain (e.g., a vesicle targeting domain comprising one or more membrane anchoring domains from a POI). The single chain polypeptide muti-effector domain may comprise more than three POI domains. In some embodiments neighboring POI domains are not separated by a linker. In other embodiments neighboring POI domains may be separated by multiple linkers. In one embodiment, the protein of interest is a tumor necrosis factor (TNF) superfamily (TNFSF) TNF homology domain (THD). In one embodiment, a single chain polypeptide muti-effector domain may comprise the following domain organization: N-terminus-linker-THD-linker-THD-linker-THD-C-terminus. For example, the THD is from a TNF superfamily member (TNFSF) selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, TWEAK, or a fragment thereof. The N-terminus of the single chain polypeptide muti-effector domain may be covalently linked via a peptide bond to a linker polypeptide (for example an Fc or Fc mutein linker) or to a vesicle targeting domain (e.g., one or more membrane anchoring domains from a POI). The multi-effector domain can interact with a target protein on a target cell, wherein said interaction induces target protein clustering or super clustering, and wherein target protein clustering or super clustering induces activity, stimulation, agonism, or co-stimulation of said target protein. It is understood herein that activation of a target protein (e.g., a target receptor) may refer to agonism of said target protein, wherein agonism of said target protein leads to a cellular response associated with said target protein as known by one of ordinary skill in the art.

As used herein, the term “agonist” refers to an agent that interacts with a specific cellular constituent (i.e., receptor) and elicits an observable positive response. For example, an agonist is a substance that mimics the actions of a neurotransmitter or hormone to produce a response when it binds to a specific receptor in the brain. In some examples, agonists and most natural ligands bind to receptors in their inactive state and quickly induce an active receptor conformation that initiates cell signaling. The active receptor state initiates signaling because of its structural complementariness with coupling proteins that activate signaling pathways, such as G proteins and G protein-coupled receptor kinases. As used herein, the term “induced by” refers to producing a response by an agent (i.e., an agonist) that binds to a specific receptor.

As used herein, the term “antagonist” refers to an agent that interacts with the receptor or any other part of the effector mechanism to inhibit the action of an agonist. Antagonist has no activity of its own. In contrast to the action of an agonist, an antagonist, such as naltrexone, binds to a specific receptor in the brain but does not activate it. Therefore, if an agonist, for example heroin or methadone, is present and activating the receptor, taking naltrexone will counteract the activation, resulting in withdrawal.

As used herein, the terms “specifically bind” and/or “specifically recognize” or “substantially binds” refers to the affinity of a binding molecule for a target molecule (e.g., target protein or target receptor) compared to the binding molecule's affinity for non-target molecules. A binding molecule (e.g., a POI domain) that specifically binds a target molecule (e.g., a target protein provided herein) does not substantially recognize or bind non-target molecules. e.g., an antibody “specifically binds” and/or “specifically recognize” another molecule, meaning that this interaction is dependent on the presence of the binding specificity of the molecule structure, e.g., an antigenic epitope. As used herein, “non-specific binding” and “background binding” refers to the interaction that does not depend on the presence of specific structure (e.g., a specific antigenic epitopes). Methods of measuring binding of a polypeptide to a target are known in the art (e.g., differential scanning calorimetry, isothermal titration calorimetry, spectroscopy, crystallography, surface plasmon resonance, co-immunoprecipitation, pulldown assays, crosslinking, yeast two-hybrid system, tandem affinity purification-mass spectroscopy, protein microarrays, bio-layer interferometry, far-Western blots, computational prediction, analytical ultracentrifugation, light scattering, fluorescence spectroscopy, resonance energy transfer, ELISA or ELISPOT assays, or any other assays known by one of ordinary skill in the art).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with an infection or a cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, for example, administration of a composition or construct as described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g., a subject who is not treated with the methods or compositions described herein).

As used herein, the terms “autoimmune condition” and “autoimmune disease” are used interchangeably and refer to any disease characterized by abnormal functioning of the immune system and may include, but is not limited to, achalasia, Addison's disease, adult Still's disease, agammaglobulinemia, alopecia areata, amyloidosis, ankylosing spondylitis, anti-GBM/Anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease, celiac disease, Chagas disease, chronic inflammatory demyelinating polyneuropathy (CIDP), chronic recurrent multifocal osteomyelitis (CRMO), Churg-Strauss syndrome (CSS), eosinophilic granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), Herpes gestationis or pemphigoid gestationis (PG), hidradenitis suppurativa (HS) (acne inversa), hypogammalglobulinemia, IgA nephropathy, IgG4-related sclerosing disease, immune thrombocytopenia purpura (ITP), inclusion body myositis (IBM), interstitial cystitis (IC), juvenile arthritis, type 1 diabetes, juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, Lyme disease chronic, Meniere's disease, microscopic polyangiitis (MPA), mixed connective tissue disease (MCTD), Mooren's ulcer, Mucha-Habermann disease, multifocal motor neuropathy (MMN) or MMNCB, multiple sclerosis, myasthenia gravis, myositis, narcolepsy, neonatal Lupus, neuromyelitis optica, neutropenia, ocular cicatricial pemphigoid, optic neuritis, palindromic rheumatism (PR), PANDA, paraneoplastic cerebellar degeneration (PCD), paroxysmal nocturnal hemoglobinuria (PNH), Parry Romberg syndrome, pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm & testicular autoimmunity, stiff person syndrome (SPS), subacute bacterial endocarditis (SBE), Susac's syndrome, sympathetic ophthalmia (SO), takayasu's arteritis, temporal arteritis/Giant cell arteritis, thrombocytopenia purpura (TTP), Tolosa-Hunt syndrome (THS), transverse myelitis, type 1 diabetes, ulcerative colitis (UC), undifferentiated connective tissue disease (UCTD), uveitis, vasculitis, vitiligo, Vogt-Koyanagi-Harada disease. An autoimmune condition or autoimmune diseases may be caused by, but not limited to, a natural predisposition, an infection (e.g., bacteria or virus), drugs, vaccination, environmental triggers (e.g., toxins or chemicals such as dust, silica, oil, benzene, tri- or per-chloroethylene etc.), stress, cancer, blood or tissue or organ transplantation, or unknown etiology. Autoimmune disorders may result in but not limited to the destruction of body tissue, abnormal growth of an organ or tissue, changes in organ or tissue function (e.g., changes in blood vessels, connective tissue, function of endocrine glands, joints, muscles, blood cells, skin, etc.).

As used herein, the term “cancer” refers to a hyperproliferation of cells that exhibit a loss of normal cellular control that results in unregulated growth, lack of differentiation, local tissue invasion, and metastasis. The methods and compositions described herein can be used for the treatment of solid tumors (e.g., cancer) or non-solid tumors, such as leukemia, blood cell cancers, and the like. Solid tumors can be found in bones, muscles, the brain, or organs, and can be sarcomas or carcinomas. Where the methods and compositions described herein can overcome barriers of tumor treatment, including, but not limited to barriers to treatment or inhibition of metastases, it is contemplated that aspects of the technology described herein can be used to treat all types of solid and non-solid tumor cancers, including cancers not listed in the instant specification. The compositions and methods described herein, without limitation, include methods of treating cancer, methods of inhibiting metastases, and methods of inducing an anti-tumor immune response.

As used herein, the terms “subject”, “individual”, “host”, and “patient” are used interchangeably and may refer to any animal, mammal, bird, fish, reptile, and amphibian, for example, human, monkey, dog, cat, horse, pig, cattle, ox, donkey, rabbit, sheep, goat, mouse, rat, guinea pig, llama, chicken, goose, duck, turkey, or the like receiving or registered to receive a therapeutic amount of a composition of the present invention for medical care or treatment.

As used herein, the term “injection” refers to any process or method which allows the person skilled in the art to administer any therapeutic to a target site by penetration. Examples of injection are, but not limited to, subcutaneous, subcuticular, subcapsular, subarachnoid, intradermal, intramuscular, intravenous, intra-arterial, intraventricular, intracapsular, intraorbital, intraocular, intrathoracic, intraperitoneal, intravitreal, retro-orbital, intranasal, intracerebral, intrathymic, intraspinal, intrasternal, intra-articular, intracavernous, intracardiac, intraosseous, intrathecal, transtracheal, epidural, or the like as known in the art. A therapeutic does of the present invention may be delivered to a patient by way of controlled release, for example but not limited to, implantable pump and implantable cannulas to provide continuous access to the venous or arterial system.

As used herein, the term “topical application” refers to applying or spreading a composition of the present invention onto surfaces on or in the body, both internally and/or externally, in a therapeutically effective amount for local and/or systemic treatment. Topical application may be epicutaneuos wherein a composition of the present invention may be directly applied onto a localized surface of the skin or mucous membranes. Topical application may include transdermal application wherein a composition of the present invention may be absorbed into the body to obtain systemic delivery and systemic distribution. For example, a transdermal patch may be applied onto the body to deliver a therapeutic dose of a composition of the invention presented herein. Topical application formulations may include, but are not limited to, creams, foams, gels, lotions, solutions, ointments, dermal patch, transdermal patches, powder, solid, sponge, tape, vapor, paste, film, liposomes, balm, shampoo, spray, or tincture. A therapeutic dose of a composition of the present invention may be delivered vaginally (for example a vaginal suppository, vaginal ring, douche, intrauterine device, intravesical infusion, and the like) or urethra.

As used herein, the term “enteral administration” refers to a composition of the present invention administered via the gastrointestinal tract in a therapeutically effective amount for local or systemic treatment. Enteral administration may include, but is not limited to, delivery of a composition of the present invention via the mouth, sublingual, esophagus, gastric (for example the stomach), small intestines, large intestines or rectum. Oral delivery of the present invention may include, but is not limited to, the use of a capsule, pastille, pill, tablet, solution, gel, suspension, emulsion, syrup, elixir, tincture, mouthwash, lozenges, chewing gum, lollipop, osmotic-controlled release oral delivery system, or the like. Gastric delivery may involve the use of a tube or nasal passage that leads directly to the stomach, for example, a percutaneous endoscopic gastrostomy tube. Gastric delivery may involve direct injection made through the abdominal wall. Rectal delivery may involve, but is not limited to, the use of a suppository, ointment, enema, murphy drip, or the like. A therapeutic does of the present invention may be delivered to a patient by way of controlled release, for example but not limited to, controlled release drug delivery pellet or pill.

As used herein, the terms “pulmonary system” or “respiratory system” are used interchangeably and refer, but are not limited, to the respiratory region, conducting airways, nasal cavity, sinuses, nasopharynx, oropharynx, larynx, trachea, bronchi, bronchioles, respiratory bronchioles, alveolar ducts, alveolar sacs, respiratory epithelium (e.g., alveolar epithelial cells), endothelial cells, or the like.

As used herein, the terms “pulmonary delivery” and “pulmonary administration” are used interchangeably and refer to delivering a composition of the present invention to the respiratory system through the respiratory route, including but not limited to, intranasal administration, oral administration, and oral inhalative administration (e.g., intratracheal instillation and intratracheal inhalation) of a therapeutically effective amount for local or systemic treatment. Pulmonary delivery of a therapeutically effective amount of a composition of the present invention may be achieved by dispersion, for example by using a syringe. Pulmonary delivery of a composition of the present invention may be achieved by aerosol administration, wherein aerosol administration may deposit a therapeutically effective amount of the present invention by gravitational sedimentation, inertial impaction, or diffusion.

Pulmonary delivery of a therapeutically effective amount of a composition of the present invention may be deposited on any mucus layer of the respiratory system, for example, but not limited to, the mucus layer which coats the walls of conducting airways, the smaller airway, and/or alveolar space.

As used herein, an “appropriate control” refers to an untreated, otherwise identical cell or population (e.g., a subject who was not administered the composition described herein, or was administered by only a subset of agents provided herein, as compared to a non-control cell).

As used herein, a “reference level” can refer to one or more parameters or markers as measured for a normal, otherwise unaffected cell population or tissue (e.g., a biological sample obtained from a healthy subject, or a biological sample obtained from the subject at a prior time point, or a biological sample that has not yet been contacted with a pathogen as described herein). For measuring or monitoring therapeutic efficacy, a level determined prior to treatment or earlier in treatment can also provide a reference level for a given parameter or value.

As used herein, the term “modulates” refers to an effect including increasing or decreasing a given parameter as those terms are defined herein.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level. For example, increasing activity can refer to activating a receptor or a signaling pathway (e.g., antibody production or inflammation).

The terms “decrease”, “reduced”, “reduction”, or “inhibit” are all used herein to mean a decrease or lessening of a property, level, or other parameter by a statistically significant amount. In some embodiments of any of the aspects, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein the term “post translation modification” is used herein to mean amino acid side chain modification in some proteins after their biosynthesis. Protein post translation modifications (PTMs) increase the functional diversity of the proteome by the covalent addition of functional groups or proteins, proteolytic cleavage of regulatory subunits, or degradation of entire proteins. There are more than 400 different types of PTMs affecting many aspects of protein functions. Such modifications happen as crucial molecular regulatory mechanisms to regulate diverse cellular processes. These modifications include phosphorylation, glycosylation, ubiquitination, nitrosylation, methylation, acetylation, lipidation and proteolysis and influence almost all aspects of normal cell biology and pathogenesis. These processes have a significant impact on the structure and function of proteins. Disruption in PTMs can lead to the dysfunction of vital biological processes and hence to various diseases.

As used herein the term “farnesylation” is used herein to mean one of the important steps in the posttranslational modification of proteins associated with intracellular signal transduction. The process is catalyzed by a heterodimeric zinc protein known as farnesyltransferase, and involves the transfer of a farnesyl group from farnesylpyrophosphate to the C-terminal cysteine sulfur of the target proteins such as Ras. These protein substrates, in general, have characteristic C-terminal consensus sequences of the type CAAX (C, cysteine; A, aliphatic amino acid; X, serine or methionine) and prenylation is important for their translocation to the membrane where they form part of the signaling network. Since constitutive activation of Ras is a major contributory factor in a number of malignant human tumors, its inactivation by interfering with the farnesylation step has been extensively studied as a strategy to develop new anticancer agents. Farnesylation facilitates their membrane association and also promotes protein-protein interaction.

As used herein the term “lipid nanoparticles” refers to nanoparticles composed of lipids. They are a pharmaceutical drug delivery system, and a pharmaceutical formulation. In some aspects, lipid nanoparticles are spherical vesicles made of ionizable lipids, which are positively charged at low pH (enabling RNA complexation) and neutral at physiological pH (reducing potential toxic effects, as compared with positively charged lipids, such as liposomes).

Lipid nanoparticles (LNPs) are vehicles for gene delivery that accommodate both nucleic acid and protein. The delivery of therapeutic proteins to the body can be challenging. Lipid nanoparticles overcome the challenges of therapeutic proteins delivery, and different methods are used to tune protein-loaded lipid nanoparticles features. For example, fusion proteins may be efficiently conjugated to the surface of lipid nanoparticles for selective presentation and delivery of the targeting domains, signaling domains or both.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

The abbreviation, “etc.” is derived from the Latin et cetera, and is used herein to indicate a non-limiting list. Thus, the abbreviation “etc.,” is synonymous with the term “and other similar things”, or “and so forth”.

Other than in the operating examples, or where otherwise indicated, all numbers expressing quantities of ingredients or reaction conditions used herein should be understood as modified in all instances by the term “about.” The term “about” when used in connection with percentages can mean ±1%.

The term “statistically significant” or “significantly” refers to statistical significance and generally means a two-standard deviation (2SD) difference, above or below a reference value. Additional definitions are provided in the text of individual sections below.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. The term “or” is inclusive unless modified, for example, by “either.” Other than in the operating examples, or where otherwise indicated, all numbers expressing quantities of ingredients or reaction conditions used herein should be understood as modified in all instances by the term “about.”

Unless otherwise explained, all technical and scientific terms used herein have the same meaning as commonly understood by one of ordinary skill in the art to which this disclosure belongs.

It is to be understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES AND DETAILED DESCRIPTIONS OF THE DRAWINGS

The following examples are provided by way of illustration, not limitation.

### Example 1. Identification of Extracellular Vesicle-Targeted Proteins

Flp-In 293 cells (Invitrogen) were grown in DMEM 10% FBS and subsequently washed 3× in DPBS and then incubated in basal DMEM for 2 days. The serum-free conditioned media from was harvested and filtered through a 0.45 μm PES filter. The filtered media was concentrated and buffer-exchanged to Dulbecco's phosphate buffered saline (DPBS) using an Amicon Ultra-15 Centrifugal Filter with a 10 kDa molecular weight cutoff. Extracellular vesicles were separated and purified from smaller biomolecules using qEVoriginal/35 nm size exclusion chromatography (SEC) columns (Izon Science) essentially according to the manufacturer's instructions the contents of which are incorporated herein by reference in their entirety (support.izon.com/qev-columns #user-guides). Exosome containing fractions were pooled and proteins were precipitated with trichloroacetic acid, washed with acetone twice, dried, and stored at −20° C. until further processing. Protein samples were resuspended in 8M urea in 100 mM Tris pH 8.5, reduced, alkylated and digested by the sequential addition of lys-C and trypsin proteases as previously described. The digested peptide solution was fractionated online using strong-cation exchange and reverse phase chromatography and eluted directly into an LTQ-Orbitrap mass spectrometer (ThermoFisher). MS/MS spectra were collected and subsequently analyzed using the ProLuCID and DTASelect algorithms. Database searches were performed against a human database containing the relevant paramyxovirus M protein sequence. Protein and peptide identifications were further filtered with a false positive rate of less than 5% as estimated by a decoy database strategy. Normalized spectral abundance factor (NSAF) values were calculated as described.

Table 2A shows the top 100 identified proteins ordered by NSAF multiplied by 10⁵. To identify exosome membrane proteins that are also expressed on the cell surface, the identified proteins were filtered using SURFY, the in silico surfaceome. This public resource of 2,886 proteins can be used to query proteotype and transcriptomic datasets for context-dependent biomarkers and drug-target candidates. Table 2B shows the top 50 exosome proteins filtered using SURFY. Combined, Tables 2A and 2B demonstrate that the two most abundant Type II membrane proteins on exosomes are CD298 and CD98 heavy chain (4F2 protein).

### Example 2. Design of Engineered Extracellular Vesicles

As described, engineered extracellular vesicles are engineered to induce and propagate biological signaling, including for example, antagonist and agonist signaling. Engineered extracellular vesicles are designed to include hallmark biophysical and biochemical features of cell derived extracellular vesicles, further including vesicle targeting domains and signaling domains. Vesicle targeting domains capable of attaching to extracellular vesicles such as exosomes, signaling domains, optionally including a linker (e.g., Fc linker), can be organized in genetic vector constructs. Engineered extracellular vesicles are designed to include hallmark biophysical and biochemical features of cell derived extracellular vesicles, further including at least one fusion polypeptide, wherein said fusion polypeptide further comprises a vesicle targeting domain and a signaling domain (e.g., one or more signaling domain from one or more protein of interest). Fusion polypeptides capable of attaching or anchoring to extracellular vesicles such as exosomes, comprise vesicle targeting domains, signaling domains, and optionally including a linker (e.g., Fc linker), can be organized in genetic vector constructs.

According to some embodiments, an engineered extracellular vesicle is designed to express one or more engineered fusion polypeptides to induce agonistic receptor signaling through binding and clustering. For example, the engineered fusion polypeptide includes multi-effector domain(s), multimerization linker (for example, Fc/Fc mutein), and bilayer targeting (vesicle targeting domain).

An engineered extracellular vesicle is designed to express either a Type I transmembrane fusion polypeptide or a Type II transmembrane fusion polypeptide. In some embodiments, an engineered extracellular vesicle is constructed to express a fragment of a Type I transmembrane protein, or a multi-pass transmembrane protein or fragment thereof. For example, the multi-pass transmembrane protein is a tetraspanin or fragment thereof. In some embodiments, an engineered extracellular vesicle is constructed to express a fragment of the Type II transmembrane protein, or a multi-pass transmembrane protein or fragment thereof. For example, the multi-pass transmembrane protein is a tetraspanin or fragment thereof.

In some embodiments, the engineered extracellular vesicle includes at least one fusion polypeptide, the fusion polypeptide including an agonistic multi-effector domain in an exterior position relative to a phospholipid bilayer of the extracellular vesicle; a vesicle targeting domain spanning at least partly through the phospholipid bilayer of the extracellular vesicle; and a polypeptide linker positioned between the agonistic multi-effector domain and the at least one vesicle targeting domain. For example, the polypeptide linker is positioned N-terminus relative to the at least one vesicle targeting domain. For example, the polypeptide linker is positioned C-terminus relative to the agonistic multi-effector domain. For example, the vesicle targeting domain includes a secretion signal recognition sequence. For example, the secretion signal recognition sequence is positioned N-terminus relative to the agonistic multi-effector domain. For example, the secretion signal recognition sequence is proteolytically cleaved from the agonistic multi-effector domain.

In some embodiments, the engineered extracellular vesicle includes at least one fusion polypeptide, the fusion polypeptide including an agonistic multi-effector domain in an exterior position relative to a phospholipid bilayer of the extracellular vesicle; a vesicle targeting domain spanning at least partly through the phospholipid bilayer of the extracellular vesicle; and a polypeptide linker positioned between the agonistic multi-effector domain and the at least one vesicle targeting domain. For example, the vesicle targeting domain includes a myristoylation and/or palmitoylation motif. For example, the polypeptide linker is positioned C-terminus relative to the at least one vesicle targeting domain. For example, the polypeptide linker is positioned N-terminus relative to the agonistic multi-effector domain.

### Schematic of Type II Fusion Polypeptide.

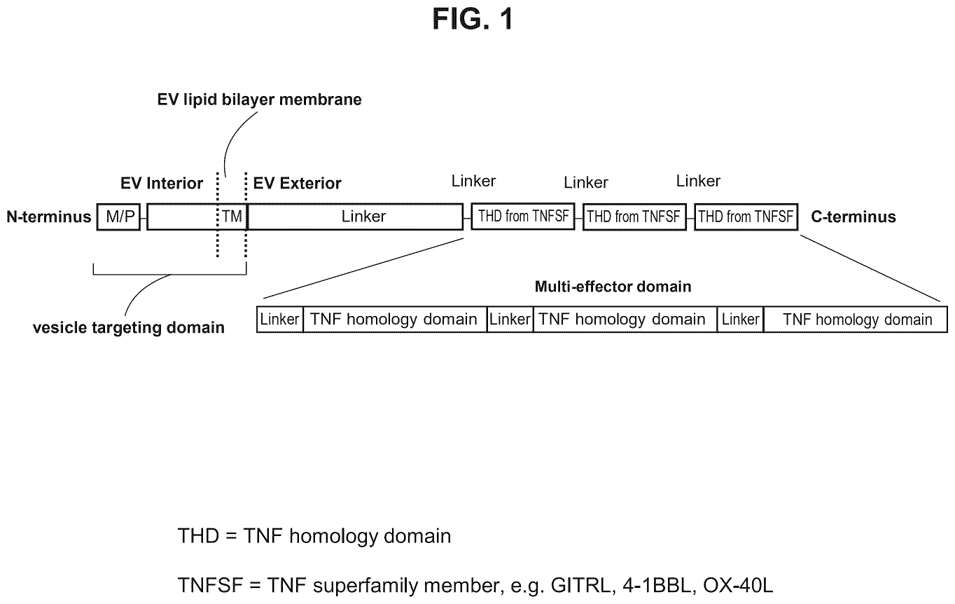

FIG. 1 shows a schematic diagram linear representation of a type II membrane fusion polypeptide comprising a vesicle targeting domain, a multi-effector domain, and a plurality of linkers. The N-Terminus being on the interior of the extracellular vesicle while the C-Terminus being on the exterior. The vesicle targeting domain comprises a myristoylation and palmitoylation polypeptide sequence (M/P) and a sequence comprising a transmembrane sequence (TM). The fusion polypeptide optionally comprises a linker (e.g. an immunoglobulin Fc or Fc mutein). The multi-effector domain may comprise at least one or a plurality of protein of interest, or active fragments thereof, joined by linkers. As shown in FIG. 1, in a preferred embodiment the multi-effector domain may comprise at least one or a plurality of tumor necrosis factor (TNF) superfamily (TNFSF) TNF homology domains (THD) joined by linkers. In a preferred embodiment the multi-effector domain of the fusion polypeptide comprises three TNF homology domains joined by linkers.

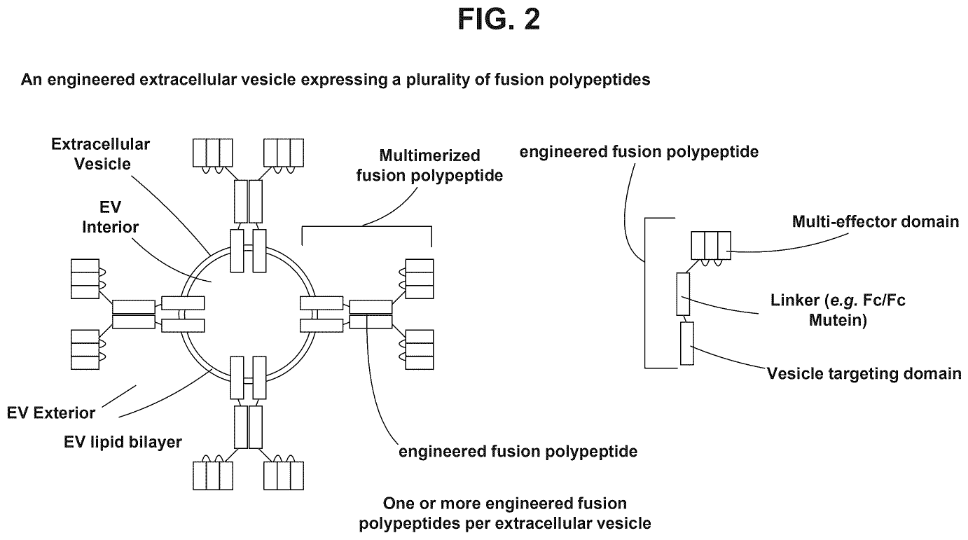

FIG. 2 is a diagram of an engineered extracellular vesicle displaying one or more engineered fusion polypeptides anchored in a lipid bilayer of the extracellular vesicle and the ectodomain of the fusion polypeptide is displayed on the surface of the engineered extracellular vesicle according to various embodiments of the present invention. The engineered extracellular vesicle may display one or more engineered fusion polypeptides according to various embodiments of the present invention. A fusion polypeptide may multimerize with a second fusion polypeptide via homotypic interaction of the Fc or Fc mutein domain or the interactions between the signaling effector domain. In other embodiments, a fusion polypeptide may multimerize with more than one other fusion polypeptide via interaction. In a preferred embodiment, the multi-effector domain may form a single chain multimer of the repeated monomers. The multi-effector domain substantially induces agonist receptor signaling through binding and clustering of a target receptor. In a preferred embodiment the multi-effector domain of the fusion polypeptide comprises at least three proteins of interest or active fragments joined by linkers, wherein the three proteins of interest or active fragments joined by linkers substantially induce agonist signaling of a target receptor through binding and clustering of said target receptor. In other embodiments the multi-effector domain of the fusion polypeptide comprises more than three proteins of interest or active fragments joined by linkers, wherein the more than three proteins of interest or active fragments joined by linkers substantially induce agonist signaling of a target receptor through binding and clustering of said target receptor. It is understood by one of ordinary skill in the art that FIG. 2 may be a representation of any fusion polypeptide as provided herein wherein the N-Terminus may be on the interior of the extracellular vesicle while the C-Terminus being on the exterior (for example a type II membrane protein fusion polypeptide), or the C-Terminus being on the interior of the extracellular vesicle while the N-Terminus being on the exterior (for example a type I membrane protein fusion polypeptide).

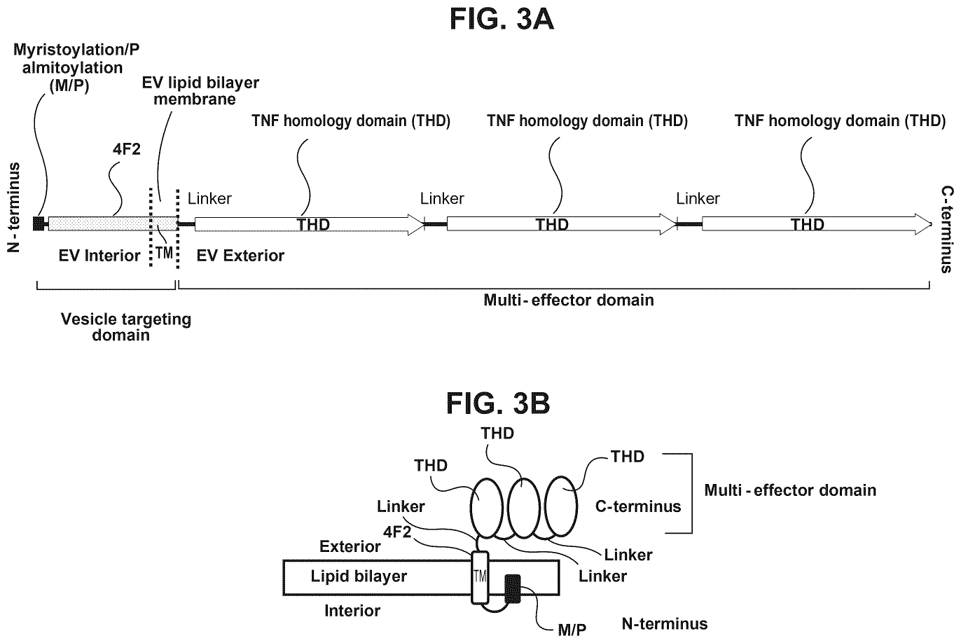

FIG. 3A is a schematic diagram linear representation of a type II membrane protein fusion polypeptide comprising a 4F2 vesicle targeting domain further comprising a transmembrane domain, a multi-effector domain, and a plurality of linkers. The N-Terminus of the fusion polypeptide being on the interior of the extracellular vesicle while the C-Terminus being on the exterior. The fusion polypeptide comprises membrane anchoring myristoylation and palmitoylation (M/P) moiety, a membrane anchoring sequence from 4F2 heavy chain protein further comprising a transmembrane domain, and a multiplicity of tumor necrosis factor (TNF) superfamily (TNFSF) TNF homology domains (THD) joined by polypeptide linkers. FIG. 3B shows a Type II membrane protein fusion polypeptide embedded in a lipid bilayer and displayed on the surface of an engineered extracellular vesicle comprising a multi-effector domain, a 4F2 vesicle targeting domain further comprising a transmembrane domain, and a myristoylation and palmitoylation membrane anchoring sequence, and a plurality of linkers, the C-Terminus being on the exterior of the exosome membrane while the N-Terminus being on the interior. The fusion polypeptide comprises a myristoylation and palmitoylation sequence, a sequence from 4F2 heavy chain protein, and a multiplicity of tumor necrosis factor (TNF) superfamily (TNFSF) TNF homology domains (THD) joined by polypeptide linkers.

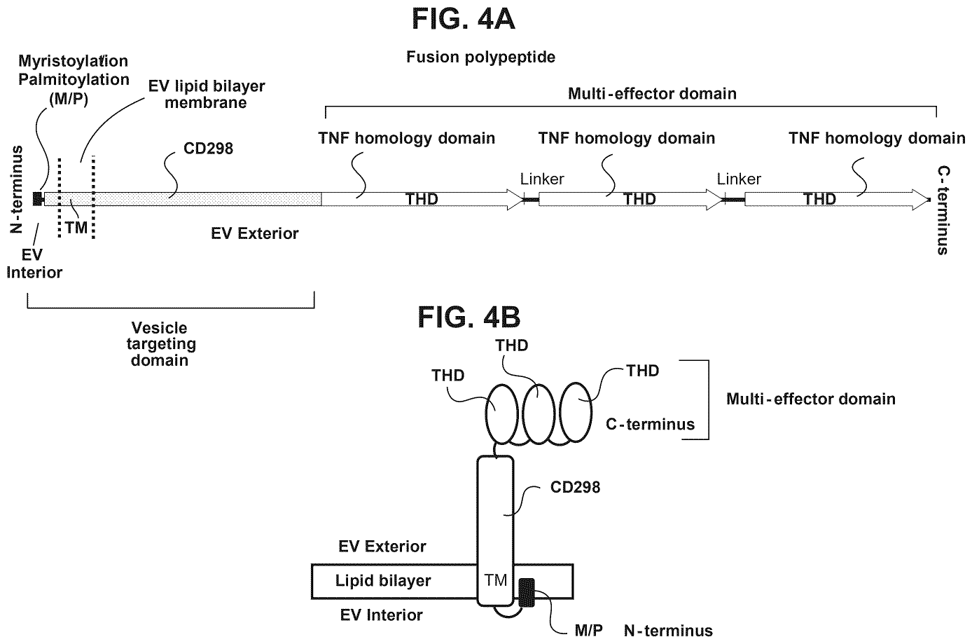

FIG. 4A is a schematic diagram linear representation of a type II membrane protein fusion polypeptide comprising a CD298 vesicle targeting domain further comprising a transmembrane domain, a multi-effector domain, and a plurality of linkers. The N-Terminus of the fusion polypeptide being on the interior of the extracellular vesicle while the C-Terminus being on the exterior. The fusion polypeptide comprises membrane anchoring myristoylation and palmitoylation (M/P) moiety, a membrane anchoring sequence from CD298 protein further comprising a transmembrane domain, and a multiplicity of tumor necrosis factor (TNF) superfamily (TNFSF) TNF homology domains (THD) joined by polypeptide linkers. FIG. 4B shows a Type II membrane protein fusion polypeptide embedded in a lipid bilayer and displayed on the surface of an engineered extracellular vesicle comprising a multi-effector domain, a CD298 vesicle targeting domain further comprising a transmembrane domain, and a myristoylation and palmitoylation membrane anchoring sequence, and a plurality of linkers, the C-Terminus being on the exterior of the exosome membrane while the N-Terminus being on the interior. The fusion polypeptide comprises a myristoylation and palmitoylation sequence, a sequence from CD298 protein, and a multiplicity of tumor necrosis factor (TNF) superfamily (TNFSF) TNF homology domains (THD) joined by polypeptide linkers.

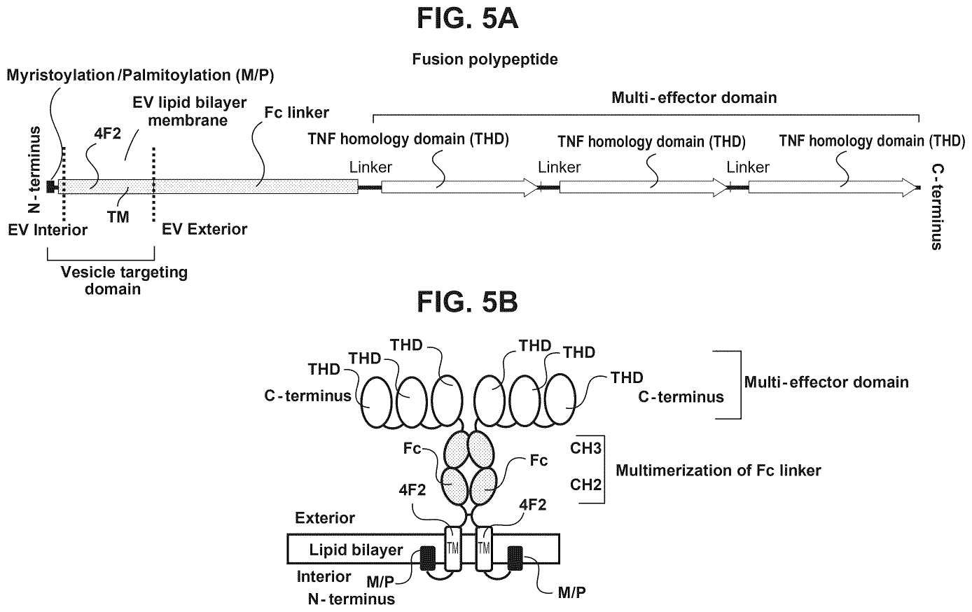

FIG. 5A is a schematic diagram linear representation of a type II membrane protein fusion polypeptide comprising a signaling multi-effector domain, a vesicle targeting domain wherein the vesicle targeting domain is a sequence from 4F2 heavy chain (e.g., a transmembrane sequence from 4F2), and a plurality of linkers wherein one of the linkers is an Fc or an Fc mutein linker, and a myristoylation and palmitoylation sequence, the C-Terminus being on the exterior of an extracellular vesicle membrane while the N-Terminus being on the interior. The fusion polypeptide comprises a myristoylation and palmitoylation sequence, a sequence from 4F2 protein further comprising a transmembrane domain, and a multiplicity of tumor necrosis factor (TNF) superfamily (TNFSF) TNF homology domains (THD) joined by polypeptide linkers, and further comprising an Fc linker wherein the Fc linker may be an Fc mutein. FIG. 5B shows a Type II membrane protein fusion polypeptide embedded in a lipid bilayer and displayed on the surface of an engineered extracellular vesicle comprising a signaling multi-effector domain, a vesicle targeting domain wherein the vesicle targeting domain is a sequence from 4F2 heavy chain (e.g., a transmembrane sequence from 4F2), and a plurality of linkers wherein one of the linkers is an Fc or an Fc mutein linker, and a myristoylation and palmitoylation sequence, the C-Terminus being on the exterior of an extracellular vesicle membrane while the N-Terminus being on the interior. The fusion polypeptide comprises a myristoylation and palmitoylation sequence, a sequence from 4F2 protein further comprising a transmembrane domain, and a multiplicity of tumor necrosis factor (TNF) superfamily (TNFSF) TNF homology domains (THD) joined by polypeptide linkers, and further comprising an Fc linker wherein the Fc linker may be an Fc mutein.

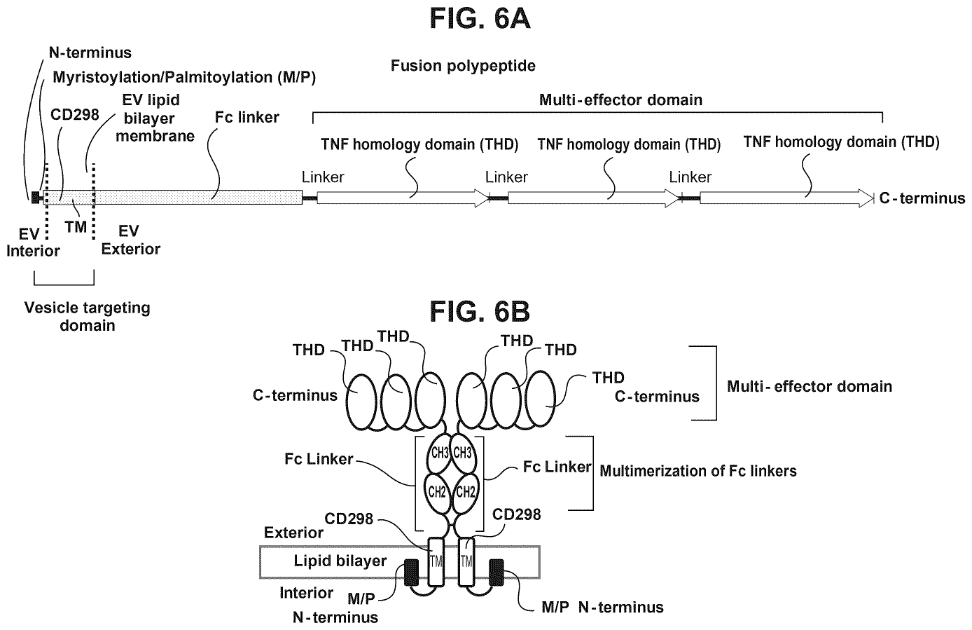

FIG. 6A is a schematic diagram linear representation of a type II membrane protein fusion polypeptide comprising a signaling multi-effector domain, a vesicle targeting domain wherein the vesicle targeting domain is a sequence from CD298 (e.g., a transmembrane sequence from CD298), and a plurality of linkers wherein one of the linkers is an Fc or an Fc mutein linker, and a myristoylation and palmitoylation sequence, the C-Terminus being on the exterior of an extracellular vesicle membrane while the N-Terminus being on the interior. The fusion polypeptide comprises a myristoylation and palmitoylation sequence, a sequence from CD298 protein further comprising a transmembrane domain, and a multiplicity of tumor necrosis factor (TNF) superfamily (TNFSF) TNF homology domains (THD) joined by polypeptide linkers, and further comprising an Fc linker wherein the Fc linker may be an Fc mutein. FIG. 6B shows a Type II membrane protein fusion polypeptide embedded in a lipid bilayer and displayed on the surface of an engineered extracellular vesicle comprising a signaling multi-effector domain, a vesicle targeting domain wherein the vesicle targeting domain is a sequence from CD298 (e.g., a transmembrane sequence from CD298), and a plurality of linkers wherein one of the linkers is an Fc or an Fc mutein linker, and a myristoylation and palmitoylation sequence, the C-Terminus being on the exterior of an extracellular vesicle membrane while the N-Terminus being on the interior. The fusion polypeptide comprises a myristoylation and palmitoylation sequence, a sequence from CD298 protein further comprising a transmembrane domain, and a multiplicity of tumor necrosis factor (TNF) superfamily (TNFSF) TNF homology domains (THD) joined by polypeptide linkers, and further comprising an Fc linker wherein the Fc linker may be an Fc mutein.

### Example 3. Genetic Constructs

In some embodiments, for an engineered extracellular vesicle including a Type I transmembrane protein or fragment thereof, or a multi-pass transmembrane protein or fragment thereof, the multi-pass transmembrane protein is a tetraspanin or fragment thereof. For example, the tetraspanin is CD9 or fragment thereof. For example, the fragment of CD9 is CD9tm2. For example, palmitoylation/farnesylation modification is included in the multi-pass transmembrane protein.

In some embodiments, for an engineered extracellular vesicle including a Type II transmembrane protein or fragment thereof, or a multi-pass transmembrane protein or fragment thereof, the transmembrane protein includes 4F2. For example, myristoylation/palmitoylation modification is included in the multi-pass transmembrane protein.

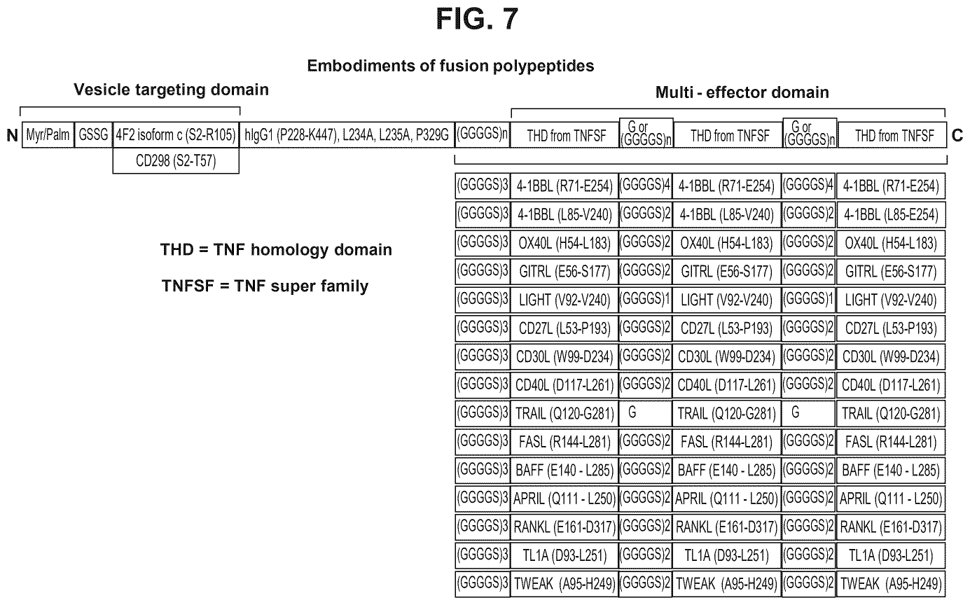

FIG. 7 shows a diagram linear representation of the domain organization of type II transmembrane protein fusion polypeptide embodiments comprising a vesicle targeting domain, a multi-effector domain, and a plurality of linkers and an Fc (e.g., Fc mutein) polypeptide linker. The N-Terminus (N) being on the interior of the extracellular vesicle while the C-Terminus (C) being on the exterior. FIG. 7 shows schematic diagrams of various type II transmembrane protein fusion polypeptide embodiments that may be displayed on an engineered extracellular vesicle.

Referring to FIG. 7, the Type II transmembrane protein fusion polypeptide includes a vesicle targeting domain comprising at least one membrane anchoring domain, for example a transmembrane domain sequence from 4F2 heavy chain (e.g., S2-R105 in SEQ ID NO: 12) and a myristoylation and palmitoylation membrane anchoring domain (e.g., SEQ ID NO: 17 or SEQ ID NO: 18, nucleic acid sequence and protein sequence, respectively). In some embodiments of the fusion polypeptide's vesicle targeting domain, the 4F2 heavy chain membrane anchoring domain and the myristoylation/palmitoylation membrane anchoring domain are connected by a linker.

Referring to FIG. 7, in some embodiments, The Type II transmembrane protein fusion polypeptide includes a vesicle targeting domain comprising at least one membrane anchoring domain, for example a transmembrane domain sequence from CD298 (e.g., S2-T57 in SEQ ID NO: 14) and a myristoylation and palmitoylation membrane anchoring domain (e.g., SEQ ID NO: 17 or SEQ ID NO: 18, nucleic acid sequence and protein sequence, respectively). In some embodiments of the fusion polypeptide's vesicle targeting domain, the CD298 membrane anchoring domain and the myristoylation/palmitoylation membrane anchoring domain are connected by a linker.

In various embodiments, the fusion polypeptide further includes a linker between the agonistic multi-effector domain and the Fc or Fc mutein polypeptide linker. In some embodiments, the fusion polypeptide displayed on an extracellular vesicle further includes a linker between the polypeptide linker and the vesicle targeting domain. In some embodiments, the engineered extracellular vesicle fusion polypeptide further includes a linker between each POI fragment of the agonistic multi-effector domain. For example, the agonistic multi-effector domain comprises three agonistic signaling domain fragments selected from a protein of interest. For example, each individual agonistic POI fragment of the agonistic multi-effector domain is a tumor necrosis factor (TNF) homology domain (THDs) or a fragment thereof. For example, the THD is from a TNF superfamily member (TNFSF) selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, TWEAK, or a fragment thereof. The linker between the agonistic multi-effector domain and the polypeptide linker, the linker between the polypeptide linker (e.g., Fc or Fc mutein linker) and the vesicle targeting domain, or the linker between individual agonistic POI fragments in the multi-effector domain is each independently selected from the group consisting of ID, GSSG (SEQ ID NO:154), G, GS, (G)n, GG, GGS, GGG, GGGS (SEQ ID NO: 218), (GGGS)n, GGGG (SEQ ID NO:219), GGGGS (SEQ ID NO: 156), (GGGGS)n, wherein n is an integer between 1 and 10, and combinations thereof. In some embodiments n can be an integer greater than 10.

In some embodiments, a CVIM motif from KRAS-13 (which is a “CAAX box”) is added at the C-terminus of the fusion polypeptide. For example, the motif is farnesylated (a type of prenylation) which is a post translational modification, wherein an isoprenyl group is added to the cysteine residue, and the VIM is cleaved via proteolysis.

Referring to FIG. 7, the vesicle targeting domain of the fusion polypeptide comprises a myristoylation and palmitoylation polypeptide sequence (Myr/Palm) and further comprises a 4F2 heavy chain fragment or a fragment of CD298. The fusion polypeptide optionally comprises an immunoglobulin Fc or Fc mutein linker. The multi-effector domain may comprise at least one or a plurality of protein of interest or fragments thereof. The multi-effector domain may be an agonist to a target receptor. The multi-effector domain may comprise at least one or a plurality of tumor necrosis factor (TNF) superfamily (TNFSF) TNF homology domains (THD) joined by linkers. In a preferred embodiment the multi-effector domain of the fusion polypeptide comprises at three or more TNF homology domains joined by linkers. For example, the protein of interest (POI) is selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, TWEAK or active fragments thereof, joined by linkers. For example, the linker between the agonistic multi-effector domain and the polypeptide linker (e.g., Fc or Fc mutein linker), the linker between the polypeptide linker and vesicle targeting domain, or the linker between individual agonistic POI fragments in the multi-effector domain is each independently selected from the group consisting of ID, GSSG (SEQ ID NO: 154), G, GS, (G) n, GG, GGS, GGG, GGGS (SEQ ID NO: 218), (GGGS)n, GGGG (SEQ ID NO: 219), GGGGS (SEQ ID NO: 156), (GGGGS) n, wherein n is an integer between 1 and 10, and combinations thereof. In some embodiments n can be an integer greater than 10.

In a preferred embodiment, the fusion polypeptide comprising 4-1BBL multi-effector domain is (SEQ ID NO: 175). In another embodiment, the fusion polypeptide comprising 4-1BBL multi-effector domain is (SEQ ID NO: 203). In another embodiment, the fusion polypeptide comprising 4-1BBL multi-effector domain is (SEQ ID NO: 205). In another embodiment, the fusion polypeptide comprising 4-1BBL multi-effector domain is (SEQ ID NO: 207). In another embodiment, the fusion polypeptide comprising 4-1BBL multi-effector domain is (SEQ ID NO: 209). In another embodiment, the fusion polypeptide comprising 4-1BBL multi-effector domain is (SEQ ID NO: 211). In another embodiment, the fusion polypeptide comprising 4-1BBL multi-effector domain is (SEQ ID NO: 217). In a preferred embodiment, the fusion polypeptide comprising GITRL multi-effector domain is (SEQ ID NO: 177). In a preferred embodiment, the fusion polypeptide comprising GITRL multi-effector domain is (SEQ ID NO: 213). In a preferred embodiment, the fusion polypeptide comprising OX40L multi-effector domain is (SEQ ID NO: 179). In a preferred embodiment, the fusion polypeptide comprising OX40L multi-effector domain is (SEQ ID NO: 215). In a preferred embodiment, the fusion polypeptide comprising CD27L multi-effector domain is (SEQ ID NO: 181). In a preferred embodiment, the fusion polypeptide comprising CD30L multi-effector domain is (SEQ ID NO: 183). In a preferred embodiment, the fusion polypeptide comprising CD40L multi-effector domain is (SEQ ID NO: 185). In a preferred embodiment, the fusion polypeptide comprising LIGHT multi-effector domain is (SEQ ID NO: 187). In a preferred embodiment, the fusion polypeptide comprising TRAIL multi-effector domain is (SEQ ID NO: 189). In a preferred embodiment, the fusion polypeptide comprising RANKL multi-effector domain is (SEQ ID NO: 191). In a preferred embodiment, the fusion polypeptide comprising TL1A multi-effector domain is (SEQ ID NO: 193). In a preferred embodiment, the fusion polypeptide comprising FASL multi-effector domain is (SEQ ID NO: 195). In a preferred embodiment, the fusion polypeptide comprising BAFF multi-effector domain is (SEQ ID NO: 197). In a preferred embodiment, the fusion polypeptide comprising APRIL multi-effector domain is (SEQ ID NO: 199). In a preferred embodiment, the fusion polypeptide comprising TWEAK multi-effector domain is (SEQ ID NO:201). Sequences of all domains are available in the sequence listings. Nucleic acid sequences of all fusion polypeptides are available in the sequence listings.

### Example 4. Purification of Engineered Extracellular Vesicles Displayed with Type I or Type II Membrane Fusion Polypeptides

An exemplary process for purification of the engineered EVs are shown in the flow chart provided in FIG. 38.

### Example 5. Purification and Analysis of Engineered Extracellular Vesicles Displayed with 4-1BB Agonist Fusion Polypeptide Embodiments

FIG. 8 shows an example of results of extracellular vesicle (EV) purification from cellular conditioned medium using size exclusion chromatography (SEC) and characterization of SEC fractions. Referring to FIG. 8A, dot blot analysis was performed on fractions collected during SEC purification of unmodified extracellular vesicles and extracellular vesicles engineered to display various fusion polypeptide embodiments. For example, unmodified Flp-In 293, or Flp-In 293 cells genetically engineered to express native full-length human 4-1BBL or an embodiment of a 4-1BBL fusion polypeptide presented herein (e.g., M/P-4-1BBL, 4F2-4-1BBL, M/P-4F2-4-1BBL, M/P-4F2-Fc (mono)-4-1BBL, or M/P-4F2-Fc-sc4-1BBL), were incubated in serum-free conditioned media within a culture vessel known by one of ordinary skill in the art. The serum-free conditioned media from the cell cultures described herein was harvested and filtered through a 0.45 μm PES filter. The filtered media was concentrated and buffer-exchanged to Dulbecco's phosphate buffered saline (DPBS) using an Amicon Ultra-15 Centrifugal Filter with a 10 kDa molecular weight cutoff. Extracellular vesicles were separated and purified from smaller biomolecules using qEVoriginal/35 nm size exclusion chromatography (SEC) columns (Izon Science) essentially according to the manufacturer's instructions the contents of which are incorporated herein by reference in their entirety (support.izon.com/qev-columns#user-guides). Following the SEC column void volume (fractions 1-6), fractions 7-18 were collected. Fractions 7-18 were blotted onto PVDF-FL membrane under vacuum suction using a 96-well Bio-Dot Microfiltration Apparatus (Bio-Rad) and probed with antibodies to an endogenous extracellular vesicle protein (ALIX) and a component of the fusion polypeptide (4-1BBL), followed by appropriate fluorescent secondary antibodies. The blots were imaged and analyzed using a Li-Cor Odyssey fluorescent imager. Results show SEC fractions 7, 8, and 9 with the highest level of endogenous extracellular vesicle protein ALIX, indicating that fractions 7, 8 and 9 comprise extracellular vesicles. Results show SEC fractions 7, 8, and 9 with the highest level of expression of the fusion polypeptide, indicating that fractions 7, 8 and 9 comprise extracellular vesicles displaying fusion polypeptide embodiments as described herein.

FIG. 8B shows the background subtracted integrated intensity (I.I.) of SEC fraction 8 as determined by using Li-Cor Odyssey software. Total protein concentration of each fraction was measured using fluorometric Qubit protein assays and a Qubit fluorometer (ThermoFisher Scientific). Extracellular vesicles purified from unmodified Flp-In cells did not display a 4-1BBL fusion polypeptide (no integrated intensity observed). Extracellular vesicles purified from Flp-In cells engineered to express native full length 4-1BBL showed moderate integrated intensity (normalized herein to equal one). Extracellular vesicles purified from Flp-In cells engineered to express a 4-1BBL fusion polypeptide showed increased integrated intensity when compared to extracellular vesicles engineered to display the human full length 4-1BBL. These results demonstrate that the 4-1BBL fusion polypeptides displayed at an increased amount compared to native full length 4-1BBL. Extracellular vesicles purified form cells expressing 4-1BBL fusion polypeptide embodiments described herein displayed ALIX protein wherein ALIX protein is an endogenous extracellular vesicle protein, for example an endogenous exosome protein, demonstrating that the purified sample analyzed herein is an extracellular vesicle.

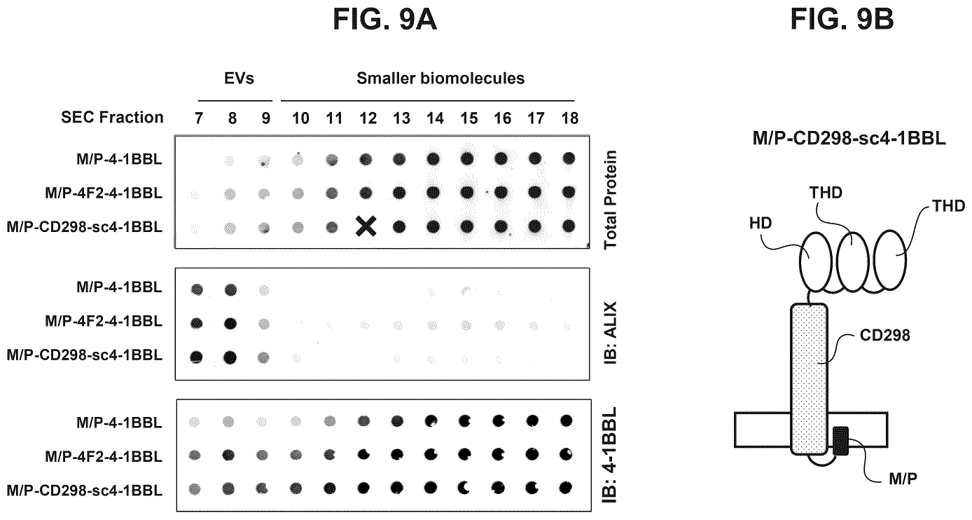

FIG. 9 shows an example of results of extracellular vesicle (EV) purification from cellular conditioned medium using size exclusion chromatography (SEC) and characterization of SEC fractions. Referring to FIG. 9A, dot blot analysis was performed on fractions collected during SEC purification of extracellular vesicles engineered to display various fusion polypeptide embodiments. For example, Flp-In 293 cells engineered to express an embodiment of a fusion polypeptide presented herein (e.g., M/P-4-1BBL, or M/P-4F2-4-1BBL, or M/P-CD298-sc4-1BBL) were incubated in serum-free conditioned media within a culture vessel known by one of ordinary skill in the art. The serum-free conditioned media from Flp-In 293 cells expressing a fusion polypeptide embodiment was harvested and filtered through a 0.45 μm PES filter. The filtered media was concentrated and buffer-exchanged to Dulbecco's phosphate buffered saline (DPBS) using an Amicon Ultra-15 Centrifugal Filter with a 10 kDa molecular weight cutoff. Extracellular vesicles were separated and purified from smaller biomolecules using qEVoriginal/35 nm size exclusion chromatography (SEC) columns (Izon Science) essentially according to the manufacturer's instructions herein the contents of which are incorporated by reference in their entirety (support.izon.com/qev-columns#user-guides). Following the SEC column void volume (fractions 1-6), fractions 7-18 were collected. Fractions 7-18 were blotted onto PVDF-FL membrane under vacuum suction using a 96-well Bio-Dot Microfiltration Apparatus (Bio-Rad), as a simplified Western Blot procedure, and probed with antibodies to an endogenous extracellular vesicle protein (ALIX) and a POI of the fusion polypeptide (e.g., 4-1BBL), followed by appropriate fluorescent secondary antibodies, or stained for total protein using Li-Cor Revert 700 total protein stain. The blots were imaged and analyzed using a Li-Cor Odyssey fluorescent imager. Results show SEC fractions 7, 8, and 9 with the highest level of endogenous extracellular vesicle protein ALIX, indicating that fractions 7, 8 and 9 comprise extracellular vesicles. Purified extracellular fractions 7, 8, and 9 also showed staining for 4-1BBL as determined by dot blot analysis, indicating that fractions 7, 8 and 9 comprise extracellular vesicles displaying the fusion polypeptide embodiments as described herein.

FIG. 9B shows a Type II membrane fusion polypeptide displayed on the surface of an engineered extracellular vesicle comprising a CD298 vesicle targeting domain, a multi-effector domain, and a plurality of linkers, the C-Terminus being on the exterior of the exosome membrane while the N-Terminus being on the interior. A preferred embodiment of a fusion polypeptide as shown in FIG. 9B may be M/P-CD298-sc4-1BBL.

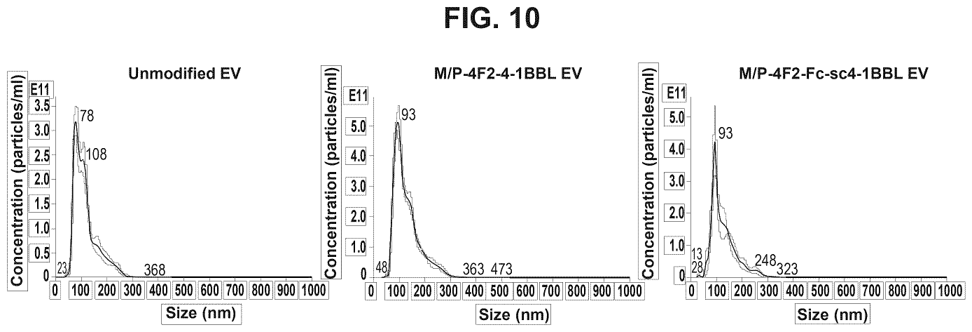

FIG. 10 shows size versus concentration histograms of purified extracellular vesicles from Flp-In 293 cells (Unmodified EV) or purified extracellular vesicles from Flp-In 293 cells displaying two exemplary embodiments of fusion polypeptides (M/P-4F2-4-1BBL and M/P-4F2-Fc-sc4-1BBL) using Nanoparticle Tracking Analysis (NTA, NanoSight, Malvern Panalytical). In this example, purified unmodified extracellular vesicles comprised a size distribution between 20 to 500 nm in diameter. In this example, purified extracellular vesicles displaying M/P-4F2-4-1BBL fusion polypeptide comprised a size distribution between 20 to 500 nm in diameter. In this example, purified extracellular vesicles displaying M/P-4F2-Fc-sc4-1BBL fusion polypeptide comprised a size distribution between 20 to 500 nm in diameter. It is understood that the term “particles” and “vesicles” can be used interchangeably herein.

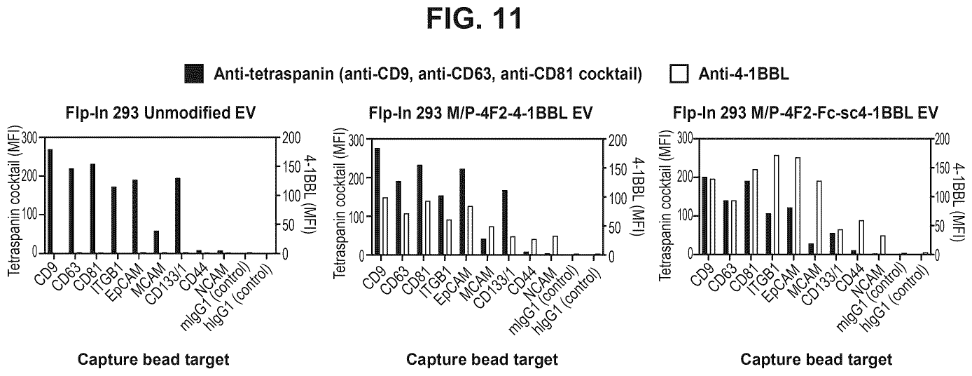

FIG. 11 shows flow cytometry analysis results in Mean Fluorescent Intensity (MFI) of extracellular vesicle epitopes on unmodified extracellular vesicles and epitopes on extracellular vesicles displaying fusion polypeptide embodiments using antibody-conjugated capture beads. The graphs show an analysis of surface epitopes on extracellular vesicles (EV) purified from unmodified Flp-In 293 cells. The graphs show an analysis of surface epitopes on extracellular vesicles (EV) purified from Flp-In 293 cells engineered to display the M/P-4F2-4-1BBL fusion polypeptide. The graphs show an analysis of surface epitopes on extracellular vesicles (EV) purified from Flp-In 293 cells engineered to display the M/P-4F2-Fc-sc4-1BBL fusion polypeptide. Flow cytometry was performed using the MACSPlex Exosome Kit (Miltenyi Biotec) essentially according to the manufacturer's instructions the contents of which are incorporated herein by reference in its entirety (miltenyibiotec.com/upload/assets/IM0015463.PDF). A cocktail of the three fluorescent MACSPlex Exosome Detection Reagents (anti-CD9, anti-CD63, and anti-CD81 for a broad exosome staining) with the addition of a fluorescent anti-4-1BBL antibody in a non-overlapping channel were used. Samples were analyzed with a MACSQuant® flow cytometer (Miltenyi Biotec). Results show that purified extracellular vesicles retained markers for endogenous extracellular vesicle surface proteins, for example endogenous exosome surface proteins (e.g., CD9, CD63, and CD81, etc.). Results show that purified unmodified extracellular vesicles retained markers for endogenous extracellular vesicle surface proteins, for example endogenous exosome surface proteins. Results show that purified extracellular vesicles engineered to display a fusion polypeptide presented herein retained markers for endogenous extracellular vesicle surface proteins, for example endogenous exosome surface proteins, for example tetraspanins. Results show that purified extracellular vesicles engineered to display the fusion polypeptide M/P-4F2-4-1BBL presented herein retained markers for endogenous extracellular vesicle surface proteins, for example endogenous exosome surface proteins (e.g., tetraspanins). Results show that purified extracellular vesicles engineered to display the fusion polypeptide M/P-4F2-Fc-sc4-1BBL presented herein retained markers for endogenous extracellular vesicle surface proteins, for example endogenous exosome surface proteins (e.g., tetraspanins). Unmodified extracellular vesicles (i.e., extracellular vesicles purified from Flp-In 293 cells) and extracellular vesicles modified (i.e., engineered) to display fusion polypeptides (i.e., extracellular vesicles purified from Flp-In 293 cells engineered to display M/P-4F2-4-1BBL or M/P-4F2-Fc-sc4-1BBL)) displayed endogenous extracellular vesicle epitopes, for example CD9, CD63, CD81, ITGB1, EpCAM, MCAM, CD133/1, CD44, and NCAM (MFI of tetraspanin cocktail). Unmodified extracellular vesicles and extracellular vesicles modified to display fusion polypeptides did not bind to capture beads conjugated with isotype control antibodies mIgG1 and hIgG. Extracellular vesicles modified to display fusion polypeptides demonstrated display of the fusion polypeptide. Extracellular vesicles modified to display fusion polypeptide M/P-4F2-4-1BBL demonstrated correlation of display of the fusion polypeptide as shown in increased MFI for 4-1BBL detection with endogenous extracellular markers. Extracellular vesicles modified to display fusion polypeptide M/P-4F2-Fc-sc4-1BBL demonstrated correlation of display of the fusion polypeptide as shown in increased MFI for 4-1BBL detection with endogenous extracellular markers. FIG. 11 results show that a fusion polypeptide as presented herein is displayed on an engineered extracellular vesicle.

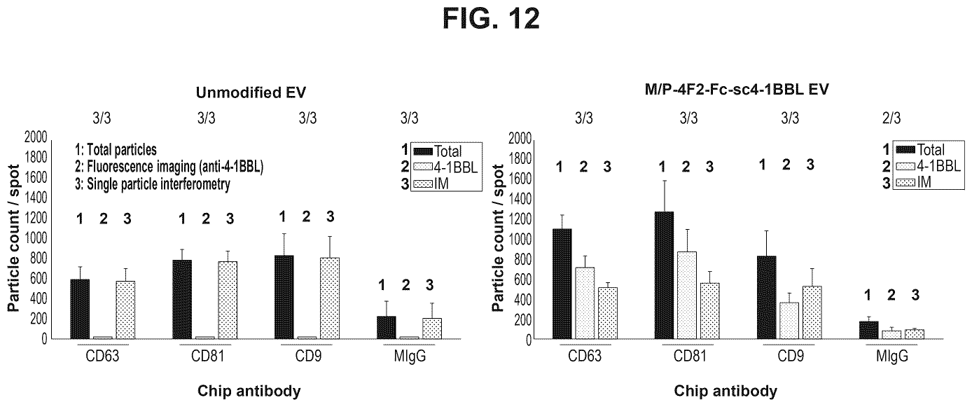

FIG. 12 Shows quantification of Exo View (Unchained Labs) chip-bound EVs by single particle interferometry and immunofluorescence. The Si/SiO₂ double layer chip is tiled with a microarray of individual functionalized antibody spots for EV tetraspanins (CD81, CD63, or CD9) or mIgG as an isotype control for non-specific binding. After an incubation step, each capture spot had bound EVs, which express the corresponding marker. The EVs were counterstained with a fluorescently labeled antibody to 4-1BBL. Total particles (1) is the combination of the overlapping sets of fluorescent particles (2) and particles detected by interferometry measuring 50-200 nm in diameter (3). FIG. 12 shows the fusion polypeptide M/P-4F2-Fc-sc4-1BBL is expressed on individual EVs that express tetraspanins CD63, CD81 or CD9.

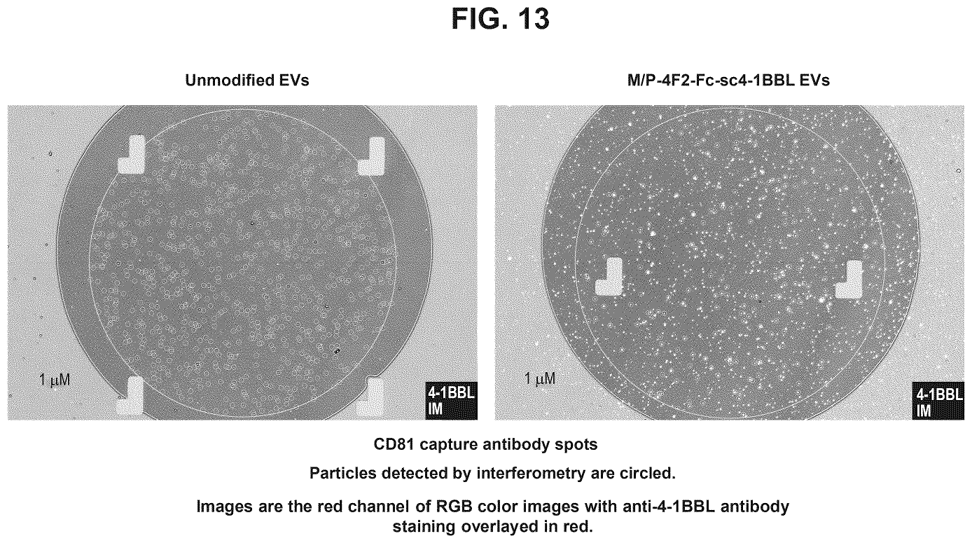

FIG. 13 shows the monochromatic red channel of overlayed photographic and fluorescent images of EVs bound to CD81 antibody functionalized spots on an Exo View (Unchained Labs) chip counterstained with fluorescently labeled antibody to 4-1BBL in red. 50-200 nm diameter particles identified by interferometry are circled. 4-1BBL immunofluorescence is shown as bright spots in the overlayed image. FIG. 13 shows that the fusion polypeptide M/P-4F2-Fc-sc4-1BBL is expressed on CD81 positive vesicles that can be detected by single particle interferometry.

### Example 6. Purification and Analysis of Engineered Extracellular Vesicles Displayed with GITR Agonist Fusion Polypeptide Embodiments

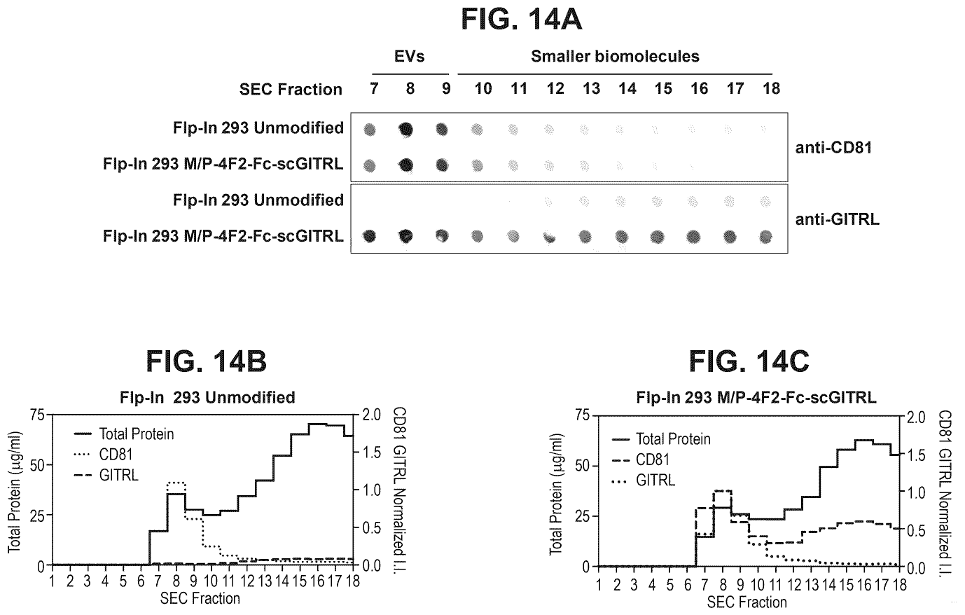

FIG. 14 shows an example of results of extracellular vesicle (EV) purification from cellular conditioned medium using size exclusion chromatography (SEC) and characterization of SEC fractions for total protein, CD81, and GITRL. Referring to FIG. 14A, dot blot analysis was performed on fractions collected during SEC purification of unmodified extracellular vesicles (i.e., extracellular vesicles purified from Flp-In 293 cells) or extracellular vesicles engineered to display a fusion polypeptide embodiment, for example fusion polypeptide embodiment M/P-4F2-Fc-scGITRL. Unmodified Flp-In 293 or Flp-In 293 cells engineered to express an embodiment of a GITRL agonist fusion polypeptide presented herein, e.g., M/P-4F2-Fc-scGITRL, were grown in serum-free conditioned media within a culture vessel known by one of ordinary skill in the art. The serum-free conditioned media from unmodified Flp-In HEK 293 cells, or Flp-In HEK 293 cells expressing the M/P-4F2-Fc-scGITRL fusion polypeptide, was harvested and filtered through a 0.45 μm PES filter. The filtered media was concentrated and buffer-exchanged to Dulbecco's phosphate buffered saline (DPBS) using an Amicon Ultra-15 Centrifugal Filter with a 10 kDa molecular weight cutoff. Extracellular vesicles were separated and purified from smaller biomolecules using qEVoriginal/35 nm size exclusion chromatography (SEC) columns (Izon Science) essentially according to the manufacturer's instructions the contents of which are incorporated herein by reference in their entirety (support.izon.com/qev-columns #user-guides). Following the SEC column void volume (fractions 1-6), fractions 7-18 were collected. Fractions 7-18 were blotted onto PVDF-FL membrane under vacuum suction using a 96-well Bio-Dot Microfiltration Apparatus (Bio-Rad) and probed with antibodies to an endogenous extracellular vesicle protein (for example, anti-CD81 antibodies) and a component of the GITRL agonist fusion polypeptide (for example anti-GITRL antibodies), followed by appropriate fluorescent secondary antibodies. The blots were imaged and analyzed using a Li-Cor Odyssey fluorescent imager. Results show SEC fractions 7, 8, and 9 from unmodified Flp-In extracellular vesicles and SEC fractions 7, 8, and 9 from extracellular vesicles engineered to display the M/P-4F2-Fc-scGITRL fusion polypeptide display endogenous extracellular vesicle protein CD81, indicating that fractions 7, 8 and 9 comprise extracellular vesicles. Results show SEC fractions 7, 8, and 9 from purified extracellular vesicles engineered to display the M/P-4F2-Fc-scGITRL display the said fusion polypeptide, indicating that fractions 7, 8 and 9 comprise extracellular vesicles displaying fusion polypeptide embodiments as described herein. Results show SEC fractions 7, 8, and 9 from purified nonengineered Flp-In extracellular vesicles did not display the M/P-4F2-Fc-scGITRL fusion polypeptide. FIG. 14B shows plots of total protein and background subtracted integrated intensity of SEC fractions one through eighteen collected from purification of unmodified extracellular vesicles and purification of extracellular vesicles engineered to display a preferred fusion polypeptide embodiment, for example M/P-4F2-Fc-scGITRL. Referring to both graphs in FIG. 14B and FIG. 14C, plotted on the left-Y-axis is the total protein concentration of each fraction one through eighteen measured by using fluorometric Qubit protein assays and a Qubit fluorometer (ThermoFisher Scientific), and plotted on the right-Y-axis is the background subtracted integrated intensity (I.I.) of SEC fraction one through eighteen spots determined using Li-Cor Odyssey software and normalized to the I.I. of fraction 8. CD81 is an endogenous exosome marker and is present in fractions containing extracellular vesicles, for example fractions 7-12 in FIG. 14B and fractions 7-13 in FIG. 14C. GITRL intensity normalized to the I.I. of fraction 8 reveals that the M/P-4F2-Fc-scGITRL fusion polypeptide corresponds to the exosome marker CD81, indicating that the fusion polypeptide 4F2-Fc-scGITRL is associated with extracellular vesicles.

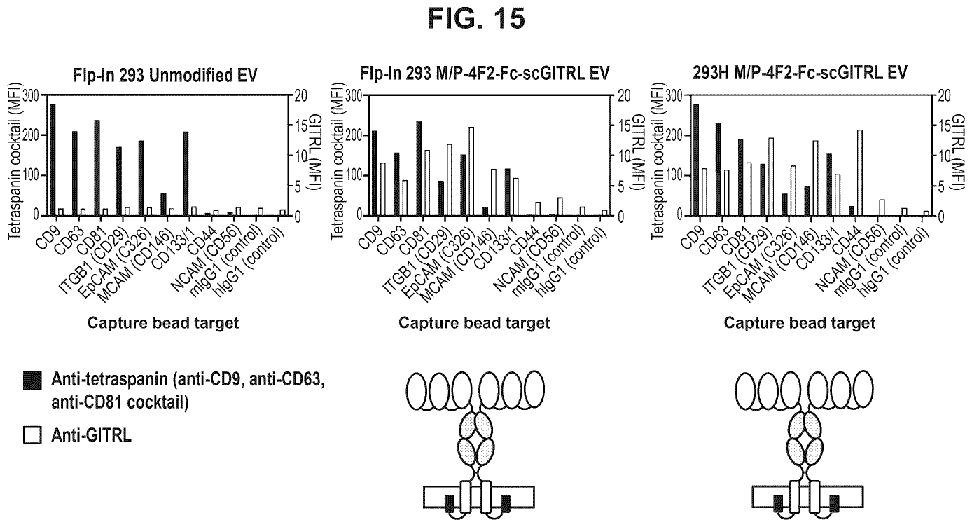

FIG. 15 shows flow cytometry analysis results in Mean Fluorescent Intensity (MFI) of extracellular vesicle epitopes on unmodified extracellular vesicles and epitopes on extracellular vesicles displaying fusion polypeptide embodiments using antibody-conjugated capture beads. The graphs show an analysis of surface epitopes on extracellular vesicles purified from unmodified Flp-In 293 cells. The graphs show an analysis of surface epitopes on extracellular vesicles purified from Flp-In 293 cells engineered to display the M/P-4F2-Fc-scGITRL fusion polypeptide embodiment. The graphs show an analysis of surface epitopes on extracellular vesicles purified from 293H cells engineered to display the M/P-4F2-Fc-scGITRL fusion polypeptide embodiment. Flow cytometry was performed using the MACSPlex Exosome Kit (Miltenyi Biotec) essentially according to the manufacturer's instructions the contents of which are incorporated herein by reference in its entirety (miltenyibiotec.com/upload/assets/IM0015463.PDF). A cocktail of the three fluorescent MACSPlex Exosome Detection Reagents (anti-CD9, anti-CD63, and anti-CD81 for a broad exosome staining) with the addition of a fluorescent anti-GITRL antibody in a non-overlapping channel were used. Samples were analyzed with a MACSQuant® flow cytometer (Miltenyi Biotec). Results show that purified extracellular vesicles retained markers for endogenous extracellular vesicle surface proteins, for example endogenous exosome surface proteins (e.g., CD9, CD63, and CD81, etc.). Results show that purified unmodified extracellular vesicles retained markers for endogenous extracellular vesicle surface proteins, for example endogenous exosome surface proteins. Results show that purified extracellular vesicles engineered to display a fusion polypeptide presented herein retained markers for endogenous extracellular vesicle surface proteins, for example endogenous exosome surface proteins, for example tetraspanins. Results show that purified extracellular vesicles from Flp-In 293 cells engineered to display the fusion polypeptide M/P-4F2-Fc-scGITRL as presented herein retained markers for endogenous extracellular vesicle surface proteins, for example endogenous exosome surface proteins, e.g., tetraspanins. Results show that purified extracellular vesicles from 293H cells engineered to display the fusion polypeptide M/P-4F2-Fc-scGITRL as presented herein retained markers for endogenous extracellular vesicle surface proteins, for example endogenous exosome surface proteins, e.g., tetraspanins. Extracellular vesicles purified from unmodified Flp-In 293 cells (Flp-In 293 unmodified) and extracellular vesicles from Flp-In 293 cells engineered to display the fusion polypeptide embodiment M/P-4F2-Fc-scGITRL and suspension 293H cells engineered to display the fusion polypeptide embodiment M/P-4F2-Fc-scGITRL, displayed endogenous extracellular vesicle epitopes, for example CD9, CD63, CD81, ITGB1 (CD29), EpCAM (CD326), MCAM (CD146), CD133/1, CD44, and NCAM (CD56) (MFI of tetraspanin cocktail). Unmodified extracellular vesicles did not display epitopes that are not associated with extracellular vesicles, for example mIgG1 and hIgG. Extracellular vesicles engineered to display the fusion polypeptide embodiment M/P-4F2-Fc-scGITRL purified from Flp-In 293 or 293H cells presented herein were positive for epitopes mIgG1 and hIgG indicating that the Fc linker is detected in the flow cytometry and correlates with exosome markers. These results show that extracellular vesicles engineered to display fusion polypeptides demonstrated display of said fusion polypeptide. Extracellular vesicles engineered to display fusion polypeptide M/P-4F2-Fc-scGITRL demonstrated display of the fusion polypeptide as shown in increased MFI for GITRL detection. Extracellular vesicles engineered to display fusion polypeptide M/P-4F2-Fc-scGITRL demonstrated display of the fusion polypeptide as shown in increased MFI for GITRL detection. These results demonstrate that extracellular vesicles purified from Flp-In 293 or 293H cells engineered to express an agonistic fusion polypeptide as presented herein in all embodiments comprise endogenous extracellular vesicle proteins as known by one familiar in the art and further comprise said engineered fusion polypeptide.

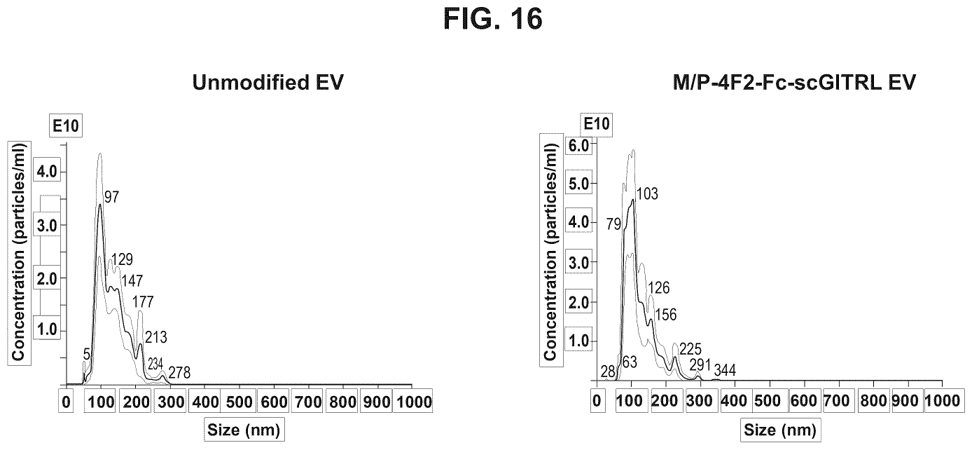

FIG. 16 shows size versus concentration histograms of purified extracellular vesicles from 293H cells (Unmodified EV) or purified extracellular vesicles from 293H cells displaying the exemplary embodiments of the fusion polypeptide M/P-4F2-Fc-scGITRL using Nanoparticle Tracking Analysis (NTA, NanoSight, Malvern Panalytical). In this example, purified unmodified extracellular vesicles comprised a size distribution between 20 to 500 nm in diameter. In this example, purified extracellular vesicles displaying M/P-4F2-Fc-scGITRL fusion polypeptide comprised a size distribution between 20 to 500 nm in diameter. It is understood that the term “particles” and “vesicles” can be used interchangeably herein.

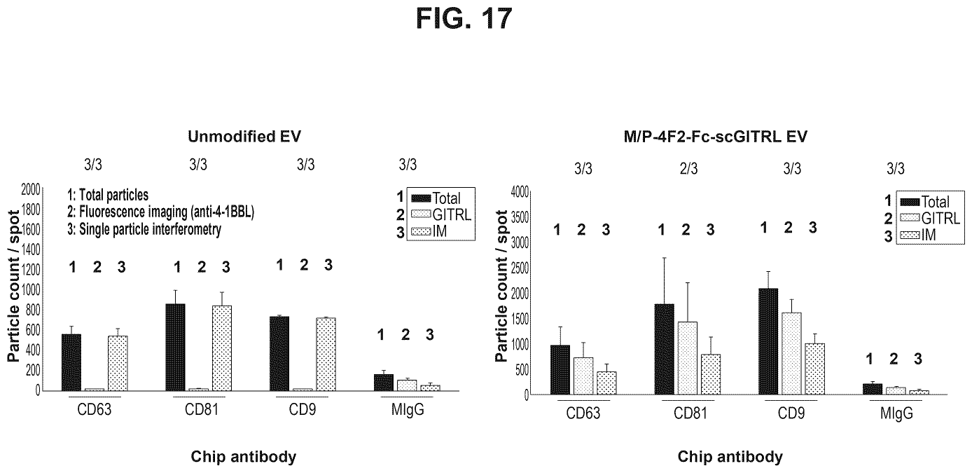

FIG. 17 Shows quantification of ExoView (Unchained Labs) chip-bound EVs by single particle interferometry and immunofluorescence. The Si/SiO₂ double layer chip is tiled with a microarray of individual functionalized antibody spots for EV tetraspanins (CD81, CD63, or CD9) or mIgG as an isotype control for non-specific binding. After an incubation step, each capture spot had bound EVs, which express the corresponding marker. The EVs were counterstained with a fluorescently labeled antibody to GITRL. Total particles (1) is the combination of the overlapping sets of fluorescent particles (2) and particles detected by interferometry measuring 50-200 nm in diameter (3). FIG. 17 shows the fusion polypeptide M/P-4F2-Fc-scGITRL is expressed on individual EVs that express tetraspanins CD63, CD81 or CD9.

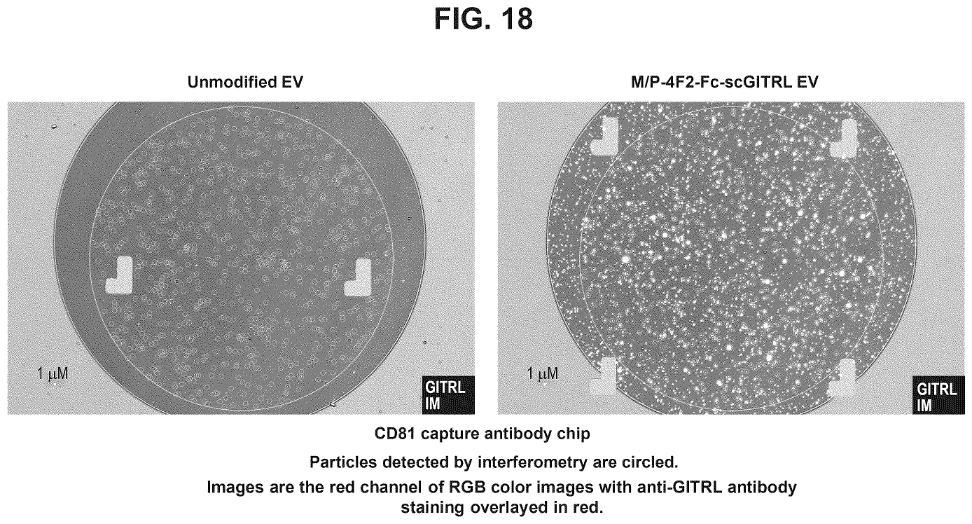

FIG. 18 shows the monochromatic red channel of overlayed photographic and fluorescent images of EVs bound to CD81 antibody functionalized spots on an Exo View (Unchained Labs) chip counterstained with fluorescently labeled antibody to GITRL in red. 50-200 nm diameter particles identified by interferometry are circled. 4-1BBL immunofluorescence is shown as bright spots in the overlayed image. FIG. 18 shows that the fusion polypeptide M/P-4F2-Fc-scGITRL is expressed on CD81 positive vesicles that can be detected by single particle interferometry.

### Example 7. Purification and Analysis of Engineered Extracellular Vesicles Displayed with OX40 Agonist Fusion Polypeptide Embodiments

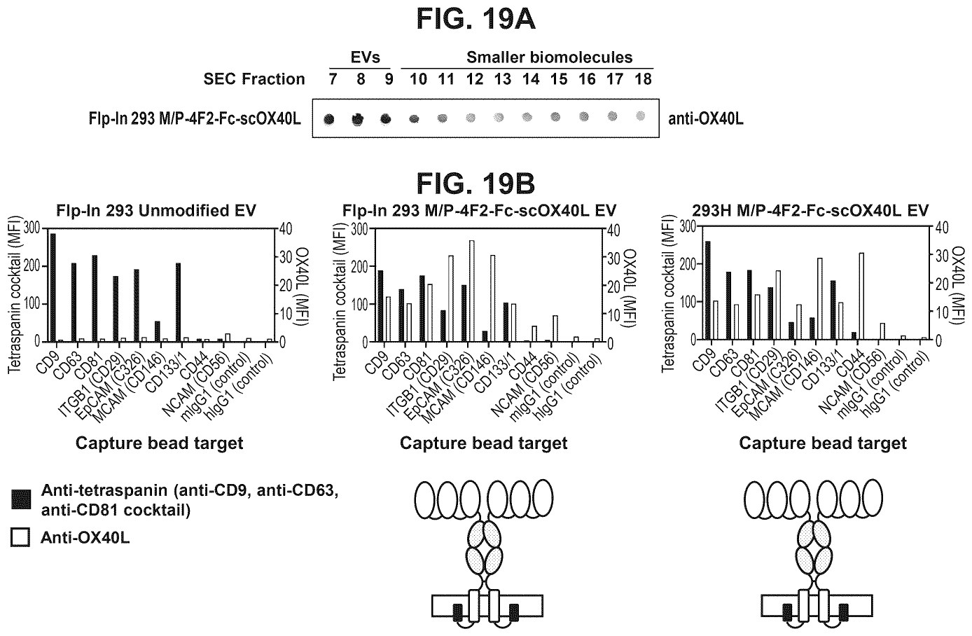

FIG. 19 shows an example of results of extracellular vesicle purification from cellular conditioned medium using size exclusion chromatography (SEC) and characterization of SEC fractions for surface proteins and OX40L. Referring to FIG. 19A, dot blot analysis was performed on fractions collected during SEC purification of extracellular vesicles engineered to display a fusion polypeptide embodiment, for example fusion polypeptide embodiment M/P-4F2-Fc-scOX40L. For example, Flp-In 293 cells engineered to express an embodiment of a OX40L fusion polypeptide presented herein, for example M/P-4F2-Fc-scOX40L, were grown in serum-free conditioned media within a culture vessel known by one of ordinary skill in the art. The serum-free conditioned media from Flp-In 293 cells engineered to the fusion polypeptide embodiment M/P-4F2-Fc-scOX40L was harvested and filtered through a 0.45 μm PES filter. The filtered media was concentrated and buffer-exchanged to Dulbecco's phosphate buffered saline (DPBS) using an Amicon Ultra-15 Centrifugal Filter with a 100 kDa molecular weight cutoff. Extracellular vesicles were separated and purified from smaller biomolecules using qEVoriginal/35 nm size exclusion chromatography (SEC) columns (Izon Science) essentially according to the manufacturer's instructions the contents of which are incorporated herein by reference in their entirety (support.izon.com/qev-columns #user-guides). Following the SEC column void volume (fractions 1-6), fractions 7-18 were collected. Fractions 7-18 were blotted onto PVDF-FL membrane under vacuum suction using a 96-well Bio-Dot Microfiltration Apparatus (Bio-Rad) and probed with antibodies to a domain of the fusion polypeptide (for example anti-OX4OL antibodies), followed by detection fluorescent secondary antibodies. The blots were imaged and analyzed using a Li-Cor Odyssey fluorescent imager. Results show SEC fractions 7, 8, and 9 with the highest level of expression of the fusion polypeptide, indicating that fractions 7, 8 and 9 comprise extracellular vesicles displaying fusion polypeptide embodiments as described herein, for example M/P-4F2-Fc-scOX40L.

FIG. 19B shows flow cytometry analysis results in Mean Fluorescent Intensity (MFI) of extracellular vesicle epitopes on unmodified extracellular vesicles and epitopes on extracellular vesicles displaying fusion polypeptide embodiments using antibody-conjugated capture beads. The graphs show an analysis of surface epitopes on extracellular vesicles purified from unmodified Flp-In 293 cells. The graphs show an analysis of surface epitopes on extracellular vesicles purified from Flp-In 293 cells engineered to display the M/P-4F2-Fc-sc OX40L fusion polypeptide embodiment. The graphs show an analysis of surface epitopes on extracellular vesicles purified from 293H cells engineered to display the M/P-4F2-Fc-sc OX40L fusion polypeptide embodiment. Flow cytometry was performed using the MACSPlex Exosome Kit (Miltenyi Biotec) essentially according to the manufacturer's instructions the contents of which is incorporated herein by reference in its entirety (miltenyibiotec.com/upload/assets/IM0015463.PDF). A cocktail of the three fluorescent MACSPlex Exosome Detection Reagents (anti-CD9, anti-CD63, and anti-CD81 for a broad exosome staining) with the addition of a fluorescent anti-OX40L antibody in a non-overlapping channel were used. Samples were analyzed with a MACSQuant® flow cytometer (Miltenyi Biotec). Results show that purified extracellular vesicles retained markers for endogenous extracellular vesicle surface proteins, for example endogenous exosome surface proteins (e.g., CD9, CD63, and CD81, etc.). Results show that purified unmodified extracellular vesicles retained markers for endogenous extracellular vesicle surface proteins, for example endogenous exosome surface proteins. Results show that purified extracellular vesicles engineered to display a fusion polypeptide presented herein retained markers for endogenous extracellular vesicle surface proteins, for example endogenous exosome surface proteins, for example tetraspanins. Results show that purified extracellular vesicles from Flp-In 293 cells engineered to display the fusion polypeptide M/P-4F2-Fc-sc OX40L as presented herein retained markers for endogenous extracellular vesicle surface proteins, for example endogenous exosome surface proteins, e.g., tetraspanins. Results show that purified extracellular vesicles from 293H cells engineered to display the fusion polypeptide M/P-4F2-Fc-sc OX40L as presented herein retained markers for endogenous extracellular vesicle surface proteins, for example endogenous exosome surface proteins, e.g., tetraspanins. Extracellular vesicles purified from unmodified Flp-In 293 cells (Flp-In 293 unmodified) and extracellular vesicles from Flp-In 293 cells engineered to display the fusion polypeptide embodiment M/P-4F2-Fc-sc OX40L and suspension 293H cells engineered to display the fusion polypeptide embodiment M/P-4F2-Fc-sc OX40L, displayed endogenous extracellular vesicle epitopes, for example CD9, CD63, CD81, ITGB1 (CD29), EpCAM (CD326), MCAM (CD146), CD133/1, CD44, and NCAM (CD56) (MFI of tetraspanin cocktail). Unmodified extracellular vesicles did not display epitopes that are not associated with extracellular vesicles, for example mIgG1 and hIgG. Extracellular vesicles engineered to display the fusion polypeptide embodiment M/P-4F2-Fc-sc OX40L purified from Flp-In 293 or 293H cells presented herein were positive for epitopes mIgG1 and hIgG indicating that the Fc linker is detected in the flow cytometry and correlates with exosome markers. These results show that extracellular vesicles engineered to display fusion polypeptides demonstrated display of said fusion polypeptide. Extracellular vesicles engineered to display fusion polypeptide M/P-4F2-Fc-sc OX40L demonstrated display of the fusion polypeptide as shown in increased MFI for OX40L detection. Extracellular vesicles engineered to display fusion polypeptide M/P-4F2-Fc-sc OX40L demonstrated display of the fusion polypeptide as shown in increased MFI for OX40L detection. These results demonstrate that extracellular vesicles purified from Flp-In 293 or 293H cells engineered to express an agonistic fusion polypeptide as presented herein in all embodiments comprise endogenous extracellular vesicle proteins as known by one familiar in the art and further comprise said engineered fusion polypeptide.

### Example 8. 4-1BB Agonism

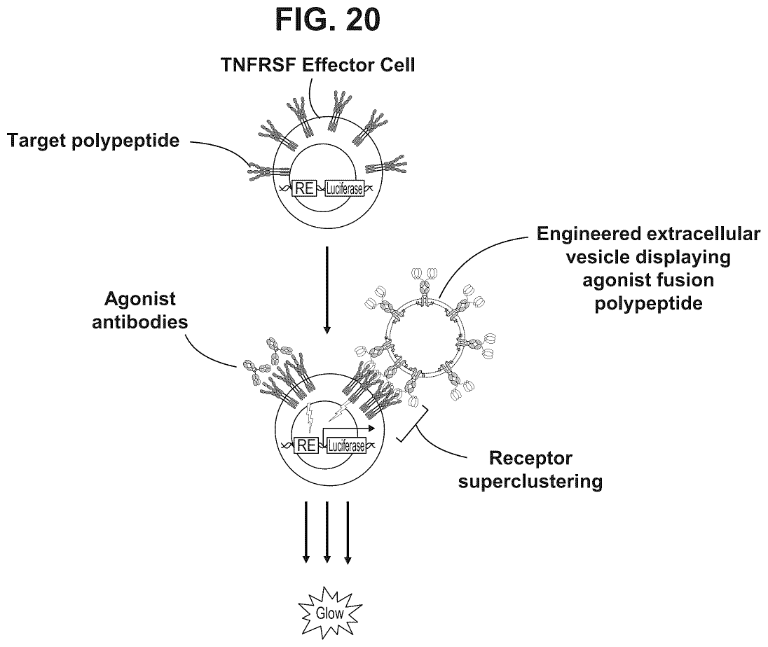

FIG. 20 shows an assay system comprising of a genetically engineered Jurkat T cell line that expresses a target protein and a luciferase reporter driven by a response element that responds to stimulation of the target protein receptor. The assay system is a genetically engineered Jurkat T cell line that expresses human TNF receptor superfamily member (TNFRSF) (e.g., 4-1BB, GITR, or OX40 or the like) and a luciferase reporter driven by a response element that can respond to ligand/agonist antibody stimulation of the TNFRSF receptor. Various embodiments of the assay system are manufactured and distributed by Promega Corporation, Madison, Wisconsin, United Stated.

In some embodiments presented herein the commercially available Promega 4-1BB (CD137/TNFRSF9) bioassay kit was used to evaluate agonist effects of engineered extracellular vesicles displaying fusion polypeptides of various embodiment presented herein comprising 4-1BBL protein of interest or fragments thereof. 4-1BB is a member of the tumor necrosis factor receptor superfamily, is an inducible co-stimulatory receptor expressed on various immune cell populations, for example T cells, natural killer (NK) cells and innate immune cells. Cell surface displayed 4-1BB interacts with 4-1BB ligand (4-1BBL) and induces subsequent cell proliferation and production of interferon gamma (IFNγ) and interleukin-2 (IL-2), particularly in T and NK cells. Promega's 4-1BB Bioassay is a biologically relevant, mechanism of action (MOA) based assay that can be used to measure the potency and stability of agonists that can bind to and activate 4-1BB target receptor. 4-1BB agonist bioassays were performed using the Promega 4-1BB Bioassay kit essentially according to the manufacturer's instructions, the contents of which is incorporated herein by reference in its entirety (promega.com/products/reporter-bioassays/immune-checkpoint-bioassays/4-1bb-bioassay/?catNum-JA2351). In some experiments, the Promega 41BB Bioassay was modified to evaluate agonism by extracellular vesicles displaying fusion polypeptide embodiment.

In some embodiments presented herein the commercially available Promega GITR (CD357/TNFRSF18) bioassay kit was used to evaluate agonist effects of engineered extracellular vesicles displaying fusion polypeptides of various embodiment presented herein comprising GITRL protein of interest or fragments thereof. The GITR Bioassays are bioluminescent cell-based assays used to measure the potency and stability of ligands or agonist antibodies that bind and activate GITR target receptor. GITR (CD357/TNFRSF18), a member of the tumor necrosis factor (TNF) receptor superfamily, is a costimulatory receptor widely expressed on most immune cells and further upregulated on activated T cells. When engaged with a GITR agonist, for example GITR ligand (GITRL) on the cell surface, GITR enhances subsequent T cell expansion and cytokine production including interleukin-2 (IL-2) and IL-9. The GITR Bioassay reflects the mechanism of action (MOA) of biologics designed to activate GITR. GITR agonist bioassays were performed using the Promega GITR Bioassay kit essentially according to the manufacturer's instructions, the contents of which is incorporated herein by reference in its entirety (promega.com/products/reporter-bioassays/immune-checkpoint-bioassays/gitr-bioassays/?catNum=JA2291). In some experiments, the Promega GITR Bioassay was modified to evaluate agonism by extracellular vesicles displaying fusion polypeptide embodiment.

In some embodiments presented herein the commercially available Promega OX40 (CD134/TNFRSF4) bioassay kit was used to evaluate agonist effects of engineered extracellular vesicles displaying fusion polypeptides of various embodiment presented herein comprising OX40L protein of interest or fragments thereof. The OX40 Bioassay is a bioluminescent cell-based assay that measures the potency and stability of ligands or agonist antibodies that can bind and activate OX40 target receptor. OX40 (CD134/TNFRSF4), a member of the tumor necrosis factor (TNF) receptor superfamily, is a costimulatory receptor expressed primarily on activated T cells, and on neutrophils and natural killer (NK) cells to a lesser extent. When present on the cell surface, OX40 interacts with OX40 ligand (OX40L) and induces subsequent cell proliferation, survival, and production of cytokines, particularly in T cells. Promega's OX40 Bioassay reflects the mechanism of action (MOA) of biologics designed to activate OX40 target receptor following the addition of OX40 ligand or OX40 agonists. OX40 agonist bioassays were performed using the Promega OX40 Bioassay kit essentially according to the manufacturer's instructions, the contents of which is incorporated herein by reference in its entirety (promega.com/products/reporter-bioassays/immune-checkpoint-bioassays/ox40-bioassays/?catNum=JA2191). In some experiments, the Promega OX40 Bioassay was modified to evaluate agonism by extracellular vesicles displaying fusion polypeptide embodiment.

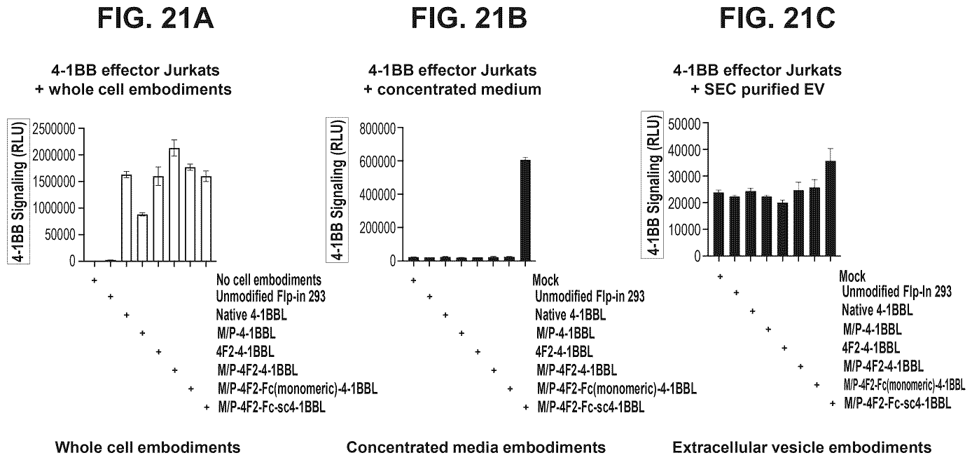

FIG. 21A shows relative 4-1BB agonism as indicated in relative light units (RLU) of Jurkat effector cells induced by cells displaying various fusion polypeptide embodiments compared to control unmodified Flp-In 293 cells. Cells expressing the presented fusion polypeptide embodiments were co-cultured with Promega Bioassay 4-1BB effector Jurkat cells for 6 hours. The Jurkat cells were then treated with a luciferase substrate and enzymatic activity was measured using a plate fluorometer. 4-1BB agonist signaling was not observed in Jurkat cells alone, i.e., when no other cell was added to the bioassay. 4-1BB agonist signaling was not observed in Jurkat cells when unmodified Flp-In 293 cells were added to the bioassay. 4-1BB agonist signaling was observed in Jurkat cells when cells displaying native full length 4-1BBL or a fusion polypeptide embodiment comprising a 4-1BBL fragment was used in the bioassay. For example, cells displaying a M/P-4-1BBL, 4F2-4-1BBL, M/P-4F2-4-1BBL, MP-4F2-Fc (monomeric)-4-1BBL, M/P-4F2-Fc-sc4-1BBL fusion polypeptide or native 4-1BBL exhibited 4-1BB signaling (4-1BB agonism) as indicated in RLU 4-1BB.

FIG. 21B shows relative 4-1BB agonism as indicated in relative light units (RLU) of Jurkat effector cells induced by filtered media collected after the growth of unmodified Flp-In cells, and filtered media collected after the growth of cells engineered to display native full length 4-1BBL (native 4-1BBL), and filtered media collected after the growth of cells displaying various embodiments of fusion polypeptides (e.g., M/P-4-1BBL, 4F2-4-1BBL, M/P-4F2-4-1BBL, M/P-4F2-Fc-sc4-1BBL). Referring to FIG. 21B, conditioned media from cells genetically modified to express a fusion polypeptide embodiment, or media from Flp-In293 cells expressing native full length 4-1BBL, or media from unmodified Flp-In 293 cells was harvested and then concentrated using an Amicon Ultra-15 Centrifugal Filter with a 10 kDa molecular weight cutoff. The concentrated media embodiments were then added to 4Promega Bioassay 4-1BB effector Jurkat cells for 6 hours. The Jurkat cells were then treated with a luciferase substrate and enzymatic activity was measured using a plate fluorometer. 4-1BB agonist signaling in Jurkat cells was not observed when no other cells were added to the bioassay (i.e., in Jurkat cells alone) (mock). 4-1BB agonist signaling in Jurkat cells was not observed when concentrated medium derived from unmodified Flp-In 293 cells was bioassayed. 4-1BB agonist signaling in Jurkat cells was not observed when concentrated medium derived from cells displaying native full length 4-1BBL was added to the bioassay. 4-1BB agonist signaling in Jurkat cells was not observed when concentrated medium derived from cells displaying M/P-4-1BBL fusion polypeptide was added to the bioassay. 4-1BB agonist signaling in Jurkat cells was not observed when concentrated medium derived from cells displaying 4F2-4-1BBL fusion polypeptide was added to the bioassay. 4-1BB agonist signaling in Jurkat cells was not observed when concentrated medium derived from cells displaying M/P-4F2-4-1BBL fusion polypeptide was added to the bioassay. 4-1BB agonist signaling in Jurkat cells was not observed when concentrated medium derived from cells displaying MP-4F2-Fc (monomeric)-4-1BBL fusion polypeptide was added to the bioassay. Substantial 4-1BB agonist signaling in Jurkat cells was detected when concentrated medium derived from cells displaying M/P-4F2-Fc-sc4-1BBL fusion polypeptide was added to the bioassay. These results indicate that the concentrated cell culture media from cells engineered to express the M/P-4F2-Fc-sc4-1BBL fusion polypeptide is agonistic to Promega 4-1BB effector Jurkat cells.

FIG. 21C shows relative 4-1BB agonism as indicated in relative light units (RLU) of Jurkat effector cells induced by SEC purified extracellular vesicles displaying various embodiments of fusion polypeptide (e.g., M/P-4-1BBL, 4F2-4-1BBL, M/P-4F2-4-1BBL, M/P-4F2-Fc-sc4-1BBL), or SEC purified unmodified extracellular vesicles from unmodified Flp-In 293 cells, or SEC purified extracellular vesicles displaying native 4-1BBL. Referring to FIG. 21C, SEC purified extracellular vesicles expressing the presented fusion polypeptide embodiments presented herein or SEC purified Flp-In 293 derived extracellular vesicles, or extracellular vesicles displaying the native full length 4-1BL protein were added to Promega Bioassay 4-1BB effector Jurkat cells for 6 hours. The Jurkat cells were then treated with a luciferase substrate and enzymatic activity was measured using a plate fluorometer. RLU, relative fluorescence units. The highest 4-1BB agonist signaling in Jurkat cells was observed when purified extracellular vesicles displaying the M/P-4F2-Fc-sc4-1BBL fusion polypeptide was added to the bioassay. These results empirically demonstrate that M/P-4F2-Fc-sc4-1BBL is a preferred embodiment of a 4-1BBL fusion polypeptide. These results empirically demonstrate that a multi-effector domain is superior to a monomeric 4-1BBL.

4-1BB agonism was detected using genetically engineered Jurkat T cells that expresses human 4-1BB and a luciferase reporter driven by a response element that can respond to 4-1BB agonism (Promega). 4-1BB agonist bioassays were performed using the Promega 4-1BB Bioassay kit essentially according to the manufacturer's instructions, the contents of which is incorporated herein by reference in its entirety (promega.com/products/reporter-bioassays/immune-checkpoint-bioassays/4-1bb-bioassay/?catNum=JA2351). In some experiments, the Promega 4-1BB Bioassay was modified to evaluate agonism by genetically engineered whole cells displaying a fusion polypeptide, or to evaluate agonism by concentrated culture media, to evaluate agonism by extracellular vesicles displaying fusion polypeptides, or unmodified extracellular vesicles purified from cells.

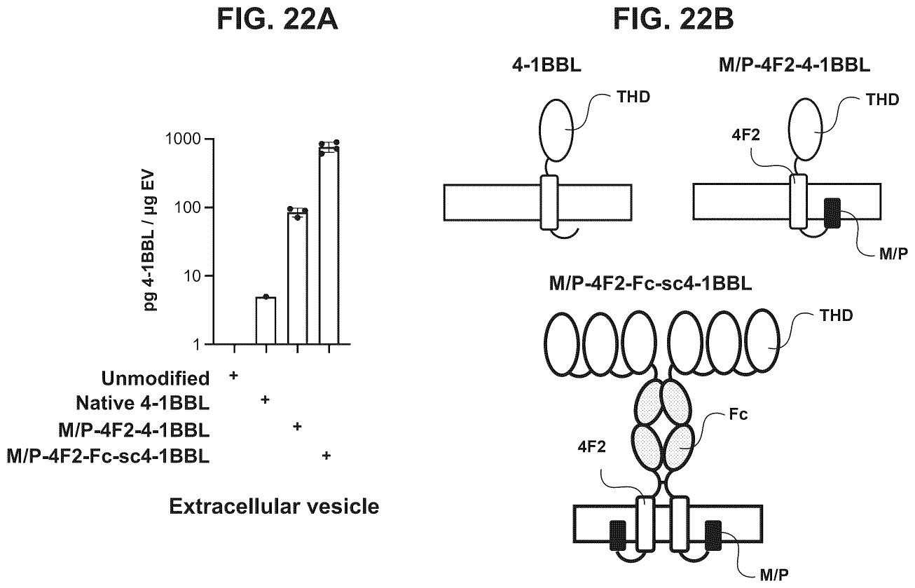

FIG. 22A shows the picogram concentration of 4-1BBL per μg of purified extracellular vesicles from unmodified Flp-In 293 cells, or purified extracellular vesicles displaying native 4-1BBL, or purified extracellular vesicles displaying M/P-4F2-4-1BBL fusion polypeptide embodiment, or purified extracellular vesicles displaying M/P-4F2-Fc-sc4-1BBL fusion polypeptide embodiment. Concentrations of 4-1BBL were determined using a RayBiotech Human 4-1 BB Ligand ELISA Kit essentially according to the manufacturer's instructions, the contents of which are incorporated herein by reference in their entirety (doc.raybiotech.com/pdf/Manual/ELH-41BBL_2021.06.14.pdf). Per μg of purified extracellular vesicles, extracellular vesicles displaying the M/P-4F2-Fc-sc4-1BBL fusion polypeptide embodiment showed substantially higher concentration of 4-1BBL than native full length 4-1BBL or extracellular vesicles displaying the M/P-4F2-1BBL fusion polypeptide embodiment, demonstrating empirically that M/P-4F2-Fc-sc4-1BBL is a preferred embodiment of a 4-1BBL fusion polypeptide. Per μg of purified extracellular vesicles, extracellular vesicles displaying the M/P-4F2-Fc-sc4-1BBL fusion polypeptide showed substantially higher concentration of 4-1BBL than purified extracellular vesicles displaying a M/P-4F2-4-1BBL fusion polypeptide or extracellular vesicles displaying the native 4-1BBL protein. 4-1BBL ELISA results demonstrate that extracellular vesicles purified from unmodified Flp-In 293 do not display 4-1BBL. FIG. 22B shows schematic diagrams of the extracellular vesicle embodiments presented herein FIG. 22A.

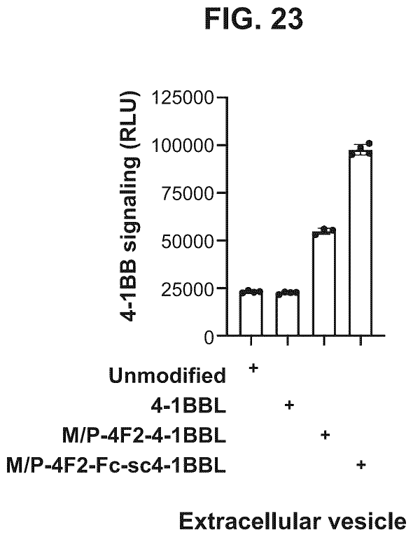

FIG. 23 shows relative 4-1BB agonism as indicated in relative light units (RLU) of Jurkat 4-1BB effector cells induced by SEC purified extracellular vesicles displaying embodiments of fusion polypeptide presented herein (e.g., M/P-4F2-4-1BBL or M/P-4F2-Fc-sc4-1BBL), or SEC purified unmodified extracellular vesicles from Flp-In 293 cells, or SEC purified extracellular vesicles displaying native full length 4-1BBL. SEC purified extracellular vesicles displaying the presented fusion polypeptide embodiment, or SEC purified Flp-In derived extracellular vesicles, or SEC purified extracellular vesicles displaying native full length 4-1BBL were added to Promega Bioassay 4-1BB effector Jurkat cells for 6 hours. The Jurkat cells were then treated with a luciferase substrate and enzymatic activity was measured using a plate fluorometer. RLU, relative fluorescence units. 4-1BB agonist signaling in Jurkat cells was substantially higher when purified extracellular vesicles displaying the M/P-4F2-Fc-sc4-1BBL fusion polypeptide was added to the bioassay when compared to unmodified EVs, EVs displaying a native full length 4-1BBL protein, or EVs displaying fusion polypeptide embodiment M/P-4F2-4-1BBL. 4-1BB agonism was detected using genetically engineered Jurkat T cells that expresses human 4-1BB and a luciferase reporter driven by a response element that can respond to 4-1BB agonism (Promega). 4-1BB agonist bioassays were performed using the Promega 4-1BB Bioassay kit essentially according to the manufacturer's instructions, the contents of which is incorporated herein by reference in its entirety (promega.com/products/reporter-bioassays/immune-checkpoint-bioassays/4-1bb-bioassay/?catNum=JA2351). In some experiments, the Promega 4-1BB Bioassay was modified to evaluate agonism by extracellular vesicles displaying fusion polypeptides, or unmodified extracellular vesicles purified from cells, or extracellular vesicles displaying native fell length 4-1BBL.

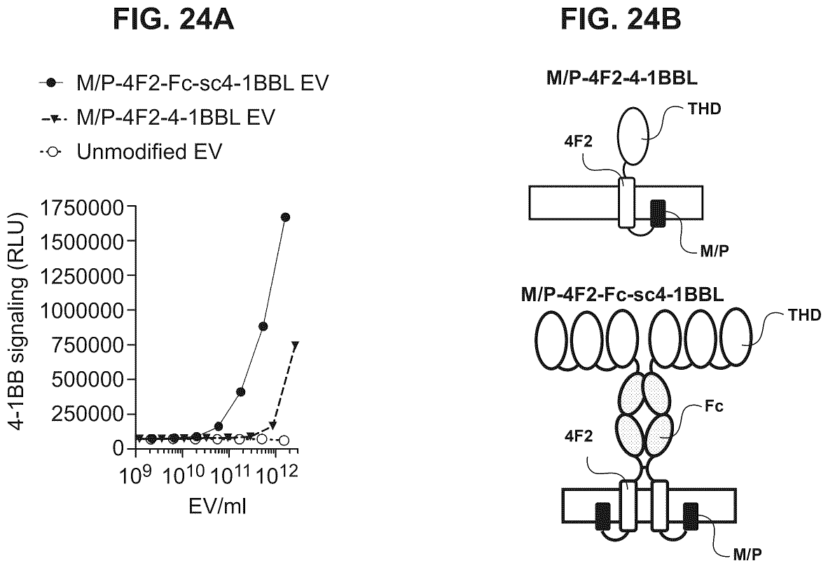

FIG. 24A shows 4-1BB agonism (i.e., signaling) as indicated in relative light units (RLU) of Promega 4-1BB Jurkat effector cells induced by SEC purified extracellular vesicles displaying embodiments of fusion polypeptide presented herein (e.g., M/P-4F2-Fc-sc4-1BBL or M/P-4F2-4-1BBL), or SEC purified unmodified extracellular vesicles purified from unmodified Flp-In 293 cells. SEC purified extracellular vesicles expressing the presented fusion polypeptide embodiments herein or SEC purified Flp-In derived extracellular vesicles were added to Promega Bioassay 4-1BB effector Jurkat cells. 4-1BB agonist signaling in Jurkat cells was substantially higher when purified extracellular vesicles displaying the M/P-4F2-Fc-sc4-1BBL fusion polypeptide was added to the bioassay when compared to unmodified EVs or EVs displaying fusion polypeptide embodiment M/P-4F2-4-1BBL, demonstrating empirically that M/P-4F2-Fc-sc4-1BBL is a preferred embodiment of a 4-1BBL fusion polypeptide. 4-1BB agonism was detected using genetically engineered Jurkat T cells that expresses human 4-1BB and a luciferase reporter driven by a response element that can respond to 4-1BB agonism. The Jurkat cells were then treated with a luciferase substrate and enzymatic activity was measured using a plate fluorometer. RLU, relative fluorescence units. 4-1BB agonist bioassays were performed using the Promega 4-1BB Bioassay kit essentially according to the manufacturer's instructions, the contents of which is incorporated herein by reference in its entirety (promega.com/products/reporter-bioassays/immune-checkpoint-bioassays/4-1bb-bioassay/?catNum=JA2351). In some experiments, the Promega 4-1BB Bioassay was modified to evaluate agonism by various embodiments of extracellular vesicles displaying fusion polypeptide, or unmodified extracellular vesicles purified from cells, or extracellular vesicles displaying native full length 4-1BBL. FIG. 24B shows schematic diagrams of the presented extracellular vesicle embodiments presented in FIG. 24A.

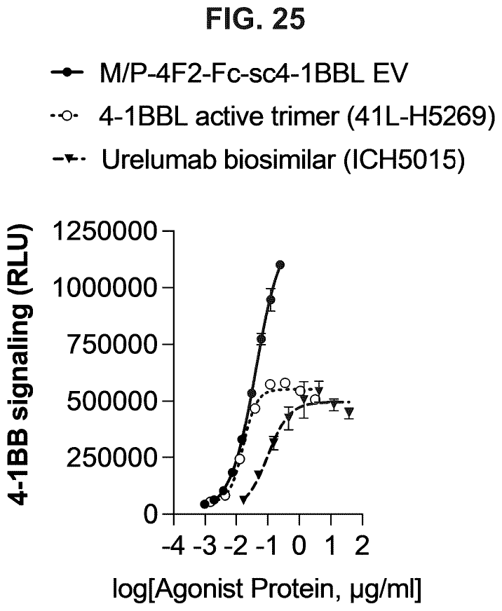

FIG. 25 shows 4-1BB agonism (i.e., signaling) as indicated in relative light units (RLU) of Jurkat 4-1BB effector cells induced by SEC purified extracellular vesicles displaying a preferred embodiment of fusion polypeptide M/P-4F2-Fc-sc4-1BBL, or a recombinant Fc tagged soluble ligand 4-1BBL trimer (Acro Biosystems, 41L-H5269), or Urelumab antibody biosimilar (Ichorbio, ICH5015) at the concentrations presented herein. The agonist protein concentration of M/P-4F2-Fc-sc4-1BBL is the concentration of 4-1BBL as determined by 4-1BBL ELISA immunoassay. SEC purified extracellular vesicles displaying the M/P-4F2-Fc-sc4-1BBL fusion polypeptide preferred embodiment presented herein, or a recombinant Fc tagged ligand trimer, or Urelumab antibody biosimilar, were added to Promega Bioassay 4-1BB effector Jurkat cells. Substantially higher 4-1BB agonist signaling in 4-1BB Jurkat cells was observed when purified extracellular vesicles displaying the exemplary and preferred embodiment M/P-4F2-Fc-sc4-1BBL fusion polypeptide was used in the bioassay when compared to 4-1BBL active trimer or Urelumab biosimilar. The results show empirically that EVs displaying M/P-4F2-Fc-sc4-1BBL fusion polypeptide are substantially more agonistic than Urelumab or a soluble 4-1BBL trimer.

Referring to FIG. 25, when compared on a 1:1 basis per 4-1BBL molecule, the purified extracellular vesicles displaying a M/P-4F2-Fc-sc4-1BBL fusion polypeptide have several orders magnitude higher agonist signaling when compared to Urelumab or a soluble 4-1BBL trimer polypeptide. For example, extracellular vesicles displaying the M/P-4F2-Fc-sc4-1BBL fusion polypeptide are at 10, 20, 30, 40, 50, 60, 70, 80, 90, 100, 1000, 10000, or more times more potent in inducing 4-1BB agonism than Urelumab. For example, extracellular vesicles displaying the M/P-4F2-Fc-sc4-1BBL fusion polypeptide are at 1, 2, 3, 4, 5, 6, 7, 8, 9, 10, 20, 30, 40, 50, 60, 70, 80, 90, 100, 1000, 10000, or more times more effective in inducing 4-1BB agonism than Urelumab. When compared on a 1:1 basis per 4-1BBL molecule, the purified extracellular vesicles displaying a M/P-4F2-Fc-sc4-1BBL fusion polypeptide are several orders magnitude agonistic compared to a recombinant Fc tagged soluble ligand 4-1BBL trimer (Acro Biosystems). For example, extracellular vesicles displaying a M/P-4F2-Fc-sc4-1BBL fusion polypeptide are at 10, 20, 30, 40, 50, 60, 70, 80, 90, 100, 1000, 10000, or more times more potent in inducing 4-1BB agonism than a recombinant Fc tagged soluble ligand 4-1BBL trimer. For example, extracellular vesicles displaying a M/P-4F2-Fc-sc4-1BBL fusion polypeptide are at 1, 2, 3, 4, 5, 6, 7, 8, 9, 10, 20, 30, 40, 50, 60, 70, 80, 90, 100, 1000, 10000, or more times more effective in inducing 4-1BB agonism than a recombinant Fc tagged soluble ligand 4-1BBL trimer.

4-1BB agonism was detected using genetically engineered Jurkat T cells that expresses human 4-1BB and a luciferase reporter driven by a response element that can respond to 4-1BB agonism. The Jurkat cells were then treated with a luciferase substrate and enzymatic activity was measured using a plate fluorometer. RLU, relative fluorescence units. 4-1BB agonist bioassays were performed using the Promega 4-1BB Bioassay kit essentially according to the manufacturer's instructions, the contents of which is incorporated herein by reference in its entirety (promega.com/products/reporter-bioassays/immune-checkpoint-bioassays/4-1bb-bioassay/?catNum=JA2351). In some experiments, the Promega 4-1BB Bioassay was modified to evaluate agonism by extracellular vesicles displaying fusion polypeptides, or unmodified extracellular vesicles purified from cells, or extracellular vesicles displaying native 4-1BBL.

### Example 9. GITR Agonism

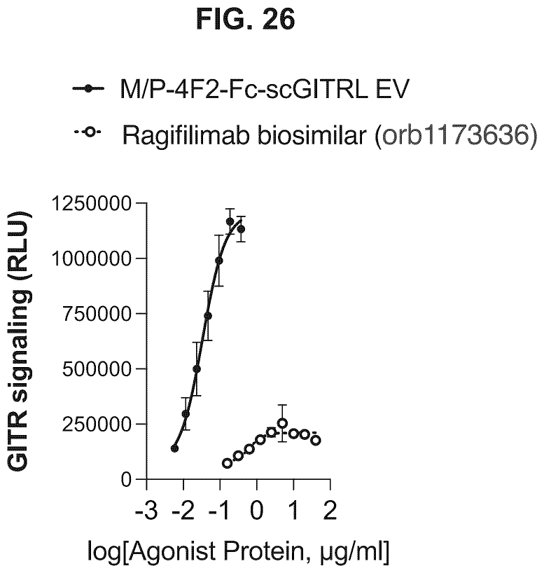

FIG. 26 shows GITR agonism (i.e., signaling) as indicated in relative light units (RLU) of Jurkat GITR effector cells induced by SEC purified extracellular vesicles displaying a preferred embodiment of fusion polypeptide M/P-4F2-Fc-scGITRL, or Ragifilimab antibody biosimilar (biorbyt, orb1173636) at the concentrations presented herein. The agonist protein concentration of M/P-4F2-Fc-scGITRL is the concentration of GITRL as determined by GITRL ELISA immunoassay. SEC purified extracellular vesicles displaying the M/P-4F2-Fc-scGITRL fusion polypeptide preferred embodiment presented herein, or Ragifilimab antibody biosimilar, were added to Promega Bioassay GITR effector Jurkat cells. Substantially higher GITR agonist signaling in GITR Jurkat cells was observed when purified extracellular vesicles displaying the exemplary and preferred embodiment M/P-4F2-Fc-scGITRL fusion polypeptide was used in the bioassay when compared to Ragifilimab biosimilar. The results show empirically that EVs displaying M/P-4F2-Fc-scGITRL fusion polypeptide are substantially more agonistic than Ragifilimab.

Referring to FIG. 26, when compared on a 1:1 basis per GITRL molecule, the purified extracellular vesicles displaying a M/P-4F2-Fc-scGITRL fusion polypeptide have several orders magnitude higher agonist signaling when compared to Ragifilimab. For example, extracellular vesicles displaying the M/P-4F2-Fc-scGITRL fusion polypeptide are at 10, 20, 30, 40, 50, 60, 70, 80, 90, 100, 1000, 10000, or more times more potent in inducing GITR agonism than Ragifilimab. For example, extracellular vesicles displaying a M/P-4F2-Fc-scGITRL fusion polypeptide are at 1, 2, 3, 4, 5, 6, 7, 8, 9, 10, 20, 30, 40, 50, 60, 70, 80, 90, 100, 1000, 10000, or more times more effective in inducing GITR agonism than Ragifilimab.

GITR agonism was detected using genetically engineered Jurkat T cells that expresses human GITR and a luciferase reporter driven by a response element that can respond to GITR agonism. The Jurkat cells were then treated with a luciferase substrate and enzymatic activity was measured using a plate fluorometer. RLU, relative fluorescence units. GITR agonist bioassays were performed using the Promega GITR Bioassay kit essentially according to the manufacturer's instructions, the contents of which is incorporated herein by reference in its entirety (promega.com/products/reporter-bioassays/immune-checkpoint-bioassays/gitr-bioassays/?catNum=JA2291).

### Example 10. OX40 Agonism

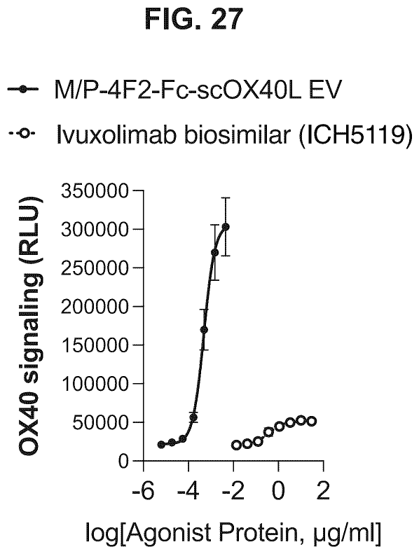

FIG. 27 shows OX40 agonism (i.e., signaling) as indicated in relative light units (RLU) of Jurkat OX40 effector cells induced by SEC purified extracellular vesicles displaying a preferred embodiment of fusion polypeptide M/P-4F2-Fc-scOX40L, or Ivuxolimab antibody biosimilar (Ichorbio, ICH5119) at the concentrations presented herein. The agonist protein concentration of M/P-4F2-Fc-scOX40L is the concentration of OX40L as determined by OX40L ELISA immunoassay. SEC purified extracellular vesicles displaying the M/P-4F2-Fc-scOX40L fusion polypeptide preferred embodiment presented herein, or Ivuxolimab antibody biosimilar, were added to Promega Bioassay OX40 effector Jurkat cells. Substantially higher OX40 agonist signaling in OX40 Jurkat cells was observed when purified extracellular vesicles displaying the exemplary and preferred embodiment M/P-4F2-Fc-scOX40L fusion polypeptide was used in the bioassay when compared to Ivuxolimab biosimilar. The results show empirically that EVs displaying M/P-4F2-Fc-scOX40L fusion polypeptide are substantially more agonistic than Ivuxolimab.

Referring to FIG. 27, when compared on a 1:1 basis per OX40L molecule, the purified extracellular vesicles displaying a M/P-4F2-Fc-scOX40L fusion polypeptide have several orders magnitude higher agonist signaling when compared to Ivuxolimab. For example, extracellular vesicles displaying the M/P-4F2-Fc-scOX40L fusion polypeptide are at 10, 20, 30, 40, 50, 60, 70, 80, 90, 100, 1000, 10000, or more times more potent in inducing OX40 agonism than Ivuxolimab. For example, extracellular vesicles displaying a M/P-4F2-Fc-scOX40L fusion polypeptide are at 1, 2, 3, 4, 5, 6, 7, 8, 9, 10, 20, 30, 40, 50, 60, 70, 80, 90, 100, 1000, 10000, or more times more effective in inducing OX40 agonism than Ivuxolimab.

OX40 agonism was detected using genetically engineered Jurkat T cells that expresses human OX40 and a luciferase reporter driven by a response element that can respond to OX40 agonism. The Jurkat cells were then treated with a luciferase substrate and enzymatic activity was measured using a plate fluorometer. RLU, relative fluorescence units. OX40 agonist bioassays were performed using the Promega OX40 Bioassay kit essentially according to the manufacturer's instructions, the contents of which is incorporated herein by reference in its entirety (promega.com/products/reporter-bioassays/immune-checkpoint-bioassays/ox40-bioassays/?catNum=JA2191).

### Example 11. Specificity of 4-1BB Agonism

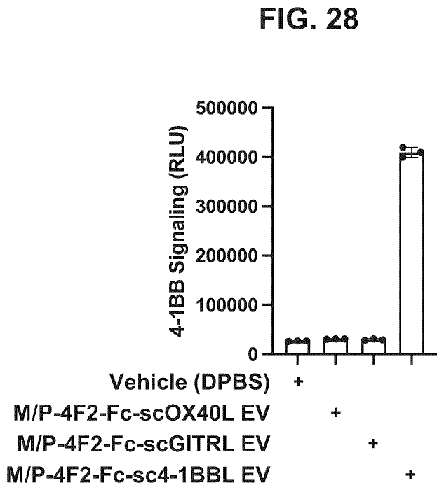

FIG. 28 shows 4-1BB agonism (i.e., signaling) as indicated in relative light units (RLU) of Jurkat 4-1BB effector cells induced by SEC purified extracellular vesicles displaying M/P-4F2-Fc-sc4-1BBL, or M/P-4F2-Fc-scGITRL, or M/P-4F2-Fc-scOX40L fusion polypeptide embodiments presented herein, or vehicle (DPBS). SEC purified extracellular vesicles displaying M/P-4F2-Fc-sc4-1BBL, or M/P-4F2-Fc-scGITRL, or M/P-4F2-Fc-scOX40L fusion polypeptide embodiments presented herein, were added to Promega Bioassay 4-1BB effector Jurkat cells. 4-1BB agonist signaling in Jurkat cells was observed when purified extracellular vesicles displaying the exemplary and preferred 4-1BB agonist embodiment M/P-4F2-Fc-sc4-1BBL fusion polypeptide was added to the bioassay. Agonism of 4-1BB target receptor was not observed when vehicle (DPBS) or extracellular vesicles displaying M/P-4F2-Fc-scGITR or M/P-4F2-Fc-scOX40L fusion polypeptide embodiments were added to the Promega Bioassay 4-1BB effector Jurkat cells, indicating that the 4-1BB agonist signaling induced by extracellular vesicles displaying M/P-4F2-Fc-sc4-1BBL fusion polypeptide is specific for the 4-1BB target receptor to 4-1BBL fusion polypeptide interaction.

4-1BB agonism was detected using genetically engineered Jurkat T cells that expresses human 4-1BB target receptor and a luciferase reporter driven by a response element that can respond to 4-1BB target receptor agonism. The Jurkat cells were then treated with a luciferase substrate and enzymatic activity was measured using a plate fluorometer. RLU, relative fluorescence units. 4-1BB agonist bioassays were performed using the Promega 4-1BB Bioassay kit essentially according to the manufacturer's instructions, the contents of which are incorporated herein by reference in their entirety (promega.com/products/reporter-bioassays/immune-checkpoint-bioassays/4-1bb-bioassay/?catNum=JA2351). In some experiments, the Promega 4-1BB Bioassay was modified to evaluate 4-1BB target receptor agonism by extracellular vesicles displaying one or more fusion polypeptide embodiment, or unmodified extracellular vesicles purified from cells, or extracellular vesicles displaying native full length 4-1BBL.

### Example 12. Specificity of GITR Agonism

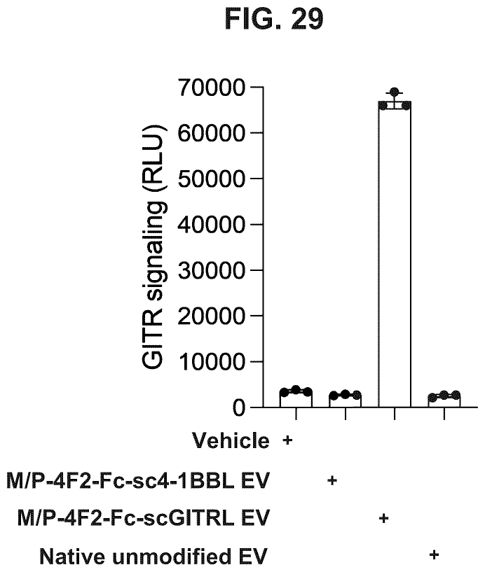

FIG. 29 shows GITR agonism (i.e., signaling) as indicated in relative light units (RLU) of Jurkat GITR effector cells induced by SEC purified extracellular vesicles displaying M/P-4F2-Fc-scGITRL, or M/P-4F2-Fc-sc4-1BBL fusion polypeptide embodiments presented herein, unmodified extracellular vesicles or vehicle (DPBS). SEC purified extracellular vesicles displaying M/P-4F2-Fc-sc4-1BBL or M/P-4F2-Fc-scGITRL fusion polypeptide embodiments presented herein, were added to Promega Bioassay GITR effector Jurkat cells. GITR agonist signaling in Jurkat cells was observed when purified extracellular vesicles displaying the exemplary and preferred GITR agonist embodiment M/P-4F2-Fc-scGITRL fusion polypeptide was added to the bioassay. Agonism of GITR target receptor was not observed when vehicle (DPBS), unmodified extracellular vesicles or extracellular vesicles displaying M/P-4F2-Fc-sc4-1BBL fusion polypeptide embodiment were added to the Promega Bioassay GITR effector Jurkat cells, indicating that the GITR agonist signaling induced by extracellular vesicles displaying M/P-4F2-Fc-scGITRL fusion polypeptide is specific for the GITR target receptor to GITRL fusion polypeptide interaction.

GITR agonism was detected using genetically engineered Jurkat T cells that expresses human GITR target receptor and a luciferase reporter driven by a response element that can respond to GITR target receptor agonism. The Jurkat cells were then treated with a luciferase substrate and enzymatic activity was measured using a plate fluorometer. RLU, relative fluorescence units. GITR agonist bioassays were performed using the Promega GITR Bioassay kit essentially according to the manufacturer's instructions, the contents of which are incorporated herein by reference in their entirety (promega.com/products/reporter-bioassays/immune-checkpoint-bioassays/gitr-bioassays/?catNum=JA2291).

### Example 13. Specificity of OX40 Agonism

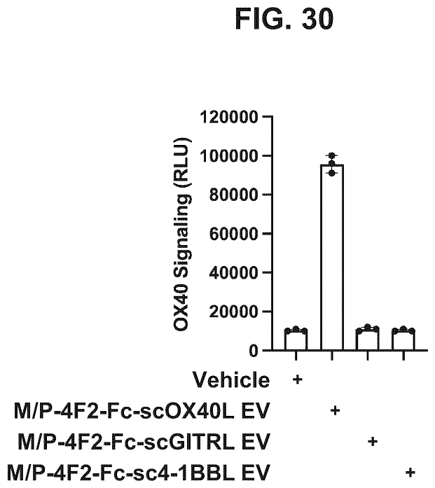

FIG. 30 shows OX40 agonism (i.e., signaling) as indicated in relative light units (RLU) of Jurkat OX40 effector cells induced by SEC purified extracellular vesicles displaying M/P-4F2-Fc-scOX40, M/P-4F2-Fc-scGITRL, or M/P-4F2-Fc-sc4-1BBL fusion polypeptide embodiments presented herein, or vehicle (DPBS). SEC purified extracellular vesicles displaying M/P-4F2-Fc-scOX40, M/P-4F2-Fc-scGITRL, or M/P-4F2-Fc-sc4-1BBL fusion polypeptide embodiments presented herein, were added to Promega Bioassay OX40 effector Jurkat cells. OX40 agonist signaling in Jurkat cells was observed when purified extracellular vesicles displaying the exemplary and preferred OX40 agonist embodiment M/P-4F2-Fc-scOX40 fusion polypeptide was added to the bioassay. Agonism of OX40 target receptor was not observed when vehicle (DPBS) or extracellular vesicles displaying M/P-4F2-Fc-scGITRL or M/P-4F2-Fc-sc4-1BBL fusion polypeptide embodiments were added to the Promega OX40 Bioassay effector Jurkat cells, indicating that the OX40 agonist signaling induced by extracellular vesicles displaying M/P-4F2-Fc-scOX40 fusion polypeptide is specific for the OX40 target receptor to OX40L fusion polypeptide interaction.

OX40 agonism was detected using genetically engineered Jurkat T cells that expresses human OX40 target receptor and a luciferase reporter driven by a response element that can respond to OX40 target receptor agonism. The Jurkat cells were then treated with a luciferase substrate and enzymatic activity was measured using a plate fluorometer. RLU, relative fluorescence units. OX40 agonist bioassays were performed using the Promega OX40 Bioassay kit essentially according to the manufacturer's instructions, the contents of which are incorporated herein by reference in their entirety (promega.com/products/reporter-bioassays/immune-checkpoint-bioassays/ox40-bioassays/?catNum=JA2191).

### Example 14. Stimulation of Antigen-Specific Memory T Cell Expansion

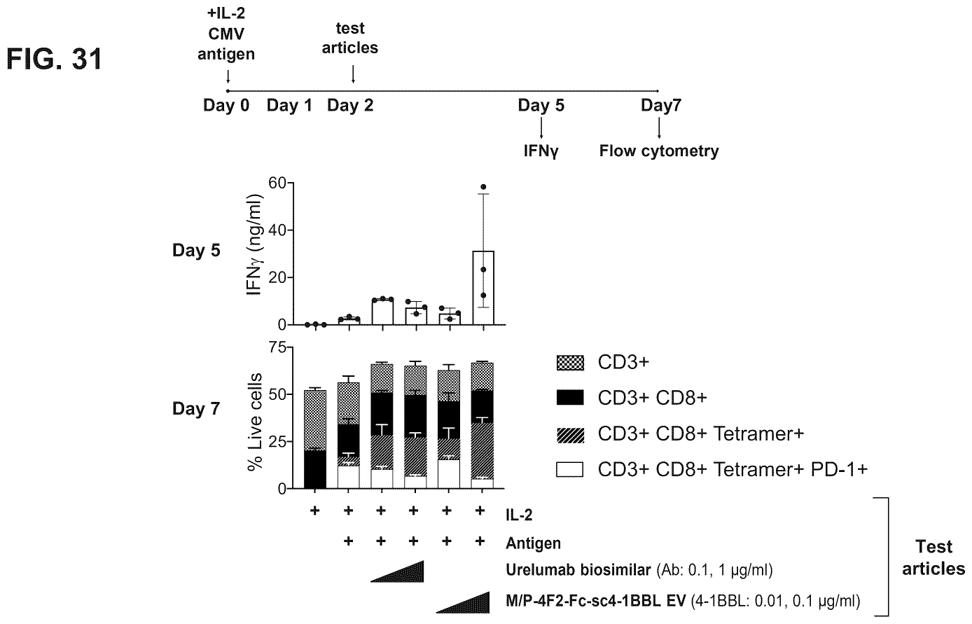

FIG. 31 shows modulation of antigen-specific memory T cell expansion and function by extracellular vesicles displaying M/P-4F2-Fc-sc4-1BBL fusion polypeptide in a Cytomegalovirus (CMV) recall assay. The assay was performed essentially as described in Kleiman E, Sierra G, Mao B, Magcase D, George M V, Daftarian P M. Adenosine-related small molecules show utility of recall antigen assay to screen compounds for off-target effects on memory T cells. Sci Rep. 2021 May 5; 11 (1): 9561. doi: 10.1038/s41598-021-88965-3. PMID: 33953256; PMCID: PMC8100288, the contents of which are incorporated herein by reference in their entirety.

Briefly healthy HLA-A*02:01 Cytomegalovirus (CMV) seropositive donor human Peripheral Blood Mononuclear Cells (PBMCs) were seeded at 500,000 cells per well and stimulated on day 0 with CMV pp65 recall antigen peptide NLVPMVATV (2 μg/mL final concentration) to expand antigen-specific memory T cells for 7 days or 500,000 cells per well of unstimulated cells. On day 2, IL-2 (1 mM final concentration) was added to the wells and the indicated test articles were added to assess their effects on T cell expansion. Two concentrations of SEC purified extracellular vesicles displaying M/P-4F2-Fc-sc4-1BBL fusion polypeptide were used: 10 μg/mL and 100 μg/mL. Two concentrations of Urelumab antibody biosimilar (Ichorbio, ICH5015) were used: 0.1 μg/mL and 1 μg/mL. 100 μl supernatant was collected on day 5, after which the medium was replenished for all wells. Day 5 supernatant was analyzed by Meso Scale Discovery (MSD) immunoassay for Interferon gamma cytokine. On day 7 cells were treated with 50 nM Dasatinib and harvested for Flow cytometry analysis. Flow cytometry analysis was performed on day 7 for iTAg Tetramer-HLA-A*02:01 CMV pp65 (NLVPMVATV), CD3, CD8, PD-1, and live/dead cell staining.

Referring to FIG. 31 Day 7 results, in the CMV recall assay presented herein, as a proportion of total live cells, treatment of CMV pp65 activated HLA-A*02:01 CMV seropositive PBMCs with Urelumab biosimilar antibody resulted a substantial increase of antigen specific CD8+ T-cells and a substantial decrease of antigen specific CD8+PD1+ T-cells. As a proportion of total live cells, treatment of CMV pp65 activated HLA-A*02:01 CMV seropositive PBMCs with extracellular vesicles displaying M/P-4F2-Fc-sc4-1BBL fusion polypeptide caused a substantial increase of antigen specific cells CD8+ T-cells, and a substantial decrease of CD8+PD1+ T-cells, wherein the increase of antigen specific CD8+ T-cells by extracellular vesicles displaying M/P-4F2-Fc-sc4-1BBL fusion polypeptide was substantially greater than the increase of antigen specific CD8+ T-cells by Urelumab, and wherein the decrease of CD8+PD1+ T-cells by extracellular vesicles displaying M/P-4F2-Fc-sc4-1BBL fusion polypeptide was substantially a greater decrease than the decrease of CD8+PD1+ T-cells cells by Urelumab.

In the CMV recall assay presented herein, as a proportion of total live cells, extracellular vesicles displaying M/P-4F2-Fc-sc4-1BBL fusion polypeptide substantially enhanced the expansion of antigen specific memory CD8+ T-cells while substantially decreased the relative proportion of CD8+PD-1+ T-cells in a dose dependent manner, wherein a higher dose of extracellular vesicles displaying M/P-4F2-Fc-sc4-1BBL fusion polypeptide caused a substantially greater increase of CD8+ T-cells expansion and a substantially greater decrease of CD8+PD-1+ T-cells. CD8+PD-1+ T-cells are considered putatively exhausted T-cells herein. The results show that extracellular vesicles displaying the M/P-4F2-Fc-sc4-1BBL fusion polypeptide substantially enhanced proliferation of antigen specific T-cells. The results show that extracellular vesicles displaying the M/P-4F2-Fc-sc4-1BBL fusion polypeptide substantially stimulate proliferation of antigen specific T-cells. The results show that extracellular vesicles displaying the M/P-4F2-Fc-sc4-1BBL fusion polypeptide have a substantial co-stimulatory agonist effect on antigen specific T-cells. The results show that extracellular vesicles displaying the M/P-4F2-Fc-sc4-1BBL fusion polypeptide have a substantial co-stimulatory agonist effect on immune cells.

The results show that extracellular vesicles displaying the M/P-4F2-Fc-sc4-1BBL fusion polypeptide substantially decrease exhaustion of antigen specific T-cells. The results show that extracellular vesicles displaying the M/P-4F2-Fc-sc4-1BBL fusion polypeptide substantially decrease the proportion of exhausted antigen specific T-cells.

The results show that on a per molecule basis of a 4-1BB agonist molecule (for example, extracellular vesicle displaying 4-1BB agonist M/P-4F2-Fc-sc4-1BBL fusion polypeptide versus 4-1BB agonist Urelumab) extracellular vesicles displaying the M/P-4F2-Fc-sc4-1BBL fusion polypeptide is substantially superior to Urelumab in enhancing or stimulating proliferation of antigen specific T-cells. The results show that on a per molecule basis of a 4-1BB agonist molecule (for example, extracellular vesicle displaying 4-1BB agonist M/P-4F2-Fc-sc4-1BBL fusion polypeptide versus 4-1BB agonist Urelumab) extracellular vesicles displaying the M/P-4F2-Fc-sc4-1BBL fusion polypeptide are substantially superior to Urelumab in decreasing the proportion of putatively exhausted T-cells (for example, CD8+PD-1+ T-cells).

Referring to FIG. 31 Day 7 results, CMV recall assay results showed that extracellular vesicles displaying the M/P-4F2-Fc-sc4-1BBL fusion polypeptide substantially increased interferon gamma expression in a dose dependent manner, wherein higher concentration of extracellular vesicles displaying the M/P-4F2-Fc-sc4-1BBL fusion polypeptide showed substantially higher interferon gamma expression than did lower concentrations of extracellular vesicles displaying the M/P-4F2-Fc-sc4-1BBL fusion polypeptide. It is demonstrated herein that extracellular vesicles displaying the M/P-4F2-Fc-sc4-1BBL can substantially stimulate interferon gamma expression by cells, for example human PBMCs.

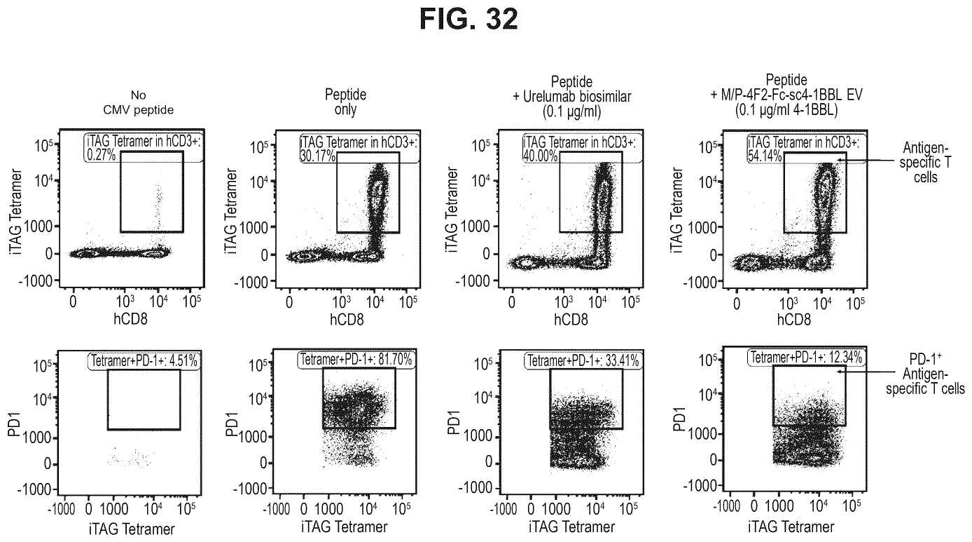

FIG. 32 shows example flow cytometry plots for antigen-specific T cells (CD3⁺/CD8⁺/MHC-peptide tetramer+) and PD-1⁺ cells within the antigen specific T cells.

### Example 15. Stimulation of Cancer Cell Killing by Purified T Cells

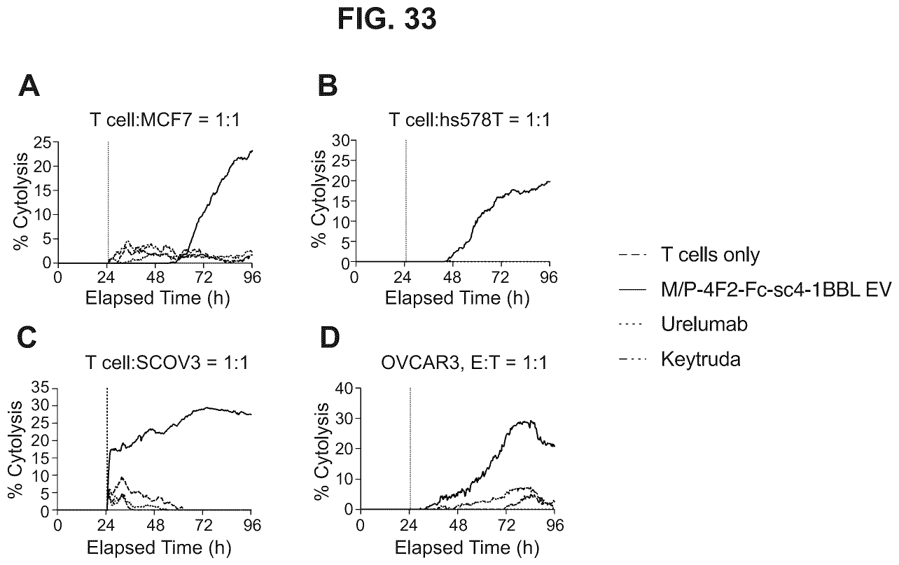

FIG. 33 shows that extracellular vesicles displaying the M/P-4F2-Fc-sc4-1BBL fusion polypeptide induce T cell-mediated cytotoxic activity. T cells purified from the PBMCs of a healthy HLA-A*02:01 CMV seropositive donor, were co-cultured with one of four target cell lines: (A) MCF7, (B) Hs578T, (C) SKOV3, (D) OVCAR3. One day before co-culture (Day-1), target cells were seeded into 96-well E-plates at 5,000 cells per well. On Day 0, isolated effector T cells were co-cultured with target cells at an effector to target (E:T) ratio of 1:1. Extracellular vesicles displaying the M/P-4F2-Fc-sc4-1BBL fusion polypeptide (containing ˜32 ng/ml 4-1BBL) were added to specified wells to assess their effect on T-cell mediated anti-tumor activity. Urelumab (anti-CD137) or Keytruda (Pembrolizumab, anti-PD-1) were added at lug/mL as controls. Continuous impedance monitoring using the Agilent xCELLigence RTCA system allowed for confluency measurements every 15 minutes to determine % cytolysis of target cells.

### Example 16. Treatment of Cancer

Extracellular vesicles engineered to display a fusion polypeptide embodiment presented herein were evaluated as a cancer therapy. An engineered extracellular vesicle embodiment displaying fusion polypeptide M/P-4F2-Fc-sc4-1BBL was evaluated as a cancer therapy.

FIG. 34 shows intratumoral treatment of subcutaneous MC38 Colon Carcinoma Model in B-h4-1BB mice. B-h4-1BB mice were subcutaneously injected with MC38 tumor cells (5×10⁵) with 0.1 mL PBS in the right front flank for tumor development. Tumor-bearing animals were randomly enrolled into four study groups of eight animal when the mean tumor size reached 100 mm³. Starting on the same day of grouping (day 0) 50 μl of a test article was administered intratumorally every three days for a total of 9 doses. Animals with tumor volumes exceeding 2000 mm³ were humanely euthanized. Tumor volumes, animal weight and survival were monitored up to 35 days. Test articles were: (A) vehicle (PBS), (B) Flp-In HEK 293 derived extracellular vesicles, (C) Urelumab antibody biosimilar (6 μg/dose), and (D) SEC purified extracellular vesicles displaying 4-1BB agonist fusion polypeptide M/P-4F2-Fc-sc4-1BBL (70 ng 4-1BB/dose). (E) Shows mean+/−SEM tumor volumes. Tumor growth during treatment with extracellular vesicles displaying the fusion polypeptide M/P-4F2-Fc-sc4-1BBL (p=0.0025), but not Urelumab, was significantly different from Vehicle (i.e., phosphate buffered saline, PBS) as determined by the Friedman test through D25 (N=8).

Referring to FIG. 34 At day 35 post grouping, one mouse remained alive in the group treated with DPBS, two mice remained alive in the group treated with unmodified extracellular vesicles, six mice remained alive in the group treated with extracellular vesicles displaying M/P-4F2-Fc-sc4-1BBL fusion polypeptide embodiment, and three mice survived in the group treated with Urelumab antibody biosimilar. Between days 7 and 14, and between days 18 and 21 post grouping, tumor volumes decreased for mice treated with extracellular vesicles displaying M/P-4F2-Fc-sc4-1BBL fusion polypeptide embodiment. The MC38 results presented herein demonstrate that extracellular vesicles displaying the M/P-4F2-Fc-sc4-1BBL fusion polypeptide embodiment substantially decrease tumor volumes growth rate and are an effective therapeutic for the treatment of cancer. The MC38 results presented herein demonstrate that extracellular vesicles displaying 4-1BB agonist M/P-4F2-Fc-sc4-1BBL fusion polypeptide embodiment are substantially superior to a Urelumab 4-1BB agonist in stimulating the immune system and decreasing cancer tumor volume growth rate. See FIG. 34E.

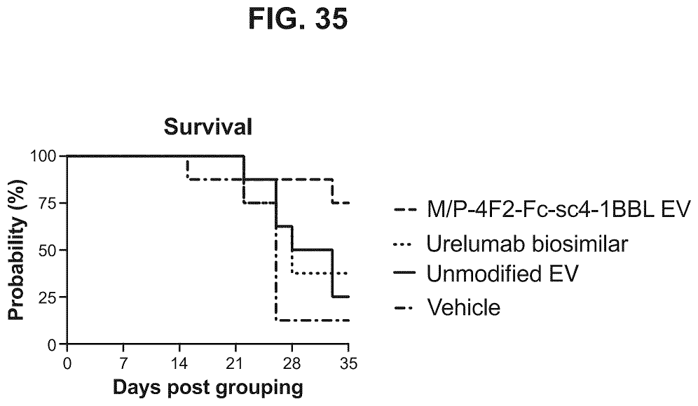

FIG. 35 shows Kaplan-Meyer survival curve for intratumoral treatment of subcutaneous MC38 Colon Carcinoma Model in B-h4-1BB mice. Significantly improved survival in mice treated with extracellular vesicles displaying the M/P-4F2-Fc-sc4-1BBL fusion polypeptide (p=0.0147), but not Urelumab treated mice (p=0.2973), compared to vehicle treated mice by Log-rank (Mantel-Cox) test. The MC38 results presented herein demonstrate that extracellular vesicles displaying 4-1BB agonist M/P-4F2-Fc-sc4-1BBL fusion polypeptide embodiment are substantially superior to a Urelumab 4-1BB agonist in stimulating the immune system and substantially lengthening duration of survival.

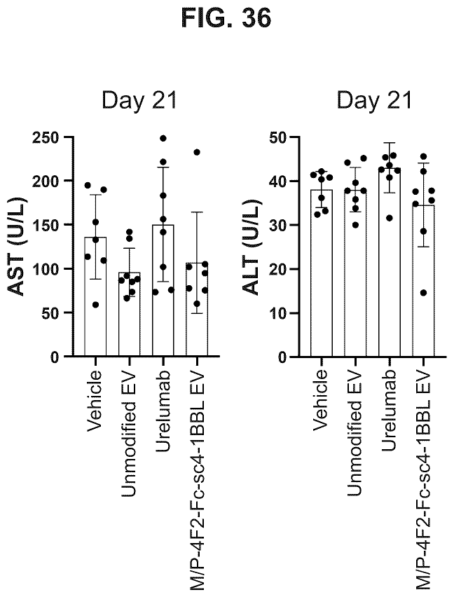

FIG. 36 shows intratumoral treatment of subcutaneous MC38 Colon Carcinoma Model in B-h4-1BB mice. At day 21 liver enzymes were measured. no statistically significant elevation of liver enzymes in animals treated with extracellular vesicles displaying the M/P-4F2-Fc-sc4-1BBL fusion polypeptide compared to Vehicle, unmodified extracellular vesicles or Urelumab.

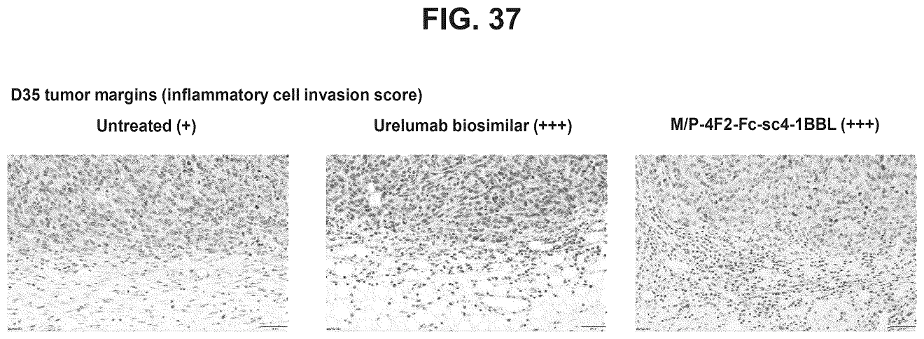

FIG. 37 shows hematoxylin and eosin staining of D35 tumor margin sections. Tumors treated with Urelumab or extracellular vesicles displaying the M/P-4F2-Fc-sc4-1BBL showed greater infiltration of inflammatory cells.

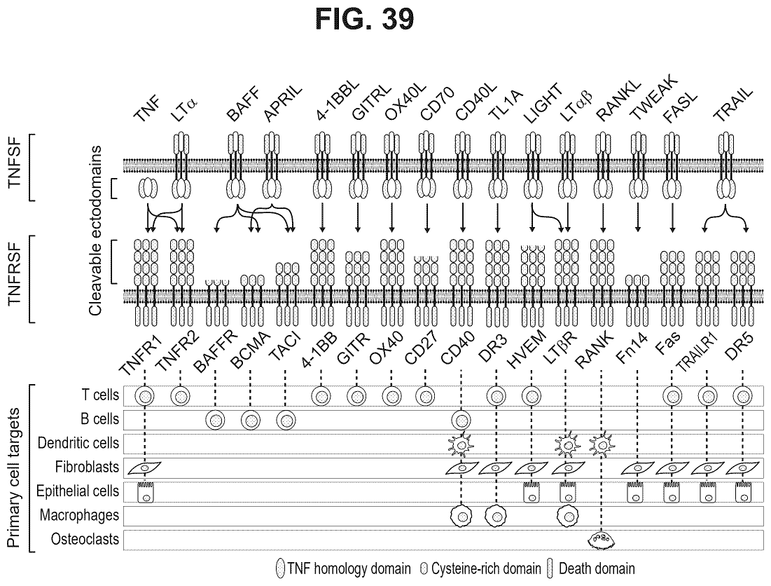

FIG. 39 illustrates TNF superfamily ligands (TNFSF), TNF receptor superfamily (TNFRSF), and their primary cellular targets. TNFSF are active primarily as non-covalently associated homotrimers and can be soluble or membrane-expressed. TNFRSF contain variable numbers of cysteine-rich domains in their ligand-binding extracellular regions. TNFRSF are mainly membrane-expressed and serve as critical regulators of human immunity. Activating, or agonizing, TNF receptors to enhance immunity has proven to be far more elusive than inhibiting TNF receptors.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

1. An engineered extracellular vesicle comprising at least one fusion polypeptide, the fusion polypeptide comprising:
an agonistic multi-effector domain in an exterior position relative to a phospholipid bilayer of the extracellular vesicle;
a polypeptide linker; and
at least one vesicle targeting domain spanning at least partly through the phospholipid bilayer of the extracellular vesicle,
wherein the polypeptide linker is positioned between the multi-effector domain and the at least one vesicle targeting domain, and
wherein the agonistic multi-effector domain comprises at least one fragment from one or more proteins of interest.

2. The engineered extracellular vesicle of claim 1, wherein the at least one vesicle targeting domain is a Type II transmembrane protein or fragment thereof, or a multi-pass transmembrane protein or fragment thereof.

3. The engineered extracellular vesicle of claim 2, wherein the Type II transmembrane protein or fragment thereof comprises 4F2 (CD98 heavy chain) or CD298.

4. The engineered extracellular vesicle of claim 2, wherein the multi-pass transmembrane protein is a tetraspanin or fragment thereof.

5. The engineered extracellular vesicle of claim 2, wherein the at least one vesicle targeting domain further comprises at least one lipid anchoring domain.

6. The engineered extracellular vesicle of claim 5, wherein the at least one lipid anchoring domain further comprises a myristoylation and/or palmitoylation sequence.

7. The engineered extracellular vesicle of claim 2, wherein the polypeptide linker is positioned C-terminus relative to the at least one vesicle targeting domain.

8. The engineered extracellular vesicle of claim 2, wherein the polypeptide linker is positioned N-terminus relative to the agonistic multi-effector domain.

9-19. (canceled)

20. The engineered extracellular vesicle of claim 1, further comprising a linker between the agonistic multi-effector domain and the polypeptide linker.

21. The engineered extracellular vesicle of claim 1, further comprising a linker between the polypeptide linker and the at least one vesicle targeting domain.

22. The engineered extracellular vesicle of claim 1, further comprising a monomer linker between each monomer of the agonistic multi-effector domain.

23. The engineered extracellular vesicle of claim 1, wherein the agonistic multi-effector domain comprises at least three fragments from one or more protein of interest.

24. The engineered extracellular vesicle of claim 1, wherein each fragment of the agonistic multi-effector domain is a tumor necrosis factor (TNF) homology domain (THDs) or a fragment thereof.

25. The engineered extracellular vesicle of claim 24, wherein the THD is derived from a TNF superfamily member (TNFSF) selected from the group consisting of TNFα, TNFβ, TNFγ, ED1-A1, EDI-A2, GITRL, 4-1BBL, OX40L, LIGHT, CD27L, CD30L, CD40L, TRAIL, FASL, BAFF, APRIL, RANKL, TL1A, TWEAK or a fragment thereof.

26. The engineered extracellular vesicle of claim 20, wherein the linker between the agonistic multi-effector domain and the polypeptide linker, the linker between the polypeptide linker and the at least one vesicle targeting domain, or the linker between each fragment is each independently selected from the group consisting of ID, GSSG (SEQ ID NO: 154), G, GS, GGS, GGGS (SEQ ID NO: 218), GGGGS (SEQ ID NO:156), (GGGGS)n wherein n is an integer between 1 and 10, and combinations thereof.

27. The engineered extracellular vesicle claim 1, wherein the polypeptide linker comprises Fc or Fc mutein.

28. The engineered extracellular vesicle of claim 1, wherein the extracellular vesicle is an exosome.

29. A composition comprising a plurality of the engineered extracellular vesicle of claim 1, further comprising a pharmaceutically acceptable carrier.

30. (canceled)

31. An engineered extracellular vesicle comprising at least one fusion polypeptide, the fusion polypeptide comprising:
a signaling domain in an exterior position relative to a phospholipid bilayer of the extracellular vesicle;
a polypeptide linker; and
at least one vesicle targeting domain spanning at least partly through the phospholipid bilayer of the extracellular vesicle,
wherein the polypeptide linker is positioned between the signaling domain and the at least one vesicle targeting domain, and
wherein the signaling domain comprises a fragment from a protein of interest.

32. A method of manufacturing a population of the engineered extracellular vesicle of claim 1, comprising the steps of genetically engineering a cell line selected from the group consisting of HEK293, PER.C6, fibrosarcoma HT-1080, HuH7, and mesenchymal stem cells with a vector or gene encoding the agonistic multi-effector domain, the polypeptide linker, and the at least one vesicle targeting domain to produce a genetically engineered cell line; and generating, separating, and purifying the population of engineered extracellular vesicles from the genetically engineered cell line.


SEQUENCE LISTING (219 sequences)

SEQ ID NO: 1 - RNA, 2590 nt, Homo sapiens
  source 1..2590: mol_type = mRNA
ctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggtatcatgtagtatgtgaaatttattcttaaacgtgactactttatttctaaataagaaattccctacctgcttcctacaagcagttcagaatgccatgccttggttgtcctagtgtgaataattttcagctactttaaaattatattgtactttctcaagcatgtcatatcctttcctattagagtatctatattacttgttactgatttacctgaaggcaatctgattaatttctaggtttttaccatattcttgtcatcttgccaattacattttaagtgttagactagactaagatgtactagttgtatagaatataactagatttattatggcaatgtttattttgtcattttgcttcatctgttttgttgttgaagtactttaaatttcatacgttcatggcatttcactgtaaagactttaatgtgtatttcttaaaataaaactttttttcctccttaa

SEQ ID NO: 2 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 3 - RNA, 7796 nt, Homo sapiens
  source 1..7796: mol_type = mRNA
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 4 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 5 - RNA, 1936 nt, Homo sapiens
  source 1..1936: mol_type = mRNA
agaaccccgcggggtctgagcagcccagcgtgcccattccagcgcccgcgtccccgcagcatgccgcgcccccgcctgctggccgcgctgtgcggcgcgctgctctgcgcccccagcctcctcgtcgccctggatatctgttccaaaaacccctgccacaacggtggtttatgcgaggagatttcccaagaagtgcgaggagatgtcttcccctcgtacacctgcacgtgccttaagggctacgcgggcaaccactgtgagacgaaatgtgtcgagccactgggcctggagaatgggaacattgccaactcacagatcgccgcctcgtctgtgcgtgtgaccttcttgggtttgcagcattgggtcccggagctggcccgcctgaaccgcgcaggcatggtcaatgcctggacacccagcagcaatgacgataacccctggatccaggtgaacctgctgcggaggatgtgggtaacaggtgtggtgacgcagggtgccagccgcttggccagtcatgagtacctgaaggccttcaaggtggcctacagccttaatggacacgaattcgatttcatccatgatgttaataaaaaacacaaggagtttgtgggtaactggaacaaaaacgcggtgcatgtcaacctgtttgagacccctgtggaggctcagtacgtgagattgtaccccacgagctgccacacggcctgcactctgcgctttgagctactgggctgtgagctgaacggatgcgccaatcccctgggcctgaagaataacagcatccctgacaagcagatcacggcctccagcagctacaagacctggggcttgcatctcttcagctggaacccctcctatgcacggctggacaagcagggcaacttcaacgcctgggttgcggggagctacggtaacgatcagtggctgcaggtggacctgggctcctcgaaggaggtgacaggcatcatcacccagggggcccgtaactttggctctgtccagtttgtggcatcctacaaggttgcctacagtaatgacagtgcgaactggactgagtaccaggaccccaggactggcagcagtaagatcttccctggcaactgggacaaccactcccacaagaagaacttgtttgagacgcccatcctggctcgctatgtgcgcatcctgcctgtagcctggcacaaccgcatcgccctgcgcctggagctgctgggctgttagtggccacctgccacccccaggtcttcctgctttccatgggcccgctgcctcttggcttctcagcccctttaaatcaccatagggctggggactggggaaggggagggtgttcagaggcagcaccaccacacagtcacccctccctccctctttcccaccctccacctctcacgggccctgccccagcccctaagccccgtcccctaacccccagtcctcactgtcctgttttcttaggcactgagggatctgagtaggtctgggatggacaggaaagggcaaagtagggcgtgtggtttccctgcccctgtccggaccgccgatcccaggtgcgtgtgtctctgtctctcctagcccctctctcacacatcacattcccatggtggcctcaagaaaggcccggaagcgccaggctggagataacagcctcttgcccgtcggccctgcgtcggccctggggtaccatgtggccacaactgctgtggccccctgtccccaagacacttccccttgtctccctggttgcctctcttgccccttgtcctgaagcccagcgacacagaagggggtggggcgggtctatggggagaaagggagcgaggtcagaggagggcatgggttggcagggtgggcgtttggggccctctatgctggcttttcaccccagaggacacaggcagcttccaaaatatatttatcttcttcacgggaa

SEQ ID NO: 6 - protein, 387 aa, Homo sapiens
MPRPRLLAALCGALLCAPSLLVALDICSKNPCHNGGLCEEISQEVRGDVFPSYTCTCLKGYAGNHCETKCVEPLGLENGNIANSQIAASSVRVTFLGLQHWVPELARLNRAGMVNAWTPSSNDDNPWIQVNLLRRMWVTGVVTQGASRLASHEYLKAFKVAYSLNGHEFDFIHDVNKKHKEFVGNWNKNAVHVNLFETPVEAQYVRLYPTSCHTACTLRFELLGCELNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 7 - RNA, 1225 nt, Homo sapiens
  source 1..1225: mol_type = mRNA
agccgcctgcatctgtatccagcgccaggtcccgccagtcccagctgcgcgcgccccccagtcccgcacccgttcggcccaggctaagttagccctcaccatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagagtcagcttacatccctgagcaggaaagtttacccatgaagattggtgggattttttgtttgtttgttttgttttgtttgttgtttgttgtttgtttttttgccactaattttagtattcattctgcattgctagataaaagctgaagttactttatgtttgtcttttaatgcttcattcaatattgacatttgtagttgagcggggggtttggtttgctttggtttatattttttcagttgtttgtttttgcttgttatattaagcagaaatcctgcaatgaaaggtactatatttgctagactctagacaagatattgtacataaaagaatttttttgtctttaaatagatacaaatgtctatcaactttaatcaagttgtaacttatattgaagacaatttgatacataataaaaaattatgacaatgtcctgga

SEQ ID NO: 8 - protein, 228 aa, Homo sapiens
MPVKGGTKCIKYLLFGFNFIFWLAGIAVLAIGLWLRFDSQTKSIFEQETNNNNSSFYTGVYILIGAGALMMLVGFLGCCGAVQESQCMLGLFFGFLLVIFAIEIAAAIWGYSHKDEVIKEVQEFYKDTYNKLKTKDEPQRETLKAIHYALNCCGLAGGVEQFISDICPKKDVLETFTVKSCPDAIKEVFDNKFHIIGAVGIGIAVVMIFGMIFSMILCCAIRRNREMV

SEQ ID NO: 9 - RNA, 96 nt, Homo sapiens
  source 1..96: mol_type = mRNA
ttctacacaggagtctatattctgatcggagccggcgccctcatgatgctggtgggcttcctgggctgctgcggggctgtgcaggagtcccagtgc

SEQ ID NO: 10 - protein, 32 aa, Homo sapiens
FYTGVYILIGAGALMMLVGFLGCCGAVQESQC

SEQ ID NO: 11 - RNA, 2221 nt, Homo sapiens
  source 1..2221: mol_type = mRNA
gcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaa

SEQ ID NO: 12 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 13 - RNA, 1847 nt, Homo sapiens
  source 1..1847: mol_type = mRNA
agtcggctcgagtactccccgtaacgaggaggtgttctcggccgtcccacccttcactgccgtctccgggctgcgccgccggagccgggacgcgcctccgcagccctcgccgcctccatccccgcggccgcagctcctctcgccgtccgcgcgcacaccatgacgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgatgtgggttatgcttcagactctcaacgatgaggttccaaaataccgtgaccagattcctagcccaggactcatggtttttccaaaaccagtgaccgcattggaatatacattcagtaggtctgatccaacttcgtatgcagggtacattgaagaccttaagaagtttctaaaaccatatactttagaagaacagaagaacctcacagtctgtcctgatggagcactttttgaacagaagggtccagtttatgttgcatgtcagtttcctatttcattacttcaagcatgcagtggtatgaatgatcctgattttggctattctcaaggaaacccttgtattcttgtgaaaatgaacagaataattggattaaagcctgaaggagtgccaaggatagattgtgtttcaaagaatgaagatataccaaatgtagcagtttatcctcataatggaatgatagacttaaaatatttcccatattatgggaaaaaactgcatgttgggtatctacagccattggttgctgttcaggtcagctttgctcctaacaacactgggaaagaagtaacagttgagtgcaagattgatggatcagccaacctaaaaagtcaggatgatcgtgacaagtttttgggacgagttatgttcaaaatcacagcacgtgcatagtatgagtaggatatctccacagagtaaatgttgtgttgtctgtcttcattttgtaacagctggaccttccattctagaattatgagaccaccttggagaaaggtgtgtggtacatgacattgggttacatcataacgtgcttccagatcatagtgttcagtgtcctctgaagtaactgcctgttgcctctgctgccctttgaaccagtgtacagtcgccagatagggaccggtgaacacctgattccaaacatgtaggatgggggtcttgtcctctttttatgtggtttaattgccaagtgtctaaagcttaatatgccgtgctatgtaaatattttatggatataacaactgtcatattttgatgtcaacagagttttagggataaaatggtacccggccaacatcaagtgactttatagctgcaagaaatgtggtatgtggagaagttctgtatgtgaggaaggaaaaaaagaaaataaaagtgtgtttgaaaaatattatcttgggttctttgtaaaatttattttttacatgctgaattagcctcgatctttttgattaagagcacaaacttttttttgtaaaacatgtaaaaaaaaaaactgggattaatttttagtgttggaactgcctcttattttaggctgtagataaaatagcatttttaggttagccagtgtgactatgcacctaattttttatgagattaaattcataagacttaatttgtacaatagtttgtgaaatatcttgttactgcttttatttagcagactgtggactgtaataaagtatataaattgtgaaatataaaaacttggaacttattcaaagcttcaaagcaaa

SEQ ID NO: 14 - protein, 279 aa, Homo sapiens
MTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARA

SEQ ID NO: 15 - RNA, 4296 nt, Homo sapiens
  source 1..4296: mol_type = mRNA
gcacttgggcgttggaccccgcatcttattagcaaccagggagatttctccattttcctcttgtctacagtgcggctacaaatctgggatttttttattacttctttttttttcgaactacacttgggctcctttttttgtgctcgacttttccaccctttttccctccctcctgtgctgctgctttttgatctcttcgactaaaatttttttatccggagtgtatttaatcggttctgttctgtcctctccaccacccccacccccctccctccggtgtgtgtgccgctgccgctgttgccgccgccgctgctgctgctgctcgccccgtcgttacaccaacccgaggctctttgtttcccctcttggatctgttgagtttctttgttgaagaagccagcatgggtgcccagttctccaagaccgcagcgaagggagaagccgccgcggagaggcctggggaggcggctgtggcctcgtcgccttccaaagcgaacggacaggagaatggccacgtgaaggtaaacggcgacgcttcgcccgcggccgccgagtcgggcgccaaggaggagctgcaggccaacggcagcgccccggccgccgacaaggaggagcccgcggccgccgggagcggggcggcgtcgccctccgcggccgagaaaggtgagccggccgccgccgctgcccccgaggccggggccagcccggtagagaaggaggcccccgcggaaggcgaggctgccgagcccggctcgcccacggccgcggagggagaggccgcgtcggccgcctcctcgacttcttcgcccaaggccgaggacggggccacgccctcgcccagcaacgagaccccgaaaaaaaaaaagaagcgcttttccttcaagaagtctttcaagctgagcggcttctccttcaagaagaacaagaaggaggctggagaaggcggtgaggctgaggcgcccgctgccgaaggcggcaaggacgaggccgccgggggcgcagctgcggccgccgccgaggcgggcgcggcctccggggagcaggcagcggcgccgggcgaggaggcggcagcgggcgaggagggggcggcgggtggcgacccgcaggaggccaagccccaggaggccgctgtcgcgccagagaagccgcccgccagcgacgagaccaaggccgccgaggagcccagcaaggtggaggagaaaaaggccgaggaggccggggccagcgccgccgcctgcgaggccccctccgccgccgggcccggcgcgcccccggagcaggaggcagcccccgcggaggagcccgcggccgccgcagcctcgtcagcctgcgcagccccctcacaggaggcccagcccgagtgcagtccagaagcccccccagcggaggcggcagagtaaaagagcaagcttttgtgagataatcgaagaacttttctcccccgtttgtttgttggagtggtgccaggtactggttttggagaacttgtctacaaccagggattgattttaaagatgtctttttttattttacttttttttaagcaccaaattttgttgttttttttttttctcccctccccacagatcccatctcaaatcattctgttaaccaccattccaacaggtcgaggagagcttaaacaccttcttcctctgccttgtttctcttttattttttattttttcgcatcagtattaatgtttttgcatactttgcatctttattcaaaagtgtaaactttctttgtcaatctatggacatgcccatatatgaaggagatgggtgggtcaaaaagggatatcaaatgaagtgatggggtcacaatggggaaattgaagtggtgcataacattgccaaaatagtgtgccactagaaatggtgtaaaggctgtcttttttttttttttaaaagaaaagttattaccatgtattttgtgaggcaggtttacaacactacaagtcttgagttaagaaggaaagaggaaaaaagaaaaaacaccaatacccagatttaaaaaaaaaaaaacgatcatagtcttaggagttcatttaaaccataggaacttttcacttatctcatgttagctgtaccagtcagtgattaagtagaactacaagttgtataggctttattgtttattgctggtttatgaccttaataaagtgtaattatgtattaccagcagggtgtttttaactgtgactattgtataaaaacaaatcttgatatccagaagcacatgaagtttgcaactttccaccctgcccatttttgtaaaactgcagtcatcttggaccttttaaaacacaaattttaaactcaaccaagctgtgataagtggaatggttactgtttatactgtggtatgtttttgattacagcagataatgctttcttttccagtcgtctttgagaataaaggaaaaaaaatcttcagatgcaatggttttgtgtagcatcttgtctatcatgttttgtaaatactggagaagctttgaccaatttgacttagagatggaatgtaactttgcttacaaaaattgctattaaactcctgcttaaggtgttctaattttctgtgagcacactaaaagcgaaaaataaatgtgaataaaatgtacaaatttgttgtgtttttttatgttctaataatactgagacttctaggtcttaggttaatttttaggaagatcttgcatgccatcaggagtaaattttattgtggttcttaatctgaagttttcaagctctgaaattcataatccgcagtgtcagattacgtagaggaagatcttacaacatttccatgtcaaatctgttaccatttattggcatttagttttcatttaagaattgaacataattatttttattgtagctatatagcatgtcagattaaatcatttacaacaaaaggggtgtgaacctaagactatttaaatgtcttatgagaaaatttcataaagccattctcttgtcattcaggtccagaaacaaattttaaactgagtgagagtctatagaatccatactgcagatgggtcatgaaatgtgaccaaatgtgtttcaaaaattgatggtgtattacctgctattgtaattgcttagtgcttggctaatttccaaattattgcataatatgttctaccttaagaaaacaggtttatgtaacaaagtaatggtgttgaatggatgatgtcagttcatgggcctttagcatagttttaagcatccttttttttttttttttttgaaagtgtgttagcatcttgttactcaaaggataagacagacaataatacttcactgaatcttaataatctttactagtttacctcctctgctctttgccacccgataactggatatcttttccttcaaaggaccctaaactgattgaaatttaagatatgtatcaaaaacattatttcatttaatgcacatctgttttgctgtttttgagcagtgtgcagtttagggttcatgataaatcattgaaccacatgtgtaacaactgaatgccaaatcttaaactcattagaaaaataacaaattaggttttgacacgcattcttaattggaataatggatcaaaaatagtggttcatgaccttaccaaacacccttgctactaataaaatcaaataacacttagaagggtatgtatttttagttagggtttcttgatcttggaggatgtttgaaagttaaaaattgaatttggtaaccaaaggactgatttatgggtctttcctatcttaaccaacgttttcttagttacctagatggccaagtacagtgcctggtatgtagtaagactcagtaaaaaagtggatttttaaaaataactcccaaagtgaatagtcaaaaatcctgttagcaaactgttatatattgctaagtttgttcttttaacagctggaatttattaagatgcattattttgattttattcactgcctaaaacactttgggtggtattgatggagttggtggattttcctccaagtgattaaatgaaatttgacgtatcttttcatccaaagttttgtacatcatgttttctaacggaaaaaaatgttaatatggcttttttgtattactaaaaatagctttgagattaaggaaaaataaataactcttgtacagttcagtattgtctattaaatctgtattggcagtatgtataatggcatttgctgtggttacaaaatacttcctctgggttataataatcatttgatccaattcctattgcttgtaaaataaagttttaccagttgatataatcaa

SEQ ID NO: 16 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 17 - RNA, 24 nt, synthetic construct
  source 1..24: mol_type = other RNA
atgggttgctgtttctccaagacc

SEQ ID NO: 18 - protein, 8 aa, synthetic construct
MGCCFSKT

SEQ ID NO: 19 - RNA, 1225 nt, Homo sapiens; identical to SEQ ID NO: 7
  source 1..1225: mol_type = mRNA

SEQ ID NO: 20 - protein, 228 aa, Homo sapiens; identical to SEQ ID NO: 8

SEQ ID NO: 21 - RNA, 3634 nt, Homo sapiens
  source 1..3634: mol_type = mRNA
agttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatccca

SEQ ID NO: 22 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 23 - RNA, 2432 nt, Homo sapiens
  source 1..2432: mol_type = mRNA
actctcatgttacggcaaaccttaagctgaatgaacaacttttcttctcttgaatatatcttaacgccaaattttgagtgcttttttgttacccatcctcatatgtcccagctagaaagaatcctgggttggagctactgcatgttgattgttttgtttttccttttggctgttcattttggtggctactataaggaaatctaacacaaacagcaactgttttttgttgtttacttttgcatctttacttgtggagctgtggcaagtcctcatatcaaatacagaacatgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttggctgcttcacattttcatccccttctgcatcattgctttcattttcatagccacagtgatagccctaagaaaacaactctgtcaaaagctgtattcttcaaaagacacaacaaaaagacctgtcaccacaacaaagagggaagtgaacagtgctatctgaacctgtggtcttgggagccagggtgacctgatatgacatctaaagaagcttctggactctgaacaagaattcggtggcctgcagagcttgccatttgcacttttcaaatgcctttggatgacccagcactttaatctgaaacctgcaacaagactagccaacacctggccatgaaacttgccccttcactgatctggactcacctctggagcctatggctttaagcaagcactactgcactttacagaattaccccactggatcctggacccacagaattccttcaggatccttcttgctgccagactgaaagcaaaaggaattatttcccctcaagttttctaagtgatttccaaaagcagaggtgtgtggaaatttccagtaacagaaacagatgggttgccaatagagttattttttatctatagcttcctctgggtactagaagaggctattgagactatgagctcacagacagggcttcgcacaaactcaaatcataattgacatgttttatggattactggaatcttgatagcataatgaagttgttctaattaacagagagcatttaaatatacactaagtgcacaaattgtggagtaaagtcatcaagctctgtttttgaggtctaagtcacaaagcatttgttttaacctgtaatggcaccatgtttaatggtggttttttttttgaactacatctttcctttaaaaattattggtttctttttatttgtttttaccttagaaatcaattatatacagtcaaaaatatttgatatgctcatacgttgtatctgcagcaatttcagataagtagctaaaatggccaaagccccaaactaagcctccttttctggccctcaatatgactttaaatttgacttttcagtgcctcagtttgcacatctgtaatacagcaatgctaagtagtcaaggcctttgataattggcactatggaaatcctgcaagatcccactacatatgtgtggagcagaagggtaactcggctacagtaacagcttaattttgttaaatttgttctttatactggagccatgaagctcagagcattagctgacccttgaactattcaaatgggcacattagctagtataacagacttacataggtgggcctaaagcaagctccttaactgagcaaaatttggggcttatgagaatgaaagggtgtgaaattgactaacagacaaatcatacatctcagtttctcaattctcatgtaaatcagagaatgcctttaaagaataaaactcaattgttattcttcaacgttctttatatattctacttttgggta

SEQ ID NO: 24 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 25 - RNA, 1997 nt, Homo sapiens
  source 1..1997: mol_type = mRNA
gctttctattcaagtgccttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttccgga

SEQ ID NO: 26 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 27 - RNA, 1702 nt, Homo sapiens
  source 1..1702: mol_type = mRNA
ataccggcccttcccctcggctttgcctggacagctcctgcctcccgcagggcccacctgtgtcccccagcgccgctccacccagcaggcctgagcccctctctgctgccagacaccccctgctgcccactctcctgctgctcgggttctgaggcacagcttgtcacaccgaggcggattctctttctctttctctttctcttctggcccacagccgcagcaatggcgctgagttcctctgctggagttcatcctgctagctgggttcccgagctgccggtctgagcctgaggcatggagcctcctggagactgggggcctcctccctggagatccacccccaaaaccgacgtcttgaggctggtgctgtatctcaccttcctgggagccccctgctacgccccagctctgccgtcctgcaaggaggacgagtacccagtgggctccgagtgctgccccaagtgcagtccaggttatcgtgtgaaggaggcctgcggggagctgacgggcacagtgtgtgaaccctgccctccaggcacctacattgcccacctcaatggcctaagcaagtgtctgcagtgccaaatgtgtgacccagccatgggcctgcgcgcgagccggaactgctccaggacagagaacgccgtgtgtggctgcagcccaggccacttctgcatcgtccaggacggggaccactgcgccgcgtgccgcgcttacgccacctccagcccgggccagagggtgcagaagggaggcaccgagagtcaggacaccctgtgtcagaactgccccccggggaccttctctcccaatgggaccctggaggaatgtcagcaccagaccaagtgcagctggctggtgacgaaggccggagctgggaccagcagctcccactgggtatggtggtttctctcagggagcctcgtcatcgtcattgtttgctccacagttggcctaatcatatgtgtgaaaagaagaaagccaaggggtgatgtagtcaaggtgatcgtctccgtccagcggaaaagacaggaggcagaaggtgaggccacagtcattgaggccctgcaggcccctccggacgtcaccacggtggccgtggaggagacaataccctcattcacggggaggagcccaaaccactgacccacagactctgcaccccgacgccagagatacctggagcgacggctgctgaaagaggctgtccacctggcggaaccaccggagcccggaggcttgggggctccgccctgggctggcttccgtctcctccagtggagggagaggtggggcccctgctggggtagagctggggacgccacgtgccattcccatgggccagtgagggcctggggcctctgttctgctgtggcctgagctccccagagtcctgaggaggagcgccagttgcccctcgctcacagaccacacacccagccctcctgggccagcccagagggcccttcagaccccagctgtctgcgcgtctgactcttgtggcctcagcaggacaggccccgggcactgcctcacagccaaggctggactgggttggctgcagtgtggtgtttagtggataccacatcggaagtgattttctaaattggatttgaattcggctcctgttttctatttgtcatgaaacagtgtatttggggagatgctgtgggaggatgtaaatatcttgtttctcctcaaa

SEQ ID NO: 28 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 29 - RNA, 1237 nt, Homo sapiens
  source 1..1237: mol_type = mRNA
aatgcagttacaggatcctgggaagcagagtgtctggatggaacctgagctgggtctctgactcacttctgactttagttttttcaagggggaacatggcaaaggtgttcagtttcatccttgttaccaccgctctgacaatgggcagggaaatttcggcgctcgaggactgtgcccaggagcagatgcggctcagagcccaggtgcgcctgcttgagacccgggtcaaacagcaacaggtcaagatcaagcagcttttgcaggagaatgaagtccagttccttgataaaggagatgagaatactgtcattgatcttggaagcaagaggcagtatgcagattgttcagagattttcaatgatgggtataagctcagtggattttacaaaatcaaacctctccagagcccagcagaattttctgtttattgtgacatgtccgatggaggaggatggactgtaattcagagacgatctgatggcagtgaaaactttaacagaggatggaaagactatgaaaatggctttggaaattttgtccaaaaacatggtgaatattggctgggcaataaaaatcttcacttcttgaccactcaagaagactacactttaaaaatcgaccttgcagattttgaaaaaaatagccgttatgcacaatataagaatttcaaagttggagatgaaaagaatttctacgagttgaatattggggaatattctggaacagctggagattcccttgcggggaattttcatcctgaggtgcagtggtgggctagtcaccaaagaatgaaattcagcacgtgggacagagatcatgacaactatgaagggaactgcgcagaagaagatcagtctggctggtggtttaacaggtgtcactctgcaaacctgaatggtgtatactacagcggcccctacacggctaaaacagacaatgggattgtctggtacacctggcatgggtggtggtattctctgaaatctgtggttatgaaaattaggccaaatgattttattccaaatgtaatttaattgctgctgttgggctttcgtttctgcaattcagctttgtttaaagtgatttgaaaaatactcattctgaacatatccatgcgcaatcatgataactgttgtgagtagtgcttttcattcttctcacttgcctttgttacttaatgtgctttcagtacagcagatatgcaatattcaccaaataaatgtagactgtgttaata

SEQ ID NO: 30 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 31 - RNA, 2129 nt, Homo sapiens
  source 1..2129: mol_type = mRNA
agagctccaggcgcacatccgcagtcagccacctcgcgcgcgcctccaggagcaaggatggagaggctggtgatcaggatgcccttctctcatctgtctacctacagcctggtttgggtcatggcagcagtggtgctgtgcacagcacaagtgcaagtggtgacccaggatgaaagagagcagctgtacacacctgcttccttaaaatgctctctgcaaaatgcccaggaagccctcattgtgacatggcagaaaaagaaagctgtaagcccagaaaacatggtcaccttcagcgagaaccatggggtggtgatccagcctgcctataaggacaagataaacattacccagctgggactccaaaactcaaccatcaccttctggaatatcaccctggaggatgaagggtgttacatgtgtctcttcaatacctttggttttgggaagatctcaggaacggcctgcctcaccgtctatgtacagcccatagtatcccttcactacaaattctctgaagaccacctaaatatcacttgctctgccactgcccgcccagcccccatggtcttctggaaggtccctcggtcagggattgaaaatagtacagtgactctgtctcacccaaatgggaccacgtctgttaccagcatcctccatatcaaagaccctaagaatcaggtggggaaggaggtgatctgccaggtgctgcacctggggactgtgaccgactttaagcaaaccgtcaacaaaggctattggttttcagttccgctattgctaagcattgtttccctggtaattcttctcgtcctaatctcaatcttactgtactggaaacgtcaccggaatcaggaccgagagccctaaataagtcacacagcaccctgaaagtgattccctggtctacttgaatttgacacaagagaaaagcaggaggaaaaggggccattctccaaaggacctgaaagagcaaaagaggtgggagcgaaagccttaaggatcccacgactttttactgccatctgagctactcagtgtttgaatcccaagaggaagtcagtttacctctcaggtctgttgtaggacttgattttgtaaagcaatgccatgttatgtggttgaaagggcactggacttagttagtatcaggagcactgagctcacagactgacttgggctcctactggtggggacctctgttagtcactttacctcatccaaagtataaaggaattggaccaaataatttaccacatagctctaaaacttaatttaaaatgtaattccagaaaaaaaaagggaataagcaaagggggaagaattgaaagagagagagaagaaagaatacagagagcttaccttttgcctttctgttgatgttacatctcttcttcctatgttcttaggtctatgagtctgtttccccatcatttggtatctagtccagttcctgcttactgctttgctaatagctggccttgctagaatccttggtttcactgctgttcttcatgtgcttctatgagatttactccaacacaaataggactgaatttattgtgaagtaacattggcaatcttaacttattcatttaacttatttttatagctagataaatattgttagtcttagacaatagctcacattttttgagaagcatgccctccctgtccatttgtcttataacatgacccagccctattttacgtcattctaaattcagcctcatataatgaaaatacattatgaaaacagatgtttaggagatttcctgtatagcagtcagccaattcatatgctttgtctctgctggcttctttttccatgcgttaacttttcccaatagcagaggaggcaaatatgagcatacaatccctttgttctaaagatattgttccagctagtggaatgatgttgaatctttaataaccataattagttgctttttcagtatcttctgctttgtctgtgtctatccagtggcctaggaattaaagtgtaagttgttttcgctgttaaattggatatttatatatatatatagcaagattttcatgtgttatttaattctgtattgtttcttatatttgtagtaaaatattgaacaattaaaagtgttgactccaaa

SEQ ID NO: 32 - protein, 269 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDREP

SEQ ID NO: 33 - RNA, 1724 nt, Homo sapiens
  source 1..1724: mol_type = mRNA
ctttgttaagtcgttccctctacaaaggacttcctagtgggtgtgaaaggcagcggtggccacagaggcggcggagagatggccttcagcggttcccaggctccctacctgagtccagctgtccccttttctgggactattcaaggaggtctccaggacggacttcagatcactgtcaatgggaccgttctcagctccagtggaaccaggtttgctgtgaactttcagactggcttcagtggaaatgacattgccttccacttcaaccctcggtttgaagatggagggtacgtggtgtgcaacacgaggcagaacggaagctgggggcccgaggagaggaagacacacatgcctttccagaaggggatgccctttgacctctgcttcctggtgcagagctcagatttcaaggtgatggtgaacgggatcctcttcgtgcagtacttccaccgcgtgcccttccaccgtgtggacaccatctccgtcaatggctctgtgcagctgtcctacatcagcttccagaacccccgcacagtccctgttcagcctgccttctccacggtgccgttctcccagcctgtctgtttcccacccaggcccagggggcgcagacaaaaacctcccggcgtgtggcctgccaacccggctcccattacccagacagtcatccacacagtgcagagcgcccctggacagatgttctctactcccgccatcccacctatgatgtacccccaccccgcctatccgatgcctttcatcaccaccattctgggagggctgtacccatccaagtccatcctcctgtcaggcactgtcctgcccagtgctcagaggttccacatcaacctgtgctctgggaaccacatcgccttccacctgaacccccgttttgatgagaatgctgtggtccgcaacacccagatcgacaactcctgggggtctgaggagcgaagtctgccccgaaaaatgcccttcgtccgtggccagagcttctcagtgtggatcttgtgtgaagctcactgcctcaaggtggccgtggatggtcagcacctgtttgaatactaccatcgcctgaggaacctgcccaccatcaacagactggaagtggggggcgacatccagctgacccatgtgcagacataggcggcttcctggccctggggccgggggctggggtgtggggcagtctgggtcctctcatcatccccacttcccaggcccagcctttccaaccctgcctgggatctgggctttaatgcagaggccatgtccttgtctggtcctgcttctggctacagccaccctggaacggagaaggcagctgacggggattgccttcctcagccgcagcagcacctggggctccagctgctggaatcctaccatcccaggaggcaggcacagccagggagaggggaggagtgggcagtgaagatgaagccccatgctcagtcccctcccatcccccacgcagctccaccccagtcccaagccaccagctgtctgctcctggtgggaggtggcctcctcagcccctcctctctgacctttaacctcactctcaccttgcaccgtgcaccaacccttcacccctcctggaaagcaggcctgatggcttcccactggcctccaccacctgaccagagtgttctcttcagaggactggctcctttcccagtgtccttaaaataaagaaatgaaaatgcttgttggcacattca

SEQ ID NO: 34 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 35 - RNA, 5792 nt, Homo sapiens
  source 1..5792: mol_type = mRNA
agtcacttgtctggagcttgaagaagtgggtattccccttcccaccccaggcactggaggagcggccccccggggattccaggacctgagctccgggagctggactcgcagcgaccgcggcagagcgagcgggcgccgggaagcgaggagacgcccgcgggaggcccagctgctcggagcaactggcatggcccgagccatggccgccgcgtggccgctgctgctggtggcgctactggtgctgtcctggccacccccaggaaccggggacgtcgtcgtgcaggcgcccacccaggtgcccggcttcttgggcgactccgtgacgctgccctgctacctacaggtgcccaacatggaggtgacgcatgtgtcacagctgacttgggcgcggcatggtgaatctggcagcatggccgtcttccaccaaacgcagggccccagctattcggagtccaaacggctggaattcgtggcagccagactgggcgcggagctgcggaatgcctcgctgaggatgttcgggttgcgcgtagaggatgaaggcaactacacctgcctgttcgtcacgttcccgcagggcagcaggagcgtggatatctggctccgagtgcttgccaagccccagaacacagctgaggttcagaaggtccagctcactggagagccagtgcccatggcccgctgcgtctccacagggggtcgcccgccagcccaaatcacctggcactcagacctgggcgggatgcccaatacgagccaggtgccagggttcctgtctggcacagtcactgtcaccagcctctggatattggtgccctcaagccaggtggacggcaagaatgtgacctgcaaggtggagcacgagagctttgagaagcctcagctgctgactgtgaacctcaccgtgtactaccccccagaggtatccatctctggctatgataacaactggtaccttggccagaatgaggccaccctgacctgcgatgctcgcagcaacccagagcccacaggctataattggagcacgaccatgggtcccctgccaccctttgctgtggcccagggcgcccagctcctgatccgtcctgtggacaaaccaatcaacacaactttaatctgcaacgtcaccaatgccctaggagctcgccaggcagaactgaccgtccaggtcaaagagggacctcccagtgagcactcaggcatgtcccgtaacgccatcatcttcctggttctgggaatcctggtttttctgatcctgctggggatcgggatttatttctattggtccaaatgttcccgtgaggtcctttggcactgtcatctgtgtccctcgagtacagagcatgccagcgcctcagctaatgggcatgtctcctattcagctgtgagcagagagaacagctcttcccaggatccacagacagagggcacaaggtgacagcgtcgggactgagaggggagagagactggagctggcaaggacgtgggcctccagagttggacccgaccccaatggatgaagaccccctccaaagagaccagcctccctccctgtgccagacctcaaaacgacgggggcaggtgcaagttcataggtctccaagaccaccctcctttcatttgctagaaggactcactagactcaggaaagctgttaggctcacagttacagtttattacagtaaaaggacagagattaagatcagcaaagggaggaggtgcacagcacacgttccacgacagatgaggcgacggcttccatctgccctctcccagtggagccatataggcagcacctgattctcacagcaacatgtgacaacatgcaagaagtactgccaatactgccaaccagagcagctcactcgagatctttgtgtccagagttttttgtttgtcttgagacagggtctggctctgttggcagactagagtacagtggtgagatcacagttcattgcagccttgacttctcaacgccaagtcatcctcccacctcagcctcctgagtagctatgactacaggtatgtgccaccacgtctggctaatctttttattatttgtaaagtcgaggtttccctgtgttgcccaggctggtcttgaactcttggctccaagtgatacttctgccttggcctcccaaagtgctgaattaagcagctcaccatccacacggctgacctcatacatcaagccaataccgtgtggcccaagacccccaccataaatcacatcattagcatgaaccacccagagtggcccaagactccaagatcagctaccaggcaggatattccaagggcttagagatgaatgcccaggagctgaggataaagggcccgatctttctttgggcaaggttaagcctttactgcatagcagaccacacagaagggtgtgggccaccagagaattttggtaaaaatttggcctctggccttgagcttctaaatctctgtatccgtcagatctctgtggttacaagaaacagccactgaccctggtcaccagaggctgcaattcaggccgcaagcagctgcctggggggtgtccaaggagcagagaaaactactagatgtgaacttgaagaaggttgtcagctgcagccactttctgccagcatctgcagccactttctgccagcatctgcagccagcaagctgggactggcaggaaataacccacaaaagaagcaaatgcaatttccaacacaagggggaagggatgcagggggaggcagcgctgcagttgctcaggacacgctcctataggaccaagatggatgcgacccaagacccaggaggcccagctgctcagtgcaactgacaagttaaaaaggtctatgatcttgagggcagacagcagaattcctcttataaagaaaactgtttgggaaaatacgttgagggagagaagaccttgggccaagatgctaaatgggaatgcaaagcttgagctgctctgcaagagaaaataagcaggacagaggatttgctctggacagagatggaagagccgggaacagagaagtgtggggaagagataggaaccagcaggatggcaggggcaaagggctcaagggtgaggaggccagtgggaccccacagagttggggagataaaggaacattggttgctttggtggcacgtaagctccttgtctgtctccagcacccagaatctcattaaagcttatttattgtacctccagcggctgtgtgcaatggggtcttttgtggaaatcaaggagcagacaggtttcatgtgtactgtcaccacgtgggatggaaccagaggcatggaagcaagacgctaaatgaagagggccataagggctgggattcccaggcaccttaggaacagcttgtctttttttttttcctctccaaaaaaaatgtttaagggacggtgtctcctgtcacccaggctggagtgcaatggcacgatcatagctcattgcagcctctaactccggggctcaagcaatcctcccacctcagcctaccaagtagctgtgaccacagctgcccctcaccatgctaagctaatttttttaattagatagtacataaacgtcccaaaattagaagataaaaagacatgagggatccattctaatttgtgtttggagtgtaatggtccagctccattcttctgcacatggatatccagttttacacaacactgtgaatgtaatgaatgccactgaatcatacactcaaaaatagctaaaatggcaaattgtctgttatctctttttaaccaccatttttgaaaattaattataccaaaaaaccattgaatagtgcactttatttatttatttatttgtttatttatttatttattttagaaataagagtctcactttgttgcccaggctggagtgcagtggcgtgatcatggctcattgcagcctcgacctgctgggctcgggctatccttccatctcagcctcccgagtagctgggactataggtgggcgccaccccacctggctaaatctctttttaacttttgtagagataggcatctcgctatgttgcctaggctgggctggaactcctgggctcaagtgctcctcctgccttggcctcccaaagcgctaggattacagatgtgagccaccgcgcccaccctgaaccttactttttttgctcagtttctggtaattcagagaatgcctcctgagttgttctacacccacctcatattccatgggagggctgtacagggcttttttaacgaggcctctaaggacaggcatttgtatcctttccagcctttcactattacaatgttgtagtgaataactttacacactgtcatttattttacttttttttttttttattttagagaaaggaatcttgccatcttgcccaggctggtctcaaattcctgggcccaaacaatcctcccgccttggcctcctaaagtactgggatttataggcataagccaccgtgcctggccaatgcacactgtcatttagctcatgttaacacctgagtgtaggacacactcctggaggtggaattgctgggccaaagagtatgtttcttgtcattgtgatagatattgacaaatgaaccctcacagaagttgtgctgagttctgttcccaccagcgacgtaggcgatgacctttttctggagggagggggcatccttggagtccacagagccaggaatggagagtgggcccagaattttggtataggtgttgtataaacttatagtaaggttaagaaaaccgcaactatccttatcagagacttggcggggggcagggtatgatggagatcataaggaggctaaaacactccacaccctccctctgcattgctcctgcacgggagtcgggaatcttttcaggttgatacgatctcaccttgaggagctgtgaggtcccagaagcctctgggttgcagattgcttggggtgaaaatgtctgtgctactgaaatctaactttttacaaaaaattacgggctgggcgcagtggctcacgcctgtaatcccagcactttgggaggctgcagcgggtggatcacttgaggtaaggagttcaagaccagaccatagtgaaaccgtgtctctacaaaaaaaattagccaggtgtggtggtgcatgcttgtaatcccagctactcagaaggctgaggtgggagaatcccttgaacccgggaagtggaggctggagtaaaccatgatcgagttactgcactccagcctgggtgacaagagtgagactctgtctccaaaaaaaaaaaaaaaaaaaaaaaaactggattgcctggctctactccgggcacagcatgcaggcccagttctgctgctctgctgtttgttctgctttcctccacatattggcatcaccctctggtgccaagatggctgctgcattccaggcatcacatccagactcagacccagagaagctgcccatccctacctgggtgagcctttgtaggaacgagaaaccgcatccagcagcagaaacctcacccagcagcgtcttttccggtctcattcaccagcgccgcccaccgctcaaccaatccctggccaaaagaatgggaccgcctggaaggctggaccaaacaggacctgccctctggggctggggagaggcccagatgaaggctgcaggacaggatggactcctagacctctgttaccagcagtgactacctctgtctgggtggttggaacatgtttgaattttattctaagtactgtctacaagttctgcaataaaccttgactcttcttttaataatgcaaaa

SEQ ID NO: 36 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGMSRNAIIFLVLGILVFLILLGIGIYFYWSKCSREVLWHCHLCPSSTEHASASANGHVSYSAVSRENSSSQDPQTEGTR

SEQ ID NO: 37 - RNA, 1987 nt, Homo sapiens
  source 1..1987: mol_type = mRNA
gtgacgtcagcgggttcgaaccgccggagctgagcgagaggccgggggtgccgagccgggcggggagagctgggccgggagagcagaacagggaggctagagcgcagcgggaaccggcccggagccggagccggagccccacaggcacctactaaaccgcccagccgatcggcccccacagagtggcccgcgggcctccggccgggcccagtcccctcccgggccctccatggcccgggccgctgccctcctgccgtcgagatcgccgccgacgccgctgctgtggccgctgctgctgctgctgctcctggaaaccggagcccaggatgtgcgagttcaagtgctacccgaggtgcgaggccagctcgggggcaccgtggagctgccgtgccacctgctgccacctgttcctggactgtacatctccctggtgacctggcagcgcccagatgcacctgcgaaccaccagaatgtggccgccttccaccctaagatgggtcccagcttccccagcccgaagcctggcagcgagcggctgtccttcgtctctgccaagcagagcactgggcaagacacagaggcagagctccaggacgccacgctggccctccacgggctcacggtggaggacgagggcaactacacttgcgagtttgccaccttccccaaggggtccgtccgagggatgacctggctcagagtcatagccaagcccaagaaccaagctgaggcccagaaggtcacgttcagccaggaccctacgacagtggccctctgcatctccaaagagggccgcccacctgcccggatctcctggctctcatccctggactgggaagccaaagagactcaggtgtcagggaccctggccggaactgtcactgtcaccagccgcttcaccttggtgccctcgggccgagcagatggtgtcacggtcacctgcaaagtggagcatgagagcttcgaggaaccagccctgatacctgtgaccctctctgtacgctaccctcctgaagtgtccatctccggctatgatgacaactggtacctcggccgtactgatgccaccctgagctgtgacgtccgcagcaacccagagcccacgggctatgactggagcacgacctcaggcaccttcccgacctccgcagtggcccagggctcccagctggtcatccacgcagtggacagtctgttcaataccaccttcgtctgcacagtcaccaatgccgtgggcatgggccgcgctgagcaggtcatctttgtccgagaaacccccagggcctcgccccgagatgtgggcccgctggtgtggggggccgtgggggggacactgctggtgctgctgcttctggctggggggtccttggccttcatcctgctgagggtgaggaggaggaggaagagccctggaggagcaggaggaggagccagtggcgacgggggattctacgatccgaaagctcaggtgttgggaaatggggaccccgtcttctggacaccagtagtccctggtcccatggaaccagatggcaaggatgaggaggaggaggaggaggaagagaaggcagagaaaggcctcatgttgcctccacccccagcactcgaggatgacatggagtcccagctggacggctccctcatctcacggcgggcagtttatgtgtgacctggacacagacagagacagagccaggcccggccctcccgcccccgacctgaccacgccggcctagggttccagactggttggacttgttcgtctggacgacactggagtggaacactgcctcccactttcttgggacttggagggaggtggaacagcacactggacttctcccgtctctagggctgcatggggagcccggggagctgagtagtggggatccagagaggacccccgcccccagagacttggttttggctccagccttcccctggccccgtgacactcaggagttaataaatgccttggaggaaaaca

SEQ ID NO: 38 - protein, 479 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPRASPRDVGPLVWGAVGGTLLVLLLLAGGSLAFILLRVRRRRKSPGGAGGGASGDGGFYDPKAQVLGNGDPVFWTPVVPGPMEPDGKDEEEEEEEEKAEKGLMLPPPPALEDDMESQLDGSLISRRAVYV

SEQ ID NO: 39 - RNA, 2690 nt, Homo sapiens
  source 1..2690: mol_type = mRNA
gtgacgtcagcgggttcgaaccgccggagctgagcgagaggccgggggtgccgagccgggcggggagagctgggccgggagagcagaacagggaggctagagcgcagcgggaaccggcccggagccggagccggagccccacaggcacctactaaaccgcccagccgatcggcccccacagagtggcccgcgggcctccggccgggcccagtcccctcccgggccctccatggcccgggccgctgccctcctgccgtcgagatcgccgccgacgccgctgctgtggccgctgctgctgctgctgctcctggaaaccggagcccaggatgtgcgagttcaagtgctacccgaggtgcgaggccagctcgggggcaccgtggagctgccgtgccacctgctgccacctgttcctggactgtacatctccctggtgacctggcagcgcccagatgcacctgcgaaccaccagaatgtggccgccttccaccctaagatgggtcccagcttccccagcccgaagcctggcagcgagcggctgtccttcgtctctgccaagcagagcactgggcaagacacagaggcagagctccaggacgccacgctggccctccacgggctcacggtggaggacgagggcaactacacttgcgagtttgccaccttccccaaggggtccgtccgagggatgacctggctcagagtcatagccaagcccaagaaccaagctgaggcccagaaggtcacgttcagccaggaccctacgacagtggccctctgcatctccaaagagggccgcccacctgcccggatctcctggctctcatccctggactgggaagccaaagagactcaggtgtcagggaccctggccggaactgtcactgtcaccagccgcttcaccttggtgccctcgggccgagcagatggtgtcacggtcacctgcaaagtggagcatgagagcttcgaggaaccagccctgatacctgtgaccctctctgtacgctaccctcctgaagtgtccatctccggctatgatgacaactggtacctcggccgtactgatgccaccctgagctgtgacgtccgcagcaacccagagcccacgggctatgactggagcacgacctcaggcaccttcccgacctccgcagtggcccagggctcccagctggtcatccacgcagtggacagtctgttcaataccaccttcgtctgcacagtcaccaatgccgtgggcatgggccgcgctgagcaggtcatctttgtccgagagacccccaacacagcaggcgcaggggccacaggcggcatcatcgggggcatcatcgccgccatcattgctactgctgtggctgccacgggcatccttatctgccggcagcagcggaaggagcagacgctgcagggggcagaggaggacgaagacctggagggacctccctcctacaagccaccgaccccaaaagcgaagctggaggcacaggagatgccctcccagctcttcactctgggggcctcggagcacagcccactcaagaccccctactttgatgctggcgcctcatgcactgagcaggaaatgcctcgataccatgagctgcccaccttggaagaacggtcaggacccttgcaccctggagccacaagcctggggtcccccatcccggtgcctccagggccacctgctgtggaagacgtttccctggatctagaggatgaggagggggaggaggaggaagagtatctggacaagatcaaccccatctatgatgctctgtcctatagcagcccctctgattcctaccagggcaaaggctttgtcatgtcccgggccatgtatgtgtgagctgccatgcgcctggcgtctcacatctcacctgttgatcccttagctttcttgccaaggatctagtgccccctgacctctggccaggccactgtcagttaacacatatgcattccatttgtgatgtctaccttggtggctccactatgacccctaacccatgagcccagagaaattcaccgtgataatggaatcctggcaaccttatctcatgaggcaggaggtggggaaggtgcttctgcacaacctctgatcccaaggactcctctcccagactgtgaccttagaccatacctctcaccccccaatgcctcgactcccccaaaatcacaaagaagaccctagacctataatttgtcttcaggtagtaaattcccaataggtctgctggagtgggcgctgagggctccctgctgctcagacctgagccctccaggcagcagggtcccacttaccccctccccaccctgttccccaaaggtgggaaagaggggattccccagcccaaggcagggttttcccagcaccctcctgtaagcagaagtctcagggtccagacccttccctgagcccccacccccaccccaattcctgcctaccaagcaagcagccccagcctagggtcagacagggtgagcctcatacagactgtgccttgatggccccagccttgggagaagaatttactgttaacctggaagactactgaatcattttacccttgcccagtggaataggacctaaacatcccccttccggggaaagtgggtcatctgaattgggggtagcaattgatactgttttgtaaactacatttcctacaaaatatgaatttatactttga

SEQ ID NO: 40 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 41 - RNA, 1630 nt, Homo sapiens
  source 1..1630: mol_type = mRNA
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatca

SEQ ID NO: 42 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 43 - RNA, 1439 nt, Homo sapiens
  source 1..1439: mol_type = mRNA
agtcacatttcagccactgctctgagaatttgtgagcagcccctaacaggctgttacttcactacaactgacgatatgatcatcttaatttacttatttctcttgctatgggaagacactcaaggatggggattcaaggatggaatttttcataactccatatggcttgaacgagcagccggtgtgtaccacagagaagcacggtctggcaaatacaagctcacctacgcagaagctaaggcggtgtgtgaatttgaaggcggccatctcgcaacttacaagcagctagaggcagccagaaaaattggatttcatgtctgtgctgctggatggatggctaagggcagagttggataccccattgtgaagccagggcccaactgtggatttggaaaaactggcattattgattatggaatccgtctcaataggagtgaaagatgggatgcctattgctacaacccacacgcaaaggagtgtggtggcgtctttacagatccaaagcaaatttttaaatctccaggcttcccaaatgagtacgaagataaccaaatctgctactggcacattagactcaagtatggtcagcgtattcacctgagttttttagattttgaccttgaagatgacccaggttgcttggctgattatgttgaaatatatgacagttacgatgatgtccatggctttgtgggaagatactgtggagatgagcttccagatgacatcatcagtacaggaaatgtcatgaccttgaagtttctaagtgatgcttcagtgacagctggaggtttccaaatcaaatatgttgcaatggatcctgtatccaaatccagtcaaggaaaaaatacaagtactacttctactggaaataaaaactttttagctggaagatttagccacttataaaaaaaaaaaaaaggatgatcaaaacacacagtgtttatgttggaatcttttggaactcctttgatctcactgttattattaacatttatttattatttttctaaatgtgaaagcaatacataatttagggaaaattggaaaatataggaaactttaaacgagaaaatgaaacctctcataatcccactgcatagaaataacaagcgttaacattttcatatttttttctttcagtcatttttctatttgtggtatatgtatatatgtacctatatgtatttgcatttgaaattttggaatcctgctctatgtacagttttgtattatactttttaaatcttgaactttataaacattttctgaaatcattgattattctacaaaaacatgattttaaacagctgtaaaatattctatgatatgaatgttttatgcattatttaagcctgtctctattgttggaatttcaggtcattttcataaatattgttgcaataaatatccttgaacacacaaaaaaaaaaaaaaaa

SEQ ID NO: 44 - protein, 277 aa, Homo sapiens
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 45 - RNA, 3295 nt, Homo sapiens
  source 1..3295: mol_type = mRNA
attcgggccgggcctcgctgcggcggcgactgagccaggctgggccgcgtccctgagtcccagagtcggcgcggcgcggcaggggcagccttccaccacggggagcccagctgtcagccgcctcacaggaagatgctgcgtcggcggggcagccctggcatgggtgtgcatgtgggtgcagccctgggagcactgtggttctgcctcacaggagccctggaggtccaggtccctgaagacccagtggtggcactggtgggcaccgatgccaccctgtgctgctccttctcccctgagcctggcttcagcctggcacagctcaacctcatctggcagctgacagataccaaacagctggtgcacagctttgctgagggccaggaccagggcagcgcctatgccaaccgcacggccctcttcccggacctgctggcacagggcaacgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccagggctaccctgaggctgaggtgttctggcaggatgggcagggtgtgcccctgactggcaacgtgaccacgtcgcagatggccaacgagcagggcttgtttgatgtgcacagcatcctgcgggtggtgctgggtgcaaatggcacctacagctgcctggtgcgcaaccccgtgctgcagcaggatgcgcacagctctgtcaccatcacaccccagagaagccccacaggagccgtggaggtccaggtccctgaggacccggtggtggccctagtgggcaccgatgccaccctgcgctgctccttctcccccgagcctggcttcagcctggcacagctcaacctcatctggcagctgacagacaccaaacagctggtgcacagtttcaccgaaggccgggaccagggcagcgcctatgccaaccgcacggccctcttcccggacctgctggcacaaggcaatgcatccctgaggctgcagcgcgtgcgtgtggcggacgagggcagcttcacctgcttcgtgagcatccgggatttcggcagcgctgccgtcagcctgcaggtggccgctccctactcgaagcccagcatgaccctggagcccaacaaggacctgcggccaggggacacggtgaccatcacgtgctccagctaccggggctaccctgaggctgaggtgttctggcaggatgggcagggtgtgcccctgactggcaacgtgaccacgtcgcagatggccaacgagcagggcttgtttgatgtgcacagcgtcctgcgggtggtgctgggtgcgaatggcacctacagctgcctggtgcgcaaccccgtgctgcagcaggatgcgcacggctctgtcaccatcacagggcagcctatgacattccccccagaggccctgtgggtgaccgtggggctgtctgtctgtctcattgcactgctggtggccctggctttcgtgtgctggagaaagatcaaacagagctgtgaggaggagaatgcaggagctgaggaccaggatggggagggagaaggctccaagacagccctgcagcctctgaaacactctgacagcaaagaagatgatggacaagaaatagcctgaccatgaggaccagggagctgctacccctccctacagctcctaccctctggctgcaatggggctgcactgtgagccctgcccccaacagatgcatcctgctctgacaggtgggctccttctccaaaggatgcgatacacagaccactgtgcagccttatttctccaatggacatgattcccaagtcatcctgctgccttttttcttatagacacaatgaacagaccacccacaaccttagttctctaagtcatcctgcctgctgccttatttcacagtacatacatttcttagggacacagtacactgaccacatcaccaccctcttcttccagtgctgcgtggaccatctggctgccttttttctccaaaagatgcaatattcagactgactgaccccctgccttatttcaccaaagacacgatgcatagtcaccccggccttgtttctccaatggccgtgatacactagtgatcatgttcagccctgcttccacctgcatagaatcttttcttctcagacagggacagtgcggcctcaacatctcctggagtctagaagctgtttcctttcccctccttcctcctcttgctctagccttaatactggccttttccctccctgccccaagtgaagacagggcactctgcgcccaccacatgcacagctgtgcatggagacctgcaggtgcacgtgctggaacacgtgtggttcccccctggcccagcctcctctgcagtgcccctctcccctgcccatcctccccacggaagcatgtgctggtcacactggttctccaggggtctgtgatggggcccctgggggtcagcttctgtccctctgccttctcacctctttgttcctttcttttcatgtatccattcagttgatgtttattgagcaactacagatgtcagcactgtgttaggtgctgggggccctgcgtgggaagataaagttcctccctcaaggactccccatccagctgggagacagacaactaactacactgcaccctgcggtttgcagggggctcctgcctggctccctgctccacacctcctctgtggctcaaggcttcctggatacctcacccccatcccacccataattcttacccagagcatggggttggggcggaaacctggagagagggacatagcccctcgccacggctagagaatctggtggtgtccaaaatgtctgtccaggtgtgggcaggtgggcaggcaccaaggccctctggacctttcatagcagcagaaaaggcagagcctggggcagggcagggccaggaatgctttggggacaccgaggggactgccccccacccccaccatggtgctattctggggctggggcagtcttttcctggcttgcctctggccagctcctggcctctggtagagtgagacttcagacgttctgatgccttccggatgtcatctctccctgccccaggaatggaagatgtgaggacttctaatttaaatgtgggactcggagggattttgtaaactgggggtatattttggggaaaataaatgtctttgtaaaaa

SEQ ID NO: 46 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 47 - RNA, 2605 nt, Homo sapiens
  source 1..2605: mol_type = mRNA
gtgagtcaccaaggaaggcagcggcagctccactcagccagtacccagatacgctgggaaccttccccagccatggcttccctggggcagatcctcttctggagcataattagcatcatcattattctggctggagcaattgcactcatcattggctttggtatttcagggagacactccatcacagtcactactgtcgcctcagctgggaacattggggaggatggaatcctgagctgcacttttgaacctgacatcaaactttctgatatcgtgatacaatggctgaaggaaggtgttttaggcttggtccatgagttcaaagaaggcaaagatgagctgtcggagcaggatgaaatgttcagaggccggacagcagtgtttgctgatcaagtgatagttggcaatgcctctttgcggctgaaaaacgtgcaactcacagatgctggcacctacaaatgttatatcatcacttctaaaggcaaggggaatgctaaccttgagtataaaactggagccttcagcatgccggaagtgaatgtggactataatgccagctcagagaccttgcggtgtgaggctccccgatggttcccccagcccacagtggtctgggcatcccaagttgaccagggagccaacttctcggaagtctccaataccagctttgagctgaactctgagaatgtgaccatgaaggttgtgtctgtgctctacaatgttacgatcaacaacacatactcctgtatgattgaaaatgacattgccaaagcaacaggggatatcaaagtgacagaatcggagatcaaaaggcggagtcacctacagctgctaaactcaaaggcttctctgtgtgtctcttctttctttgccatcagctgggcacttctgcctctcagcccttacctgatgctaaaataatgtgcctcggccacaaaaaagcatgcaaagtcattgttacaacagggatctacagaactatttcaccaccagatatgacctagttttatatttctgggaggaaatgaattcatatctagaagtctggagtgagcaaacaagagcaagaaacaaaaagaagccaaaagcagaaggctccaatatgaacaagataaatctatcttcaaagacatattagaagttgggaaaataattcatgtgaactagacaagtgtgttaagagtgataagtaaaatgcacgtggagacaagtgcatccccagatctcagggacctccccctgcctgtcacctggggagtgagaggacaggatagtgcatgttctttgtctctgaatttttagttatatgtgctgtaatgttgctctgaggaagcccctggaaagtctatcccaacatatccacatcttatattccacaaattaagctgtagtatgtaccctaagacgctgctaattgactgccacttcgcaactcaggggcggctgcattttagtaatgggtcaaatgattcactttttatgatgcttccaaaggtgccttggcttctcttcccaactgacaaatgccaaagttgagaaaaatgatcataattttagcataaacagagcagtcggcgacaccgattttataaataaactgagcaccttctttttaaacaaacaaatgcgggtttatttctcagatgatgttcatccgtgaatggtccagggaaggacctttcaccttgtctatatggcattatgtcatcacaagctctgaggcttctcctttccatcctgcgtggacagctaagacctcagttttcaatagcatctagagcagtgggactcagctggggtgatttcgccccccatctccgggggaatgtctgaagacaattttggttacctcaatgagggagtggaggaggatacagtgctactaccaactagtggatagaggccagggatgctgctcaacctcctaccatgtacaggacgtctccccattacaactacccaatccgaagtgtcaactgtgtcagggctaagaaaccctggttttgagtagaaaagggcctggaaagaggggagccaacaaatctgtctgcttcctcacattagtcattggcaaataagcattctgtctctttggctgctgcctcagcacagagagccagaactctatcgggcaccaggataacatctctcagtgaacagagttgacaaggcctatgggaaatgcctgatgggattatcttcagcttgttgagcttctaagtttctttcccttcattctaccctgcaagccaagttctgtaagagaaatgcctgagttctagctcaggttttcttactctgaatttagatctccagaccctgcctggccacaattcaaattaaggcaacaaacatataccttccatgaagcacacacagacttttgaaagcaaggacaatgactgcttgaattgaggccttgaggaatgaagctttgaaggaaaagaatactttgtttccagcccccttcccacactcttcatgtgttaaccactgccttcctggaccttggagccacggtgactgtattacatgttgttatagaaaactgattttagagttctgatcgttcaagagaatgattaaatatacatttcctacacca

SEQ ID NO: 48 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 49 - RNA, 4714 nt, Homo sapiens
  source 1..4714: mol_type = mRNA
agtcgcgggaggcttccccgcgccggccgcgtcccgcccgctccccggcaccagaagttcctctgcgcgtccgacggcgacatgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctagcccagctgggggacagtgggctgttgtggctgggtctggggcaggtgcatttgagccagggctggctctgtgagtggcctccttggcctcggccctggttccctccctcctgctctgggctcagatactgtgacatcccagaagcccagcccctcaacccctctggatgctacatggggatgctggacggctcagcccctgttccaaggattttggggtgctgagattctcccctagagacctgaaattcaccagctacagatgccaaatgacttacatcttaagaagtctcagaacgtccagcccttcagcagctctcgttctgagacatgagccttgggatgtggcagcatcagtgggacaagatggacactgggccaccctcccaggcaccagacacagggcacggtggagagacttctcccccgtggccgccttggctcccccgttttgcccgaggctgctcttctgtcagacttcctctttgtaccacagtggctctggggccaggcctgcctgcccactggccatcgccaccttccccagctgcctcctaccagcagtttctctgaagatctgtcaacaggttaagtcaatctggggcttccactgcctgcattccagtccccagagcttggtggtcccgaaacgggaagtacatattggggcatggtggcctccgtgagcaaatggtgtcttgggcaatctgaggccaggacagatgttgccccacccactggagatggtgctgagggaggtgggtggggccttctgggaaggtgagtggagaggggcacctgccccccgccctccccatcccctactcccactgctcagcgcgggccattgcaagggtgccacacaatgtcttgtccaccctgggacacttctgagtatgaagcgggatgctattaaaaactacatggggaaacaggtgcaaaccctggagatggattgtaagagccagtttaaatctgcactctgctgctcctcccccacccccaccttccactccatacaatctgggcctggtggagtcttcgcttcagagccattcggccaggtgcgggtgatgttcccatctcctgcttgtgggcatgccctggctttgtttttatacacataggcaaggtgagtcctctgtggaattgtgattgaaggattttaaagcaggggaggagagtagggggcatctctgtacactctgggggtaaaacagggaaggcagtgcctgagcatggggacaggtgaggtggggctgggcagaccccctgtagcgtttagcaggatgggggccccaggtactgtggagagcatagtccagcctgggcatttgtctcctagcagcctacactggctctgctgagctgggcctgggtgctgaaagccaggatttggggctaggcgggaagatgttcgcccaattgcttggggggttggggggatggaaaaggggagcacctctaggctgcctggcagcagtgagccctgggcctgtggctacagccagggaaccccacctggacacatggccctgcttctaagccccccagttaggcccaaaggaatggtccactgagggcctcctgctctgcctgggctgggccaggggctttgaggagagggtaaacataggcccggagatggggctgacacctcgagtggccagaatatgcccaaaccccggcttctcccttgtccctaggcagaggggggtcccttcttttgttccctctggtcaccacaatgcttgatgccagctgccataggaagagggtgctggctggccatggtggcacacacctgtcctcccagcactttgcagggctgaggtggaaggaccgcttaagcccaggtgttcaaggctgctgtgagctgtgttcgagccactacactccagcctggggacggagcaaaactttgcctcaaaacaaattttaaaaagaaagaaagaaggaaagagggtatgtttttcacaattcatgggggcctgcatggcaggagtggggacaggacacctgctgttcctggagtcgaaggacaagcccacagcccagattccggttctcccaactcaggaagagcatgccctgccctctggggaggctggcctggccccagccctcagctgctgaccttgaggcagagacaacttctaagaatttggctgccagaccccaggcctggctgctgctgtgtggagagggaggcggcccgcagcagaacagccaccgcacttcctcctcagcttcctctggtgcggccctgccctctcttctctggacccttttacaactgaacgcatctgggcttcgtggtttcctgttttcagcgaaatttactctgagctcccagttccatcttcatccatggccacaggccctgcctacaacgcactagggacgtccctccctgctgctgctggggaggggcaggctgctggagccgccctctgagttgcccgggatggtagtgcctctgatgccagccctggtggctgtgggctggggtgcatgggagagctgggtgcgagaacatggcgcctccagggggcgggaggagcactaggggctggggcaggaggctcctggagcgctggattcgtggcacagtctgaggccctgagagggaaatccatgcttttaagaactaattcattgttaggagatcaatcaggaattaggggccatcttacctatctcctgacattcacagtttaatagagacttcctgcctttattccctcccagggagaggctgaaggaatggaattgaaagcaccatttggagggttttgctgacacagcggggactgctcagcactccctaaaaacacaccatggaggccactggtgactgctggtgggcaggctggccctgcctgggggagtccgtggcgatgggcgctggggtggaggtgcaggagccccaggacctgcttttcaaaagacttctgcctgaccagagctcccactacatgcagtggcccagggcagaggggctgatacatggcctttttcagggggtgctcctcgcggggtggacttgggagtgtgcagtgggacagggggctgcaggggtcctgccaccaccgagcaccaacttggcccctggggtcctgcctcatgaatgaggccttccccagggctggcctgactgtgctgggggctgggttaacgttttctcagggaaccacaatgcacgaaagaggaactggggttgctaaccaggatgctgggaacaaaggcctcttgaagcccagccacagcccagctgagcatgaggcccagcccatagacggcacaggccacctggcccattccctgggcattccctgctttgcattgctgcttctcttcaccccatggaggctatgtcaccctaactatcctggaatgtgttgagagggattctgaatgatcaatatagcttggtgagacagtgccgagatagatagccatgtctgccttgggcacgggagagggaagtggcagcatgcatgctgtttcttggccttttctgttagaatacttggtgctttccaacacactttcacatgtgttgtaacttgtttgatccacccccttccctgaaaatcctgggaggttttattgctgccatttaacacagagggcaatagaggttctgaaaggtctgtgtcttgtcaaaacaagtaaacggtggaactacgactaaa

SEQ ID NO: 50 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 51 - RNA, 2666 nt, Homo sapiens
  source 1..2666: mol_type = mRNA
agttctcttcaagtcatgtaatcgacttttttgaattagttttcagtttcattttgttttccctaattcaagttgggaacacttcattttccccaattcaagttgggaacacttccttggtatttccttgctacatggactttagcaaatgctactttactctccttccagctactcaggaggctgaggcaggagaatcgcttgaacccgggaggcggaggttacagtgagccttttcctagttttactgttggaagcctaactcacaggagagattatgcaatacagtcctgaagtcaagggaggagagcatgtaggagaatactaaccctgcacagattgtgatggtgatgtggaatatactaaagcctagaacgcacctcctctgcatgactaatatgttctgcacaagacatgaaggcacagacagcactgtctttcttcctcattctcataacatctctgagtggatctcaaggcatattccctttggctttcttcatttatgttcctatgaatgaacaaatcgtcattggaagacttgatgaagatataattctcccttcttcatttgagaggggatccgaagtcgtaatacactggaagtatcaagatagctataaggttcacagttactacaaaggcagtgaccatttggaaagccaagatcccagatatgcaaacaggacatcccttttctataatgagattcaaaatgggaatgcgtcgctatttttcagaagagtaagccttctggacgaaggaatttacacctgctatgtaggaacagcaattcaagtgattacaaacaaagtggtgctaaaggtgggagtttttctcacacccgtgatgaagtatgaaaagaggaacacaaacagcttcttaatatgcagcgtgttaagtgtttatcctcgtccaattatcacgtggaaaatggacaacacacctatctctgaaaacaacatggaagaaacagggtctttggattctttttctattaacagcccactgaatattacaggatcaaattcatcttatgaatgtacaattgaaaattcactgctgaagcaaacatggacagggcgctggacgatgaaagatggccttcataaaatgcaaagtgaacacgtttcactctcatgtcaacctgtaaatgattatttttcaccaaaccaagacttcaaagttacttggtccagaatgaaaagtgggactttctctgtcctggcttactatctgagctcctcacaaaatacaattatcaatgaatcccgattctcatggaacaaagagctgataaaccagagtgacttctctatgaatttgatggatcttaatctttcagacagtggggaatatttatgcaatatttcttcggatgaatatactttacttaccatccacacagtgcatgtagaaccgagccaagaaacagcttcccataacaaaggcttatggattttggtgccctctgcgattttggcagcttttctgctgatttggagcgtaaaatgttgcagagcccagctagaagccaggaggagcagacaccctgctgatggagcccaacaagaaagatgttgtgtccctcctggtgagcgctgtcccagtgcacccgataatggcgaagaaaatgtgcctctttcaggaaaagtataggaaatgagagaagactgtgacaactcatgacctgcatccttaatatccagtgacttcatctcccctttcttcaccacaattccaggcaatggcctgtcggagcagacaattctaccactgcaaagagttgtaaccattttctggtatcacatttatttttcaagacatacttttcaagacatcattcactgacccactacctgcattgagtataaatgcctggatgttaaggattccaatttaactttgaaaagaactgtctcattcatttacatttctgttacagtcagcccaggaggttacagtgagctctccactaagaatctggaagaaatgcatcactaggggttgattcccaatctgatcaactgataatgggtgagagagcaggtaagagccaaagtcaccttagtggaaaggttaaaaaccagagcctggaaaccaagatgattgatttgacaaggtattttagtctagttttatatgaacggttgtatcagggtaaccaactcgatttgggatgaatcttagggcaccaaagactaagacagtatctttaagattgctagggaaaagggccctatgtgtcaggcctctgagcccaagccaagcatcgcatcccctgtgatttgcacgtatacatccagatggcctaaagtaactgaagatccacaaaagaagtaaaaatagccttaactgatgacattccaccattgtgatttgttcctgccccaccctaactgatcaatgtactttgtaatctcccccacccttaagaaggtactttgtaatcttccccacccttaagaaggttctttgtaattctccccacccttgagaatgtactttgtgagatccaccctgcccacaaaacattgctcttaacttcaccgcctaacccaaaacctataagaactaatgataatccatcacccttcgctgactctcttttcggactcagcccacctgcacccaggtgaaataaacagctttattgctcacacaaa

SEQ ID NO: 52 - protein, 414 aa, Homo sapiens
MKAQTALSFFLILITSLSGSQGIFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 53 - RNA, 1818 nt, Homo sapiens
  source 1..1818: mol_type = mRNA
actcattgcaccttcctgccaccccaggcagtgtctgggccctcagctccccctccctccacctaccccctcacacccaccactacgaccccacgggatacccagcccagacggaggaaacaccgagcctagagacatgagagttggaggagcattccaccttctactcgtgtgcctgagcccagcactgctgtctgctgtgcggatcaacggggatggacaggaggtcctgtacctggcagaaggtgataatgtgaggctgggctgcccctacgtcctggaccctgaggactatggtcccaatgggctggacatcgagtggatgcaggtcaactcagaccccgcccaccaccgagagaacgtgttccttagttaccaggacaagaggatcaaccatggcagccttccccatctgcagcagagggtccgctttgcagcctcagacccaagccagtacgatgcctccatcaacctcatgaacctgcaggtatctgatacagccacttatgagtgccgggtgaagaagaccaccatggccacccggaaggtcattgtcactgtccaagcacgacctgcagtgcccatgtgctggacagagggccacatgacatatggcaacgatgtggtgctgaagtgctatgccagtgggggctcccagcccctctcctacaagtgggccaagatcagtgggcaccattacccctatcgagctgggtcttacacctcccagcacagctaccactcagagctgtcctaccaggagtccttccacagctccataaaccaaggcctgaacaatggggacctggtgttgaaggatatctccagagcagatgatgggctgtatcagtgcacagtggccaacaacgtgggctacagtgtttgtgtggtggaggtgaaggtctcagactcccggcgtataggcgtgatcatcggcatcgtcctgggctctctgctcgcgctgggctgcctggccgtaggcatctgggggctcgtctgctgctgctgcgggggctccggggctggcggcgcccgcggtgccttcggctacggcaacggcggcggggtcggcggaggggcctgcggcgacttggctagtgagatcagagaggacgccgtggcgcccgggtgcaaggccagcgggcgcggcagccgcgtcacccacctcctggggtacccgacgcagaacgtcagccgctccctgcgccgcaagtacgcgcctcccccctgcggcggccccgaggacgtggccctggcgccctgcaccgccgccgccgcctgcgaagcgggcccctccccggtctacgtcaaggtcaagagcgcggagccggctgactgcgccgaggggccggtgcagtgcaagaacggcctcttggtgtgagcgcgcgcgccgggccgggctgcgccccagccaggaggagggcgcggggctctctgtctgcagctggggacacgtcggggctggggacgacctcgctcgccccaggctgccaggcggctgggggtgaaggcatttccctaaggaaatgcgtagggaggcagagcctcctccccaaaagtgggaaggggcgggcgagggcggaggaaggcgatcctgagccttctccgcacccccgggaccgaaggcttgggggagagggagggaggaggaggctgagtgtcctagagcggctgaggccggaggcctggtgtccccagcctaagcagagggccccgggggccgggtgggtgggggtctgtctggacgaattgttctgtgtgtgaggtctgagctctgaggcagcagtgttagcacaataaagaaacattgagacgtga

SEQ ID NO: 54 - protein, 414 aa, Homo sapiens
MRVGGAFHLLLVCLSPALLSAVRINGDGQEVLYLAEGDNVRLGCPYVLDPEDYGPNGLDIEWMQVNSDPAHHRENVFLSYQDKRINHGSLPHLQQRVRFAASDPSQYDASINLMNLQVSDTATYECRVKKTTMATRKVIVTVQARPAVPMCWTEGHMTYGNDVVLKCYASGGSQPLSYKWAKISGHHYPYRAGSYTSQHSYHSELSYQESFHSSINQGLNNGDLVLKDISRADDGLYQCTVANNVGYSVCVVEVKVSDSRRIGVIIGIVLGSLLALGCLAVGIWGLVCCCCGGSGAGGARGAFGYGNGGGVGGGACGDLASEIREDAVAPGCKASGRGSRVTHLLGYPTQNVSRSLRRKYAPPPCGGPEDVALAPCTAAAACEAGPSPVYVKVKSAEPADCAEGPVQCKNGLLV

SEQ ID NO: 55 - RNA, 3523 nt, Homo sapiens
  source 1..3523: mol_type = mRNA
agtcctggggcagggctgggtggcacggctggcgagcccggaacgcctctggtcacagctcagcgtccgcggagccgggcggcgctgcagctgcacttggctcgtctgtgggtctgacagtcccagctctgcgcggggaacagcggcccggcgctgggtgtgggaggaccaggctgccccaagagcgcggagactcacgcccgctcctctcctgttgcgaccgggagccgggtaggaggcaggcgcgctccctgcggccccgggatgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtataggacatgaggaaatgttgtgttcagaaatgaataaatggaatgccctcatacaagggggagggtggggtggggagtgctgggaaagaaacacttccttataattatattagtaaaatgcacaaagaagaaggcagtgctgttacttggccactaagatgtgtaaaatggactgaaatgctccatcatgaagacttgcttccccaccaaagatgtcctgggattctgctggatctcaaagatgtgccaagccaaggaaaaagatacaagagcagaatagtacttaaaatccaaactgccgcccagatgggcttgttcttcatgcctaacttaataatttttaagagattaaagtgccagatggagtttaaatattgaaattattttaaaaggtaggtgtctttaagaaaataacaagcaaccctgtgatatgttccgtctctcccaattccctcgttatatagagggcttaatggtataaatggttaatattggtcccaacagggctgactcttctatcatataatcaaaactttttacatgagcaaaattcagtaagaaatgggggaagacaaaggaaacgtctttgagaagccccttcatatttatttatttatctcttcctgaaccatgaatttcatatgtggaatattgctatattgacagattcttgcctgtctgtgttattctaggatctgttacaggtccatggcaattactgtttattttttcctggaaaaatatttttttataaaaggcttttttttttttttaaatacatgagaggcattgggctaagaaagaaaagactgttgtataataccttgttcaatggttgtatttagtgagctcatagaggtccatcatatcatgaccgagctaggttgtgtgggcaggaaggtagggctaaggggttgtagccttgctgggcagcctctcagagcaaggttgttcagatctcccttgctattacagtaggttactattaatgagggcagcacctgatgccttttgtactgaggtatgtaactttctccttatttgacaagtagaagttaacttacttgtcagggagggcagacgtttttttgttctgtttcgtttttcaaaataatgctttttgcaaaagaggtaagactgagactaaaggtgttatcttctggtgtgctcctggaagtgtctaccctacatttgtgtcagctcagggttgcagtgttgcccagatgcattttacatcactgtaaagagattacttttgtggttactacctggcttggctggccttgcggttcaccagattaatttacaaactcccccactttattttgtgctatgtagatctggccatacttgcattagtgactgtcttgccttaaccacacttaagcaacccacaaatttcttctcagatttgtttcctagattacttatgatactcatcccatgtctcaataagagtgtcttttctttctggatgtgttctcttactccctcttaccaccatactttttgctctcttctcctgcaagcgtagtcttcacagggagtggcttcctgacatttttttcagttatgtgaatgaatggaaaccaacagctgctgcaaacactgtttttccaagaaggctacactcagaacctaaccattgccaaccatttcagtattgataaaaagctgaatttactttagcattacttatttttttttccatttgatggttcttactttgtaaaaatttaaataaatgaatgtctatactttttataaagaaaagtgaaaataccatgacactgaaaagatgatgctatcagatgctgtttagaaagcatttatcttgcatttctttattctttctaattatctaaaattcaataaaattttattcatataaaataagttgtcattaattatcaatactaacgagtatgtcattttaaaacttagtattctctttaatgttacaaga

SEQ ID NO: 56 - protein, 431 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 57 - RNA, 1807 nt, Homo sapiens
  source 1..1807: mol_type = mRNA
acagacgctggcggccaccagaagtttgagcctctttggtagcaggaggctggaagaaaggacagaagtagctctggctgtgatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcctgtctttgccatcatcctcatcatctccttgtgctgtatggtggtttttaccatggcctatatcatgctctgtcggaagacatcccaacaagagcatgtctacgaagcagccagggcacatgccagagaggccaacgactctggagaaaccatgagggtggccatcttcgcaagtggctgctccagtgatgagccaacttcccagaatctgggcaacaactactctgatgagccctgcataggacaggagtaccagatcatcgcccagatcaatggcaactacgcccgcctgctggacacagttcctctggattatgagtttctggccactgagggcaaaagtgtctgttaaaaatgccccattaggccaggatctgctgacataattgcctagtcagtccttgccttctgcatggccttcttccctgctacctctcttcctggatagcccaaagtgtccgcctaccaacactggagccgctgggagtcactggctttgccctggaatttgccagatgcatctcaagtaagccagctgctggatttggctctgggcccttctagtatctctgccgggggcttctggtactcctctctaaataccagagggaagatgcccatagcactaggacttggtcatcatgcctacagacactattcaactttggcatcttgccaccagaagacccgagggaggctcagctctgccagctcagaggaccagctatatccaggatcatttctctttcttcagggccagacagcttttaattgaaattgttatttcacaggccagggttcagttctgctcctccactataagtctaatgttctgactctctcctggtgctcaataaatatctaatcataacagcaa

SEQ ID NO: 58 - protein, 399 aa, Homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 59 - RNA, 2237 nt, Homo sapiens
  source 1..2237: mol_type = mRNA
atttggagagttaaaactgtgcctaacagaggtgtcctctgacttttcttctgcaagctccatgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatagatccaaccaccttatttttgagcttggtgttttgtctttttcagaaactatgagctgtgtcacctgactggttttggaggttctgtccactgctatggagcagagttttcccattttcagaagataatgactcacatgggaattgaactgggacctgcactgaacttaaacaggcatgtcattgcctctgtatttaagccaacagagttacccaacccagagactgttaatcatggatgttagagctcaaacgggcttttatatacactaggaattcttgacgtggggtctctggagctccaggaaattcgggcacatcatatgtccatgaaacttcagataaactagggaaaactgggtgctgaggtgaaagcataacttttttggcacagaaagtctaaaggggccactgattttcaaagagatctgtgatccctttttgttttttgtttttgagatggagtcttgctctgttgcccaggctggagtgcaatggcacaatctcggctcactgcaagctccgcctcctgggttcaagcgattctcctgcctcagcctcctgagtggctgggattacaggcatgcaccaccatgcccagctaatttgttgtatttttagtagagacagggtttcaccatgttggccagtgtggtctcaaactcctgacctcatgatttgcctgcctcggcctcccaaagcactgggattacaggcgtgagccaccacatccagccagtgatccttaaaagattaagagatgactggaccaggtctaccttgatcttgaagattcccttggaatgttgagatttaggcttatttgagcactgcctgcccaactgtcagtgccagtgcatagcccttcttttgtctcccttatgaagactgccctgcagggctgagatgtggcaggagctcccagggaaaaacgaagtgcatttgattggtgtgtattggccaagttttgcttgttgtgtgcttgaaagaaaatatctctgaccaacttctgtattcgtggaccaaactgaagctatatttttcacagaagaagaagcagtgacggggacacaaattctgttgcctggtggaaagaaggcaaaggccttcagcaatctatattaccagcgctggatcctttgacagagagtggtccctaaacttaaatttcaagacggtataggcttgatctgtcttgcttattgttgccccctgcgcctagcacaattctgacacacaattggaacttactaaaaatttttttttactgtt

SEQ ID NO: 60 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 61 - RNA, 1330 nt, Homo sapiens
  source 1..1330: mol_type = mRNA
agactcctgggtccggtcaaccgtcaaaatgtccaaagaacctctcattctctggctgatgattgagttttggtggctttacctgacaccagtcacttcagagactgttgtgacggaggttttgggtcaccgggtgactttgccctgtctgtactcatcctggtctcacaacagcaacagcatgtgctgggggaaagaccagtgcccctactccggttgcaaggaggcgctcatccgcactgatggaatgagggtgacctcaagaaagtcagcaaaatatagacttcaggggactatcccgagaggtgatgtctccttgaccatcttaaaccccagtgaaagtgacagcggtgtgtactgctgccgcatagaagtgcctggctggttcaacgatgtaaagataaacgtgcgcctgaatctacagagagcctcaacaaccacgcacagaacagcaaccaccaccacacgcagaacaacaacaacaagccccaccaccacccgacaaatgacaacaaccccagctgcacttccaacaacagtcgtgaccacacccgatctcacaaccggaacaccactccagatgacaaccattgccgtcttcacaacagcaaacacgtgcctttcactaaccccaagcacccttccggaggaagccacaggtcttctgactcccgagccttctaaggaagggcccatcctcactgcagaatcagaaactgtcctccccagtgattcctggagtagtgttgagtctacttctgctgacactgtcctgctgacatccaaagagtccaaagtttgggatctcccatcaacatcccacgtgtcaatgtggaaaacgagtgattctgtgtcttctcctcagcctggagcatctgatacagcagttcctgagcagaacaaaacaacaaaaacaggacagatggatggaatacccatgtcaatgaagaatgaaatgcccatctcccaactactgatgatcatcgccccctccttgggatttgtgctcttcgcattgtttgtggcgtttctcctgagagggaaactcatggaaacctattgttcgcagaaacacacaaggctagactacattggagatagtaaaaatgtcctcaatgacgtgcagcatggaagggaagacgaagacggcctttttaccctctaacaacgcagtagcatgttagattgaggatgggggcatgacactccagtgtcaaaataagtcttagtagatttccttgtttcataaaaaagactcacttattccatggatgtcattgatccaggcttgctttagtttcatgaatgaagggtactttagagaccacaa

SEQ ID NO: 62 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 63 - RNA, 3491 nt, Homo sapiens
  source 1..3491: mol_type = mRNA
agcacagagagtggaaaacagcagaggtgacagagcagccgtgctcgaagcgttcctggagcccaagctctcctccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcga

SEQ ID NO: 64 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 65 - RNA, 3388 nt, Homo sapiens
  source 1..3388: mol_type = mRNA
attcctcacgatgacccgacagtctctgctttctttttcctttcttccagaaggagatttaaccatagtagaaagaatggagaactattaactgcctttcttctgtgggctgtgattttcagaggggaatgctaagaggtgattttcaatgttgggactcaaaggtgaagacactgaaggacagaatttttggcagaggaaagatcttcttcggtcaccatacttgagttagctctagggaagtggaggtttccatttggaattctatagcttcttccaggtcatagtgtctgccccccaccttccagtatctcctgatatgcagcatgaatgaaaatggcaagtttcctggccttccttctgctcaactttcgtgtctgcctccttttgcttcagctgctcatgcctcactcagctcagttttctgtgcttggaccctctgggcccatcctggccatggtgggtgaagacgctgatctgccctgtcacctgttcccgaccatgagtgcagagaccatggagctgaagtgggtgagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctgatcttcacgttgatgtgaagggttacaaggatggagggatccatctggagtgcaggtccactggctggtacccccaaccccaaatacagtggagcaacaacaagggagagaacatcccgactgtggaagcacctgtggttgcagacggagtgggcctgtatgcagtagcagcatctgtgatcatgagaggcagctctggggagggtgtatcctgtaccatcagaagttccctcctcggcctggaaaagacagccagcatttccatcgcagaccccttcttcaggagcgcccagaggtggatcgccgccctggcagggaccctgcctgtcttgctgctgcttcttgggggagccggttacttcctgtggcaacagcaggaggaaaaaaagactcagttcagaaagaaaaagagagagcaagagttgagagaaatggcatggagcacaatgaagcaagaacaaagcacaagagtgaagctcctggaggaactcagatggagaagtatccagtatgcatctcggggagagagacattcagcctataatgaatggaaaaaggccctcttcaagcctgcggatgtgattctggatccaaaaacagcaaaccccatcctccttgtttctgaggaccagaggagtgtgcagcgtgccaaggagccccaggatctgccagacaaccctgagagatttaattggcattattgtgttctcggctgtgagagcttcatatcagggagacattactgggaggtggaggtaggggacaggaaagagtggcatataggggtgtgcagtaagaatgtgcagagaaaaggctgggtcaaaatgacacctgagaatggattctggactatggggctgactgatgggaataagtatcggactctaactgagcccagaaccaacctgaaacttcctaagccccctaagaaagtgggggtcttcctggactatgagactggagatatctcattctacaatgctgtggatggatcgcatattcatactttcctggacgtctccttctctgaggctctatatcctgttttcagaattttgaccttggagcccacggccctgactatttgtccagcgtgaaaagaagaagagagttcctccaattctgaccgagtgctgatcattccctagagacaccagtaaccccgggcttagctaacgaaagtggggagcctcaggctgaagtaacttttctctgcttctccctgcccagctcagagctgagggcctccccctccacagcaaccaatcacaaccataaagctacaagcacgcactgaagcactttactgatactcattcaattattcatatgacagttgtttgagtttggtaccatcttattttccccttatacagataaggaaactggggtgcagaaaagtgaattgactacaaagtagacatgactagttaacaacacagctgggatctaaacagcaataactaacattaatggagaacttaaaatgctctgagtgctgtgttatgagctttggtggatgtcactcctttaatcctcgcaacaccctgtcgggtagtctcatttagcaagtatggaagttgaggcagggcaacattaagcaacttacataactcatgcagtaatttctgcagttgggagatgttcagcttcagtccccggccctatggccgttcttttccaccctgtttcttcccccataggaagaacccacctgtagccctgaggttcttttcccaggatggctccaggataaggatcactgtaggtggttgtggagttgacacccctgttgactccttcccagctgattgtcagagccttagacccagcacgccttggattagctctgcagagtgtcttggttgagagaataacctcaccgtacccacatgacacgtgatttggaaagagactagaggccacacttgataaatcatggggaacagatgtgttccacccaacaaatgtgataagtgatcatgcagccagagccagccttccttcaatcaaggtttccaggcagagcaaataccctagagattctctgtgatataggaaatttggatgaagggagctagaagaaatacagggatttttttttttttttaagatggagtcttactctgttgctaggctggagtgcagtggtgcgatctcagctccctgcaacctccacctcctgggttcaaacaattctcctgcctcagcctcccgagtactgggaatataggtgcacgccaccacacccaacaaatttttgtacttttagtacagatgagggttcactatgttggccaggatggtctcgatctcttgacctcatgatccacccacctcggtctcccaaagtgctgggattacaggcttgagccaccgggtgaccggcttacagggatatttttaatcccgttatggactctgtctccaggagaggggtctatccacccctgctcattggtggatgttaaaccaatattcctttcaactgctgcctgctagggaaaaactactcctcattatcatcattattattgctctccactgtatcccctctacctggcatgtgcttgtcaagttctagttgttcaataaatttgttaataatgctga

SEQ ID NO: 66 - protein, 513 aa, Homo sapiens
MKMASFLAFLLLNFRVCLLLLQLLMPHSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTLPVLLLLLGGAGYFLWQQQEEKKTQFRKKKREQELREMAWSTMKQEQSTRVKLLEELRWRSIQYASRGERHSAYNEWKKALFKPADVILDPKTANPILLVSEDQRSVQRAKEPQDLPDNPERFNWHYCVLGCESFISGRHYWEVEVGDRKEWHIGVCSKNVQRKGWVKMTPENGFWTMGLTDGNKYRTLTEPRTNLKLPKPPKKVGVFLDYETGDISFYNAVDGSHIHTFLDVSFSEALYPVFRILTLEPTALTICPA

SEQ ID NO: 67 - RNA, 3776 nt, Homo sapiens
  source 1..3776: mol_type = mRNA
gactcttactgtttctcatggtgagaagacaatatttgctttctctttttcctttcttccggatgagaggctaagccataatagaaagaatggagaattattgattgaccgtctttattctgtgggctctgattctccaatgggaataccaagggatggttttccatactggaacccaaaggtaaagacactcaaggacagacatttttggcagagcatagatgaaaatggcaagttccctggctttccttctgctcaactttcatgtctccctcctcttggtccagctgctcactccttgctcagctcagttttctgtgcttggaccctctgggcccatcctggccatggtgggtgaagacgctgatctgccctgtcacctgttcccgaccatgagtgcagagaccatggagctgaagtgggtaagttccagcctaaggcaggtggtgaacgtgtatgcagatggaaaggaagtggaagacaggcagagtgcaccgtatcgagggagaacttcgattctgcgggatggcatcactgcagggaaggctgctctccgaatacacaacgtcacagcctctgacagtggaaagtacttgtgttatttccaagatggtgacttctatgaaaaagccctggtggagctgaaggttgcagcactgggttctaatcttcacgtcgaagtgaagggttatgaggatggagggatccatctggagtgcaggtccaccggctggtacccccaaccccaaatacagtggagcaacgccaagggagagaacatcccagctgtggaagcacctgtggttgcagatggagtgggcctatatgaagtagcagcatctgtgatcatgagaggcggctccggggagggtgtatcctgcatcatcagaaattccctcctcggcctggaaaagacagccagcatttccatcgcagaccccttcttcaggagcgcccagccctggatcgcagccctggcagggaccctgcctatcttgctgctgcttctcgccggagccagttacttcttgtggagacaacagaaggaaataactgctctgtccagtgagatagaaagtgagcaagagatgaaagaaatgggatatgctgcaacagagcgggaaataagcctaagagagagcctccaggaggaactcaagaggaaaaaaatccagtacttgactcgtggagaggagtcttcgtccgataccaataagtcagcctgatgctctaatggaaaaatggccctcttcaagcctggtgaggaaatgcttcagatgaggctccaccttgttaaataaattggatgtatggaaaaatagactgcagaaaaggggaactcatttagctcacgagtggtcgagtgaagattgaaaattaacctctgagggccagcacagcagctcatgcctgtaatcctagcactttggaaggctgaggagggcggatcacaaggtcaggagatcaagaccatcctggctaacacggtgaaaccccgtctctactaaaaatacaaaaaataaaaaattagccgggcatggtgacgggcacctgtagtcccagctactcgggaggctgaggcaggagaatggcatgaacccggaaggcagagcttgcagtgagccgagatcacgccactgcactccagcctgggagacagagcgagactctgtctcaagaaaaaaaaaaaaaaaaaaaaagaaaagaaaattaacctctgagtataaagcatcagtgggcagaatcaatgtggggagggaaacaacaaaaatgtagaaagaggatccttgttgcttcttggggccgcatcagggtattgggttaggcagatactgaccttactttcatttcccctctggtcactagacccctggggctttcaccaatgacattgatgagagaatcacattcagggcaggctagggacacggggttctggaaggacctcctcagcatggcccaagccttgcatgctgtggctcttaaatccaggaaaaatggctgaccccatggacacctcctcaaactctctgcagcagatgtaattctgtatccagacatggcaaatgccatcctccttgtttctgaggaccagaggagtgtacagcgtgctgaggagccccatgacctaccagacaaccctgagagatttgaatggcgttactgtgtgcttggctgtgaaagcttcatgtcagagagacactactgggaggtggaagtgggggacagaaaagagtggcatattggggtatgtagtaagaacgtggagaggaaaaaagtttgggtcaaaatgacaccggagaacggatactggactatgggcctgactgatgggaataagtatcgggctctcactgagcccagaaccaacctgaaacttcctgagcctcctaggaaagtgggggtcatcctggactatgagactggacatatctcgttctacaatgccacggatggatctcatatctacacatttctgcacgcctcttcctctgagcctctgtatcctgtattcagaattttgaccttggagcccactgccctgaccgtttgcccaataccaaaagtagagagttcccccgatcccgacctagtgcctgatcattccctggagataccactgaccccaggcttagctaatgaaagtggggagcctcaggctgaagtaacatctctgcttctccctgcccagcctggagctaagggtctcaccctccacaacagccagtcagaaccataaagctacaggcacacactgaagcactttactgatattcattcaattattccataggacagttgtttgagtttggtgccaccttattggcccctttatacagataaggaaactggggtgtagaaaagtgtattgactttacaaagcagacaggaatagtgaacaacagagctgggatctgaacaacaatgactaacattaatggagaatttaaaacgttctgagtgctgtgttatgagctttggtgggtgtcactcctttaatcctcacaacaccctgtcaggtagtctcatttggcaagtatggaagcagaggcagggcaacattaagtagcttacataactcacacggtaatttgtgcagttgggagatgttcagcttcagtccctggccaattgcccgttcttttccagcctgatttttcctgcatgggaagagcccacatgtagccctgaggttcccttcccaggacagctccaggatcgagatcactgtgagtggttgtggagttaagacccctatggactccttcccagctgattatcagagccttagacccagcactccttggattggctctgcagagtgtcttggttgagagaataacgttgcagttcccacagggcatgtgactttgaaagagactagaggccacactcagttaataatggggcacagatgtgttcccacccaacaaatgtgataagtgatcgtgcagccagagccagccttccttcagtcaaggtttccaggcagagcaaataccctagagattctctgtaatattggtaatttggatgaaggaagctagaagaattacagggatgtttttaatcccactatggactcagtctcctggaaaaggatctgtccactcctggtcattggtggatgttaaacccatattcctttcaactgctgcctgctagggaaaactgctcctcattatcatcactattattgctcaccactgtatcccctctactgggcaagtgcttgtcaagttctagttgttcaataaatttgttaataatgctga

SEQ ID NO: 68 - protein, 334 aa, Homo sapiens
MKMASSLAFLLLNFHVSLLLVQLLTPCSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSNLHVEVKGYEDGGIHLECRSTGWYPQPQIQWSNAKGENIPAVEAPVVADGVGLYEVAASVIMRGGSGEGVSCIIRNSLLGLEKTASISIADPFFRSAQPWIAALAGTLPILLLLLAGASYFLWRQQKEITALSSEIESEQEMKEMGYAATEREISLRESLQEELKRKKIQYLTRGEESSSDTNKSA

SEQ ID NO: 69 - RNA, 2890 nt, Homo sapiens
  source 1..2890: mol_type = mRNA
agatttcgtttcctgcatctccaaacatggcgacctaggagaaggggaagaacaattttttctcctcttttgggaaggtttgtgtctagtagtgcctgtgcccctgggcagattggagagaagagggacgactggagaatcgtcgagaaccagcggagaaaagaaaaagcaacgtttaattctagaaggcctcctgtccctgcctgctctgggtgctcatggaatcagctgctgccctgcacttctcccggccagcctccctcctcctcctcctcctcagcctgtgtgcactggtctcagcccagtttattgtcgtggggcccactgatcccatcttggccacggttggagaaaacactacgttacgctgccatctgtcacccgagaaaaatgctgaggacatggaggtgcggtggttccggtctcagttctcccccgcagtgtttgtgtataaaggtggcagagagagaacagaggagcagatggaggagtaccgaggaagaaccacctttgtgagcaaagacatcagcaggggcagcgtggccctggtcatacacaacatcacagcccaggaaaacggcacctaccgctgttacttccaagaaggcaggtcctacgatgaggccatcctgcacctcgtagtggcaggactaggctctaagcccctcatttcaatgaggggccatgaagacgggggcatccggctggagtgcatatctagagggtggtacccaaagcccctcacagtgtggagggacccctacggtggggttgcgcctgccctgaaagaggtctccatgcctgatgcagacggcctcttcatggtcaccacggctgtgatcatcagagacaagtctgtgaggaacatgtcctgctctatcaacaacaccctgctcggccagaagaaagaaagtgtcatttttattccagaatcctttatgcccagtgtgtctccctgtgcagtggccctgcctatcattgtggttattctgatgatacccattgccgtatgcatctattggatcaacaaactccaaaaggaaaaaaagattctgtcaggggaaaaggagtttgaacgggaaacaagagaaattgctctaaaggaactggagaaagaacgtgtgcaaaaagaggaagaacttcaagtaaaagagaaacttcaagaagaattgcgatggagaagaacattcttacatgctgttgatgtggtcctggatccagacaccgctcatcccgatctcttcctgtcagaggaccggagaagtgtgagaaggtgccccttcaggcacctaggggagagcgtgcctgacaacccagagagattcgacagtcagccttgtgtcctaggccgggagagcttcgcttcagggaaacattactgggaggtggaggtggaaaacgtgattgagtggactgtgggggtctgtagagacagtgttgagaggaaaggggaggtcctgctgattcctcagaatggcttctggaccttggagatgcataaagggcaataccgggccgtgtcctcccctgataggattctccctttgaaggagtccctttgccgggtgggcgtcttcctggactatgaagctggagatgtctccttctacaacatgagggacagatcgcacatctacacatgtccccgttcagccttttccgtgcctgtgaggcccttcttcaggttggggtgtgaggacagccccatcttcatctgccctgcactcacaggagccaatggggtcacggtgcctgaagagggcctgacacttcacagagtggggacccaccagagcctatagaatcaattccttggtctcacagccatgtagacaagccctggtcatctcagcagccaccgcacaacacccctggtggaagacacgccctcctcccctctggtcacacaagagaacatcttccagctgcctctttcacacccactacagacctcagccccagttttctcctcctcactaggctgtgtttttagtagttcctttgcttgtaactatgggatgggatccaggcatagggaactagttgttacacagctcccagccaagaagaaagtgtgagaagttgatgggcagcaaacctgctgtttaacatcagggtgaccacattaagcccagtattccagttggcaccagaagatatggacttggaatgaggcctacagggttcaccaggatgtaagaggagagaggaatccacaggaccaccagagaggagagggaaccagatatgcagatcagagatagaggaagtggaaccagagagctgggagggaccaaggttgtaagggtggctaagtcccaccataacagctaaggggacctgggagatgatggctcatttccacccagccccaggatttccagagcgcacatccacaggcctggacctgggatgaagatgaatgaagaacatggatgcacgtggatgtagtttggctcaggtgtccctgcagttggcaaggagtcagtactcagtccctgagtgtggctgaaatttgaggtcctggctgagccaaggagtaatggaccagatctacctcagtattcaagttcagtggggacaccagtggcttcaaacttcctggtttcatgatatcttgagacgccttacaaatgatggaggattccaaagagtttttgtttatttgggttaatatttgttggtatttatggcatttgagattgaaactaagaaatgttttaatttattacctttacaacatttatttacattacatacatacatttacaacatttattaatttatattaaaatagcatgaataagccaattataggttaatataagtagaatgtttgtgaaaaataagtatggtatccaaagcaaaataaattttattgtgaagtgtg

SEQ ID NO: 70 - protein, 527 aa, Homo sapiens
MESAAALHFSRPASLLLLLLSLCALVSAQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAVALPIIVVILMIPIAVCIYWINKLQKEKKILSGEKEFERETREIALKELEKERVQKEEELQVKEKLQEELRWRRTFLHAVDVVLDPDTAHPDLFLSEDRRSVRRCPFRHLGESVPDNPERFDSQPCVLGRESFASGKHYWEVEVENVIEWTVGVCRDSVERKGEVLLIPQNGFWTLEMHKGQYRAVSSPDRILPLKESLCRVGVFLDYEAGDVSFYNMRDRSHIYTCPRSAFSVPVRPFFRLGCEDSPIFICPALTGANGVTVPEEGLTLHRVGTHQSL

SEQ ID NO: 71 - RNA, 2017 nt, Homo sapiens
  source 1..2017: mol_type = mRNA
agaacagcgcagtttgccctccgctcacgcagagcctctccgtggcttccgcaccttgagcattaggccagttctcctcttctctctaatccatccgtcacctctcctgtcatccgtttccatgccgtgaggtccattcacagaacacatccatggctctcatgctcagtttggttctgagtctcctcaagctgggatcagggcagtggcaggtgtttgggccagacaagcctgtccaggccttggtgggggaggacgcagcattctcctgtttcctgtctcctaagaccaatgcagaggccatggaagtgcggttcttcaggggccagttctctagcgtggtccacctctacagggacgggaaggaccagccatttatgcagatgccacagtatcaaggcaggacaaaactggtgaaggattctattgcggaggggcgcatctctctgaggctggaaaacattactgtgttggatgctggcctctatgggtgcaggattagttcccagtcttactaccagaaggccatctgggagctacaggtgtcagcactgggctcagttcctctcatttccatcacgggatatgttgatagagacatccagctactctgtcagtcctcgggctggttcccccggcccacagcgaagtggaaaggtccacaaggacaggatttgtccacagactccaggacaaacagagacatgcatggcctgtttgatgtggagatctctctgaccgtccaagagaacgccgggagcatatcctgttccatgcggcatgctcatctgagccgagaggtggaatccagggtacagataggagatacctttttcgagcctatatcgtggcacctggctaccaaagtactgggaatactctgctgtggcctattttttggcattgttggactgaagattttcttctccaaattccagtggaaaatccaggcggaactggactggagaagaaagcacggacaggcagaattgagagacgcccggaaacacgcagtggaggtgactctggatccagagacggctcacccgaagctctgcgtttctgatctgaaaactgtaacccatagaaaagctccccaggaggtgcctcactctgagaagagatttacaaggaagagtgtggtggcttctcagagtttccaagcagggaaacattactgggaggtggacggaggacacaataaaaggtggcgcgtgggagtgtgccgggatgatgtggacaggaggaaggagtacgtgactttgtctcccgatcatgggtactgggtcctcagactgaatggagaacatttgtatttcacattaaatccccgttttatcagcgtcttccccaggaccccacctacaaaaataggggtcttcctggactatgagtgtgggaccatctccttcttcaacataaatgaccagtcccttatttataccctgacatgtcggtttgaaggcttattgaggccctacattgagtatccgtcctataatgagcaaaatggaactcccatagtcatctgcccagtcacccaggaatcagagaaagaggcctcttggcaaagggcctctgcaatcccagagacaagcaacagtgagtcctcctcacaggcaaccacgcccttcctccccaggggtgaaatgtaggatgaatcacatcccacattcttctttagggatattaaggtctctctcccagatccaaagtcccgcagcagccggccaaggtggcttccagatgaagggggactggcctgtccacatgggagtcaggtgtcatggctgccctgagctgggagggaagaaggctgacattacatttagtttgctctcactccatctggctaagtgatcttgaaataccacctctcaggtgaagaaccgtcaggaattcccatctcacaggctgtggtgtagattaagtagacaaggaatgtgaataatgcttagatcttattgatgacagagtgtatcctaatggtttgttcattatattacactttcagtaa

SEQ ID NO: 72 - protein, 500 aa, Homo sapiens
MALMLSLVLSLLKLGSGQWQVFGPDKPVQALVGEDAAFSCFLSPKTNAEAMEVRFFRGQFSSVVHLYRDGKDQPFMQMPQYQGRTKLVKDSIAEGRISLRLENITVLDAGLYGCRISSQSYYQKAIWELQVSALGSVPLISITGYVDRDIQLLCQSSGWFPRPTAKWKGPQGQDLSTDSRTNRDMHGLFDVEISLTVQENAGSISCSMRHAHLSREVESRVQIGDTFFEPISWHLATKVLGILCCGLFFGIVGLKIFFSKFQWKIQAELDWRRKHGQAELRDARKHAVEVTLDPETAHPKLCVSDLKTVTHRKAPQEVPHSEKRFTRKSVVASQSFQAGKHYWEVDGGHNKRWRVGVCRDDVDRRKEYVTLSPDHGYWVLRLNGEHLYFTLNPRFISVFPRTPPTKIGVFLDYECGTISFFNINDQSLIYTLTCRFEGLLRPYIEYPSYNEQNGTPIVICPVTQESEKEASWQRASAIPETSNSESSSQATTPFLPRGEM

SEQ ID NO: 73 - RNA, 3583 nt, Homo sapiens
  source 1..3583: mol_type = mRNA
gggactttttggacacccagagaacaggtcccagataccgagtccgcaactccaaacatcgcgattaataggaggcctctggtctctgcctgccctgggtgctcatggaaccagctgctgctctgcacttctccctgccagcctccctcctcctcctcctgctcctcctccttctcagcctgtgtgcactggtctcagcccagtttactgtcgtggggccagctaatcccatcctggccatggtgggagaaaacactacattacgctgccatctgtcacccgagaaaaatgctgaggacatggaggtgcggtggttccggtctcagttctcccccgcagtgtttgtgtataagggtgggagagagagaacagaggagcagatggaggagtaccggggaagaatcacctttgtgagcaaagacatcaacaggggcagcgtggccctggtcatacataacgtcacagcccaggagaatgggatctaccgctgttacttccaagaaggcaggtcctacgatgaggccatcctacgcctcgtggtggcaggccttgggtctaagcccctcattgaaatcaaggcccaagaggatgggagcatctggctggagtgcatatctggagggtggtacccagagcccctcacagtgtggagggacccctacggtgaggttgtgcccgccctgaaggaggtttccatcgctgatgctgacggcctcttcatggtcaccacagctgtgatcatcagagacaagtatgtgaggaatgtgtcctgctctgtcaacaacaccctgctcggccaggagaaggaaactgtcatttttattccagaatcctttatgcccagcgcatctccctggatggtggccctagctgtcatcctgaccgcatctccctggatggtgtccatgactgtcatcctggctgttttcatcatcttcatggctgtcagcatctgttgcatcaagaaacttcaaagggaaaaaaagattctgtcaggggaaaagaaagttgaacaagaggaaaaagaaattgcacagcaacttcaagaagaattgcgatggagaagaacattcttacatgctgctgatgtggtcctggatccagacaccgctcatcccgagctcttcctgtcagaggaccggagaagtgtgaggcggggcccctacaggcagagagtgcctgacaacccagagagattcgacagtcagccttgtgtcctgggatgggagagcttcgcctcagggaaacattactgggaggtggaggtggaaaacgtgatggtgtggactgtgggggtctgcagacacagtgttgagaggaaaggggaggtcctgctgattcctcagaatggcttctggaccctggagatgtttggaaaccaataccgggccctgtcctcccctgagaggattctccctttgaaggagtccctttgccgggtgggcgtcttcctggactatgaagctggagatgtctccttctacaacatgagggacagatcgcacatctacacatgtccccgttcagcctttactgtgcctgtgaggcccttcttcaggttagggtctgatgacagccccatcttcatctgccctgcactcacaggagccagtggggtcatggtgcctgaagagggcctgaaacttcacagagtggggacccaccagagcctatagaatcaattccttggactcacagccatgcagataagccctggccatctcagcagccaccgcacaacccccctaatgaaagacacgccctcctcccctctggtcacgtaagagaacatcttccagctgcctttttcacacccactccagccctctgccccagttttctcctcctcactagtctgtggctttagtagttcctttgcttgtaattatgggatgggatccaggcatagggaactagttgtttcatagctcccagtcaaaaagaaagtgagagaagctgttgggcagcgaacctactgtttaaaatcaggataaccacattaagcccaatatgccagttggcaccagatgctgtggacttggaatgaggccaacagggttcaccaggatgagagaggagagaggaatccacaggaccaccagaagggagagggaaccagatatgcagatcagagatagaggaagtggaaccagagagctgggagggaccaaggttgtaaggatggctaagtcccaccataagagctaaagggtcctgggagatgatggctcatttccacccaaccccaggatttccacagcacacacccacaggcctggacctgggatgaagatgaatgaagaacatggactcatgtggatgtggtttggctcagatgtccctgcaataaacaaggggtcagtacttagtccctgagtgtggttgaggtttgaggtcctggtcgagcagggcagtactggaccaggtctacgtcagcattcaggttcaatggggacaccagtggcttcaaacttcctgatctaattatgtttttagacacttagaagttattgaggactttaaagagcttttgtttatttgggttaatatttatgacatttgacattgaaacaaaaatttaaaatgttatcttttaatttatgttaaaatagcattaataaatcagttataggttaatgtagataggatgttttgtgaaaaagcaatctattgtgtccaaataaaaaaacaaaaagtgtgacactggttaactttttccagatctcatgtctggcttaataagagatatttgtattatcatatctgcctttgtattaaacctattggtatatcataggtcatgttagctcaaaaaaactttactgcacactactgagagaatgagatgaaaaacgattaatgtttcattattattattgtgaaaatattattaacactggggactccttaagagtacatcagagttctctctaggaatcccaaaaccacattttgaaactagaatagtggatcctggaagttaatccatgtgctggttaattttagatgtcaacctgactggattaaggaatacctagacagctggtacaacattatttctgggtgtgtctgtgagtgtgtttccagaagagattggcaagtgagtcagtgggaaattctctccttctgttggctgggtgcccaatacaacaaaaaggcagaggaaaggcaaattcttctctcctctggagctgagacactcttcttcttctgcccttggacatcagaactcctggctctccggcctttgaacttcaggacttgtaccaggaggccctgggttctcaggcctttggctttggactgagagttacacaatcagcttccctggttctgaggctttcagacttaaactgagccatgctaccagcatcccagggtctccagcctacagatgagctgttgtgcgatttcttagcctccataatcacatgagccaatctccttaataaatgcctgctcatagatctgtatctacatctatatctgtatgtgcatctatatctatgcctatatctatatctatatcatattgattttgtctctctggagaaccctgactaataaaatgaggcatctaaaa

SEQ ID NO: 74 - protein, 523 aa, Homo sapiens
MEPAAALHFSLPASLLLLLLLLLLSLCALVSAQFTVVGPANPILAMVGENTTLRCHLSPEKNAEDMEVRWFRSQFSPAVFVYKGGRERTEEQMEEYRGRITFVSKDINRGSVALVIHNVTAQENGIYRCYFQEGRSYDEAILRLVVAGLGSKPLIEIKAQEDGSIWLECISGGWYPEPLTVWRDPYGEVVPALKEVSIADADGLFMVTTAVIIRDKYVRNVSCSVNNTLLGQEKETVIFIPESFMPSASPWMVALAVILTASPWMVSMTVILAVFIIFMAVSICCIKKLQREKKILSGEKKVEQEEKEIAQQLQEELRWRRTFLHAADVVLDPDTAHPELFLSEDRRSVRRGPYRQRVPDNPERFDSQPCVLGWESFASGKHYWEVEVENVMVWTVGVCRHSVERKGEVLLIPQNGFWTLEMFGNQYRALSSPERILPLKESLCRVGVFLDYEAGDVSFYNMRDRSHIYTCPRSAFTVPVRPFFRLGSDDSPIFICPALTGASGVMVPEEGLKLHRVGTHQSL

SEQ ID NO: 75 - RNA, 2945 nt, Homo sapiens
  source 1..2945: mol_type = mRNA
agctttctcacttggtagcagtggcctcttgtgcctttttctccaagatcacccaggctgaagctcctgaggggactcacatcagttatcttgctgctccagaagggtgggagatggcagttttcccaagctccggtctccccagatgtctgctcaccctcattctcctccagctgcccaaactggattcagctccctttgacgtgattggacccccggagcccatcctggccgttgtgggtgaggacgccgagctgccctgtcgcctgtctccgaacgcgagcgccgagcacttggagctacgctggttccgaaagaaggtttcgccggccgtgctggtgcatagggacgggcgcgagcaggaagccgagcagatgcccgagtaccgcgggcgggcgacgctggtccaggacggcatcgccaaggggcgcgtggccttgaggatccgtggcgtcagagtctctgacgacggggagtacacgtgctttttcagggaggatggaagctacgaagaagccctggtgcatctgaaggtggctgctctgggctctgaccctcacatcagtatgcaagttcaagagaatggagaaatctgtctggagtgcacctcagtgggatggtacccagagccccaggtgcagtggagaacttccaagggagagaagtttccatctacatcagagtccaggaatcctgatgaagaaggtttgttcactgtggctgcttcagtgatcatcagagacacttctgcgaaaaatgtgtcctgctacatccagaatctccttcttggccaggagaagaaagtagaaatatccataccagcttcctccctcccaaggctgactccctggatagtggctgtggctgtcatcctgatggttctaggacttctcaccattgggtccatatttttcacttggagactatacaacgaaagacccagagagaggaggaatgaattcagctctaaagagagactcctggaagaactcaaatggaaaaaggctaccttgcatgcagttgatgtgactctggacccagacacagctcatccccacctctttctttatgaggattcaaaatctgttcgactggaagattcacgtcagaaactgcctgagaaaacagagagatttgactcctggccctgtgtgttgggccgtgagaccttcacctcaggaaggcattactgggaggtggaggtgggagacaggactgactgggcaatcggcgtgtgtagggagaatgtgatgaagaaaggatttgaccccatgactcctgagaatgggttctgggctgtagagttgtatggaaatgggtactgggccctcactcctctccggacccctctcccattggcagggcccccacgccgggttgggattttcctagactatgaatcaggagacatctccttctacaacatgaatgatggatctgatatctatactttctccaatgtcactttctctggccccctccggcccttcttttgcctatggtctagcggtaaaaagcccctgaccatctgcccaattgctgatgggcctgagagggtcacagtcattgctaatgcccaggacctttctaaggagatcccattgtcccccatgggggaggactctgcccctagggatgcagacactctccattctaagctaatccctacccaacccagccaaggggcaccttaaggaatatctcagctcatctgttttcctttcctctaacccctctcctccatagccttctgaggcttcacctgctagctttacccagtctgtttcttcctgttgggtggcaattaattaatcctgtgaaggttacattgctgctgctagagagggtggggattgcaccttccaaatctgtttctgtaccaatatttgggggatggaggggtgactcaaactgcttctagtgttctcctaatcccttaagactagaacctataggaaactacttggagcaaactcaaaggacagattagggatcgagattgggtcaggttagcatggggttgtggttgaaatatcttggtatccaggataagggtatgtggaaaaacaggctttaggcaagtggaaaattcaaaatgtgctgtgaaaggacaatctcaggctgaaatcccataaaggaacttggagggaatattatgatggagggaagtgaggtgaatccaggcacatgatgaacacctggctcatccatagagttttcacagcctatatcgcaaattttctaagccacgtcctataggacagaggagactggccccacttctatgggtctgagctgtggaaaagggagagcagagaggaactgagatgagcagggatgaagggtcaggcagaaagcgtgatagaggagagaatttttgacaaaactcaaaagttgtttgcacagctgttctttgtaccctgttcctttctctgcgccctcctgtttctcccttgcctggaagtcattccaccctcaatttgttgatccacaagtttccagttgtcctcttctttttgttatagcatctctctatttcaaagacattcctagaagtcatccttcagtgatatcaccacttgctcagtcaccatctcaaccttatgtcacctcagccctcatctcaatgcccaaaccccttacacacaccttcagttagcttcaactgcctccgtttccacactgtgcacctttcactttccctacccagctttcctacatgctgcctctcctcagggtcccctgaatgctgcatcattgtgttcagtgcagctggactgattgcacctgtgtatttgcccctgagcactttcctttacacatgtggcttgtcttgccaatagactccaggcttataccttccatttccatcgtattctccagtttccaggatagacgttgctcatcgtctttacctaataaataagtttgtctgattgctgaaa

SEQ ID NO: 76 - protein, 526 aa, Homo sapiens
MAVFPSSGLPRCLLTLILLQLPKLDSAPFDVIGPPEPILAVVGEDAELPCRLSPNASAEHLELRWFRKKVSPAVLVHRDGREQEAEQMPEYRGRATLVQDGIAKGRVALRIRGVRVSDDGEYTCFFREDGSYEEALVHLKVAALGSDPHISMQVQENGEICLECTSVGWYPEPQVQWRTSKGEKFPSTSESRNPDEEGLFTVAASVIIRDTSAKNVSCYIQNLLLGQEKKVEISIPASSLPRLTPWIVAVAVILMVLGLLTIGSIFFTWRLYNERPRERRNEFSSKERLLEELKWKKATLHAVDVTLDPDTAHPHLFLYEDSKSVRLEDSRQKLPEKTERFDSWPCVLGRETFTSGRHYWEVEVGDRTDWAIGVCRENVMKKGFDPMTPENGFWAVELYGNGYWALTPLRTPLPLAGPPRRVGIFLDYESGDISFYNMNDGSDIYTFSNVTFSGPLRPFFCLWSSGKKPLTICPIADGPERVTVIANAQDLSKEIPLSPMGEDSAPRDADTLHSKLIPTQPSQGAP

SEQ ID NO: 77 - RNA, 2926 nt, Homo sapiens
  source 1..2926: mol_type = mRNA
acatctgcttcctgtaggccctctgggcagaagcatgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttagcaaccagaggcatcttctggaagatacacttttgtctttgctattatagatgaatatataagcagctgtactctccatcagtgctgcgtgtgtgtgtgtgtgtgtatgtgtgtgtgtgttcagttgagtgaataaatgtcatcctcttctccatcttcatttccttggccttttcgttctattccattttgcattatggcaggcctagggtgagtaacgtggatcttgatcataaatgcaaaattaaaaaatatcttgacctggttttaaatctggcagtttgagcagatcctatgtctctgagagacacattcctcataatggccagcattttgggctacaaggttttgtggttgatgatgaggatggcatgactgcagagccatcctcatctcattttttcacgtcattttcagtaactttcactcattcaaaggcaggttataagtaagtcctggtagcagcctctatggggagatttgagagtgactaaatcttggtatctgccctcaagaacttacagttaaatggggagacaatgttgtcatgaaaaggtattatagtaaggagagaaggagacatacacaggccttcaggaagagacgacagtttggggtgaggtagttggcataggcttatctgtgatgaagtggcctgggagcaccaaggggatgttgaggctagtctgggaggagcaggagttttgtctagggaacttgtaggaaattcttggagctgaaagtcccacaaagaaggccctggcaccaagggagtcagcaaacttcagattttattctctgggcaggcatttcaagtttccttttgctgtgacatactcatccattagacagcctgatacaggcctgtagcctcttccggccgtgtgtgctggggaagccccaggaaacgcacatgcccacacagggagccaagtcgtagcatttgggccttgatctaccttttctgcatcaatacactcttgagcctttgaaaaaagaacgtttcccactaaaaagaaaatgtggatttttaaaatagggactcttcctaggggaaaaaggggggctgggagtgatagagggtttaaaaaataaacaccttcaaactaacttcttcgaacccttttattcactccctgacgactttgtgctggggttggggtaactgaaccgcttatttctgtttaattgcattcaggctggatcttagaagacttttatccttccaccatctctctcagaggaatgagcggggaggttggatttactggtgactgattttctttcatgggccaaggaactgaaagagaatgtgaagcaaggttgtgtcttgcgcatggttaaaaataaagcattgtcctgcttcctaagacttagactggggttgacaattgttttagcaacaagacaattcaactatttctcctaggatttttattattattattttttcacttttctaccaaatgggttacataggaagaatgaactgaaatctgtccagagctccaagtcctttggaagaaagattagatgaacgtaaaaatgttgttgtttgctgtggcagtttacagcatttttcttgcaaaattagtgcaaatctgttggaaatagaacacaattcacaaattggaagtgaactaaaatgtaatgacgaaaagggagtagtgttttgatttggaggaggtgtatattcggcagaggttggactgagagttgggtgttatttaacataattatggtaattgggaaacatttataaacactattgggatggtgataaaatacaaaagggcctatagatgttagaaatgggtcaggttactgaaatgggattcaatttgaaaaaaatttttttaaatagaactcactgaactagattctcctctgagaaccagagaagaccatttcatagttggattcctggagacatgcgctatccaccacgtagccactttccacatgtggccatcaaccacttaagatggggttagtttaaatcaagatgtgctgttataattggtataagcataaaatcacactagattctggagatttaatatgaataataagaatactatttcagtagttttggtatattgtgtgtcaaaaatgataatattttggatgtattgggtgaaataaaatattaacatta

SEQ ID NO: 78 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 79 - RNA, 1086 nt, Homo sapiens
  source 1..1086: mol_type = mRNA
gaacttagggctgcttgtggctgggcactcgcgcagaggccggcccgacgagccatggttgctgggagcgacgcggggcgggccctgggggtcctcagcgtggtctgcctgctgcactgctttggtttcatcagctgtttttcccaacaaatatatggtgttgtgtatgggaatgtaactttccatgtaccaagcaatgtgcctttaaaagaggtcctatggaaaaaacaaaaggataaagttgcagaactggaaaattctgaattcagagctttctcatcttttaaaaatagggtttatttagacactgtgtcaggtagcctcactatctacaacttaacatcatcagatgaagatgagtatgaaatggaatcgccaaatattactgataccatgaagttctttctttatgtgcttgagtctcttccatctcccacactaacttgtgcattgactaatggaagcattgaagtccaatgcatgataccagagcattacaacagccatcgaggacttataatgtactcatgggattgtcctatggagcaatgtaaacgtaactcaaccagtatatattttaagatggaaaatgatcttccacaaaaaatacagtgtactcttagcaatccattatttaatacaacatcatcaatcattttgacaacctgtatcccaagcagcggtcattcaagacacagatatgcacttatacccataccattagcagtaattacaacatgtattgtgctgtatatgaatggtattctgaaatgtgacagaaaaccagacagaaccaactccaattgattggtaacagaagatgaagacaacagcataactaaattattttaaaaactaaaaagccatctgatttctcatttgagtattacaatttttgaacaactgttggaaatgtaacttgaagcagctgctttaagaagaaatacccactaacaaagaacaagcattagttttggctgtcatcaacttattatatgactaggtgcttgctttttttgtcagtaaattgtttttactgatgatgtagatacttttgtaaataaatgtaaatatgtacacaagtga

SEQ ID NO: 80 - protein, 250 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVLESLPSPTLTCALTNGSIEVQCMIPEHYNSHRGLIMYSWDCPMEQCKRNSTSIYFKMENDLPQKIQCTLSNPLFNTTSSIILTTCIPSSGHSRHRYALIPIPLAVITTCIVLYMNGILKCDRKPDRTNSN

SEQ ID NO: 81 - RNA, 3749 nt, Homo sapiens
  source 1..3749: mol_type = mRNA
agacagcctctgctgcatgacacgaagcttgcttctgcctggcatctgtgagcagctgccaggctccggccaggatcccttccttctcctcattggctgatggatcccaaggggctcctctccttgaccttcgtgctgtttctctccctggcttttggggcaagctacggaacaggtgggcgcatgatgaactgcccaaagattctccggcagttgggaagcaaagtgctgctgcccctgacatatgaaaggataaataagagcatgaacaaaagcatccacattgtcgtcacaatggcaaaatcactggagaacagtgtcgagaacaaaatagtgtctcttgatccatccgaagcaggccctccacgttatctaggagatcgctacaagttttatctggagaatctcaccctggggatacgggaaagcaggaaggaggatgagggatggtaccttatgaccctggagaaaaatgtttcagttcagcgcttttgcctgcagttgaggctttatgagcaggtctccactccagaaattaaagttttaaacaagacccaggagaacgggacctgcaccttgatactgggctgcacagtggagaagggggaccatgtggcttacagctggagtgaaaaggcgggcacccacccactgaacccagccaacagctcccacctcctgtccctcaccctcggcccccagcatgctgacaatatctacatctgcaccgtgagcaaccctatcagcaacaattcccagaccttcagcccgtggcccggatgcaggacagacccctcagaaacaaaaccatgggcagtgtatgctgggctgttagggggtgtcatcatgattctcatcatggtggtaatactacagttgagaagaagaggtaaaacgaaccattaccagacaacagtggaaaaaaaaagccttacgatctatgcccaagtccagaaaccaggtcctcttcagaagaaacttgactccttcccagctcaggacccttgcaccaccatatatgttgctgccacagagcctgtcccagagtctgtccaggaaacaaattccatcacagtctatgctagtgtgacacttccagagagctgacaccagagaccaacaaagggactttctgaaggaaaatggaaaaaccaaaatgaacactgaacttggccacaggccccaagtttcctctggcagacatgctgcacgtctgtacccttctcagatcaactccctggtgatgtttcttccacatacatctgtgaaatgaacaaggaagtgaggcttcccaagaatttagcttgctgtgcagtggctgcaggcgcagaacagagcgttacttgataacagcgttccatctttgtgttgtagcagatgaaatggacagtaatgtgagttcagactttgggcatcttgctcttggctggaactggataataaaaatcagactgaaagccaggacatctgagtacctatctcacacactggaccaccagtcacaaagtctggaaaagtttacattttggctatctttactttgttctgggagctgatcatgataacctgcagacctgatcaagcctctgtgcctcagtttctctctcaggataaagagtgaatagaggctgaagggtgaatttcttattatacataaaacactctgatattattgtataaaggaagctaagaatattattttatttgcaaaacccagaagctaaaaagtcaataaacagaaagaatgattttgagatctctgagttttgaacagtggactggaaaccatgtaagagccttaaaagtacagttctgtgcaaatggcattcagttttaaagaaaaacgtagcaaatgtttgatggtgctgttacaaaggagcttggaatactcagaggaacttgtcccatggtgatttttcacttctcaaaatgatgtttaaatcccagttctctgttgattcccttgaacaacaaacctggaacctcagctaagactctctgtgaccagattctgaacctcttatatccagggcttcaaggggtattgcaggtcaaggtctttcctaggcactttctactccctgcatacctctcctcacactaaatttatcctctagtagaaaattaagttattttggtctaacagcttcaaatctttgaatgctcaataacttattttgcaagctgcaggcagaaagagactttttaagtaaagtcctttgttttttcctattctctgcttttagacaggctgtcctcaatttaagccctgctttttcttattgtttcttatataaacttggtaagtactgtaagaaacagccactatcataccattgcataataaggagcaccaacttcccagctcaaaactcaggtccttattgccttgtatcttacctcctctatgaggtcaattcacattgtaagcctgttgcttagtgcatctcgtttcctggtaccagcttctttaatagagttcttagttgcaatcaacagaagctggctttggcttttttatgtagaaaaggaacctattgaaaagatactgattggttccaataactgctagaagtttctgcaaaaccatgctttgaaagtgagcaggaaaagaagagactaggctgtggctgggagcacagccaaaattacaaaaccagcccagggatgatgatcctgttcatgcacagccactgtccccagcactaggcacagactctaccactgcctcactgtctctgctggacttggaaacttgatattactgttactgctgcactgtctgccatgaaaatgaattctccagggtcccttcttcatcctttcatctctagcttataattcaaagtctgggattgagtggccaatcctaggtcacatgtccatgtcctatctccaaggggggctgggaattgaatatctggcattttccactttcacttcttatgaattaaggaattctacaaataatagaagtgggattcaggtggtaggcagacaaaaaagcctcacaattatccactacgccacccttgtataaccttaccctcattcactgtctactctcaaaactgtggagctactaatgaagatttgtaaacccgggcttatgagcacccattcctttactacaactcagattgctctagaagctcagttcccagcacttggatttttccagtagctgaattctacctgaaggaagggcagaaacaaagggtgaagaagaggctatcacttccaagtatcctgcacccctgggctcaagacctcactggggagggagtcttttgggccacccaccaaacagcactggcattatgcctctcaccctagaccatggttacacgtggtaaaacaaccccttctggtgatacattcacaactctctagtttcccccaaatggcactatggggagcgggagcttgccttttcctcagacttaaaacaataagttttccccgtgtttcccctctaatgctgttttcttttgaccaagcatgtctgaattctagagaagtcaggaggaacacacccattctcggtttgaagggactgatgttctgaagtacaactgggcacagtcccaggctcttcaggacgcttcctccattcacacagcggggatgtgattgttacagcgggtggtgtgtgctggctgagaagccactgtgaattgattcttcttctgaagtttatgtttctactttttggaaatgaataaattacagccagtccatcaaggaaa

SEQ ID NO: 82 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 83 - RNA, 4721 nt, Homo sapiens
  source 1..4721: mol_type = mRNA
acacttcgggttcctcggggaggaggggctggaaccctagcccatcgtcaggacaaagatgctcaggctgctcttggctctcaacttattcccttcaattcaagtaacaggaaacaagattttggtgaagcagtcgcccatgcttgtagcgtacgacaatgcggtcaaccttagctgcaagtattcctacaatctcttctcaagggagttccgggcatcccttcacaaaggactggatagtgctgtggaagtctgtgttgtatatgggaattactcccagcagcttcaggtttactcaaaaacggggttcaactgtgatgggaaattgggcaatgaatcagtgacattctacctccagaatttgtatgttaaccaaacagatatttacttctgcaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctgacacggacgcctatccagaagccagccggctggcagcccccatctgctcaatatcactgctctggataggaaatgaccgccatctccagccggccacctcaggcccctgttgggccaccaatgccaatttttctcgagtgactagaccaaatatcaagatcattttgagactctgaaatgaagtaaaagagatttcctgtgacaggccaagtcttacagtgccatggcccacattccaacttaccatgtacttagtgacttgactgagaagttagggtagaaaacaaaaagggagtggattctgggagcctcttccctttctcactcacctgcacatctcagtcaagcaaagtgtggtatccacagacattttagttgcagaagaaaggctaggaaatcattccttttggttaaatgggtgtttaatcttttggttagtgggttaaacggggtaagttagagtagggggagggataggaagacatatttaaaaaccattaaaacactgtctcccactcatgaaatgagccacgtagttcctatttaatgctgttttcctttagtttagaaatacatagacattgtcttttatgaattctgatcatatttagtcattttgaccaaatgagggatttggtcaaatgagggattccctcaaagcaatatcaggtaaaccaagttgctttcctcactccctgtcatgagacttcagtgttaatgttcacaatatactttcgaaagaataaaatagttctcctacatgaagaaagaatatgtcaggaaataaggtcactttatgtcaaaattatttgagtactatgggacctggcgcagtggctcatgcttgtaatcccagcactttgggaggccgaggtgggcagatcacttgagatcaggaccagcctggtcaagatggtgaaactccgtctgtactaaaaatacaaaatttagcttggcctggtggcaggcacctgtaatcccagctgcccaagaggctgaggcatgagaatcgcttgaacctggcaggcggaggttgcagtgagccgagatagtgccacagctctccagcctgggcgacagagtgagactccatctcaaacaacaacaacaacaacaacaacaacaacaaaccacaaaattatttgagtactgtgaaggattatttgtctaacagttcattccaatcagaccaggtaggagctttcctgtttcatatgtttcagggttgcacagttggtctctttaatgtcggtgtggagatccaaagtgggttgtggaaagagcgtccataggagaagtgagaatactgtgaaaaagggatgttagcattcattagagtatgaggatgagtcccaagaaggttctttggaaggaggacgaatagaatggagtaatgaaattcttgccatgtgctgaggagatagccagcattaggtgacaatcttccagaagtggtcaggcagaaggtgccctggtgagagctcctttacagggactttatgtggtttagggctcagagctccaaaactctgggctcagctgctcctgtaccttggaggtccattcacatgggaaagtattttggaatgtgtcttttgaagagagcatcagagttcttaagggactgggtaaggcctgaccctgaaatgaccatggatatttttctacctacagtttgagtcaactagaatatgcctggggaccttgaagaatggcccttcagtggccctcaccatttgttcatgcttcagttaattcaggtgttgaaggagcttaggttttagaggcacgtagacttggttcaagtctcgttagtagttgaatagcctcaggcaagtcactgcccacctaagatgatggttcttcaactataaaatggagataatggttacaaatgtctcttcctatagtataatctccataagggcatggcccaagtctgtctttgactctgcctatccctgacatttagtagcatgcccgacatacaatgttagctattggtattattgccatatagataaattatgtataaaaattaaactgggcaatagcctaagaaggggggaatattgtaacacaaatttaaacccactacgcagggatgaggtgctataatatgaggaccttttaacttccatcattttcctgtttcttgaaatagtttatcttgtaatgaaatataaggcacctcccacttttatgtatagaaagaggtcttttaatttttttttaatgtgagaaggaagggaggagtaggaatcttgagattccagatcgaaaatactgtactttggttgatttttaagtgggcttccattccatggatttaatcagtcccaagaagatcaaactcagcagtacttgggtgctgaagaactgttggatttaccctggcacgtgtgccacttgccagcttcttgggcacacagagttcttcaatccaagttatcagattgtatttgaaaatgacagagctggagagttttttgaaatggcagtggcaaataaataaatacttttttttaaatggaaagacttgatctatggtaataaatgattttgttttctgactggaaaaataggcctactaaagatgaatcacacttgagatgtttcttactcactctgcacagaaacaaagaagaaatgttatacagggaagtccgttttcactattagtatgaaccaagaaatggttcaaaaacagtggtaggagcaatgctttcatagtttcagatatggtagttatgaagaaaacaatgtcatttgctgctattattgtaagagtcttataattaatggtactcctataatttttgattgtgagctcacctatttgggttaagcatgccaatttaaagagaccaagtgtatgtacattatgttctacatattcagtgataaaattactaaactactatatgtctgctttaaatttgtactttaatattgtcttttggtattaagaaagatatgctttcagaatagatatgcttcgctttggcaaggaatttggatagaacttgctatttaaaagaggtgtggggtaaatccttgtataaatctccagtttagccttttttgaaaaagctagactttcaaatactaatttcacttcaagcagggtacgtttctggtttgtttgcttgacttcagtcacaatttcttatcagaccaatggctgacctctttgagatgtcaggctaggcttacctatgtgttctgtgtcatgtgaatgctgagaagtttgacagagatccaacttcagccttgaccccatcagtccctcgggttaactaactgagccaccggtcctcatggctattttaatgagggtattgatggttaaatgcatgtctgatcccttatcccagccatttgcactgccagctgggaactataccagacctggatactgatcccaaagtgttaaattcaactacatgctggagattagagatggtgccaataaaggacccagaaccaggatcttgattgctatagacttattaataatccaggtcaaagagagtgacacacactctctcaagacctggggtgagggagtctgtgttatctgcaaggccatttgaggctcagaaagtctctctttcctatagatatatgcatactttctgacatataggaatgtatcaggaatactcaaccatcacaggcatgttcctacctcagggcctttacatgtcctgtttactctgtctagaatgtccttctgtagatgacctggcttgcctcgtcacccttcaggtccttgctcaagtgtcatcttctcccctagttaaactaccccacaccctgtctgctttccttgcttatttttctccatagcattttaccatctcttacattagacatttttcttatttatttgtagtttataagcttcatgaggcaagtaactttgctttgtttcttgctgtatctccagtgcccagagcagtgcctggtatataataaatatttattgactgagtgaa

SEQ ID NO: 84 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 85 - RNA, 1282 nt, Homo sapiens
  source 1..1282: mol_type = mRNA
ggaagtctgtcaactgggagggggagaggggggtgatgggccaggaatggggtccccgggcatggtgctgggcctcctggtgcagatctgggccctgcaagaagcctcaagcctgagcgtgcagcaggggcccaacttgctgcaggtgaggcagggcagtcaggcgaccctggtctgccaggtggaccaggccacagcctgggaacggctccgtgttaagtggacaaaggatggggccatcctgtgtcaaccgtacatcaccaacggcagcctcagcctgggggtctgcgggccccagggacggctctcctggcaggcacccagccatctcaccctgcagctggaccctgtgagcctcaaccacagcggggcgtacgtgtgctgggcggccgtagagattcctgagttggaggaggctgagggcaacataacaaggctctttgtggacccagatgaccccacacagaacagaaaccggatcgcaagcttcccaggattcctcttcgtgctgctgggggtgggaagcatgggtgtggctgcgatcgtgtggggtgcctggttctggggccgccgcagctgccagcaaagggactcaggtaacagcccaggaaatgcattctacagcaacgtcctataccggccccggggggccccaaagaagagtgaggactgctctggagaggggaaggaccagaggggccagagcatttattcaacctccttcccgcaaccggccccccgccagccgcacctggcgtcaagaccctgccccagcccgagaccctgccccagccccaggcccggccaccccgtctctatggtcagggtctctcctagaccaagccccacccagcagccgaggccaaaagggttccccaaagtgggagaggagtgagagatcccaggagacctcaacaggaccccacccataggtacacacaaaaaaggggggatcgaggccagacacggtggctcacgcctgtaatcccagcagtttgggaagccgaggcgggtggaacacttgaggtcaggggtttgagaccagcctggcttgaacctgggaggcggaggttgcagtgagccgagattgcgccactgcactccagcctgggcgacagagtgagactccgtctcaaaaaaaacaaaaagcaggaggattgggagcctgtcagccccatcctgagaccccgtcctcatttctgtaatgatggatctcgctcccactttcccccaagaacctaataaaggcttgtgaagaaaaagcaaaaaaaaaaaaaaaaaa

SEQ ID NO: 86 - protein, 282 aa, Homo sapiens
MGSPGMVLGLLVQIWALQEASSLSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 87 - RNA, 1643 nt, Homo sapiens
  source 1..1643: mol_type = mRNA
agtctcacttcagttccttttgcatgaagagctcagaatcaaaagaggaaaccaacccctaagatgagctttccatgtaaatttgtagccagcttccttctgattttcaatgtttcttccaaaggtgcagtctccaaagagattacgaatgccttggaaacctggggtgccttgggtcaggacatcaacttggacattcctagttttcaaatgagtgatgatattgacgatataaaatgggaaaaaacttcagacaagaaaaagattgcacaattcagaaaagagaaagagactttcaaggaaaaagatacatataagctatttaaaaatggaactctgaaaattaagcatctgaagaccgatgatcaggatatctacaaggtatcaatatatgatacaaaaggaaaaaatgtgttggaaaaaatatttgatttgaagattcaagagagggtctcaaaaccaaagatctcctggacttgtatcaacacaaccctgacctgtgaggtaatgaatggaactgaccccgaattaaacctgtatcaagatgggaaacatctaaaactttctcagagggtcatcacacacaagtggaccaccagcctgagtgcaaaattcaagtgcacagcagggaacaaagtcagcaaggaatccagtgtcgagcctgtcagctgtccaggaggcagcatccttggccagagtaatgggctctctgcctggacccctcccagccatcccacttctcttccttttgcagagaaaggtctggacatctatctcatcattggcatatgtggaggaggcagcctcttgatggtctttgtggcactgctcgttttctatatcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaattaaaaaagatagaaactgtctttttcaataaaaagcactgtggatttctgccctcctgatgtgcatatccgtacttccatgaggtgttttctgtgtgcagaacattgtcacctcctgaggctgtgggccacagccacctctgcatcttcgaactcagccatgtggtcaacatctggagtttttggtctcctcagagagctccatcacaccagtaaggagaagcaatataagtgtgattgcaagaatggtagaggaccgagcacagaaatcttagagatttcttgtcccctctcaggtcatgtgtagatgcgataaatcaagtgattggtgtgcctgggtctcactacaagcagcctatctgcttaagagactctggagtttcttatgtgccctggtggacacttgcccaccatcctgtgagtaaaagtgaaataaaagctttgactaga

SEQ ID NO: 88 - protein, 377 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPGGSILGQSNGLSAWTPPSHPTSLPFAEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 89 - RNA, 1097 nt, Homo sapiens
  source 1..1097: mol_type = mRNA
ctttttctagccaggctctcaactgtctcctgcgttgctgggaagttctggaaggaagcatgtgctccagaggttgggattcgtgtctggctctggaattgctactgctgcctctgtcactcctggtgaccagcattcaaggtcacttggtacatatgaccgtggtctccggcagcaacgtgactctgaacatctctgagagcctgcctgagaactacaaacaactaacctggttttatactttcgaccagaagattgtagaatgggattccagaaaatctaagtactttgaatccaaatttaaaggcagggtcagacttgatcctcagagtggcgcactgtacatctctaaggtccagaaagaggacaacagcacctacatcatgagggtgttgaaaaagactgggaatgagcaagaatggaagatcaagctgcaagtgcttgaccctgtacccaagcctgtcatcaaaattgagaagatagaagacatggatgacaactgttatctgaaactgtcatgtgtgatacctggcgagtctgtaaactacacctggtatggggacaaaaggcccttcccaaaggagctccagaacagtgtgcttgaaaccacccttatgccacataattactccaggtgttatacttgccaagtcagcaattctgtgagcagcaagaatggcacggtctgcctcagtccaccctgtaccctggcccggtcctttggagtagaatggattgcaagttggctagtggtcacggtgcccaccattcttggcctgttacttacctgagatgagctcttttaactcaagcgaaacttcaaggccagaagatcttgcctgttggtgatcatgctcctcaccaggacagagactgtataggctgaccagaagcatgctgctgaattatcaacgaggattttcaagttaacttttaaatactggttattatttaattttatatccctttgttgttttctagtacacagagatatagagatacacatgcttttttcccacccaaaattgtgacaacattatgtgaatgttttattattttttaaaataaacatttgatataattgtcaattaactgaa

SEQ ID NO: 90 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 91 - RNA, 12123 nt, Homo sapiens
  source 1..12123: mol_type = mRNA
gcagatgggaagaagcgttagagcgagcagcactcacatctcaagaaccagcctttcaaacagtttccagagatggattatcctactttacttttggctcttcttcatgtatacagagctctatgtgaagaggtgctttggcatacatcagttccctttgccgagaacatgtctctagaatgtgtgtatccatcaatgggcatcttaacacaggtggagtggttcaagatcgggacccagcaggattccatagccattttcagccctactcatggcatggtcataaggaagccctatgctgagagggtttactttttgaattcaacgatggcttccaataacatgactcttttctttcggaatgcctctgaagatgatgttggctactattcctgctctctttacacttacccacagggaacttggcagaaggtgatacaggtggttcagtcagatagttttgaggcagctgtgccatcaaatagccacattgtttcggaacctggaaagaatgtcacactcacttgtcagcctcagatgacgtggcctgtgcaggcagtgaggtgggaaaagatccagccccgtcagatcgacctcttaacttactgcaacttggtccatggcagaaatttcacctccaagttcccaagacaaatagtgagcaactgcagccacggaaggtggagcgtcatcgtcatccccgatgtcacagtctcagactcggggctttaccgctgctacttgcaggccagcgcaggagaaaacgaaaccttcgtgatgagattgactgtagccgagggtaaaaccgataaccaatataccctctttgtggctggagggacagttttattgttgttgtttgttatctcaattaccaccatcattgtcattttccttaacagaaggagaaggagagagagaagagatctatttacagagtcctgggatacacagaaggcacccaataactatagaagtcccatctctaccagtcaacctaccaatcaatccatggatgatacaagagaggatatttatgtcaactatccaaccttctctcgcagaccaaagactagagtttaagcttattcttgacatgagtgcattagtaatgactcttatgtactcatgcatggatctttatgcaatttttttccactacccaaggtctaccttagatactagttgtctgaattgagttactttgataggaaaaatacttcattacctaaaatcatttttcatagaactgtttcagaaaacctgactctaactggtttatatacaaaagaaaacttactgtatcatataacagaatgatccaggggagattaagctttgggcaagggctatttaccagggcttaaatgttgtgtctagaattaagtatgggcataaactggcttctgaatccctttccagagtgttggatccatttccctggtcttggcctcactctcatgcaggctttcctcttgtgttggcaagatggctgccaactcttggcaattcatacatccttgtttctgtctggtagagagtttgcttctcaaatggagcaaacaaatttgattattttttcattgttaaataggcaacatgaccagaaaggatggaatggcttaagtaaactaagggttcacttctagagctgagaagcagggtcaaagcacaatactgggcaattcagagcatggttagaagaggaaaggggagtctcaaagctggagagtttaccaacaaatattgactgcagtgattaaccaagacatttttgttaactaaaaagtgaaatatgggatggattctagaaatggggtatctctgtccatacttctagaatccactctatcagcatagtccagaagaatacctggcagtagaagaaatgaatattcaagaggaagataaatgcgagagggcaatcctttactattctcatatttatttatctctcattctgtatagaattcttgccgccatcccaggtctagccttaggagcaaatgtagtagatagtcgaataataaataacttaatgttttggacatattttgtctacttttgagaattatttttaatatgtaaattctctcaaaagggtcaggcacctagttattattttttaatgattatgtgaaagttgaatataatataccactaaaagtgacagttgaaagtggtggcataggacggtagggtagaaatttgggagggaaaaaagaaattgggagggtacaggcaacaggagaaaggaatcaaaccacagaaaaatacaaagggaaacttctgcttcactattcagacaaagacagccctaatgacatcaccaacagtcaaagcaattagagaccatacctaatattgtttaaattctagatgtaggctaacaatgaaaagtatttgccaaactgaataaaactgtcatggttaccttgaaaggacaatggttattgttaaatatagtgatcattcatgtctaaaagattcattatttatctctaaagatttctaaagaccaccatctagaaaagattcattatgaaggctgtatttaaatatcaaagttgtggacttcatgataatcttaaataaagcaaatccaaattctcctgttgcctagacagattctaagatgtaatttacacttttaagctaattagtgagtattttatgattttagccttaaacaccatgtatgccaaataatgcacttgttttgtgaattacagaaatggtaagtgcccacatttctgtgaattataaaatttgtgagtttcttttaacccttttcaggagtgaaaaaataaaaacgaccatttcctggttgtgcttaagtatatgcaagaagggtaaactctcatttttattatgtttgcttaaagatctttttatacctggattcatgaaatgtttccacaaatatattagtgtaacaaacttgaaaggcagtttacaagaaagcactctactatcagatcaatcaaagattctgtgagtgaatttattggtttgcatggtgaagcaagcttagcatcaattaaaaggtaaataatttcttttctgaatggtaaagacaatcaaaatattactttctggaaaactccaataaccaaattctcaatgattagtgtatgtgagcaggaaaacatttttacagttgtagtatggggaaatataaatccaattttaagagagaaaattatgactgggtgtggaagggacagtatagtcagataccattgtcatggtggtttttactgggaacttcatgaaagacttttgtagcaaaccactgcagtattgcaaagcctccagaacatttggaacttgtctctttttccttgtgtgtgtttgtgtttttggtctctcattcaaaatattgatgagaactatttactctgtcctttcttctctatatattcttcctctacagagtgtagggttttttcaggaatttggagccatctgaagtcctcccaaaaattctctgacgtcttctgatgctcctgttataccctcaggggtaatgcttgtgaaattccattcattcattttctttctctggacatctttacttaccaaagcactttcattgtcatctttttaacatcattcttaattcgtgatagttttgggactctccctagtgtatgtttctccccctctactcttttgcacctatgattctgattgttactaagaaagcagatgaaaaacagatccacagaataaacgatcagaattccagtaaattctattttaaatacagatactttttacaagttgctgctttggaagcaaaatgcttcttaagttttacatatatatatatatatatacatatatatatacacatataatttatatcgatggataatacattaagaatctatgcttcctttgaatgccattaatatttatgttaaagtaaccaatgaaaggaaattactttgttataataagataggaagacttgttaatggagtacacagttttgtcagggaaagaacacatcttattgaactatgatgactatgcattgactatattattataagagataccttcaaactttatttaaagaactttaggtataatatgttgagaaaataaaatagaaatttcatttacttgtaatcatgcttaaaatgggaggcaggtaggtgaagatataatttttagtaaaaactccaatttatgttttaagtaattcagtgtattactaaaatactatatatataaacttaaaatacatgggttatcaatttaaaagacaaagtaagtaaaaatacttttagtaggcattcgtggattgtgaacatccaagttatattggtttgtatagaatggcattaagtaaaaattacagctgtataacagtagttttctaaattgagagagtccacattgtaattagagatcactgtgaccaaaatgcttctccttgatttataatgatgtactgtattttgtactgcttatatgaaatttcagcaagattgacgatattataaagatgcttataaagtgtaagtggagacgctaaattgtgagtacaaagtttctttttcacaacagtgataagaaaatatctttaaaaaatataagacaatataaacatgtcatcattagtttagctactattaaaatgtaacatctagaaagtactgatctccaccttcagacttctgtataagtatattttttcactgatctgttcattagagttcttccagccaagactctgggctcttaaaacatgtatctgaaaactaaaaacaagttaatttttttaaaagcttctctatttctagtgattcaataggtagaaaaatagcttctagaattaactgcaatgctttctaaggaaattttataaatcctcaaggtcggtttacacatatttttccagattcagagcactaactatcttgtaagatgtaagaaaaggtccatttggaagtatgagtaataaatgtctgggataattctggtttatttcgtattatccttgttagaataagttatatggtcaacctgttcagaacactttttctagtgttagtgtgtacttttggatttttggttcttgtagggtatagaaatattttcctttgtcttgtattctgttgttttgaatgaataaaacacaatgtttcacgatcactactttcatttgccatggagaaatagcagggaaaaatttctacagaataaaattaactgatgaattacatgcagaaaaaattcaaatcaatgatacattgtaatttttatctcaatgcaatgttctttgtattttattttattattatttttttgagacggagtttcacttttgttgcccgggctggagtgcaatggcacaatctcggctcaccacaacctctgcctcccggattcaagtgattctcctgcctcagcctcctgaatagctgggattacaggcatatgccaacatgcctggctaattttgtatttttagtggagacggggtttctccacgttggtcagacttgtcttgaactctggacctcaggtgatccacctgcctcagcctcctaaattgctgggattacaggcatgagcgaccactcctggccttgttctttgtattttataagtgcatgtagtgcaaagggtcaaagggctttacaggttttttgtttgtttgtttttgtttttcccgaaacatagtagtcccttgcccttcctcatttttgttaccttgagacaacaaattttactacttctaactcattattttatttatgttcacttttctgaatagcatgcttatgacactaatactttttttttcaattttagacattcattattcatttagatgtctttctctccccaaactcaccacataaaatactcttctcatgtctctttcagaaatatttgtattaaaatatgattatatcaatatttggcatttatttcttatgaccttgccagtactcttagttaaactacatggtaaaaatgattttgctttccctcctacataactttttttccacctagagctaataattgtcattctggggactgactttttctgtatttaccataaattgacctgaaactcccctgtgatgcagcaggaattctaccaacgtcaacttccttagaaagactccattagaagcttgacttggggctagaaggagaggcacacaactgccatcctggtgtctcccttcatccagaaaaagggggaggaatacatgaaacctagaatccactctaaaacattttccagaacaaaaggacatgtgtttccgtgttgtaaatgtttaacgagtgcccataacaaggaataataagtctattatgtttgcttttgtgtctgtaaaagttgggggtattggttgtaagcacgaaaacagatactgactgttgaagaaaaaaaaaaatacgaggtcaggagtttgagaccaacttggccaatatggtgaaaccctgtcttagtaaaaatagaaaaattagccaggcctggtggcacgcacctgtagtcccagctacttgggaggctgaggcagaagaatcgcttgaacccgggaggcagaggttgcagtgagccaagatcgcaccactgcactccaccctgggcaacagagcgagactccgtctcaaaaaaaaaaaaaaaaaaaaaaaaaaaaagttaagtatttgaacataggggtggctcatagaattcccaggacacccgatggagtaggcttgcaaaacacaacatgtggcaactccagtgggaaacgaggcaggaaacactcgtttcctgcagaaagcaacaatttgggcttcgataccctccctagaacacagggcagtgaatctgagcagcatcagtaccccacgttcggatgagtcctgagcccctatttttattcactgacttattccaaaatcagtgtctcttaaatatatctggaaggcagcagcttgtatctcccccttcagcttccatagtggcagtcagggtacaacttactttccaaacagaacacactgcgacattccctccaggctcgttgaagaacttcaactgacaaatgtccctcctcgaccagatgatagttttcttaaaggcagggtttaatatacccttttataaatgtttcaaggccctgtgtaatacctgagtttattccagatgtaactaaatatatccaagattgttttaaaataaattgctgaaaaaacaaataaatacagttagtatctatatcaatattctcagttggcagttttgcaataatggccgatagttcatttttagtaacactattgacattgcatttggatattagggtttactaatcatccgcatgtatacattgcatatttttctagactttaactttattcaaatctattgatttttaaacctgcaacttatgtctagacacaggtatacctttacaagaactaccattttttttggtaacatactacctccaaaatttcaagtaagaagttgatttttgtccatttttaaatggaaaacttgtaatcaaaatgccacaaaattatactgtgtatcatttgacctatagaaaccaatattattacaggaagaaagcagagccaatcttctacctgtggtcaaataagtggaggccctttctagactaagttctcatgagtttaaaataccaagcataagttctccaaattcctgaaaaggaagccttgtgttgtattgcccagccatatttgtaagacataaaaataaaacttgagaagaagctatgataacttactttcttcattcttcaaaatttacataatctcaactgattttatgtttttatgaaaatgcattcttaagatatatccttattcaatcatgtattcattacatcctttatgccaggtatccaaaagtacttacagtgactaagaccattattctttgatcagctgcctgagtaagactttgagctctccaatatactctcagtgatactaagttttctgagtaacagctttggatgtggcttcagttgagctgatttatcccacactttatttttatcgtataatggtcctcagaagcaaattttgattttagctcacataaaaaatgtacaaagaaatgtaatggctcagtagcttctagagatagagattactcttctaacctttctgtaattttgtatgtctattttataattctttcaatgtctaatgaatagctatctttttttgagacggagtctcgctctgtcgcccaggctggagtgcagtggtgcgacctcggctcaccgcaagctgcgtcttccaggttcacgccattctcctgcctcagcctcccgagtagctgggacttcaggcgcccaccaccatgcccagctaatttttttgtatttttagtagagacggggtttcaccgtgttagccagggtggtctcgatctcctgacctcgtgatccgcccgcctcggcctcccaacgtgctgggattacaggagtgagccaccgcgcccggcctccttagtttcttaaggtggaagcctagattattgattttatatgttgttttcttttccaatagtggcacttaatgctataaatttcactttgttccacaagttttggtaagctctatttttattttcatttagtccaaaatattttaaaatttcttttgatatttcttctttgagccatgaattatttacaatgtgttgtttaatctctatatattttgggatttttctactttatatctcttacagatttctaacttaatttcatcatgttttaaaaacattctttgtataatttctattcttttaaatttttcaggtgtattttatggcccagaatatggtctatcttgtagaatgtttcatgtgatcttaagaagaatgttcattctgctgttgagtgtaatattctacaaatgtccattagattaaactgattgataccaccgttcagattatctatatcctttctgattttccctcttcttgatctatcacatactgacagatcaagtgatcaagtctcgttaaagactgcaagtaaaatagtggatttttctatttctccttgcagttttgttagtttttgtctcatgtatcttgatactcttgttagtacatatactttcagaatcgttaggttttcttggagaattgacccctttaccacatgtaatgtcccttttattcttgataatctttcttgttctgtctgctttttctgatattaacataactttcagttttttaaaaaattaacattagcatctcacatctttatccttttaattttaaattatctaaatatttatatttaatgtgcctttcttatagacaatgtatagttgcgtctatttgtaatttccccacttttcttacttaaaaatgttgtagatatataggagttgtatatatttggggggtacatgtgatgttttgatacctgtatacaatatgtaatgatcatattgggtaatcgtgatatctgtcacctctaacattcatcttttttgtgtgtttaaacccaccacttctaattggtacatttagattattcaaatttaagtgattattgatatagttggattaatatctactatgtttgtaacttttctatccttgcactcgttctttcttttttatcctcctttttctgtgttctctgattttaactggggtttttacatgatttaattttctctcgtggcatatctttcattgatcaacctaggtttttctccttttcccctcttttttttggtatttattctatttagtgttatctgagctacctgagttggtgtctatcactaattttggcaagttcccagacgttattacttctaacattcttttgctccattctttcttcttcttcaattattccatagtcttgaatattctgggtttttcccactctttgaattttagtttgaaaagtttctattggcctagcttcaaagtcattcattcttccttcggggttccaagtcaactgataattgcatcaaagatatccttcctttctattactatgttttttattgctaccatttcttttttattccttcttagtgtttccatctttcttcttacattatccatctgttgtctatttttttcatgagagctcttaacatattaatgataagttccatgtctgataattctgacacgtgtcatgtctctatctggttccaatgattgctttatctcttcagaccatgacttttcttgccttttgacgttctttgacattttttttgaattttttgttgcaagccagatctggtgtgttatgtaataggaacaggtaaataagtctttagcttgcagacttatcttaatctgactaactattagactgtgtttaaagtctgttataaccataggtgctaaatttcttcaaattcctctagtgtctttgttttgtttgttcatgtgtttttccccttcttgagttcaggcttccctaagtgctcctcttcagagagactttctgtctttcagctctttcctctgcaattcactgttactatactggagccctgttggtgtagtactaagctgtgggaaaggagagtgctctgtaatcttacagtgaaatctcagtcttttagtgggtctgtgtctgggacattcacagagcttctccagtggtattgcttcctcatcctcaactctctttcctggctgcagcattcccaatgtatttctttgaaggcctgccccctgttgactgttattttccctctttccttaagtgggacagggagacttcaggggctgggatgaggtttgggaattgtgcttggcagagtcctttccatctttgttaccaagaaggttcatggcttatttctcaatggatgtccctctctatctgttgccagagccacgaggaaatttttcttggatcctcataatgagaaccttggagtttcctactggaaaagcccttgaatgtgtggagtgcctcaagagcacagcccccatgggtttcttgctcacaccagtccacaaacagatgccagcaattcacccaacttaccatataaaggctcatactagtttatggctccagtgctttgactccagataaatggctattggttgcgtatctctctggatgtatctgtatctccagattttggggtggcagtttgctcaggaccttggttctctaataggtctaataagaaaagtcattgattttcagctttccaactttccagctttgtcttgttataagcatggcagcaacatcttccatgccttaacatgatgacactaaaggcagaagtcgatctccatgtataaacattttaacacatatgttttttgttatcgtggtttctgacctgtctctttgccctgactttctgatactgcactagggttcctgttgctggactccattccatatgacttgctctcgtctaggctgctctttggctcatctttataaatcatgatccaaaatgaagcacatatttattttttaaataaatatgaaatgaagtatagacatcaaactgaagatgagtagatcatactgagtttcactgtctgtgcttggatcaacatcaggccttatacaaatattcaagtccagaggcaaaaggtaataaggaaaatttgtagcacaagccacaaggagataacatgtcaagtctatgcgattggaaataaactaaagatgaactgctggggatgctcactcatcacagagctcagtctaaagcaccagatttcacaagcattttttgggggaaattctgttaaaatgaaatatgagtcacatggtggtgtttcactcatcatatgtgttcaatattaattcattttaaggtttagttgcacaaaaggtaaatgagaattagaagactccatgggtaagaggagccacagaagtaaagcattgtcaagggttctatgtctatatatttagatattaggcttctgagaaaaaaacacaataggaaggaagatgaacacaacagagggcagaaggtctatacgtcctgaggccttttatgcaacgtttgtttgtggaatgttttttaagaatgtgtgagagtcattttaatgtgaaataaagacctacgtctaca

SEQ ID NO: 92 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 93 - RNA, 1968 nt, Homo sapiens
  source 1..1968: mol_type = mRNA
gaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaacggcttcttggcgtttctgcggggctggggtgttaagtggactggacttttctcgagggattcgaaggggacgggaatcttgtcaccccgggatctggcacccatggtggagtccagtgtggccttagctcccaagcctgcccctcccgagtccactctggctcaattaccccgagaaggagagagcaagtcgcggccacagcgagtgagtgaaccggagcccagatgagagcgctttaatggggctgcgaggtggcggagacagggtcgggatggggtgcagcagttggagacacagggtcagggcccctcatcctctattcactccaccggggcagtgaaagggtcccggcagcgagtgggtc

SEQ ID NO: 94 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 95 - RNA, 1140 nt, Homo sapiens
  source 1..1140: mol_type = mRNA
ggacttgggcggcccctccgcaggcggaccgggggcaaaggaggtggcatgtcggtcaggcacagcagggtcctgtgtccgcgctgagccgcgctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctagagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaagcttatttttataaagctttttcataaaa

SEQ ID NO: 96 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 97 - RNA, 1713 nt, Homo sapiens
  source 1..1713: mol_type = mRNA
gaccaggagtcagtttggcggttatgtgtggggaagaagctgggaagtcaggggctgtttctgtggacagctttccctgtcctttggaaggcacagagctctcagctgcagggaactaacagagctctgaagccgttatatgtggtcttctctcatttccagcagagcaggctcatatgaatcaaccaactgggtgaaaagataagttgcaatctgagatttaagacttgatcagataccatctggtggagggtaccaaccagcctgtctgctcattttccttcaggctgatcccataatgcatcctcaagtggtcatcttaagcctcatcctacatctggcagattctgtagctggttctgtaaaggttggtggagaggcaggtccatctgtcacactaccctgccactacagtggagctgtcacatccatgtgctggaatagaggctcatgttctctattcacatgccaaaatggcattgtctggaccaatggaacccacgtcacctatcggaaggacacacgctataagctattgggggacctttcaagaagggatgtctctttgaccatagaaaatacagctgtgtctgacagtggcgtatattgttgccgtgttgagcaccgtgggtggttcaatgacatgaaaatcaccgtatcattggagattgtgccacccaaggtcacgactactccaattgtcacaactgttccaaccgtcacgactgttcgaacgagcaccactgttccaacgacaacgactgttccaatgacgactgttccaacgacaactgttccaacaacaatgagcattccaacgacaacgactgttctgacgacaatgactgtttcaacgacaacgagcgttccaacgacaacgagcattccaacaacaacaagtgttccagtgacaacaactgtctctacctttgttcctccaatgcctttgcccaggcagaaccatgaaccagtagccacttcaccatcttcacctcagccagcagaaacccaccctacgacactgcagggagcaataaggagagaacccaccagctcaccattgtactcttacacaacagatgggaatgacaccgtgacagagtcttcagatggcctttggaataacaatcaaactcaactgttcctagaacatagtctactgacggccaataccactaaaggaatctatgctggagtctgtatttctgtcttggtgcttcttgctcttttgggtgtcatcattgccaaaaagtatttcttcaaaaaggaggttcaacaactaagtgtttcatttagcagccttcaaattaaagctttgcaaaatgcagttgaaaaggaagtccaagcagaagacaatatctacattgagaatagtctttatgccacggactaagacccagtggtgctctttgagagtttacgcccatgagtgcagaagactgaacagacatcagcacatcagacgtcttttagaccccaagacaatttttctgtttcagtttcatctggcattccaacatgtcagtgatactgggtagagtaactctctcactccaaactgtgtatagtcaacctcatcattaatgtagtcctaattttttatgctaaaactggctcaatccttctgatcattgcagttttctctcaaatatgaacactttataattgtatgttctttttagaccccataaatcctgtatacatcaaagagaa

SEQ ID NO: 98 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 99 - RNA, 2097 nt, Homo sapiens
  source 1..2097: mol_type = mRNA
gctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 100 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 101 - RNA, 696 nt, synthetic construct
  source 1..696: mol_type = other RNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 102 - protein, 232 aa, synthetic construct
MVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYK

SEQ ID NO: 103 - RNA, 3861 nt, synthetic construct
  source 1..3861: mol_type = other RNA
ggcctaactggcctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagcttggcaatccggtactgttggtaaagccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 104 - protein, 171 aa, synthetic construct
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 105 - RNA, 1634 nt, Homo sapiens
  source 1..1634: mol_type = mRNA
agtctctcgtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgtccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttgggggggggggggtcttcgacattgccgaggctggtcttgaactcctggacttagacgatcctcctgcctcagcctcccaagcaactgggattcatcctttctattaattcattgtacttatttgcttatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcctccc

SEQ ID NO: 106 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 107 - RNA, 2744 nt, Homo sapiens
  source 1..2744: mol_type = mRNA
atcacttgtgaatttttgttttccacagctctcatttctccaaaaatgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctagagacttgatttgatctcctcattcccttcagcacatgtagaggtgccagtgggtggattggagggagaagatattcaatttctagagtttgtctgtctacaaaaatcaacacaaacagaactcctctgcacgtgaattttcatctatcatgcctatctgaaagagactcaggggaagagccaaagacttttggttggatctgcagagatacttcattaatccatgataaaacaaatatggatgacagaggacatgtgcttttcaaagaatctttatctaattcttgaattcatgagtggaaaaatggagttctattcccatggaagatttacctggtatgcaaaaaggatctggggcagtagcctggctttgttctcatattcttgggctgctgtaattcattcttctcatactcccatcttctgagaccctcccaataaaaagtagactgataggatggccacagatatgcctaccataccctactttagatatggtggtgttagaagataaagaacaatctgagaactattggaatagaggtacaagtggcataaaatggaatgtacgctatctggaaatttctcttggttttatcttcctcaggatgcagggtgctttaaaaagccttatcaaaggagtcattccgaaccctcacgtagagctttgtgagaccttactgttggtgtgtgtgtctaaacattgctaattgtaaagaaagagtaaccattagtaatcattaggtttaaccccagaatggtattatcattactggattatgtcatgtaatgatttagtatttttagctagctttccacagtttgcaaagtgctttcgtaaaacagttagcaattctatgaagttaattgggcaggcatttgggggaaaattttagtgatgagaatgtgatagcatagcatagccaactttcctcaactcataggacaagtgactacaagaggcaatgggtagtcccctgcattgcactgtctcagctttagaattgttatttctgctatcgtgttataagactctaaaacttagcgaattcacttttcaggaagcatattcccctttagcccaaggtgagcagagtgaagctacaacagatctttcctttaccagcacactttttttttttttcctgcctgaatcagggagatccaggatgctgttcaggccttatcccaaccaaattcccctcttcactttgcagggcccatcttagtcaaatgtgctaacttctaaaataataaatagcactaattcaaaatttttggactcttaaattagctacttgcaggttcttgttgaaaggtatataatattacattgtaaacaaatttaaaatatttatggatatttgtgaaaagctgcattatgttaaataatattacatgtaaagctatttaaaagaggttttttttgtattttgtttaacaaaaattgctcaggagcatgctaagcctgaggccaagttgtttcttagtatgactttttaaaaaaacatctgctgagtagctacagggccaaagacttggagagcttgtttctgttgcatttgcatatcttctcaggaaattaaagtgtgtcatacatatgtgtgtgtgtgtgtgtgtgtgtgtgtatatgtgtgtgtgtatatatatgtatacttataaaatcttggtgttcttgatctttgttgtgttataagcaatgtgtgctggagtgggctggtgctagcttataagcacatattattaaattttcaggaatgttgcactttagttattaactataggcattcttgaaattggctatggtgggagtatttataccatgtaaattggcaaacactacacattttccttttggacagctagttcaccagcacaccactgtgaaactctccttaatgactcctctctgcccccgcttcattcctgggataatcatagcagactaagggagaaaatgaaattgtaaaaatttggcatactggtgatttctcagggcaagcagaggttactacagctgcagctagagggatgactaccaacaggtgacctttacattttcctgatgttataattttagcttttgttttcaatgtatactgttttcctgtttctccacatagtagtctgcattttaaatctataataaaacatgctgataactgg

SEQ ID NO: 108 - protein, 177 aa, Homo sapiens
MCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 109 - RNA, 3492 nt, Homo sapiens
  source 1..3492: mol_type = mRNA
caatcgccttttatctctggccctgggacctttgcctattttctgattgataggctttgttttgtctttacctccttctttctggggaaaacttcagttttatcgcacgttccccttttccatatcttcatcttccctctacccagattgtgaagatggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttgaggggctgatggcaatatctaaaaccaggcaccagcatgaacaccaagctgggggtggacagggcatggattcttcattgcaagtgaaggagcctcccagctcagccacgtgggatgtgacaagaagcagatcctggccctcccgcccccacccctcagggatatttaaaacttattttatataccagttaatcttatttatccttatattttctaaattgcctagccgtcacaccccaagattgccttgagcctactaggcacctttgtgagaaagaaaaaatagatgcctcttcttcaagatgcattgtttctattggtcaggcaattgtcataataaacttatgtcattgaaaacggtacctgactaccatttgctggaaatttgacatgtgtgtggcattatcaaaatgaagaggagcaaggagtgaaggagtggggttatgaatctgccaaaggtggtatgaaccaacccctggaagccaaagcggcctctccaaggttaaattgattgcagtttgcatattgcctaaatttaaactttctcatttggtgggggttcaaaagaagaatcagcttgtgaaaaatcaggacttgaagagagccgtctaagaaataccacgtgctttttttctttaccattttgctttcccagcctccaaacatagttaatagaaatttcccttcaaagaactgtctggggatgtgatgctttgaaaaatctaatcagtgacttaagagagattttcttgtatacagggagagtgagataacttattgtgaagggttagctttactgtacaggatagcagggaactggacatctcagggtaaaagtcagtacggattttaatagcctggggaggaaaacacattctttgccacagacaggcaaagcaacacatgctcatcctcctgcctatgctgagatacgcactcagctccatgtcttgtacacacagaaacattgctggtttcaagaaatgaggtgatcctattatcaaattcaatctgatgtcaaatagcactaagaagttattgtgccttatgaaaaataatgatctctgtctagaaataccatagaccatatatagtctcacattgataattgaaactagaagggtctataatcagcctatgccagggcttcaatggaatagtatccccttatgtttagttgaaatgtccccttaacttgatataatgtgttatgcttatggcgctgtggacaatctgatttttcatgtcaactttccagatgatttgtaacttctctgtgccaaaccttttataaacataaatttttgagatatgtattttaaaattgtagcacatgtttccctgacattttcaatagaggatacaacatcacagaatctttctggatgattctgtgttatcaaggaattgtactgtgctacaattatctctagaatctccagaaaggtggagggctgttcgcccttacactaaatggtctcagttggatttttttttcctgttttctatttcctcttaagtacaccttcaactatattcccatccctctattttaatctgttatgaaggaaggtaaataaaaatgctaaatagaagaaattgtaggtaaggtaagaggaatcaagttctgagtggctgccaaggcactcacagaatcataatcatggctaaatatttatggagggcctactgtggaccaggcactgggctaaatacttacatttacaagaatcattctgagacagatattcaatgatatctggcttcactactcagaagattgtgtgtgtgtttgtgtgtgtgtgtgtgtgtgtatttcactttttgttattgaccatgttctgcaaaattgcagttactcagtgagtgatatccgaaaaagtaaacgtttatgactataggtaatatttaagaaaatgcatggttcatttttaagtttggaatttttatctatatttctcacagatgtgcagtgcacatgcaggcctaagtatatgttgtgtgtgttgtttgtctttgatgtcatggtcccctctcttaggtgctcactcgctttgggtgcacctggcctgctcttcccatgttggcctctgcaaccacacagggatatttctgctatgcaccagcctcactccaccttccttccatcaaaaatatgtgtgtgtgtctcagtccctgtaagtcatgtccttcacagggagaattaacccttcgatatacatggcagagttttgtgggaaaagaattgaatgaaaagtcaggagatcagaattttaaatttgacttagccactaactagccatgtaaccttgggaaagtcatttcccatttctgggtcttgcttttctttctgttaaatgagaggaatgttaaatatctaacagtttagaatcttatgcttacagtgttatctgtgaatgcacatattaaatgtctatgttcttgttgctatgagtcaaggagtgtaaccttctcctttactatgttgaatgtatttttttctggacaagcttacatcttcctcagccatctttgtgagtccttcaagagcagttatcaattgttagttagatattttctatttagagaatgcttaagggattccaatcccgatccaaatcataatttgttcttaagtatactgggcaggtcccctattttaagtcataattttgtatttagtgctttcctggctctcagagagtattaatattgatattaataatatagttaatagtaatattgctatttacatggaaacaaataaaagatctcagaattcacta

SEQ ID NO: 110 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 111 - RNA, 911 nt, Homo sapiens
  source 1..911: mol_type = mRNA
agagaggggcaggctggtcccctgacaggttgaagcaagtagacgcccaggagccccgggagggggctgcagtttccttccttccttctcggcagcgctccgcgcccccatcgcccctcctgcgctagcggaggtgatcgccgcggcgatgccggaggagggttcgggctgctcggtgcggcgcaggccctatgggtgcgtcctgcgggctgctttggtcccattggtcgcgggcttggtgatctgcctcgtggtgtgcatccagcgcttcgcacaggctcagcagcagctgccgctcgagtcacttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctgaccactgctgctgattagggttttttaaattttattttattttatttaagttcaagagaaaaagtgtacacacaggggccacccggggttggggtgggagtgtggtggggggtagtggtggcaggacaagagaaggcattgagctttttctttcattttcctattaaaaaatacaaaaatca

SEQ ID NO: 112 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 113 - RNA, 3779 nt, Homo sapiens
  source 1..3779: mol_type = mRNA
gtcattttcctacgcgccctctgacatcagccaccttctctgtagctagtttctctgcacacaacttaatccctggcaatgaaaaatgaacctctcccccacccttgctgccgcctctcgcctcacgcccccagagaagagtttctccaccaggcagcaggtgaaggtttttttccaagtcacatgattcaggattcagggggagaatccttcttggaacagagatgggcccagaactgaatcagatgaagagagataaggtgtgatgtggggaagactatataaagaatggacccagggctgcagcaagcactcaacggaatggcccctcctggagacacagccatgcatgtgccggcgggctccgtggccagccacctggggaccacgagccgcagctatttctatttgaccacagccactctggctctgtgccttgtcttcacggtggccactattatggtgttggtcgttcagaggacggactccattcccaactcacctgacaacgtccccctcaaaggaggaaattgctcagaagacctcttatgtatcctgaaaagggctccattcaagaagtcatgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactgaacagtttctcttggccttcaggaagaaagcgcctctctaccatacagtatttcatccctccaaacacttgggcaaaaagaaaactttagaccaagacaaactacacagggtattaaatagtatacttctccttctgtctcttggaaagatacagctccagggttaaaaagagagtttttagtgaagtatctttcagatagcaggcagggaagcaatgtagtgtggtgggcagagccccacacagaatcagaagggatgaatggatgtcccagcccaacctctaattcactgtatggtcttgatctatttcttctgttttgagagcctccagttaaaatggggctccagtaccagagcagctagcaactctgccctaatgggaaatgaaggggagctgggtgtgagtgtttacactgtgcccttcacgggatacttcttttatctgcagatggcctaatacttagttgtccaagtcgcgatcaaggactctctcacacaggaaacttccctatactggcagatacacttgtgactgaaccatgcccagtttatgcctgtctgactgtcactctggcactaggaggctgatcttgtactccatatgaccccacccctaggaacccccagggaaaaccaggctgggacagccccctgttcctgagatggaaagcacaaatttaatacaccaccacaatggaaaacaagttcaaagacttttacttacagatcctggacagaaagggcataatgagtctgaagggcagtcctccttctctaggttacatgaggcaggaataagaagtcagacagagacagcaagacagttaacaatgtaggtaaagaaatagggtgtggtcactctcaattcactggcaaatgcctgaatggtctgtctgaaggaagcaacagagaagtggggaatccagtctgctaggcaggaaagatgcctctaagttcttgtctctggccagaggtgtggtatagaaccagaaacccatatcaagggtgactaagcccggcttctggtatgagaaattaaacttgtatacaaaatggttgccaaggcaacataaaattataagaattcactataccttcccctccctggaactcaggatccaagtctagaaaatgaaaggactgggtttgaattgcttcaaaacctcttccatctcagaagaccagaccctgggaactgagattccagacacaattttggaagctctccaaccaaaataaggcccccctaccccagtatataattgaagacactagtaacacctgactgcatctcatctcagcagagccagaatatggggacaaggttcagggtgccctgctgaatggtgtgaacagcaggatctcaaggatgtaatggaaagaactaccacactgaccatccagaatctaagagaccatctgggtgtttgggaaaccatctgacgaggcctgactctattccagttagattgacaataattgagcagcaggcatttttcatttctggtcaggaaagcattgtgcctttagcaaacaatcagtgtgcaacagtgatgtggtcatctagccagggaatggctgctccatcccctgcataatatattcctgcttcaaacacctctcagaaaaccagttccgcgagggtttttatatccccacaaagttgttgagagacaatgatgaccctggaagtggggaggaggacttctgagaaacagcaacctctctcctgattggggtagccatgagatttctctagctatatccaacttggcatctgtacatcatctttggaggaacatcttatttgtggaaggaccttgacaagccgtttgagatggaatgtaggccctgatgttatgcttcagtaaaaaaagatggaagcttccctgctataccaaaacatggagcaaaatttgcatttttctcaagaaggagagaaaaggagtaggactccagcaaagtttgtcagaaggaaagctagaaaagatttaaaagaaaaaaagaaagaacaaatcagcagtggtggtatggatgaaagggacttgagagaacaaaaatggctaagggaaaattttaagtcatctgctgagcagtgtgctgtgtcaacctcctcctaggtctcctctatgaaatatttagtaaagtctacatttctctttaactctttctgtgagtagattctttgggagaagcaggcattggaagaggtgttgaattcagcaagccaaatggtctgtggtaaaaaacaaaacagactttgagactcaaggctaaaaaaacagggaaatggctggcatttgagtcacacactaactgcataggacaaatgaatcttgcttaaaccaactcatgcattcttgaaaaggtatatgcaacccaactgtgtgttaactaagcaatttttttgccatctcacattctaactcgagaaagattccattttcatttttcaccaactgttctctgagcagaggtacctgacttttgcactgtgagtggtttctaatctcagtctctgtcaagcaatgctaagaaagccaacacctaaagacacaaggggtacatcatttaaatgaataatgtaaccaaacaaacaaaaaaagagaataatcattaataactcaactgatagatatgtagggagtaggcaacccaggaagtttaaaactaaattctgttactcttgagggttaaccagcccctgggaatgttatgagcaaatgatactccatgagtaaaatgatatctatgcaagtaaaataaataatttatctaactgggaa

SEQ ID NO: 114 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 115 - RNA, 1852 nt, Homo sapiens
  source 1..1852: mol_type = mRNA
aatcctgagtaaggtggccactttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatta

SEQ ID NO: 116 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 117 - RNA, 4778 nt, Homo sapiens
  source 1..4778: mol_type = mRNA
cgagactccatctcaaaaacaaaacaaataaacgaacaaaaaaacccacaacgtattattttcttgtttacgaggtttcttgtctctctggctccaccagaagaggagcagggacccttcttgctgttgttcattgctgcatcccccacaccgagagcagagcctggcatgggcagaaagtcctcagtcgatatttggtggccccaagcgaatgaagcatccaagaagggaaagctgggggctccccactgcacttgccacctgagtcacattttcagaagcctctggaaagtcgtgcacagcccaggagtgttgagcaatttcggtttcctctgaggttgaaggacccaggcgtgtcagccctgctccagacaccttgggcatggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgtgaaggaaggagcgtggtgcattggacatgggtctgacacgtggagaactcagagggtgcctcaggggaaagaaaactcacgaagcagaggctgggcgtggtggctctcgcctgtaatcccagcactttgggaggccaaggcaggcggatcacctgaggtcaggagttcgagaccagcctggctaacatggcaaaaccccatctctactaaaaatacaaaaattagccggacgtggtggtgcctgcctgtaatccagctactcaggaggctgaggcaggataattttgcttaaacccgggaggcggaggttgcagtgagccgagatcacaccactgcactccaacctgggaaacgcagtgagactgtgcctcaaaaaaaagaaaggaagaaaaaagaaaactcaggaaacagatcttgggggacactccagggaacccaaaactcaaaggcggagagctcagtgggcaccaccaaggcgagatgaagccccagcaggcaccttcagaagacccacgtagactgcagaccctgccacggacaatactaaggacaaaaacccagagacttggggtctgtgggcccccaaacatggggtaaagttgatttgcctgatattcaggaagaaggggtgaggggtgggtatttatgcttttgattcagaagaaagtggggcttgggattccagggacttggctgggggtgggaaacttcatccacttccctactctcatcatgagtacggacagggtgggcgggagactgatcatcgggactcatcatgaagagcccagccccaccccacatactcagatcccacccacagactggtggccacacctcagcctggtcacaaagagttacactcagatacatgagcacggcagcgtgctcataactgtttaacaaccagctgtcctgggagggggacagctttgtaatgtttgccaatttccatggtgtaaatgctaccaccatggctgatttcatcactgccaagcatagacatccctaataggacaccacggatctgtccccggcatccggcccagggcctggcacaaagcatgctctagggaaatgcttgctgattgaaaggaaggaagaatgactctacagtcacacctatggcatcccacaaaatctgtcacatggctgcataatctcagccactctttcacaactatagactcatacacgcgaagtgccagattcatgcacaaccacacaatcacatggaagtcacagacggcatcacagacagtcacagcactgtgtgtatgttataacacaagcacacaaaactcagacagcatcccagctacacagccactcccagaggtgtcaccgtcacacttggtaattaatactcattacattagacacagacagaccaagttatagtcagacctggttacacacatacacacacacaatatcaccatgacaaatacacattacacacacacaacatcacaatgacaaacacacattacacacacaacatcacgatgacaaacacacattacacacacaacatcacgatgacaaacacacattacacacacatcacaatgacaaacacaacattacacacacacaacatcacaatgacacacacatcacacacacatcacaatgacaaacacacaacattacacacatatacacacagcctgagggccctccccagcccagactaacacatctcggggtgaggaccagaccttgttcataaccctgggcctcttaaccactgatctttgaaataaatggcaaatagttgtacctggatctgtctagttcttaggggaacaaactgaagaagggtggagaggaattgtcaggcctaaagagccccacagggaaagggaggagtcggatggggggcaaccatcagcaacaagtggtggctcctagaggcagagggatggaggtaatgacccatggaggtcattctacagatgaggaacctggacccagttggctcaagtccatgcaggaaatgtgggggaaaccagagacctcacgtctggatctggcttcctctccaatccacaattcctgaggaagtagaggctacatcccgcaagacgcccttattagacacatccaggacagaatgacaatccgccaagccagctggaagcataaaacacagggagctggtgggttgggtgggggcagataatgatatgcatacaaattagagggtctatgcaaatgagcattgctgcagtgtggctggagggaatccttagttcctaggattctaggatatgggtttcgaccccagaggtgaatgtattgttattattgttttgttgttgttgtgaatgacaagtcaaaatttgtgggttattgttgttatcgccaatagtattcttgtcattgttgcacagtacagagatgaaggaaacagattttgcaatcagatgatcctgggttctgagtccactctgccactcaccagctatatgacctccagcaatttccatcacctctcaatgcttcagtttccccatcggcaagatggttgtggggggagaggaacaacagtacagattcaccatcccaaattcaaaatgctccaaaatctaggccgggcgtggtggctcatacctgtaatcccagcactttgggaggtcaaagtggacggataacctgaggtcaggagctccagaccagcctggccaacatggcgaaaccccatctctactaaaaatacaaaaaattacctgggtgtggtggggggcacctgtaaccccagctactcgggaggctgaggcaggaaccctggaggttgaggttgcagtgagctgagatcacaccactgcactccagcctgggtgacagagcaaggctcccatctcaaaaaacaaaaaaacatgctccaaaatctgaaactctttgagccccagtgtgatgccacaagtgggaaattccacaactcatcacatgtgatagattgcagtggaaatgcaggcacacaccacgaagtttactcagcatcctcaaaggaaatccccgtcagtagctatatatcattttctcacatgccagataggtatctctcatcttttactgttaggtacttctgtgttgaataggtggaggaaaatgattgctggttagtagtatataaattcagagtcaggaaggatggtgatgtcggctgggtgcagtggctcatgcctgtaattccaatgtgataccctaccttgtgtttaacgtgattgactctcccttagctgagagggccaggcagactctattttggcttcttcgcttgcagtctctcacccaccccccttcctcaaggacttaagctgactcccagcacatccaagaatgcgattactgataagatactgtgacaagctatatccacaattcccaggaattcgtccggttgatagcacccaaagcccccgcgtctatcaccttgtgatagatttaaagcccctgcacctggaactgtttgtttttctgttaccatttatctttttcactttcttgcctgttttgcttctgtaaaattgcttcagctcggctccctcttccccttctaaaccaaggtataaaaagaaacctagccccttctttggggtggagagaattttgagcgctagccgtctctcagtcgccggctaataaaggactcctgaattagtctaa

SEQ ID NO: 118 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 119 - RNA, 1876 nt, Homo sapiens
  source 1..1876: mol_type = mRNA
gaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaagacttctaatctttttttcataatatctttgacacatattacagaagaaataaatttcttacttttaatttaatatga

SEQ ID NO: 120 - protein, 281 aa, Homo sapiens [sequence omitted]

SEQ ID NO: 121 - RNA, 2201 nt, Homo sapiens
  source 1..2201: mol_type = mRNA
gcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccctgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacggggtgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtaccttttttgttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaacctgc

SEQ ID NO: 122 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 123 - RNA, 6583 nt, Homo sapiens
  source 1..6583: mol_type = mRNA
agaggtgcctccaggagcagcaggagcatggccgaggatctgggactgagctttggggaaacagccagtgtggaaatgctgccagagcacggcagctgcaggcccaaggccaggagcagcagcgcacgctgggctctcacctgctgcctggtgttgctccccttccttgcaggactcaccacatacctgcttgtcagccagctccgggcccagggagaggcctgtgtgcagttccaggctctaaaaggacaggagtttgcaccttcacatcagcaagtttatgcacctcttagagcagacggagataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactataggaggagagcaaatatcattatatgaaagtcctctgccaccgagttcctaattttctttgttcaaatgtaattataaccaggggttttcttggggccgggagtagggggcattccacagggacaacggtttagctatgaaatttggggcccaaaatttcacacttcatgtgccttactgatgagagtactaactggaaaaaggctgaagagagcaaatatattattaagatgggttggaggattggcgagtttctaaatattaagacactgatcactaaatgaatggatgatctactcgggtcaggattgaaagagaaatatttcaacaccttcctgctatacaatggtcaccagtggtccagttattgttcaatttgatcataaatttgcttcaattcaggagctttgaaggaagtccaaggaaagctctagaaaacagtataaactttcagaggcaaaatccttcaccaatttttccacatactttcatgccttgcctaaaaaaaatgaaaagagagttggtatgtctcatgaatgttcacacagaaggagttggttttcatgtcatctacagcatatgagaaaagctacctttcttttgattatgtacacagatatctaaataaggaagtatgagtttcacatgtatatcaaaaatacaacagttgcttgtattcagtagagttttcttgcccacctattttgtgctgggttctaccttaacccagaagacactatgaaaaacaagacagactccactcaaaatttatatgaacaccactagatacttcctgatcaaacatcagtcaacatactctaaagaataactccaagtcttggccaggcgcagtggctcacacctgtaatcccaacactttgggaggccaaggtgggtggatcatctaaggccgggagttcaagaccagcctgaccaacgtggagaaaccccatctctactaaaaatacaaaattagccgggcgtggtagcgcatggctgtaatcctggctactcaggaggccgaggcagaagaattgcttgaactggggaggcagaggttgcggtgagcccagatcgcgccattgcactccagcctgggtaacaagagcaaaactctgtccaaaaaaaaaaaaataaaataataactccaagcctttaaaaaatatcatctgaaactgttacatcagatttctggcactctactgactgtggaagatagccagctgactggaagatagccagctgattagttccctgaagaaacctgaagacagatacctggttaactagatcaactacactgccaacttgtttgatgctgagagacaatggacttattccatgggggaagggaaaaaagaagtcaatcaccaaatctgaagaagttaacctagatctttgaggtttgatttgcaactttatatgcagagtattatgtgggtattttcccttaaaatattcaaagggatttacatatgggattagctaatgagcctagccaagaccttccctggaggacaggctggtcattgcggaggtcccttctgtgcttcagtgggttcatatcctctagtccgtatgattttcctacgctaatatgtcaagggcaggagaggcagctctgttctcctagcctttgttgacttgtctgcaaagcaggaatctgcccatttgtttccaaggagcaaatgagctcatgagaatgaaagatgttaacttcatgcattctgtgccatctgagcatttcggtattatatgactggtgacccttggcccgtattataaatgcttcctatcctgggagacctcatggatgagtctgagaggaaatttggcaccaaaatcactctcactctggtttccagtagactatagaggcagagaggcatttgagaggctcctgagcaaagtgtccagtgtagcaggagcacttcattaatatttattgagttataattaaataaaaattaatttctgatttctcagtttggaggttaaggctctaaatatattttctaacctctgctaggctaacttaagccaggcctttttcttgccttccctttctcaaaacagtcagcacagactcagtgggagcacagaggagtgtggtcacctccacctggctcaccagagtcttcatagaggaagtgaagcctggaagaaactgggcgggccccagatgaccacagggaaagggcatctcagatggaggaattacccttgacttaaagcagaaaagaaagatttctcagtaactccaaaacttgcttgataggagaatattccctcaaccaattcctaggacaatatttattggtagatcaagaatgtttcctcaataactctagtctagctccatgatcagaactaacacccattaaaaacataaaatgttctttctgaaccggtcttcatggtgcgtgagagcaccaagcagctttggtatgcaggaggagttttgcacagaagagtggcctgctcaaacctgcccactgttctgtaggtgatctggtggatctggaaatttatcccaagacaggaatttcctaatattcgaagacatttgaggctttgggaaattctctgctgtgcatttatttggctcctgtcataagcttgttttttaaagaatgtatcatagctcaagtttttactgctgattttgttaaattctgtatagtatattttttacggaaaggcacagtcagacattcctaatagggctcatgtcagaacttctgttcccaaggcattatctccatagcaaaaattagtgcactgttttcaaaagtgaggtgggaaaatgcttttaagatcatgtgatgttcccctaaaaggggttaatggggtgtattcagggtttgggagggaggaagaagcatgctttagaaaacagtaaatttagggagaaaatgctttgttggttaaatgtcactcaaaaggctgaattcaaatcaattccacaaacatttactgagtacctactgcccctggggacacagagataaattatttagtctcagacacactcattctaacttcccagcacctctactgtctgcagattctttaatttattttggttgtattagctaattaattcgtaaactttaggcacatggatctattctcattatgaaaatggatgccatttgattaaggctgatgactaacaaaatgatttgtgtttactcgaagtgtttttttaaaaatagctactcaaggatagttttccataaatcaagaaggtaaaaaagttcccattttttattgtagaatccattatttaaactacatgtagagacaggttattatttgctatattcaagtttggtcatcaatacccttaaaaatattagaattttatggatgacccagaaatgctttgaaaatctgtgttcctcagcaaatacagagaccatgatcaaaatgcacagaatcactaacattttgatgctagcatggtttcagtctatttggcagaacagaattgattatgctactaaaatttctttttctttttttttttttttttttttgagacagagtcttgctttgtcacccaggctgaagtgcagtggcaggatctcagttcactgcaacctctgcctcccaggttcacgccattctcctgcttcagcctcccgagtagctgggactacaggctcccaccaccatgcccggctaattttttgcatttttagtagagacggggtttcaccgtgttagccaggatggtctcgatctcctgacctcgtgatccgcccgcctcagccttccaaagtgctgggattacaggcgtgagccactgtgcccggactctgattttttttttactaaggtacagtaagaaaagggaaaagtgtacgttttcacttcctgaaatatgtcaggttgaatcaataatagagcacaccagaactcttggctccatttcaacctaaactattcagttctcatcaccccagaggaaattccgcctctgtgctggtcagtaatccccctggattataaaagtttaactaactcactgtgcacaaggcacggccattgccaacattctcttgcaaggtattttcccaagcccttacccaattctgtttccatgattgtgacattggggattaattctgcaagacagaactgtttatattctgtaccttaaaaacacatgcaaacatctcttgccttaagatttctggctttcctatggcccagagtcctagaagtgttttgatatttgtagcagaattttcaagtgtacatccttatcctggatattaacatttttgcatcatattggcagctggacctacagagaatttagtagactgttaacctaataagccttgaatccttttgcaccagtggtgagagaatgtggatcagagccatcacctccatgccccgtcaccctctaacaaccacatttacaacttccccagctctgagacacacttgcctccaccccttccatcaccccattttaagatgaaaataccacaccagcctggaaggaagaagttacttgcccagggccacatagtgagttaagggctgatctagagctaggaagctgtcttcctgaaccataatcctggactcttctaacctctctactcatcgcaaatagagttcattttagtgatttgaaggaagatgggacaagtattttcaaacacctgtaggacaacatggaagtgggaggagacttctactgtagctccccagagaagagagctagggctacagagttgcagttacaaggttgccctctctggcttgatccccaaaggaattttctactccaaaatagaatttttctaggatgctatttctcagtccctggagatactcaaacaaagggcttgtcacaagggtttttgtagaagctattcttcacagaggttgggggagagattaagccaaaggatctctgaggtctttttcaaatctataattatgtggccttttgttcattgacttccatgtgttctagttgatcattacaaacctggcaggccttctcaagggttcagtaattagctgtcatttcccatttgtccagagagtgtccaacacaaaatacccctaagatcttggccaatagagaaatgtcatggaattttagaaatgacagtatctgcggagtttattccaagttatatcatttcaaagatgaagaaacccaggctcagagggagccatcacatccacaccctgtcacccttcgtggccagtgccagacagtagctagttggatgctaaaagtagaatttagatatcttaacaataagcccagcagtctttcaacttcattcgtaaatcatttttgttttgagcatctgtcacgtggcagcacttgcctggatactggagagctgagaaggaatgcgacaggcaagtcctactctcacagtgtatacattcaggaggaacaagacacacagtgccaagtaaataaagtagctgaacttcatcaaatgattttattcttaaagtcattaaagcatgtaatgttcccctttttttgtttcaggggtgtacagattgaagaagtgtaggtgtttatgtggttttagtgacaaaccccatgtgctttcattgattttatgttttatgttaaaacatcaaccgcaaggtaaaatgcatattgtatgttgttggatacgtacttaactggtatgcatcccatgtctttgggtactagtgtatgaattctaatctctgtaaatgaaatgttgtatgtgttaatatatttaatagatgtaacttaataaactggcattgaagactgaa

SEQ ID NO: 124 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 125 - RNA, 1805 nt, Homo sapiens
  source 1..1805: mol_type = mRNA
agcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaataa

SEQ ID NO: 126 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 127 - RNA, 2576 nt, Homo sapiens
  source 1..2576: mol_type = mRNA
aggggtagagatgcagaaaggcagaaaggagaaaattcaggataactctcctgaggggtgagccaagccctgccatgtagtgcacgcaggacatcaacaaacacagataacaggaaatgatccattccctgtggtcacttattctaaaggccccaaccttcaaagttcaagtagtgatatggatgactccacagaaagggagcagtcacgccttacttcttgccttaagaaaagagaagaaatgaaactgaaggagtgtgtttccatcctcccacggaaggaaagcccctctgtccgatcctccaaagacggaaagctgctggctgcaaccttgctgctggcactgctgtcttgctgcctcacggtggtgtctttctaccaggtggccgccctgcaaggggacctggccagcctccgggcagagctgcagggccaccacgcggagaagctgccagcaggagcaggagcccccaaggccggcctggaggaagctccagctgtcaccgcgggactgaaaatctttgaaccaccagctccaggagaaggcaactccagtcagaacagcagaaataagcgtgccgttcagggtccagaagaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtgacctacttacaccatgtctgtagctattttcctccctttctctgtacctctaagaagaaagaatctaactgaaaataccaaaaaaaaaaaaaaaaaaaaaaaaaaaaaaagtagttaccattgccttttctgtgagctatttgttttggtttgctgaaactagtccaaaacaggaaatttaacagacagccacagccaaagagtgtcatgtgaattacaagaaatagagcccatttagggaaagatagaactagaaaggcttttcattataattccatgttgaacaattgagtcatagcttcttatcttggaggaaggacacaattcaaaggggcagtaaggattttgtaaaacgtggcatccataatttactatggagcaagtgcccacatctctaggacattaagacatttatgagaaatctcaggattcatcttctgtttttatgttaaatgcactccctccttttcagttaacattataaaaagtaaaaaatgaaaattttagaaatcttgcattagacacatgaaaaaataactaaaagtttaaatttaaatatgaaacaattttgctgaaaatagtatccatatactatttaagtcttttatggttatttcaagtatacaatttctatctgtaatgtaatatattacccacacatttttttcacaggagagagagaatatcctcatttgtttatgctcatgtgtattttctatagtgaatttcagaaacttttaatatcaggtaatttcaatttatgcctataaagcattgattgaaaaataactagaattgtgcatatataacacataatctccaacagaagttactgaatacattcatactaatgtaatgtaatttccctttatttcttgctcttctgtttcaaactgctgctattgtagtttacatatcccaacctttaaaaatattcctcttattagctttatattcactttatagaagttgagttttaattaaaattcttggcatcctgaagtatgtcacatagcatgtgctccttataaatatgttgatatctcagaagacagcatcccggttttcattttataaagtaccatacttaagaatgctgtaatacttatcttttataacatgtttccttcgctttgcttgtcttttatgtcatcagttttaactgtttacttcatttaacagtttacatcattcaacagtttacttcattaaacagtaggtggaaaaatagatgccagtctatgaaaatcttcccatctatatcaaaatacttttcaaggatatacttttcaaaacaaacgatttaaattttatgtttaaaatataaactttagatttaaactttatttaaatatctggttcctatgattttgacttcagtaagttcaaataaaatatattttgcaattcatttttacattataatttaaaaagaagaagcgataagtggagtcagtttcaatgctaggtggggtggttaatgatttttctggtgttgctgctaatgtggattaacaaataaaaacattcattgccttttg

SEQ ID NO: 128 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 129 - RNA, 1811 nt, Homo sapiens
  source 1..1811: mol_type = mRNA
gtcactggcagccctgtccttcctagagggactggaacctaattctcctgaggctgagggagggtggagggtctcaaggcaacgctggccccacgacggagtgccaggagcactaacagtacccttagcttgctttcctcctccctcctttttattttcaagttcctttttatttctccttgcgtaacaaccttcttcccttctgcaccactgcccgtacccttacccgccccgccacctccttgctaccccactcttgaaaccacagctgttggcagggtccccagctcatgccagcctcatctcctttcttgctagcccccaaagggcctccaggcaacatggggggcccagtcagagagccggcactctcagttgccctctggttgagttggggggcagctctgggggccgtggcttgtgccatggctctgctgacccaacaaacagagctgcagagcctcaggagagaggtgagccggctgcaggggacaggaggcccctcccagaatggggaagggtatccctggcagagtctcccggagcagagttccgatgccctggaagcctgggagaatggggagagatcccggaaaaggagagcagtgctcacccaaaaacagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtgattgtgttataaaaagtggctcccagcttggaagaccagggtgggtacatactggagacagccaagagctgagtatataaaggagagggaatgtgcaggaacagaggcgtcttcctgggtttggctccccgttcctcacttttcccttttcattcccaccccctagactttgattttacggatatcttgcttctgttccccatggagctccgaattcttgcgtgtgtgtagatgaggggcgggggacgggcgccaggcattgtccagacctggtcggggcccactggaagcatccagaacagcaccaccatctagcggccgctcgagggaagcacccgccggttggccgaagtccacgaagccgccctctgctagggaaaacccctggttctccatgccacacctctctccaggtgccctctgcctcttcaccccacaagaagccttatcctacgtccttctctccatctatcggaccccagtttccatcactatctccagagatgtagctattatgcgcccgtctacagggggtgcccgacgatgacggtgccttcgcagtcaaattactcttcgggtcccaaggtttggctttcacgcgctccattgccccggcgtggcaggccattccaagcccttccgggctggaactggtgtcggaggagcctcgggtgtatcgtacgccctggtgttggtgttgcctcactcctctgagctcttctttctgatcaagccctgcttaaagttaaataaaatagaatgaatgata

SEQ ID NO: 130 - protein, 250 aa, Homo sapiens [sequence omitted]

SEQ ID NO: 131 - RNA, 1377 nt, Homo sapiens
  source 1..1377: mol_type = mRNA
ctctccccggcccgatccgcccgccggctccccctcccccgatccctcgggtcccgggatgggggggcggtgaggcaggcacagccccccgcccccatggccgcccgtcggagccagaggcggagggggcgccggggggagccgggcaccgccctgctggtcccgctcgcgctgggcctgggcctggcgctggcctgcctcggcctcctgctggccgtggtcagtttggggagccgggcatcgctgtccgcccaggagcctgcccaggaggagctggtggcagaggaggaccaggacccgtcggaactgaatccccagacagaagaaagccaggatcctgcgcctttcctgaaccgactagttcggcctcgcagaagtgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactgaggggccctggtctccccgcagtcgtcccaggctgccggctcccctcgacagctctctgggcacccggtcccctctgccccaccctcagccgctctttgctccagacctgcccctccctctagaggctgcctgggcctgttcacgtgttttccatcccacataaatacagtattcccactcttatcttacaactcccccaccgcccactctccacctcactagctccccaatccctgaccctttgaggcccccagtgatctcgactcccccctggccacagacccccagggcattgtgttcactgtactctgtgggcaaggatgggtccagaagaccccacttcaggcactaagaggggctggacctggcggcaggaagccaaagagactgggcctaggccaggagttcccaaatgtgaggggcgagaaacaagacaagctcctcccttgagaattccctgtggatttttaaaacagatattatttttattattattgtgacaaaatgttgataaatggatattaaatagaa

SEQ ID NO: 132 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 133 - RNA, 1678 nt, Homo sapiens
  source 1..1678: mol_type = mRNA
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 134 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 135 - RNA, 1416 nt, Homo sapiens
  source 1..1416: mol_type = mRNA
gctccgcacagcaggtgaggctctcctgccccatctccttgggctgcccgtgcttcgtgctttggactaccgcccagcagtgtcctgccctctgcctgggcctcggtccctcctgcacctgctgcctggatccccggcctgcctgggcctgggccttggttctccccatgacaccacctgaacgtctcttcctcccaagggtgtgtggcaccaccctacacctcctccttctggggctgctgctggttctgctgcctggggcccaggggctccctggtgttggcctcacaccttcagctgcccagactgcccgtcagcaccccaagatgcatcttgcccacagcaccctcaaacctgctgctcacctcattggagaccccagcaagcagaactcactgctctggagagcaaacacggaccgtgccttcctccaggatggtttctccttgagcaacaattctctcctggtccccaccagtggcatctacttcgtctactcccaggtggtcttctctgggaaagcctactctcccaaggccacctcctccccactctacctggcccatgaggtccagctcttctcctcccagtaccccttccatgtgcctctcctcagctcccagaagatggtgtatccagggctgcaggaaccctggctgcactcgatgtaccacggggctgcgttccagctcacccagggagaccagctatccacccacacagatggcatcccccacctagtcctcagccctagtactgtcttctttggagccttcgctctgtagaacttggaaaaatccagaaagaaaaaataattgatttcaagaccttctccccattctgcctccattctgaccatttcaggggtcgtcaccacctctcctttggccattccaacagctcaagtcttccctgatcaagtcaccggagctttcaaagaaggaattctaggcatcccaggggaccacacctccctgaaccatccctgatgtctgtctggctgaggatttcaagcctgcctaggaattcccagcccaaagctgttggtctgtcccaccagctaggtggggcctagatccacacacagaggaagagcaggcacatggaggagcttgggggatgactagaggcagggaggggactatttatgaaggcaaaaaaattaaattatttatttatggaggatggagagaggggaataatagaagaacatccaaggagaaacagagacaggcccaagagatgaagagtgagagggcatgcgcacaaggctgaccaagagagaaagaagtaggcatgagggatcacagggccccagaaggcagggaaaggctctgaaagccagctgccgaccagagccccacacggaggcatctgcaccctcgatgaagcccaataaacctcttttctctgaaa

SEQ ID NO: 136 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 137 - RNA, 893 nt, Homo sapiens
  source 1..893: mol_type = mRNA
agtctcaatgggggcactggggctggagggcaggggtgggaggctccaggggaggggttccctcctgctagctgtggcaggagccacttctctggtgaccttgttgctggcggtgcctatcactgtcctggctgtgctggccttagtgccccaggatcagggaggactggtaacggagacggccgaccccggggcacaggcccagcaaggactggggtttcagaagctgccagaggaggagccagaaacagatctcagccccgggctcccagctgcccacctcataggcgctccgctgaaggggcaggggctaggctgggagacgacgaaggaacaggcgtttctgacgagcgggacgcagttctcggacgccgaggggctggcgctcccgcaggacggcctctattacctctactgtctcgtcggctaccggggccgggcgccccctggcggcggggacccccagggccgctcggtcacgctgcgcagctctctgtaccgggcggggggcgcctacgggccgggcactcccgagctgctgctcgagggcgccgagacggtgactccagtgctggacccggccaggagacaagggtacgggcctctctggtacacgagcgtggggttcggcggcctggtgcagctccggaggggcgagagggtgtacgtcaacatcagtcaccccgatatggtggacttcgcgagagggaagaccttctttggggccgtgatggtggggtgagggaatatgagtgcgtggtgcgagtgcgtgaatattgggggcccggacgcccaggaccccatggcagtgggaaaaatgtaggagactgtttggaaattgattttgaacctgatgaaaataaagaatggaaagcttcagtgctgccgataaa

SEQ ID NO: 138 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 139 - RNA, 5235 nt, Homo sapiens
  source 1..5235: mol_type = mRNA
ccctttcccacccctcggagtagagctgcacatgcggctgctccctgctccgtcccgcccagccactgtcgcgcaggaacgggtccctgcagcccccagccgatggcaggacagtagccgcctgtcagaggtcgtgaacggctgaggcagacgcagcggctcccgggcctcaagagagtgggtgtctccggaggccatgggctacccggaggtggagcgcagggaactcctgcctgcagcagcgccgcgggagcgagggagccagggctgcgggtgtggcggggcccctgcccgggcgggcgaagggaacagctgcctgctcttcctgggtttctttggcctctcgctggccctccacctgctgacgttgtgctgctacctagagttgcgctcggagttgcggcgggaacgtggagccgagtcccgccttggcggctcgggcacccctggcacctctggcaccctaagcagcctcggtggcctcgaccctgacagccccatcaccagtcaccttgggcagccgtcacctaagcagcagccattggaaccgggagaagccgcactccactctgactcccaggacgggcaccagatggccctattgaatttcttcttccctgatgaaaagccatactctgaagaagaaagtaggcgtgttcgccgcaataaaagaagcaaaagcaatgaaggagcagatggcccagttaaaaacaagaaaaagggaaagaaagcaggacctcctggacccaatggccctccaggacccccaggacctccaggaccccagggacccccaggaattccagggattcctggaattccaggaacaactgttatgggaccacctggtcctccaggtcctcctggtcctcaaggaccccctggcctccagggaccttctggtgctgctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagattccccccattttgcctctgtccgtgccccttccctgggtttgggagccaggactcccagaacctctaagtgctgctgtggagtgaggtgtattggtgttgcagccgcagagaaatgccccagtgttatttattccccagtgactccagggtgacaaggcctgcttgactttccagaatgaccttgagttaacaggacagttgatggagccccagggtttacatgaagcagaaccttctttggttccatgttgactgacttatggcatgactcttcaaccccgaggtccctgttgtcagatctattgtttgttgcactaaaatgaggatccagggcagcaggccagagaaagcaaaggtgcactccagactctgggggtggacatctgaccccaagggggctgctgctcctctcttgggtagggtagtggctggggtggagtgggaagggagcattgcagcctaagaagaaggccagagagggaaaaggcaggtgcttttggcagagaccataagagaaacctgccaaggagcatccttggcagtgggaatgttctttctgctctatactgtggcctgcaggagggttggagtgctcttcccactccagctgacagccacaccgtggcagcttgctgggctttgggaagtttgctgtgctttggaacaatcacagggaatggccacaaacctgcccgcctaagaccctgaatccgtacttgggtcacatgactctcattttatttacagctgtgctccacactcagaaaattccctggggtcaccttctagttgcccccattcccagcctgactagaactcctgtcttctttctccatggagcctacctctgtctgagacaggtgcctaacctgggacctgtggtcatgtgagtctgggatattctttagcttacctgggcacaaacagaattttccatttattaagcagtacaaatgtttttcatccattcctaatcaaattctgtctggggacgaagggttggacgggatgacctccagaagtcccttcaatttctagtacctgtgactcttagccctcaccacagccttctaaattcccaaatcctagactgctcctgggcattagcaaggcagagcctttttacctggcctagaaagggcaaggggtgaggataggacagagggattttgttcaagtttgctgcaacccaagtggacgttaggccaggccttatctgaaaggccagcagctgatgctgtactaacccagtctttcttcactctggcttcaaaaagccacagcagagcattgtcaccgcaggtgctcatgctgctcccctaaagccaggctcaggagaagccagtgtctaggcactgagcagggatctgccccctagttcaggtccaaattcaccttcccctaaaccccaagcttcccaacagatcatatggtaggaccctcgagagccttacttcaaagtgcctgggctcagcctggtttctgggtgctagatccagcccaaacctgggaaggccagccttgtacagtctgctcctcttgttcctgaaatgtgtttccttttcaggagatggggaataatttccttcaggcagctgaaattcaccaagaacagcgggtacttatttctcagctgtgccttccctttctaagcaaccacactgcttggcccttcaagggtcagggtgagacgtgatgggctaggcctccgttgtctggttgctaatgacagccttgcaacccaaggtgaggtgaactccaggcatgtgtctggccctaactcctataaagtgcctcggacagtccgcagttgtagcagaaaccaacaagaaccactccttcatgtttggaaaataatttctcttgtattatctcctttgaagaaggcaaggctgataatatgacaaacatcattgtttagatgaggctcagagaggtagcactctcagagtgttttgaccagtttaagccgcagacctggagcttcagccaggtctgactccaaagctgttccattacaccacagcattgtgtggaatttgaggtctagagagaaccaataaaagtggtaattgggaactgaaatccttgagagttccggggagaaacccagagatgcctgatttcattcctcgatggtaatacccgtcctctcggctgccaggggctctgtggcaaaaagagtcagacatttctttggaaaacagcgaacagccttagagctcttgtgttcagaagaatcttcctggcacaatgttggagcagcaggcctctgggacccacagaacttgtggcctttatgttctttcacccatcctaggaaccagccaaccatcatgtgtagagcccctactgtgggcaaagtcctcctttcattaccctacagacagcttacaggagccagcctgcttcccacaactactagtgtgactccttatctctttccaccataccttagagactttgatactaccagggtctctcagggatggagggaagacctgaaagagaggactggttctgaggccagaaaggtgtgaggagagaggaggaaaagtcttcctaattgtgcccctaaagagcatcctgataccattctattctccagacatggaggggatgataaaggaaataggatctccactggacccttgattcattctgaaccctccaaaggaactctagagggcgagggatgatgagggaagcaataggtagctggggagccctattgctgctaagtcattggcaaagtgacaaagcaatttactgatgagagaatgtggaaatagatgtgcagtttggaattatgttggtgtgaatttgccagaggaccaatgcttgcatggagaatgggacgaggacatttgtgggcaagcagatgacagaggtttgaaggagaatggcatggcaggagtctctgccagttacttgggcttcaacagccaagctggcacaaaagacagctggcggaggctgctcggctactggttacctggagaagtagtatttgcctatttcccccttcatccatcctgagccaaatttcttttgctgaacaggaaagagctaggaaccctggaggtaaacaaagactttgatccatgtatgagtgtatgtgtttatgtaacttcctgtggatgcaaatagattcagagaaatttagagctaaaaaggcccttagagggaatctagcccaacctacattccaccctgttacttatgtagaaactgaggcccagagagggaagatgacctgccccaagtggtgagcaagcaccaacctccagactcagcagagtgagggggtaaagcagttcctgtcccacatggccatcttctttcttccacccacaaactccaggctggaagtacttggcccccttcaggagcctggccaggcagggagagagtagctgcagccttcatcagaactcttcctcctcccaaggcattctcccagctctagcctctggactggaaagcacaagactggcccagtgccagcaagtccttaggctactgtaatgctgcctcaggacccatccctgcctggaggctcctctaggccctgtgagcacaaagaagaaagctgatttttgtcttttaatccatttcaggactctctccaggagggctcggggtgtgtcatttctatattcctccagctgggattggggggtgggctttgttgtgagaatggcctggagcaggcccaatgctgcttttgggggtcagcatccagtgtgagatactgtgtatataaactatatataatgtatataaactgggatgtaagtttgtgtaaattaatggtttattctttgcaaataaaacgctttccccgtctgttcttgaaa

SEQ ID NO: 140 - protein, 391 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 141 - RNA, 1401 nt, Homo sapiens
  source 1..1401: mol_type = mRNA
atggagtttgggctgagctggctttttcttgtggcgattttaaaaggtgtccagtgtgaggtgcagttgttggagtctgggggagacttggtacagcctggggggtccctgagactctcctgtgcagcctctggattcacctttagcacttatgccatgagctgggtccgccaggctccagggaaggggctggaatgggtctcaggtattggtgatagtggtcatagcatatactatgcagactccgtgaagggccgcttcaccatctccagagacaattccaagaacacgctgtatctgcaaatgaatagcctgagagccgaggacacggccgtatattactgtgcgaccggctctcagtggccgggagactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 142 - protein, 466 aa, Homo sapiens [sequence omitted]

SEQ ID NO: 143 - RNA, 2079 nt, synthetic construct
  source 1..2079: mol_type = other RNA
atgtacaggatgcaactcctgtcttgcattgcactaagtcttgcacttgtcacgaactcgatatcggccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaagccagcggaagtggcggaggaggcggtcctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactctagaaagcttcctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaacactagtcctggagaaggtgcctatgtataccgctcagcattcagtgtgggattggagacttacgttactatccccaacatgcccattcgctttaccaagatcttctacaatcagcaaaaccactatgatggctccactggtaaattccactgcaacattcctgggctgtactactttgcctaccacatcacagtctatatgaaggatgtgaaggtcagcctcttcaagaaggacaaggctatgctcttcacctatgatcagtaccaggaaaataatgtggaccaggcctccggctctgtgctcctgcatctggaggtgggcgaccaagtctggctccaggtgtatggggaaggagagcgtaatggactctatgctgataatgacaatgactccaccttcacaggctttcttctctaccatgacaccaactaa

SEQ ID NO: 144 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - RNA, 660 nt, synthetic construct
  source 1..660: mol_type = other RNA
ccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - DNA, 7653 bp, Homo sapiens
  source 1..7653: mol_type = other DNA
tgtacatagtagttgtctagcactgtctcatagtagtagtcataggtcgtcagcgtcgtctcagtcactgtctcatcagtcgtcagcgtctcatcataggtcacttcatagtagtagagcgtcgtctcatagtagtaggtcgtcgtcgtcagctcaactgtcgtcgtcactgtctaggtcgtcagcgtctcatagactactgtcgtctaggtcactagcagcagcagcagcagcagcagcatttctagcgtcgtctcaactgtcgtctaggtcgtctcaagcgtctcatcaactgtctcatcaactgtcgtcagctaggtctcaacttcaacttcatcatcaactagcacttaggtctcatcatcagtctaggtcactagcgtctcatcatcaactgtcacttcaacttcaagctaggtcgtcacttcatcatcataggtctcaagcagcagcagcagcagcagctctgcgtctagctcataggtcgtcacttcatcatcatagtcaagcgtctaggtcgtcacttagactgtcacttcaagcactactgtcactacttcatcagtcactgtcagcgtcgtcgtctcatcatagtcataggtctcaagcgtctcatcatcataggtcgtcgtctcatcaagctcaactgtctcatagtcataggtctagtcaagcagcagcagcagcagcagctctaggtctagctcatcaacttcaacttcatcagtctcagtcagcgtctagtcaacttcaacttaggtcgtctcaagcacttcatcaacttcatcatagtcatagtcaagctagtaggtctcaactgtctcatcatagtcaagctcaacttcatcaactactgtcgtcgtctcaagctcatcaacttagtcagtcgtctagtcatagagcagcagcagcagcagcagcgcgagatctagctagtcatcatcatcatcataggtcgtctcaagcgtctcatcatcataggtctcatagtcatcaagcactgtcgtcactgtctcaacttcatcatagagctcatcagtcactgtcactgtctcaacttcaagcactgtctcagtcgtctcatcatcataggtcagcgtcgtctcataggtctcatcataggtcgtcagcagcagcagcagcagcagcgtgatatctagctagtcaactactgtcgtcacttcatagactagctcatagtagtcatcatcatcagtcactactagctcatcagtcgtctaggtcacttcagtcgtcagctaggtctagtcagtctaggtcgtcactactagctcatagtcaactgtcgtctcagtctcatagagctcataggtcacttcatcaactgtctcagtcagcagcagcagcagcagcagcgtgatttctagcgtctcagtctaggtctcaacttcaacttcaagctcatagtagtcatcatcagtcgtctcatagagcgtctagtcatcatagacttcaactgtctagagctcatcatagtcaactgtcgtcacttcatagagctcatagacttcatagtcatcatcatagtcaagcactgtctcaactgtctcagtctaggtcgtcagcagcagcagcagcagcagcagagcgtctagctaggtcacttcatcagtctaggtcacttcaagctcatagtcatcaactgtctcaactacttcaagctagtagtcagtcgtctcaacttcatcatcaagcactgtcacttcatcatagacttcaacttcaagctcataggtctcaactacttcagtctagactagcgtcacttagtcaacttcaactactgtctcaagcagcagcagcagcagcagcagaaggtctagctcatcaactgtctcaactacttcaacttcaagctcaactactgtcgtctaggtcgtcacttcaagcactactgtcacttcaactgtctagtaggtcagcgtctaggtcactgtcactgtcgtctcatcaagcactgtctcatagtcaactgtcgtcgtcactagcgtcgtcgtcactgtcgtcgtctaggtctagagcagcagcagcagcagcagccgcagatctagctcataggtctcataggtcgtcactactgtcagctcatcaactgtcgtctcatagtcaactgtcagctcatcatcatagtcatcataggtctcatcaagctaggtcgtcacttcagtctcaacttcatcaagctcatcagtcgtctcataggtctaggtctcaagcactgtctcatcatcatcaactgtctcatcaagcagcagcagcagcagcagcattatatctagctcaactgtcgtcgtctcaactgtctcaactagcactgtcgtctcaactgtcgtctcatcatcaagctcaacttagtcataggtctagtcatagtcaagctcatagtcaacttcatcatcagtcgtcactagcgtcgtctcatcatagtcataggtctcatcaagctcagtctcatcatcatcaacttcatagtcaagcagcagcagcagcagcagcattatttctagcacttaggtctcatagtcaactgtcgtcgtcagcactgtcactgtcgtcgtctagtcatagtagagctcataggtcgtctcatagtagtagtagtagagctcatcaacttcatcaactgtcgtctcatagagctcatcaactgtcgtctcaactgtcgtctcaagcacttcaactgtcgtctcataggtcgtcgtcagcagcagcagcagcagcagcaccgcgtctagctaggtctcatcatcatcatagacttcatcaagctcatcaactgtcgtctcatcatcatagtagagctcaacttcaacttcaacttcaactgtcgtcagcgtcgtctcaactgtcgtctaggtctcatagagctaggtcgtctcatagtcaactgtcacttcaagctcataggtctcatcaactactactactgtcagcagcagcagcagcagcagcaccaggtctagctcatcaacttagacttagtcatcagtcgtcagcgtcactgtcgtcacttcatcatcataggtcagctcatcatcatcataggtcacttcatcatagagcactactgtctcatcagtcacttcatcatcaagctcaactactactgtcgtctcatcaactactagcacttcataggtctagtcatcaacttcatagagcagcagcagcagcagcagcaggagatctagctcatcatcatagtcaactgtctcatagtcaagcgtcgtcacttcaacttcatcatagtagtcaagctagtcatagtcatcatagtcatcatcaactagcgtcacttagtcatcagtcactgtctagactagcacttcatagtcatcatcaactacttagtcaagctagtagtcatagtcatagtcataggtctcaagcagcagcagcagcagcagccaaatatctagcactgtcactgtctcagtctcaactactactagctaggtctagtaggtctaggtctagtcagtcagcactgtctaggtctcatcatcaacttcatcaagcgtctaggtctcatcatcaactgtcgtctagagcactactgtctcatcaactgtctcatcatcaagcactgtcgtctcatcatagtcagtctcatcaagcagcagcagcagcagcagccaaatttctagctcatagtcatcaactgtctcatagtcaactagcactgtcgtctcagtcgtcgtcacttcaactagcgtcgtctaggtctcatcatcatagactgtcagcactgtctagactgtctcatcataggtctcaagcacttagtcatcaactgtcgtcgtcacttcaagcactgtcgtctcatcatcatcaactgtctcaagcagcagcagcagcagctctatagcgtctagctaggtcgtcgtctaggtctcataggtcactagctcaacttcagtctagtcatcaacttcatcaagctagtcatcaacttagtcatagtcatagtagagctcatcatagtcaactgtctcaacttcatcaagcacttcatcataggtctaggtcgtctcaactagcgtcgtcacttcatcagtctagtcaactgtcagcagcagcagcagcagctctataaggtctagctagtcatagtagtcatcatagtcatagtagagctcatcatcatcatcatcaactactactactagctcatcatcaactactgtcgtcacttcaactagctcatcatcatagtcaacttaggtcacttagagctcatagtcatcatcagtcgtcacttcatcaagctcatcataggtcactgtcgtctagtcaactagcagcagcagcagcagctcttctagatctagctcagtctaggtctcagtctaggtcgtctagagcgtcgtctaggtcgtcacttcagtctaggtcagcactgtctcatcaacttcagtcactactgtcagcacttcatcatcatcagtcactgtcgtctagagctcatcaactgtctagtagtcaactacttcaagctaggtcgtctagacttcagtctaggtcgtcagcagcagcagcagcagctctgcgatatctagcacttcagtcgtctcaacttaggtcgtcactagcgtcgtctaggtctcaacttagactacttagagcgtctcatcaactactgtcacttcaactactagcactgtctcatcaacttcagtcgtcgtcactagcgtcgtcactgtctcaactgtctagtagtcaagcactacttcaactgtctcaacttcagtctagagcagcagcagcagcagctctgcgatttctagctagtcatcagtctaggtctaggtcgtctagagctcaactgtctcagtctagtcatcatagtcaagcacttcatcagtctagtcagtctaggtctcaagcacttcatcaactgtcgtcacttcataggtcagcgtctcataggtcactacttcagtcgtctcaagcactactgtcgtcactgtctagacttcaactagcagcagcagcagcagctctgtggcgtctagcactgtctaggtctcaactactgtcgtctagagctcatagtcatcaactacttcaactactactagcgtcgtctcatcatagtcatcatcaactgtcagctcatcatcatcatcaacttagtcagtcactagcgtcactactactacttcatcaacttagtcaagctagtcatcaactactactacttcatcaactagcagcagcagcagcagctctgtgaggtctagcactactgtcgtctaggtcgtcgtcacttcaagctcatcagtctcagtcgtcgtcgtctagactagctaggtcactgtcgtcgtctcatcaacttcaagcacttaggtcgtcacttcaactgtcactgtcagcgtctcatcagtcgtctcatagtcagtcgtcagctcatcatcaacttcatcatcatagtcatagagcagcagcagcagcagctctagaagatctagcgtctcatcatcataggtcgtcgtcactgtcagctaggtcacttcatcagtctcataggtctagagcgtctcatcaactacttcatcatagtcatagagcgtctagtcatcatcatagacttcaactgtcagcgtcgtctcaactgtctcatcatcatcagtcagcactgtcactacttcatcaacttcaactgtcagcagcagcagcagcagctctcgcatatctagcgtctaggtctagacttcaacttcatcatcaagctaggtctcatcatcatcatcaacttagtcaagctcatcagtcgtcgtcactgtcgtcactgtcagcacttaggtcacttcatcaactactgtcactagcacttcatcaactgtcgtctagtcaactgtcagctcatcataggtcacttcatcataggtctcaagcagcagcagcagcagctctcgcatttctagctcataggtcgtctagtcaactactactgtcagcgtctcatagtagtcatagacttcatcatcaagctcaactgtctcagtcacttcaacttagtcaagcgtctcatcagtctaggtcgtcactgtctagagcgtcgtcgtcactgtcactgtctcaactactagctaggtcgtcgtctcaactgtctcatcagtcagcagcagcagcagcagctctattgcgtctagcgtcactgtcactacttcaactacttcatagagcacttcaactactgtcacttcatcaacttcaagcacttcatcatagtcatcatcaacttaggtcagctcataggtcgtcacttcatagtcatcagtcagcacttcagtcgtctcatagtcatcatagtagagctcatagtagtcatcatagtcatagacttcaagcagcagcagcagcagctctattaggtctagcactgtctcaactactgtctcatagtcaactagctcatcagtctaggtcgtcacttcaactactagcgtcactgtctcaactgtcgtctaggtcgtcagctcaactgtctcaactgtcgtcgtcgtcactagcacttcagtctagtcatagtagtcatagtcaagcacttaggtctcatagtcatcagtctaggtcagcagcagcagcagcagctctaccagatctagcacttaggtctcaacttaggtcactgtcgtcagctcatagtcataggtctcaacttcaactactagctcatcaacttcatagacttcaacttcaactagctcaactgtcactactgtcactgtctcatcaagctagtcatagtcatcatcataggtctagtcaagctagtcatcagtcgtcgtctagactactactagcagcagcagcagcagctctaggatatctagctaggtcactgtctaggtctcatcaacttcaagcgtcgtctcatcaactgtctcaactactgtcagctcatcatcatcatcagtctcatagtcatcaagctcatcaactgtcgtctcatagtcatagtcaagcgtcgtcgtcgtctagtcagtctcagtctcaagcgtcactgtcgtcacttaggtctcatagtagagcagcagcagcagcagctctaggatttctagcgtcgtctcaacttcagtctagacttcatcaagctcatcagtctaggtctagacttcaacttagagcacttcatagtagtcatcatcagtcgtcgtcagctcaacttcatcatcaactgtctcaacttagagcgtcgtcactactacttagactactactgtcagctcaacttcatcatcaactgtctcagtctcaagcagcagcagcagcagctctcaagcgtctagctagtagtcatcatcataggtcgtcgtctcaagctcatcatcataggtctcagtcactgtcactagctcataggtctaggtcacttaggtcgtctagagctagtcatagtagtagtcatcaacttcagtcagcgtcgtctagtcaactgtcgtctcatcagtcagcactgtctagtcataggtcactgtcgtctcaagcagcagcagcagcagctctcaaaggtctagctcataggtcactgtctaggtcgtctcaactagctaggtcactgtcgtcgtcactgtcgtctcaagcactgtcactgtctaggtcgtcgtctagtcataatcagtagag

SEQ ID NO: 148 - protein, 282 aa, Homo sapiens
ALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 149 - RNA, 1425 nt, Homo sapiens
  source 1..1425: mol_type = mRNA
atggagtttgggctgagctgggttctccttgttgtttttttacaaggtgtccagtgtgaggtgcaactggtggactctgggggaggcttggtccagcctggagggtccctgagactctcgtgtgcagcctctggattcatcgtcagtgaccactacgtagagtgggtccgccaggctccagggaaggggccggagtgggttggttgtttcagaagcaaagctcacaagtccaccacagaatatgccgcgtctgtgaaaggcagattcaccatcttaagagatgattcgaagaactcagtgcatctccaaatgaacagcctcaaaaccgacgacacggccgtgtattattgtgttagagatcttgagggggctggtaaatacgactggtatttcgatatttggggccgaggcatcctggtcactgtctcctcagcttccaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttgagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatga

SEQ ID NO: 150 - protein, 474 aa, Homo sapiens [sequence omitted]

SEQ ID NO: 151 - DNA, 687 bp, synthetic construct
  source 1..687: mol_type = other DNA
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagggtcctcaccgtcctgcaccaggactggctgaacggtaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggaggacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctccgggtaaa

SEQ ID NO: 152 - protein, 502 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERESKYGPPCPSCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGKFYTGVYILIGAGALMMLVGFLGCCGAVQESQCVIM

SEQ ID NO: 153 - DNA, 12 bp, synthetic construct
  source 1..12: mol_type = other DNA
ggctcgagcggc

SEQ ID NO: 154 - protein, 4 aa, synthetic construct
GSSG

SEQ ID NO: 155 - DNA, 15 bp, synthetic construct
  source 1..15: mol_type = other DNA
ggtggcggtggatcc

SEQ ID NO: 156 - protein, 5 aa, synthetic construct
GGGGS

SEQ ID NO: 157 - DNA, 15 bp, synthetic construct
  source 1..15: mol_type = other DNA
ggtggcggtggctcc

SEQ ID NO: 158 - DNA, 15 bp, synthetic construct
  source 1..15: mol_type = other DNA
ggaggtggcggctcc

SEQ ID NO: 159 - DNA, 30 bp, synthetic construct
  source 1..30: mol_type = other DNA
ggtggcggtggctccggtggcggtggatcc

SEQ ID NO: 160 - protein, 10 aa, synthetic construct
GGGGSGGGGS

SEQ ID NO: 161 - DNA, 30 bp, synthetic construct
  source 1..30: mol_type = other DNA
ggtggcggtggctccggtggcggtggctcc

SEQ ID NO: 162 - DNA, 30 bp, synthetic construct
  source 1..30: mol_type = other DNA
ggtggcggtggctccggaggtggcggctcc

SEQ ID NO: 163 - DNA, 30 bp, synthetic construct
  source 1..30: mol_type = other DNA
ggtggcggtggctccggaggtggcggttcc

SEQ ID NO: 164 - DNA, 45 bp, synthetic construct
  source 1..45: mol_type = other DNA
ggtggcggtggctccggtggcggtggctccggtggcggtggatcc

SEQ ID NO: 165 - protein, 15 aa, synthetic construct
GGGGSGGGGSGGGGS

SEQ ID NO: 166 - DNA, 45 bp, synthetic construct
  source 1..45: mol_type = other DNA
ggaggtggcggttccggtggcggtggctccggtggcggtggctcc

SEQ ID NO: 167 - DNA, 45 bp, synthetic construct
  source 1..45: mol_type = other DNA
ggtggcggtggctccggtggcggtggctccggaggtggcggctcc

SEQ ID NO: 168 - DNA, 60 bp, synthetic construct
  source 1..60: mol_type = other DNA
ggtggcggtggctccggtggcggtggctccggtggcggtggctccggtggcggtggatcc

SEQ ID NO: 169 - protein, 20 aa, synthetic construct
GGGGSGGGGSGGGGSGGGGS

SEQ ID NO: 170 - DNA, 60 bp, synthetic construct
  source 1..60: mol_type = other DNA
ggtggcggtggctccggaggtggcggttccggtggcggtggctccggtggcggtggctcc

SEQ ID NO: 171 - DNA, 60 bp, synthetic construct
  source 1..60: mol_type = other DNA
ggtggcggtggctccggtggcggtggctccggtggcggtggctccggaggtggcggctcc

SEQ ID NO: 172 - DNA, 27 bp, synthetic construct
  source 1..27: mol_type = other DNA
gctactaacttcagcctgctgaagcag

SEQ ID NO: 173 - protein, 17 aa, synthetic construct
ATNFSLKQAGDVENPGP

SEQ ID NO: 174 - RNA, 2832 nt, synthetic construct
  source 1..2832: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggaggtggcggttccggtggcggtggctccggtggcggtggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggtggcggtggctccggtggcggtggctccggaggtggcggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 175 - protein, 943 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 176 - RNA, 2214 nt, synthetic construct
  source 1..2214: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctccggtggcggtggctccggaggtggcggttccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctccggtggcggtggctccggtggcggtggctccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 177 - protein, 737 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 178 - RNA, 2286 nt, synthetic construct
  source 1..2286: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtccttggtggcggtggctccggaggtggcggttcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtccttggtggcggtggctccggtggcggtggctcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 179 - protein, 761 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 180 - RNA, 2385 nt, synthetic construct
  source 1..2385: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgccccggtggcggtggctccggaggtggcggttcccttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgccccggtggcggtggctccggtggcggtggctcccttgggtgggacgtagctgagctgcagctgaatcacacaggacctcagcaggaccccaggctatactggcaggggggcccagcactgggccgctccttcctgcatggaccagagctggacaaggggcagctacgtatccatcgtgatggcatctacatggtacacatccaggtgacgctggccatctgctcctccacgacggcctccaggcaccaccccaccaccctggccgtgggaatctgctctcccgcctcccgtagcatcagcctgctgcgtctcagcttccaccaaggttgtaccattgcctcccagcgcctgacgcccctggcccgaggggacacactctgcaccaacctcactgggacacttttgccttcccgaaacactgatgagaccttctttggagtgcagtgggtgcgcccctga

SEQ ID NO: 181 - protein, 794 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGGGGSGGGGSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGGGGSGGGGSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 182 - RNA, 2340 nt, synthetic construct
  source 1..2340: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcctgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagacggtggcggtggctccggaggtggcggttcctgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagacggtggcggtggctccggtggcggtggctcctgggcctacctccaagtggcaaagcatctaaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagactga

SEQ ID NO: 183 - protein, 779 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSDGGGGSGGGGSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSDGGGGSGGGGSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 184 - RNA, 2421 nt, synthetic construct
  source 1..2421: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggtggcggtggctccggaggtggcggttccgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcggtggcggtggctccggtggcggtggctccgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctga

SEQ ID NO: 185 - protein, 806 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLGGGGSGGGGSDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 186 - RNA, 2427 nt, synthetic construct
  source 1..2427: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgggtggcggtggctccgtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgggaggtggcggctccgtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtgtga

SEQ ID NO: 187 - protein, 808 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGGGSVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMVGGGGSVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 188 - RNA, 2520 nt, synthetic construct
  source 1..2520: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggcggtcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggcggccagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 189 - protein, 839 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 190 - RNA, 2529 nt, synthetic construct
  source 1..2529: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagatggtggcggtggctccggaggtggcggttccgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagatggtggcggtggctccggtggcggtggctccgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattga

SEQ ID NO: 191 - protein, 842 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDIDGGGGSGGGGSEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDIDGGGGSGGGGSEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 192 - RNA, 2547 nt, synthetic construct
  source 1..2547: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactaggtggcggtggctccggaggtggcggttccgataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactaggtggcggtggctccggtggcggtggctccgataagccaagggcacacctgacagttgtgagacaaactcccacacagcactttaaaaatcagttcccagctctgcactgggaacatgaactaggcctggccttcaccaagaaccgaatgaactataccaacaaattcctgctgatcccagagtcgggagactacttcatttactcccaggtcacattccgtgggatgacctctgagtgcagtgaaatcagacaagcaggccgaccaaacaagccagactccatcactgtggtcatcaccaaggtaacagacagctaccctgagccaacccagctcctcatggggaccaagtctgtatgcgaagtaggtagcaactggttccagcccatctacctcggagccatgttctccttgcaagaaggggacaagctaatggtgaacgtcagtgacatctctttggtggattacacaaaagaagataaaaccttctttggagccttcttactatag

SEQ ID NO: 193 - protein, 848 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLLGGGGSGGGGSDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLLGGGGSGGGGSDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 194 - RNA, 2358 nt, synthetic construct
  source 1..2358: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctcggtggcggtggctccggaggtggcggttccaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctcggtggcggtggctccggtggcggtggctccaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 195 - protein, 785 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGGGSGGGGSRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKLGGGGSGGGGSRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 196 - RNA, 2430 nt, synthetic construct
  source 1..2430: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgggtggcggtggctccggaggtggcggttccgaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgggtggcggtggctccggtggcggtggctccgaaacagtcactcaagactgcttgcaactgattgcagacagtgaaacaccaactatacaaaaaggatcttacacatttgttccatggcttctcagctttaaaaggggaagtgccctagaagaaaaagagaataaaatattggtcaaagaaactggttacttttttatatatggtcaggttttatatactgataagacctacgccatgggacatctaattcagaggaagaaggtccatgtctttggggatgaattgagtctggtgactttgtttcgatgtattcaaaatatgcctgaaacactacccaataattcctgctattcagctggcattgcaaaactggaagaaggagatgaactccaacttgcaataccaagagaaaatgcacaaatatcactggatggagatgtcacattttttggtgcattgaaactgctgtga

SEQ ID NO: 197 - protein, 809 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLLGGGGSGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLLGGGGSGGGGSETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 198 - RNA, 2376 nt, synthetic construct
  source 1..2376: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgggtggcggtggctccggaggtggcggttcccagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgggtggcggtggctccggtggcggtggctcccagaagaagcagcactctgtcctgcacctggttcccattaacgccacctccaaggatgactccgatgtgacagaggtgatgtggcaaccagctcttaggcgtgggagaggcctacaggcccaaggatatggtgtccgaatccaggatgctggagtttatctgctgtatagccaggtcctgtttcaagacgtgactttcaccatgggtcaggtggtgtctcgagaaggccaaggaaggcaggagactctattccgatgtataagaagtatgccctcccacccggaccgggcctacaacagctgctatagcgcaggtgtcttccatttacaccaaggggatattctgagtgtcataattccccgggcaagggcgaaacttaacctctctccacatggaaccttcctggggtttgtgaaactgtga

SEQ ID NO: 199 - protein, 791 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSGGGGSQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSGGGGSQKKQHSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 200 - RNA, 2511 nt, synthetic construct
  source 1..2511: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcacggtggcggtggctccggaggtggcggttccgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcacggtggcggtggctccggtggcggtggctccgcacctaaaggccggaaaacacgggctcgaagagcgatcgcagcccattatgaagttcatccacgacctggacaggacggagcgcaggcaggtgtggacgggacagtgagtggctgggaggaagccagaatcaacagctccagccctctgcgctacaaccgccagatcggggagtttatagtcacccgggctgggctctactacctgtactgtcaggtgcactttgatgaggggaaggctgtctacctgaagctggacttgctggtggatggtgtgctggccctgcgctgcctggaggaattctcagccactgcggcgagttccctcgggccccagctccgcctctgccaggtgtctgggctgttggccctgcggccagggtcctccctgcggatccgcaccctcccctgggcccatctcaaggctgcccccttcctcacctacttcggactcttccaggttcactga

SEQ ID NO: 201 - protein, 836 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVHGGGGSGGGGSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVHGGGGSGGGGSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 202 - RNA, 963 nt, synthetic construct
  source 1..963: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtggcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 203 - protein, 320 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 204 - RNA, 2712 nt, synthetic construct
  source 1..2712: mol_type = other RNA
atgggttgctgtttctccaagaccggctcgagcggcacgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgatgtgggttatgcttcagactctcaacgatgaggttccaaaataccgtgaccagattcctagcccaggactcatggtttttccaaaaccagtgaccgcattggaatatacattcagtaggtctgatccaacttcgtatgcagggtacattgaagaccttaagaagtttctaaaaccatatactttagaagaacagaagaacctcacagtctgtcctgatggagcactttttgaacagaagggtccagtttatgttgcatgtcagtttcctatttcattacttcaagcatgcagtggtatgaatgatcctgattttggctattctcaaggaaacccttgtattcttgtgaaaatgaacagaataattggattaaagcctgaaggagtgccaaggatagattgtgtttcaaagaatgaagatataccaaatgtagcagtttatcctcataatggaatgatagacttaaaatatttcccatattatgggaaaaaactgcatgttgggtatctacagccattggttgctgttcaggtcagctttgctcctaacaacactgggaaagaagtaacagttgagtgcaagattgatggatcagccaacctaaaaagtcaggatgatcgtgacaagtttttgggacgagttatgttcaaaatcacagcacgtgcagcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggatccggtggcggtggatccggtggcggtggatccggtggcggtggatcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggatccggtggcggtggatccggtggcggtggatccggtggcggtggatcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 205 - protein, 903 aa, synthetic construct
MGCCFSKTGSSGTKNEKKSLNQSLAEWKLFIYNPTTGEFLGRTAKSWGLILLFYLVFYGFLAALFSFTMWVMLQTLNDEVPKYRDQIPSPGLMVFPKPVTALEYTFSRSDPTSYAGYIEDLKKFLKPYTLEEQKNLTVCPDGALFEQKGPVYVACQFPISLLQACSGMNDPDFGYSQGNPCILVKMNRIIGLKPEGVPRIDCVSKNEDIPNVAVYPHNGMIDLKYFPYYGKKLHVGYLQPLVAVQVSFAPNNTGKEVTVECKIDGSANLKSQDDRDKFLGRVMFKITARAACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 206 - RNA, 2172 nt, synthetic construct
  source 1..2172: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaggtggcggtggctccggtggcggtggctccggtggcggtggatcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggaggtggcggttccggtggcggtggctccggtggcggtggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggtggcggtggctccggtggcggtggctccggaggtggcggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 207 - protein, 723 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 208 - RNA, 2694 nt, synthetic construct
  source 1..2694: mol_type = other RNA
atgtggtggcgactctggtggctgttgcttctgctcttgcttctgtggcccatggtctgggcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggaggtggcggttccggtggcggtggctccggtggcggtggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggtggcggtggctccggtggcggtggctccggaggtggcggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggtggcggtggctccggtggcggtggctccggtggcggtggatccgacaaaactcacacatgcccaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaatcgatttctacacaggagtctatattctgatcggagccggcgccctcatgatgctggtgggcttcctgggctgctgcggggctgtgcaggagtcccagtgcgtaattatgtaa

SEQ ID NO: 209 - protein, 897 aa, synthetic construct
MWWRLWWLLLLLLLLWPMVWAREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKIDFYTGVYILIGAGALMMLVGFLGCCGAVQESQCVIM

SEQ ID NO: 210 - RNA, 2688 nt, synthetic construct
  source 1..2688: mol_type = other RNA
atgggttgctgtttctccaagaccggctcgagcggcacgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggaggtggcggttccggtggcggtggctccggtggcggtggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaaggtggcggtggctccggtggcggtggctccggtggcggtggctccggaggtggcggctcccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 211 - protein, 895 aa, synthetic construct
MGCCFSKTGSSGTKNEKKSLNQSLAEWKLFIYNPTTGEFLGRTAKSWGLILLFYLVFYGFLAALFSFTPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 212 - RNA, 2070 nt, synthetic construct
  source 1..2070: mol_type = other RNA
atgggttgctgtttctccaagaccggctcgagcggcacgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctccggtggcggtggctccggaggtggcggttccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctccggtggcggtggctccggtggcggtggctccgagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctag

SEQ ID NO: 213 - protein, 689 aa, synthetic construct
MGCCFSKTGSSGTKNEKKSLNQSLAEWKLFIYNPTTGEFLGRTAKSWGLILLFYLVFYGFLAALFSFTPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGGGGSGGGGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 214 - RNA, 2142 nt, synthetic construct
  source 1..2142: mol_type = other RNA
atgggttgctgtttctccaagaccggctcgagcggcacgaagaacgagaagaagtccctcaaccagagcctggccgagtggaagctcttcatctacaacccgaccaccggagaattcctggggcgcaccgccaagagctggggtttgatcttgctcttctacctagttttttatgggttcctggctgcactcttctcattcacgccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtccttggtggcggtggctccggaggtggcggttcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtccttggtggcggtggctccggtggcggtggctcccatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 215 - protein, 713 aa, synthetic construct
MGCCFSKTGSSGTKNEKKSLNQSLAEWKLFIYNPTTGEFLGRTAKSWGLILLFYLVFYGFLAALFSFTPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLGGGGSGGGGSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 216 - RNA, 2562 nt, Synthetic construct
  source 1..2562: mol_type = other RNA
atgggttgctgtttctccaagaccggctcgagcggcagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgaccgtgcccagcacctgaagcagccgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggtggcggtggctccggtggcggtggctccggtggcggtggatccttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgggtggcggtggctccggtggcggtggctccttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgggtggcggtggctccggaggtggcggctccttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataa

SEQ ID NO: 217 - protein, 853 aa, synthetic construct
MGCCFSKTGSSGSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGGGGSGGGGSLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 218 - protein, 4 aa, Synthetic construct
GGGS

SEQ ID NO: 219 - protein, 4 aa, Synthetic construct
GGGG